(19) United States
(12) Patent Application Publication
(10) Pub. No.: US 2023/0227508 A1
(43) Pub. Date: Jul. 20, 2023

(54) SPLIT INTEIN-BASED SELECTION FOR PEPTIDE BINDERS

(71) Applicant: Massachusetts Institute of Technology, Cambridge, MA (US)

(72) Inventors: Christopher A. Voigt, Belmont, MA (US); Andrew King, Cambridge, MA (US); Emerson Glassey, Malden, MA (US)

(73) Assignee: Massachusetts Institute of Technology, Cambridge, MA (US)

(21) Appl. No.: 18/009,357

(22) PCT Filed: Jun. 11, 2021
(86) PCT No.: PCT/US2021/037120

§ 371 Date: Dec. 9, 2022

Related U.S. Application Data
(60) Provisional application No. 63/038,394, filed on Jun. 12, 2020.

(51) Int. Cl. C07K 14/32; C12N 15/10

(52) U.S. Cl. CPC C07K 14/32; C12N 15/1086; C12N 15/1055; C07K 2319/92

(57) ABSTRACT

Disclosed herein, in some embodiments, non-naturally occurring proteins (e.g., non-naturally occurring modified proteins) that may be useful in the treatment of bacterial and viral infections, including SARS-CoV-2 infection, host cells comprising the same, and methods of treating bacterial and viral infections including SARS-CoV-2 infection. Also provided herein are host cells comprising fusion proteins for split intein-based selection of peptides that bind a target protein, methods of using the same, and methods of identifying peptides that bind a target protein.

### RELATED APPLICATION

This application claims the benefit under 35 U.S.C. § 119(e) of U.S. provisional application No. 63/038,394, filed Jun. 12, 2020, which is incorporated by reference herein in its entirety.

### FEDERALLY SPONSORED RESEARCH

This invention was made with Government support under Grant No. HR0011-15-C-0084 awarded by the Defense Advanced Research Projects Agency (DARPA). The Government has certain rights in this invention.

### BACKGROUND OF THE INVENTION

Protein-protein interactions play an important role in elucidating the mechanisms of biological systems and in numerous clinical applications. For example, during viral infection, viral surface proteins bind to host cell receptors to promote internalization of the viral genome. Inhibitors of the interaction between a viral surface protein and host cell receptors may be used to prevent viral infection or spread of such an infection to other host cells. Elucidation of protein-protein interactions have also led to development of immunotherapies and antibodies, which has been useful in the treatment of cancer. Accordingly, efficient methods of identifying peptide binders of target proteins are warranted.

### SUMMARY OF THE INVENTION

Aspects of the present disclosure relate to peptides binders of target proteins that may be useful in the treatment of disease and methods of identifying such peptides. Further aspects of the present disclosure provide non-naturally occurring peptides. In some embodiments, a non-naturally occurring peptide comprise:
(A) AACX₁X₂X₃X₄X₅X₆MPPX₇X₈X₉X₁₀X₁₁X₁₂C (SEQ ID NO: 1) (scaffold L1), wherein:
   (i) X₆ and X₇ are each the amino acid S or T;
   (ii) X₁-X₅ and X₈-X₁₂ are each any amino acid; and
   (iii) the peptide comprises a thioether bridge that links C at position 3 in to S or T at position 9 in SEQ ID NO: 1 and a thioether bridge that links S or T at position 13 to C at position 19 in SEQ ID NO: 1;
(B) X₁PX₂TTX₃X₄TX₅X₆X₇EX₈X₉DX₁₀DEX₁₁X₁₂X₁₃ (SEQ ID NO: 2) (scaffold L2), wherein:
   (i) X₂ is the amino acid H, Q, N, K, D, or E;
   (ii) X₆ is the amino acid F, L, S, I, M, T, V, or A;
   (iii) X₇ is the amino acid F, L, I, or V;
   (iv) X₁, X₃-X₅ and X₈-X₁₃ are each any amino acid; and
   (v) the peptide comprises an ester bridge that links T at position 5 of SEQ ID NO: 2 to D at position 15 of SEQ ID NO: 2 and an ester bridge that links T at position 8 of SEQ ID NO: 2 to E at position 12 of SEQ ID NO: 2;
(C) X₁CX₂X₃X₄X₅X₆CX₇X₈X₉X₁₀X₁₁ (SEQ ID NO: 3) (scaffold L3), wherein:
   (i) X₅ and X₁₀ are each the amino acid D or E;
   (ii) X₁-X₄, X₆-X₉, and X₁₁ are each any amino acid; and
   (iii) the peptide comprises a thioether bridge that links C at position 2 to D or E at position 6 of SEQ ID NO: 3 and a thioether bridge that links C at position 8 to D or E at position 12 of SEQ ID NO: 3;
(D) X₁CX₂X₃CX₄X₅X₆X₇X₈X₉ (SEQ ID NO: 4) (scaffold L4), wherein:
   (i) X₄ and X₇ are each the amino acid D or E;
   (ii) X₁-X₃, X₅-X₆, and X₈-X₉ are each any amino acid; and
   (iii) the peptide comprises a thioether bridge that links C at position 2 to D or E at position 6 of SEQ ID NO: 4 and a thioether bridge that links C at position 5 to D or E at position 9 of SEQ ID NO: 4; and/or
(E) X₁CX₂X₃X₄X₅X₆CX₇X₈CX₉X₁₀X₁₁X₁₂X₁₃ (SEQ ID NO: 5), wherein:
   (i) X₅, X₉, and X₁₂ are each the amino acid D or E;
   (ii) X₁-X₄, X₆-X₈, X₁₀-X₁₁, and X₁₃ are each any amino acid; and
   (iii) the peptide comprises a thioether bridge that links the C at position 2 to D or E at position 6 of SEQ ID NO: 5, a thioether bridge that links C at position 8 of SEQ ID NO: 5 with D or E at position 12 of SEQ ID NO: 5, and a thioether bridge that links C at position 11 with D or E at position 15 of SEQ ID NO: 5.

In some embodiments, the non-naturally occurring peptide comprises scaffold L5 and a sequence selected from SEQ ID NOS: 6-16; and/or scaffold L3 and a sequence selected from SEQ ID NOs: 17-25. In some embodiments, the non-naturally occurring peptide comprises scaffold L3 and SEQ ID NO: 24.

Further aspects of the present disclosure provide host cells comprising a heterologous nucleic acid encoding any of the non-naturally occurring peptides disclosed herein.

In some embodiments, the heterologous nucleic acid further encodes SEQ ID NO: 46.

In some embodiments, the heterologous nucleic acid comprises any one of SEQ ID NOs: 47-66.

Further aspects of the present disclosure provide:
(a) a first fusion protein comprising (i) a first fragment of a transcription factor, (ii) a first split intein, and (iii) a target protein;
(b) a second fusion protein comprising (i) a candidate peptide, (ii) a second split intein, and (iii) a second fragment of the transcription factor; wherein the first split intein and second split intein are complementary fragments; and
(c) an inducible promoter operably linked to at least one reporter gene, wherein the transcription factor induces transcription of the at least one reporter gene when the transcription factor is present as a full-length transcription factor.

In some embodiments,
(A) in (a), the first fusion protein comprises (i)-(iii) linked sequentially from the N-terminus to the C-terminus, the first fragment is a N-terminal fragment of the transcription factor and the first split intein is a N-terminal split intein; and
(B) in (b), (i)-(iii) are linked sequentially from the N-terminus to the C-terminus, wherein the second split intein is a C-terminal split intein, and the second fragment is a C-terminal fragment of the transcription factor; or
(C) in (a), from the N-terminus to the C-terminus, the first fusion protein comprises (iii) linked to (ii) linked to (i), wherein the first fragment is a C-terminal fragment of the transcription factor and the first split intein is a C-terminal split intein; and
(D) in (b), from the N-terminus to the C-terminus, the second fusion protein comprises (iii) linked to (ii) linked to (i), wherein the second split intein is a N-terminal split intein and the second fragment is a N-terminal fragment of the transcription factor.

In some embodiments, the cell is a eukaryotic or prokaryotic cell, optionally wherein the prokaryotic cell is a bacterial cell.

In some embodiments, the transcription factor is a sigma factor (a factor).

In some embodiments, the first fusion protein is encoded by a first heterologous nucleic acid and the second fusion is encoded by a second heterologous nucleic acid.

In some embodiments, the candidate peptide comprises a sequence selected from SEQ ID NOs: 6-25 or comprises the non-naturally occurring peptide of any one of claims 1 or 2, optionally wherein the candidate peptide further comprises SEQ ID NO: 46.

In some embodiments, the at least one reporter gene encodes a positive selection marker, a negative selection marker, and/or a fluorescent protein, optionally wherein the positive selection marker is an antibiotic resistance gene, optionally wherein the antibiotic resistance gene is chloramphenicol acetyltransferase (cat), optionally wherein the negative selection marker is the herpes simplex virus-thymidine kinase (hsvtk) gene.

In some embodiments, the inducible promoter is an ECF promoter.

In some embodiments, the target protein comprises viral receptor binding domain (RBD) of the SARS-CoV-2 spike protein.

In some embodiments, the RBD comprises SEQ ID NO: 71.

In some embodiments, the host cells further comprises one or more enzymes selected from ProcM, LynD, TgnB, or PapB, optionally wherein the host cell comprises a heterologous nucleic acid encoding the enzyme, optionally wherein the heterologous nucleic acid encoding the enzyme comprises an inducible promoter.

Further aspects of the disclosure provide methods of identifying a peptide that binds a target protein. In some embodiments, the methods comprise culturing any of the host cells disclosed herein and detecting transcription of the at least one reporter gene, thereby identifying the candidate peptide as being capable of binding to the target protein.

In some embodiments, the methods comprise incubating in a reaction vessel:
(a) a first fusion protein comprising (i) a first fragment of a transcription factor, (ii) a first split intein, and (iii) a target protein;
(b) a second fusion protein comprising (i) a candidate peptide, (ii) a second split intein, and (iii) a second fragment of the transcription factor; wherein the first and second split inteins belong to the same intein; and
(c) an inducible promoter operably linked to at least one reporter gene, wherein the transcription factor induces transcription of the at least one reporter gene when the transcription factor is present as a full-length transcription factor, and

detecting transcription of the reporter gene, thereby identifying the candidate peptide as being capable of binding to the target protein.

Further aspects of the present disclosure provide methods of treating a subject having or suspected of having a SARS-CoV-2 infection comprising administering an effective amount of any of the non-naturally occurring peptides disclosed herein.

In some embodiments, the method comprises repeating the method with a plurality of candidate peptides.

In some embodiments, culturing comprises positive and/or negative selection of the host cell.

In some embodiments, the method further comprises sequencing.

Further aspects of the disclosure provide libraries of peptides. In some embodiments, a library compress a plurality of peptides, wherein each peptide of the plurality of peptides has a length of n amino acids and has an amino acid sequence defined by a motif X₁X₂X₃X₄ . . . Xₙ, wherein n is 5-100, wherein each of X₁-Xₙ is independently selected from a group consisting of up to 20 amino acids and at least one of X₁-Xₙ within each peptide is an amino acid selected from a group consisting of 19 or fewer amino acids, and wherein the motif X₁X₂X₃X₄ . . . Xₙ is determined to be susceptible to post-translational modification by at least 2 distinct protein modification enzymes.

In some embodiments, less than 80% of the plurality of peptides are capable of being fully modified by the at least 2 distinct protein modification enzymes.

In some embodiments, at least one of X₁-Xₙ is defined to be a single amino acid.

According to some aspects of the disclosure, compositions comprising host cells are provided. In some embodiments, a composition comprises a plurality of host cells, each host cell of the plurality comprising a peptide of a library disclosed herein, wherein the peptide comprised by each host cell is independent of the peptide comprised by each other host cell. In some embodiments, the composition comprises each peptide of the plurality of peptides. In some embodiments, the host cells are bacterial cells. In some embodiments, the peptide is encoded by a first nucleic acid sequence in the host cell. In some embodiments, each host cell further comprises at least one protein modifying enzyme. In some embodiments, the at least one protein modifying enzyme is encoded by a second nucleic acid sequence in the host cell.

Further aspects of the disclosure provide methods of designing amino acid motifs. In some embodiments, a method of designing an amino acid motif comprises:

(i) selecting one or more protein modifying enzymes;

(ii) identifying a recognition site (RS) sequence for each of the one or more protein modifying enzymes;

(iii) constructing a plurality of nucleic acid molecules, each nucleic acid molecule encoding a leader amino acid sequence comprising the RS sequence for each of the one or more protein modifying enzymes;

(iv) assigning a score to each of the plurality of nucleic acid molecules; and

(v) selecting one of the plurality of nucleic acid molecules based on step (iv),

to design the amino acid motif, wherein each RS sequence facilitates interaction of the corresponding protein modifying enzyme to a peptide defined by the amino acid motif, and wherein the leader amino acid sequence encoded by the nucleic acid molecule selected in step (v) is comprised within each peptide defined by the amino acid motif.

In some embodiments, each peptide defined by the amino acid motif further comprises a core sequence.

In some embodiments, the core sequence comprises one or more amino acids susceptible to modification by the one or more protein modifying enzymes.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein. For purposes of clarity, not every component may be labeled in every drawing. It is to be understood that the data illustrated in the drawings in no way limit the scope of the disclosure. In the drawings:

FIG. 1 shows a schematic of the split intein-based selection. Positive selection is effected by the expression of a chloramphenicol acetyltransferase (cat); negative selection effected by expression of herpes simplex virus thymidine kinase (hsvtk). Both effectors are expressed as fluorescent fusion proteins to facilitate population analysis/sorting by cytometry/FACS.

FIGS. 2A-2G show positive and negative transcriptional selection systems. FIG. 2A shows a genetic representation of selection operon comprising two fused proteins: superfolder-GFP fused to chloramphenicol acetyl transferase (sfGFP-CAT) and Herpes Simplex Virus thymidine kinase fused to mScarlet-I (HSVtk-mScarlet-I). FIG. 2B shows a schematic representation of a positive selection conducted with chloramphenicol (Cm). Only cells that have expressed sfGFP-CAT will be able to grow in the presence of Cm. FIG. 2C shows a schematic representation of a negative selection conducted with the nucleoside analog dP. Cells that have expressed HSVtk-mScarlet-I will not survive in the presence of dP. FIG. 2D shows a demonstration of titratable positive selection growth rescue dependence on expression of sfGFP-CAT (quantified through GFP relative expression units (REUs)) and the applied concentrations of Cm. FIG. 2E shows a demonstration of titratable negative selection growth inhibition dependence on expression of HSVtk-mScarlet-I (quantified through RFP REUs) and the applied concentrations of dP. FIG. 2F shows a schematic representation of the relationship between GFP REUs and the expression of sfGFP-CAT. Since sfGFP is translationally-fused to CAT, expression of CAT can be directly monitored and quantified by observing cellular fluorescence in the green channel. FIG. 2G shows a schematic representation of the relationship between RFP REUs and the expression of HSVtk-mScarlet-I. Since mScarlet-I is translationally-fused to HSVtk, expression of HSVtk can be directly monitored and quantified by observing cellular fluorescence in the red channel.

FIG. 3 shows a design of RiPP libraries for selections. The structures on the right-hand side correspond to scaffolds L1-L5 (SEQ ID NOs: 1-5). Scaffold L1 is a non-limiting example of a lanthipeptide scaffold. Scaffold L2 is a non-limiting example of a microviridin scaffold. Scaffolds L3-L5 are non-limiting examples of a ranthipeptide scaffold.

FIG. 4 shows a schematic of selection methods for initial campaign.

FIGS. 5A-5C show selections profiling of PapB library 1 (L3). FIG. 5A shows ring topology and amino acid degeneracy of library. FIG. 5B shows iterative selection stringencies are assigned an “sl” (selection) designation. FIG. 5C shows cytometry profiling of populations post-selection (from left to right, after a first round, second round, and third round of selection, respectively). First round of selection lead to escape mutants as evidenced by high REU values without induction. Later rounds demonstrate ideal population distributions.

FIGS. 6A-6B include data showing PapB library 1 (L3) hits. FIG. 6A shows PapB library 1 (L3) hits comprising >1% of the final population that were sequentially enriched throughout rounds of selection. FIG. 6B shows amino acid sequences and predicted cyclization topologies.

FIGS. 7A-7C show selection profiling of PapB library 3 (L5). FIG. 7A shows ring topology and amino acid degeneracy of library. FIG. 7B shows iterative selection stringencies are assigned a “sl” (selection) designation. FIG. 7C shows cytometry profiling of populations post-selection (from left to right, after a first round, second round, and third round of selection, respectively). First round of selection lead to escape mutants as evidenced by high REU values without induction. Later rounds demonstrate ideal population distributions.

FIGS. 8A-8B include data showing PapB library 3 (L5) hits. FIG. 8A shows PapB library 3 (L5) hits comprising >1% of the final population that were sequentially enriched throughout rounds of selection. FIG. 8B shows amino acid sequences and predicted cyclization topologies.

FIGS. 9A-9C show individual confirmation assays of selection hits. FIG. 9A shows REU values of 20 hits against the RBD-intein. FIG. 9B shows fold specificity of hits against the RBD. Fold specificity is defined as (REU-RBD)/(REU-Mdm2) where REU-RBD is the REU value of peptide against RBD-intein as target and REU-Mdm2 is the REU value of peptide against Mdm2-intein as target. FIG. 9C shows amino acid sequence and predicted topology of primary hit from pilot selection.

FIGS. 10A-10D show leader-dependent enzyme design constraints. FIG. 10A illustrates design constraints for leader-dependent modifying enzymes. FIG. 10B shows a flowchart for data acquisition and analysis for determining recognition sites. FIG. 10C shows the alanine scan variants for determining important residues in the TgnA precursor peptide. Residues replaced with alanine are indicated by the thicker portions in the bottom left sequence schematics. ΔΔGᵢ for each position is shown above the wild-type sequence. FIG. 10D shows the recognition site constraints for each of the leader-dependent enzymes. Secondary structure is shown above each peptide sequence. The recognition site for each of the leader-dependent enzymes is outlined by a box. Residues that had high ΔΔGᵢ scores but were not included in the recognition sites are labeled with an asterisk in the PlpA2 schematic. Residues that were included in the recognition site to maintain secondary structure are labeled with a triangle in the TruE schematic. Scatter plots show spacing variants for each enzyme. The fit line is based on Equation 3 from Example 3 with fit parameters listed in Table 6. FIG. 10E shows insertion and spacing variants tested for TgnA. Deletions were at the site indicated by the notch in each variant schematic (of the top 10 shown), and insertions are indicated by the thicker portion on the rightmost end of the last three variants. Insertion/deletion size is listed for each variant, alongside fractional modification. The thicker portion at the lefthand side of each variant schematic represent the recognition site.

FIGS. 11A-11C show the core motifs required for enzyme modification. FIG. 11A shows single mutant variant data for TgnA variants. The wild-type sequence is listed at the top, and each row represents a different variant with only mutated residue shown. The dashed line separates poorly modified residues (<50% of wild-type) from well modified residues (>50% of wild-type). The sequences above the dashed line were used to build the motif. FIG. 11B shows leader-dependent enzyme motifs. FIG. 11C shows leader-independent enzyme motifs. In FIGS. 11B and 11C, for each motif, the enzyme name is in bold and shown above the peptide name. Amino acids shown below the boxed wild-type sequence were observed and well-modified. Amino acids shown above the boxed sequence were not tolerated. Unobserved amino acids are not shown, except for positions labeled with a star or dagger. The core position of the first motif residue is labeled above the position, with the +1 sites additionally annotated in PaaP and LynD. Chemical modifications are shown on the modified residue(s) which are bolded. Positions that are allowed to be any amino acid are noted with a star, and a dagger indicates that the position is allowed to be any residue except cysteine.

FIGS. 12A-12D show automated design of hybrid core motifs and multi-modification of core peptide. FIG. 12A illustrates a design algorithm that combines user input of desired modifications and their positions, with demonstrated design showing combination of PlpXY, LynD, and ThcoK constraints. FIG. 12B illustrates an expression construct, showing inducer control of precursor peptide and modifying enzymes, modification of the precursor peptide, and cleavage to generate the final molecule.

FIGS. 13A-13J illustrate the split intein system for in vivo detection of protein-protein interactions. FIG. 13A shows a schematic of the binding interaction between the SARS-CoV-2 spike protein and the human ACE2 receptor. The receptor binding domain (RBD) of Spike protein is darkened. FIG. 13B shows a structural representation of the spike-ACE2 interaction. Only the spike RBD and the two N-terminal helices of ACE2 are shown. PDB ID: 6M17 97. FIG. 13C shows a schematic for the detection of a binding event between a RiPP and target. FIG. 13D illustrates inducible interaction-mediated splicing. The median fluorescence is shown as a function of the expression of the two halves of the sensor proteins. The induction of PMI-and Mdm2-driven association (left) or split intein alone (right) are shown. FIG. 13E shows the specificity calculated using the data in FIG. 13D: (Mdm2*−PMI)/(no bait-no peptide). The white dot in the lower right quadrant marks the highest fold-change in expression. FIG. 13F shows the fluorescence measured from the circuit containing a binding pair (Mdm2:PMI) and non-binding pairs. The 3O6-AHL concentration for all inductions was 1 μM. Three replicates and the mean values are shown. FIG. 13G shows a schematic of the RiPP-containing half of the split intein system. The modified core residue positions are shaded, and the RS for the modifying enzyme is shown within the leader. FIG. 13H shows the structure of the wild-type PapA modified peptide with a dashed box around the region used to design the peptide library. FIG. 13I shows a library sequence weblogo for the 9 unmodified library variants observed. FIG. 13J shows a library sequence weblogo for the 5 modified library variants observed.

FIGS. 14A-14F show a selection system to identify RBD-binding RiPPs. FIG. 14A shows a genetic circuit diagram for the RBD-binding RiPP selection system that is distributed across three plasmids and two genomic regions. Three small molecules: 3OC6-AHL, aTc, and cumate control the expression of the RiPP peptide, modifying enzyme 1, and modifying enzyme 2 (if present), respectively. FIG. 14B shows a schematic of selection output under two conditions: with an RBD-binding RiPP (top) and without an RBD-binding RiPP (bottom). Binding is shown to result in the production of chloramphenicol acetyltransferase (CAT; squares), such that bacteria in which an RBD-binding RiPP is expressed are selected based on chloramphenicol (Cm) resistance. FIG. 14C shows an overview of the positive of selection applied. Selection rounds were conducted in the presence of RiPP peptide and modifying enzymes and used increasing Cm concentrations for increased stringency. FIG. 14D shows a core scaffold for the pap2c library (lbAMK-103). Predicted macrocyles are indicated by brackets above constrained residues and “X” residues correspond to NNK translated amino acids. FIG. 14E show cytometry distributions for positive selections on the pap2c library beginning with no selection, round 2, and round 3 of positive selections (0, 800, and 1200 μM Cm, respectively). Fluorescence of the sfGFP fused to CAT is reported. FIG. 14F shows the measured fluorescence induced by an RBD-specific hit isolated from genetic selection. The RBD-binding RiPP was used as peptide against the non-specific bait (Mdm2*) and specific bait (RBD). The means were calculated from median fluorescence intensity of three replicates.

FIGS. 15A-15F show characterization of AMK-1057, a cyclic peptide that binds human-derived Spike RBD in vitro. FIG. 15A show a schematic of the peptide expression, modification, cleavage and purification steps. TEV cleavage removes the SUMO tag and HPLC purification produces the final product. Following TEV cleavage, a single G from the leader is left at the N-terminus of the product peptide. FIG. 15B shows high-resolution MS of unmodified AMK-1057 (top trace) and singly modified AMK-1057 (bottom trace). FIG. 15C shows high-resolution MS/MS of modified AMK-1057 and fragment mapping to the amino acid sequence. Numbered peaks correspond to fragment ions observed and represented as lettered amino acids next to MS/MS spectrum. FIG. 15D shows structural annotation of AMK-1057. FIG. 15E shows binding of purified, modified AMK-1057 to Spike RBD296-531 derived from a human cell line. Vertical dotted line indicates the start of the dissociation phase of the measurements. FIG. 15F shows binding of purified, unmodified AMK-1057 to human-derived Spike RBD. Vertical dotted line indicates the start of the dissociation phase of the measurements.

FIGS. 16A-16B illustrates cell competition for ACE2 binding by AMK-1057:RBD complex. FIG. 16A shows a schematic of ACE2 receptor binding inhibition by binding of AMK-1057 to RBD. FIG. 16B shows cytometry distributions for positive control (top trace; cells incubated with RBD only), negative control (bottom trace; cells incubated with vehicle), and cells incubated with RBD pre-incubated with 5 μM or 50 μM AMK-1057. Fluorescence signal represents fluorescence from labeled RBD.

FIGS. 17A-17B show optimization of binding affinity by tuning peptide expression. FIG. 17A shows comparisons of measured peptide binding to an on-target bait or an off-target bait under conditions of low peptide expression. FIG. 17B shows comparisons of measured peptide binding to an on-target bait or an off-target bait under conditions of high peptide expression. The results demonstrate that tuning expression allows characterization of peptide binding.

FIGS. 18A-18B show directed evolution of AMK-1057. FIG. 18A shows variant enrichment for single amino acid substitutions. Heatmap shows variant enrichment relative to the parent peptide. Arrows indicate selected core positions with substitutions that yielded positive enrichment. FIG. 18B show cytometry data for consensus variants containing up to 3 amino acid substitutions per variant, at the three positions indicated by arrows in FIG. 18A. The labeled peptide name indicates the respective amino acids at each of the three selected positions (e.g., “IVE” indicates that the indicated core amino acids were IVE rather than AVE in the parent peptide).

FIGS. 19A-19C show competition of AMK-1057 binding to RBD, measured via bio-layer interferometry. FIG. 19A shows AMK-1057 interferometry results measured with RBD in the presence of B38 antibody that does not overlap with the RBD ACE2 binding site. FIG. 19B shows AMK-1057 interferometry results measured with RBD in the presence of CR3022 antibody which overlaps with the RBD ACE2 binding site. FIG. 19C shows AMK-1057 interferometry results measured with RBD and AMK-1057 alone.

FIG. 20 shows an outline of native RiPP biosynthesis and export.

FIG. 21 shows the data mining strategy used to identify candidate peptide clusters. HMP microbial genomes were scanned for RiPP BGCs using AntiSMASH 4.0, a sequence similarity network generated for BGCs using BiG-SCAPE, and visualized using Cytoscape.

FIG. 22 shows a sequence similarity network of human microbiome RiPP BGCs. antiSMASH 4.0 was used to identify BGCs from 2,229 HMP genome sequences. 2,233 RiPP BGCs were clustered using BiG-SCAPE and visualized with Cytoscape. Nodes represent individual clusters shaded according to biosynthetic class. BGC nodes with similar cluster architecture are attached by edges.

FIGS. 23A-23E show a platform for large-scale RiPP BGC mining from sequence data. FIG. 23A shows the typical organization and native processing of a lanthipeptide BGC (the BGC and cartoon structure of nisin is shown). A ribosomally produced precursor peptide (RiPP) is dehydrated, cyclized, and cleaved to produce a mature antimicrobial cyclic peptide, nisin. lanA, precursor peptide; lanBC, lanthionine synthetase; lanT, transport; lanIFEG, immunity; lanP, leader peptide cleavage; lanRK, transcriptional regulation. FIG. 23B shows the typical organization and native processing of a lasso peptide BGC (the BGC and cartoon structure of microcin J25 is shown). A RiPP is cleaved and cyclized to produce a mature antimicrobial cyclic peptide, microcin J25. lasA, precursor peptide; lasBC, lasso peptide synthetase; last, transporter. FIG. 23C shows an engineered peptide expression system for lanthipeptides. An N-terminal hexa-histidine-SUMO fusion tag (HS-tag) followed by a protease site and precursor peptide allows for stabilized expression of putative lanthipeptide peptide sequences. Expression with putative modifying enzymes followed by affinity purification and in vitro proteolysis yields mature, processed peptide for assaying biological activity. FIG. 23D shows an engineered peptide expression system for lasso peptides. A C-terminal HS-tag was used instead of N-terminal to allow for leader peptide cleavage as part of biosynthesis. FIG. 23E shows a schematic for screening of engineered peptides. In the screening method, DNA sequences for putative precursor peptides and core biosynthetic enzymes are synthesized on medium copy plasmid backbones and transformed into an expression strain of E. coli in 96-well density. Expression, purification, processing, LC-MS analysis, and biological activity testing can all be done in 96-well plates.

FIG. 24 shows a taxonomic tree of lanthipeptide and lasso peptide producing organisms selected for heterologous expression.

FIG. 25 shows an example RiPP BGC and basic two-plasmid expression system for heterologous expression. HS, hexa-histidine-SUMO fusion tag.

FIG. 26 shows LC-MS traces corresponding to the BGC cluster shown in FIG. 25. The larger trace shows total ion chromatogram (TIC) for the peptide expressed alone or with modifying enzyme. The inset trace shows mass shifts from mass spectra taken from TIC peaks. Mass loss corresponds to multiple dehydrations indicating enzymatic modification.

FIG. 27 shows the results of tandem MS and HSEE analysis to annotate peptide structure. Single letters correspond to amino acids; lowercase b indicates dehydrobutyrine.

FIGS. 28A-28E show results of analysis of data mined for putative tailoring enzymes using the Marionette expression system, which enables high-throughput assaying of such enzymes. FIG. 28A shows the relative abundance of pfam domain occurrence in genetic proximity to lanBC modifying enzymes involved in type I lanthipeptide biosynthesis. FIG. 28B shows the relative abundance of pfam domain occurrence in genetic proximity to lanM modifying enzymes involved in type II lanthipeptide biosynthesis. FIG. 28C shows the relative abundance of pfam domain occurrence in genetic proximity to lanM modifying enzymes involved in type III lanthipeptide biosynthesis. FIG. 28D shows the relative abundance of pfam domain occurrence in genetic proximity to lasBC modifying enzymes involved in lasso peptide biosynthesis. FIG. 28E shows a schematic of the strategy for mining tailoring enzymes using the Marionette collection of orthogonal inducible promoters. In the screening strategy, putative precursor peptides (lanA), core modifying enzymes (lanBC), and putative tailoring enzymes (lanH1-3) are synthesized on individual plasmids and a one-pot type IIs assembly reaction generates a single modifying enzyme plasmid for use in co-expression platform. Each putative enzyme is under control of a separate inducer, allowing for systematic interrogation of function.

FIG. 29 shows RiPPs mined from diverse strains of the human microbiome. Peptides are organized by producing organism niche. Gene clusters are highlighted for open reading frames that were synthesized and heterologously expressed. Arrows show putative peptides, putative lanthionine synthetases, and putative tailoring enzymes. TIC traces are shown to the right of clusters with shading indicating eluted peptides. The peptide structures shown are annotated through tandem MS and HSEE.

FIG. 30 shows lasso peptides identified by mining the human microbiome.

FIGS. 31A-31D show identified candidate lanthipeptide tailoring enzymes. FIGS. 31A, 31B, and 31C show source BGCs and producing organisms followed by TIC traces+/−expression of tailoring enzymes and MS of largest peak. Precursor peptides (lanA, lanA1, lanA2) and modifying enzymes (lanM, lanB, lanC) are displayed, as are putative tailoring enzymes (lanH1, lanH2, and lanH3). BLAST was used to assign hypothetical tailoring enzyme annotations. In FIG. 31A, a flavodoxin-containing protein causes the production of a peak difficult to resolve via MS. In FIG. 31B, combined expression of OsmC family peroxiredoxin and truncated N-terminus of a lanM results in generation of a peak with a mass shift of +535.4 Da. In FIG. 31C, combined expression of a hut-D-like cupin, SM1 toxin immunity, and KptA-like protein resulted in generation of a peak with a mass shift of −533.2 Da. FIG. 31D shows TIC traces for expression of peptide and different tailoring enzymes from the M. odoratimimus BGC shown in FIG. 31C. These results demonstrate that KptA-like protein is required for modification of the peptide. The modified peptide corresponds to mass observed in FIG. 31C.

FIGS. 32A-32D show phylogenetic analysis of lanthipeptide producers. FIG. 32A shows a phylogenetic tree of all lanthipeptide producers. Organisms with BGCs that were successfully expressed in E. coli and detected are shaded. The tree was generated using NCBI taxonomic identifiers. FIG. 32B shows type I lanthipeptide synthetase (LanBC) modifications, which use glutamyl-tRNA (tRNA^{Glu}) to glutamylate Serine/Threonine residues for dehydration and subsequent cyclization. FIG. 32C shows type II/III lanthipeptide synthetase (LanM/K) modifications, which use ATP to phosphorylate Serine/Threonine residues for dehydration and subsequent cyclization. FIG. 32D shows a phylogenetic tree generated using tRNA^{Glu} sequences from type I lanthipeptide producers investigated herein. Organisms with BGCs that were successfully expressed in E. coli and detected are shaded.

FIGS. 33A-33B show results of screens for RiPP antimicrobial activity. FIG. 33A shows select images of zones of inhibition from disc-diffusion assays of purified lanthipeptides. For each row of images, the compound ID, microbiome niche, and producing organism are listed. Indicator organisms used are organized in columns. Circles in each image highlight zones of inhibition observed. FIG. 33B shows a heat map displaying residual growth in the presence of a set amount of SPE-purified RiPP. Residual growth was calculated for all indicator organisms as a ratio of OD600 measured in comparison to growth and sterility controls. These data demonstrate that lanthipeptides mined from the human microbiome have unique antimicrobial fingerprints.

FIG. 34 shows heat maps displaying residual growth in the presence of serial dilutions of antimicrobial lanthipeptides. Compound ID, microbiome niche, and producing organisms are displayed above each antimicrobial profile. Each row corresponds to the indicator organism grown in the presence of a 2-fold serial dilution of SPE-purified peptide. These results demonstrate that lanthipeptides mined from the human microbiome are active against MDR pathogens.

FIGS. 35A-35F show sequence-activity relationships of selected peptides. FIG. 35A shows cluster-associated Streptococcus-derived lanthipeptide core sequences. Amino acid similarity is annotated by extent of blue shading and consensus identity displayed above core sequences. Alignment was generated using the Geneious global alignment tool with free end gaps and a Blosum62 cost matrix. FIG. 35B shows the antimicrobial profiles of selected lanthipeptides against human microbiome bacterial strains. FIG. 35C shows the predicted structure of the related lanthipeptides from hypothetical structural annotation. Modified amino acids are shown with thick outlines. ‘b’ indicates dehydrobutyrine and ‘a’ indicates dehydroalanine. FIG. 35D shows amino acid sequence alignment of Rothia-derived cluster-associated lasso peptide core sequences. Amino acid similarity is annotated by darkness of shading, and the consensus identity sequence is displayed above the cored sequences. Alignment was generated using the Geneious global alignment tool with free end gaps and a Blosum62 cost matrix. FIG. 35E shows the antimicrobial profiles of selected lasso peptides against human microbiome bacterial strains. FIG. 35F shows the predicted structure of the related lasso peptides from hypothetical structural annotation. Modified amino acids are shown with thick outlines.

FIG. 36 shows peptide motifs for modification by LynD, PlpXY, PalS, PadeK, PaaA, ThcoK, TgnB, LasF, and EpiD enzymes. In each motif, the amino acid(s) modified by each respective enzyme are bolded. The boxed core peptide sequence shows the parental sequence, and the amino acids annotated below each position show the options that are allowable for each modification enzyme. The left-most boxed amino acids in the LynD (LAELSEEAL (SEQ ID NO: 84)), PlpXY (LNEEELEAIAG (SEQ ID NO: 85)), PaaA (SQRISAIT (SEQ ID NO: 86)), and TgnB (PYIAKYV (SEQ ID NO: 87)) motifs show the leader recognition site (RS) sequences, and the distance ranges annotated above the LynD, PlpXY, and TgnB motifs indicate the limitation on available distances between the RS and the amino acid to be modified.

FIGS. 37A-37F show identification of a peptide motif to be modified by three distinct enzymes (two leader-dependent enzymes and one tailoring enzyme). FIG. 37A shows an example schematic of a peptide motif (leader+core) with three modifying enzymes. FIG. 37B shows an example motif with three particular modifications incorporated by three distinct enzymes (top) and the chemical structure of an example peptide with those modifications. FIG. 37C shows the peptide motif generated by combining the distinct motif restrictions for LynD, PlpXY, and ThcoK. In the right motif, the amino acids shown below each position in the peptide schematic indicate the allowable amino acids at each given position based on the combination of three enzyme restrictions. FIG. 37D shows a schematic of the screening method for identifying the leader sequence incorporating the LynD and PlpXY recognition sites (RSs) and the score calculated for each possible leader. FIG. 37E shows the identified peptide motif (leader+core) based on the combination of LynD, PlpXY, and ThcoK sequence restrictions. FIG. 37F shows 11 peptides isolated from screening the degenerate library built based on the motif shown in FIG. 37E. Amino acids that did not fall within the motif are shaded.

FIG. 38 shows peptide motifs built for modification by various combinations of distinct modifying enzymes, labeled to the left of each amino acid sequence. The modifications introduced by the specific combination of enzymes are shown on each amino acid sequence.

FIG. 39 shows a schematic of the Small Ubiquitin-like Modifier (SUMO) protein tag (top) used in an approach to stabilize ribosomally synthesized and post-translationally modified peptides (RiPPs), allowing modification of the core peptide sequence by modification enzymes, purification, and isolation of the modified peptide. The RiPP stabilization tag comprises an affinity-tag, a solubilization-tag, and a TEV or thrombin cleavage site, with flexible linkers separating elements. It stabilizes precursor peptides when attached to the N- or C-terminus, and is compatible with many diverse protein-modifying enzymes. Example peptide modifications facilitated thereby are also shown (bottom).

FIGS. 40A-40C show an overview of an expression system for producing modified peptides. FIG. 40A shows a schematic of N-terminal and C-terminal RiPP stabilization tags (RSTs). FIG. 40B shows a two-plasmid system used for expression of the RST-tagged precursor peptide (top) and modifying enzyme (bottom). The peptide-expressing plasmid is IPTG-inducible, and the modifying enzyme is cumate-inducible. FIG. 40C shows a schematic of the subsequent analysis steps following peptide synthesis. Peptides extracted from their host cells are analyzed by LCMS for mass shifts associated with modification. The low molecular weight of the RST allows for easier high confidence analysis of the modification.

FIG. 41 shows stabilization of unmodified peptides from diverse RiPP classes using the SUMO tag. SUMO protein successfully stabilized expression of seven precursor peptides with varying lengths and amino acid compositions, each from a different RiPP family, with two additional peptides stabilized after cleavage (presumably by endogenous E. coli proteases). In comparison, HIS6 tag only successfully showed four minor peptide peaks. Boxes in the microviridin, bottromycin, streptide, pyrroloquinoline quinone, lanthipeptide, thiopeptide, and pheganomycin traces indicate that the given peak was present in sample, absent in the negative control, and had the expected mass; boxes in the sactipeptide and trifolitoxin traces indicate that the given peak was present in sample, absent in the negative control, but did not have the expected mass.

FIGS. 42A-42E show characterization of RST-tagged haloduracin A1 (HalA1) and A2 (HalA2) peptides, demonstrating that RST-tagged peptides can be modified, cleaved, and purified as bioactive molecules. FIG. 42A shows schematics of both RST-tagged HalA1 and HalA2 peptides, which were engineered to have TEV cleavage sites in between the leader and core peptides, with RST_{N} tags. FIG. 42B shows the post-cleavage structures of HalA1 and HalA2. After expression and modification of HalA1 and HalA2, the peptides were purified by LCMS (FIG. 42C) and the SUMO tag and leader peptide were cleaved from the core (FIG. 42D). FIGS. 42C and 42D show LCMS traces for HalA1 (left) and HalA2 (right) during purification and following cleavage, respectively. FIG. 42E shows LC-MS/MS fragmentation spectra of cleaved HalA1 and HalA2, which demonstrate masses that match fragments of the structures shown in FIG. 42B. FIG. 42F shows the results of treatment of B. subtilis reporter strain with HalA1 and HalA2. The results demonstrate that HalA1 and HalA2 individually had minimal antibacterial activity, but both haloduracins together successfully inhibited bacterial growth of B. subtilis reporter strain.

FIG. 43 shows a bar chart of successful peptide/modifying enzyme combinations. Peptide plasmid number and gene name, modifying enzyme plasmid number and gene name, and replicate extract numbers are listed alongside fraction modified in TB (dark grey) and LB (light grey) medias. Dashed line demarcates 50% modification (half of peptide modified).

FIGS. 44A-44D show maps of plasmids used in Example 3. FIG. 44A shows N-term SUMO Backbone 2. FIG. 44B shows N-term SUMO Backbone 3, which is the same as N-term SUMO Backbone 2 but with flanking Bsa1 restriction sites around the peptide operon. FIG. 44C shows Cumate Modifying Enzyme Backbone. FIG. 44D shows Multi-Enzyme Backbone.

FIGS. 45A-45C show a multiply modified peptide library. FIG. 45A shows a hybrid motif combining the PlpXY, LynD, and ThcoK motifs, with modified positions bolded and showing modification where possible. The bolded tyrosine is excised in the modification process, but still shown in this motif. FIG. 45B shows the peptide sequence that was built, with degenerate nucleotide sequences shown above the peptide structure for each amino acid position, and the resulting amino acids encoded. FIG. 45C shows a set of peptide sequences that were isolated from the library. Amino acid residues that do not match the hybrid motif (shown in FIG. 45A) are shown shaded. The non-matching residues were not observed in the original data set, but were not unallowed. The degenerate nucleotide sequences resulted in production of certain peptides having certain amino acids not included in the hybrid motif. The bolded sequence labels (2582, 2583, 2585, and 2587) indicate the peptides that were successfully triply-modified.

FIG. 46 shows baseline fractional modification for modifying enzymes. Leader, core, and follower sequences were used to establish baseline. A SUMO tag is represented by a square at the beginning of each sequence. The modified residues are underlined.

FIGS. 47A-47D show leader and core amino acid sequence screening for TgnB enzyme. FIG. 47A shows results of an alanine scan (top) and deletion/addition scan (bottom) of the leader sequence, with the fraction modified, ratio modified, ΔΔG for each variant peptide and for each position of the leader sequence. FIG. 47B shows results of a peptide variant scan of the core sequence. The top amino acid sequence in bold represents the wild-type core sequence, and subsequent rows show the substituted amino acid(s) in each variant, alongside the fraction modified for each variant. FIG. 47C shows a core sequence amino acid variant tolerance summary, in which the wild-type sequence is boxed, and amino acids below the wild-type sequence represent amino acids tested at each position that were found to be tolerated, and amino acids shown above represent those tested and found to be not tolerated. FIG. 47D shows sequence constraints for the core motif, the recognition sequence, and the distance between the recognition sequence and the amino acid to be modified.

FIGS. 48A-48D show leader and core amino acid sequence screening for PlpXy enzyme. FIG. 48A shows results of an alanine scan of the leader sequence, with the fraction modified, ratio modified, ΔΔG for each variant peptide and for each position of the leader sequence (top) and candidate deletion/addition peptides (bottom). FIG. 48B shows results of a peptide variant scan of the core sequence. The top amino acid sequence in bold represents the wild-type core sequence, and subsequent rows show the substituted amino acid(s) in each variant, alongside the fraction modified for each variant. FIG. 48C shows a core sequence amino acid variant tolerance summary, in which the wild-type sequence is boxed, and amino acids below the wild-type sequence represent amino acids tested at each position that were found to be tolerated, and amino acids shown above represent those tested and found to be not tolerated. FIG. 48D shows sequence constraints for the core motif, the recognition sequence, and the distance between the recognition sequence and the amino acid to be modified.

FIGS. 49A-47C show leader and core amino acid sequence screening for PaaA enzyme. FIG. 49A shows results of an alanine scan (top) and deletion/addition scan (middle and bottom) of the leader sequence, with the fraction modified, ratio modified, ΔΔG for each variant peptide and for each position of the leader sequence. FIG. 49B shows a core sequence amino acid variant tolerance summary, in which the wild-type sequence is boxed, and amino acids below the wild-type sequence represent amino acids tested at each position that were found to be tolerated, and amino acids shown above represent those tested and found to be not tolerated. FIG. 49C shows sequence constraints for the core motif, the recognition sequence, and the distance between the recognition sequence and the amino acid to be modified.

FIGS. 50A-50D show leader and core amino acid sequence screening for LynD enzyme. FIG. 50A shows results of an alanine scan (top) and deletion/addition scan (bottom) of the leader sequence, with the fraction modified, ratio modified, ΔΔG for each variant peptide and for each position of the leader sequence. FIG. 50B shows results of a peptide variant scan of the core sequence. The top amino acid sequence in bold represents the wild-type core sequence, and subsequent rows show the substituted amino acid(s) in each variant, alongside the fraction modified for each variant. FIG. 50C shows a core sequence amino acid variant tolerance summary, in which the wild-type sequence is boxed, and amino acids below the wild-type sequence represent amino acids tested at each position that were found to be tolerated. Positions with sufficient diversity such that they are allowed to be any amino acid are annotated with a star. Positions with sufficient diversity such that they are allowed to be any amino acid except for cysteine are annotated with a dagger. FIG. 50D shows sequence constraints for the core motif, the recognition sequence, and the distance between the recognition sequence and the amino acid to be modified.

FIGS. 51A-51C show core amino acid sequence screening for EpiD enzyme. FIG. 51A shows results of a peptide variant scan of the core sequence. The top amino acid sequence in bold represents the wild-type core sequence, and subsequent rows show the substituted amino acid(s) in each variant, alongside the fraction modified for each variant. FIG. 51B shows a core sequence amino acid variant tolerance summary, in which the wild-type sequence is boxed, and amino acids below the wild-type sequence represent amino acids tested at each position that were found to be tolerated, and amino acids shown above represent those tested and found to be not tolerated. Positions with sufficient diversity such that they are allowed to be any amino acid are annotated with a star. FIG. 51C shows sequence constraints for the core motif.

FIGS. 52A-52C show core amino acid sequence screening for PalS enzyme. FIG. 52A shows results of a peptide variant scan of the core sequence. The top amino acid sequence in bold represents the wild-type core sequence, and subsequent rows show the substituted amino acid(s) in each variant, alongside the fraction modified for each variant. FIG. 52B shows a core sequence amino acid variant tolerance summary, in which the wild-type sequence is boxed, and amino acids below the wild-type sequence represent amino acids tested at each position that were found to be tolerated, and amino acids shown above represent those tested and found to be not tolerated. Positions with sufficient diversity such that they are allowed to be any amino acid are annotated with a star. FIG. 52C shows sequence constraints for the core motif.

FIGS. 53A-53C show core amino acid sequence screening for LasF enzyme. FIG. 53A shows results of a peptide variant scan of the core sequence. The top amino acid sequence in bold represents the wild-type core sequence, and subsequent rows show the substituted amino acid(s) in each variant, alongside the fraction modified for each variant. FIG. 53B shows a core sequence amino acid variant tolerance summary, in which the wild-type sequence is boxed, and amino acids below the wild-type sequence represent amino acids tested at each position that were found to be tolerated, and amino acids shown above represent those tested and found to be not tolerated. FIG. 53C shows sequence constraints for the core motif.

FIGS. 54A-54C show core amino acid sequence screening for PadeK enzyme. FIG. 54A shows results of a peptide variant scan of the core sequence. The top amino acid sequence in bold represents the wild-type core sequence, and subsequent rows show the substituted amino acid(s) in each variant, alongside the fraction modified for each variant. FIG. 54B shows a core sequence amino acid variant tolerance summary, in which the wild-type sequence is boxed, and amino acids below the wild-type sequence represent amino acids tested at each position that were found to be tolerated, and amino acids shown above represent those tested and found to be not tolerated. Positions with sufficient diversity such that they are allowed to be any amino acid are annotated with a star. FIG. 54C shows sequence constraints for the core motif.

FIGS. 55A-55C show core amino acid sequence screening for ThcoK enzyme. FIG. 55A shows results of a peptide variant scan of the core sequence. The top amino acid sequence in bold represents the wild-type core sequence, and subsequent rows show the substituted amino acid(s) in each variant, alongside the fraction modified for each variant. FIG. 55B shows a core sequence amino acid variant tolerance summary, in which the wild-type sequence is boxed, and amino acids below the wild-type sequence represent amino acids tested at each position that were found to be tolerated, and amino acids shown above represent those tested and found to be not tolerated. Positions with sufficient diversity such that they are allowed to be any amino acid are annotated with a star. FIG. 55C shows sequence constraints for the core motif.

FIG. 56 shows weblogos for the leader peptides of leader-dependent enzymes. Blastp results for each of the leaders (plus core and follower for PaaP) were aligned using Cobalt and visualized using Weblogo. Each weblogo was then aligned to the leader sequence used in Example 3. The x-axis corresponds to the position within each leader sequence and the recognition sites are outlined in boxes.

FIG. 58 shows a phylogenetic tree of species from which enzymes were mined. The tree was generated from the organisms listed in Table 13. Species from which functional enzymes were sourced are shown with a star (*).

FIG. 59 shows a summary of select non-limiting RiPP chemical modifications. Each box shows an example structure with the modified residue(s). The amino acids involved in each chemical modification are shown in the lower left corner of each box, for instances in which amino acids are chemically restrictive.

### DETAILED DESCRIPTION OF THE INVENTION

Aspects of the present disclosure provide efficient methods of identifying peptide binders of target proteins using an intein-based system. As shown herein, the method is useful in identifying peptide binders of a target protein, including the viral receptor binding domain (RBD) of spike protein from SARS-CoV-2. In some embodiments, the methods disclosed herein have been used to identify modified peptide binders of RBD. Additional methods disclosed herein provide an efficient means of identifying peptides with particular properties and/or activity, such as biological activity. Libraries of peptides with useful characteristics are also provided, in addition to methods for their preparation and screening.

Without being bound by a particular theory, modified peptide binders have numerous advantages over traditional drug candidates including small molecule compounds and monoclonal antibodies (mABs). For example, small molecule compounds are often poor inhibitors of macromolecular interactions due to the physicochemical constraints of small molecule compounds; small molecule compounds are often not large enough to cover large binding interfaces. While mABs may be capable of occupying a larger binding surface area as compared to small molecule compounds, development of mABs is often slow, often taking about six months to identify a lead mAB against a target protein, have low stability, often require particular routes of administration (e.g., parenteral administration), and may have low cell penetrability. The methods and modified peptides described herein, in some embodiments, overcome many of these limitations. For example, in some embodiments, the peptide binders comprise modifications that increase stability, promote proteolytic resistance, and/or increase solubility.

Furthermore, conventional antibiotics used as drugs target diverse bacteria as part of their mode of action. This “broad-spectrum” activity has benefit in the treatment of life-threatening bacterial infections, as a single agent is able to address a large number of clinical indications. However, this broad-spectrum activity can also disrupt the subject's microbiome, leading to associated complications in health. The methods disclosed herein provide means for identifying peptides with antimicrobial activity, including narrow-spectrum activity. Narrow-spectrum antimicrobial agents are desirable to avoid microbiome disruptions and to mitigate selection pressure for widespread evolution of resistance to antibiotics. Narrow spectrum agents that can selectively remove specific bacteria are useful as both a subject-specific medicine, and as tool compounds to facilitate understanding of and manipulate the microbiome.

In early-stage drug discovery, candidate compounds are typically identified from two sources: natural products (e.g., isolated from natural sources such as plants or microbes) and combinatorial chemistry libraries of synthetic molecules. Inadequacies in ability to synthesize natural product-like molecules, as well as the prohibitive cost of identifying such molecules from nature, limit the ability to develop products (e.g., peptides) with desirable properties. In addition, molecules from combinatorial chemistry libraries lack the structural complexity necessary to identify ideal drug candidates. Engineered RiPPs provide the ability to biosynthesize structurally diverse small molecules (e.g., peptides) for screening and drug discovery.

In some embodiments, the methods disclosed herein allow for efficient methods of identifying candidate drugs against challenging therapeutic targets (e.g., targets that have been referred to as “undruggable”). Several cancer targets including KRAS, MYC, and transcription factors have been labeled as “undruggable targets” due to their large protein-protein interaction interfaces or due to the absence of protein pockets for binding. See, e.g., Whitfield et al., Front. Cell Dev. Biol. 5, 10 (2017) and McCormick et al., Clin. Cancer Res. 21, 1797-1801 (2015). In some embodiments, challenging therapeutic targets include particular microbes (e.g., drug-resistant bacteria, or bacteria of a class or species that is difficult to treat).

### Split Intein-Based Selection

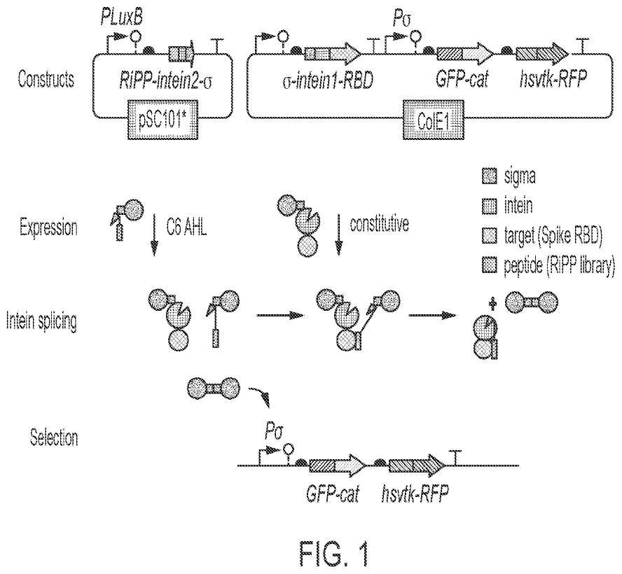

Aspects of the present disclosure provide methods of identifying peptide binders of a target protein using split intein-based selection system. Additional aspects of the present disclosure provide methods of identifying peptides with particular desired properties, such as biological activity using a split intein-based selection system. FIG. 1 provides a non-limiting example of a split intein-based selection system.

An intein is an internal amino acid sequence that is post-translationally autoprocessed. During protein splicing, an intein self-excises from a precursor protein and ligates the flanking N- and C-terminal amino acid sequences (exteins or external protein sequences) via a new peptide bond. For example, a precursor protein may comprise the following configuration: N-extein-intein-C-extein. Following protein splicing, the following peptide is produced: N-extein-C-extein.

The intein, however, may be provided as two separate fragments (split inteins) rather than as contiguous sequence. During trans-splicing, the two fragments of the intein have to associate before protein splicing can occur. As used herein, an N-terminal intein (N-intein) comprises the N-terminal sequence of an intein, while the C-terminal intein (C-intein) comprises the C-terminal sequence of the same intein. When split inteins are used, the N-intein is linked to the C′ terminal end of the N-extein; the C-intein is located at the N′ end of the C-extein. The N-extein and the C-intein may belong to the same protein of interest. For example, the N-extein may comprise an N-terminal fragment of a protein of interest, while the C-intein comprises the C-terminal fragment of the same protein of interest, such that when the N-intein and C-intein associate, a full-length protein of interest is formed. See, e.g., Shah and Muir, Chem Sci. 2014; 5(1):446-461.

Any complementary split intein pair may be used including those known in the art. Non-limiting examples of complementary split inteins include the N-terminal intein NpuDNAE intein N (SEQ ID NO: 68) and the C-terminal intein NpuDNAE intein C (SEQ ID NO: 67). See also, e.g., US20200055900 and Stevens et al., J Am Chem Soc. 2016 Feb. 24; 138(7):2162-5.

In some embodiments, the methods described herein comprise using split inteins. In general, unless indicated otherwise, the split intein-based selection system described herein comprises two fusion proteins and an inducible promoter operably linked to a reporter gene. For example, the first fusion protein generally comprises (i) a first fragment of a transcription factor, (ii) a first split intein, and (iii) a target protein, and the second fusion protein may comprise (i) a candidate peptide, (ii) a second split intein, and (iii) a second fragment of the transcription factor. The first and second split inteins are complementary fragments, such that association of the first split intein with the second split intein promotes trans-splicing and formation of a full-length transcription factor to drive expression from the inducible promoter. As described below, it may also be possible to use the split intein-based system described herein without the need for a reporter gene operably linked to an inducible promoter (e.g., the fragments of the transcription factor may be replaced with fragments of a reporter protein).

In some embodiments, the first fusion protein comprises (i) a first fragment of a transcription factor, (ii) a first split intein, and (iii) a target protein linked sequentially from the N-terminus to the C-terminus, in which the first fragment is an N-terminal fragment of the transcription factor and the first split intein is an N-terminal split intein; and the second fusion comprises: (i) a candidate peptide, (ii) a second split intein, and (iii) a second fragment of the transcription factor linked sequentially from the N-terminus to the C-terminus, in which the second split intein is a C-terminal split intein, and the second fragment is a C-terminal fragment of the transcription factor.

In some embodiments, from the N-terminus to the C-terminus, the first fusion protein comprises a target protein linked to a first split intein linked to a first fragment of a transcription factor in which the first fragment is a C-terminal fragment of the transcription factor and the first split intein is a C-terminal split intein; and from the N-terminus to the C-terminus, the second fusion protein comprises a second fragment of the transcription factor linked to a second split intein linked to a candidate peptide, in which the second split intein is a N-terminal split intein and the second fragment is a N-terminal fragment of the transcription factor.

The first and second fusion proteins of the split intein-based selection system described herein may be used together with a nucleic acid comprising an inducible promoter operably linked to at least one reporter gene. Without being bound by a particular theory, binding of the (i) target protein in the first fusion protein with (ii) the candidate peptide in the second fusion protein brings the complementary split-intein in each fusion protein together to allow for protein splicing and release of a full-length transcription factor. The full-length transcription factor may then drive transcription from its cognate promoter. As used herein, a transcription factor is a protein that controls transcription (e.g., drives expression of a nucleic acid that is operably linked to a promoter). In some embodiments, a transcription factor binds to a promoter and drives transcription from the promoter. In some embodiments a transcription factor is an initiation factor. In some embodiments, a transcription factor is a sigma factor.

The promoter is operably linked to a reporter gene. A promoter is a control region of a nucleic acid sequence at which initiation and rate of transcription of the remainder of a nucleic acid sequence are controlled. A promoter may also contain sub-regions at which regulatory proteins and molecules may bind, such as RNA polymerase and other transcription factors. A promoter drives expression or drives transcription of the nucleic acid sequence that it regulates. A promoter is considered to be ‘operably linked’ to a nucleotide sequence when it is in a correct functional location and orientation in relation to the nucleotide sequence to control (‘drive’) transcriptional initiation and/or expression of that sequence. Promoters may be constitutive or inducible.

An inducible promoter is a promoter that is regulated (e.g., activated or inactivated) by the presence or absence of a particular factor. Inducible promoters for use in accordance with the present disclosure include any inducible promoter described herein or known to one of ordinary skill in the art. Examples of inducible promoters include, without limitation, chemically/biochemically-regulated and physically-regulated promoters such as alcohol-regulated promoters, tetracycline-regulated promoters (e.g., anhydrotetracycline (aTc)-responsive promoters and other tetracycline responsive promoter systems, which include a tetracycline repressor protein, steroid-regulated promoters (e.g., promoters based on the rat glucocorticoid receptor, human estrogen receptor, moth ecdysone receptors, and promoters from the steroid/retinoid/thyroid 25 receptor superfamily), metal-regulated promoters (e.g., promoters derived from metallothionein (proteins that bind and sequester metal ions) genes from yeast, mouse and human), pathogenesis-regulated promoters (e.g., induced by salicylic acid, ethylene or benzothiadiazole (BTH)), temperature/heat-inducible promoters (e.g., heat shock promoters), pH-regulated promoters, and light-regulated promoters. A non-limiting example of an inducible system that uses a light-regulated promoter is provided in Wang et al., Nat. Methods. 2012 Feb. 12; 9(3):266-9.

Non-limiting examples of inducible promoters include the inducible T5 lacO promoter, which may be induced by Isopropyl β-d-1-thiogalactopyranoside (IPTG), pCym promoter, which may be induced by cumate and a sigma-factor sensitive promoter, including an extra-cytoplasmic function (ECF) promoter.

In some embodiments, the promoter operably linked to a reporter gene is an extra-cytoplasmic function (ECF) promoter and the transcription factor is a sigma factor. In some embodiments, a Sigma factor comprises the N-terminal sequence ECF20_992 N (SEQ ID NO: 70) and the C-terminal sequence ECF20_992 C (SEQ ID NO: 69). Initiation of transcription in bacteria requires a sigma factor (a factor or specificity factor). Sigma factors bind to bacterial RNA polymerase to form a holoenzyme and initiate transcription. Non-limiting examples of sigma factors include extracytoplasmic function (ECF) a factors, a70 (RpoD), a19 (FecI), a24 (RpoE), a28 (RpoF/FliA), a32 (RpoH), a38 (RpoS), and 654 (RpoN). In some embodiments, a sigma factor is not a housekeeping sigma factor. In some embodiments, a sigma factor that is used is not native to a host cell and allows for orthogonal gene expression. As a non-limiting example, a sigma factor from B. subtilis that is not naturally expressed in E. coli may be used in E. coli for orthogonal gene expression. See also, e.g., Bervoets et al., Nucleic Acids Res. 2018 Feb. 28; 46(4): 2133-2144 and Pinto et al., Nucleic Acids Res. 2018 Aug. 21; 46(14):7450-7464. As would be appreciated by one of ordinary skill in the art, a particular sigma factor may require particular promoter elements to promote transcription and/or a particular environmental trigger including, e.g., heat. In some embodiments, additional activator proteins may be required for a sigma factor to function.

Non-limiting examples of reporter genes include genes that encode fluorescent proteins, enzymes, and antibiotic resistance genes. A reporter gene may allow for positive or negative selection.

In some embodiments, a reporter gene encodes a selection marker, such as an antibiotic resistance gene (e.g., bsd, neo, hygB, pac, ble, or Sh bla) and/or a gene encoding a fluorescent protein (RFP, BFP, YFP, or GFP). In some embodiments, the antibiotic resistance gene is cat, which encodes chloramphenicol acetyltransferase. Cells may be selected for resistance to chloramphenicol by culturing the cells in the presence of chloramphenicol. In some embodiments, the selection marker enables selection of cells expressing a protein of interest (e.g., a full-length transcription factor). As would be appreciated by one of ordinary skill in the art, the effective amount of a selection agent may vary depending on the host cell and phenotype of interest.

Positive selection markers are selection markers that confer a selective advantage to a host cell. In some embodiments, positive selection is the use of such selection markers to confer a growth or survival advantage to a cell comprising a protein of interest. In some embodiments, positive selection is used to identify cells in which a candidate peptide binds a target protein. Without being bound by a particular theory, protein splicing of the fusion proteins in the split intein-based selection system disclosed herein is dependent on the association of the candidate peptide with the target protein; therefore, in the absence of a binding interaction or when the binding interaction is weak, expression of the reporter gene is low. In some embodiments, a candidate peptide binder of a target protein increases expression of the reporter gene in a host cell comprising the split intein-based selection system disclosed herein by at least 10%, at least 20%, at least 30%, at least 40%, at 50%, at least 60%, at least 70%, at least 80%, at least 90%, at least 110%, at least 120%, at least 130%, at least 140%, at least 150%, at least 200%, at least 300%, at least 400%, at least 500%, at least 600%, at least 700%, at least 800%, at least 900%, or at least 100% relative to a control. In some embodiments, a control is a control peptide that has non-specific binding to the same target protein of interest. In some embodiments, a control is the level of expression of the candidate peptide binder in a host cell that comprises a split intein-based selection system with a control target protein that is not of interest.

Negative selection markers are selection markers that confer a selective disadvantage to a host cell. In some embodiments, negative selection is the use of such selection markers to confer a growth or survival disadvantage to a cell comprising an undesirable phenotype. Non-limiting examples of negative selection markers include Herpes Simplex Virus-1 Thymidine Kinase (HsvTK). Cells expressing HsvTK can be selected against by contacting cells with nucleotide 6-(β-D-2-deoxyribofuranosyl)-3,4-dihydro8H-pyrimido [4,5-c][1,2] oxazin-7-one (dP). Without being bound by a particular theory, expression of HsvTK alone without the addition of dP does not confer a growth disadvantage, which allows for temporal control of selection. As a non-limiting example, negative selection may be used to deplete host cells comprising candidate peptides that bind off-target proteins (identify candidates that non-specifically bind to a target protein of interest); the reporter gene may comprise a negative selection gene. For example, the split intein-based selection system described herein may be used with the candidate peptide and an off-target control protein in place of the target protein of interest to identify candidate peptides that bind to the off-target protein. In this embodiment, the inducible promoter may be operably linked to a gene encoding a negative selection marker and cells expressing the negative selection marker may be depleted by contacting the cells with the negative selection agent. Without being bound by a particular theory, the expression of the negative selection marker in this system is indicative of binding between the candidate peptide and the off-target control protein. In some embodiments, a reporter gene in the split intein-based selection system described herein comprises a negative selection marker to deplete cells that comprise an undesirable candidate peptide. As a non-limiting example, it may be desirable to select for peptide binders that specifically bind a target protein when the peptide is modified (e.g., comprising one or more post-translational modifications) but not when the peptide is unmodified. In some embodiments, the unmodified peptide is used in place of the candidate peptide in the split intein-based selection system described herein along with an inducible promoter operably linked to a negative selection marker and driving expression of the negative selection marker. The cells may be contacted with the negative selection agent to deplete cells with an unmodified peptide that binds to the target protein of interest. Without being bound by a particular theory, formation of a full-length transcription factor and subsequent expression of the full-length transcription factor would be dependent on the unmodified peptide binding to the target peptide in this system.

Expression of a reporter gene may be detected by any suitable method known in the art, including by analysis of RNA (e.g., reverse transcription-polymerase chain reaction (RT-PCR)), by analysis of protein levels (e.g., immunoassays), by analysis of enzyme activity (e.g., analysis of catalytic activity), by contacting cells with one or more selection agents, or by fluorescence analysis. A reporter protein may be detected by any known method, including via fluorescence microscopy, an immunoassay (including a western blot or an ELISA), or flow cytometry.

As one of ordinary skill in the art would appreciate, any transcriptional or translational output may be coupled with the first and second fusion proteins described herein.

In some embodiments, the intein-based selection system comprises a fusion protein with (i) a first fragment of a reporter protein, (ii) a first split intein, and (iii) a target protein, and another fusion protein that comprises (i) a candidate peptide, (ii) a second split intein, and (iii) a second fragment of the reporter protein. The first and second split inteins are complementary fragments, such that association of the first split intein with the second split intein promotes trans-splicing. In this embodiment, the presence of a full-length reporter protein is indicative of the candidate protein binding the target protein.

### Peptides

Ribosomally synthesized and post-translationally modified peptides (RiPPs) are a class of natural products that are modular and engineerable. In RiPP biosynthesis, the ribosome synthesizes a peptide using proteinogenic (i.e., amino acids that are biologically incorporated into proteins during translation) amino acids, and modifying enzymes subsequently bind to the peptide and modify it. Such post-translational modification introduces chemical diversity beyond the 20 standard amino acids, as well as structural diversity such as macrocyclization. Each modifying enzyme is constrained by a set of design rules, such as which amino acid(s) they can modify, the recognition site(s) (RSs) they will associate with, the distance (e.g., number of amino acids) between the RS and the amino acid residue(s) to be modified, and the amino acid context in which they can act (e.g., the amino acids in proximity to the target amino acid(s) that they modify). Synthetic peptides with particular activity (e.g., desired biological activity), and libraries thereof, can be constructed by incorporating the design constraints of one or a combination of modification enzymes into a peptide synthesis scheme.

In some embodiments, a RiPP comprises a leader amino acid sequence and a core amino acid sequence. In some embodiments, the leader and the core are connected via a cleavable linker (e.g., a protease-cleavable linker). In some embodiments, a RiPP comprises one or more (e.g., 1, 2, 3, 4, 5, 6, or more) recognition sites (RSs) for one or more distinct modification enzymes.

Aspects of the present disclosure relate to peptides for identification of binders to a target protein (e.g., candidate peptides or a plurality thereof) and peptides that may be useful in clinical applications. A candidate peptide is a peptide whose binding activity to a protein is being investigated. In some embodiments, a peptide comprises a sequence that is at least 60%, at least 70%, at least 80%, at least 90%, at least 91%, at least 92%, at least 93%, at least 94%, at least 95%, at least 96%, at least 97%, at least 98%, or at least 99% identical to SEQ ID NOs: 6-25 or 26-45, an amino acid sequence in Table 3 or any amino acid sequence disclosed herein, including fragments thereof.

The peptides described herein may be modified (e.g., the peptide may comprise a non-natural amino acid, a non-naturally occurring linkage, and/or a post-translational modification). In some embodiments, a modified peptide comprises a post-translational modification. In some embodiments, a modified peptide is produced recombinantly. In some embodiments, a modified peptide is produced synthetically. Without being bound by a particular theory, recombinant production of a modified peptide using a host cell may require expression of one or more protein modification enzymes. In some embodiments, the peptide is non-naturally occurring. In some embodiments, the peptide is naturally occurring.

Without being bound by a particular theory, a peptide comprising one or more modifications may be more stable (e.g., has reduced denaturation at a particular temperature), have increased bioavailability, and/or have increased solubility compared to a peptide not comprising the one or more modifications.

Non-limiting examples of post-translational modifications include formation of thioether bridges, formation of ester bridges, phosphorylation, glycosylation, acetylation, ubiquitylation/sumoylation, methylation, palmitoylation, myristoylation, prenylation, hydroxylation, GPI anchoring, ADP-ribosylation, pyrrolidone carboxylic acid, citrullination, S-nitrosylation, sulfation, amidation, nitration, oxidation, gamma-carboxyglutamic acid, topaquinone, lysine topaquinone, phosphopantetheine, quinone, hypusine, iodination, bromination, cysteine tryptophylquinone, formylation, and tryptophan tryptophylquinone.

In some embodiments, a peptide described herein is a ribosomally synthesized and post-translationally modified peptide (RiPP). RiPPs are ribosomally-produced peptides that comprise a post-translational modification. There are several subfamilies of RiPPs and RiPPs are grouped based on the biosynthetic machinery that produce the RiPP and structural characteristics. See, e.g., Table 1 below, which is based on Table 1 from Ortega and van der Donk, Cell Chem Biol. 2016 Jan. 21; 23(1):31-44; and Arnison et al., Nat Prod Rep. 2013 January;30(1):108-60.

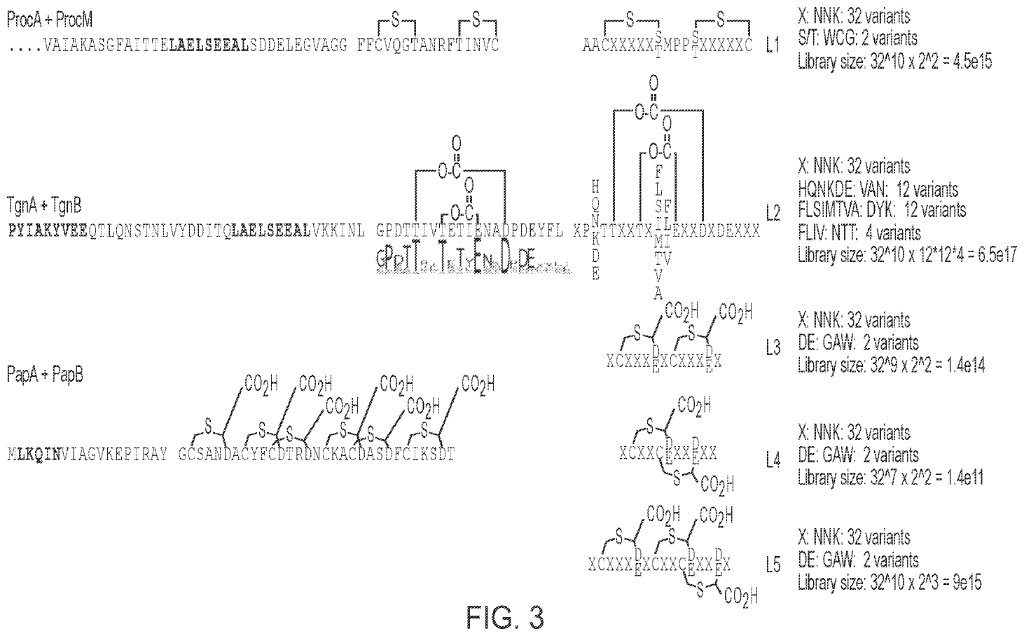

In some embodiments, a modified peptide comprises two or more (e.g., at least 3, at least 4, at least 5, at least 6, at least 7, at least 8, at least 9, at least 10, at least 11, at least 12, at least 13, at least 14, at least 15, at least 16, at least 17, at least 18, at least 19, at least 20) non-contiguous amino acids that are linked. In some embodiments, a modified peptide comprises at least 1 pair, at least 2 pairs, at least 3 pairs, at least 4 pairs, at least 5 pairs, at least 6 pairs, at least 7 pairs, a least 8 pairs, at least 9 pairs, at least 10 pairs, at least 15 pairs, at least 20 pairs, at least 30 pairs, at least 40 pairs, or at least 50 pairs) of non-contiguous amino acids that are linked. As a non-limiting example, scaffold L1 in FIG. 3 comprises two pairs of non-contiguous amino acids that are linked. As will be understood by one of ordinary skill in the art, two or more amino acids may be linked as valency permits.

In some embodiments, a peptide comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, a least 24, at least 25, at least 30, at least 40, at least 50, at least 60, at least 70, at least 80, at least 90, or at least 100 thioether bridges. In some embodiments, a peptide comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, a least 24, at least 25, at least 30, at least 40, at least 50, at least 60, at least 70, at least 80, at least 90, or at least 100 ester bridges. In some embodiments, a peptide comprises a thioether bridge and an ester bridge.

As an example, lanthipeptides comprise Lan and/or MeLan thioether bis-amino acids. In some embodiments, a peptide is a lanthipeptide. In some embodiments, a lanthipeptide comprises scaffold Li: AACX₁X₂X₃X₄X₅X₆MPPX₇X₈X₉X₁₀X₁₁X₁₂C (SEQ ID NO: 1), wherein: X₆ and X₇ are each the amino acid S or T; X₁-X₅ and X₈-X₁₂ are each any amino acid; and the peptide comprises a thioether bridge that links C at position 3 to S or T at position 9 in SEQ ID NO: 1 and a thioether bridge that links S or T at position 13 to C at position 19 in SEQ ID NO: 1. See, e.g., L1 in FIG. 3.

In some embodiments, a peptide is a microviridin. Microviridins may comprise lactones made from Glu/Asp and Ser/Thr side chains and/or lactams made from Lys and Glu/Asp residues. In some embodiments, a microviridin comprises X₁PX₂TTX₃X₄TX₅X₆X₇EX₈X₉DX₁₀DEX₁₁X₁₂X₁₃ (SEQ ID NO: 2) (scaffold L2), wherein: X₂ is the amino acid H, Q, N, K, D, or E; X₆ is the amino acid F, L, S, I, M, T, V, or A; X₇ is the amino acid F, L, I, or V; X₁, X₃-X₅ and X₈-X₁₃ are each any amino acid; and the peptide comprises an ester bridge that links T at position 5 of SEQ ID NO: 2 to D at position 15 of SEQ ID NO: 2 and an ester bridge that links T at position 8 of SEQ ID NO: 2 to E at position 12 of SEQ ID NO: 2. See, e.g., L2 in FIG. 3.

In some embodiments, a peptide comprises a sactipeptide (ranthipeptide). Sactipeptides comprise one or more intramolecular thioether linkages between Cys side chains and α-carbons of other amino acids. In some embodiments, a sactipeptide comprises: X₁CX₂X₃X₄X₅X₆CX₇X₈X₉X₁₀X₁₁ (SEQ ID NO: 3) (scaffold L3), wherein: X₅ and X₁₀ are each the amino acid D or E; X₁-X₄, X₆-X₉, and X₁₁ are each any amino acid; and the peptide comprises a thioether bridge that links C at position 2 to D or E at position 6 of SEQ ID NO: 3 and a thioether bridge that links C at position 8 to D or E at position 12 of SEQ ID NO: 3. See, e.g., L3 in FIG. 3. In some embodiments, a peptide comprising scaffold L3 comprises a sequence that is at least 60%, at least 70%, at least 80%, at least 90%, at least 91%, at least 92%, at least 93%, at least 94%, at least 95%, at least 96%, at least 97%, at least 98%, or at least 99% identical to a sequence selected from SEQ ID NOs: 17-25.

In some embodiments, a sactipeptide comprises X₁CX₂X₃CX₄X₅X₆X₇X₈X₉ (SEQ ID NO: 4) (scaffold L4), wherein: X₄ and X₇ are each the amino acid D or E; X₁-X₃, X₅-X₆, and X₈-X₉ are each any amino acid; and the peptide comprises a thioether bridge that links C at position 2 to D or E at position 6 of SEQ ID NO: 4 and a thioether bridge that links C at position 5 to D or E at position 9 of SEQ ID NO: 4. See, e.g., L4 in FIG. 3. In some embodiments, a sactipeptide comprises X₁CX₂X₃X₄X₅X₆CX₇X₈CX₉X₁₀X₁₁X₁₂X₁₃ (SEQ ID NO: 5), wherein: X₅, X₉, and X₁₂ are each the amino acid D or E; X₁-X₄, X₆-X₈, X₁₀-X₁₁, and X₁₃ are each any amino acid; and the peptide comprises a thioether bridge that links the C at position 2 to D or E at position 6 of SEQ ID NO: 5, a thioether bridge that links C at position 8 of SEQ ID NO: 5 with D or E at position 12 of SEQ ID NO: 5, and a thioether bridge that links C at position 11 with D or E at position 15 of SEQ ID NO: 5. See, e.g., L5 in FIG. 3. In some embodiments, a peptide comprising scaffold L5 comprises a sequence that is at least 60%, at least 70%, at least 80%, at least 90%, at least 91%, at least 92%, at least 93%, at least 94%, at least 95%, at least 96%, at least 97%, at least 98%, or at least 99% identical to a sequence selected from SEQ ID NOS: 6-16.

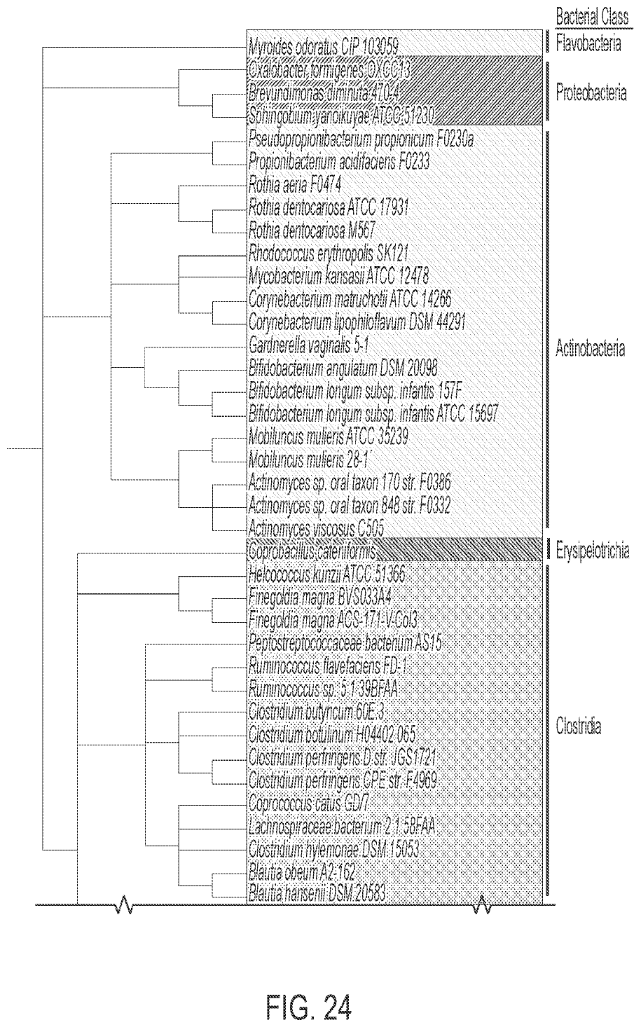

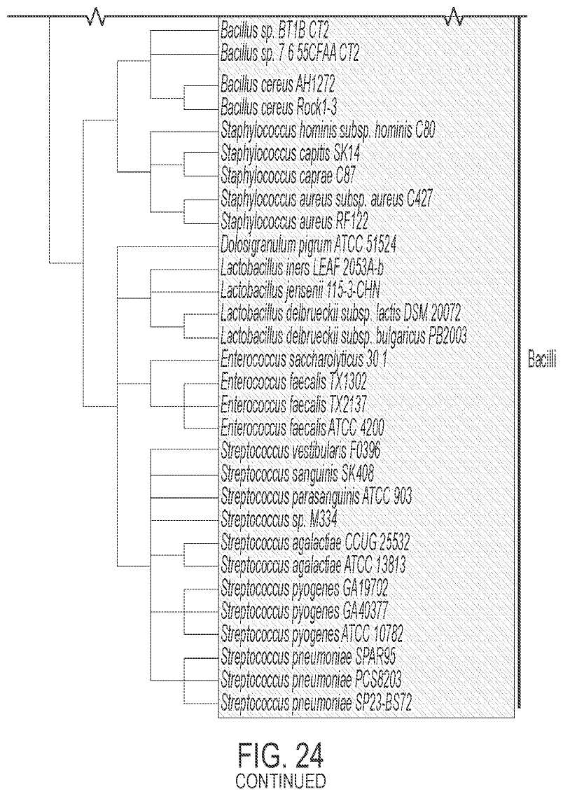

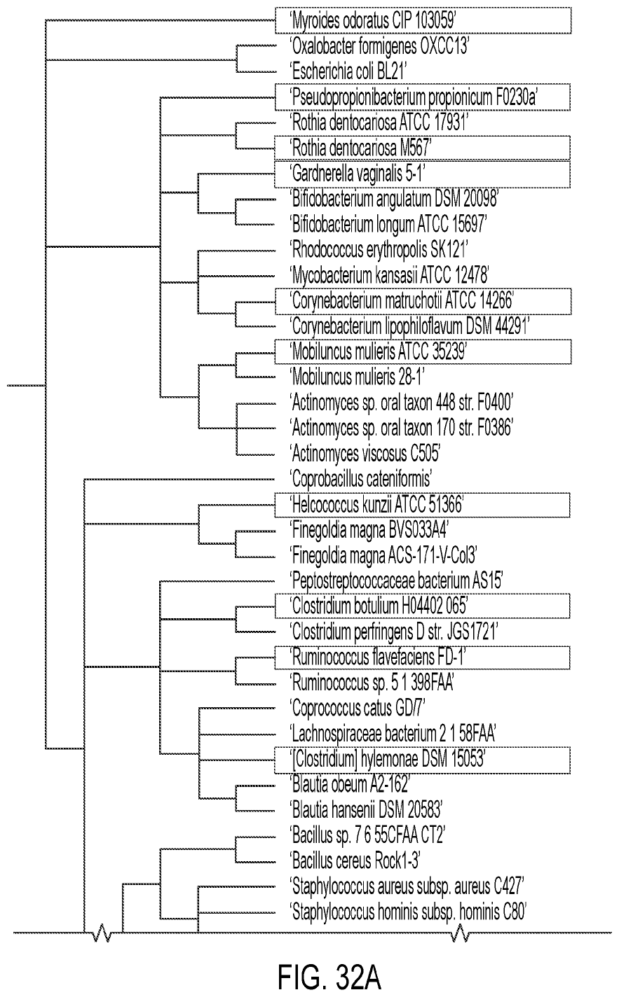

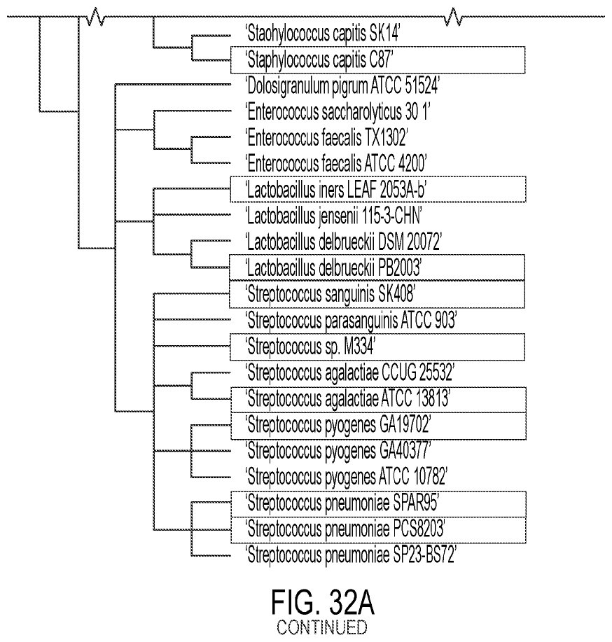

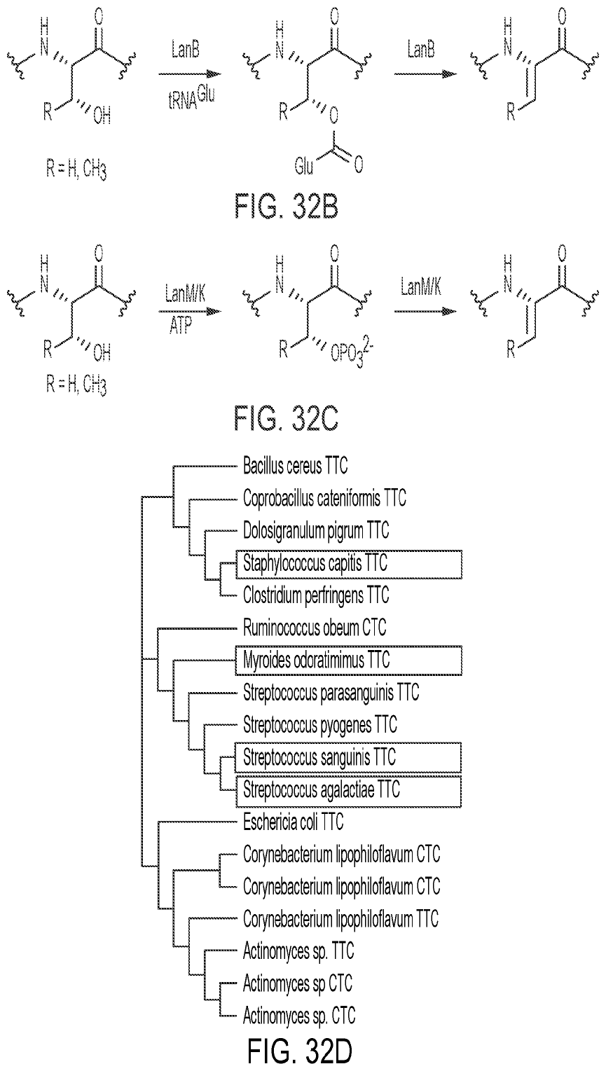

In some embodiments, a peptide described herein has biological activity, e.g., antimicrobial activity. In some embodiments, peptides having antimicrobial activity are modified from RiPPs of microbiome bacteria from a subject, such as a human subject. Non-limiting examples of bacteria from which RiPPs can be modified to have antimicrobial activity include the Flavobacteria, Proteobacteria, Actinobacteria, Erysipelotrichia, Clostridia, Bacilli provided in FIG. 24, or the bacteria provided in FIG. 32A and FIG. 32D. In some embodiments, a peptide having antimicrobial activity (e.g., a modified RiPP) comprises at least 15 (e.g., 15, 16, 17, 18, 19, 20, 21, 22, 23, 24, 25, 26, 27, 28, 29, 30, 31, 32, 33, 34, 35, 36, 37, 39, 39, 40, 41, or 42) consecutive amino acids of the sequence GTFSX₁GX₂X₃X₄X₅X₆X₇X₈X₉X₁₀X₁₁GX₁₂DGVX₁₃X₁₄TX₁₅SHECHMNTWQFLX₁₆TCCS (SEQ ID NO: 88) or GX₁₂DGVX₁₃X₁₄TX₁₅SHECHMNTWQFL (SEQ ID NO: 938); wherein: X₁ is G or E; X₂ is W or T; X₃ is F or I; X₄ is T or S; X₅ is A or I; X₆ is I or T; X₇ is Q or L; X₈ is L or S; X₉ is T, V, or G; X₁₀ is L, Y, or S; X₁₁ is A, M, R, or G; X₁₂ is R, G, N, W, or K; X₁₃ is W, M, V, L or F; X₁₄ is F, H, C, P, or K; X₁₅ is G, L, W, V, or I; and X₁₆ is L, F, or A. In some embodiments, a peptide having antimicrobial activity (e.g., a modified RiPP) comprises a sequence that is at least 60%, at least 70%, at least 80%, at least 90%, at least 91%, at least 92%, at least 93%, at least 94%, at least 95%, at least 96%, at least 97%, at least 98%, or at least 99% identical to a sequence selected from GGWFTAIQLTLAGRCGNWFTGSFECTSNNVKCG (SEQ ID NO: 89); GGDGVMHTLTHECHMNTWQFLLTCC (SEQ ID NO: 90); GTFSEGTISITLSVYMGNDGKVCTWTVECQNNCSHKK (SEQ ID NO: 91); GSRWWQGVLPTVSHECRMNSFQHIFTCC (SEQ ID NO: 92); or GGKNGVFKTISHECHLNTWAFLATCCS (SEQ ID NO: 93). In some embodiments, a peptide having antimicrobial activity (e.g., a modified RiPP) comprises a sequence selected from GGWFTAIQLTLAGRCGNWFTGSFECTSNNVKCG (SEQ ID NO: 88); GGDGVMHTLTHECHMNTWQFLLTCC (SEQ ID NO: 90); GTFSEGTISITLSVYMGNDGKVCTWTVECQNNCSHKK (SEQ ID NO: 91); GSRWWQGVLPTVSHECRMNSFQHIFTCC (SEQ ID NO: 92); and GGKNGVFKTISHECHLNTWAFLATCCS (SEQ ID NO: 93). In some embodiments, a peptide having antimicrobial activity (e.g., a modified RiPP) comprises a sequence that is at least 60%, at least 70%, at least 80%, at least 90%, at least 91%, at least 92%, at least 93%, at least 94%, at least 95%, at least 96%, at least 97%, at least 98%, or at least 99% identical to a sequence selected from IDTLDYEISHQELSGKSAAGWQTAFRLTMQGRCGGVFTLSYECATPHVSCG (SEQ ID NO: 97); GGWYTAFKLTLAGRCGLCFTCSYECTSNNVHC (SEQ ID NO: 98); and GWFTAIQLTLAGRCGNWFTGSFECTSNNVKCG (SEQ ID NO: 99). In some embodiments, a peptide having antimicrobial activity (e.g., a modified RiPP) comprises a sequence selected from IDTLDYEISHQELSGKSAAGWQTAFRLTMQGRCGGVFTLSYECATPHVSCG (SEQ ID NO: 97); GGWYTAFKLTLAGRCGLCFTCSYECTSNNVHC (SEQ ID NO: 98); and GWFTAIQLTLAGRCGNWFTGSFECTSNNVKCG (SEQ ID NO: 99). In some embodiments, a peptide having antimicrobial activity (e.g., a modified RiPP) comprises a sequence that is at least 60%, at least 70%, at least 80%, at least 90%, at least 91%, at least 92%, at least 93%, at least 94%, at least 95%, at least 96%, at least 97%, at least 98%, or at least 99% identical to a sequence selected from SEQ ID NOs: 115-147, 758-783, 820, and 821. In some embodiments, a peptide having antimicrobial activity (e.g., a modified RiPP) comprises a sequence of any one of SEQ ID NOs: 115-147, 758-783, 820, and 821. In some embodiments, a peptide having antimicrobial activity comprises at least 15 (e.g., 16, 17, 18, 19, 20, 21, 22, 23, 24, 25, 26, 27, 28, 29, 30, or more) consecutive amino acids of any one of Lacticin 481, AMK287, AMK417, AMK419, AMK687, or AMK691, or of of any one of SEQ ID NOs: 115-147, 758-783, 820, or 821. In some embodiments, a peptide having antimicrobial activity (e.g., a modified RiPP) comprises at least 15 (e.g., 15, 16, 17, 18, 19, 20, 21, 22, 23, 24, 25, 26, 27, 28, 29, 30, 31, 32, 33, or 34) consecutive amino acids of the sequence GGGWX₁TAFX₂LTLAGRCGX₃X₄FTX₅SYECTSNNVX₆CG (SEQ ID NO: 94), wherein: X₁ is F, Y, or Q; X₂ is Q, K, or R; X₃ is N, L, or G; X₄ is W, C, or V; X₅ is G, C, or L; X₆ is K, H, or S. In some embodiments, a peptide having antimicrobial activity (e.g., a modified RiPP) comprises a sequence that is at least 60%, at least 70%, at least 80%, at least 90%, at least 91%, at least 92%, at least 93%, at least 94%, at least 95%, at least 96%, at least 97%, at least 98%, or at least 99% identical to a sequence selected from GGGWFTAIQLTLAGRCGNWFTGSFECTSNNVKCG (SEQ ID NO: 100), GGGWYTAFKLTLAGRCCGLCFTCSYECTSNNVHC (SEQ ID NO: 101), and GWQTAFRLTMQGRCGGVFTLSYECATPHVSCG (SEQ ID NO: 96). In some embodiments, a peptide having antimicrobial activity (e.g., a modified RiPP) comprises a sequence selected from GGGWFTAIQLTLAGRCGNWFTGSFECTSNNVKCG (SEQ ID NO: 100), GGGWYTAFKLTLAGRCCGLCFTCSYECTSNNVHC (SEQ ID NO: 110), and GWQTAFRLTMQGRCGGVFTLSYECATPHVSCG (SEQ ID NO: 96).

In some embodiments, a peptide having antimicrobial activity (e.g., a modified RiPP) comprises at least 15 (e.g., 15, 16, 17, 18, 19, 20, 21, 22, 23, 24, 25, 26, 27, 28, 29, 30, 31, 32, 33, or 34) consecutive amino acids of the sequence GSX₁GX₂X₃GVX₄X₅TX₆SHECHMNTWQFLX₇TCCS (SEQ ID NO: 95), wherein: X₁ is R or G; X₂ is G, W, or K; X₃ is D, Q, or N; X₄ is M, L, or F; X₅ is H, P, or K; X₆ is L, V, or I; and X₇ is L, F, or A;. In some embodiments, a peptide having antimicrobial activity (e.g., a modified RiPP) comprises a sequence that is at least 60%, at least 70%, at least 80%, at least 90%, at least 91%, at least 92%, at least 93%, at least 94%, at least 95%, at least 96%, at least 97%, at least 98%, or at least 99% identical to a sequence selected from GGDGVMHTLTHECHMNTWQFLLTCC (SEQ ID NO: 90), GSRWWQGVLPTVSHECRMNSFQHIFTCC (SEQ ID NO: 92), and GGKNGVFKTISHECHLNTWAFLATCCS (SEQ ID NO: 93). In some embodiments, a peptide having antimicrobial activity (e.g., a modified RiPP) comprises a sequence selected from GGDGVMHTLTHECHMNTWQFLLTCC (SEQ ID NO: 90), GSRWWQGVLPTVSHECRMNSFQHIFTCC (SEQ ID NO: 92), and GGKNGVFKTISHECHLNTWAFLATCCS (SEQ ID NO: 93).

In some embodiments, a peptide having antimicrobial activity (e.g., a modified RiPP) comprises at least 15 (e.g., 15, 16, 17, 18, 19, 20, 21, 22, 23, 24, 25, 26, 27, 28, 29, 30, 31, 32, 33, 34, 35, 36, 37, 38, 39, 40, 41, or 42) consecutive amino acids of the sequence GWX₁WGSYRDX₂YGALRGPNX₃X₄FVGX₅GGX₆X₇X₈X₉X₁₀X₁₁X₁₂X₁₃X₁₄SWRLVPR (SEQ ID NO: 102), wherein: X₁ is I, F, L, or Y; X₂ is V or I; X₃ is P, S, T, or K; X₄ is P, G, N, or R; X₅ is L, G, A, or R; X₆ is V, F, or S; X₇ is P, T, or S; X₈ is P, G, or E; X₉ is G or W; X₁₀ is G or R; X₁₁ is V or L; X₁₂ is S or V; X₁₃ is G or P; and X₁₄ is G or R. In some embodiments, a peptide having antimicrobial activity (e.g., a modified RiPP) comprises a sequence that is at least 60%, at least 70%, at least 80%, at least 90%, at least 91%, at least 92%, at least 93%, at least 94%, at least 95%, at least 96%, at least 97%, at least 98%, or at least 99% identical to a sequence selected from GLIYGKYRDVLSGARLVTPPEVALRLVPR (SEQ ID NO: 103), GWFWGSYRDIFGALRGPNSGFEGGGGFTGGGVSGGSWRLVPR (SEQ ID NO: 104), GWLWGSYRDVYGVWHGPRTNFNGAGGSSEWRLVPR (SEQ ID NO: 105), and GWYWGNRRDIYGALRYANKRLVPR (SEQ ID NO: 106). In some embodiments, a peptide having antimicrobial activity (e.g., a modified RiPP) comprises a sequence selected from GLIYGKYRDVLSGARLVTPPEVALRLVPR (SEQ ID NO: 103), GWFWGSYRDIFGALRGPNSGFEGGGGFTGGGVSGGSWRLVPR (SEQ ID NO: 104), GWLWGSYRDVYGVWHGPRTNFNGAGGSSEWRLVPR (SEQ ID NO: 105), and GWYWGNRRDIYGALRYANKRLVPR (SEQ ID NO: 106).

In some embodiments, a peptide having antimicrobial activity (e.g., a modified RiPP) comprises a sequence that is at least 60%, at least 70%, at least 80%, at least 90%, at least 91%, at least 92%, at least 93%, at least 94%, at least 95%, at least 96%, at least 97%, at least 98%, or at least 99% identical to GVGYbbYWGILPLVbKNPQIAPVaENbVKARLL (SEQ ID NO: 107), wherein ‘b’ is dehydrobutyrine and ‘a’ is dehydroalanine, and wherein a thioether bridge connects the dehydrobutyrine at position 15 to the alanine at position 21, and a thioether bridge connects the dehydrobutyrine at position 27 to the alanine at position 30. In some embodiments, a peptide having antimicrobial activity (e.g., a modified RiPP) comprises the sequence GVGYbbYWGILPLVbKNPQIAPVaENbVKARLL (SEQ ID NO: 107), wherein ‘b’ is dehydrobutyrine and ‘a’ is dehydroalanine, and wherein a thioether bridge connects the dehydrobutyrine at position 15 to the alanine at position 21, and a thioether bridge connects the dehydrobutyrine at position 27 to the alanine at position 30.

In some embodiments, a peptide having antimicrobial activity (e.g., a modified RiPP) comprises a sequence that is at least 60%, at least 70%, at least 80%, at least 90%, at least 91%, at least 92%, at least 93%, at least 94%, at least 95%, at least 96%, at least 97%, at least 98%, or at least 99% identical to a sequence provided in Table 12 (SEQ ID NOs: 115-147).

In some embodiments, a peptide having antimicrobial activity is selectively active against a particular class, genera, species, or strain of bacteria. In some embodiments, a peptide having antimicrobial activity does not kill commensal bacteria of a subject. In some embodiments, a peptide having antimicrobial activity kills pathogenic bacteria. In some embodiments, a peptide having antimicrobial activity is selective towards pathogenic bacteria over commensal bacteria. In some embodiments, a peptide having antimicrobial activity is selective towards bacteria of a first class, genera, species, or strain over bacteria of a second class, genera, species or strain. In some embodiments, being selective towards a first population of bacteria over a second population of bacteria means the peptide kills bacteria of the first population of bacteria at a concentration that is at least 5% lower (e.g., at least 10% lower, 15% lower, 20% lower, 25% lower, 30% lower, 35% lower, 40% lower, 45% lower, 50% lower, 55% lower, 60% lower, 65% lower, 70% lower, 75% lower 80% lower, 85% lower, 86% lower, 87% lower, 88% lower, 89% lower, 90% lower, 91% lower, 92% lower, 93% lower, 94% lower, 95% lower, 96% lower, 97% lower, 98% lower, or 99% lower) than the concentration that is required to kill bacteria of the second population. In some embodiments, being selective towards a first population of bacteria over a second population of bacteria means the peptide is capable of killing bacteria of the first population, but is unable to kill bacteria of the second population.

In some embodiments, a peptide having antimicrobial activity (e.g., a modified RiPP) disclosed herein comprises one or more post-translational modifications, such as modifications effected by one or more enzymes listed in Tables 5, 7, 8, 13, 14, and 17. Possible peptide post-translational modifications include, but are not limited to, phosphorylation (e.g., of serine, threonine, or tyrosine residues); glycosylation (e.g., N-glycosylation, O-glycosylation, glypiation, C-glycosylation, and phosphoglycosylation); ubiquitylation/ubiquitination; S-nitrosylation; methylation (e.g., N-methylation or O-methylation); N-acetylation; lipidation (e.g., C-terminal glycosyl phosphatidylinositol (GPI) anchor, N-terminal myristoylation, S-myristoylation, or S-prenylation); deamidation; eliminylation; prenylation; ADP-ribosylation; hydroxylation; polypeptide backbone modifications (e.g., stereoisomerization, dehydration, oxidation, cyclization), and any other post-translational modifications disclosed herein. Post-translational modifications are described further in Müller Biochemistry 2018, 57(2):177-187 (doi: 10.1021/acs.biochem.7b00861) and deGruyter et al. Biochemistry 2017, 56(30):3863-3873 (doi: 10.1021/acs.biochem.7b00536).

In some embodiments, one or more serine (S) and/or cysteine (C) residues of a peptide having antimicrobial activity disclosed herein is replaced with a dehydroalanine (e.g., by dehydration of a serine or cysteine). In some embodiments, one or more threonine (T) residues of a peptide having antimicrobial activity disclosed herein is replaced with a dehydrobutyrine (e.g., by dehydration of a threonine). In some embodiments, a peptide having antimicrobial activity (e.g. a modified RiPP) disclosed herein comprises one or more thioether bridges, one or more thioester bridges, and/or one or more other bridges. Any modified peptide disclosed herein can comprise any combination of post-translational modifications described herein (e.g., one or more dehydrated amino acids, one or more thioether bridges, one or more thioester bridges, and/or one or more other bridges).

Despite the structural diversity of RiPPs, RiPP biosynthesis generally begins with production of a precursor peptide by ribosomes; the precursor peptide generally comprises an N-terminal leader sequence and a C-terminal core sequence that comprises sites for post-translational modification. In some embodiments, biosynthesis requires a C-terminal recognition sequence. The leader sequence recruits the biosynthetic machinery and is, in some embodiments, cleaved by a peptidase to form a mature peptide. In some embodiments, a protein modification enzyme is a peptidase that cleaves the leader peptide.

In some embodiments, one or more protein modification enzymes (e.g., at least 1, at least 2, at least 3, at least 4, at least 5, at least 6, at least 7, at least 8, at least 9, or at least 10) protein modification enzymes may be expressed in a cell to produce a modified peptide. In some embodiments, the protein modification enzyme is expressed from a heterologous nucleic acid. The expression of one or more protein modification enzymes may be under the control of an inducible promoter.

Protein modification enzymes including RiPP synthesis enzymes are known. As a non-limiting example, Prochlorosin (ProcM) is a member of the enzyme class that installs the macrocyclic thioether linkages that give rise to lanthipeptides. ProcM engages in dehydration-based chemistry that targets side chain serine/threonine residues. ProcA is a natural peptide substrate for ProcM. TgnB is a member of the enzyme class that installs the macrocyclic ester linkages that give rise to microviridins. TgnA is a natural peptide substrate for the modifying enzyme, TgnB. PapB is a member of the enzyme class that installs the macrocyclic thioether linkages that give rise to ranthipeptides, or sactipeptides. Freyrasin (PapB) engages in radical-based chemistry that targets main chain carbon atoms of aspartate/glutamate residues. LynD is a cyanobactin cyclodehydratase (PDB ID 4V1T). Additional non-limiting examples of protein modification enzymes including RiPP synthesis enzymes are provided in Table 7. See also, e.g., Ortega and van der Donk, Cell Chem Biol. 2016 Jan. 21; 23(1): 31-44. In some embodiments, a protein modification enzyme comprises a sequence that is at least 60%, at least 70%, at least 80%, at least 90%, at least 91%, at least 92%, at least 93%, at least 94%, at least 95%, at least 96%, at least 97%, at least 98%, or at least 99% identical to a sequence selected from SEQ ID NOs: 80-83, 174, 176, 179, 180, 183, 185, 187, 188, 190, 192, 247, 249-251, 253, 255, 256, 258, 262, 264, 265, 267, 270, 271, 274, 275, 279-281, 285, 289, 290, 292, 295, 296, 298, 300-303, 305, 308-310, 312, 313, 316, 318-322, 325, 327, 330, 332, 334, 335, 337, 338, 342, 343, 346, 349, 350, 354, 356, 360, 362, and 363. In some embodiments, a protein modification enzyme comprises a sequence selected from SEQ ID NOs: 80-83, 174, 176, 179, 180, 183, 185, 187, 188, 190, 192, 247, 249-251, 253, 255, 256, 258, 262, 264, 265, 267, 270, 271, 274, 275, 279-281, 285, 289, 290, 292, 295, 296, 298, 300-303, 305, 308-310, 312, 313, 316, 318-322, 325, 327, 330, 332, 334, 335, 337, 338, 342, 343, 346, 349, 350, 354, 356, 360, 362, and 363. See, e.g., Table 4, Table 7, Table 8, Table 9, and Table 17.

In some embodiments, the split intein-based selection methods described herein comprise sequencing to identify the candidate peptide in the host cell. In some embodiments, a host cell comprises a plasmid encoding the candidate peptide and the plasmid may be sequenced. Non-limiting examples of sequencing methods include next-generation sequence (NGS), nanopore sequencing, and Sanger sequencing.

TABLE 1
Non-limiting examples of RiPP modified peptides.

| RiPP Subfamily | Defining Features |
|---|---|
| Amatoxins and | N-to-C cyclized peptides produced by fungi |
| Phallotoxins | |
| Autoinducing | Peptides containing a cyclic ester or a thioester. |
| peptides | |
| Bacterial head-to- | N-to-C cyclized peptides differing from cyanobactins in the biosynthetic machinery employed |
| tail cyclized | for macrocyclization |
| peptides | |
| Bottromycins | An N-terminal macrocyclic amidine |
| | Use a C-terminal follower peptide instead of N-terminal leader peptide. |
| | Use a C-terminal follower peptide instead of N-terminal leader peptide. |
| Conopeptides | Venom peptides produced by snails. The degree and type of PTMs varies. |
| Cyanobactins | N-to-C macrocyclic peptides produced by cyanobacteria. |
| | Sometimes further decorated with azole(in)es and/or prenylations. |
| Cyclotides | N-to-C cyclized peptides produced by plants containing a cysteine knot composed of three |
| | disulfides |
| Glycocins | Glycosylated antimicrobial peptides |
| Lanthipeptides | Lan and/or MeLan thioether bis-amino acids |
| Lasso peptides | An N-terminal macrolactam with the C-terminal tail threaded through the ring. |
| Linaridins | Dehydroamino acids but lacking Lan/MeLan |
| Linear azol(in)e- | Linear peptides containing (methyl)oxazol(in)e or/and thiazol(in)e heterocycles |
| containing | |
| peptides | |
| Methanobactin | Peptidic chelators used by methanotrophic bacteria |
| Microcins | Produced by members of the Enterobacteriaceae Family. |
| | Include lasso peptide and LAP families |
| Microviridins | Lactones made from Glu/Asp and Ser/Thr side chains and/or lactams made from Lys and |
| | Glu/Asp residues |
| Orbitides | N-to-C cyclized peptides produced by plants lacking disulfides |
| Proteusins | Linear peptides containing D-amino acids and C-methylations |
| Pyrroloquinoline | Small molecules generated from the post-translational modification of a precursor peptide or |
| quinone (PQQ), | protein. |
| Pantocin, and | |
| Thyroid hormones | |
| Sactipeptides | Intramolecular thioether linkages between Cys side chains and α-carbons of other amino acids |
| (Ranthipeptides) | |
| Streptide | A Trp-to-Lys carbon-carbon cross link |
| Thioamides | Peptides containing thioamide linkages installed post-translationally |
| Thiopeptides | A central six-membered nitrogen-containing ring |
| | Additional PTMs include dehydrations and cyclodehydrations |

TABLE 7
Non-limiting examples of peptide modifying enzymes

| Protein | | | |
|---|---|---|---|
| modifi- | | | |
| cation | | | Peptide |
| enzyme | Enzyme | | interaction |
| name | class | Modification facilitated | mechanism |
| TgnB | lactone cyclase | 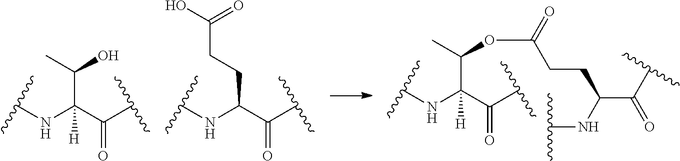 | Leader- dependent |
| PaaA | glu-glu cyclase | 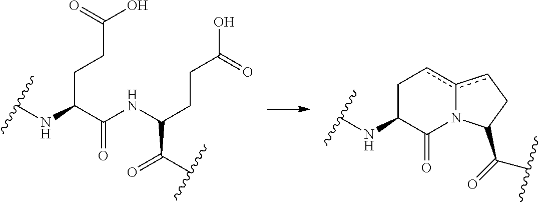 | Leader- dependent |
| PlpXY | tyrosine excisionase | 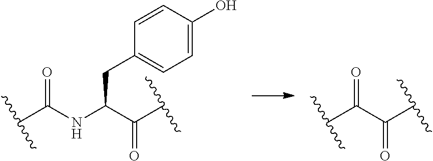 [SMILES: *C(=O)N[C@@H](Cc1ccc(O)cc1)C(*)=O ; *C(=O)C(*)=O] | Leader- dependent |
| LynD | thiazoline cyclase | 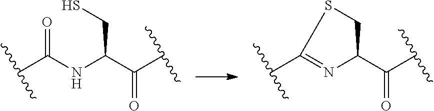 [SMILES: *C(=O)N[C@@H](CS)C(*)=O ; *C(=O)[C@@H]1CSC(*)=N1] | Leader- dependent |
| LasF | carboxylic acid methyl- transferase | 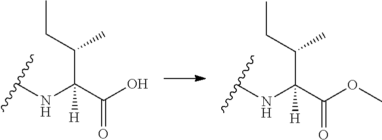 | Tailoring |
| PalS | cysteine glycosyl- transferase | 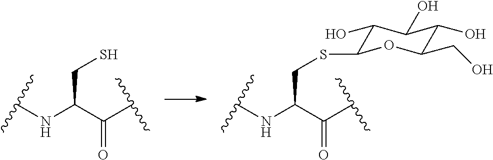 [SMILES: *N[C@@H](CS)C(*)=O ; *N[C@@H](CSC1OC(CO)C(O)C(O)C1O)C(*)=O] | Tailoring |
| EpiD | de- carboxylase | 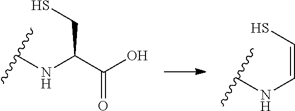 [SMILES: *N[C@@H](CS)C(=O)O ; *N/C=C\S] | Tailoring |
| ThcoK | serine kinase | 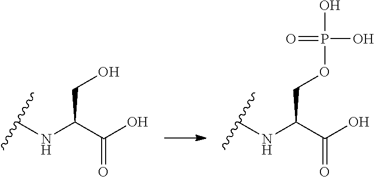 [SMILES: *N[C@@H](CO)C(=O)O ; *N[C@@H](COP(=O)(O)O)C(=O)O] | Tailoring |
| PadeK | serine kinase | 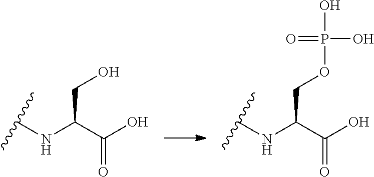 [SMILES: *N[C@@H](CO)C(=O)O ; *N[C@@H](COP(=O)(O)O)C(=O)O] | Tailoring |

### Methods of Engineering RiPPs and RiPP Libraries

Provided herein are methods for engineering RiPPs, such as to develop non-naturally occurring RiPPs with desired properties. Both the leader and core sequences of a RiPP can be engineered based on the methods provided. In a leader sequence, recognition site(s) (RS) for protein modifying enzymes can be engineered (e.g., added, removed, optimized, or moved), such as to enable the use of the corresponding protein modifying enzyme to incorporate a particular post-translational modification to a peptide, or to prevent a particular protein modifying enzyme from acting on a given RiPP. In a core sequence, the amino acid sequence can be engineered, such as to facilitate post-translational modification by a particular protein modifying enzyme.

The amino acid sequence of a RiPP (including its leader and core sequences, as well as any additional amino acids within the RiPP) determine which protein modifying enzymes interact with the RiPP. Leader-dependent protein modifying enzymes associate with an RS within the leader sequence of a RiPP, and facilitate modification of an amino acid or amino acids within the core sequence. Tailoring protein modification enzymes associate with a particular amino acid or amino acids within the core sequence of a RiPP, and facilitate modification of one or more of those amino acids.

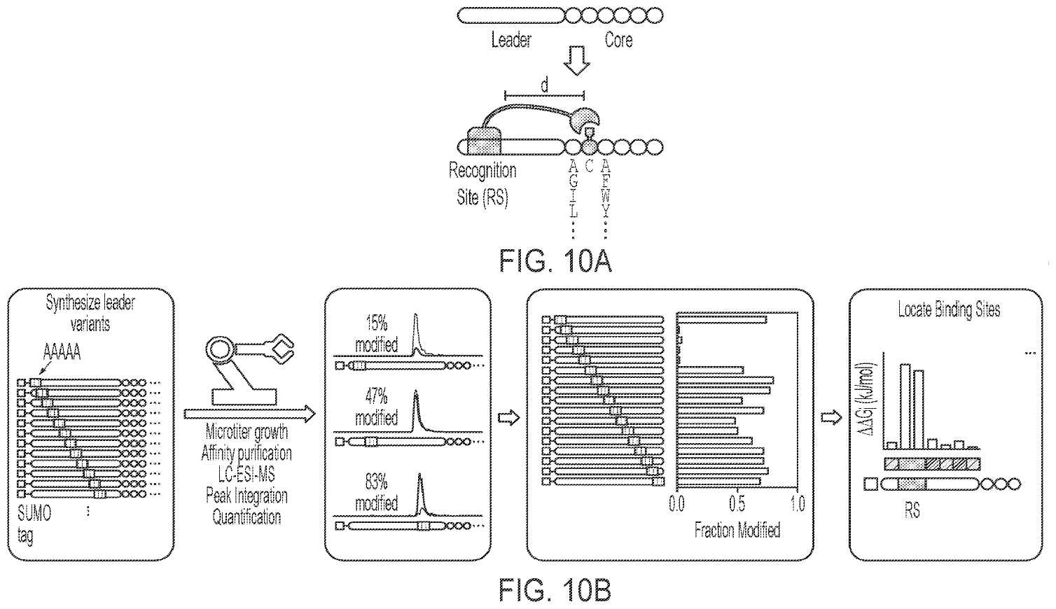

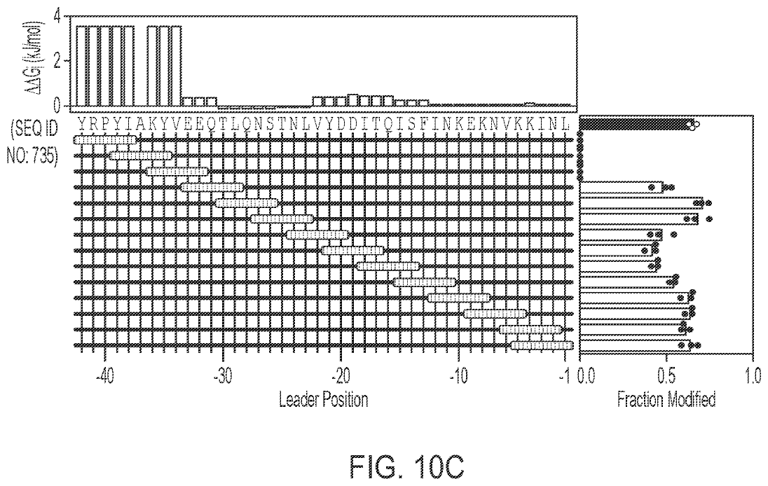

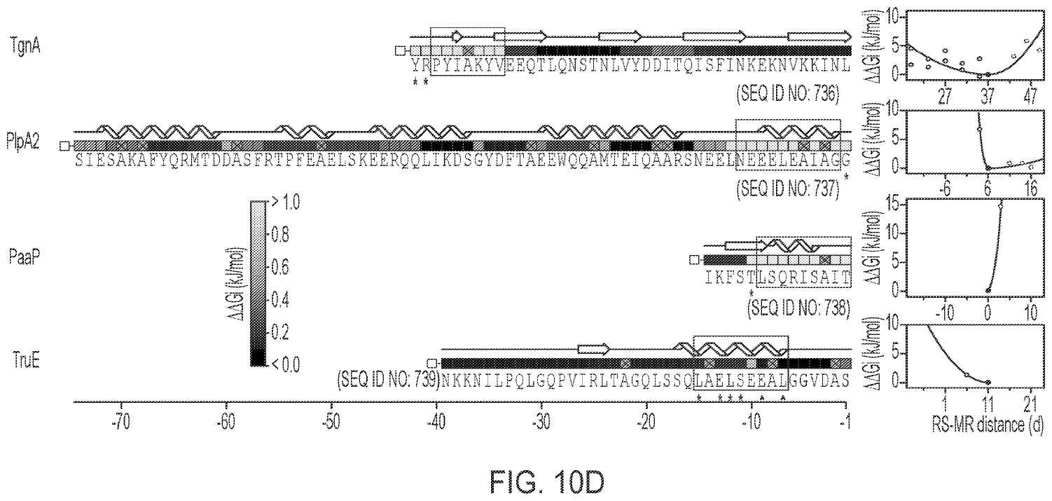

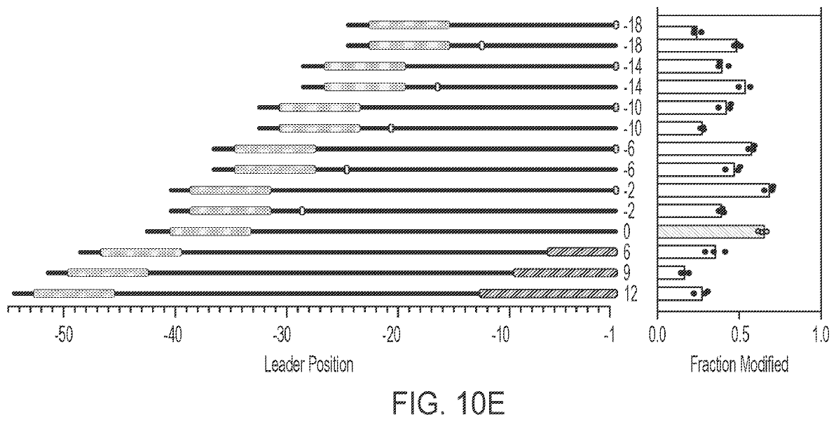

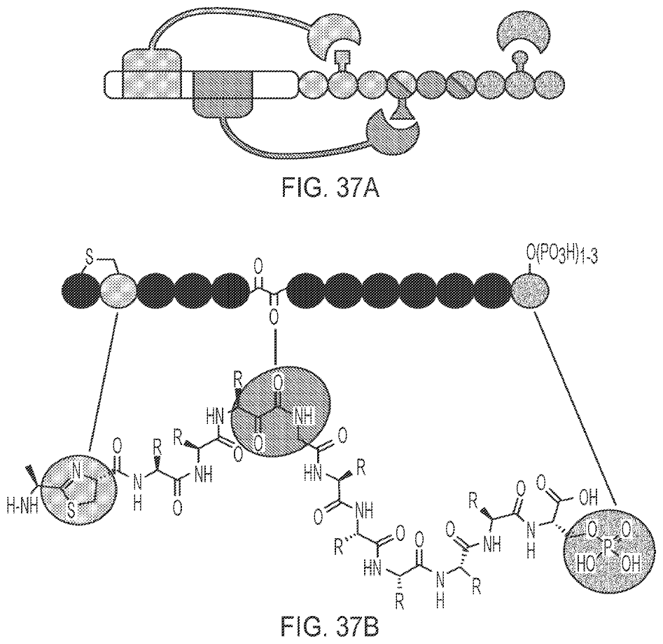

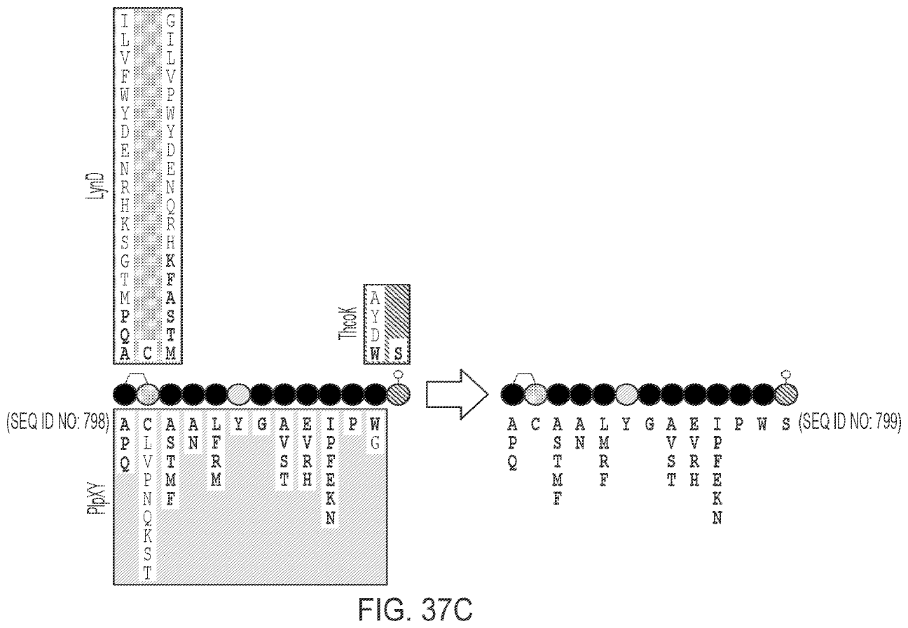

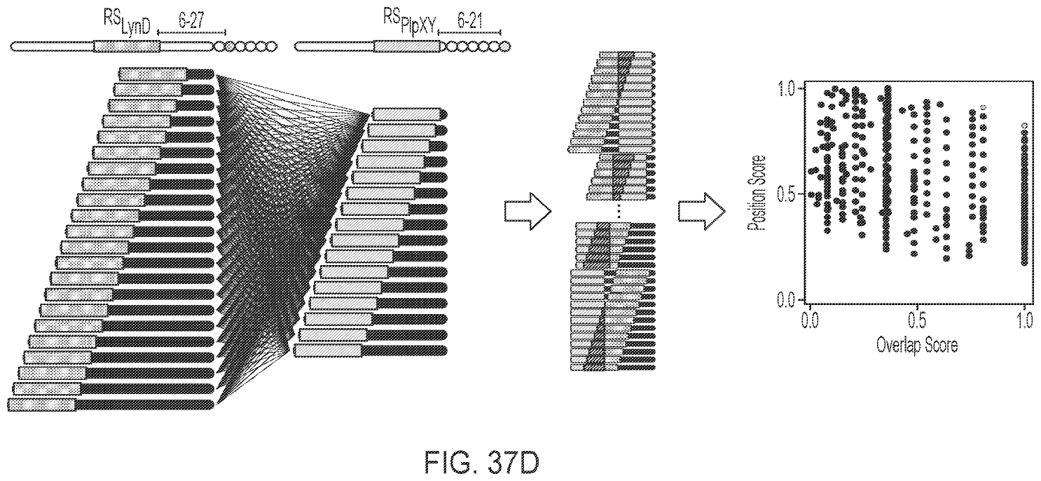

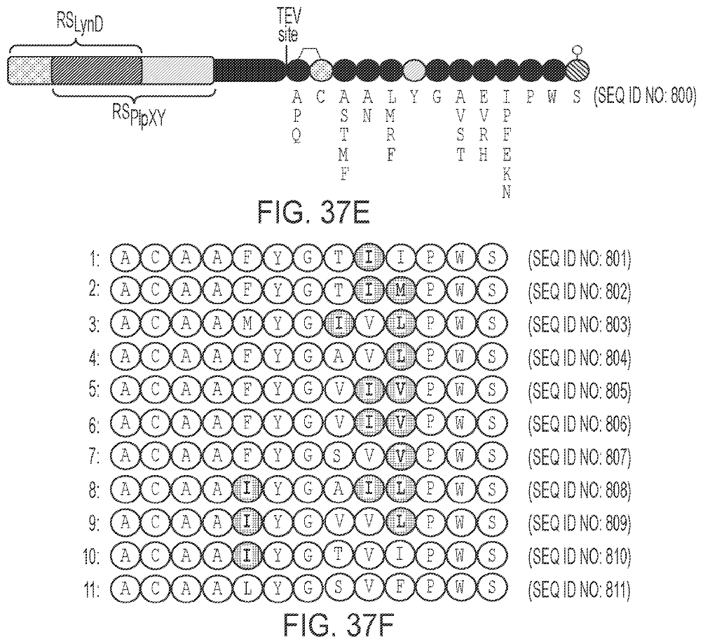

To engineer a RiPP, e.g., so as to include a particular set of post-translational modifications on a peptide having a particular amino acid sequence, the protein modification enzymes that facilitate the particular set of post-translational modifications are first identified. Consensus leader RS sequences for each leader-dependent enzyme are then compiled. Each leader RS sequence is then incorporated (e.g., by encoding in a nucleic acid sequence to be translated into the RiPP) into the leader sequence of the engineered RiPP. In embodiments in which one or all of the RS sequences for a given engineered RiPP have constraints on the distance between the RS and the amino acid(s) to be modified, each RS is placed in the leader sequence according to its respective constraint(s). An optimized leader sequence can be identified by screening candidate leaders and calculating a position score (e.g. by quantifying the amount of peptide having the desired modification pattern for each candidate leader sequence and identifying the leader sequence generating the highest yield of modified peptide). A non-limiting example of this screening process to identify optimized leader sequences is demonstrated in FIGS. 10A-10E and in FIG. 37D. The engineered RiPP is then expressed in a host cell concurrently with the protein modification enzymes, thereby synthesizing the engineered RiPP comprising the combination of post-translational modifications. In some embodiments, the engineered RiPP is expressed from a plasmid comprising a nucleic acid sequence encoding the leader and core amino acid sequence of the RiPP. In some embodiments, the protein modification enzymes are expressed from a plasmid or a set of plasmids comprising nucleic acid sequences encoding the enzymes. In some embodiments, the engineered RiPP and protein modification enzymes are expressed from a bacterial genome, such as an E. coli Marionette genome. In some embodiments, the engineered RiPP is expressed under the control of an inducible promoter. In some embodiments, each protein modification enzyme is expressed under the control of independently inducible promoters (i.e., each enzyme is controlled by an orthogonal promoter).

The RiPP engineering method provided herein enables the synthesis of a given peptide comprising a particular amino acid sequence with a specific combination of post-translational modifications. Biosynthesis using engineered RiPPs, rather than chemical or other conventional synthesis mechanisms, has one or more benefits, including but not limited to increased yield, decreased cost, and decreased complexity of the synthesis relative to alternative synthesis methods (e.g., chemical synthesis).

To engineer a RiPP, it may also be desirable to build a library of RiPPs to be screened with a particular protein modification enzyme or a particular combination of protein modification enzymes to identify preferred RiPPs (e.g., having a particular desired property or combination of properties) that comprise the desired post-translational modifications. Degenerate peptide libraries (i.e., libraries in which each amino acid of each member of the library is chosen randomly from all 20 natural amino acid options) can be designed, but have the disadvantage of being too large to be screened by conventional means (or in some instances are too large to be synthesized). For example, a degenerate library of peptides of 8 amino acids in length comprises peptides with 2.56×10¹⁰ distinct amino acid sequences, a number which is impossible or unfeasible to synthesize and/or screen. Such libraries are either impossible or unfeasible to synthesize and/or screen based on cost (sequencing, materials/reagents, etc.), time, or other considerations. As such, provided herein are libraries of RiPPs comprising a plurality of peptide members defined by a particular amino acid sequence motif. A library of RiPPs, in some embodiments, comprises peptides that are each 5-100 amino acids (e.g., 10-100, 10-90, 10-80, 10-70, 10-60, 10-50, 10-40, 10-30, 10-20, 20-100, 20-90, 20-80, 20-70, 20-60, 20-50, 20-40, 20-30, 30-100, 30-90, 30-80, 30-70, 30-60, 30-50, 30-40, 40-100, 40-90, 40-80, 40-70, 40-60, 40-50, 50-100, 50-90, 50-80, 50-70, 50-60, or any range or combination thereof) in length. A library, in some embodiments, comprises peptides that are each defined by a particular amino acid motif X₁X₂X₃X₄ . . . Xₙ, wherein n is the number of amino acids within the peptide (i.e., the length of the peptide), wherein each of X₁-Xₙ is independently chosen from 1, 2, 3, 4, 5, 6, 7, 8, 9, 10, 11, 12, 13, 14, 15, 16, 17, 18, 19, or 20 amino acids, and wherein at least one of X₁-Xₙ (e.g., 1, 2, 3, 4, 5, 6, 7, 8, 9, or all of X₁-Xₙ) is chosen from fewer than 20 amino acids. In some embodiments, at least one of X₁-Xₙ is restricted to a single amino acid. As a non-limiting example, X₁ may be chosen from 3 amino acids, X₂ may be chosen from 7 amino acids, X₃ may be chosen from 2 amino acids, and so on. In some embodiments, the amino acid motif X₁X₂X₃X₄ . . . Xₙ is determined to be susceptible to modification by 1, 2, 3, 4, 5, 6, 7, 8, or more distinct protein modification enzymes. In some embodiments, the plurality of peptides of the library do not have random amino acid sequences.

In some embodiments, a library comprises peptides defined by a particular amino acid motif determined to be susceptible to modification by 1, 2, 3, 4, 5, 6, 7, 8, or more distinct protein modification enzymes. In some embodiments, less than 100% (e.g., less than 95%, less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less than 5%, less than 4%, less than 3%, less than 2%, or less than 1%) of the members of the peptide library are capable of being fully modified by the protein modification enzymes to which the amino acid motif was determined to be susceptible. In some embodiments, at least 1% (e.g., at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%) of the members of the peptide library are capable of being fully modified by the protein modification enzymes to which the amino acid motif was determined to be susceptible.

In some embodiments, each member of a library disclosed herein comprises a SUMO tag. In some embodiments, each member of a library disclosed herein comprises a SUMO tag at its 5′ end. In some embodiments, each member of a library disclosed herein comprises a SUMO tag at its 3′ end. In some embodiments, each member of a library disclosed herein comprises a SUMO tag and a histidine tag at its 5′ end (e.g., the member comprises the structure [histidine tag]-[SUMO tag]-peptide or [SUMO tag]-[histidine tag]-peptide). In some embodiments, each member of a library disclosed herein comprises a SUMO tag and a histidine tag at its 3′ end (e.g., the member comprises the structure peptide-[histidine tag]-[SUMO tag] or peptide-[SUMO tag]-[histidine tag]-peptide). In some embodiments, each member of a library disclosed herein comprises a SUMO tag and a histidine tag at its 5′ end or at its 3′ end. In some embodiments, a histidine tag is a hexahistidine tag. In some embodiments, each member of a library disclosed herein comprises a tobacco etch virus protease (TEVp) cleavage site, or each member comprises two TEVp cleavage sites. In some embodiments, each member of a library disclosed herein comprises a TEVp cleavage site in between a RiPP peptide and a SUMO tag (e.g., the member comprises the structure peptide-[TEVp site]-[SUMO tag] or [SUMO tag]-[TEVp site]-peptide).

In some embodiments, a plurality of host cells comprises a library of peptides disclosed herein. In some embodiments, each host cell comprises a peptide of the library (e.g., each host cell comprises a peptide of the library and the peptide comprised by each host cell is independent of the peptides comprised by each other host cell). In some embodiments, each host cell is a bacterial cell. In some embodiments, each host cell comprises a nucleic acid sequence encoding the peptide. In some embodiments, each host cell further comprises a protein modifying enzyme. In some embodiments, the protein modifying enzyme is encoded by a nucleic acid sequence comprised by the host cell.

In some embodiments, a library is synthesized in a plurality of host cells. For example, in some embodiments, each member of the library is synthesized in a separate host cell. In some embodiments, each host cell is a bacterial cell. In some embodiments, a library is synthesized in a population of bacteria. In some embodiments, each bacterium of the population expresses a single member of the library. In some embodiments, each member of the library is synthesized in a host cell in which one or more protein modifying enzymes are also expressed.

In some embodiments, a library is capable of being screened by methods disclosed herein (e.g., using split-intein based selection). In some embodiments, screening of a library disclosed herein identifies one or more peptides with a desired functional property (e.g., a desired biological property). In some embodiments, screening of a library disclosed herein identifies one or more peptides with antimicrobial activity. In some embodiments, screening of a library disclosed herein identifies one or more peptides with binding activity to a target protein.

### Target Proteins

The target protein may be any protein of interest. In some embodiments, a target protein is a cell surface receptor, antigen, transmembrane protein, glycoprotein, glycolipid or any other cell surface macromolecule. In some embodiments, the target protein is a viral protein or a fragment thereof. In some embodiments, the target protein comprises a receptor binding domain (RBD) from a coronavirus protein. In some embodiments, the coronavirus is 229E (alpha coronavirus), NL63 (alpha coronavirus), OC43 (beta coronavirus), HKU1 (beta coronavirus), MERS-CoV (the beta coronavirus that causes Middle East Respiratory Syndrome, or MERS), SARS-CoV (the beta coronavirus that causes severe acute respiratory syndrome, or SARS), or SARS-CoV-2 (the novel coronavirus that causes coronavirus disease 2019, or COVID-19). In some embodiments, the target protein is a bacterial protein or a fragment thereof. In some embodiments, the target protein is a bacterial enzyme. In some embodiments, the target protein is a bacterial outer-membrane protein. In some embodiments, the target protein is a bacterial toxin. In some embodiments, the target protein is a bacterial structural protein. In some embodiments, the target protein is a bacterial polymerase. In some embodiments, the target protein is a bacterial transcription regulator.

In some embodiments, the target protein is SARS-CoV-2 receptor binding domain (RBD) of the Spike protein. Spike protein is a surface glycoprotein that binds to angiotensin I converting enzyme 2 (ACE2) to promote viral entry. The al helix of ACE2 makes most of the binding contacts with the RBD and is provided as SEQ ID NO: 72.

In some embodiments, the target protein comprises a sequence that is at least 60%, at least 70%, at least 80%, at least 90%, at least 91%, at least 92%, at least 93%, at least 94%, at least 95%, at least 96%, at least 97%, at least 98%, or at least 99% identical to SEQ ID NO: 71 (RBD). In some embodiments, the target protein comprises the amino acid sequence of SEQ ID NO: 71.

In some embodiments, the target protein comprises a sequence that is at least 60%, at least 70%, at least 80%, at least 90%, at least 91%, at least 92%, at least 93%, at least 94%, at least 95%, at least 96%, at least 97%, at least 98%, or at least 99% identical to SEQ ID NO: 72 (al helix of ACE2). In some embodiments, the target protein comprises the amino acid sequence of SEQ ID NO: 72.

Non-limiting examples of known cellular receptors include ACVR2A, EGFR/HER1, HER2/ERBB2, ERBB3/HER3, CD32a/FCGR2A/Fc gamma RIIa, CD32b/FCGR2B/Fc gamma RIIb, CD16a/Fc gamma RIIIa, CD16b/Fc gamma RIII, CD155/PVR, TNFR1/TNFRSF1A/CD120a, TNFR2/TNFRSF1B/CD120b, 4-1BB/TNFRSF9/CD137, TRAIL R2/CD262/TNFRSF10B, TRAIL R4/CD264/TNFRSF10D, TNFRSF11A, TRAIL R1/CD261/TNFRSF10A, TRAILR3/TNFRSF10C, TACI/TNFRSF13B(CD267) HVEM/TNFRSF14/CD270, BCMA/TNFRSF17/CD269, GITR/TNFRSF18/CD357, FGFR2/CD332, CD23/FCER2, FCRL1/FCRH1, TIM-3/HAVCR2, IL1RL1/IL-1 R4, IL17RA/IL-17RA/CD217, IL-4R/CD124, IL7R/IL-7R/CD127, TrkA/NTRK1, PDGFRB/CD140b, TREM-2/TREM2, ACVR2B/Activin RIIB, FCGRT & B2M, CD89/FCAR, IL3RA/CD123, IGF1R/CD221/IGF-I R, Insulin Receptor/INSR/CD220, LILRB2/ILT4/LIR-2, VEGFR2/KDR/Flk-1/CD309, MCSF Receptor/CSF1R/CD115, EPHA3/Eph Receptor A3, CD16-2/FCGR4, FcERI/FCER1A, TIM-1/KIM-1/HACVR, IL6R/IL-6R/CD126, LILRB4/CD85k/ILT3, IL2RA/IL-2RA/CD25, CD122/IL-2RB, LDLR/LDL R/LDL Receptor, CD112/Nectin-2/PVRL2, and TFRC/CD71.

A peptide described herein may have a particular binding affinity for a target protein. Binding affinity is the apparent association constant or K_{A}. The K_{A} is the reciprocal of the dissociation constant (K_{D}). The peptides identified by the methods described herein may have a binding affinity (K_{D}) of at least 10⁻⁵, 10⁻⁶, 10⁻⁷, 10⁻⁸, 10⁻⁹, 10⁻¹⁰ M, or lower for a target protein. An increased binding affinity corresponds to a decreased K_{D}. Higher affinity binding of a peptide for a first protein relative to a second protein can be indicated by a higher K_{A} (or a smaller numerical value K_{D}) for binding the first protein than the K_{A} (or numerical value K_{D}) for binding the second protein. In such cases, the peptide has specificity for the first protein (e.g., a first protein in a first conformation or mimic thereof) relative to the second protein (e.g., the same first protein in a second conformation or mimic thereof; or a second protein). In some embodiments, the peptides described herein have a higher binding affinity (a higher K_{A} or smaller K_{D}) to an appropriate protein as compared to the binding affinity of the same type of peptide produced using naturally occurring secretion signal peptides. Differences in binding affinity (e.g., for specificity or other comparisons) can be at least 1.5, 2, 3, 4, 5, 10, 15, 20, 37.5, 50, 70, 80, 91, 100, 500, 1000, 10,000 or 10⁵ fold. In some embodiments, any of the peptides produced as provided herein may be further affinity matured to increase the binding affinity of the peptide to the target protein or epitope thereof.

Binding affinity (or binding specificity) can be determined by a variety of methods including equilibrium dialysis, equilibrium binding, gel filtration, ELISA, surface plasmon resonance, or spectroscopy (e.g., using a fluorescence assay). Non-limiting exemplary conditions for evaluating binding affinity are in HBS-P buffer (10 mM HEPES pH7.4, 150 mM NaCl, 0.005% (v/v) Surfactant P20). These techniques can be used to measure the concentration of bound binding protein as a function of target protein concentration. The concentration of bound binding protein ([Bound]) is generally related to the concentration of free target protein ([Free]) by the following equation:

[Bound]=[Free]/(Kd+[Free])

It is not always necessary to make an exact determination of K_{A}, though, since sometimes it is sufficient to obtain a quantitative measurement of affinity, e.g., determined using a method such as ELISA, FACS analysis or magnetic immunoprecipitation, which is proportional to K_{A}, and thus can be used for comparisons, such as determining whether a higher affinity is, e.g., 2-fold higher, to obtain a qualitative measurement of affinity, or to obtain an inference of affinity, e.g., by activity in a functional assay, e.g., an in vitro or in vivo assay.

In some embodiments, a peptide disclosed herein or identified through the methods disclosed herein decreases the binding affinity of a target peptide with a naturally occurring cognate binding partner. In some embodiments, a peptide disclosed herein or identified through the methods disclosed herein decreases the binding affinity of a target peptide with a naturally occurring cognate binding partner by at least 10%, at least 20%, at least 30%, at least 40%, at least 50%, at least 60%, at least 70%, at least 80%, at least 90%, at least 95%, or at least 99%.

### Host Cells

Aspects of the present disclosure provide host cells comprising any of the nucleic acids, fusion proteins, peptides, enzymes, selection markers and components of the split intein-based systems disclosed herein. In some embodiments, a host cell is a eukaryotic cell. In some embodiments, a host cell is a prokaryotic cell. In some embodiments, a host cell is a bacterial cell. In some embodiments, a host cell is an E. coli cell. As one of ordinary skill in the art would appreciate, components of the split intein-based systems disclosed herein may be selected based on the type of host cell used.

A nucleic acid may encode any of the fusion proteins, peptides, enzymes, selection markers and components of the split intein-based systems disclosed herein. As used herein, a heterologous nucleic acid is one that is introduced into a host cell. A nucleic acid, generally, is at least two nucleotides covalently linked together, and in some instances, may contain phosphodiester bonds (e.g., a phosphodiester “backbone”). A nucleic acid is considered “engineered” if it does not occur in nature. Examples of engineered nucleic acids include recombinant nucleic acids and synthetic nucleic acids.

Nucleic acids encoding any of the fusion proteins, peptides, enzymes, selection markers and components of the split intein-based system described herein may be introduced into a host cell using any known methods, including but not limited to chemical transfection, viral transduction and electroporation. In some embodiments, one or more nucleic acids that are introduced into a host cell integrate into the host cell genome; in some embodiments, one or more nucleic acids that are introduced in a host cell do not integrate into the host cell genome. The nucleic acids described herein may encode one or more of the fusion proteins, peptides, enzymes, selection markers and components of the split intein-based system disclosed herein. In some embodiments, a nucleic acid comprises a sequence that is at least 60%, at least 70%, at least 80%, at least 90%, at least 91%, at least 92%, at least 93%, at least 94%, at least 95%, at least 96%, at least 97%, at least 98%, or at least 99% identical to SEQ ID NOs: 47-66 or 73-79, a nucleic acid sequence in Table 3, or a nucleic acid sequence disclosed herein. In some embodiments, a nucleic acid comprises a nucleotide sequence of any one of SEQ ID NOs: 47-66 or 73-79, a nucleic acid sequence in Table 3, or a nucleic acid sequence disclosed herein. Any of the plasmids disclosed herein may be used.

It should be understood the methods of identifying peptides disclosed herein may or may not use host cells. In some embodiments, a split intein-based system disclosed herein is not used in a host cell. For example, in vitro methods comprising incubating a split intein-based system disclosed herein in a reaction vessel under suitable conditions is encompassed by the present disclosure.

### Kits

Any of the host cells, nucleic acids, fusion proteins, peptides, enzymes, selection markers and components of the split intein-based systems disclosed herein, in some embodiments, may be assembled into pharmaceutical or diagnostic or research kits to facilitate their use in therapeutic, diagnostic or research applications. A kit may include one or more containers housing the components of the disclosure and instructions for use. Specifically, such kits may include one or more agents described herein, along with instructions describing the intended application and the proper use of these agents. In certain embodiments, agents in a kit may be in a pharmaceutical formulation and dosage suitable for a particular application and for a method of administration of the agents. Kits for research purposes may contain the components in appropriate concentrations or quantities for running various experiments.

In some embodiments, the instant disclosure relates to a kit for identifying a peptide that binds a target protein, the kit comprising a container housing any of the host cells, nucleic acids, fusion proteins, peptides, enzymes, and components of the split intein-based systems disclosed herein. In some embodiments, the kit further comprises instructions for identifying the peptide and/or performing the split intein-based selection.

In some embodiments, the instant disclosure relates to a kit comprising a container housing any of the nucleic acids disclosed herein. In some embodiments, the kit comprises a container housing a nucleic acid that comprises a sequence that is at least 60%, at least 70%, at least 80%, at least 90%, at least 91%, at least 92%, at least 93%, at least 94%, at least 95%, at least 96%, at least 97%, at least 98%, or at least 99% identical to SEQ ID NOs: 47-66 or 73-79, a nucleic acid sequence in Table 3, or a nucleic acid sequence disclosed herein; or that comprises the nucleotide sequence of any one of SEQ ID NOs: 47-66 or 73-79, a nucleic acid sequence in Table 3, or a nucleic acid sequence disclosed herein. In some embodiments, the instant disclosure relates to a kit comprising a container housing any of the peptides disclosed herein. In some embodiments, the kit comprises a container housing a peptide that comprises a sequence that is at least 60%, at least 70%, at least 80%, at least 90%, at least 91%, at least 92%, at least 93%, at least 94%, at least 95%, at least 96%, at least 97%, at least 98%, or at least 99% identical to SEQ ID NOs: 6-25 or 26-45, an amino acid sequence in Table 3 or any amino acid sequence disclosed herein, including fragments thereof; or that comprises the amino acid sequence of any one of SEQ ID NOs: 6-25 or 26-45, an amino acid sequence in Table 3 or any amino acid sequence disclosed herein, including fragments thereof. In addition, kits of the disclosure may include instructions, a negative and/or positive control, containers, diluents and buffers for the sample, sample preparation tubes and a printed or electronic table of reference peptide sequences for sequence comparisons.

The kit may be designed to facilitate use of the methods described herein by researchers and can take many forms. Each of the compositions of the kit, where applicable, may be provided in liquid form (e.g., in solution), or in solid form, (e.g., a dry powder). In certain cases, some of the compositions may be constitutable (e.g., reconstitutable) or otherwise processable (e.g., to an active form), for example, by the addition of a suitable solvent or other species (for example, water or a cell culture medium), which may or may not be provided with the kit. As used herein, “instructions” can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions may be in a form prescribed by a governmental agency regulating the manufacture, use or sale of pharmaceuticals or biological products, which instructions can also reflect approval by the agency of manufacture, use or sale for animal administration. The kit may contain any one or more of the components described herein in one or more containers. As an example, in one embodiment, the kit may include instructions for mixing one or more components of the kit and/or isolating and mixing a sample and applying to a subject. The kit may include a container housing agents described herein. The agents may be in the form of a liquid, gel or solid (powder). The agents may be prepared sterilely, packaged in syringe and shipped refrigerated. Alternatively, it may be housed in a vial or other container for storage. A second container may have other agents prepared sterilely. Alternatively, the kit may include the active agents premixed and shipped in a syringe, vial, tube, or other container. The kit may have one or more or all of the components required to administer the agents to an animal, such as a syringe, topical application devices, or IV needle tubing and bag, particularly in the case of the kits for producing specific somatic animal models.

The kit may have a variety of forms, such as a blister pouch, a shrink-wrapped pouch, a vacuum sealable pouch, a sealable thermoformed tray, or a similar pouch or tray form, with the accessories loosely packed within the pouch, one or more tubes, containers, a box or a bag. The kit may be sterilized after the accessories are added, thereby allowing the individual accessories in the container to be otherwise unwrapped. The kits can be sterilized using any appropriate sterilization techniques, such as radiation sterilization, heat sterilization, or other sterilization methods known in the art. The kit may also include other components, depending on the specific application, for example, containers, cell media, salts, buffers, reagents, syringes, needles, a fabric, such as gauze, for applying or removing a disinfecting agent, disposable gloves, a support for the agents prior to administration etc.

### Pharmaceutical Compositions and Uses Thereof

Any of the peptides (e.g., modified peptides) disclosed herein or identified by a method disclosed herein may be formulated in a pharmaceutical composition for administration to a subject. As used herein, a subject is a human, non-human primate, cow, horse, pig, sheep, goat, dog, cat, or rodent. In all embodiments, human subjects are preferred.

In some embodiments, the subject is a suspected of having a disease or has previously been diagnosed as having a disease. In some embodiments, the subject is a human suspected of having a disease, or a human having been previously diagnosed as having a disease. Methods for identifying subjects suspected of having a disease may include physical examination, subject's family medical history, subject's medical history, biopsy, viral tests (e.g., nasal swabs), antibody tests (e.g., serological testing), or a number of imaging technologies such as ultrasonography, X-ray imaging, computed tomography, magnetic resonance imaging, magnetic resonance spectroscopy, or positron emission tomography.

In some embodiments, the subject is suspected of having or has previously been diagnosed as having an infectious disease (e.g., a disease caused by a pathogen and/or virus). As a non-limiting example, the subject may have coronavirus disease 2019 (COVID-19), which is an infectious disease. COVID-19 is caused by severe acute respiratory syndrome coronavirus 2 (SARS-CoV-2). SARS-CoV-2 may be diagnosed using any suitable method including nasopharyngeal swabs and serology testing for antibodies against coronavirus.

In some embodiments, the subject is suspected of having or has previously been diagnosed as having cancer. The term “cancer” refers to a class of diseases characterized by the development of abnormal cells that proliferate uncontrollably and have the ability to infiltrate and destroy normal body tissues. See, e.g., Stedman's Medical Dictionary, 25th ed.; Hensyl ed.; Williams & Wilkins: Philadelphia, 1990. Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenstram's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

In some embodiments, the subject is suspected of having or has previously been diagnosed as having a bacterial infection (e.g., an infection caused by a pathogenic bacterium). Exemplary bacterial infections include, but are not limited to, pulmonary infections (e.g., upper respiratory infection or lower respiratory infections), urinary tract infections, skin infections (e.g., bacterial cellulitis), sexually transmitted infections, neurological infections (e.g., bacterial encephalitis, bacterial meningitis), cardiac infections (e.g., bacterial endocarditis, bacterial myocarditis, or bacterial pericarditis), gastrointestinal infections (e.g., gastric infections, bacterial gastroenteritis, bacterial pharyngitis), bacterial vaginosis, and Lyme disease. Bacterial infections can be caused by any bacterium, including, but not limited to, Gram-positive bacteria, Gram-negative bacteria, Streptococcus pneumoniae, Haemophilus species, Staphylococcus aureus, Mycobacterium tuberculosis, methicillin-resistant S. aureus, non-typhoidal Salmonella species, Salmonella typhi, Bacillus cereus, Clostridium perfringens, Clostridium botulinum, Escherichia coli (ETEC, EPEC, EHEC, EAEC, EIEC), Salmonella sp., Shigella sp., Campylobacter sp., Yersinia enterocolitica, Clostridium difficile, Vibrio cholerae, Vibrio parahemolyticus, Listeria monocytogenes, Aeromonas hydrophila, Plesiomonas sp., Neisseria meningitidis, Streptococcus pneumoniae, Haemophilus influenzae, Neisseria gonorrhoeae, Chlamydia trachomatis, Treponema pallidum, Borrelia burgdorferi, Vibrio cholerae, Clostridium tetani, and Bacillus anthracis.

A “plurality” of elements, as used throughout the application refers to two or more of the elements.

The peptides (e.g., modified peptides) of the invention are administered to the subject in an effective amount for detecting or modulating protein (e.g., enzyme) activity. An “effective amount”, for instance, is an amount required to confer therapeutic effect on a subject, either alone or in combination with at least one other active agent. The effective amount of a peptide of the invention described herein may vary depending upon the specific peptide used, the mode of delivery of the peptide, and whether it is used alone or in combination. The effective amount for any particular application can also vary depending on such factors as the disease being assessed or treated, the particular peptide being administered, the size of the subject, or the severity of the disease or condition as well as the detection method. One of ordinary skill in the art can empirically determine the effective amount of a particular molecule of the invention without necessitating undue experimentation. Combined with the teachings provided herein, by choosing among the various active peptides and weighing factors such as potency, relative bioavailability, patient body weight, severity of adverse side-effects and preferred mode of administration, an effective regimen can be planned.

Pharmaceutical compositions of the present invention comprise an effective amount of one or more agents, dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences (1990), incorporated herein by reference). Except insofar as any conventional carrier is incompatible with the active ingredient, its use in the therapeutic or pharmaceutical compositions is contemplated. The agent may comprise different types of carriers depending on whether it is to be administered in solid, liquid or aerosol form, and whether it need to be sterile for such routes of administration as injection.

General considerations in the formulation and/or manufacture of pharmaceutical agents, such as compositions comprising any of the engineered cells disclosed herein, may be found, for example, in Remington: The Science and Practice of Pharmacy 21st ed., Lippincott Williams & Wilkins, 2005 (incorporated herein by reference in its entirety).

Suitable routes of administration include, for example, parenteral routes such as intravenous, intrathecal, parenchymal, or intraventricular injection.

### EXAMPLES

### Example 1: Plasmid Design for Split Intein-Based RiPP Selections

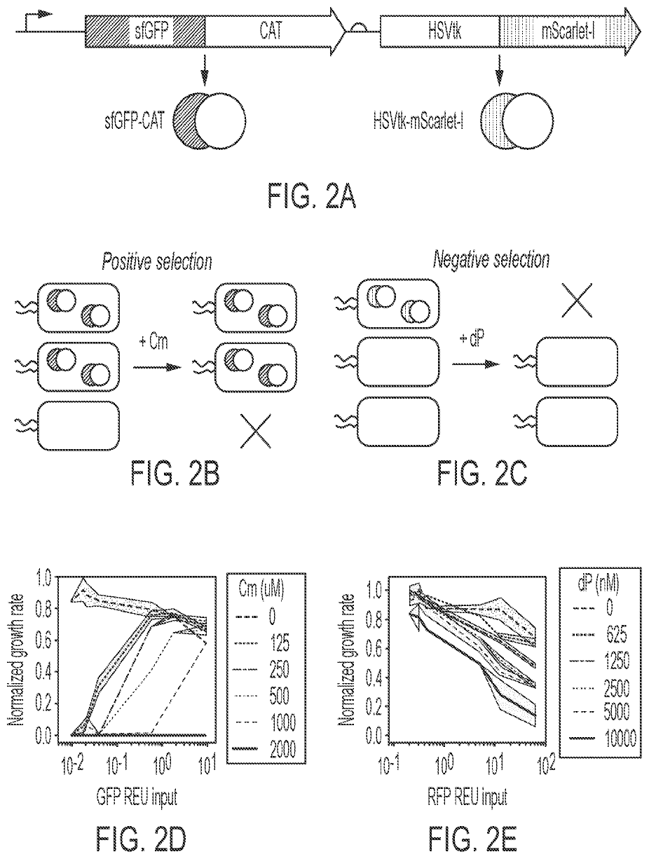

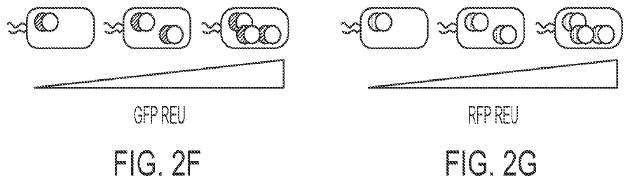

A three plasmid system was used to conduct selection experiments. All plasmids are low-medium copy number variants previously characterized¹: the “peptide plasmid” is a pSC101 backbone with an ampicillin resistance cassette (working concentration of 100 ng/uL) and contains a Type IIs restriction site for insertion of RiPP/peptide sequences N-terminal to one half of the split intein/sigma factor under control of an inducible T5 lacO promoter (maximally induced with 1 mM IPTG). The “modifying enzyme plasmid” is a p15A backbone with a spectinomycin resistance cassette (working concentration of 50 ng/uL) and contains a Type IIs restriction site for inserting cognate RiPP modifying enzymes under control of an inducible pCym promoter (maximally induced with 100 uM cumate). The “selection plasmid” is a ColE1 backbone with a kanamycin resistance cassette (working concentration of 50 ng/uL) and contains two regions of expression. The first is a C-terminal fusion of the SARS-CoV-2 receptor binding domain (RBD) of the Spike protein² to the other half of the split intein-sigma factor. The second expression region contains two open reading frames downstream of the ECF20_992 promoter. The first is a sfGFP-cat gene for expression of superfolder-green fluorescent protein (sfGFP) and a chloramphenicol acetyltransferase (CAT) and the second is hsvTK-mScarlet-I gene for expression of the red fluorescent protein mScarlet-I and, when in the presence of a nucleoside analog, the toxic gene product, herpes simplex virus thymidine kinase (HsvTK) 3 (FIG. 2A).

The three plasmid system allows for flexible selection methods. Inducible expression of the peptide and modifying enzyme plasmids results in production of modified RiPP libraries with C-terminal fusions to the split intein machinery. RiPPs that are able to bind to the target (in this case, the RBD) lead to productive intein association and splicing 4 of the split sigma factor, which induces expression of the selection cassettes. For positive selection of binders, increasing concentrations of chloramphenicol (cm) can be used to enrich for target binders (in this case, an RBD-intein fusion) that produce increasing amounts of CAT (FIG. 2B, FIG. 2D, and FIG. 2F). For negative selection of binders, increasing concentrations of nucleotide 6-(β-D-2-deoxyribofuranosyl)-3,4-dihydro8H-pyrimido [4,5-c][1,2] oxazin-7-one (dP) can be used to deplete target binders (in this case, a Mdm2-intein fusion; note any off-target protein fusion is suitable) that produce increasing amounts of HsvTK (FIG. 2C, FIG. 2E, and FIG. 2G).

For the generation of this initial round of RBD hits, a negative selection was not implemented. Current and future selections will utilize positive and negative selections in consecutive, discrete rounds to best evolve RiPP libraries toward high affinity and specific binders to the RBD.

### Example 2: Identification of RiPP Binders of RBD

### Design and Construction of RiPP Libraries and Cognate Modifying Enzymes

Five libraries were designed based on in-house understanding of RiPP biosynthetic constraints, (FIG. 3). Library 1 contains recognition sites (RS) for the enzymes ProcM and LynD, which install lanthionines and thiazolines, respectively. Library 2 contains RS for the enzymes TgnB and LynD, which install ester linkages and thiazolines, respectively. Libraries 3-5 contain the RS for the enzyme PapB, which installs thioethers. The predicted cyclization topologies and amino acid degeneracy are outlined for each library in FIG. 3.

Library sizes were as indicated in Table 2 based on serial dilutions and counting colony forming units (CFU)/mL.

TABLE 2
Library sizes

| library | core mod | size |
|---|---|---|
| 1 | procM | 6E+07 |
| 2 | tgnB | 1E+07 |
| 3 | papB | 1E+07 |
| 4 | papB | 1E+06 |
| 5 | papB | 1E+07 |

### Selection Methods for Generation of Pilot Hits

Appropriate antibiotics were used at every stage for plasmid propagation, as detailed above. Inducers were used at maximum concentration where indicated, as detailed above. Transformation efficiencies were recorded via serial dilution and CFU/mL counts. Libraries were miniprepped and transformed into separate electrocompetent strains of E. coli Marionette-Clo⁵ containing cognate modifying enzyme and selection constructs (transformation efficiencies >10⁸ CFU/mL). After a one-hour outgrowth, strains were diluted 1:50 for plasmid outgrowth and induction of library peptides and modifying enzymes. This culture was grown overnight at 30° C., with shaking at 250 RPM.

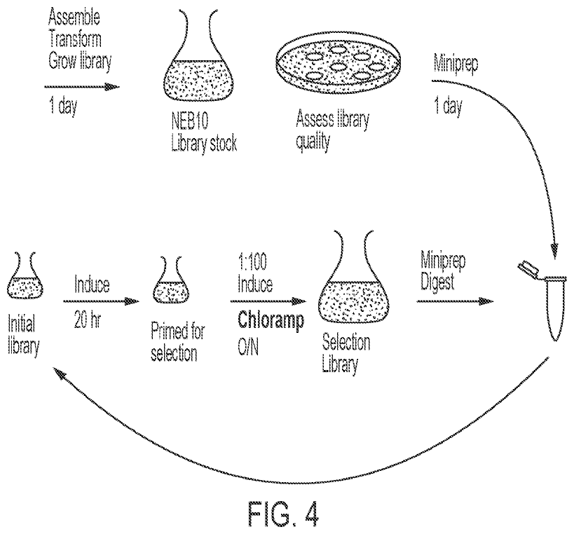

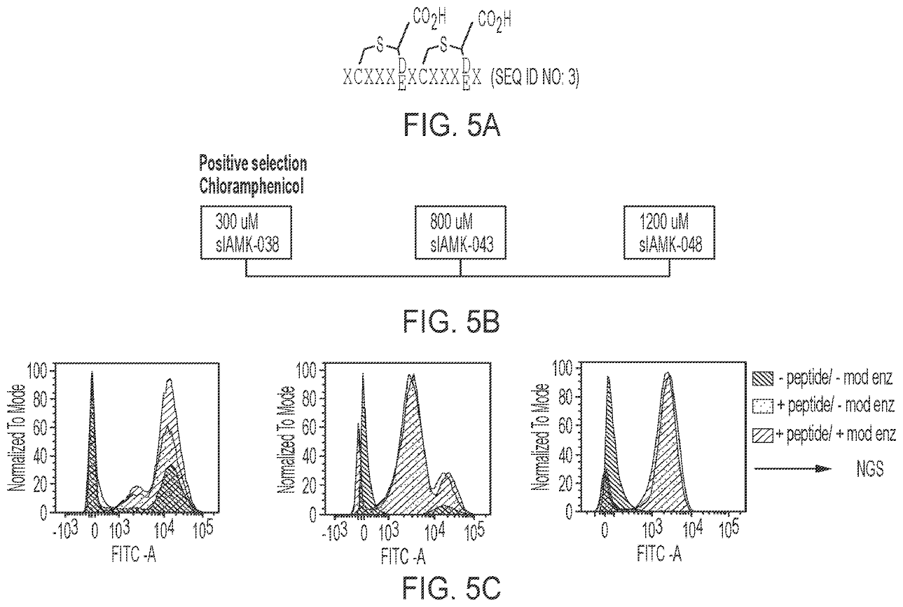

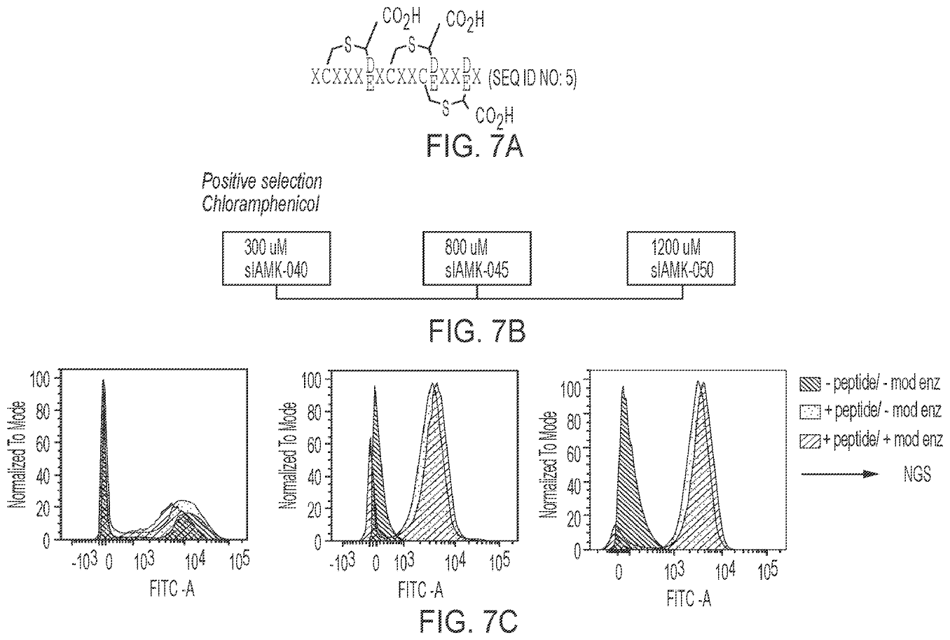

After overnight growth, libraries were diluted 1 mL in 100 mL TB medium in inducing conditions. Selections were grown at 30° C. for 20 hours, 250 RPM. 4 mL of each selection was miniprepped and modifying enzyme/selection plasmids were restriction digested using SacI/KpnI (NEB, per manufacturer's instructions). Resulting digests were column purified (Zymo) and re-transformed in strains containing modifying enzyme/selection plasmids. This step was done in order to eliminate escape mutants in the selection plasmid (for instance, mutations generating high-level, constitutive expression of cat-GFP; see FIGS. 5 and 7). FIG. 4 outlines the process graphically.

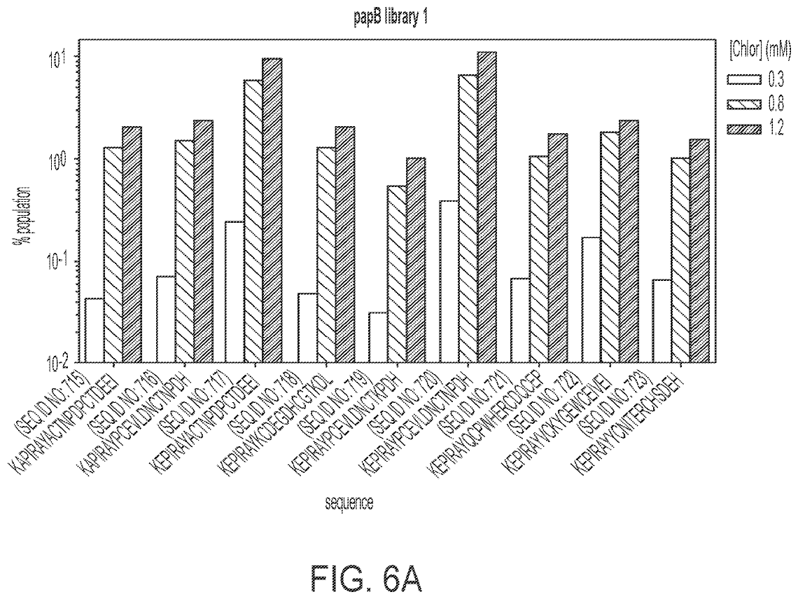

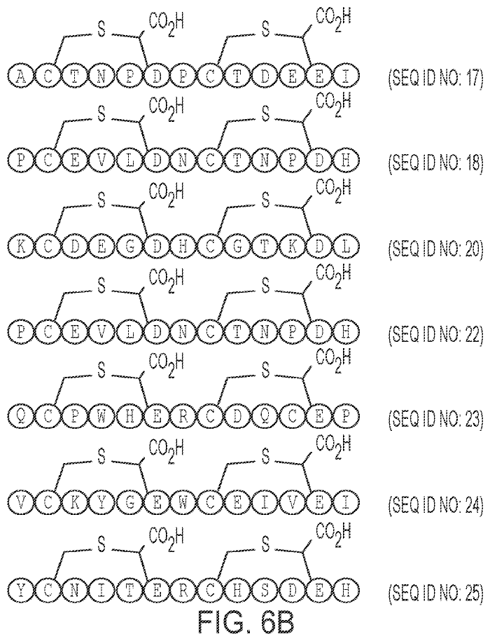

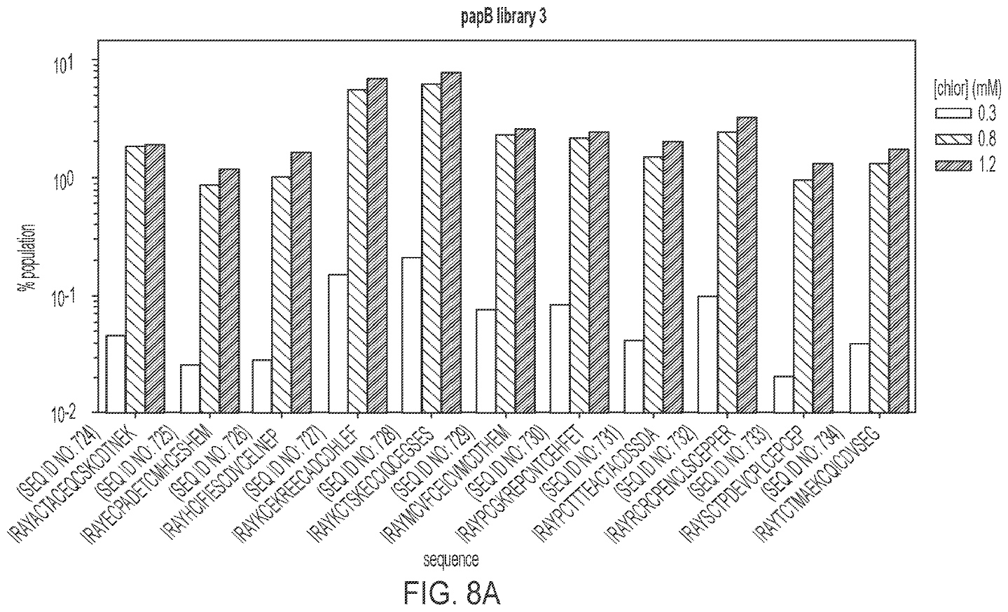

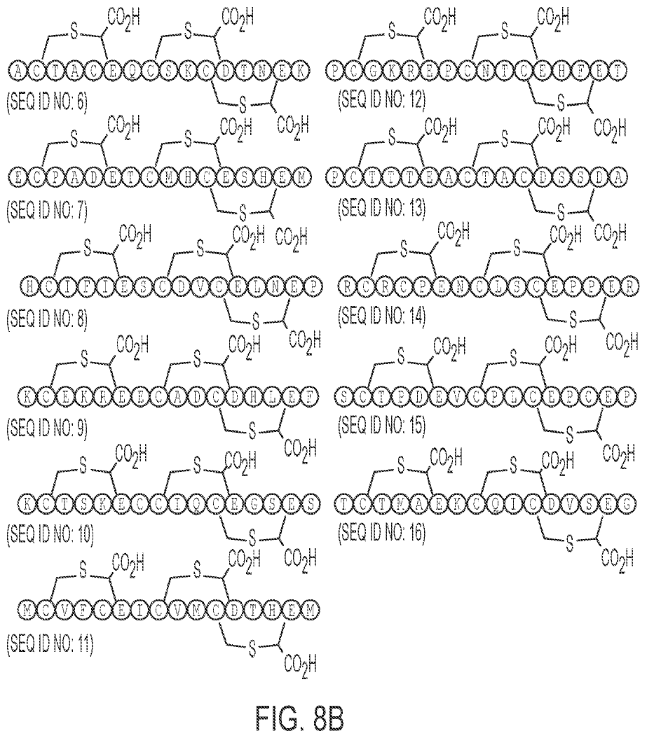

For this initial pilot screen, 3 rounds of positive selections were conducted, at 300, 800 and 1200 uM chloramphenicol. Cell populations were assessed via cytometry to observe shifts in REU values (FIGS. 5 and 7). Libraries 3 (FIGS. 5A, 5B, and 5C) and 5 (FIGS. 7A, 7B, and 7C) demonstrated ideal REU shifts over rounds of selection and were chosen for next-generation sequencing (NGS). Degenerate regions of the peptide library plasmid were amplified and submitted for Illumina sequencing (HiSeq) to generate quantitative reads of peptide populations. Peptide sequences that were enriched in iterative selection rounds and also comprised >1% of the final population are summarized in FIGS. 6A and 6B (Library 3) and FIGS. 8A and 8B (Library 5).

### Confirmation of Pilot Hits

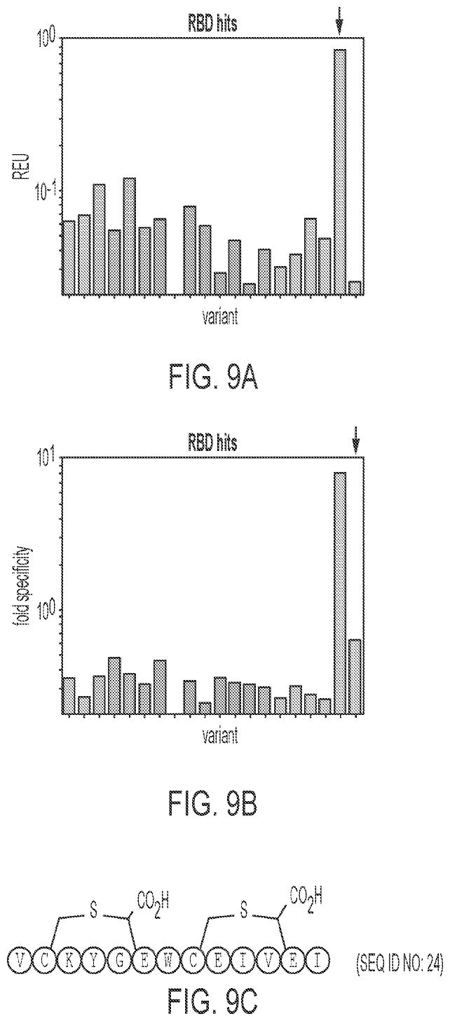

20 sequences were codon optimized, synthesized as gBlocks (IDT), and individually cloned into the peptide plasmid. These 20 peptide plasmids were co-transformed with the PapB modifying enzyme plasmid and either the RBD-intein or Mdm2-intein as target in the selection plasmid. After overnight induction of peptide/modifying enzyme at 30° C., cells were analyzed via cytometry and REU values determined (FIG. 9A). Fold specificity was determined by comparing the ratio of REU values of peptides either against RBD or Mdm2-intein fusions (FIG. 9B). One hit emerged as having high specificity for the RBD (FIG. 9C).

### REFERENCES FROM EXAMPLES 1 AND 2

1 Segall-Shapiro, T. H., Sontag, E. D. & Voigt, C. A. Engineered promoters enable constant gene expression at any copy number in bacteria. Nat. Biotechnol., doi:10.1038/nbt.4111 (2018).
2 Lan, J. et al. Structure of the SARS-CoV-2 spike receptor-binding domain bound to the ACE2 receptor. Nature 581, 215-220, doi:10.1038/s41586-020-2180-5 (2020).
3 Kawai-Noma, S. et al. Improvement of the dP-nucleoside-mediated herpes simplex virus thymidine kinase negative-selection system by manipulating dP metabolism genes. J Biosci Bioeng, doi:10.1016/j.jbiosc.2020.03.002 (2020).
4 Stevens, A. J. et al. Design of a Split Intein with Exceptional Protein Splicing Activity. J. Am. Chem. Soc. 138, 2162-2165, doi:10.1021/jacs.5b13528 (2016).
5 Meyer, A. J., Segall-Shapiro, T. H., Glassey, E., Zhang, J. & Voigt, C. A. Escherichia coli “Marionette” strains with 12 highly optimized small-molecule sensors. Nat. Chem. Biol., doi:10.1038/s41589-018-0168-3 (2018).

### Example 3: De Novo Design of Enzyme-Modified Peptides

Chemically-modified peptides are made by all kingdoms of life, where the enzymatic decorating and reshaping are critical for function. Peptides could be designed de novo by harnessing the modifying enzymes from the deluge of genomics, but it is difficult to extract the rules guiding their use and combination. In this Example, a model that captures the minimal specificity constraints was developed to use enzymes gleaned from microbial gene clusters encoding RiPPs (ribosomally-synthesized and post-translationally modified peptides). They include the recognition site (RS) sequence and restrictions on its placement in the precursor peptide and the tolerance to variability of the released core. The rule sets were empirically parameterized using a pipeline to construct and evaluate the activities of enzymes against hundreds of precursor peptide variants in Escherichia coli. This was applied to nine enzymes from eight RiPPs classes, including those for which there is little prior characterization (lactone macrocyclase, tyramine excisionase, glutamate heterocyclase, cysteine heterocyclase, glycosyltransferase, serine kinases, decarboxylase, and methyl transferase). The rules can be algorithmically combined to computationally design new-to-nature RiPPs, demonstrated by creating a 13-mer that combines excision, heterocyclization, and phosphorylation (PlpXY, LynD, ThcoK). Formalizing enzyme rules provides a foundation for retrosynthesis, where peptides and libraries could be designed to facilitate therapeutic discovery and diversification.

### INTRODUCTION

Across biology, peptides are chemically modified for diverse purposes, from enhancing antimicrobial potency to honing signaling specificity and nucleating inorganic materials [1-6]. In the pursuit of pharmaceutical or other applications, one would like to design patterns of modifications in a peptide, but this is challenging using total synthesis because routes are long and involve highly-functionalized and chiral molecules [7-9]. An alternative would be to encode the peptide as a gene that is expressed with enzymes that introduce the desired post-translational modifications (PTMs) [10-12]. The process of identifying a path to a target molecule is a form of retrosynthesis that requires knowing the rules by which enzymes can be combined to act on a peptide sequence [13].

Peptide secondary metabolites are often encoded in genomes as a RiPP where a precursor peptide is expressed that comprises a leader and core sequence [3]. An enzyme binds to a recognition site (RS) in the leader and modifies amino acid(s) in the core [14-16]. PTMs include the introduction of cycles, added moieties (e.g, methylation), or conversions (e.g., epimerization) [3, 17-19]. A leader can have up to three RSs, sometimes overlapping to save space [20-22]. Changing the distance dbetween the RS and the modified amino acid(s) can affect the efficiency and which amino acids are modified [22-31]. Some enzymes are more sensitive than others, likely due to flexibility or allostery [32-34]. Leader-independent “tailoring” enzymes add modifications before or after the proteolytic release of the core [3]. To date, up to eight modifying enzymes have been found to act on a single peptide (theiostrepton), but the number of modifications can be much larger (e.g., polytheonamide has 49 modifications by 7 enzymes) [22, 35, 36].

During evolution, core hypervariability around a PTM scaffold facilitates the exploration of functional space, for example to diversify antimicrobials against new threats [10, 11, 37-40]. By physically separating binding from catalysis, leader-dependent enzymes are highly tolerant to changes to the core sequence; typically, 40-90% of mutants are modified correctly [12, 16, 17, 19, 20, 26, 27, 31, 41-50]. The specificity of tailoring enzymes can vary, with some being sensitive to sequence or the peptide conformation and others being very broad, notably when they modify the termini [46, 51-55]. Taken together, the minimal rule set needed to repurpose an enzyme is: 1. the tolerance of the core sequence, and 2. the RS sequence and position constraints within the leader, if relevant (FIG. 10A).

Various approaches have been used to discern these rules. Importantly, when characterizing an enzyme for retrosynthesis, the constraints must be with respect to the chemistry performed and not function [42]. For example, in one study, only 41% of thiopeptide mutations that yielded the correct PTM also retained antibiotic activity [56]. While bioinformatics can be used to deduce the RS or enumerate core variability, drawing them from natural genomes implies functionality [57, 58]. Another approach is to evaluate the impact of mutants with libraries created though alanine-scanning, saturation mutagenesis, or core shuffling [42, 47, 59-66]. Billions can be evaluated using assays that screen for function or by panning for target binding [26, 47, 56, 62, 64, 67-72]. The throughput of chemical assays is more limited; electrospray ionization mass spectroscopy (ESI-MS) can characterize hundreds of variants [29, 33, 42, 73]. MALDI-MS and SAMDI-MS could scale to 10⁴ variants or more, but they are currently limited by peptide length and require additional expensive processing steps when automated [31, 50, 56, 74, 75].

Early work has combined enzymes from different pathways to build novel compounds, but typically, these have been sourced from the same RiPPs family [39, 55, 76]. Some tailoring enzymes will modify nearly any core and this observation has been used to incorporate methyltransferases, decarboxylation or epimerases into unrelated pathways [46, 55, 77]. Combining enzymes across RiPP classes has proven more difficult. In pioneering work, Mitchell and van der Donk showed that leader-dependent enzymes from sactipeptide, lanthipeptide, and heterocycloanthracin pathways could be combined by creating leader chimeras combining the RSs [74]. Along with a tailoring enzyme, this was used to make a new 32-mer lanthipeptide containing a thiazoline and d-Alanine.

In this Example, enzyme specificity rules were formalized to facilitate their algorithmic combination to create a peptide with a defined PTM pattern. Four leader-dependent enzymes (TgnB, PlpXY, PaaP and LynD) and five tailoring enzymes (PalS, ThcoK, PadeK, EpiD, LasF) were selected to represent diverse chemical modifications, species, and RiPP classes (Table 5) [18, 54, 59, 78-81]. Most have little prior information in the literature regarding substrate preferences. Escherichia coli was selected as the chassis because RiPP enzymes often work in this host and the “Marionette” strains allow the independent control of up to a dozen genes [82-84]. An N-terminal SUMO RiPP stabilization tag (RST; as described in Example 8) was used to increase the concentration of precursor peptide and simplify leader cleavage, which can be difficult to predict [85]. Mutagenesis strategies were developed to efficiently extract the enzyme rules: recognition site, distance constraint, and core tolerance (FIG. 10A). An automation pipeline that spans oligo synthesis to ESI-MS analysis was used to evaluate over 1000 precursor peptide variants. The substrate rules were put into a form that simplifies their combination, to computationally design a precursor peptide that is modified by multiple enzymes. As a proof-of-principle, heterocyclized peptides that contain a thiazoline, beta-amino acid, and phosphorylated serine were designed and it was verified that 4/5 had the correct modifications, whereas there was an estimated 1 in 10 million chance of success if designed randomly. This Example lays out a strategy to mine RiPP enzymes with the data necessary to inform retrosynthesis algorithms to aid the design of desired post-translational patterns.

### Results

### Characterization of Leader-Dependent Enzymes

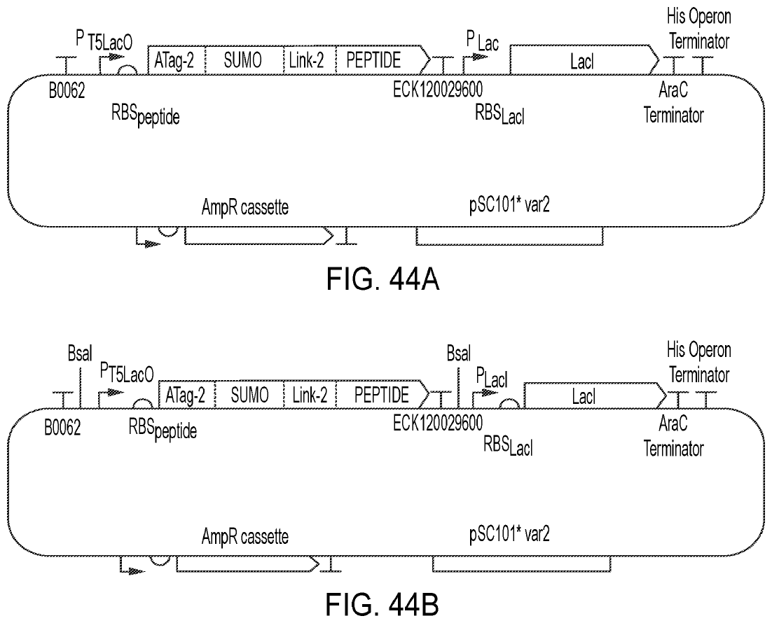

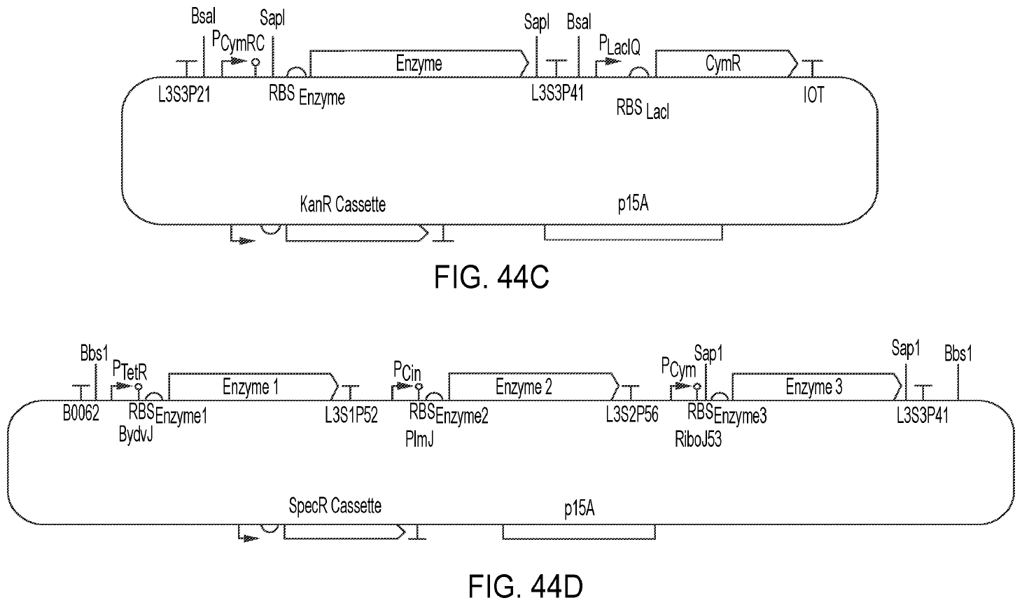

A microtiter-based peptide expression, purification, and analysis pipeline was adapted to study modification of many peptide mutants/variants by individual modifying enzymes. This is a two plasmid system, with modifying enzyme produced from a p15A medium-copy plasmid and precursor peptide expressed from a pSC101 origin mutated to maintain at medium copy number (var 2, [87], FIGS. 44A-44D). Modifying enzyme expression was controlled by the cumate-inducible CymR repressor with matching promoter due to the repressor's high expression and low leak, while precursor peptide expression was controlled by the IPTG-inducible lac repressor with the T5LacO promoter due to its high expression (leak was acceptable for precursor peptide expression) [84]. Peptide expression was stabilized with by the RST, which includes an N-terminal hexahistidine (HIS₆) tag for affinity purification, a SUMO stabilization tag, and a TEV cleavage site for liberation of the precursor peptide from SUMO (with residues “GC” remaining with the liberated peptide—G from the TEV cleavage site and C for compatibility with SAMDI analysis). Ribosome binding sites (RBS) were custom designed for each modifying enzyme using the RBS calculator to normalize expression levels, while a single RBS was used with the peptides since the RST sequence was consistently downstream and insulated the RBS from different precursor peptide sequences[88, 89]. A ribozyme was also used for modifying enzyme expression to stabilize mRNA and minimize effects of different promotors on translation (required for multi-enzyme modification) [90].

TABLE 5
Enzymes investigated in Example 3.

| RiPP Class | Enzyme | Enzyme | Peptide | Organism | Ref |
|---|---|---|---|---|---|
| microviridin | lactone cyclase | TgnB | TgnA* | Bacillus thuringiensis | 58 |
| pantocin | glu-glu cyclase | PaaA | PaaP | Pantoea agglomerans | 59, 78, 136 |
| spliceotide | tyrosine excisionase | PlpXY | PlpA2 | Pleurocapsa sp. | 18 |
| cyanobactin | thiazoline cyclase | LynD | TruE* | Prochloron spp. | 39 |
| lasso peptide | carboxylic acid methyl- | LasF | LasA | Lentzea kentuckyensis | 79, 113 |
| | transferase | | | | |
| glycocin | cysteine glycosyl-transferase | PalS | PalA | Aeribacillus pallidus | 110 |
| lanthipeptide | de-carboxylase | EpiD | EpiA | Staphylococcus epidermidis | 54, 106 |
| lasso peptide | serine kinase | ThcoK | ThcoA | Thermobacillus composti | 80, 81 |
| lasso peptide | serine kinase | PadeK | PadeA | Paenibacillus dendritiformis | 80, 81 |

*in this Example, truncated forms of the wild-type TgnA and TruE peptides were used, which only included one core sequence (see Table 8).

Nine RiPP modifying enzymes were selected for analysis in this Example (Table 5; see also Table 7). Four of the selected enzymes were leader dependent and needed recognition sites and spacing constraints elucidated. Three of those (PlpXY, LynD, and PaaA) contained the RiPP recognition element (RRE) domain previously shown to be responsible for leader binding [14, 15], while TgnB is an ATP-Grasp microviridin-class enzyme with a less-studied binding mechanism. These four enzymes are from different bacteria genera, catalyze diverse chemical modifications, and result in different physicochemical properties in the modified peptide:

(1) TgnB, from Bacillus thuringiensis, covalently links glutamate/aspartate residues with serine/threonine residues to form the bi-cyclic depsipeptide thuringeinin[58]. The resulting cyclic peptide is a potent antidigestive (digestive protease inhibitor) and is rigid and constrained, both properties of interest in the peptide drug-discovery community [6]. The enzyme was codon optimized and synthesized, and used to modify a truncated peptide substrate with only one core (versus the three-core repeat in the native TgnA peptide)[58].

(2) PlpXY, from Pleurocapsa sp. PCC 7319, excises tyramine (the amine, alpha carbon, and sidechain of tyrosine) by breaking the peptide backbone and re-fusing it, resulting in a ketone containing beta-amino acid [18]. The modification is interesting both in its chemical reactivity (it can be used as a click substrate), and its uniqueness—no other RiPP enzyme known alters the peptide backbone as extensively. The enzyme PlpX and its RiPP recognition element PlpY were both codon-optimized and expressed as a two-gene operon and used to modify PlpA2, one of three core peptides in the cluster.

(3) PaaA, an antibiotic from Pantoea agglomerans, performs a Claisen condensation between two adjacent glutamate residues, resulting in the fused-ring heterocycle indolizidine [78]. This alkaloid moiety is not typically associated with RiPP biosynthesis, but is prevalent in many bioactive small molecules [91]. The enzyme was codon optimized and was used to modify its native precursor peptide (also codon optimized).

(4) LynD, from Lyngbya sp., dehydrates a cysteine with a peptide backbone amide to form a five-membered heterocycle. The resulting heterocycle, thiazoline, spans what was the amide bond, creating a protease resistant backbone[92]. Thiazolines retain the planar structure of the amide [92] and can be oxidized to aromatic thiazoles by cyclodehydratases found in some RiPP clusters[93]. Due to their valuable properties, thiazol(in)e heterocycles are frequently found in bioactive natural products and approved drugs[92]. LynD was codon optimized, and was used to modify a single-core truncation of TruE, a precursor peptide from a homologous pathway.

To generate the peptide expression plasmids, and leader mutants thereof, some were ordered as oligos, PCR amplified, and cloned into TypeIIs expression vectors, but a majority were synthesized and assembled by Twist Biosciences. From Twist, peptide vectors were rehydrated and immediately co-transformed with their cognate modifying enzyme plasmid in microtiter 96-well plates. Because only clonal, sequence verified plasmids were used, co-transformants were directly selected for by growing in LB supplemented with kanamycin and carbenicillin, without plating on agar and picking colonies. After overnight incubation, stationary phase cultures were diluted 1:100 into expression media and maximally-induced at approximately mid-log to decrease potential toxicity effects on growth[94]. A high-velocity microtiter plate shaker was required due to the use of deep 96-well plates. It was found that shaking below 900 r.p.m. led to cell sedimentation and highly variable expression. The peptide/enzyme expressions were conducted in TB media, such that conditions for all enzymes were identical.

Liquid-chromatography coupled to mass spectrometry (LC-MS) was used for peptide analysis. SUMO-tagged peptides were analyzed directly (without tag removal) in order to decrease the number of processing steps and reduce peptide-to-peptide run variability (the tag buffers against the chromatographic properties and solubility of diverse peptides). Peptides purified and eluted via IMAC were directly injected on the LC-MS for analysis. Since all of the modifications studied in this Example resulted in a change in mass between the unmodified and modified peptide, extracted compound chromatograms could be generated based on the expected masses of the unmodified, partially modified (if relevant), and modified peptides. If a chromatogram contained a peak, it was fit with a skewed gaussian[96], and the resulting fit was used to calculate peak area. Peak areas for modified, partially modified (if applicable), and unmodified peptide were summed to calculate the total peptide observed, which was then used to calculate the fraction of each peptide modification state.

While this process was chosen due to its simplicity and scalability, it does have two limitations: 1) Modified, partially modified, and unmodified peptide masses were sometimes not fully resolved in the MS. For the tagged large peptides analyzed (15-25 kDa), the isotope distribution could span 15-25 Da. If the modification being studied caused a mass shift of <15-25 Da, the isotope distributions between the unmodified and modified peptides would not be fully resolved, leading to crossover during integration of the modified and unmodified peptides. Similarly, spurious sodium adducts could cause a 22 Da mass shift, resulting in overlap with enzyme-catalyzed 14 Da (LasF) and 18 Da (TgnB and LynD) mass shifts, also affecting integrations and fraction modified calculations. 2) Multiple charge states are required to reliably annotate a peptide as present, which raises the limit of detection. On the machine that was used, the SUMO-tagged peptide limit of detection was estimated to be a peak area of ˜10⁴⁻⁵. The median peak size observed was ˜10⁶, meaning that a peptide with a fraction modified of 0.0 could actually have been as high as 0.1, if the modified peptide intensity was just below the detection threshold, or fraction modified of 1.0 could have been as low as 0.9 (though this would have had no effect on intermediate values of fraction modified). Most of the overlapping isotope effects were solved by extracting ECCs using a small m/z window around the expected mass of each peptide, such that regions of isotope overlap were ignored. For any remaining effects of overlapped isotope distribution, as well as sodium adduct and high limit of detection effects, the effects should largely have been dependent on the modification mass shift and the peptide being studied. Therefore, the effects could be countered by only comparing fraction modified within the same modification, since the effects should be similar (and cancel out) for similar peptides with the same modification.

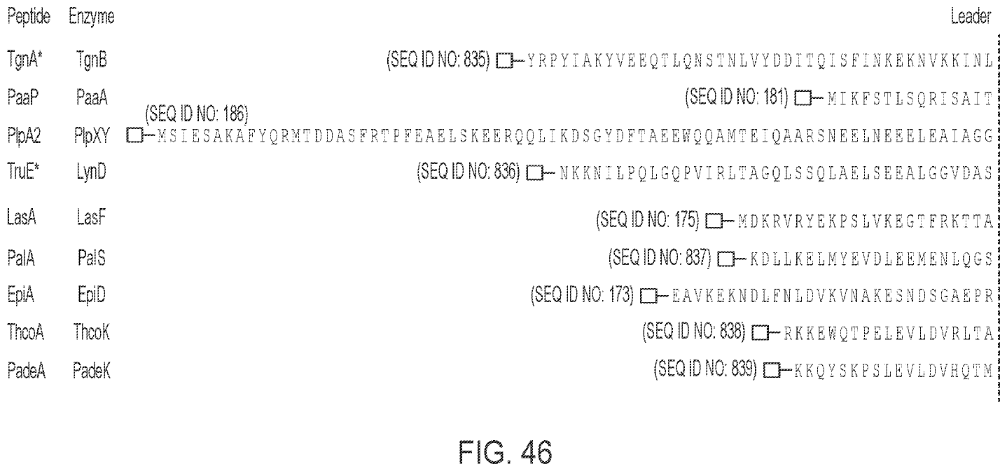

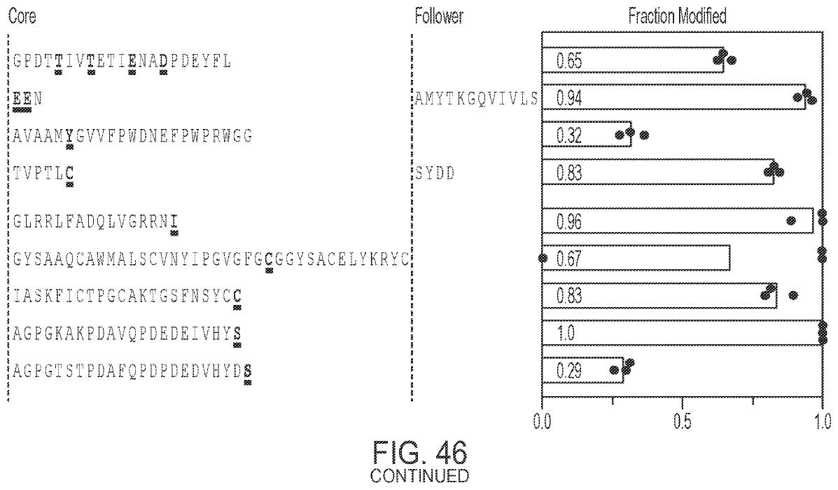

Using the outlined pipeline, the four leader-dependent modifying enzymes were used to assay for modification (FIG. 46). Fraction of peptide modified varied between the enzymes (0.32-0.94), qualitatively in agreement with data presented in previous publications[18, 39, 58, 78]. PaaA and LynD were the most efficient, with 94% and 83% of their peptide modified, respectively. TgnB was distributive[58], like other microviridins[97], meaning that the enzyme binds, forms a single lactone, unbinds, and the process repeats until all lactones are formed. Both lactones were formed in 65% of the peptide, with the remaining 35% evenly split between unmodified and a single lactone modification. The lowest fraction modified was observed for PlpXY, with 32% of PlpA2 modified, although this was similar to the low-turnover shown previously [18]. Encouragingly, the platform gave reproducible values, with standard deviations as low as 2% (LynD and TgnB) and not above 3.8% (PlpXY).

Identification of Recognition Sites within Leaders

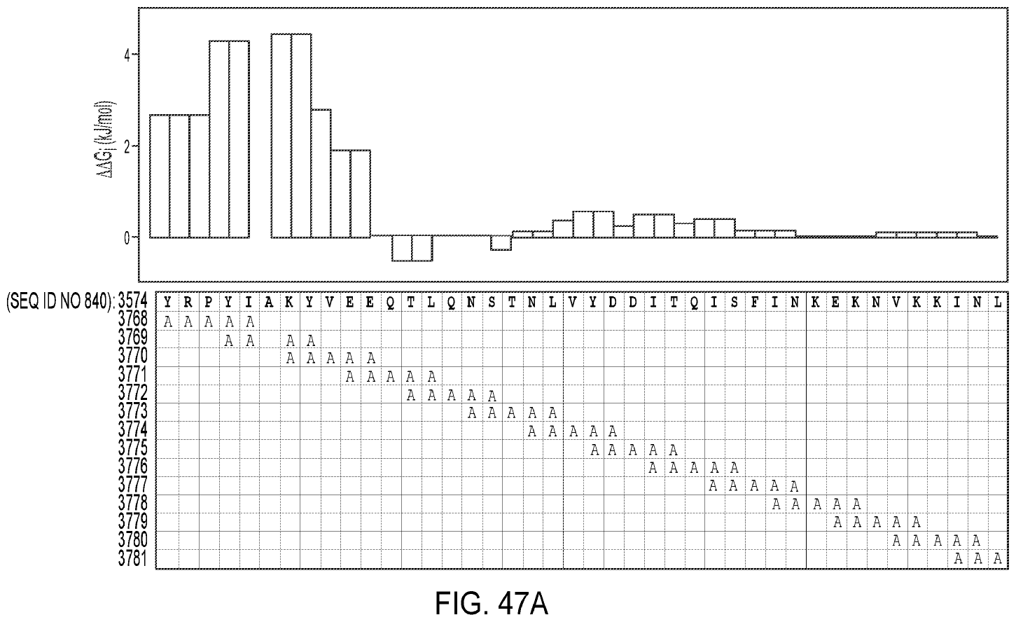

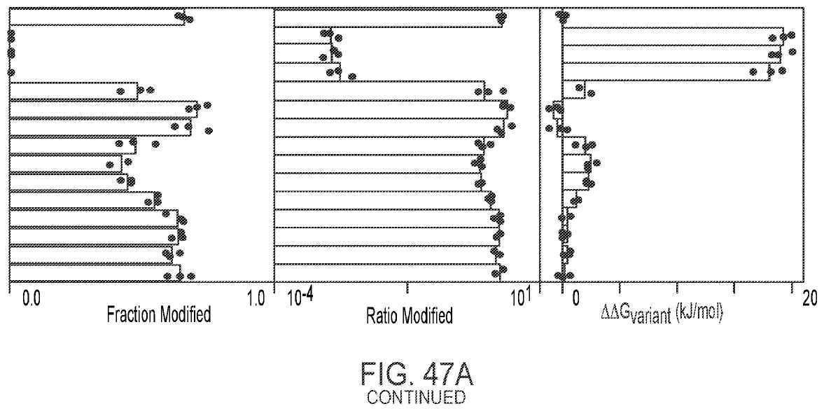

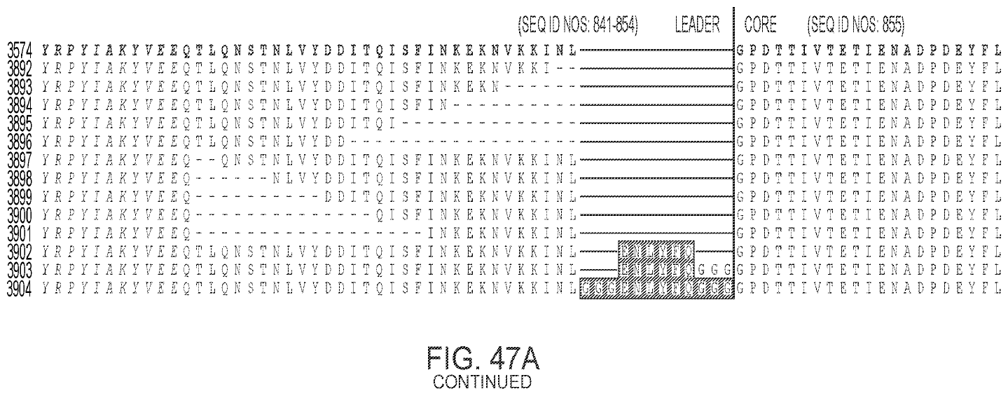

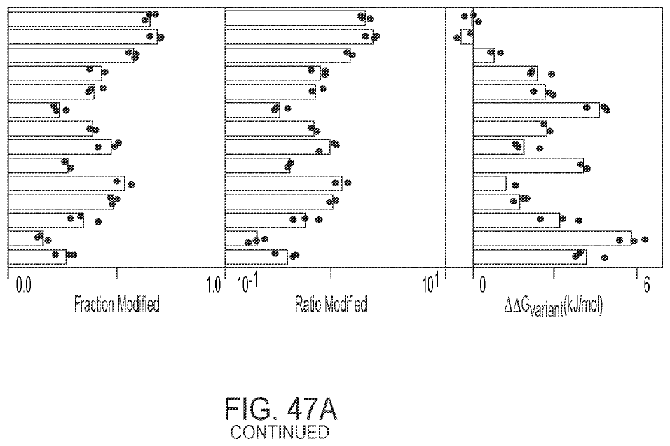

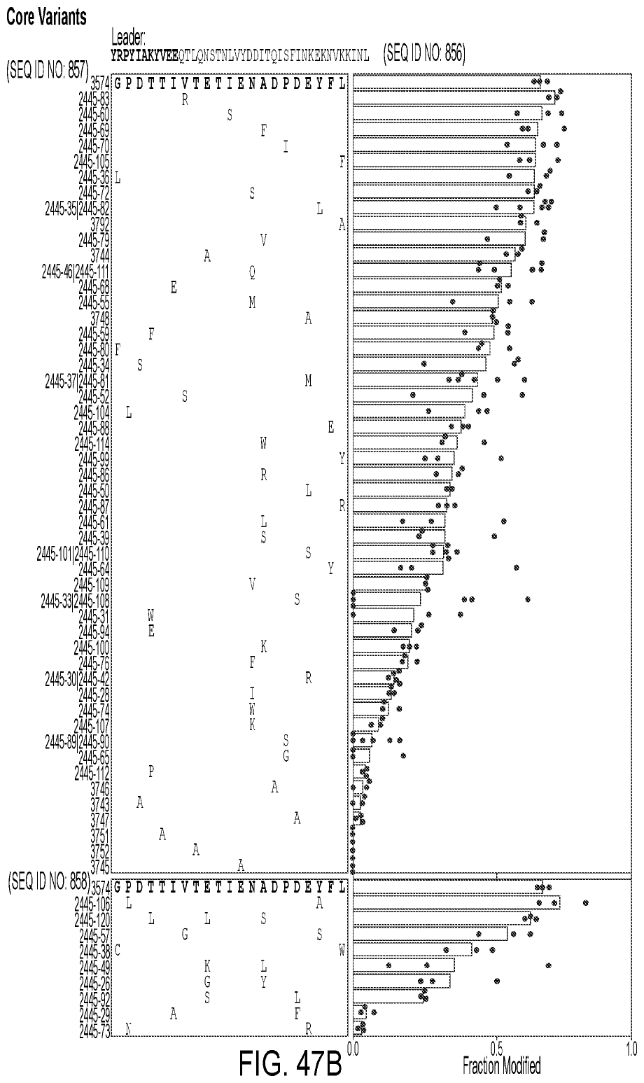

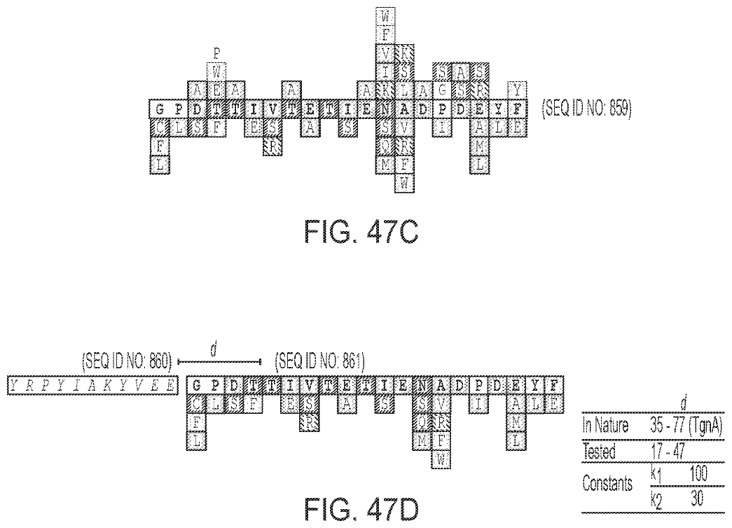

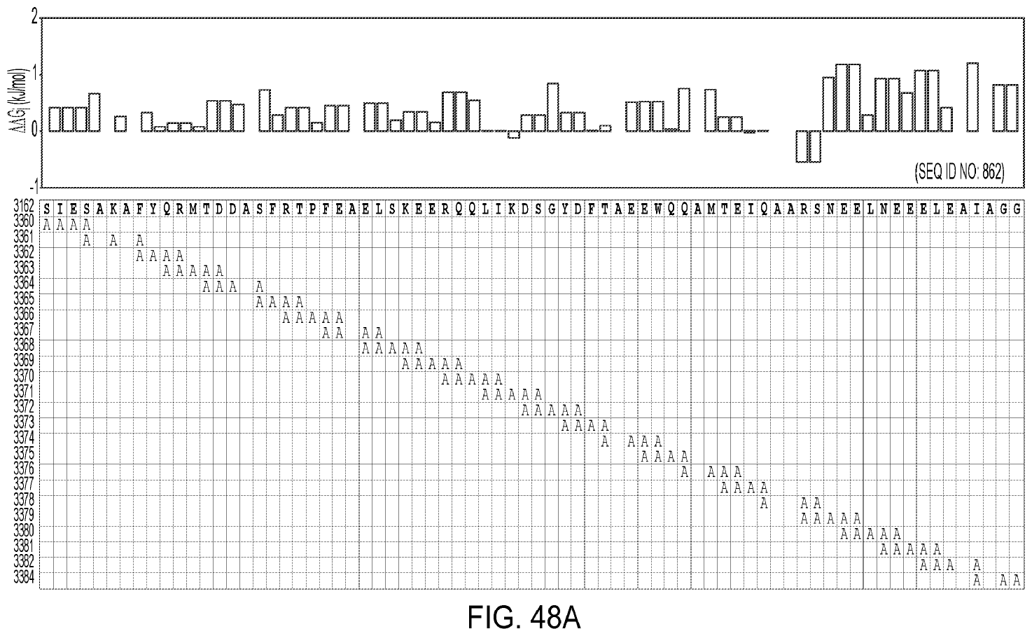

A simple approach was taken to deduce each enzyme's RS sequence(s). Alanine scanning is effective in finding the RSs, by measuring when the modification to the core is disrupted [60]. However, making a single substitution at every position is inefficient, particularly for long leaders and provides unnecessary resolution given that the smallest RS known is 7 amino acids[98] (excluding protease sites). Instead, blocks of 4-5 alanines were used to scan the leader and measure the impact on the fraction modified (block size dependent on leader length). The block was iteratively moved by 2-3 residues for each mutant (FIG. 10B). As an example, only 14 mutants of the 42-residue TgnA leader needed to be made to identify the RS for TgnB (FIG. 10C). When the alanine block disrupts the RS, the efficiency of modification drops dramatically, in this case at the far N-terminus of the leader. The results of these experiments for the leader-dependent enzymes are shown FIGS. 47A-47D, 48A-48D, 49A-49C, and 50A-50D.

A thermodynamic model was derived to infer the per-residue contribution to the binding of the modifying enzyme. This was simplified by assuming that the reaction follows Michaelis-Menten kinetics, where reversible binding to the leader precedes modification and release. This treats the binding and unbinding as being at quasi-steady state with respect to the production and degradation of the peptide; in other words, the ratio modified ρ, is the equilibrium value. Then, the change in the free energy of binding of the variant n with respect to the wild-type is

$$ΔΔ {G}_{n} = Δ {G}_{n} - Δ {G}_{wt} = - RT ln ( \frac{{ρ}_{π}}{{ρ}_{wt}} ) \qquad ( Equation 1 )$$

where R is the gas constant and T is temperature. If the contribution of each residue i of a mutant contributes additively to the free energy change, then

ΔΔGₙ=Σᵢ₌₁^{M}ΔΔGᵢ (Equation 2)

where M is the number of mutated residues. An algorithm was developed to assign ΔΔGᵢ values using all of the variant data. Initially, the contribution of ΔΔGₙ was divided equally amongst the mutated residues (for example, divided by 5 for a 5-alanine block in which none of the wild-type residues replaced by the block were originally alanines). However, some residues were mutated in two variants, so the residue was assigned a ΔΔGᵢ value of the mean of the two ΔΔGₙ/M values. The resulting ΔΔGᵢ assignments violated equation 2 (ΔΔGᵢ values will not sum to ΔΔGₙ within a variant), so ΔΔGᵢ values were adjusted iteratively and in small increments (similar to a force-directed graph) until the constraint of equation 2 was satisfied for all variants.

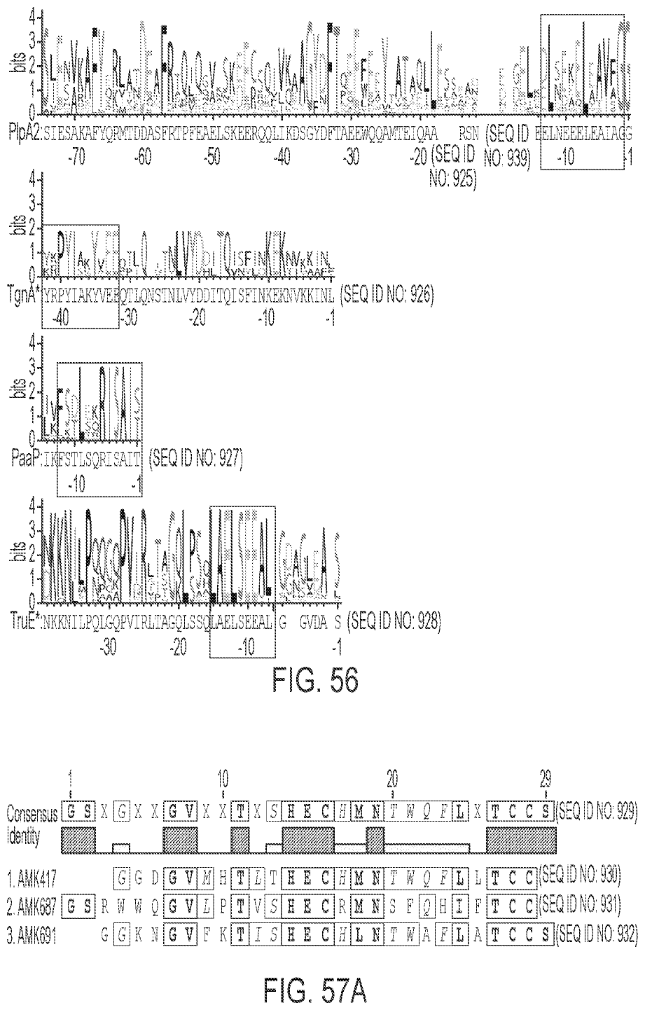

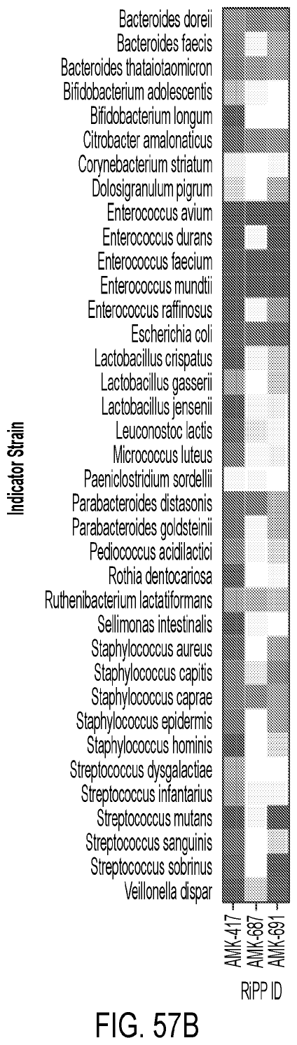

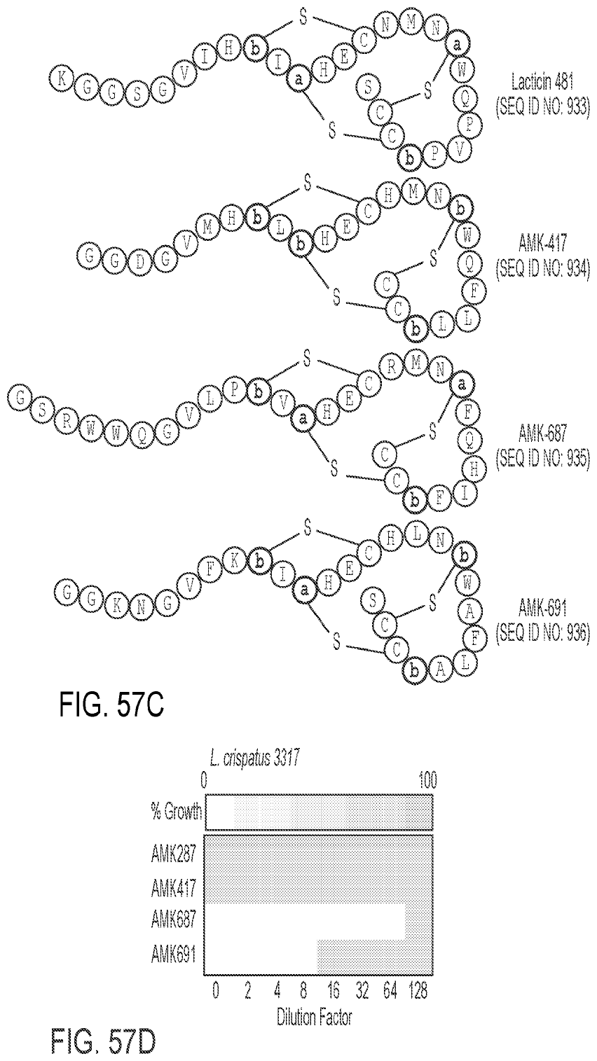

The result of this calculation is shown in FIG. 10C for TgnA/TgnB. Eleven residues at the N-terminal end were determined to have high ΔΔGᵢ values. This was in agreement with previously published observations that deletion of residues −42 through −35 or −34 through −29 residues at the N-terminus of the TgnA leader peptide knocks out TgnB modification while deletions in other sections of the leader are tolerated [58], as well as high conservation of residues −40 through −33 in TgnA homologs (FIG. 56). These data were mapped to the leader sequence in FIG. 10D, shaded according to the magnitude of ΔΔGᵢ. Because ΔΔGᵢ values were based on alanine-block replacements, they may not accurately depict the edge of an RS. The RS defined for the specificity rule is outlined by a box in FIG. 10D. For several enzymes, it did not exactly correspond to the regions of high ΔΔGᵢ because additional information was incorporated into the designation; either expanding it to be conservative or shrinking it if there was information that the residues were not important.

One source of additional information was leader structure. A Deep Convolutional Neural Field algorithm (RaptorX Structure Property Prediction) was used to predict the secondary structure of the leaders (FIG. 10D) [99]. Of the four peptides, TgnA was the only leader RS that did not align with an alpha-helical region, which was surprising given that the TgnB homolog MdnC recognizes an alpha-helix in the MdnA leader[98]. The sequence itself is also similar, with TgnB recognizing “YRPYIAKYVEE (SEQ ID NO: 108)”, with bolded residues aligning closely with the “PFFARFL (SEQ ID NO: 109)” recognition site highly conserved in microviridin leader peptides[98]. Analysis of the MdnA peptide with RaptorX predicted an alpha-helix at the RS, indicating that the TgnA sequence may elude secondary structure prediction by this algorithm or the TgnB enzyme does not bind a helix like MdnC. Closer investigation of the secondary structure prediction from RaptorX showed that the recognition site is ˜10 times more likely to have a helix than anywhere else in the leader peptide, but ˜3 times less likely than beta-sheet or coil at those positions. Importantly, this showed that secondary structure alone cannot reliably predict an RS. PlpXY, PaaA, and LynD all bind to the RS via a RiPP recognition element (RRE) which has been shown to bind to alpha-helical peptides [14]. Indeed, the high ΔΔGᵢ residues corresponded to regions predicted to adopt a helical structure. In the case of LynD, even though only three residues were calculated to have a high ΔΔGᵢ, the boundaries of the RS were extended to encompass more of the helix (FIG. 10D). In contrast, the final glycine was removed from the PlpXY recognition site in PlpA2 because it was not part of the helix, and the “GG” leader motif is commonly necessary for cleavage between the leader and core, not for modifying enzyme recognition [100].

Sequence conservation within peptide homologs was also incorporated. Encouragingly, for all of the leader peptides, regions of high ΔΔGᵢ values corresponded to regions of high conservation in weblogos of peptide homologs (FIG. 56). Similar to structural predictions, sequence homology was used to inform the boundaries of the recognition sites. The LynD recognition site did not include the full helix (“SQ” at the beginning is not included) because those positions had poor conservation in TruE homologs (FIG. 56). The resulting LynD recognition site, “LAELSEEAL (SEQ ID NO: 110)” is highly conserved in other cyanobactin peptides [39], and has been shown to be sufficient for modification with LynD homologs[76]. While the first two residues of the TgnB leader were kept in the recognition site, lower conservation at those positions may indicate that they are not necessary. Finally, the two positions N-terminal to the PlpA2 RS (“NE”) were not included because they are not conserved in PlpA2 homologs.

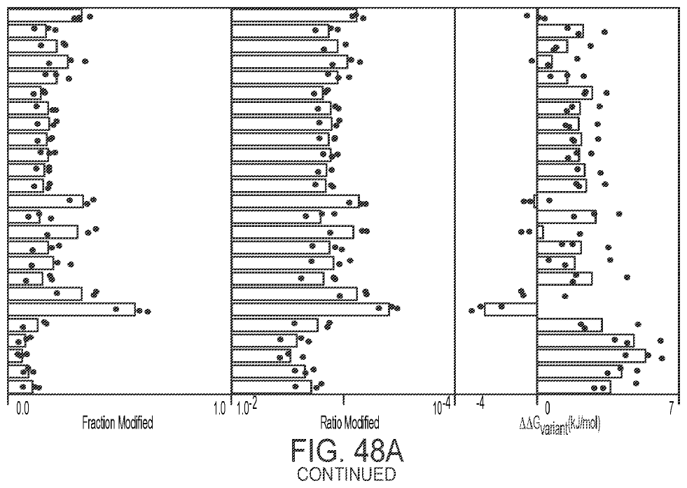

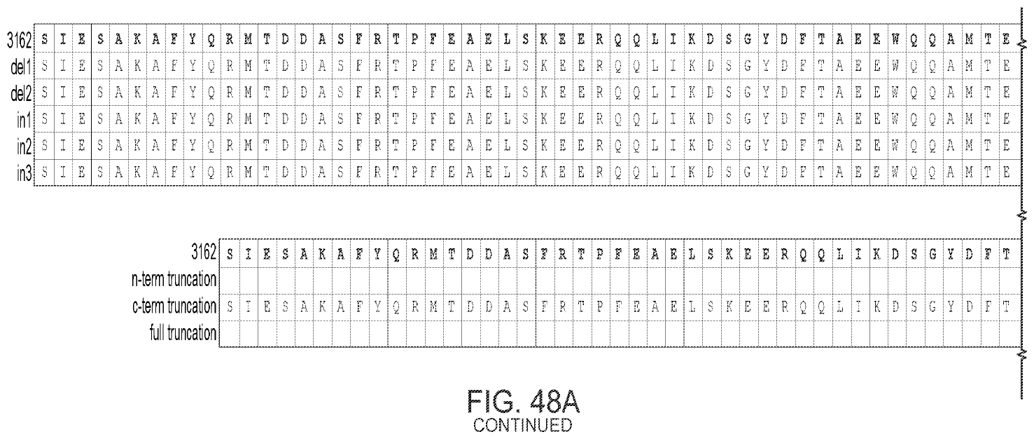

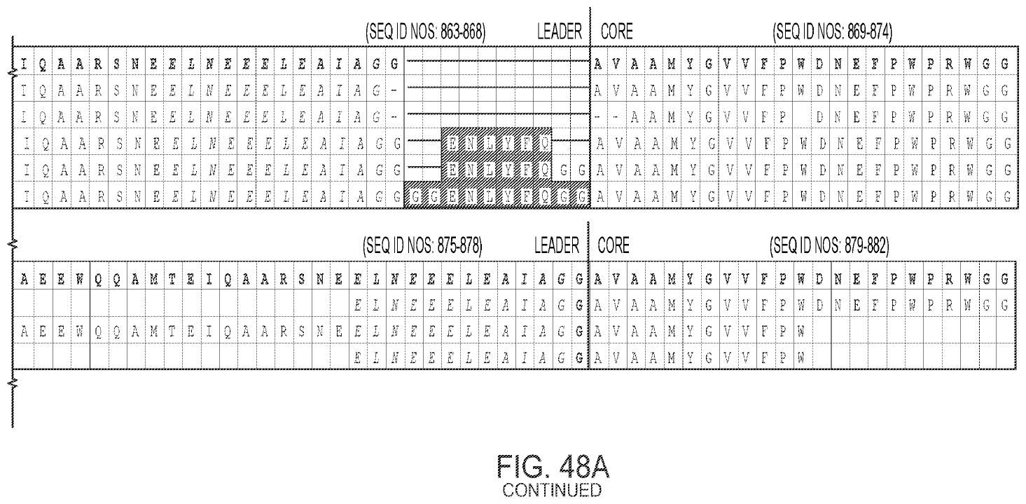

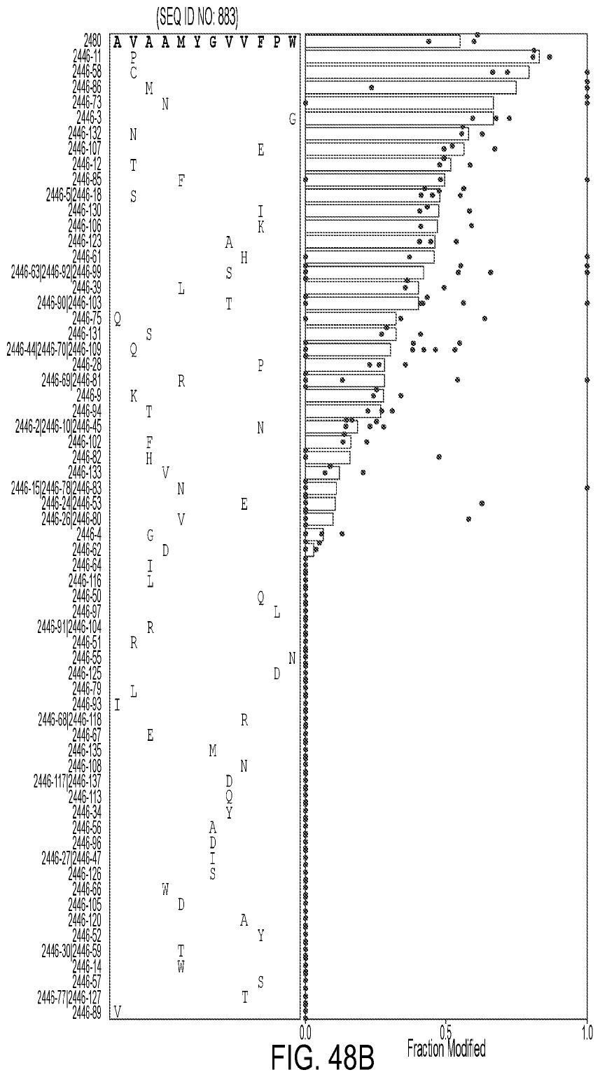

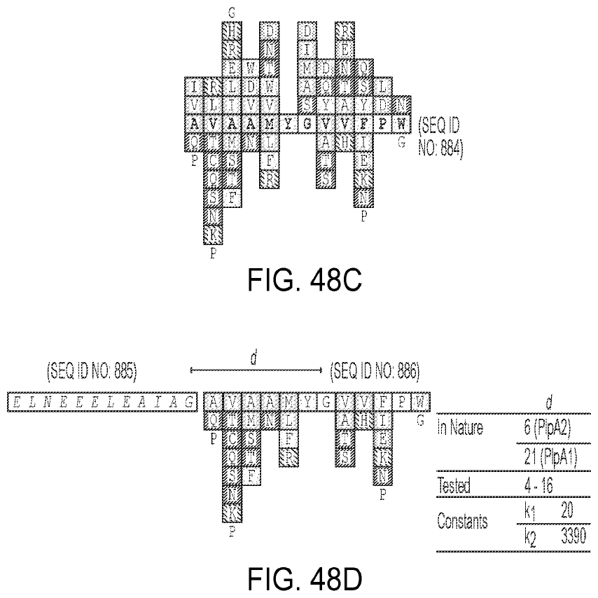

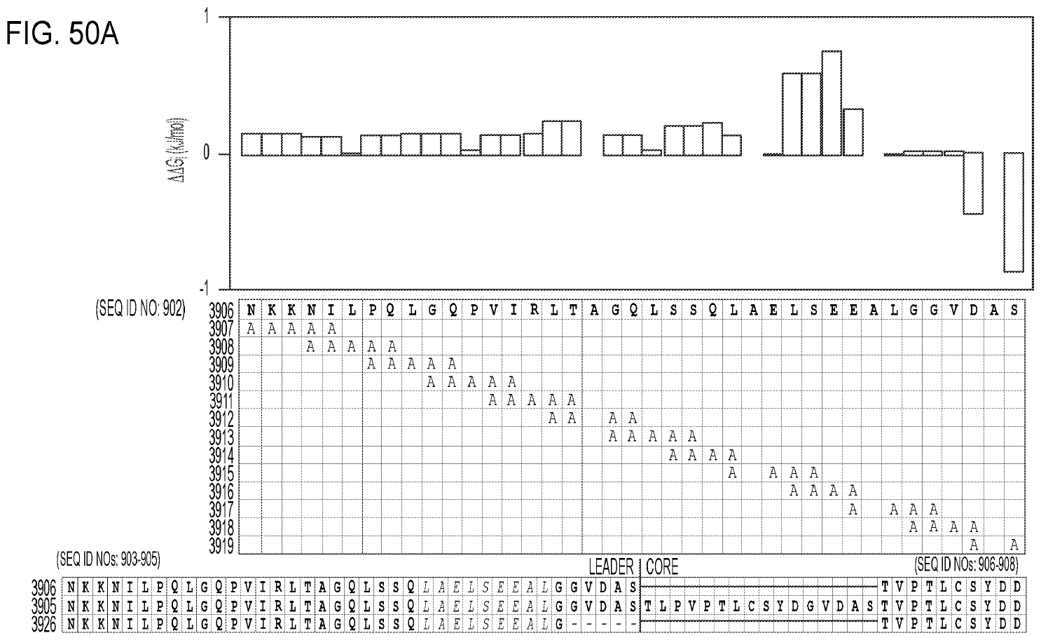

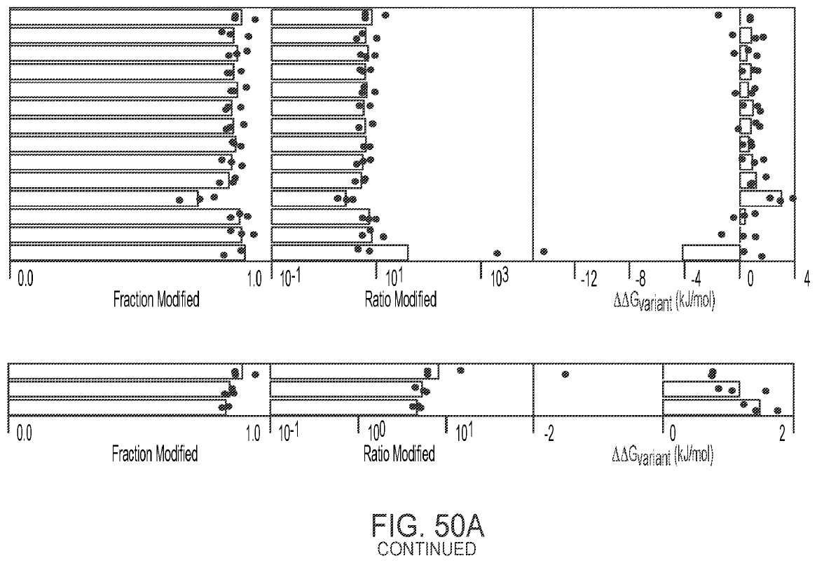

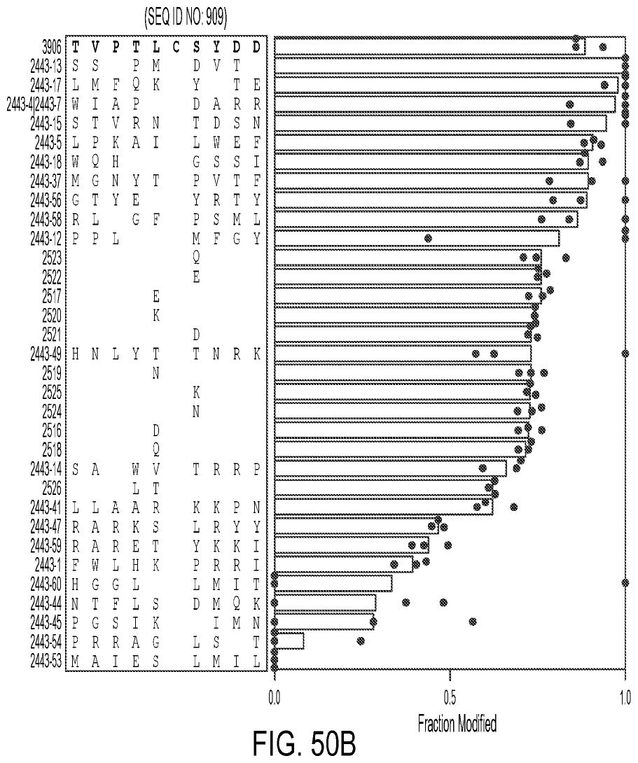

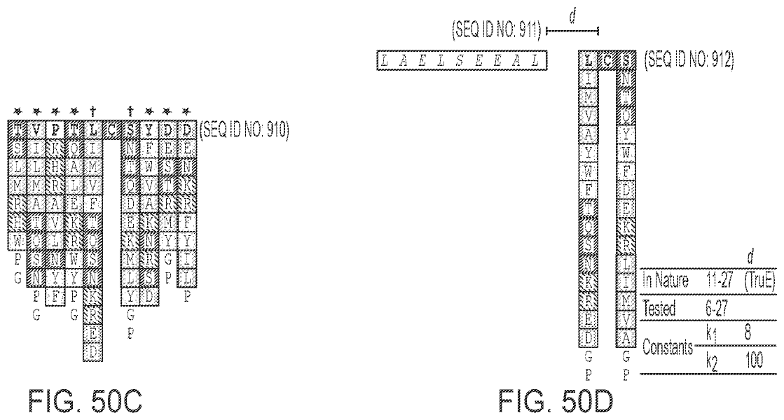

While the alanine scans showed that sequences in the RSs are necessary, and homologous sequences and structural predictions can help validate those data and inform boundaries, they did not prove that the RS is sufficient for modification. For each of the peptides, truncations were tested to remove sequence that should be unnecessary. The TgnA RS is at the N-terminus of the leader, so only truncations between the RS and the core were possible. The effect of truncations on RS-to-modification spacing versus sequence importance could not be differentiated, but truncations of various sizes were generally tolerated. Most truncations were modified over half as well as wild-type, and were modified as well as or better than similarly-sized insertions, indicating that the modifying enzyme is sensitive to changes in RS-modification site spacing. Previously reported deletions scanned through the TgnA leader also agreed with annotation of the TgnB RS as necessary and sufficient for modification, where only deletions that included RS residues were unmodified [58]. Both the TruE and PlpA2 peptides included sequences N-terminal to the RS, removal of which was well-tolerated by each respective modifying enzyme, with fraction modified similar to that of full-length leader (FIG. 48A and FIG. 50A). Removal of residues between the LynD RS and the core was also tolerated by LynD (FIG. 50A). The PaaP RS consisted of nearly the entire leader, so leader truncations were not tested, but truncations to the follower peptide were tested to determine if it is necessary for modification. Previous work has shown that truncation or removal of the follower peptide is not tolerated by PaaA [78], but that the follower sequence can be mutated without breaking modification [59]. Similarly, removal of only three amino acids from the C-terminus of the follower was observed to decrease modification from 89% to 39%, with removal of nine amino acids breaking modification. While the sequence is important for modification, scanning site saturation mutagenesis of the entire peptide showed that the sequence in the follower is mutable, unlike residues in the RS of the leader[59]. Based on this, the follower was treated as an extension of the core peptide, rather than as a “structural” element of the peptide. The sequence constraints in the follower were therefore elucidated later as part of the core sequence motif.

The final recognition site sequences are outlined in boxes in FIG. 10D and are listed in Table 5. The sites were similarly sized, ranging from 9-12 amino acids, but varied in their placement in the leader, ranging from N-terminal (TgnB) to C-terminal (PaaA and PlpXY) and between (LynD). The sites contained large numbers of hydrophobic amino acids (L/I/A/F/V/P), in agreement with observations that hydrophobic interactions are a contributor to affinity between modifying enzymes and RSs [14] and protein-protein interactions as a whole [101]. They differed in the charged residues present, with LynD and PlpXY containing negatively charged glutamate residues and PaaA and TgnB containing a single arginine and lysine, respectively. Given the helicity of the RSs (all but TgnB), charged residues may be solvent exposed (opposite the binding face), or participate in salt-bridges as part of the interaction. The annotated RSs also agreed with previously published work on these enzymes, when available. Deletion of amino acids in the N-terminus of TgnA precluded modification by TgnB [58], in agreement with annotation of the RS at the N-terminus of the leader. The four leader residues previously shown to be important for modification of PaaP by PaaA [59] were all included within the annotated RS. The LynD RS, as annotated, has previously been used both in vivo and in vitro with LynD and homologs of LynD [39, 42, 102]. This is the first known description of the PlpXY RS in PlpA2.

### Determination of RS Spacing Constraints

Variants were designed to alter the spacing d between the RS and the modified residue. An alternative would be to define d as the distance to the start of the core sequence, which could be more intuitive for enzymes that modify multiple core amino acids, such as TgnB [58]. However, the distance to the modification was selected as it was more likely to be the physical distance to the modification site itself that influences modification rather than the distance to the core/leader cleavage site. Additionally, during forward engineering of precursor peptides, it functions as a constraint on core length by keeping modifications from being allowed at infinite core positions away from the leader. As such, d was defined as the number of residues between the RS and the modified amino acid. If multiple amino acids were modified (for example the two lactone cycles in TgnB modification), it was the distance to the first modified amino acid.

Changing d from its optimal value was expected to lead to lower modification efficiencies. In its simplest form, this can be treated as an energy well, where a wider well corresponds to more core positions being modifiable if RS position in the leader is kept constant. In contrast, a steep well indicates that the modification can only occur at a single residue, optimally spaced from the RS. A spring model is the simplest way to model this effect, which has been applied to similar biophysical phenomena, such as modeling the impact on ribosome binding that results from different spacing between the Shine-Delgarno and ATG start sites [88]. Using a spring model, RS-to-modification distances less than optimal would be “stretched” for modification, while distances greater than optimal would be “compressed”. The following equation can be derived from Hooke's Law,

$$ΔΔ {G}_{n} = \frac{1}{2} [ {κ}_{s} H ( d - {d}_{0} ) {( d - {d}_{0} )}^{2} + {κ}_{c} ( 1 - H ( d - {d}_{0} ) ) {( d - {d}_{0} )}^{2} ] \qquad ( Equation 3 )$$

where d₀ is the optimum spacing, κₛ and κ_{c} are the stretching and compression spring constants, and H(x) is a step function. Equation 3 could be changed to reflect other functions; for example, it might take on the form of a steep step function if there is a distance at which suddenly an enzyme is no longer active. It also does not have to be monotonic, with more complex forms modeling enzymes that exhibit multiple local minima or periodic behavior. In its current form, the stretching and compression constants define the width of the energy well described above, with small values of κ corresponding to a wide energy well with high spacing tolerance and large values corresponding to a narrow energy well with low spacing tolerance.

Leader variants were designed for each modifying enzyme to perturb the RS spacing, starting with TgnB. TgnA* has 35 residues between the RS and the first modified residue, with 31 of those being in the leader. Five truncation variants were designed by removing residues at the C-terminus of the leader, starting with two amino acids and increasing in increments of four amino acids to the longest truncation of 18 amino acids, representing over half of the spacer. Three insertion variants were also designed using a TEV cleavage site (amino acid sequence ENLYFQ (SEQ ID NO: 111)) and glycines as a spacer: the TEV site alone is a 6 amino acid insertion, TEV site followed by triple-glycine is +9 amino acids, and TEV site flanked by triple-glycines is +12. Each of these 8 variants was assayed for modification, and the fraction modified for the variants is shown in FIG. 10E. Values were converted to ΔΔG. (using equation 1) for each variant and plotted against the RS-modification distance. With the exception of the longest insertion variant, increased spacing deviations from optimal corresponded with decreased modification. The trend was fit with equation 3 to calculate the stretching and compression spring constants (Table 6) as 30 and 100 J·mol⁻¹·AA⁻², respectively, where do is set to the wild-type distance. These values implied that the enzyme was more tolerant of shorter spacer distances than longer, surprising given that the wild-type TgnA peptide contains a leader with three cores in tandem [58], with leader to modification distances of 35, 56, and 77. With a compression constant of 100 J·mol⁻¹·AA⁻², the farthest modification was predicted to have a ΔΔGₙ of 176.6 kJ/mol, effectively unmodified. While the data collected was used to fit the spring constants, this data indicated that a more complicated model, including variations of equation 3 with periodic behavior, may be necessary for distributive/multi-core enzymes like TgnB [103]. It is worth noting that modification of the full TgnA peptide using this expression platform was not observed[104], so it is also possible that the TgnA* spacing parameters described here are specific to the single core TgnA* peptide expressed as a SUMO fusion.

TABLE 6
RS spacing constraints
Parameter^{a}

| Enzyme | d₀ | κ₁ | κ₂ |
|---|---|---|---|
| TgnB | 37 | 100 | 30 |
| PlpXY | 6 | 20 | 3390 |
| PaaA | 0 | 40000^{b} | 40000^{b} |
| LynD | 11 | 8 | 100 |

^{a}Parameters for Equation 3.
^{b}No indel tolerated; Fit for ΔΔGₙ = 20 at d-d₀ = 1

PlpXY is known to be tolerant to varying core positions, since there are two precursor peptides associated with the cluster that have RS to modification distances of 6 (PlpA2) and 21 (PlpA1). The leader peptide (and RS sequence) of PlpA1 differs from PlpA2, so modification of the two was not directly compared, since modification differences due to distance cannot be separated from RS sequence differences. Instead, spacing parameters were elucidated similarly to TgnA*, using engineered insertion/deletion variants of PlpA2. Since the RS is one residue away from the C-terminus of the leader peptide and the modified tyrosine is also close to the N-terminus of the core, only three deletion variants were tested: deletion of the final glycine (−1), the final glycine and first two residues of the core (−3), and the final glycine and first four residues of the core (−5). The same insertion variants were tested as for TgnA*/B: insertion of a TEV cleavage site (+6), TEV cleavage site followed by a triple-glycine (+9), and TEV cleavage site flanked by triple-glycines (+12). The variants were assayed for modification, with variant effect on modification converted to ΔΔGₙ and fit with spring constants (FIG. 10D and Table 6). As expected, increases to RS-to-modification distance were well tolerated, with variants having near-wild-type modification, in agreement with the large spacing observed in PlpA1.

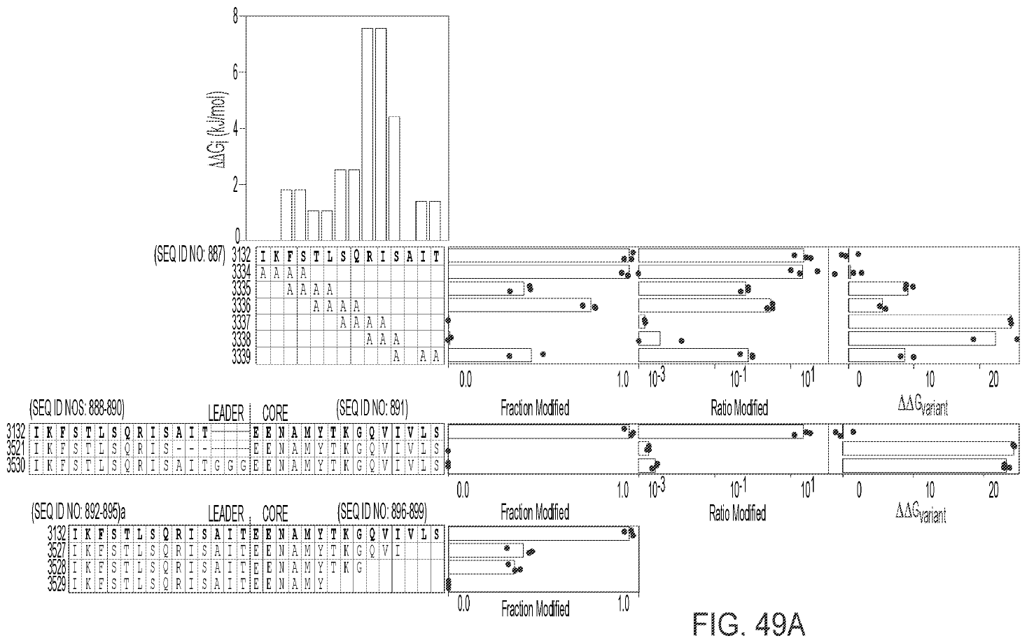

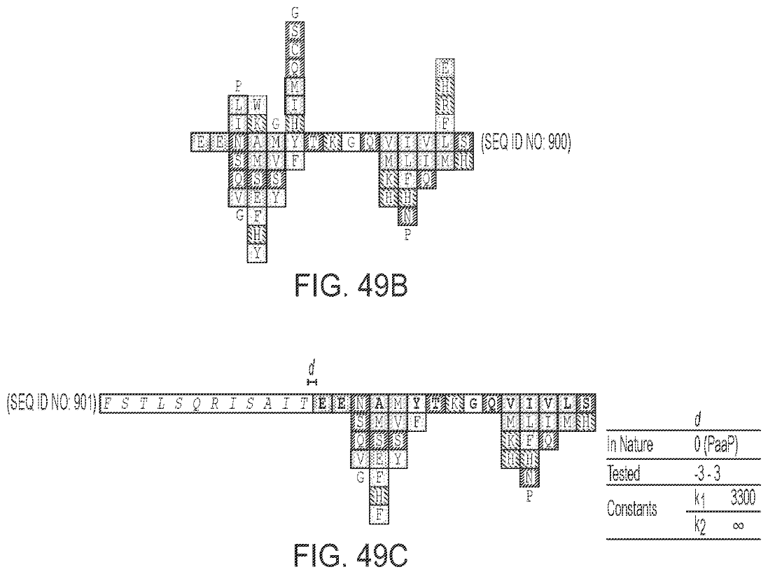

The PaaA RS has very rigid placement restrictions (FIG. 49A). The RS in the leader directly abuts the modified residues in the core, making it impossible to delete amino acids. Deletions that cut into the defined RS were found to abolish modification, while adding a small GGG spacer between the RS and the modification was also not tolerated. Therefore, a ΔΔGₙ of 20 was assigned for d values −1 and +1 from d₀, and solved for both κ constants using those values, with ΔΔGₙ=0 at d₀.

LynD, and homologous cyanobactin heterocyclases, are known to be tolerant to spacing changes in the precursor peptide [39, 42]. In nature, it modifies the LynE peptide, which includes the same “LAELSEEAL (SEQ ID NO: 110)” RS defined in the truncated TruE* peptide, with three tandem cores and modified cystines spaced 9, 12, 24, 27, 39, and 42 amino acids from the RS [39]. In the full-length TruE peptide, which was modified with LynD in this Example, LynD modifies cysteines in two tandem cores, with RS-to-modification distances of 6 and 27 amino acids (FIG. 50A). Modification of the full-length TruE peptide was compared with modification of the truncated TruE* peptide to identify a compression spring constant of 8 J·mol⁻¹·AA⁻². A single deletion variant, with five of the six leader residues between the RS and core removed, was fit with a stretching spring constant of 100 J·mol⁻¹·AA⁻² and tested.

### Tolerance to Core Mutations

Libraries varying the core of each RiPP were made to determine modifying enzyme tolerance to different amino acids. In general, the approach of using scanning site saturation mutagenesis (SSSM) was followed and applied to positions surrounding the modified residue(s) [56, 59]. Degenerate oligonucleotides, with codons replaced by NNK mixed bases, were used to build libraries and isolate core sequence variants. Typically, a single residue would be varied at a time, with all single-residue NNK libraries pooled together such that an individual library member has a random amino acid at a single random position (also known as a saturation mutagenesis single variant library or single codon randomization library, abbreviated as sSSSM for single SSSM). The pooled oligonucleotide libraries were cloned and individual variants were isolated and sequence verified. To increase coverage at each position, the number of core positions in the libraries was decreased and included only those surrounding and necessary for the modification. For cores with long C-terminal “tails” after the modification, truncations were made to the peptide's C-terminus to determine the minimal sequence necessary for modification. All four modifications were close to the N-terminus of the core, so the entire core N-terminal to the modification was always included in the libraries. PaaA and TgnB modifications used wild-type leaders for modification, while leaders with long N-terminal regions before the RS (TruE* and PlpA2) used N-terminal leader truncations shown to be sufficient for modification during leader/RS characterization (FIGS. 48A-48B and 50A-50B). The core libraries were cloned into the same expression system described above and used with identical growth/expression conditions.

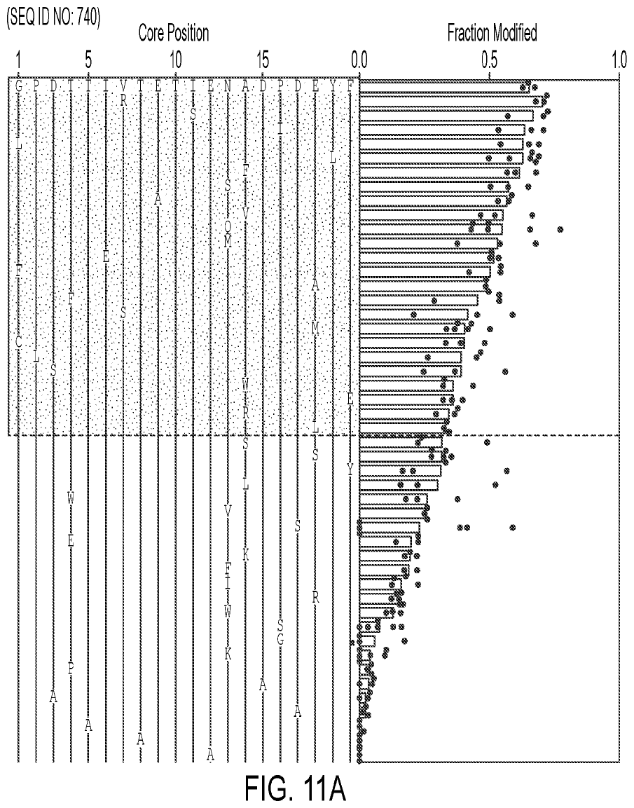

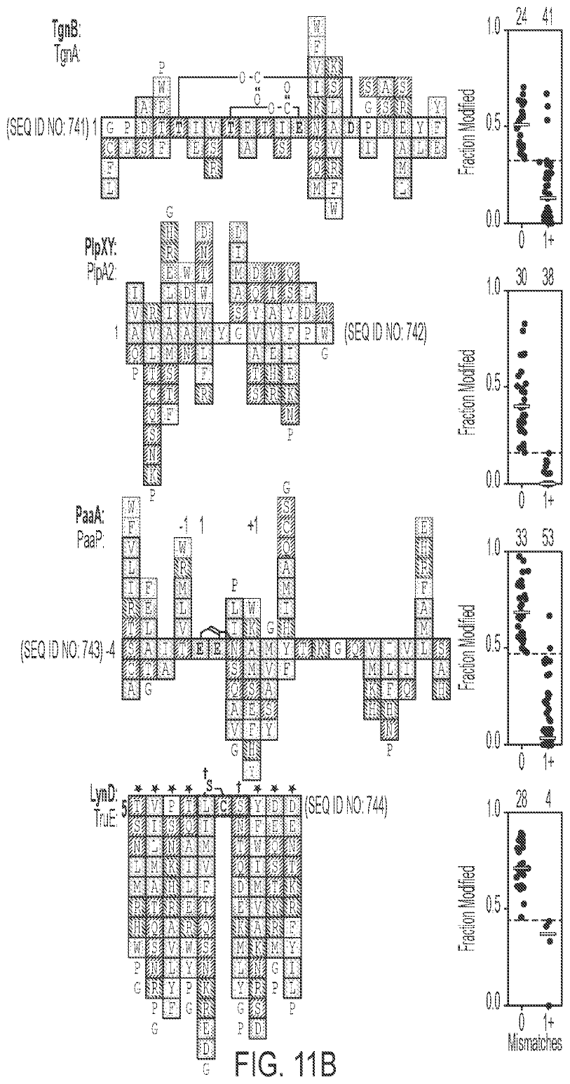

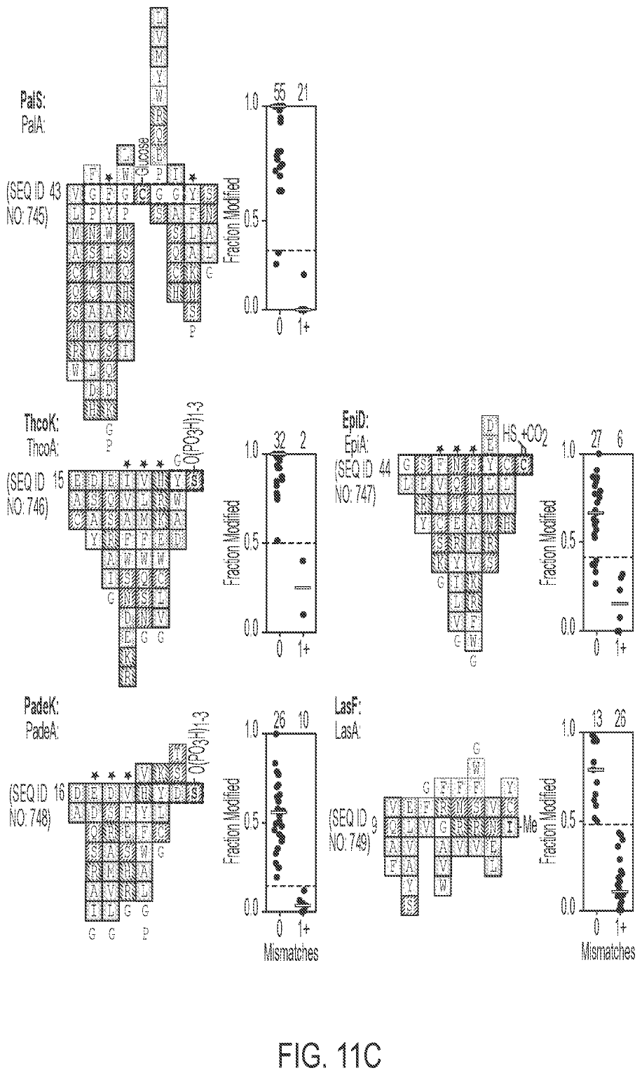

The raw data for the TgnA* core library are shown in FIG. 11A. For this library, the entire core sequence of 21 amino acids were included and 48 single-mutant variants were generated and analyzed. The library was composed of 21 oligonucleotides, each with a different core codon replaced by NNK, pooled together and assembled with the leader peptide into the peptide expression plasmid. The resulting sSSSM library was then co-transformed with the TgnB expression plasmid, and plated on agar such that each colony contained a unique peptide variant along with the modifying enzyme. Individual colonies were picked and peptide sequence verified before assaying for modification. As can be seen in FIG. 11A, the variants span all levels of modification. A criterion of 50% of the wild-type activity was set to consider an amino acid as being accepted. This conservative threshold was selected because, assuming additive effects, the multiple mutations that would arise from de novo peptide design would rapidly decrease the fraction modified. Of the variants tested, 25, or 52%, were modified above this threshold (FIG. 47B). Accepted amino acids at each position were then compiled into a core summary motif, shown in FIG. 47C, where accepted amino acids appear below the wild-type sequence and unaccepted appear above the sequence. Finally, amino acids that were observed unallowed/allowed at each position were compared with the other two core repeats present in the TgnA peptide (only one core repeat was used in TgnA*). Only a single amino acid of overlap was present between observed amino acid variants and the other natural cores—core position 20 is a tyrosine in the other cores. Though tyrosine was originally disallowed at position 20 since its fraction modified was slightly below the cutoff, the motif was updated to include it based on its presence in the natural core. Based on the same principle, the final core motif was updated to include all of the amino acids present in the other wild-type cores, and used to generate the motif shown in FIG. 11B.

Although the TgnA* library was designed to generate single-mutant variants, several variants were isolated with two mutations and one with three, which provided an opportunity to investigate mutation additivity (FIGS. 47A-47D). Mutations are additive when the free energy change of a double mutant is the sum of the individual mutations, ΔΔG₁₂=ΔΔG₁+ΔΔG₂. This is equivalent to multiplying the modified ratio from individual mutations for the double mutant. Two instances of non-additivity were found, both of which showed the compensatory recovery of a bad mutation. For example, in TgnA, the A14S mutation decreased the activity, but this could be compensated when both E9L and T4L were present, returning the triple mutant to wild-type activity. Similarly, P2L could be recovered by adding Y19A. Non-additivity was not observed for any of the other leader-dependent enzymes.

For PlpA2 modification by PlpXY, truncations to the C-terminus were first investigated to identify residues necessary for modification. Increments of three amino acids were removed from the C-terminus of the peptide until modification broke. Removal of 12 amino acids was tolerated, with fraction modified within error of modification of the wild-type peptide, while removal of 15 amino acids was not modified at all. This was in agreement with previous work which showed that the proline at position 11 was necessary for modification [18]. Based on this data, a library was built to include positions 1-12 of the core peptide. A similar sSSSM library was built as described for TgnA*, with 41 single-mutation variants isolated and assayed. In contrast to TgnA*, only half of the variants were tolerated, with one variant removed because of high variance amongst replicates (FIG. 48B). From this data, G7, V9, and P11 were observed to be restricted positions, with all tested mutations at those positions showing no activity (FIG. 48B). A core motif was built for PlpA2, shown in FIG. 11B. The observations were similar to those of Morinaka, et al[18]. They observed G7 to be essential, with replacement by an alanine not tolerated. At the methionine at position 5, mutations with 30 L, V, W, D, and T were tested, with L well tolerated, V poorly tolerated, and no tolerance for W, D, or T. Morinaka, et al annotated L and V as tolerated at that position, and F and E as not (similar to W and D, respectively)[18].

Based both on the six cysteines modified by LynD in the native LynE substrate [39] and the two cysteines modified in the TruE substrate, LynD was anticipated to be extremely permissive of different amino acid residues surrounding the modified cysteine residue. In the TruE* peptide, both the entire core (five amino acids preceding the modification) and the follower (four amino acids after the modification) were included in the library, with the follower treated as core peptide rather than a structural element (similarly to PaaP follower in its library). Given the number of residues in the library, and the potentially high tolerance of diverse amino acids, a saturation mutagenesis library of all positions simultaneously was used, allowing the core sequence to be xxxxxCxxxx (SEQ ID NO: 112), where x is any amino acid. A single degenerate oligonucleotide, with all core and follower codons except the cysteine replaced by NNK, was used to build the library. In the resultant variants, peptides with more than one cysteine were screened out, since it was impossible to tell which ones were modified via LC-MS. Twenty-four variants were isolated and assayed, in addition to 10 variants that were synthesized to have charged and/or bulky polar residues flanking the modified cysteine (native flanking residues are usually small and/or hydrophobic). All of the custom/designed variants were well modified, showing that LynD tolerated charged or bulky polar side chains at the modification site. Of the 24 random variants, 17 were modified above the half-of-wild-type threshold. At all of the positions included in the library, tolerated amino acids were physiochemically diverse, consistently including 5-6 of the 6 physicochemical groupings used to classify amino acids (positive, negative, polar, aliphatic, aromatic, G/P). Based on this, the motif was trimmed to include only the positions adjacent to the modified cysteine. Those two positions were updated to allow 19 amino acids, all except cysteine, since modification of adjacent cysteines was not investigated (FIG. 11B).

### Tailoring Enzyme Tolerance to Core Mutations

The same expression/analysis pipeline described for leader-dependent modifying enzymes was applied to leader-independent tailoring enzymes. Tailoring enzymes do not bind recognition sites in the leader, instead they bind directly to the site of modification in the core, with specificity presumably determined by the amino acids around the modification. As such, these enzymes have no RS or RS spacing constraints, but do have core sequence constraints that can be elucidated similarly to the core constraints of leader-dependent modifying enzymes. To maintain consistency between all enzymes, expression conditions were equivalent to those described for modifying enzymes: peptides were expressed as a SUMO fusion and expressed and modified in TB media in 96-well plate format.

Of the nine enzymes selected for characterization (Table 5), five were leader-independent tailoring enzymes. One of the enzymes modifies the side chain of an internal peptide residue while others modify the C-terminal residue side chain or carboxyl group. In contrast to the leader-dependent modifying enzymes, where all were from different RiPP classes, three of the five tailoring enzymes came from lasso peptide clusters, highlighting the compatibility of lasso peptide tailoring enzymes have with heterologous expression in this platform. The tailoring enzymes catalyze diverse transformations and have been sourced from diverse bacterial species (Table 5):

(1) EpiD is an oxidative decarboxylase from the epidermin biosynthetic pathway, a type 1 lanthipeptide antibiotic identified from Staphylococcus epidermidis[105, 106]. It is an integral tailoring enzyme for formation of the aviCys macrocyclization, though without the other enzymes in the pathway the aviCys cycle is not formed and decarboxylation results in an enethiolate [107], with a corresponding loss of mass of −46 Da. This modification is valuable both for its potential for forming constrained aviCys macrocycles[6] when combined with other enzymes and also for removing the carboxy group, decreasing polarity and potentially increasing membrane permeability[108, 109].

(2) PalS is a glycosyltransferase that catalyzes the class-defining glycosylation of pallidocin, a glycocin antibiotic[110]. In pallidocin, a cysteine is glycosylated, causing a gain of mass of +162 Da. Glycosylation can play diverse roles in small molecules, often used in antibiotics to inhibit peptidoglycan biosynthesis by glycopeptides[111] and now proposed as a strategy for improving peptide bioavailability during drug design[112].

(3) LasF is a methyltransferase from the lasso peptide antibiotic lassomycin[l13]. It methylates the carboxyl group on the C-terminus to form a methyl ester, causing a gain in mass of +14 Da. Similar to EpiD decarboxylation, the methyl ester is uncharged (unlike the carboxyl group), potentially aiding membrane permeability[108, 109].

(4) ThcoK and (5) PadeK are both kinases from lasso peptide clusters that install 1-3 phosphates on the C-terminal serine of their respective peptides[80, 81]. Because multiple phosphate groups can be added, the gain in mass can be +80, +160, and +240, corresponding to +1, +2, and +3 phosphates, respectively. Naturally, their biological role is unknown, but synthetically they can be used to modify substrate pKa/log P properties or create phosphopeptide mimetics that act as signal transduction inhibitors[114]. Both ThcoK and PadeK were included to enable phosphorylation of a greater number of peptides by investigating two kinases with presumably different sequence constraints. Since these enzymes install a variable number of phosphates, any number of phosphates to be “modified” was considered, meaning that fraction modified is the fraction of peptide that has 1, 2, or 3 phosphates installed.

Each of these tailoring enzymes catalyze a mass shift that can be assayed via LC-MS, in the same manner that leader-dependent modification was assayed. The five tailoring enzymes and their respective wild-type precursor peptides were first assayed for modification (FIG. 46). Fraction of peptide modified varied between the enzymes (0.29-1.0). PadeK was the only poorly modified enzyme, which was surprising since both previous work[81] and its homolog ThcoK showed efficient modification (FIG. 46). Peptide-enzyme pairs had similar modification between replicates (fraction modified standard deviation of 0.026-0.081), except PalA modification by PalS, which had a standard deviation of 0.58. The large variance between PalA replicates was caused by the detection limit of the LC-MS: poor LC-MS signal was observed for wild-type PalA peptide due to proteolytic cleavage of the leader by endogenous E. coli proteases. This caused full length (uncut) peptide to be low abundance (near the detection limit of the LC-MS), and in two replicates unmodified peptide did not pass detection thresholds (fraction modified is 1.0) while in the third replicate both modified and unmodified peptide did not pass (fraction modified is assigned 0.0). This problem was solved by removal of the leader (and most of the core peptide) during elucidation of the core motif, described further below.

Similar to leader-dependent modifying enzymes, core motifs were elucidated using scanning site saturation mutagenesis. Since tailoring enzymes do not require the leader (or a majority of the core), most of the precursor peptide was truncated to investigate only those residues surrounding the modification. Each peptide library was limited to eight varying positions. For tailoring enzymes that modified the amino acid side chain (PadeK, ThcoK, and PalS), the modified residue was not included in the library since it was necessary for modification, so the total peptide size was truncated to 9 amino acids. For the two enzymes that modified the carboxy group on the C-terminus (LasF and EpiD), the C-terminal residue was included in the library, so the total peptide size was truncated to the C-terminal 8 amino acids. The positions were numbered based on their position in the wild-type (full-length) core, not their position in the truncated version.

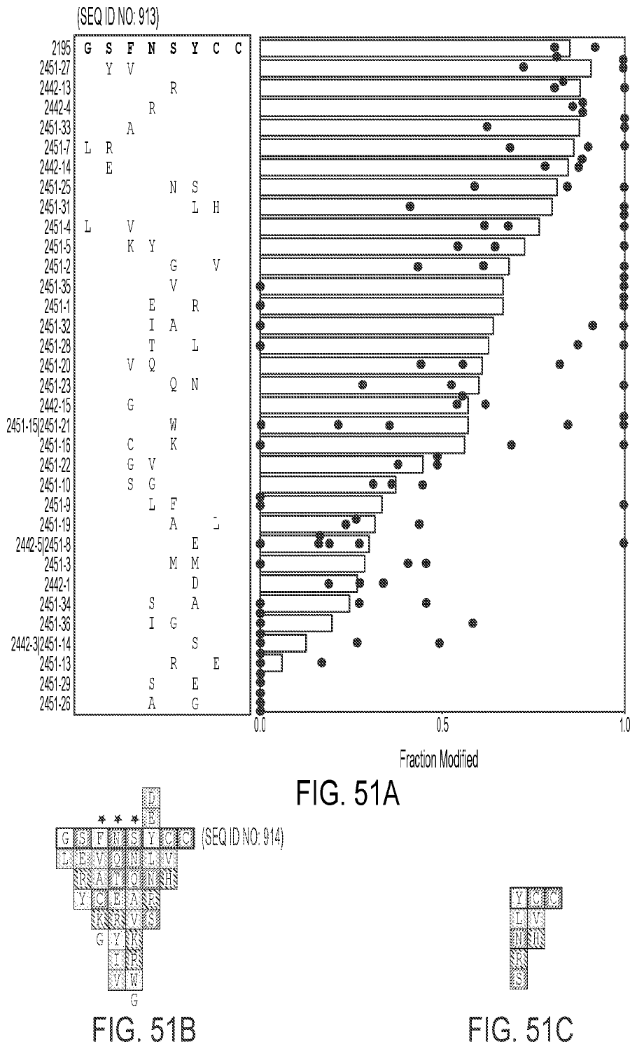

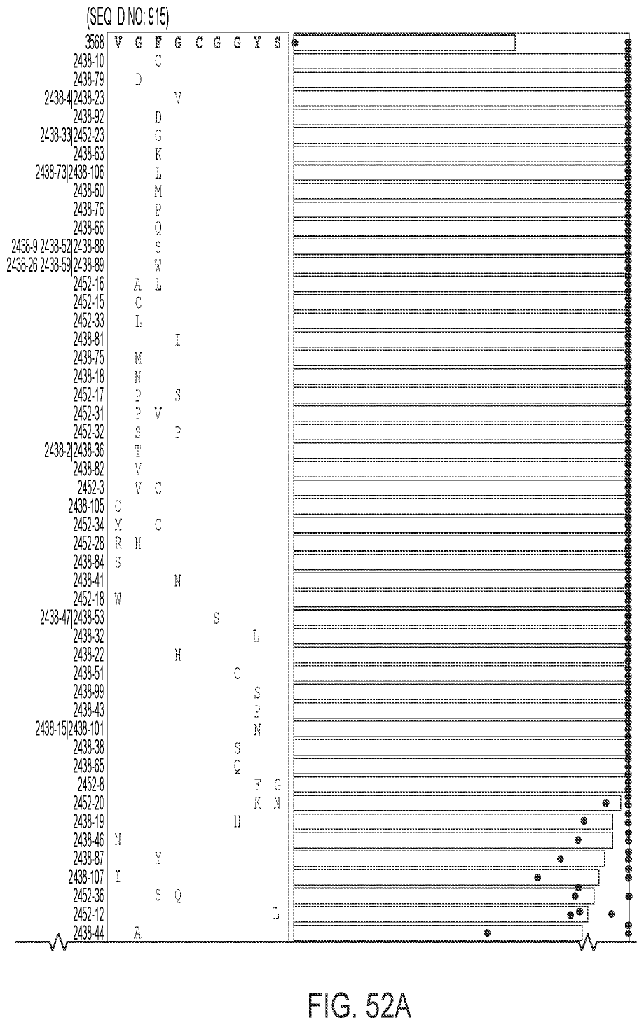

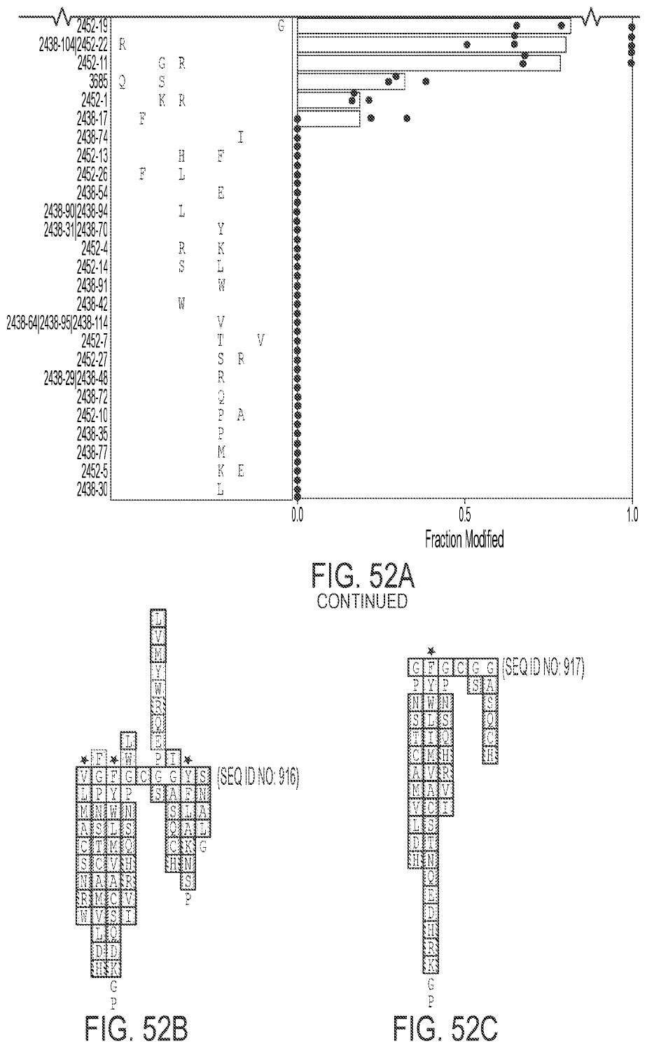

Initial libraries varying single amino acids at a time (like those used with TgnB, PlpXY, and PaaA) resulted in variants that were well modified (FIGS. 51A-51C, 52A-52C, 53A-53C, 54A-54C, and 55A-55C), indicating that the core motifs were very permissive. To accelerate exploration of amino acid sequence space, libraries that had multiple mutated positions were also designed. Several library architectures were used, including NNK-NNK (two adjacent randomized positions, abbreviated dSSSM for double SSSM), NNK—NNK—NNK (three adjacent randomized positions, abbreviated tSSSM for triple SSSM) or NNK-www-NNK (two randomized positions flanking a wild-type residue, w denoting a wild-type nucleotide, abbreviated dfSSSM for double flanking SSSM). These architectures were scanned through the truncated core and pooled such that variants had 2-3 mutated AAs at a random location. To build the motifs (FIG. 11C), the same cut-off of 50% of modification of the truncated wild-type peptide sequence was used for acceptance of amino acids at each position. When building summaries of tolerated amino acids at each position, unallowed amino acids were assigned using only single-mutation data, since the amino acid responsible for decreasing modification below the 50% threshold could not be determined when there were multiple mutations in a variant.

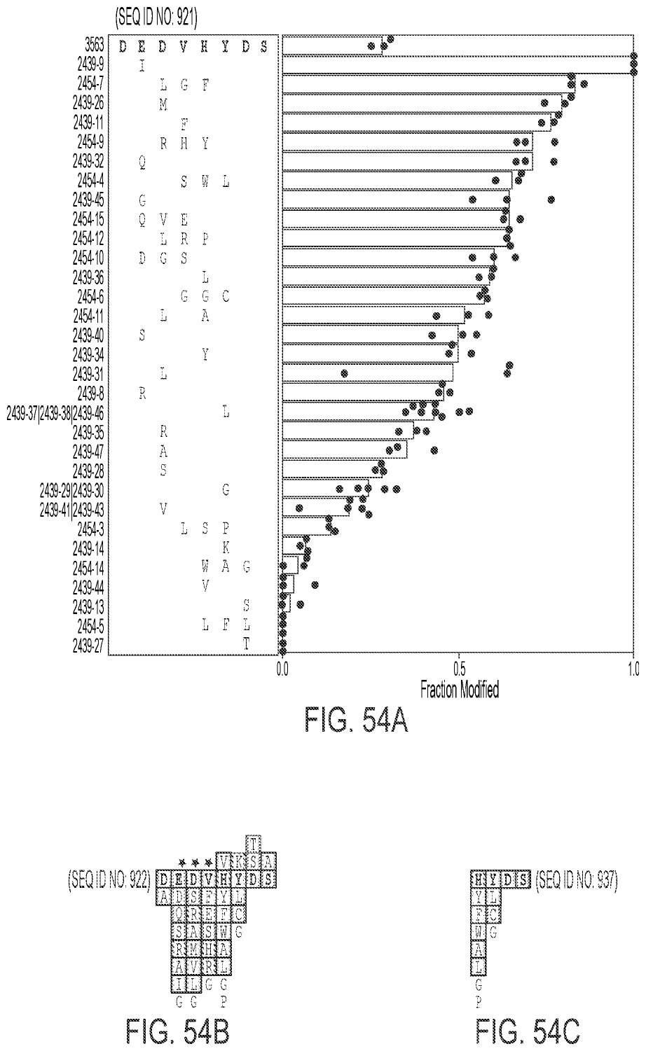

Finally, each motif was analyzed and minimized based on tolerated amino acids at each position. If every observed mutant at a position was accepted in the tolerance summary, and those tolerated amino acids spanned 4+ of the 6 physicochemical amino acid classes used, the position was annotated as unconstrained and allowed to be any amino acid. Unconstrained positions on the edge of a motif could then be removed from the motif entirely. During golden-gate/typeIIs assembly of the libraries, assembly bias that lowered the number of amino acid variants at the N- and C-termini of the library was observed, so terminal positions were often removed from the motif if they didn't meet the 4+ criteria above, but had unconstrained positions between them and the modified residue. For example, in the PadeK tolerance summary (FIG. 54B), core positions E17, D18, and V19 met the criteria to be unconstrained, but position D16 at the N-terminus only had one mutant observed due to assembly bias. It is unlikely that position 16 was constrained, while 17, 18, and 19 were not, so positions 16 -19 were removed from the PadeK motif (FIG. 11C).

The EpiA peptide was truncated to include the eight C-terminal residues (positions 15 through 22). EpiD modification was investigated using sSSSM, dSSSM, and dfSSSM libraries, each of which were cloned separately and a total of 33 variants isolated and assayed between the libraries. For many of the variants, the replicates varied more than what was observed for other enzyme peptide variants. Analysis of the raw chromatograms showed large peaks that were above the detection limit, but the spectra were noisier than spectra from other peptides/enzymes, for unknown reasons. Despite the lower quality data, trends were visible: mutations close to the N-terminus were observed to be well modified and those close to the C-terminus (modification site) were poorly modified. Position 20 did not tolerate negatively charged aspartate/glutamate amino acids, while hydrophobic (L), polar (S, N, and Y), and positively charged (R) amino acids were tolerated. Positions 17, 18, and 19 were found to be very permissive and all mutations at positions 15 and 16 were tolerated, so positions 15-19 were removed from the core motif, which is shown in FIG. 11C. EpiD substrate tolerance has been investigated in vitro, using neutral loss mass spectrometry to measure modification[54]. The final three residues were also annotated as important for modification, but observed V, I, L, F, Y, and W tolerated at position 20 and A, S, V, T, C, I, and L tolerated at position 21 (with all other amino acids measured and not tolerated). None of the tolerated amino acids were not tolerated in the variants tested, but several additional amino acids were observed to be tolerated at both of those positions (N, R, and S at position 20 and H at position 21). The discrepancy may be explained by non-additive effects described earlier—those amino acids were observed as tolerated based on variants that included multiple mutations, but they may not be tolerated in isolation. Indeed, Y20S was not tolerated in isolation, but modification was recovered with S19N mutation (FIG. 51A).

The PalA peptide was truncated to include the four amino acids to either side of the glycosylated cysteine (9 amino acids total). Three libraries were designed: sSSSM, dSSSM, and dfSSSM, with 74 total variants assayed for modification by PalS (Supplementary Note 10). A majority of variants (40) were 100% modified, with only 14 variants showing intermediate levels of modification and the remaining 20 not tolerated. Of those that weren't tolerated, all but two included mutations flanking the modified cysteine (positions 24 and 26). The remaining two were G22F and G27I single mutation variants, both surprising given the diverse amino acids tolerated at both of those positions. While there were multiple examples of variants with overlapping amino acids at a position, investigating non-additivity was impossible, since most variants were not at quasi-steady state but were fully modified. Mutation S29G had a lower fraction modified (0.81) than S29G with Y28F (1.0), but was within the S29G standard deviation of +/−0.24. In another example, F23K was fully modified while F23K with G24R as poorly modified (0.19). Assuming additivity, G24R was the offending mutation, except F23G with G24R was well modified (0.79). This may be an example of non-additivity, but because the F23K single mutant variant was fully modified it's possible that the F23K mutation was detrimental to modification, but not enough to lower the fraction modified below 1.0. Only when combined with another slightly detrimental mutation, G24R, did F23K mutation bring modification down significantly. Without a clear indication of non-additivity, the core tolerance summary was assembled using all the variants, observed positions 21, 23, and 28 to be unconstrained, and updated the core motif to include positions 22-27 (FIG. 11C).

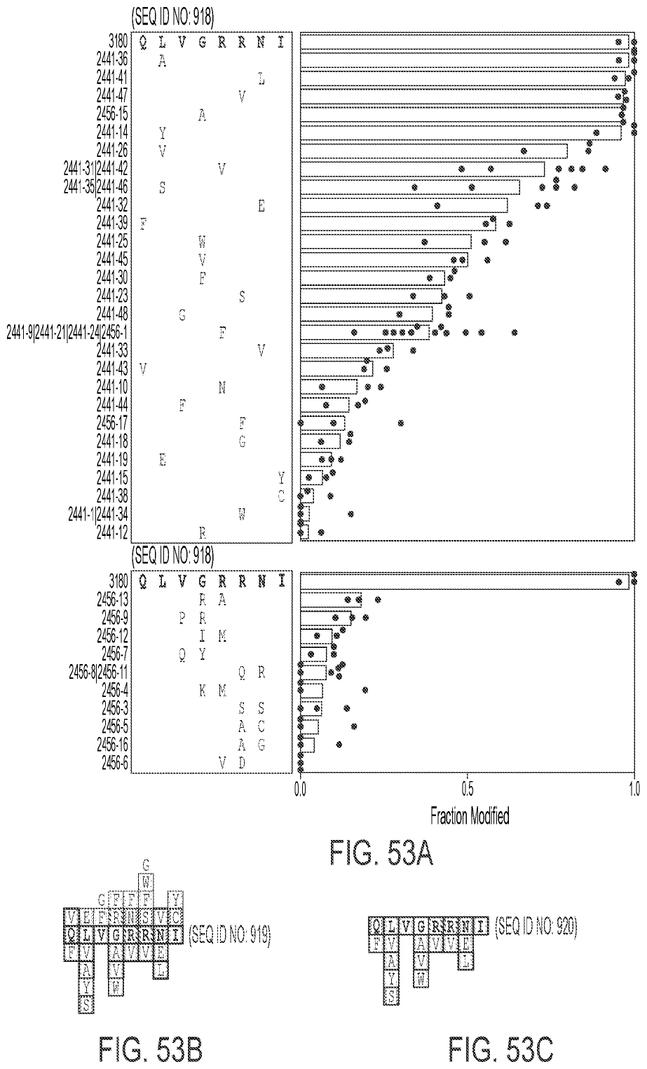

The LasA peptide was truncated to include the C-terminal eight amino acids, all of which were varied in the library. Both sSSSM and dSSSM libraries were constructed, with 37 variants isolated and assayed for modification. Mutations to LasF had greater impact on the activity of the enzyme compared to variants for other tailoring enzymes. None of the variants with multiple mutations were well modified, and only 5 single-mutant variants had wild-type levels of modification. Hydrophobic amino acids (A, V, L, F, and W) were generally allowed in all positions. Mutation of the C-terminal isoleucine to tyrosine and cysteine was not tolerated, in agreement with data for a LasF homolog showing mutation of the C-terminal residue led to a 4-fold reduction in methylation. The variant data was used to build the core tolerance summary (FIG. 53B) and core motif (FIG. 11C), with no other physicochemical or positional trends.

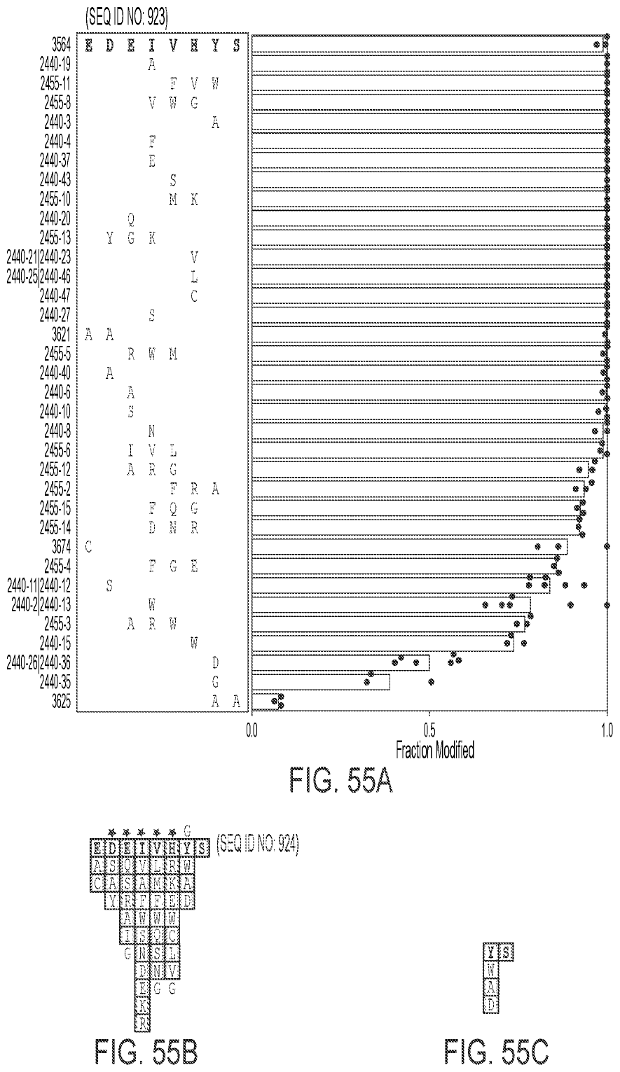

PadeK and ThcoK were both truncated to include the C-terminal nine amino acids, with the final serine not included in the library since its side chain is modified. Both of these enzymes were very tolerant to diverse core sequences, so sSSSM, dSSSM, dfSSSM, and tSSSM libraries were all used to elucidate core constraints. In total, 31 PadeA variants and 34 ThcoA variants were tested. ThcoK was the most tolerant enzyme investigated: only one variant was below the modification threshold, with the mutation adjacent to the modified cysteine. Positions 16 through 21 all passed the criteria for being unconstrained, so positions 15 through 21 were removed from the motif, leaving only the modified serine, and the preceding residue. PadeK was more constrained: it only showed high specificity at the penultimate core residue and at core positions 22 and 21, respectively (adjacent to the ultimate/modified serine) (FIG. 11C). The ThcoK motif was decreased to the final two residues and the PadeK motif was decreased to the final four residues. Only 2 of 20 random single-mutation variants in ThcoA decreased ThcoK modification below 50%, and both were adjacent to the modified residue (FIG. 55A).

Design of Peptides with Multiple PTMs

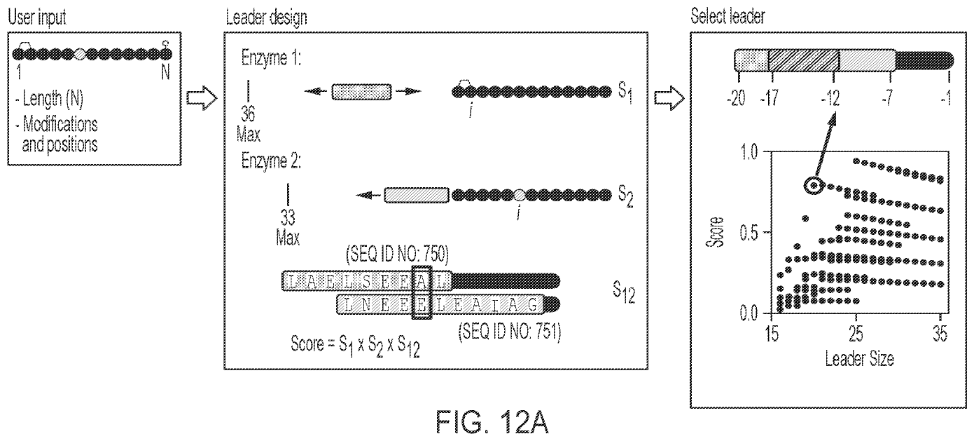

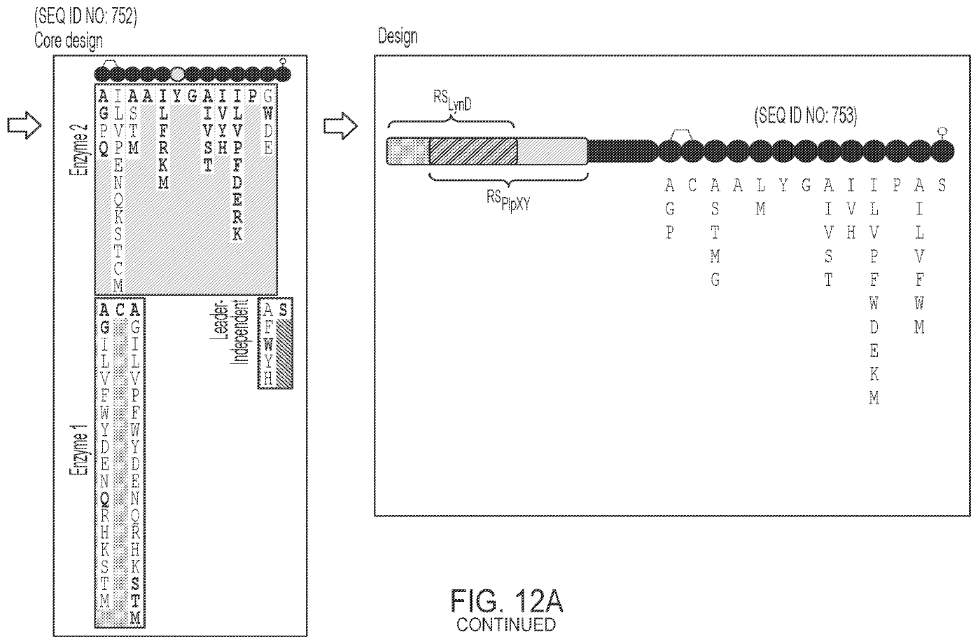

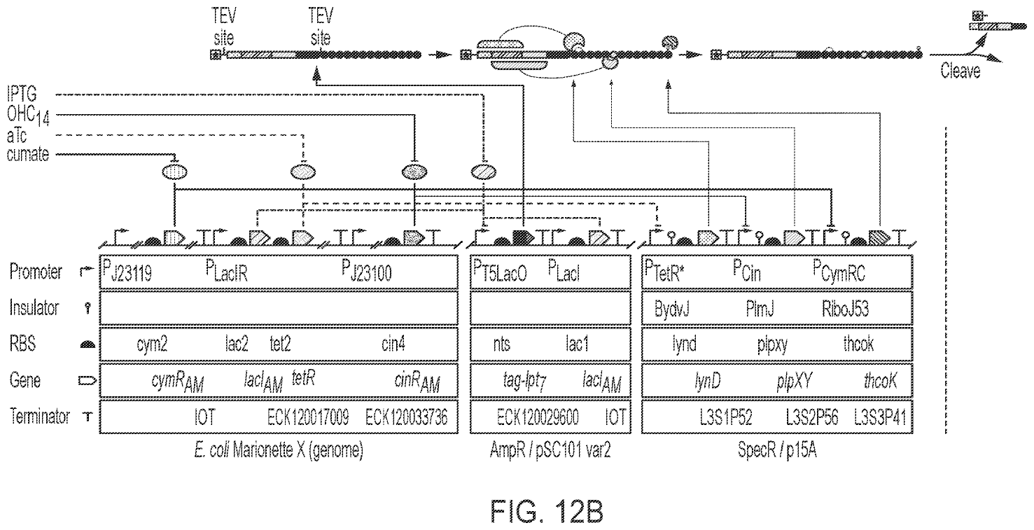

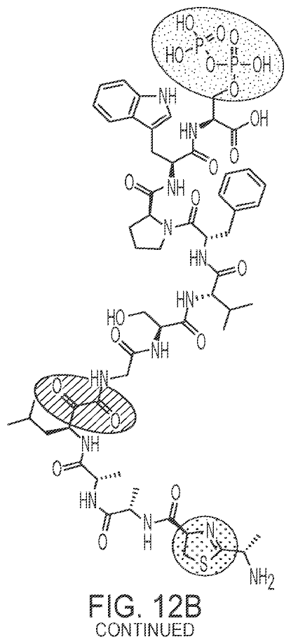

A design algorithm was developed to create a library of core variants enriched for a desired modification pattern (FIG. 12A). Each modification imposes new constraints on the precursor peptide sequence. To this end, the algorithm had two objectives. First, the leader must place the RS sequences with the correct spacing to the amino acids they modify. If present, gaps between RSs and/or between the RS and the core must be filled by the algorithm. Second, the constraints on the core sequence have to be combined to create a pattern of tolerated amino acids for all of the modifications. The core also needs to be scanned to predict potential off-target modifications.

Leader design proceeds by moving the RS sequences with respect to the core and calculating their contribution to a scoring function. The maximum leader length is a parameter that can be set in the algorithm, with a default value of L=40 amino acids. The score S of RS placement m is the predicted effect of RS-to-modification distance d compared to optimal distance d₀.

$${S}_{m} {e}^{\frac{- {κ}_{1} H ( d - {d}_{0} ) ( d - {d}_{0} ) - {κ}_{2} ( 1 - H ( d - {d}_{0} ) ) ( d - {d}_{0} )}{2 RT}} \qquad ( Equation 4 )$$

which is bounded to the range 0-1 (inclusive). The total score for a RS placement in a leader p for a set of M enzymes is defined as

Sₚ=Πₘ₌₁^{N}Sₘ (Equation 5)

The algorithm then seeks to identify the optimum p that maximizes the score. This can be found simply by enumerating all possible placement combinations of the RS sequences.

There are several use cases in which it is beneficial to save space by overlapping the RS sequences, as sometimes occurs in natural leaders. For instance, the constraints on d might be too rigid to separate them. It could also free other space in the leader for additional enzymes to bind. Finally, shorter leaders could facilitate the use of specific DNA oligosynthesis techniques in building a library. To this end, an algorithmic approach was developed to evaluate overlapping RS sequences. If two RS sequences could overlap without any amino acid mismatches, then this was done without penalty. However, in most cases, overlap would require an imperfect RS for at least one enzyme. To capture this, an additional term was calculated to modify the score,

$${S}_{mn} = \frac{( a - z ) ( b - z )}{ab} . \qquad ( Equation 6 )$$

In Equation 6, a and b are the lengths of RS1 and RS2 and z is the number of mismatched residues (BLOSUM62 score less than or equal to 0) in the overlap of the two recognition sites. The fraction was bounded to the range of 0-1 (inclusive) and simply included in the product of terms for the total score (Equation 5). If more than two RSs were being combined, more than one pair of RSs may overlap, and Sₘₙ was calculated for each overlapping pair and included with Equation 5. At mismatched overlapping RS positions, a random choice between the two possible amino acids can be made, or one RS can be given priority over the other in selecting the amino acid.

Typically, if tolerated, a TEV protease site was included between the leader and the core so the core could be released and recovered after purification. When used, the TEV sequence constraints were treated as an additional leader-dependent modifying enzyme. The six amino acid TEV sequence ENLYFQ (SEQ ID NO: 111) was added as an RS, with fixed placement (high κ constants), such that it contributed to the calculation of Sₚ. TEV cleavage occurs after this sequence and was permissive to different amino acids at the first position of the core, except P, and reduced efficiency for L/E/I/V [115]. This core constraint was added as a core motif, with placement specified at position 1 of the core. In addition to this, there may be a gap between the RS sequences or between the last RS and the core. There are multiple options for filling these gaps provided by the algorithm: (1) GGS repeats; (2) choosing random amino acids (additional sequence constraints can be optionally added at any leader position); (3) spacer sequences taken from wild-type leaders of the enzymes being combined; and (4) nothing, the leader is returned with gaps to be filled in manually.

The final step was to design the core (FIG. 12A). First, the positions to be modified were fixed. The rules for each enzyme were then aligned to these positions. They were then combined to create a motif over the length of the sequence, where an amino acid was only allowed at a residue if it was allowed by all the enzymes at that position. Additional constraints can be added by the user, at any position, to encode a pharmacophore of interest, limit combinatorial complexity, or influence hydrophobicity or other physicochemical properties. Positions not restricted by an enzyme sequence constraint can be any amino acid. A library can then be generated of size N that randomly assigns amino acids from those allowed at each position. Optionally, this library can be filtered to remove sequences that have motifs at off-target sites that could potentially be modified by the enzymes. The output can serve to guide pooled oligo synthesis strategies.

### Forward Design of a Synthetic RiPP

The algorithm was applied to design precursor peptides that can be modified by four enzymes: two leader-dependent modifying enzymes (LynD and PlpXY), one tailoring enzyme (ThcoK), and TEV protease (FIGS. 12A-12B). The core was defined to be 13 amino acids, with a thiazoline, excised tyrosine and phosphorylated serine at positions 2, 6 and 13, respectively. TEV cleavage was specified at position 1. The LynD, PlpXY, and TEV recognition sequences had to be combined into the leader sequence. It was hypothesized that the LynD and PlpXY RSs could overlap because the alanine-scan variants through the RSs for both enzymes were well tolerated (FIGS. 50A and 48A), indicating sequence plasticity. Then, the scores of all combinations of the LynD and PlpXY sequences, including overlaps, were calculated. This is shown in FIG. 12A, where there is a pareto-optimal boundary between the best scores and minimum leader size. A variant was chosen that had a high score but shortened the leader by having the RS sequences overlap by six amino acids (two mismatches).

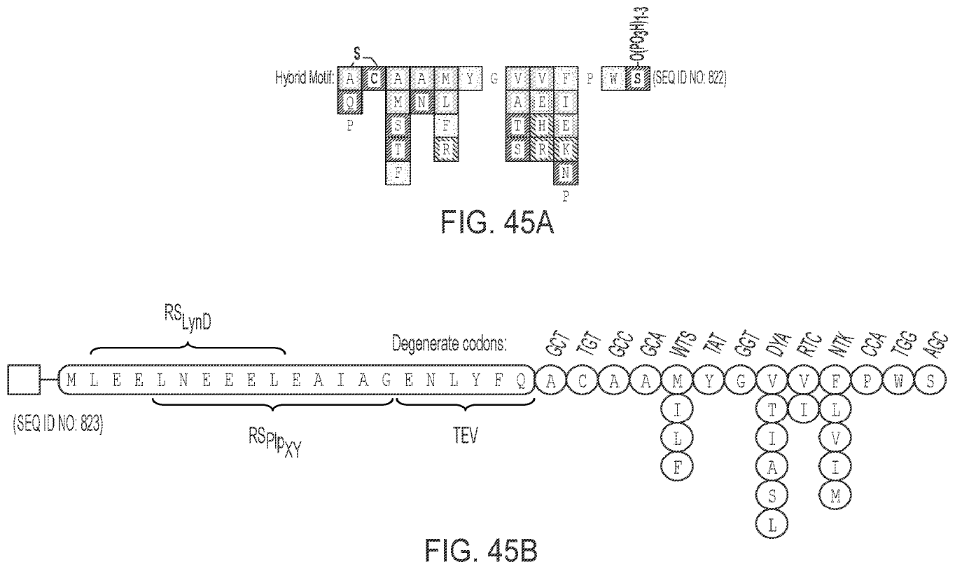

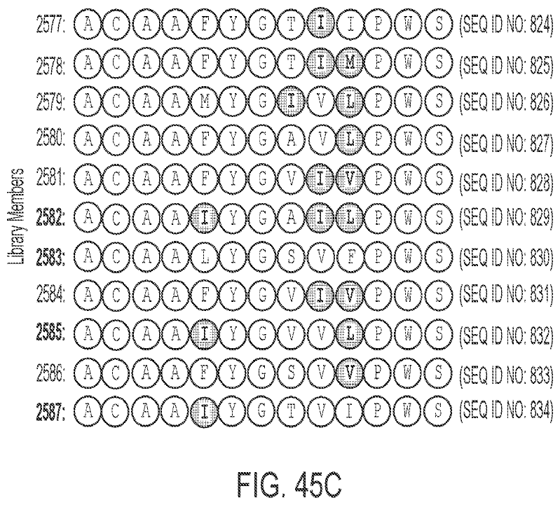

A core motif was then designed by combining the rules associated with the three enzymes and including the restriction from the TEV protease that a proline cannot appear in the first position. Considering the variability allowed at each position, this resulted in 21,000 peptides that conformed to the rules. This was in contrast to the ˜10¹³ peptides that would result from all 20 amino acids being allowed at all non-modified positions. An oligo pool was built and designed to access a subset of the allowed peptides and cloned and sequence verified one that matched the enzyme restrictions and ten that had imperfect matches (FIGS. 45A-45C). To enable modification with multiple enzymes, a Marionette derivative of NEB Express E. coli was used, such that all inducible systems were encoded in the genome. Enzyme genes lynD, plpXY, and thcoK were placed under aTc-, OHC14-, and cumate-inducible systems, respectively, and assembled together onto a spectinomycin-resistant p15A backbone (FIG. 12B). The engineered leader-core was cloned under the same IPTG-inducible expression plasmid used above, which included its own copy of lac (in addition to the lacI encoded in the Marionette genome cassette), but this did not affect peptide expression. This created an artificial biosynthetic gene cluster of four genes, all under independent, inducible, control.

Expression and peptide modification was investigated in the same manner as for individual enzymes. Each of the eleven peptide plasmids were co-transformed with the multi-enzyme plasmid. Overnight cultures were diluted 1:100 into TB media, fully induced after 3 hours at 30° C., and incubated for 20 hours. Cultures were then lysed, affinity purified, and assayed via LC-MS.

All possible combinations of modification were searched (dehydration from LynD modification (−18 Da), tyramine excision from PlpXY modification (−135 Da), and phosphorylation from ThcoK modification (+80 Da)). For four of the peptides, masses were identified that matched expected triple-modification masses, suggesting a success rate of 80% for the hybrid core motif. The peptide variant with the highest fraction of triply modified peptide was selected for validation.

The co-transformed strain was struck out, and three colonies were individually grown up at small scale, affinity purified, and TEV cleaved. The final molecule was assayed via LC-MS/MS, where the mass and observed fragments matched the expected peptide structure.

### DISCUSSION

This Example abstracted the substrate preferences of RiPP enzymes as “rules,” applicable to the constraint-based design of precursor peptides. Computational design can be used to guide the selection of enzymes to decorate a natural product [116], identify scaffolds to splice in a binding sequence [61, 117], or design large screening libraries enriched in modified peptides [62]. While RiPPs are generally very tolerant, the success rate declines rapidly as more constraints are added. For the example in FIGS. 12A and 12B, the enzyme rules estimated that only 1 in 300 million random peptides (holding the modified amino acids constant) would lead to a triply modified peptide. A library built according to these rules would contain 31,500 predicted unique compounds. Creating a large library for an exact set of core sequences has been historically difficult, where construction required random mutagenesis (e.g., NNK), but it is now trivial using custom pooled DNA synthesis services [118].

Chemical retrosynthetic planning algorithms use “rules,” extracted from the literature, to represent how a chemical moiety will be converted by a reaction [13, 119-121]. There is a trade-off between accuracy and path discovery: if every rule is specific to only one chemical, this would be the most reliable, but it would not be possible to predict paths to new chemicals. Algorithms balance these needs by specifying rules with respect to the number of atoms from the reaction center n; if n=0, then it is just the reaction itself and as n gets larger, this increases the accuracy as more of the chemical context is incorporated into the rule. This approach has been extended to enzymes using the same rules-based method of defining allowable enzyme substrates based on the substrate reaction center and surrounding atoms/functional groups [13].

Considering rules for RiPP enzymes, simply defining the chemistry performed by an enzyme and assuming perfect promiscuity for the other core positions is the philosophical equivalent to n=0. This assumption has implicitly appeared in the literature for RiPP design when highly tolerant enzymes were combined without restricting the core sequence [11, 23-25, 27]. Simultaneously, other retrosynthesis studies have engineered multiply modified peptides by generating peptide chimeras, with an enzyme effectively modifying its wild-type substrate [74, 76, 77], the equivalent of a large and un-engineerable n-value. The rules defined in this Example are the next level of constraints, representing the minimal information to capture substrate specificity. However, they incorporate a number of assumptions, including the additive combination of amino acid tolerances derived from single-mutant data. Indeed, incidences of non-additive compensatory effects from multiple mutations were observed. The next level of accuracy in rules could account for higher-order effects requiring more sequence knowledge of the core, such as charge, hydrophobicity, secondary structure, and loop entropy, all of which have been cited as important in determining RiPP enzyme specificity [22, 26, 42, 45, 47, 76, 122]. Similarly, in the leader it was assumed that recognition sites and spacing alone were determining factors of modification, but TgnB recognition site spacing variants varied in modification based solely on spacer sequence, indicating that leader sequence outside of the recognition site may affect modification (FIG. 10D), possibly due to spacer structure/flexibility. Mapping additional leader/core rules could be aided with artificial intelligence, which has been applied for this purpose to define rules for chemical retrosynthesis and has been applied to predict protein-protein interactions[121, 123].

However, many RiPP enzyme have properties, or gaps in knowledge, that make their function difficult to capture as a “rule.” Enzymes with wide RS spacing tolerance are often progressive, with difficult-to-predict behavior where single leader mutations change the modification pattern [20, 32, 34]. Kinetics are also a complicating factor, as enzymes in the same pathway can have orders-of-magnitude differences in time scales, from less than an hour to days[10, 33, 36, 124]. Imperfect leader sequences have been observed to alter enzyme kinetics, not just binding[33]. The order of operations also matters for cases in which later modifications require earlier ones to occur, for example, when a cyclization or epimerization orients an amino acid such that it is accessible for a subsequent modification[20, 30, 32, 61, 125, 126]. Tailoring enzymes can require that the released core peptide adopt a particular shape [42, 47, 52, 127].

This Example provides a new type of RiPP enzyme mining effort that differs from the approach of discovering new bioactive compounds by finding and reconstructing entire gene clusters from metagenomics data [65, 128]. Screens can be established to identify modifying enzymes along with simple approaches to define the minimal rule sets for their use. Because the goal is to combine them into a pathway, these enzymes need to be screened under a common set of conditions, whether it be in vivo or in vitro [76] and jettisoning those that do not work in this standardized context or that exhibit odd or unpredictable behaviors. These conditions may not reveal the precise role of enzymes in nature, but they provide the necessary information for forward design of artificial pathways. The “ideal” enzyme for retrosynthesis can also begin to be defined. One might think that it is a very tolerant enzyme regarding spacing to the modification, but broad substrate specificity can lead to unpredictable modification of multiple core residues and slow kinetics [33]. Instead, when given the option, it may better to have multiple enzymes on hand that differ in the distance from the RS where they modify their residue, such as appears to be the case in bottromycin biosynthesis [21]. Enzyme engineering, such as directed evolution, could be used to widen or tune substrate specificity specifically for the purpose of retrosynthesis. On last count, there are 300,000 RiPP clusters in the genomic databases with 4.6 million enzymes spanning ˜40 classes [129-134]. Finding subsets that work well together and characterizing their rules under common conditions would enable an enormous functional space to be algorithmically or combinatorially explored, providing unprecedented access to an emerging therapeutic modality: medium-sized constrained molecules, which are already showing promise for disrupting protein-protein interactions and other therapeutic targets that have traditionally been considered “undruggable”.

### Materials and Methods

### Strains, Plasmids, Media, and Chemicals.

E. coli NEB 10-beta (C3019I, New England BioLabs, Ipswich, Mass., USA) was used for all routine cloning. E. coli NEB Express (C2523I, New England BioLabs, Ipswich, Mass., USA) was used to express precursor peptides with single modifying enzymes, and the Marionette derivative of E. coli NEB Express (Marionette X) was used to express precursor peptides with multiple modifying enzymes. Plasmids for precursor peptide expression and modifying enzyme expression were used as follows: precursor peptide genes used a pSC101 origin variant (var 2) [87] and single modifying enzyme plasmids contained p15A origins of replication and kanamycin resistance. Plasmids with multiple modifying enzymes contained p15A origins of replication and spectinomycin resistance. LB-Miller (B244620, BD, Franklin Lakes, N.J., USA) and TB (T0311, Teknova, Hollister, Calif., USA) supplemented with 0.4% glycerol (BDH1172-4LP, VWR, OH, USA) were used for peptide expression and modification. 2xYT liquid media (B244020, BD, Franklin Lakes, N.J., USA) and 2xYT+2% agar (B214010, BD, Franklin Lakes, N.J., USA) plates were used for routine cloning and strain maintenance. SOB liquid media (S0210, Teknova, Hollister, Calif., USA) was used for making competent cells. SOC liquid media (B9020S, New England BioLabs, Iwsich, Mass., USA) was used for outgrowth. Cells were induced with the following chemicals: cuminic acid ≥98% purity from Millipore Sigma (268402, Millipore Sigma, Saint Louis, Mo., USA) added as 1000× stock (200 mM) in EtOH or DMSO; isopropyl β-D-1-thiogalactopyranoside (IPTG) ≥99% purity (I2481C, Gold Biotechnology, Saint Louis, Mo., USA) added as 1000× stock (1 M) in water or DMSO. Cells were selected with the following antibiotics: kanamycin (K-120-10, Gold Biotechnology, Saint Louis, Mo., USA) as 1000× stock (50 mg/ml in H2O); carbenicillin (C-103-5, Gold Biotechnology, Saint Louis, Mo., USA) added as 1000× stock (100 mg/ml in H2O); spectinomycin (22189-32-8, Gold Biotechnology, Saint Louis, Mo., USA). Liquid chromatography was performed with Optima Acetonitrile (A996-4, Thermo Fisher Scientific, MA, USA) and water (Milli-Q Advantage A10, Millipore Sigma, Saint Louis, Mo., USA) supplemented with LC-MS Grade Formic Acid (85178, Thermo Fisher Scientific). The following solvents/chemicals were also used: Ethanol (V1001, Decon Labs, King of Prussia, Pa., USA), Methanol (3016-16, Avantor, Center Valley, Pa., USA), dimethyl sulfoxide (DMSO) (32434, Alfa Aesar, Ward Hill, Mass., USA), Imidazole (IX0005, Millipore Sigma, Saint Louis, Mo., USA), sodium chloride (X190, VWR, OH, USA), sodium phosphate monobasic monohydrate (20233, USB Corporation, Cleveland, Ohio, USA), sodium phosphate dibasic anhydrous (204855000, Acros, N.J., USA), guanidine hydrochloride (50950, Millipore Sigma, Saint Louis, Mo., USA), tris (75825, Affymetrix, Cleveland, Ohio, USA), TCEP (51805-45-9, Gold Biotechnology, Saint Louis, Mo., USA), and EDTA (0.5M stock, 15694, USB Corporation, Cleveland, Ohio, USA). DNA oligos and oligo pools were ordered from Integrated DNA Technologies (San Francisco, Calif., USA) and enzymes and peptide plasmids were assembled/cloned in-house or synthesized by Twist Biosciences (San Francisco, Calif., USA). Enzymes and peptides were codon optimized using an in-house optimization tool.

### Peptide Expression and Purification.

Saturated cultures in LB were diluted 1:100 into 1 ml TB in deep well plates, incubated for 3 hours (Multitron Pro, 30° C., 900 r.p.m.), supplemented with appropriate inducers, and incubated for an additional 20 hours (Multitron Pro, 30° C., 900 r.p.m.). For purification, plates were centrifuged (Legend XFR, 4,500 g, 4° C., 20 min), pellets were resuspended in 850 μl lysis buffer (5 M guanidinium hydrochloride, 300 mM NaCl, 50 mM sodium phosphate, pH 7.5), frozen (liquid nitrogen, −196° C.), thawed (Multitron Pro at 37° C., 900 r.p.m), and clarified via centrifugation (Legend XFR, 4,500 g, 4° C., 40 min). Peptides were affinity purified using His MultiTrap TALON plates (29-0005-96, GE Life Sciences), following manufacturer instructions, using 1×500 μl water and 2×500 μl lysis buffer for column equilibration, 2×500 μl wash buffer (300 mM NaCl, 50 mM sodium phosphate, 5 mM imidazole, pH 7.5), and 1×200 μl elution buffer (300 mM NaCl, 50 mM sodium phosphate, 150 mM imidazole, pH 7.5).

### Liquid Chromatography/Mass Spectrometry.

All chromatography was performed using mobile phases ACN (acetonitrile supplemented with 0.1% formic acid and 0.1% water) and water (supplemented with 0.1% formic acid). LC-MS was performed on one of two mass spectrometers: “QQQ” is an Agilent 1260 Infinity liquid chromatograph with binary pump configured in low-dwell volume mode, high-performance autosampler chilled to 18° C., and column oven, coupled to an Agilent 6420 QQQ mass spectrometer equipped with an Agilent electrospray ionization (ESI) source; nitrogen gas is supplied by a Parker Nitroflowlab and ESI source parameters are 350° C. gas temp at 12 L/min flow rate, 15 psi nebulizer voltage, 4000 V capillary voltage, 135 V fragmentor voltage, and 7 V cell accelerator voltage. “QTOF” is an Agilent 1260 Infinity II liquid chromatograph with binary pump configured in low-dwell volume mode and column oven set to 40° C., coupled to an Agilent 6545 QTOF mass spectrometer equipped with an Agilent electrospray ionization (ESI) source; nitrogen gas is building supplied and ESI source parameters are 350° C. gas temperature, 12 L/min gas flow, 30 psig nebulizer pressure, 350° C. sheath gas temperature, 8 L/min sheath gas flow, 3000 V capillary voltage, 1000 V nozzle voltage, 135 V fragmentor voltage, 15 V skimmer voltage, 600 V Oct 1 RF Vpp; the mass spectrometer was run in MS mode with reference mass enabled and tuned in positive mode with standard mass range (3200 m/z) and 2 GHz extended dynamic range.

### LCMS Data Analysis and Peak Integration.

mzXML files were parsed and imported into python to a long-form pandas dataframe and filtered for signals between 1-6 min and 500-2,500 Da. For each extract, the expected molecular weight of unmodified, modified, and partially modified (if applicable) peptides were calculated. For each molecular weight, all charge state [M+xH]^{x+} (x is number of protons/charges) masses were calculated and extracted as an EIC with a mass window of +/−5/x Da for extracts analyzed with “QQQ” and 2/x Da for extracts analyzed with “QTOF”. Charge state EIC intensities were summed together at each timepoint to generate an extracted compound chromatogram (ECC). If present, an ECC peak is fit with a skewed gaussian with parameters peak area, retention time, peak width, and peak skew. Peaks are considered real/trustworthy based on the following criteria: greater than 8 charge states present/observed at the same retention time (+/−0.2 min) with at least 4 being consecutive charge states, only one “large” peak in the ECC (i.e. no peaks greater than 80% of the largest peak height in the chromatogram), and not more than 2 “small” peaks (i.e. <3 peaks greater than 40% of the largest peak height), peak skew between 0 and 1.5, peak width less than or equal to 0.25. Within an extract, “total peptide” is defined as the sum of the peak areas of unmodified, modified, and partially modified (if applicable) peptides if the modification mass shift is >15 Da and is defined as the sum of the peak areas of unmodified and modified peptides otherwise (due to overlapping isotope distributions). Fraction modified is defined as the modified peptide peak area divided by the “total peptide”. Peak integrations and masses for each extract are listed in Supplementary Table 6. All analysis is done in python 3.5 using pandas, scipy, numpy, and matplotlib libraries.

### REFERENCES FOR EXAMPLE 3

1. Van Arnam, Chemical Society Reviews, 2018. 47(5): p. 1638-1651
2. Vogt, and Künzler, Applied Microbiology and Biotechnology, 2019. 103(14): p. 5567-5581
3. Montalbán-López, M., et al., Natural Product Reports, 2021
4. Addison, W. N., et al., Biomaterials, 2010. 31(36): p. 9422-9430
5. Wallace, A. K., et al. Advanced Functional Materials, 2020. 30(30): p. 2000849
6. Morrison, C., Nat Rev Drug Discov, 2018. 17(8): p. 531-533
7. Müller, et al. Angewandte Chemie International Edition, 2007. 46(25): p. 4771-4774
8. Nicolaou, K. C., et al., Journal of the American Chemical Society, 2005. 127(31): p. 11176-11183.
9. Nicolaou, K. C., et al., Journal of the American Chemical Society, 2005. 127(31): p. 11159-11175.
10. Gu, and Schmidt, Accounts of Chemical Research, 2017. 50(10): p. 2569-2576.
11. Goto, Y. and H. Suga, Curr Opin Chem Biol, 2018. 46: p. 82-90.
12. Hudson, G. A. and D. A. Mitchell, Current Opinion in Microbiology, 2018. 45: p. 61-69.
13. Lin, G.-M., et al. Current Opinion in Systems Biology, 2019. 14: p. 82-107.
14. Chekan, J. R., et al. Proceedings of the National Academy of Sciences, 2019. 116(48): p. 24049-24055.
15. Burkhart, B. J., et al., Nature Chemical Biology, 2015. 11(8): p. 564-570.
16. Oman, T. J. and W. A. Van Der Donk, Nature Chemical Biology, 2010. 6(1): p. 9-18.
17. Morinaka, B. I., et al., Angewandte Chemie International Edition, 2014. 53(32): p. 8503-8507.
18. Morinaka, B. I., et al., Science, 2018. 359(6377): p. 779-782.
19. Arnison, P. G., et al., Nat. Prod. Rep., 2013. 30(1): p. 108-160.
20. Zhang, Z., et al., Journal of the American Chemical Society, 2016. 138(48): p. 15511-15514.
21. Crone, W. J. K., et al., Angewandte Chemie International Edition, 2016. 55(33): p. 9639-9643.
22. Bhushan, A., et al., Nature Chemistry, 2019. 11(10): p. 931-939.
23. Acker, M. G., et al. Journal of the American Chemical Society, 2009. 131(48): p. 17563-17565.
24. Bowers, A. A., et al., Journal of the American Chemical Society, 2010. 132(21): p. 7519-7527.
25. Bowers, A. A., et al., Journal of the American Chemical Society, 2012. 134(25): p. 10313-10316.
26. Tran, H. L., et al., Journal of the American Chemical Society, 2017. 139(7): p. 2541-2544.
27. Zhang, F. and W. L. Kelly, ACS Chemical Biology, 2015. 10(4): p. 998-1009.
28. Ozaki, T., et al., Nature Communications, 2017. 8(1): p. 14207.
29. Fleming, S. R., et al., Journal of the American Chemical Society, 2019. 141(2): p. 758-762.
30. Vagstad, A. L., et al., Angewandte Chemie International Edition, 2019. 58(8): p. 2246-2250.
31. Deane, C. D., et al., ACS Chemical Biology, 2013. 8(9): p. 1998-2008.
32. Rahman, I. R., et al., ACS Chemical Biology, 2020. 15(6): p. 1473-1486.
33. Thibodeaux, et al. Journal of the American Chemical Society, 2014. 136(50): p. 17513-17529.
34. Goto, Y., et al., Chemistry & Biology, 2014. 21(6): p. 766-774.
35. Li, C., et al. Mol. BioSyst., 2011. 7(1): p. 82-90.
36. Freeman, M. F., et al., Nature Chemistry, 2017. 9(4): p. 387-395.
37. Yu, Y., et al. Protein Science, 2013. 22(11): p. 1478-1489.
38. Cubillos-Ruiz, A., et al., Proceedings of the National Academy of Sciences, 2017. 114(27): p. E5424-E5433.
39. Sardar, D., et al., ACS Synthetic Biology, 2015. 4(2): p. 167-176.
40. Zhang, Q., et al., ACS Chemical Biology, 2014. 9(11): p. 2686-2694.
41. Van Der Velden, N. S., et al., Nature Chemical Biology, 2017. 13(8): p. 833-835.
42. Ruffner, D. E., et al. ACS Synthetic Biology, 2015. 4(4): p. 482-492.
43. Zong, C., et al. ACS Chemical Biology, 2016. 11(1): p. 61-68.
44. Mukherjee, S. and W. A. Van Der Donk, Journal of the American Chemical Society, 2014. 136(29): p. 10450-10459.
45. Tianero, M. D. B., et al., Journal of the American Chemical Society, 2012. 134(1): p. 418-425.
46. Hao, Y., et al., Proceedings of the National Academy of Sciences, 2016. 113(49): p. 14037-14042.
47. Vinogradov, A. A., et al., Nature Communications, 2020. 11(1).
48. Deane, C. D., et al., ACS Chemical Biology, 2016. 11(8): p. 2232-2243.
49. Rink, R., et al., Biochemistry, 2005. 44(24): p. 8873-8882.
50. Si, Y., et al., 2020, Cold Spring Harbor Laboratory.
51. Mordhorst, S., et al., Angewandte Chemie International Edition, 2020. 59(48): p. 21442-21447.
52. Ortega, M. A., et al., ACS Chemical Biology, 2014. 9(8): p. 1718-1725.
53. Ding, W., et al., Synth Syst Biotechnol, 2018. 3(3): p. 159-162.
54. Kupke, T., et al Journal of Biological Chemistry, 1995. 270(19): p. 11282-11289.
55. Zhang, Q. and W. A. Van Der Donk, FEBS Letters, 2012. 586(19): p. 3391-3397.
56. Young, S., et al., Chemistry & Biology, 2012. 19(12): p. 1600-1610.
57. Donia, S., et al., Chemistry & Biology, 2011. 18(4): p. 508-519.
58. Roh, H., et al., ChemBioChem, 2019. 20(8): p. 1051-1059.
59. Fleming, S. R., et al., Journal of the American Chemical Society, 2020. 142(11): p. 5024-5028.
60. Fuchs, S. W., et al., Angewandte Chemie International Edition, 2016. 55(40): p. 12330-12333.
61. Hegemann, J. D., et al., ACS Synthetic Biology, 2019. 8(5): p. 1204-1214.
62. Yang, X., et al Nature Chemical Biology, 2018. 14(4): p. 375-380.
63. Cotter, P. D., et al., Molecular Microbiology, 2006. 62(3): p. 735-747.
64. Schmitt, S., et al., Nature Chemical Biology, 2019. 15(5): p. 437-443.
65. Cebrian, R., et al., Design and Expression of Specific Hybrid Lantibiotics Active Against Pathogenic Clostridium spp. Frontiers in Microbiology, 2019. 10.
66. Young, T. S., Reviews in Cell Biology and Molecular Medicine.
67. Field, D., et al., Molecular Microbiology, 2008. 69(1): p. 218-230.
68. Rink, R., et al., Applied and Environmental Microbiology, 2007. 73(18): p. 5809-5816.
69. Appleyard, A. N., et al., Chemistry & Biology, 2009. 16(5): p. 490-498.
70. Boakes, S., et al., Applied Microbiology and Biotechnology, 2012. 95(6): p. 1509-1517.
71. Pan, S. J. and A. J. Link, Journal of the American Chemical Society, 2011. 133(13): p. 5016-5023.
72. Urban, J. H., et al., Nature Communications, 2017. 8(1).
73. Korneli, M., et al., ACS Synthetic Biology, 2021.
74. Burkhart, B. J., et al., ACS Central Science, 2017. 3(6): p. 629-638.
75. Huang, C.-F. and M. Mrksich, ACS Combinatorial Science, 2019. 21(11): p. 760-769.
76. Sardar, D., et al., Chemistry & Biology, 2015. 22(7): p. 907-916.
77. Van Heel, A. J., et al., ACS Synthetic Biology, 2013. 2(7): p. 397-404.
78. Ghodge, S. V., et al., Journal of the American Chemical Society, 2016. 138(17): p. 5487-5490.
79. Su, Y., et al., Applied Microbiology and Biotechnology, 2019. 103(6): p. 2649-2664.
80. Zhu, S., et al., FEBS Letters, 2016. 590(19): p. 3323-3334.
81. Zhu, S., et al., J Biol Chem, 2016. 291(26): p. 13662-78.
82. Zhang, Y., et al., Frontiers in Microbiology, 2018. 9.
83. Sugase, K., et al., Protein Expression and Purification, 2008. 57(2): p. 108-115.
84. Meyer, A. J., et al., Nature Chemical Biology, 2019. 15(2): p. 196-204.
85. Montalbán-López, M., et al., FEMS Microbiology Reviews, 2017. 41(1): p. 5-18.
86. N/A
87. Segall-Shapiro, T. H., et al., Nature Biotechnology, 2018. 36(4): p. 352-358.
88. Salis, H. M., et al., Nature Biotechnology, 2009. 27(10): p. 946-950.
89. Espah Borujeni, A., et al., Nucleic Acids Research, 2014. 42(4): p. 2646-2659.
90. Clifton, K. P., et al., Journal of Biological Engineering, 2018. 12(1).
91. Michael, J. P., Natural Product Reports, 2007. 24(1): p. 191.
92. Walsh, C. T., et al., ACS Chemical Biology, 2012. 7(3): p. 429-442.
93. Martins, J. and V. Vasconcelos, Marine Drugs, 2015. 13(11): p. 6910-6946.
94. Rosano, G. N. L. and E. A. Ceccarelli, Frontiers in Microbiology, 2014. 5.
95. Himes, P. M., et al., ACS Chemical Biology, 2016. 11(6): p. 1737-1744.
96. Goodman, K. J. and J. T. Brenna, Analytical Chemistry, 1994. 66(8): p. 1294-1301.
97. Zhang, Y., et al., Nature Communications, 2018. 9(1).
98. Li, K., et al., Nature Chemical Biology, 2016. 12(11): p. 973-979.
99. Kallberg, M., et al., Nature Protocols, 2012. 7(8): p. 1511-1522.
100. Bobeica, S. C., et al., eLife, 2019. 8.
101. Jones, S. and J. M. Thornton, Progress in Biophysics and Molecular Biology, 1995. 63(1): p. 31-65.
102. Koehnke, J., et al., Nature Chemical Biology, 2015. 11(8): p. 558-563.
103. Rubin, G. M. and Y. Ding, Journal of Industrial Microbiology & Biotechnology, 2020. 47(9-10): p. 659-674.
104. Panavas, et al., Methods in Molecular Biology. 2009, Humana Press. p. 303-317.
105. Schnell, N., et al., European Journal of Biochemistry, 1992. 204(1): p. 57-68.
106. Schnell, N., et al., Nature, 1988. 333(6170): p. 276-278.
107. Sit, C. S., et al., Accounts of Chemical Research, 2011. 44(4): p. 261-268.
108. Palm, K., et al., Journal of Medicinal Chemistry, 1998. 41(27): p. 5382-5392.
109. Lipinski, C. A., et al., Advanced Drug Delivery Reviews, 2001. 46(1-3): p. 3-26.
110. Kaunietis, A., et al., Nature Communications, 2019. 10(1).
111. Butler, M. S., et al., The Journal of Antibiotics, 2014. 67(9): p. 631-644.
112. Moradi, S. V., et al., Chemical Science, 2016. 7(4): p. 2492-2500.
113. Gavrish, E., et al., Chemistry & Biology, 2014. 21(4): p. 509-518.
114. Mandal, P. K., et al., Journal of Medicinal Chemistry, 2011. 54(10): p. 3549-3563.
115. Kapust, R. B., et al., Biochemical and Biophysical Research Communications, 2002. 294(5): p. 949-955.
116. Eng, C. H., et al., Nucleic Acids Research, 2018. 46(D1): p. D509-D515.
117. Knappe, T. A., et al., Angewandte Chemie International Edition, 2011. 50(37): p. 8714-8717.
118. Kosuri, S. and G. M. Church, Nature Methods, 2014. 11(5): p. 499-507.
119. Klucznik, T., et al., Chem, 2018. 4(3): p. 522-532.
120. Coley, W., Connor, et al., Chemical Science, 2019. 10(2): p. 370-377.
121. Segler, M. H. S., et al., Nature, 2018. 555(7698): p. 604-610.
122. Precord, T. W., et al., ACS Chemical Biology, 2019. 14(9): p. 1981-1989.
123. Das, S. and S. Chakrabarti, Scientific Reports, 2021. 11(1).
124. Tianero, M. D., et al. Proceedings of the National Academy of Sciences, 2016. 113(7): p. 1772-1777.
125. Bennallack, P. R. and J. S. Griffitts, World Journal of Microbiology and Biotechnology, 2017. 33(6).
126. Hudson, G. A., et al., Journal of the American Chemical Society, 2015. 137(51): p. 16012-16015.
127. Sarkar, S., ACS Catalysis, 2020. 10(13): p. 7146-7153.
128. Liu, R., et al., Advanced Science, 2020. 7(17): p. 2001616.
129. Kloosterman, A. M., et al., mSystems, 2020. 5(5).
130. Skinnider, M. A., et al., Proceedings of the National Academy of Sciences, 2016.
113(42): p. E6343-E6351.
131. Kloosterman, A. M., et al., PLOS Biology, 2020. 18(12): p. e3001026.
132. Tietz, J. I., et al., Nature Chemical Biology, 2017. 13(5): p. 470-478.
133. Kloosterman, A. M., et al., Current Opinion in Biotechnology, 2021. 69: p. 60-67.
134. Blin, K., et al., Nucleic Acids Res, 2019. 47(W1): p. W81-W87.
135. Jin, M., et al., Angewandte Chemie International Edition, 2003. 42(25): p. 2898-2901.

### Example 4: Selection for Constrained Peptides that Bind to the SARS-CoV-2 Spike Protein

Peptide secondary metabolites are common in nature and have diverse functions, from antibiotics to cross-kingdom signaling, that have been harnessed as pharmaceuticals. Their amino acid structure simplifies binding to protein targets and they have constraints and chemical modifications that enhance affinity, stability and solubility. A method to design large libraries of modified peptides in Escherichia coli and screen them in vivo to identify those that bind to a target-of-interest was developed in this Example. Constrained peptide scaffolds were produced using modified enzymes gleaned from microbial RiPP (ribosomally synthesized and post-translationally modified peptides) pathways and diversified to build large libraries. RiPP binding to a target protein leads to the intein-catalyzed release of a 6 factor. This circuit was used to drive a selection, which could evaluate 10⁸ variants in a single experiment. This was applied to the discovery of a 1625 Da constrained peptide (AMK-1057) that binds with 990±5 nM affinity to the SARS-CoV-2 Spike receptor binding domain (RBD), a potential therapeutic target.

### INTRODUCTION

Bacteria and fungi secrete modified peptides that can act on eukaryotic cells by binding to cell-surface proteins, inhibiting enzymes or affecting protein-protein interactions [1-3]. They can be produced by large non-ribosomal peptide synthases or encoded by genes and post-translationally modified (RiPPs) [4-8]. As pharmaceuticals, cyclic peptides are approved for the treatment of cancer, inflammation, and infection and increasing numbers are entering all phases of clinical development for diverse indications [9-12]. They have shown promise for blocking viral entry into human cells [13,14]. For example, the FDA-approved HIV therapeutic Enfuvirtide is a 36 amino acid (aa) linear peptide that binds to a transmembrane glycoprotein; however, it suffers from rapid proteolysis, thus requiring twice daily injections [15]. Crosslinking HIV-1 mimetic peptides makes them proteolytically-stable, acid-resistant, and orally bioavailable [16].

Discovering peptides that bind to a therapeutic target requires methods to: (1) create massive pools of chemical diversity, and (2) identify hits in an efficient manner. Synthetic chemistry can be used to create libraries of modified peptides, including cycles and glycosylation, which are screened individually in assays that can be automated [17-24]. Encoding the peptide with its genetic material facilitates the panning for those that bind to a target, for example, using fluorescence activated cell sorting (FACS) [18-20,23, 25-29]. This can be done through yeast display, mRNA-peptide fusions and phage display, which have been used to find modified peptides that are antibiotics or bind human therapeutic targets [26, 29-36]. Cyclization can be performed enzymatically, chemically, or with split inteins, which are naturally occurring proteins that splice two separately-expressed peptides into an excised intein and a product [37,38].

If target binding can be linked to gene expression, this can be used to drive a reporter for screening or a marker that allows cells to survive a selection. The classic example is a two-hybrid system where a “bait” protein fused to DNA-binding domain recruits the “prey” protein fused to an activator that turns on a promoter when bound [39-44]. This can be used to find molecules that disrupt the bait-prey interaction, which has been applied to the discovery of linear peptides that are antivirals or block cancer signaling or progression [40, 45-47]. An E. coli version led to the discovery of a cyclized RiPP μM inhibitor of the p6-UEV protein-protein interaction necessary for HIV budding [41,44]. Protein-protein interactions have also been detected using split inteins where, upon binding, a reporter (epitope, fluorescent protein or a factor) is released, but this has not been applied to molecular discovery [48,49].

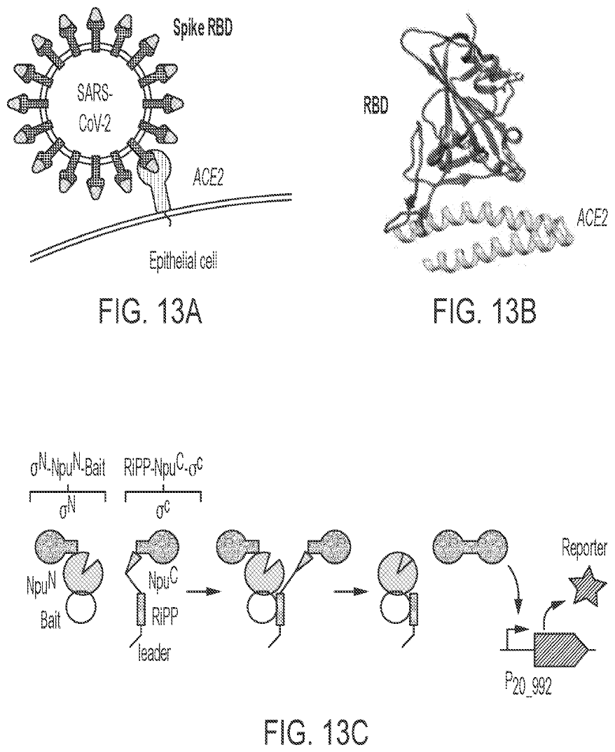

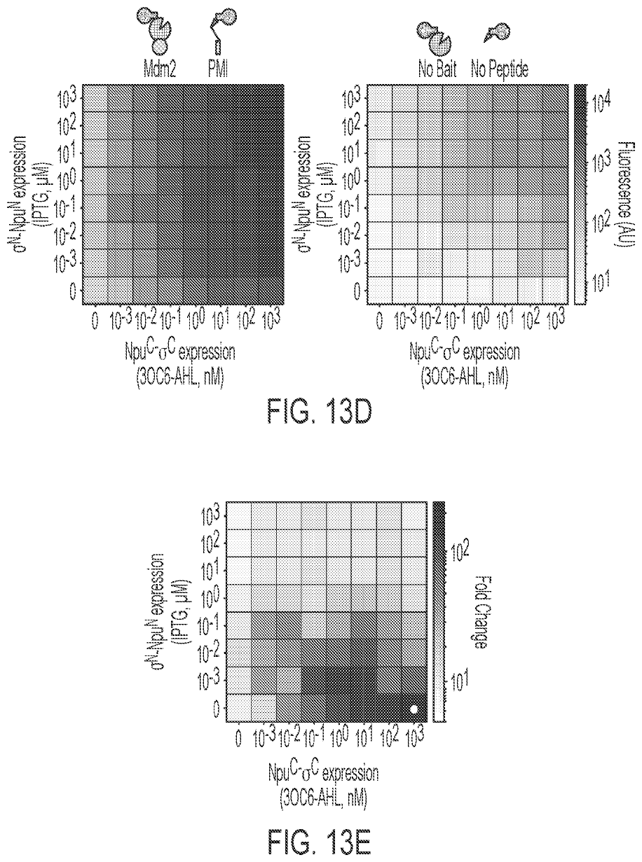

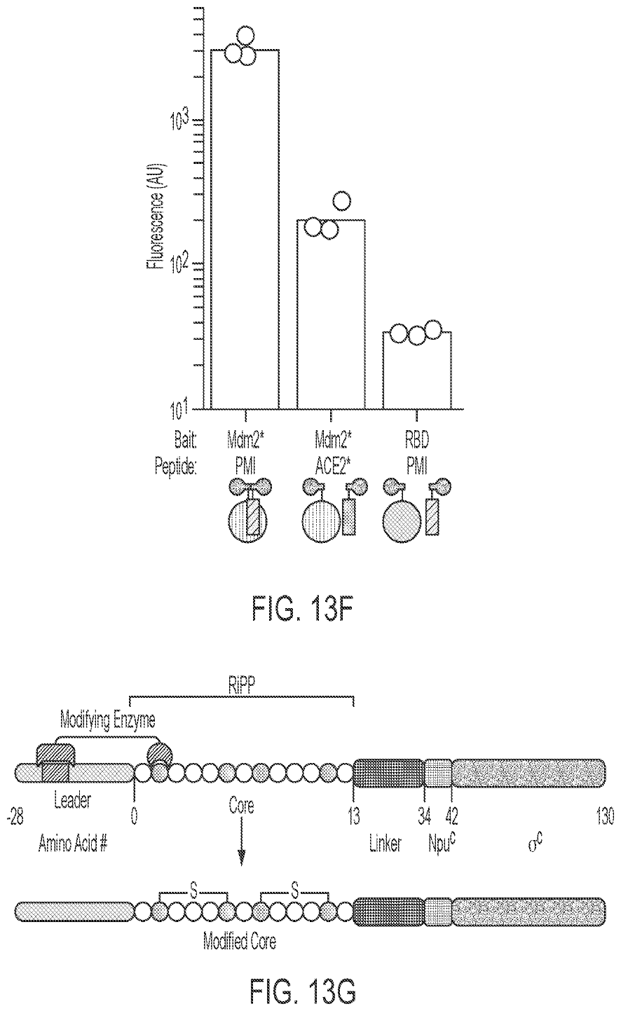

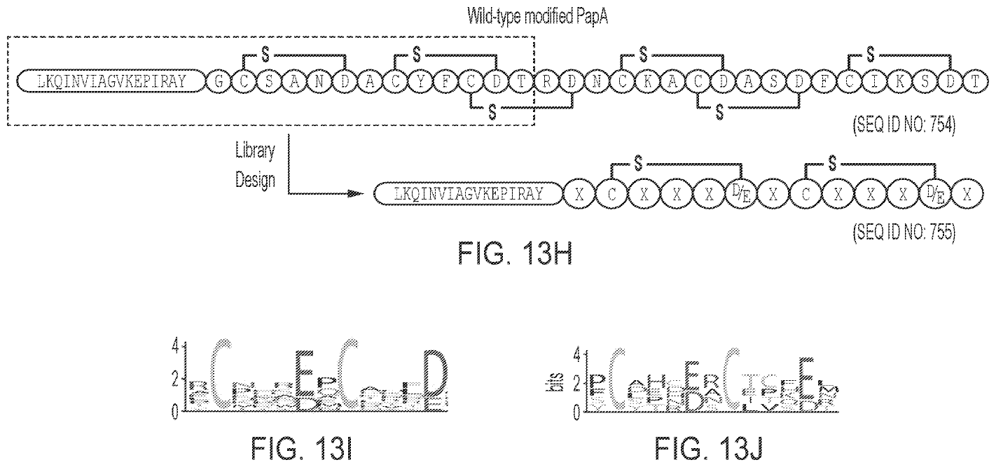

Infection by severe acute respiratory syndrome coronavirus-2 (SARS-CoV-2), the causative agent of COVID-19, is dependent upon cell recognition and entry mediated by the interaction of viral surface glycoprotein (Spike) receptor binding domain (RBD) and host receptor angiotensin-converting enzyme 2 (ACE2) (FIG. 13A, FIG. 13B) [50-53]. The high affinity of RBD to human ACE2 (44.2 nM) has been suggested to contribute to the contagiousness of SARS-CoV-2 [54]. Serum isolated from convalescent coronavirus patients, used for treatment, contains mixtures of antibodies targeting viral epitopes, with Spike protein being the predominant target neutralized [55,56]. Targeted drug discovery efforts from companies including Astra Zeneca/Vanderbilt University, Celltrion, Eli Lilly/AbCellera, Eli Lilly/Junshi, and Tychan have yielded monoclonal antibodies (>1000 aa) specific to Spike protein in various stages of clinical development [57]. So-called nanobodies (˜100 aa) have been evolved in the laboratory to bind to Spike (with 4.5 nM affinity) [58]. Computational protein design was used to develop a “miniprotein” (56 aa), the best of which bound with sub-nM affinity and neutralized virus at 0.15 nM concentration [59]. A biotinylated 23 aa peptide taken from the N-terminal region of ACE2 binds to the Spike RBD with a KD of 1.3 μM [60]. A cyclic peptide based on the SARS-CoV-2 Mpro C-terminal autolytic cleavage site was shown to have an IC50 of 150 μM for the viral protease, another potential therapeutic target [61].

A genetic circuit in E. coli that responds when a modified peptide binds to a single bait protein was developed and used to drive a selection to identify hits that bind to the SARS-CoV-2 Spike RBD. Libraries of modified peptides were produced by artificially combining enzymes from microbial RiPP pathway that introduces thioether-based macrocycles to constrain the peptide (Paenibacillus polymyxa PapB) [62-64] and vary the unmodified core residues. Each candidate RiPP was fused to a C-terminal intein and one half of a split a factor (RiPP-Npu^{C}-σ^{C}) and modified in this context (FIG. 13C). The bait (Spike RBD) was fused to the complementary intein and the other half of the σ factor (σ^{N}-Npu^{N}-Bait). In this system, when the modified RiPP binds to the bait, the complete σ factor is released, binds to a promoter and facilitates the expression of a reporter and/or selection marker. Rounds of positive selection were used to identify RiPPs that bound to the bait RBD in the circuit. From these selections, a 14 aa thioether-cyclized peptide (termed AMK-1057) that binds human-derived SARS-CoV-2 Spike RBD with a K_{D} of 990±5 nM was identified.

### Results

### A Genetic Circuit to Detect Modified Peptide Binding to a Target

The genetic circuit described in this Example converts a binding event into a transcriptional response (e.g., the expression of a reporter protein; FIG. 13C). It is based on two fusion proteins that, upon binding, release a σ factor that recruits RNA polymerase to a promoter, resulting in expression of the reporter downstream of the promoter. Each fusion protein comprises one half of a split intein from Nostoc punctiforme PCC73102 (Npu). The Npu intein was selected because of its stability and rapid splicing kinetics [38]. The σ factor ECF20_992 was chosen based on previous development by the inventors of a split version of it [65,66]. The N-terminus of the fusion protein had the leader peptide that recruits modifying enzymes and the core sequence of the RiPP followed by a flexible 20 aa linker. Successful splicing of full length σ factor resulted in the activation of the P20_992 promoter, thereby turning on the downstream genes (e.g., a reporter gene; FIG. 13C). To develop the sensor, the well-studied interaction between the proteins p53 and Mdm2 was selected as a test case [67]. Specifically, residues 17-124 of Mdm2 (Mdm2*) and a high affinity (K_{D}=0.5 nM) variant of residues 15-29 of p53 (PMI) [68,69] were used as bait and peptide fusions to the σ^{N}-Npu^{N} and Npu^{C}-σ^{C} fragments, respectively. The two genes were placed under the control of the PLuxB and PTac promoters in E. coli Marionette Clone 70 (a derivative of NEB 10β). For characterization, sfGFP was cloned downstream of P20_992 and fluorescence was measured using flow cytometry as a function of the inducers 3O6-AHL and IPTG (FIG. 13D, left panel). The dynamic range between uninduced and fully-induced was 114-fold, and expression varied over orders-of-magnitude while still allowing a response to be observed.

It is important that the expression of the σ^{N}-Npu^{N} and Npu^{C}-σ^{C} fragments, in the absence of bait or peptide, does not induce the circuit. The experiments described above were repeated for these fragments lacking bait or peptide. At maximal expression of the σ^{N}-Npu^{N} and Npu^{C}-σ^{C} fragments (lacking bait or peptide), the output promoter was activated, albeit at 8-fold lower activity than when the bait and peptide were included (FIG. 13D, right panel). This difference was maximized to >200-fold when σ^{N}-Npu^{N} was fully induced and Npu^{C}-σ^{C} fragment was uninduced (FIG. 13D, right panel). To simplify this implementation, the σ^{N}-Npu^{N}-bait protein was placed under the control of a constitutive promoter. The peptide-Npu^{C}-σ^{C} was kept under 3OC6-AHL control so that its intracellular concentration could be varied during rounds of selection to preferentially select for higher-affinity binders.

The inducible range of the sensor was then determined when either the bait or peptide were swapped to disrupt the interaction. When a peptide based on the N-terminal residues 19-56 of ACE2 (ACE2*), which does not bind to the Mdm2* target, was used, the fluorescent output of the circuit dropped 15-fold. Similarly, when the target peptide was swapped to be residues 328-533 of the SARS-CoV-2 Spike protein (RBD) 51, to which PMI does not bind, the output dropped 93-fold (FIG. 13F).

The peptide needs to be able to be modified by RiPP enzymes in the context of its fusion to C-terminal Npu^{C}-σ^{C} (FIG. 13F). The RiPP enzymes were recruited by binding to an N-terminal leader sequence and then modifying the core sequence [4]. Natural RiPPs are proteolytically released from the leader, but for the selections used in this Example, they remain fused because the cognate protease was not co-expressed. Examples have been published where leaders do not interfere with the binding of the core sequence to its target [27,41]. RiPP enzymes often exhibit relaxed substrate specificity for the unmodified core residues [72-74]. In many cases, tags and fusions can be made to the C-terminus without impacting the modification [27,74,75].

A preliminary experiment was performed to ensure that an enzyme of interest could modify a large fraction of core sequences in a library without being impacted by the C-terminal fusion (FIG. 13G). The PapA/PapB peptide/enzyme pair from the Paenibacillus polymyxa freyrasin biosynthetic gene cluster was selected as a test case [63]. PapB introduces a thioether macrocycle between core C and D/E residues and was shown to be tolerant to amino acid diversity at the unmodified residues [63,73]. Based on this system, a simplified core and leader peptide containing two cycles was designed (FIG. 13H). A library was constructed allowing full (NNK) degeneracy at 9 unconserved amino acid positions and D/E at the two macrocyclized positions, resulting in 10¹² theoretical diversity. Nineteen random members from this library were selected, co-expressed with PapB, and evaluated for cyclization by measurement of the mass shift observed using LCMS. Of the original set, 14 were the proper core peptide (1 frame shift) and could be observed by LC-MS, and of these 36% were modified correctly. Thus, a large fraction of a highly diverse library contained the expected modification. The modification did not seem to bias the core amino acid content for the small set analyzed (FIG. 13I, FIG. 13J).

### Selection System for Finding SARS-CoV-2 Spike RBD Binders

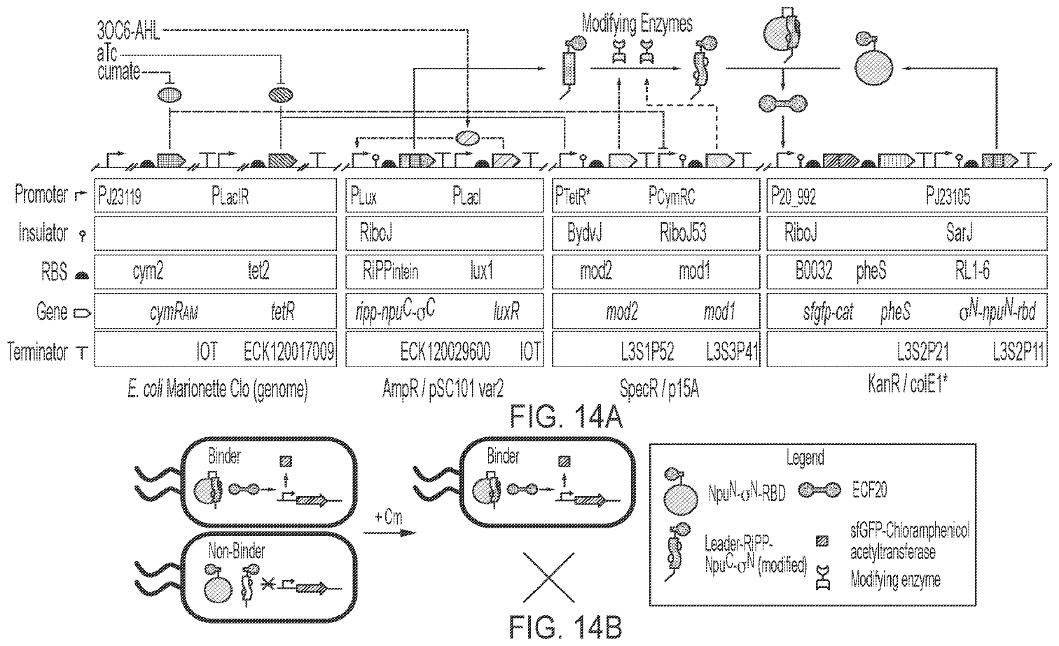

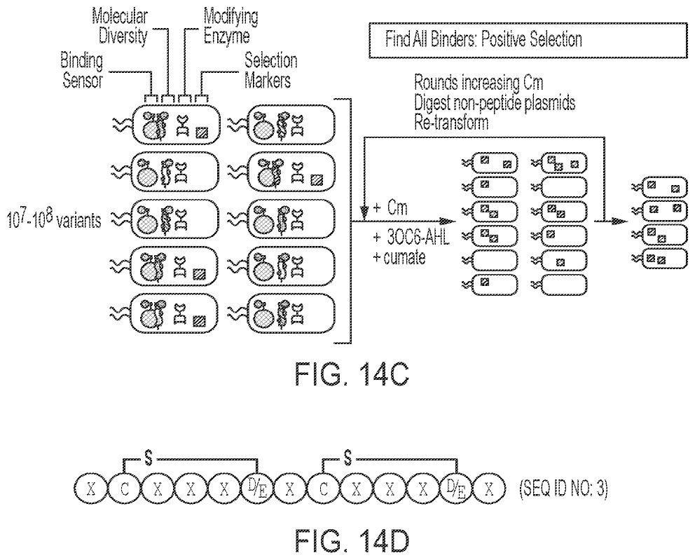

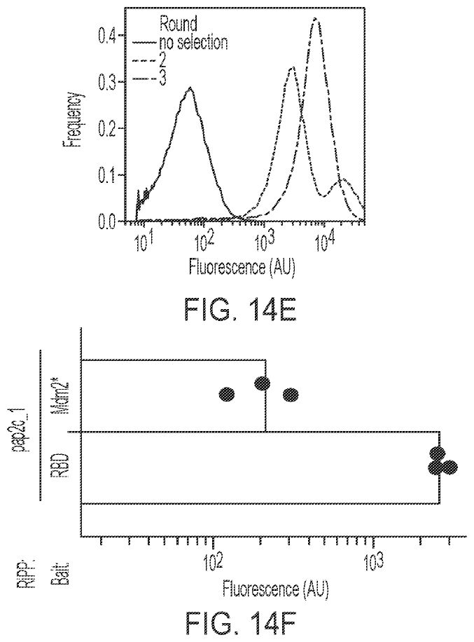

The genetic system used for the selections, involving nine genes, is shown in FIG. 14A. It has been previously demonstrated that the SARS-CoV-2 Spike RBD can be expressed in E. coli and, despite the protein being non-glycosylated, a similar antibody binding profile to that produced from human cells results [76,77]. This domain was used to build σ^{N}-npu^{N}-RBD, which was placed under the control of the weak J23105 constitutive promoter. The peptide library was inserted into the RiPP-npu^{C}-σ^{C} gene and controlled with 3OC6-AHL. The modifying enzyme was placed under the control of the cumate-inducible promoter. When the a factor is released, the P20_992 promoter drives the expression of an operon containing a fluorescent protein selectable marker fusion sfGFP-cat. This enabled positive selection by the addition of chloramphenicol (Cm) to the media.

The libraries of modified peptides were constructed using oligo synthesis with NNK codons at the varied residues and cloned into a low copy pSC101 plasmid. The library was transformed using electrocompetence, which was found to limit the library size to 10⁸ per transformation. Then, multiple rounds of positive selection were performed. The details for each library are described further below. When a RiPP binds the target, expression of Cat is increased, thereby conferring chloramphenicol resistance to the host cell (FIG. 14B). Over rounds of positive selection, increased stringency can be applied by increasing the concentration of Cm or decreasing peptide induction with 3OC6-AHL (FIG. 14C).

### Library Design and Selection

The library was based on the simplified PapB-modified core structure shown in FIG. 14D which produces 13 aa cyclized peptides. After transformation, rounds of positive selection were performed, after which the surviving plasmids were isolated and retransformed after each round (FIG. 14C). Cells were grown overnight in increasing concentrations of Cm: Round 1 (300 μM), Round 2 (800 μM) and Round 3 (1200 μM). sfGFP expression was measured after each round using flow cytometry, showing a continuous increase in the fluorescence after each round of selection (FIG. 14E). Notably, when using 300 μM Cm for the initial selection, two peaks were observed: one lower (˜2,000 AU) and much higher (˜10,000 AU). This higher peak was attributed to escape mutants from the selection plasmid breaking. To eliminate escapes from round to round, non-peptide plasmid was digested and re-transformed into the expression strain, eliminating the peak corresponding to escapes (FIG. 14E). All selection rounds were then analyzed using next-generation sequencing (NGS). The number of unique RiPP sequences decreased after each round, indicating enrichment: 139,320 (Round 1), 88,229 (Round 2) and 63,344 (Round 3). The abundance of each sequence was calculated and 32 were found to represent >1% of the population each after Round 3. These were further reduced to 20 by only considering those that showed consistent enrichment from Round 1 to 2 and from Round 2 to 3.

The 20 hits from this library were codon optimized, re-synthesized and cloned into the RiPP-npu^{C}-σ^{C} plasmid and re-assessed in freshly transformed cells. Testing of newly synthesized constructs was intended to eliminate any cheater behavior that may have arisen throughout the selection process. These constructs were transformed into selection strains containing cognate modifying enzymes and either Spike RBD or Mdm2* as bait, with the latter intended to measure off-target binding. The circuit output was measured using flow cytometry under the same growth conditions and inducer concentrations used for the selections. The core sequence VCKYGEWCEIVEI (SEQ ID NO: 24) demonstrated a strong transcriptional output and 14-fold specificity for the Spike RBD as bait over Mdm2* (FIG. 14F).

### AMK-1057 Binds Human Cell-Derived SARS-CoV-2 RBD

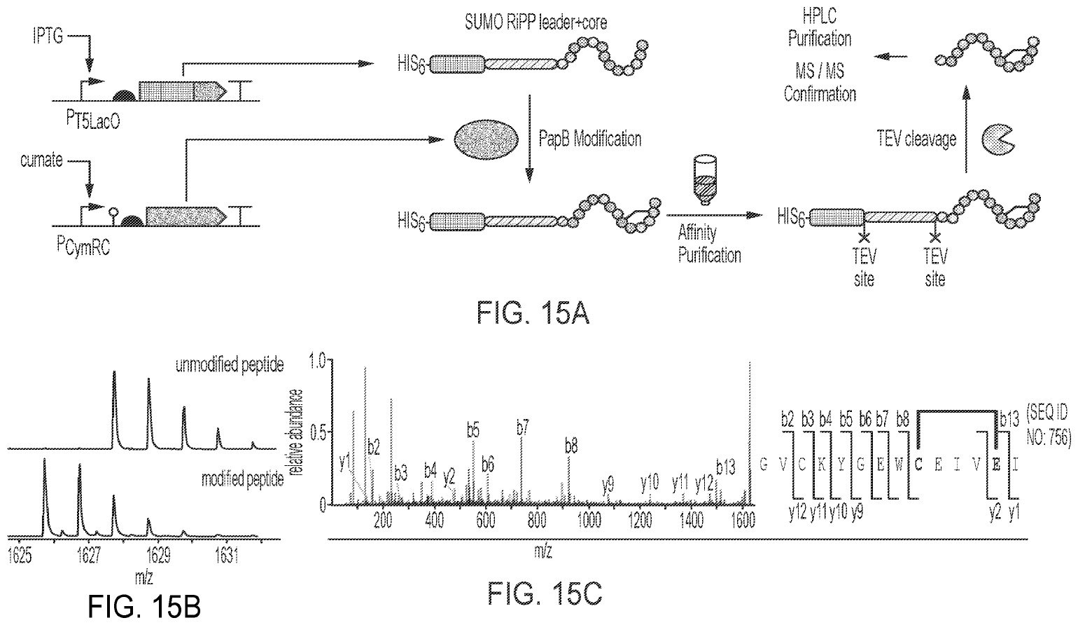

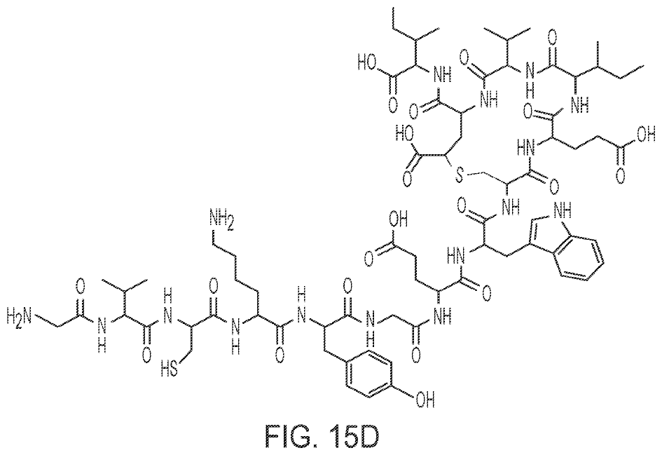

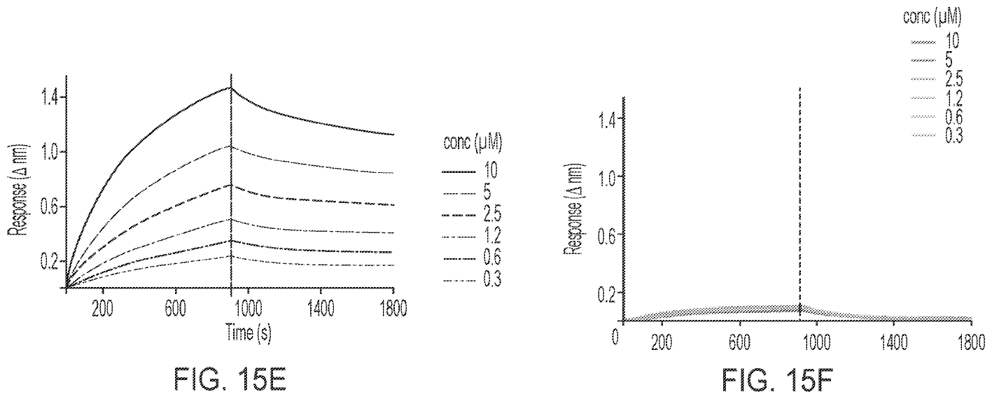

The core sequence VCKYGEWCEIVEI (SEQ ID NO: 24) underwent liter-scale production, cleavage and purification (FIG. 15A). The peptide gene was cloned as a C-terminal fusion to a hexa-histidine-Small Ubiquitin-like Modifier (SUMO) tag under control of the PT5LacO promoter and strong ribosome binding site (no longer in the context of the Npu^{C}-σ^{C} fragment). SUMO is a small (12 kDa) tag often used in heterologous protein purification that has been found to be effective in stabilizing RiPP peptide expression while not interfering with modifying enzyme activity [78]. A Tobacco etch virus (TEV) cleavage site was added between the leader and core regions for downstream processing. This left a glycine on the N-terminus of the pap2c_1 peptide, thus producing a 14 aa peptide that, in its modified form, was named AMK-1057. Note there is also a TEV site upstream of the leader sequence, liberating it from SUMO as well so that it can be used as a control.

Co-expression of this peptide fusion with PapB in E. coli Marionette X (NEB Express derivative) cells followed by Ni-NTA affinity purification yielded tagged and modified pap2c_1. A peak corresponding to unmodified peptide was also detected. Dialysis of Ni-NTA purified peptide, TEV cleavage, solid phase extraction (SPE) and semiprep HPLC purification led to the isolation of three peptides: leader (yield: 200 μg/L), unmodified core (640 μg/L) and modified core (360 μg/L).

High resolution LCMS analysis of both modified (expected m/z: 1625.7338; observed m/z: 1625.7332) and unmodified (expected m/z: 1627.7494; observed m/z: 1627.7484) peptide showed a mass shift corresponding to formation of a single cycle, despite the library being based on a two-cycle scaffold (FIG. 15B). The macrocycle found in AMK-1057 is formed through the covalent linkage of a side chain cysteine sulfur atom to the CP on the downstream glutamate residue, a linkage that is stable to standard collision-induced dissociation conditions [63]. This property was used to annotate the macrocycle placement via high-resolution tandem MS (HR-MS/MS) and hypothetical structure enumeration and evaluation (FIG. 15C) [79]. Fragmentation analysis indicated that the macrocycle forms at the C-terminal end of AMK-1057, between C9 and E13 (FIG. 15D).

In vitro binding experiments were then performed using Expi293F human cell-derived and purified RBD. Bio-layer interferometry (BLI) was used to measure the affinity of AMK-1057 to Spike RBD as 990±5 nM (FIG. 15E). Neither the purified unmodified core peptide (FIG. 15F) nor the leader sequence (not shown) showed any binding to the target.

### DISCUSSION

This Example demonstrates a technique to capture modified peptides that bind to a single target protein. There are several advantages over a two-hybrid screen, including that the binding target does not have to be known (or be a protein) or able to be expressed in a heterologous host, and hits will not be discovered against the “wrong” target (in this case, to human ACE2). As a relevant example of the importance of this capability, clinically relevant betacoronaviruses to date share a common Spike protein for host recognition, but the host receptor is not known a priori [50]. This allows for the search for binders to begin before their cellular targets have been fully elucidated. The libraries provided in this Example are based on natural products built with RiPP enzymes, a family that has been rapidly growing and for which there are many interesting chemical conversions, including halogenation, backbone N-methylation, and β-amino acid formation [80-82]. Larger biologics, such as antibodies, can have problems with stability and are limited in possible modes of delivery [59]. In contrast, cyclic peptides can exhibit improved stability, be cell-permeable thereby enabling access to intracellular antiviral targets, and be suitable for administration via inhalation [83-86].

Using this approach, a small peptide binder to SARS-Cov Spike RBD was identified. At ˜1600 Da, AMK-1057 is a size that is common for peptide secondary metabolites and approaches the threshold for the commonly used definition of a small molecule (˜900 Da) [9]. At <1 μM binding, AMK-1057 is in the higher range of natural RiPPs binding to their target (e.g., lassomycin at 0.41 μM, microcin J25 at 2 μM) and some peptidic drugs (e.g., vancomycin at ˜1 μM) [87-89]. As the first hit to emerge from a selection, it is ripe for further optimization through additional diversification and medicinal chemistry. This work represents a critical initial step of drug discovery. Putative therapeutics targeting viral fusion need to progressively tested in assays for the blockage of viral entry into cell lines [90-93], followed by animal models [92,93]. A human organ-chip has also been developed to screen repurposed drug compound collections that inhibit viral pseudoparticles expressing SARS-CoV-2 Spike from infecting human lung epithelial cells [94]. Combining molecular diversity creation using the method provided herein with a selection circuit in the same cell enables massive libraries to be evaluated to populate these pharmaceutical discovery pipelines with binders to a target-of-interest with minimal biochemical information.

### Materials and Methods

### Strains, Media, and Chemicals.

E. coli NEB 10-beta (C3019I, New England BioLabs, Ipswich, Mass., USA) was used for all routine cloning. E. coli Marionette-Clo 70 was used for all selection experiments. E. coli Marionette-X, a Marionette-compatible derivative of NEB Express (C2523I, New England BioLabs, Ipswich, Mass., USA) was used for large-scale peptide expression experiments. TB (T0311, Teknova, Hollister, Calif., USA) supplemented with 0.4% glycerol (BDH1172-4LP, VWR, OH, USA) was used for peptide expression and modification. 2xYT liquid media (B244020, BD, Franklin Lakes, N.J., USA) and 2xYT+2% agar (B214010, BD, Franklin Lakes, N.J., USA) plates were used for routine cloning and strain maintenance. SOB liquid media (S0210, Teknova, Hollister, Calif., USA) was used for making competent cells. SOC liquid media (B9020S, New England BioLabs, Ipswich, Mass., USA) was used for outgrowth. Unless noted otherwise, cells were induced with the following chemicals: cuminic acid (268402, Millipore Sigma, Saint Louis, Mo., USA) added as 1000× stock (200 mM) in EtOH or DMSO; 3-oxohexanoyl-homoserine lactone (3OC6-AHL) (K3007, Millipore Sigma, Saint Louis, Mo., USA) added as a 1000× stock (1 mM) in DMSO; anhydrotetracycline (aTc) (37919, Millipore Sigma, Saint Louis, Mo., USA) added as a 1000× stock (100 PM) in DMSO; isopropyl β-D-1-thiogalactopyranoside (IPTG) (I2481C, Gold Biotechnology, Saint Louis, Mo., USA) added as 1000× stock (1 M) in water. Cells were selected with the following antibiotics: carbenicillin (carb, C-103-5, Gold Biotechnology, Saint Louis, Mo., USA) added as 1000× stock (100 mg/mL in H2O); kanamycin (kan, K-120-10, Gold Biotechnology, Saint Louis, Mo., USA) as 1000× stock (50 mg/mL in H2O); spectinomycin (spec, S-140-5, Gold Biotechnology, Saint Louis, Mo., USA); and chloramphenicol (Cm, C-105-5, Gold Biotechnology, Saint Louis, Mo., USA). Liquid chromatography was performed with Optima Acetonitrile (A996-4, Thermo Fisher Scientific, MA, USA) and water (Milli-Q Advantage A10, Millipore Sigma, Saint Louis, Mo., USA) supplemented with LCMS Grade Formic Acid (85178, Thermo Fisher Scientific). The following solvents/chemicals were also used: Ethanol (V1001, Decon Labs, King of Prussia, Pa., USA), Methanol (3016-16, Avantor, Center Valley, Pa., USA), Ammonium bicarbonate (A6141 Millipore Sigma, Saint Louis, Mo., USA), dimethyl sulfoxide (DMSO) (32434, Alfa Aesar, Ward Hill, Mass., USA), Imidazole (IX0005, Millipore Sigma, Saint Louis, Mo., USA), sodium chloride (X190, VWR, OH, USA), sodium phosphate monobasic monohydrate (20233, USB Corporation, Cleveland, Ohio, USA), sodium phosphate dibasic anhydrous (204855000, Acros, N.J., USA), guanidine hydrochloride (50950, Millipore Sigma, Saint Louis, Mo., USA), tris (75825, Affymetrix, Cleveland, Ohio, USA), TCEP (51805-45-9, Gold Biotechnology, Saint Louis, Mo., USA), and EDTA (0.5M stock, 15694, USB Corporation, Cleveland, Ohio, USA). DNA oligos and gBlocks were ordered from Integrated DNA Technologies (IDT) (San Francisco, Calif., USA).

### Plasmids and Genes.

Plasmids pTHSS-1282 and pAMK-267 were constructed from the parental pTHSS-1193 backbone, which has a pSC101 origin variant (var 2) and ampicillin resistance [95]. Plasmids pTHSS-1282 and pAMK-267 contain a flexible linker sequence (GSSRGGKGGPGGRGGVGGGGGIGG (SEQ ID NO: 113)) between the peptide/sfGFP and NpuC regions. Plasmids pAMK-925, pTHSS-2132, pAMK-866, and pAMK-870, were constructed from the parental pTHSS-1458 backbone, which has a colE1* origin variant and a kanamycin resistance marker [95]. All plasmids carrying modifying enzymes were constructed from the parental pEG01_189 backbone and contain a p15A origin of replication and spectinomycin resistance [78]. The parental backbone pTHSS-2012, which has a p15a origin and spectinomycin resistance was used for additional cloning experiments [95]. The plasmid pTHSS-1282 that contains the P20_992 promoter expressing sfGFP was constructed from pTHSS-1193. The plasmids pAMK-926 and pTHSS-2137 that contain the PLux promoter expressing Npu^{C}-σ^{C} and PMI-Npu^{C}-σ^{C}, respectively, were constructed from pTHSS-2012. The plasmids pAMK-925 and pTHSS-2132 that contain the PTac promoter expressing σ^{N}-Npu^{N} and residues 17-124 of Mdm2 (Mdm2*)-σ^{N}-Npu^{N}, respectively, were constructed from pTHSS-1458. The plasmid pAMK-870 that contains the constitutive PJ23105 promoter expressing Mdm2*-σ^{N}-Npu^{N} and the P20_992 promoter expressing CAT-sfGFP was constructed from pTHSS-1458. The plasmid pAMK-866 that contains the constitutive PJ23105 promoter expressing 328-533 of the SARS-CoV-2 Spike protein (RBD)-σ^{N}-Npu^{N} and the P20_992 promoter expressing CAT-sfGFP was constructed from pTHSS-1458. The peptide cloning plasmid pAMK-267, constructed from pTHSS-1193, contains the PLux promoter upstream of an RBS-His tag-SapI-sfGFP-SapI-Npu^{C}-σ^{C} where the sfGFP gene can be replaced by a peptide gene through Type IIs assembly methods using the enzyme SapI (NEB). The RBS from pAMK-267 was chosen from a library of RBS variants upstream of a His tag-PMI-Npu^{C}-σ^{C} that was tuned for co-expression with constructs containing the PJ23105 promoter expressing Mdm2*-σ^{N}-Npu^{N}. The N-terminal His tag in pAMK-267 was left in place to provide a constant 11 aa for consistent levels of expression between different peptide sequences. The plasmid pAMK-670 that contains the PLux promoter expressing PMI-Npu^{C}-σ^{C} was constructed from pAMK-267. The plasmid pAMK-857 that contains the PLux promoter expressing N-terminal residues 19-56 of ACE2 (ACE2*)-Npu^{C}-σ^{C} was constructed from pAMK-267. The pTHSS-1193 and pTHSS-1458 backbones have origin variants that alter their copy numbers, making them approximately equivalent to a p15a backbone. Genes encoding Npu intein, PMI, Mdm2*, ACE2*, and RBD were synthesized as gBlocks. The ECF20_992 gene was sourced from a previous publication [65].

### Cytometry Analysis.

Fluorescence characterization was performed on a BD LSR Fortessa flow cytometer with HTS attachment (BD, Franklin Lakes, N.J., USA). Samples were prepared by diluting overnight cultures 1:400 by adding 0.5 μl of cell culture into 200 μl of PBS containing 1 mg/mL Kan. All samples were run in standard mode at a flow rate of 0.5 μl/s. Fluorescence measurements were made using the blue (488 nm) laser and all data was derived from the FITC-A channel (PMT voltage of 400 V). The FSC and SSC voltages were 650 V and 270 V, respectively. At least 30,000 events were collected for each sample and the Cytoflow Python package was used for downstream analysis. Gating was completed by fitting a 2D Gaussian function to the FSC and SSC distributions and excluding all events greater than three standard deviations from the mean. When presented, the median value is used.

### Evaluation of the Split-Intein σ Factor Circuit.

Strains of E. coli Marionette Clo harboring a combination of plasmids pTHSS-1282, pTHSS-2132, and pTHSS-2137 or pTHSS-1282, pAMK-925, and pAMK-926 were used for assessing intein splicing with or without PMI-Mdm2* induced association, respectively. Strains were grown in 1 mL of LB+ antibiotics for 20 hr in a deep well 96-well plate (1896-2000, USA Scientific, FL, USA) at 30° C., 900 rpm in an Infors HT Multitron Pro (Infors USA, MD, USA). Cultures were then diluted 1:100 into fresh 1 mL of LB+ antibiotics and serial 1:10 dilutions of inducers (IPTG, 10⁻³-10³ μM; 3O6-AHL, 10⁻³-10³ nM) for 20 hr in a deep well 96-well plate at 30° C., 900 rpm in the Multitron Pro. 0.5 μl of saturated cell culture were then diluted into 200 μl of PBS containing 1 mg/mL kan for cytometry analysis.

### Two-Hybrid Assay for RBD/Mdm2* Association.

To assay for protein-protein mediated splicing the following plasmid combinations were transformed into E. coli Marionette Clo and fluorescence was measured via cytometry: pAMK-866/pAMK-670 (RBD/PMI); pAMK-866/pAMK-857 (RBD/ACE2*); pAMK-870/pAMK-670 (Mdm2*/PMI); pAMK-870/pAMK-857 (Mdm2*/ACE2*). Strains were grown in 1 mL of LB+ antibiotics for 20 hr in a deep well 96-well plate at 30° C., 900 rpm in a Multitron Pro. Cultures were then diluted 1:100 into fresh 1 mL of LB+ antibiotics+1 μM 3O6-AHL (full induction of peptide plasmid) for 20 hr in a deep well 96-well plate at 30° C., 900 rpm in the Multitron Pro. 0.5 μl of saturated cell culture were then diluted into 200 μl of PBS containing 1 mg/mL Kan for cytometry analysis.

### Library Generation.

The Pap library was designed with diversity at the ends and middle of the peptide and included either glutamate or aspartate as a cyclization partner, for a final sequence design of “XCXXX[D/E]XCXXX[D/E]X (SEQ ID NO: 114)”. Using the degenerate nucleotide sequences “NNK” to encode any amino acid and “GAW” for aspartate or glutamate, a library of 10¹² peptides encoded by 10¹⁴ unique codon sequences was generated. The library of plasmids lbAMK-103, which contains the PLux promoter expressing the Pap library-Npu^{C}-σ^{C} was constructed from pAMK-267. The pap library was amplified from pEG03_283 using degenerate oligonucleotides oAMK-915/916 (IDT). Gel purification was used to isolate the 124 bp amplicon, which was then cloned into pAMK-267 using the type IIS restriction enzyme SapI (NEB).

Linear insert and plasmid were mixed at a 1:1 molar ratio (200 fmol each) along with 10 μl 1×DNA ligase buffer, 2 μl T4 DNA ligase (HC) (20 U/μl) (M1794, Promega, Madison, Wis., USA) and 4 μl SapI in 100 μl total volume. Reactions were cycled 25 times for 2 min at 37° C. and 5 min at 16° C. then incubated for 30 min at 50° C., 30 min at 37° C., and 10 min at 80° C. in a DNA Engine cycler (Bio-Rad, Hercules, Calif., USA). An additional 2 μl SapI was then added, and the assembly was incubated for 1 h at 37° C. Assemblies were then purified using Zymo Spin I columns (Zymo Research, Irvine, Calif., USA). Library assemblies were initially transformed into electrocompetent NEB 10βE. coli (C3020K, NEB, Ipswich, Mass., USA). 1.5×10⁷ colony forming units (CFU)/mL were observed for lbAMK-103. Total transformants were estimated by colony counting after 10⁷-fold dilution and plating of liquid outgrowths on selective media.

### Calculation of the Modified Fraction of the Library.

The initial, unselected papA library was transformed and plated to resolve individual colonies. A set of 19 random colonies were picked and sequenced via colony PCR. Of the 19 sequenced colonies, 18 were properly assembled. These 18 library members were then assessed for post-translational modification via LCMS. The 9 unmodified and 5 modified library sequences were then aligned and WebLogos generated (weblogo.berkeley.edu/logo.cgi) with default parameters, except without small sample correction.

Selection of Pap Library lbAMK-103.

Assembled library of plasmids lbAMK-103 was transformed into an electrocompetent Marionette Clo strain harboring the PapB modifying enzyme plasmid, pEG06_044, and the selection plasmid, pAMK-866 (all non-assembly transformation steps were >1×10⁸ efficiency). A 1 mL of liquid outgrowth of library transformants was diluted 1:50 in TB+Carb/Kan/Spec+1 μM 3OC6-AHL and 100 μM cumate to induce peptide+modifying enzyme, and grown at 30° C., 250 r.p.m. for 20 h. For the first round of selection, cultures were then diluted 1:100 in TB Carb/Kan/Spec+1 μM 3OC6-AHL and 100 μM cumate+300 μM Cm and grown at 30° C., 250 r.p.m. for at least 20 h (until cultures were saturated). A 0.5 μL aliquot of was taken for cytometry analysis and 2 mL of culture was also taken to harvest plasmid. A 5 μL sample of purified plasmid was stored for NGS analysis and the rest was digested with 1 μL SapI (NEB) for 1 hour at 37° C. to remove the background pEG06_044/pAMK-866 plasmid. The selected lbAMK-103 plasmid was then re-transformed into the strain of electrocompetent E. coli Marionette Clo strain harboring the PapB modifying enzyme, pEG06_046, and the selection plasmid, pAMK-866. The selection process was repeated once more with a Cm concentration of 800 μM and then once more with a Cm concentration of 1200 μM.

### Ngs Analysis.

Library construction for NGS was performed using the protocol for “KAPA Hyper Prep Kits with PCR Library Amplification/Illumina series” (KK8504, Roche). First, miniprepped library plasmids were amplified with Q5 polymerase (#M0492L, New England BioLabs, Ipswich, Mass., USA) with the primers oAMK-946/947 (Pap library) and oAMK-997/998 (Tgn/Lyn library). A 150 bp band was isolated via gel extraction. Indexed adapters were ligated and reamplified with 10 cycles of PCR. Gel extraction was then used to isolate the resultant 260 bp PCR product. Sample concentrations were calculated using a BioAnalyzer on a High Sense DNA chip (5067-4626, Agilent). Samples were diluted to 2 nM, denatured, and further diluted to 10 μM, with a 10% phiX spike in. Samples were run on a HiSeq 2500 using HiSeq v2 reagents for Paired End Clustering and a 200 cycle SBS kit (PE-402-4002 and FC-402-4021, Illumina). Forward and reverse reads were both 110 cycles, with an 8-cycle single index read. Base-calling and demultiplexing were performed using the bcl2fastq software (Illumina) with default settings. After basecalling and indexing, sequences were identified and aligned using the leader sequence and then binned by sequence.

Validation of sequences from NGS. Hit peptides from NGS were resynthesized as gBlocks (IDT). These gBlocks were used as template for PCR to introduce SapI restriction sites compatible for re-cloning into the pAMK-267 library backbone. Newly reconstructed library members were transformed into Marionette-Clo cells containing modifying enzyme and selection plasmids and were then plated on media containing Carb/Kan/Spec. Individual transformants were then cultured in TB+Carb/Kan/Spec in a deep well 96-well plate (1896-2000, USA Scientific, FL, USA) and incubated overnight (Multitron Pro, 30° C., 900 rpm). These cultures were then subcultured 1:100 in TB+Carb/Kan/Spec either fully induced (1 μM 3OC6-AHL, and 100 μM cumate) or uninduced and incubated for 20 hr (Multitron Pro, 30° C., 900 rpm) before taking 0.5 μL for standard flow cytometry analysis.

### Peptide Purification.

Potential peptide hit gBlocks were cloned into the peptide expression plasmid, pEG03-119 78 using their flanking SapI restriction sites. The peptide and modifying enzyme plasmids were co-transformed into E. coli Marionette-X, streaked onto 2xYT agar with carb/spec and incubated at 30° C. overnight. Individual colonies were used to inoculate 20 mL of LB in a 125 mL shake flask and incubated overnight at 30° C. and 250 rpm in an Innova44 (Eppendorf, N.Y., USA). A 5 mL aliquot of overnight starter culture was diluted in 500 mL total volume TB with carb/spec in Fernbach flasks and grown at 30° C. and 250 rpm until reaching OD600 of 0.8-1.0, at which point 1 mM IPTG and 200 μM cumate were added. Induced cultures were grown for a further 20 h at 30° C. and 250 rpm and then centrifuged (4,000 g, 4° C., 10 min) in a Sorvall RC 6+ centrifuge (Thermo Fisher Scientific, MA, USA). Pellets were resuspended in 30 mL lysis buffer (5 M guanidinium hydrochloride, 300 mM NaCl, 10 mM imidazole, 50 mM sodium phosphate, pH 7.5), and freeze-thawed twice (frozen in −80° C. freezer; thawed in innova44 incubator at 30° C., 250 rpm). Cell lysates were centrifuged (Eppendorf 5424, 20,000 g, 18° C., 45 min) in a Sorvall RC 6+ centrifuge (Thermo Fisher Scientific, MA, USA) and the peptides affinity purified via gravity-flow using 3 mL resin-bed volume of Ni-NTA agarose resin (88223, Thermo Fisher Scientific, MA, USA), following manufacturer instructions, using 2 resin-bed volumes water and lysis buffer for column equilibration, 4 resin-bed volumes of wash buffer (5 M guanidinium hydrochloride, 300 mM NaCl, 25 mM imidazole, 50 mM sodium phosphate, pH 7.5), 4 resin-bed volumes of elution buffer (5 M guanidinium hydrochloride, 300 mM NaCl, 250 mM imidazole, 50 mM sodium phosphate, pH 7.5). Eluate from Ni-NTA purification was then subjected to solid-phase extraction (SPE) using Strata-XL 500 mg tubes (8B-S043-HCH, Phenomenex, CA, USA). The solid phase was first conditioned with 4 bed volumes of methanol and then water. Eluate was then loaded, washed with 8 bed volumes of 10 mM NH₄CO₃, and eluted with 8 bed volumes of 1:1 acetonitrile:aqueous 10 mM NH₄CO₃. Solvent was removed via lyophilization at −80 C for 24-48 hours. To cleave the SUMO and leader peptide from the core, the extracted peptide was resuspended in 20 mL TE buffer and 100 μl of 20 mg/mL TEV protease and incubated overnight at room temperature with slow orbital shaking. The cleaved peptides were then desalted using a Strata-X PRO 500 mg SPE tubes (8B-S536-HCH, Phenomenex, CA, USA). The solid phase was first conditioned with 4 bed volumes of methanol and then water. Eluate was then loaded, washed with 8 bed volumes of 10 mM NH₄CO₃, and eluted with 8 bed volumes of 1:1 acetonitrile:aqueous 10 mM NH₄CO₃. Solvent was removed via lyophilization at −80 C for 24-48 hours. After solvent removal, a 5 mL aliquot of the mixture resuspended in 10:90 acetonitrile:water was injected into a Agilent Technologies 1260 Infinity system HPLC (Agilent Technologies, Santa Clara, Calif.) and separated using a 150 mm×10 cm Aeris PEPTIDE XB-C18 column (100 Å, 5 μm) at a flow rate of 2 mL/min. Separation was carried out with a gradient program, with 0.1% formic acid as solvent A and acetonitrile with 0.1% formic acid as solvent B. The % B was held at 25% for 3 minutes, then increased to 50% over the next 17 minutes. The eluent was passed through a diode array detector (DAD) and absorbance at 270 nm was recorded. Detected peaks were collected using an Agilent G1364B Fraction Collector and again solvent was removed via lyophilization at −80 C for 24-48 hours. Samples were resuspended in 1 mL of 1:1 acetonitrile:aqueous 10 mM NH₄CO₃ in pre-weighed 2 mL microcentrifuge tubes (Eppendorf) and solvent was removed via lyophilization at −80 C for 24-48 hours. Yields were measured by comparing mass of empty tubes to tubes containing lyophilized powder.

### Liquid Chromatography/Mass Spectrometry.

All chromatography was performed using the mobile phases ACN (acetonitrile supplemented with 0.1% formic acid and 0.1% water) and water (supplemented with 0.1% formic acid). The “QTOF” was an Agilent 1260 Infinity II liquid chromatograph with binary pump configured in low-dwell volume mode and column oven set to 40° C., coupled to an Agilent 6545 QTOF mass spectrometer equipped with an Agilent electrospray ionization (ESI) source. ESI source parameters are 350° C. gas temperature, 12 L/min gas flow, 30 psig nebulizer pressure, 350° C. sheath gas temperature, 8 L/min sheath gas flow, 3000 V capillary voltage, 1000 V nozzle voltage, 135 V fragmentor voltage, 15 V skimmer voltage, 600 V Oct 1 RF Vpp; the mass spectrometer was run in MS mode with reference mass enabled and tuned in positive mode with standard mass range (3200 m/z) and 2 GHz extended dynamic range. QTOF analysis was performed with a Phenomenex Aeris PEPTIDE XB-C18 2.6 μm 50 mm×2.1 mm column. The flow rate was set at 0.5 mL/min and 5 μl sample was injected. The gradient used was 20% ACN for 0.5 min, 20% to 55% ACN over 5.5 min, 55% to 90% ACN over 0.5 minutes, 90% ACN for 1.5 min, with 0.8 min re-equilibration. Accurate mass predictions of peptides were generated using the online resource, ChemCalc [96].

### Bio-Layer Interferometry.

Assays were performed on an Octet Red (ForteBio) instrument at 30° C. with shaking at 1,000 rpm. Ni-NTA biosensors (18-5101, ForteBio, Bohemia, N.Y., USA) were hydrated in 1× kinetics buffer (diluted from 10× buffer; 18-5032, ForteBio, Bohemia, N.Y., USA) for 30 min before the measurement. Expi293F human cell-derived and purified SARS-CoV-2 RBD (RBD296-531) was loaded at 10-20 μg/mL in 1× Kinetics Buffer for 300 s prior to baseline equilibration for 180 s in 1× kinetics buffer. Association reactions of the peptide to RBD296-531 were carried out in 1× kinetics buffer at various concentrations in a two-fold dilution series from 80 mM to 1.25 mM was carried out for 900 s. Then dissociation reactions were observed for 900 s. Response data were generated using ForteBio data analysis software.

### REFERENCES FOR EXAMPLE 4

1 Zhang, H. et al. Eur. J. Med. Chem. 156, 847-860, doi:10.1016/j.ejmech.2018.07.023 (2018).
2 Okino, T., et al., Tetrahedron 51, 10679-10686, doi:10.1016/0040-4020(95) 00645-0 (1995).
3 Schreiber, S. L. & Crabtree, G. R. Immunol. Today 13, 136-142, doi:10.1016/0167-5699(92) 90111-J (1992).
4 Arnison, P. G. et al. Nat. Prod. Rep. 30, 108-160, doi:10.1039/C2NP20085F (2013).
5 Fischbach, M. A. & Walsh, C. T. Chem. Rev. 106, 3468-3496, doi:10.1021/cr0503097 (2006).
6 Whitty, A. et al. Drug Discov. Today 21, 712-717, doi:10.1016/j.drudis.2016.02.005 (2016).
7 Villar, E. A. et al. Nat. Chem. Biol. 10, 723-731, doi:10.1038/nchembio.1584 (2014).
8 Ermert, P., et al., Methods Mol. Biol. 2001, 147-202, doi:10.1007/978-1-4939-9504-2_9 (2019).
9 Luther, A., et al., Curr. Opin. Chem. Biol. 38, 45-51, doi:10.1016/j.cbpa.2017.02.004 (2017).
10 Giordanetto, F. & Kihlberg, J., J Med Chem 57, 278-295, doi:10.1021/jm400887j (2014).
11 Morrison, C. Nat. Rev. Drug Discov. 17, 531-533, doi:10.1038/nrd.2018.125 (2018).
12 Newman, D. J. & Cragg, G. M. J Nat Prod 83, 770-803, doi:10.1021/acs.jnatprod.9b01285 (2020).
13 LaBonte, J., et al., Nat. Rev. Drug Discov. 2, 345-346, doi:10.1038/nrd1091 (2003).
14 Petersen, J. et al. Nat Biotechnol 26, 335-341, doi:10.1038/nbt1389 (2008).
15 Kilby, J. M. et al. Nat. Med. 4, 1302-1307, doi:10.1038/3293 (1998).
16 Bird, G. H. et al. Proc. Natl. Acad. Sci. U.S.A 107, 14093-14098, doi:10.1073/pnas.1002713107 (2010).
17 Ng, S. & Derda, R. Organic and Biomolecular Chemistry 14, 5539-5545, doi:10.1039/c5ob02646f (2016).
18 Wang, X. S. et al. Angewandte Chemie International Edition 58, 15904-15909, doi:10.1002/anie.201908713 (2019).
19 Kale, S. S. et al. Nature Chemistry 10, 715-723, doi:10.1038/s41557-018-0042-7 (2018).
20 Guillen Schlippe, Y. V., et al., Journal of the American Chemical Society 134, 10469-10477, doi:10.1021/ja301017y (2012).
21 Hofmann, F. T., et al., J. Am. Chem. Soc. 134, 8038-8041, doi:10.1021/ja302082d (2012).
22 Hofmann, F. T., et al., Journal of the American Chemical Society 134, 8038-8041, doi:10.1021/ja302082d (2012).
23 Sakai, K. et al. et al., Nature Chemical Biology 15, 598-606, doi:10.1038/s41589-019-0285-7 (2019).
24 Fleming, S. R. et al. et al., J. Am. Chem. Soc., doi:10.1021/jacs.0c01576 (2020).
25 Scott, J. K. & Smith, G. P. et al., Science 249, 386-390, doi:10.1126/science.1696028 (1990).
26 Urban, J. H. et al. Nat. Commun. 8, 1500, doi:10.1038/s41467-017-01413-7 (2017).
27 Hetrick, K. J., et al., ACS Cent Sci 4, 458-467, doi:10.1021/acscentsci.7b00581 (2018).
28 Owens, A. E., et al., doi:10.1021/acscentsci.9b00927 (2019).
29 Tiede, C. et al. et al., Protein Engineering, Design and Selection 27, 145-155, doi:10.1093/protein/gzu007 (2014).
30 Barderas, R. & Benito-Peña, E. et al., Anal. Bioanal. Chem. 411, 2475-2479, doi:10.1007/s00216-019-01714-4 (2019).
31 Barbas, C. F., et al., Proceedings of the National Academy of Sciences of the United States of America 88, 7978-7982, doi:10.1073/pnas.88.18.7978 (1991).
32 Beerli, R. R. et al. Proceedings of the National Academy of Sciences of the United States of America 105, 14336-14341, doi:10.1073/pnas.0805942105 (2008).
33 Nixon, A. E., et al., MAbs 6, 73-85, doi:10.4161/mabs.27240 (2014).
34 Heinis, C., et al., Nat Chem Biol 5, 502-507, doi:10.1038/nchembio.184 (2009).
35 Millward, S. W., et al., ACS Chem Biol 2, 625-634, doi:10.1021/cb7001126 (2007).
36 Frankel, A., et al., Chem Biol 10, 1043-1050, doi:10.1016/j.chembiol.2003.11.004 (2003).
37 Shah, N. H. & Muir, T. W. Chem. Sci. 5, 446-461, doi:10.1039/C3SC52951G (2014).
38 Stevens, A. J. et al. J. Am. Chem. Soc. 138, 2162-2165, doi:10.1021/jacs.5b13528 (2016).
39 Stynen, B., et al., Microbiol. Mol. Biol. Rev. 76, 331-382, doi:10.1128/MMBR.05021-11 (2012).
40 Miranda, E. et al. J. Am. Chem. Soc. 135, 10418-10425, doi:10.1021/ja402993u (2013).
41 Yang, X. et al. Nat. Chem. Biol. 14, 375-380, doi:10.1038/s41589-018-0008-5 (2018).
42 Tavassoli, A. Curr. Opin. Chem. Biol. 38, 30-35, doi:10.1016/j.cbpa.2017.02.016 (2017).
43 Pu, J., Zinkus-Boltz, J. & Dickinson, B. C. Nature Chemical Biology 13, 432-438, doi:10.1038/nchembio.2299 (2017).
44 Horswill, A. R., et al., Proc. Natl. Acad. Sci. U.S.A 101, 15591-15596, doi:10.1073/pnas.0406999101 (2004).
45 Tominaga, M., et al., PLoS One 10, e0120243, doi:10.1371/journal.pone.0120243 (2015).
46 Kawai-Noma, S. et al. J. Biosci. Bioeng., doi:10.1016/j.jbiosc.2020.03.002 (2020).
47 Tavassoli, A. & Benkovic, S. J. Angew Chem Int Ed Engl 44, 2760-2763, doi:10.1002/anie.200500417 (2005).
48 Yao, Z. et al. Nature Communications 11, 2440-2440, doi:10.1038/s41467-020-16299-1 (2020).
49 Pinto, F., et al., Nature Communications 11, 1-15, doi:10.1038/s41467-020-15272-2 (2020).
50 Letko, M., et al., Nat Microbiol 5, 562-569, doi:10.1038/s41564-020-0688-y (2020).
51 Walls, A. C. et al. Cell, doi:10.1016/j.cell.2020.02.058 (2020).
52 Hoffmann, M. et al. Cell, doi:10.1016/j.cell.2020.02.052 (2020).
53 Tortorici, M. A. & Veesler, D. Adv Virus Res 105, 93-116, doi:10.1016/bs.aivir.2019.08.002 (2019).
54 Huo, J. et al. Nat. Struct. Mol. Biol., doi:10.1038/s41594-020-0469-6 (2020).
55 Jiang, S., et al., Trends Immunol. 41, 355-359, doi:10.1016/j.it.2020.03.007 (2020).
56 Wec, A. Z. et al. Science, doi:10.1126/science.abc7424 (2020).
57 Renn, A., et al., Trends Pharmacol Sci, doi:10.1016/j.tips.2020.07.004 (2020).
58 Schoof, M. et al. doi:10.1101/2020.08.08.238469 (2020).
59 Cao, L. et al. Science, doi:10.1126/science.abd9909 (2020).
60 Zhang, G. et al. bioRxiv, 2020.2003.2019.999318, doi:10.1101/2020.03.19.999318 (2020).
61 Kreutzer, A. G. et al. bioRxiv (2020).
62 Zhao, J. & Jiang, X. Chinese Chemical Letters 29, 1079-1087 (2018).
63 Hudson, G. A. et al. J. Am. Chem. Soc., doi:10.1021/jacs.9b01519 (2019).
64 Roh, H., et al., Chembiochem 20, 1051-1059, doi:10.1002/cbic.201800678 (2019).
65 Rhodius, V. A. et al. Mol. Syst. Biol. 9, 702, doi:10.1038/msb.2013.58 (2013).
66 Pinto, F., et al., Nat. Commun. 11, 1529, doi:10.1038/s41467-020-15272-2 (2020).
67 Moll, U. M. & Petrenko, O. Mol. Cancer Res. 1, 1001-1008 (2003).
68 Kussie, P. H. et al. Science 274, 948-953, doi:10.1126/science.274.5289.948 (1996).
69 Li, C. et al. J. Mol. Biol. 398, 200-213, doi:10.1016/j.jmb.2010.03.005 (2010).
70 Meyer, A. J., et al., Nat. Chem. Biol., doi:10.1038/s41589-018-0168-3 (2018).
71 Zhang, G., et al., bioRxiv, doi:10.1101/2020.03.19.999318 (2020).
72 Hegemann, J. D., et al., ACS Synth. Biol., doi:10.1021/acssynbio.9b00080 (2019).
73 Precord, T. W., et al., ACS Chem Biol 14, 1981-1989, doi:10.1021/acschembio.9b00457 (2019).
74 Sardar, D., et al., Chem Biol 22, 907-916, doi:10.1016/j.chembiol.2015.06.014 (2015).
75 Zong, C., et al., ACS Chem. Biol. 11, 61-68, doi:10.1021/acschembio.5b00745 (2016).
76 Noy-Porat, T. et al. Nat. Commun. 11, 4303, doi:10.1038/s41467-020-18159-4 (2020).
77 Chen, J. et al. World J. Gastroenterol. 11, 6159-6164, doi:10.3748/wjg.vl1.i39.6159 (2005).
78 Glassey, E., et al., ACS Synth. Biol. (2020).
79 Zhang, Q. et al. Proc. Natl. Acad. Sci. U.S.A 111, 12031-12036, doi:10.1073/pnas.1406418111 (2014).
80 Funk, M. A. & van der Donk, W. A. Acc. Chem. Res. 50, 1577-1586, doi:10.1021/acs.accounts.7b00175 (2017).
81 van der Velden, N. S. et al. Nat. Chem. Biol., doi:10.1038/nchembio.2393 (2017).
82 Morinaka, B. I. et al. Science 359, 779-782, doi:10.1126/science.aao0157 (2018).
83 Naylor, M. R., et al., Curr. Opin. Chem. Biol. 38, 141-147, doi:10.1016/j.cbpa.2017.04.012 (2017).
84 Fellner, R. C., et al., Mol Cell Pediatr 3, 16, doi:10.1186/s40348-016-0044-8 (2016).
85 Barth, P. et al. J. Cyst. Fibros. 19, 299-304, doi:10.1016/j.jcf.2019.08.020 (2020).
86 Xia, S. et al Cell Res 30, 343-355, doi:10.1038/s41422-020-0305-x (2020).
87 Gavrish, E. et al. Chem. Biol. 21, 509-518, doi:10.1016/j.chembiol.2014.01.014
(2014).
88 Delgado, M. A., et al., J Bacteriol 183, 4543-4550, doi:10.1128/JB.183.15.4543-4550.2001 (2001).
89 Rao, J., et al., Journal of the American Chemical Society 122, 2698-2710, doi:10.1021/ja9926481 (2000).
90 Zhu, Y., et al., J. Virol. 94, doi:10.1128/JVI.00635-20 (2020).
91 Xia, S. et al. bioRxiv, doi:10.1101/2020.03.09.983247 (2020).
92 Cao, Y. et al. Cell 182, 73-84.e16, doi:10.1016/j.cell.2020.05.025 (2020).
93 Rogers, T. F. et al. Science 369, 956-963, doi:10.1126/science.abc7520 (2020).
94 Si, L. et al. doi:10.1101/2020.04.13.039917.
95 Segall-Shapiro, T. H., et al., Nat. Biotechnol., doi:10.1038/nbt.4111 (2018).
96 Patiny, L. & Borel, A. J Chem Inf Model 53, 1223-1228, doi:10.1021/ci300563h (2013).
97 Yan, R. et al. Science, doi:10.1126/science.abb2762 (2020).

### Example 5: Optimization of Peptide Binders

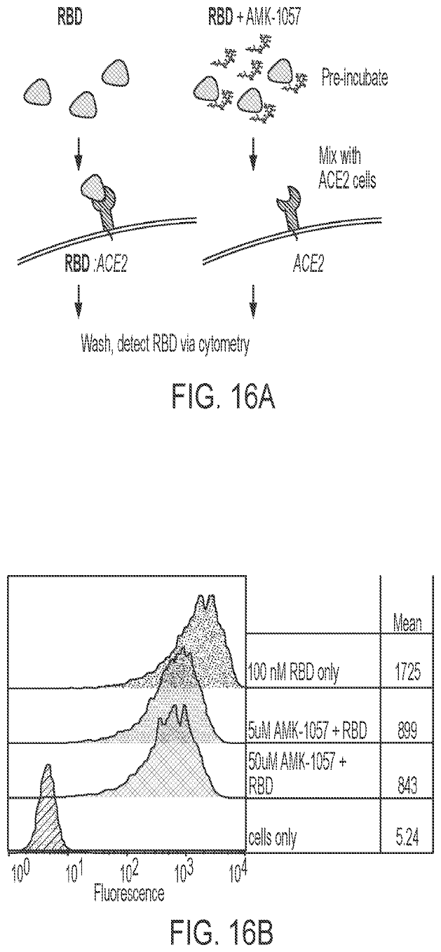

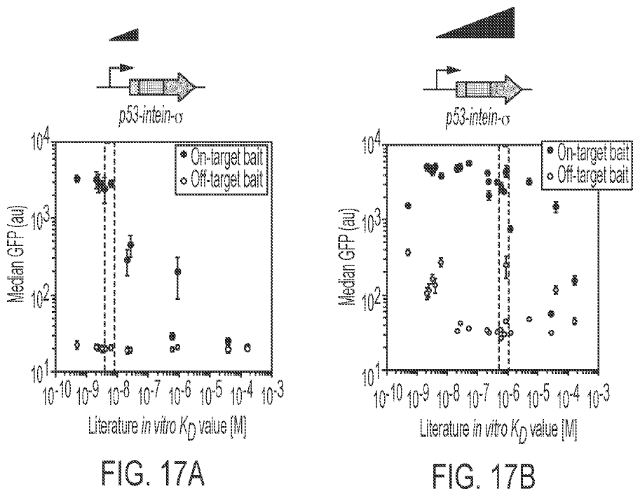

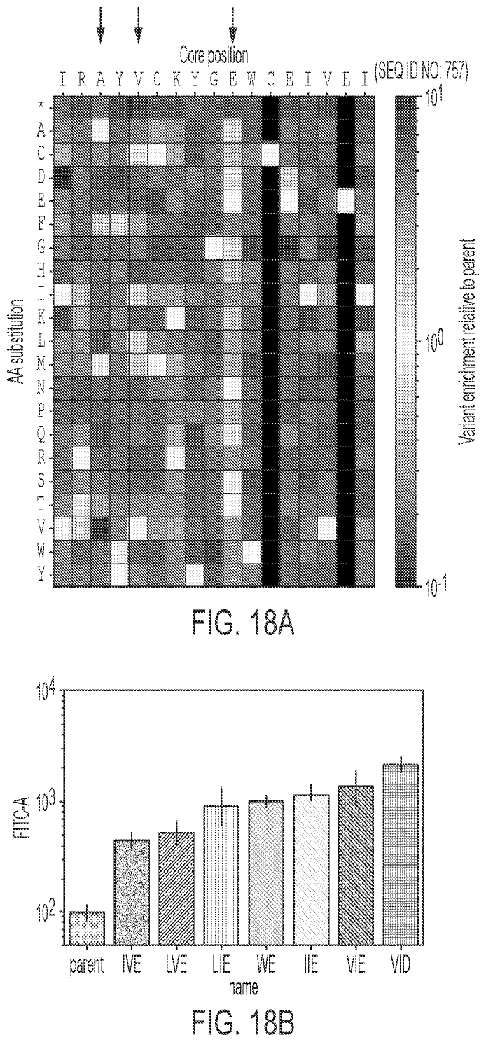

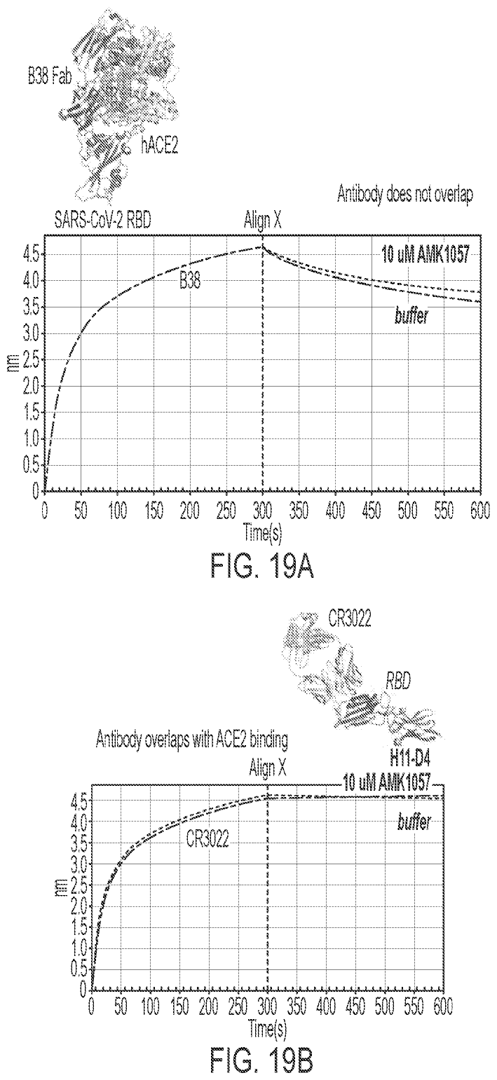

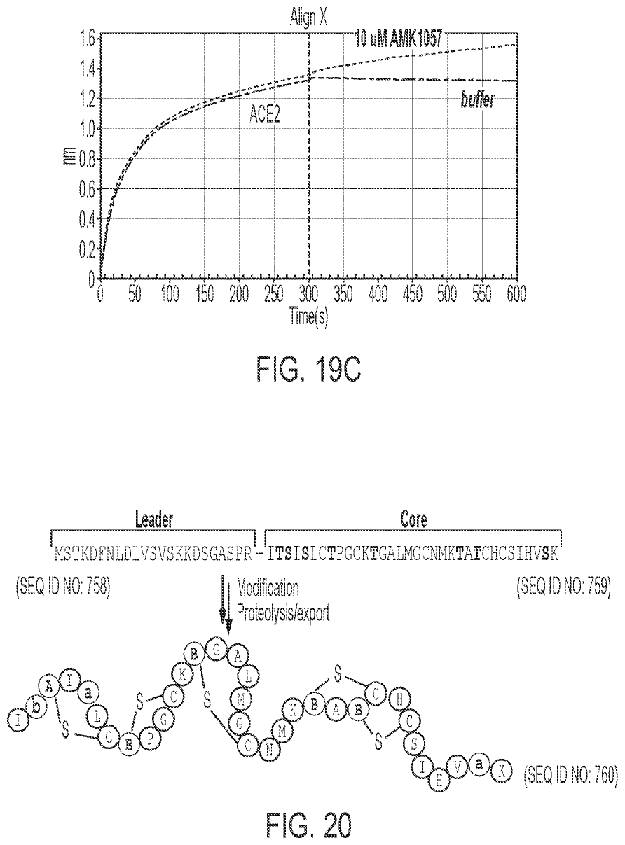

AMK-1057, a small peptide binder, was evaluated for cell competition between the Receptor Binding Domain (RBD) of the SARS-CoV-2 Spike protein and the human ACE2 receptor. RBD incubated with and without AMK-1057 was mixed with ACE2 cells, washed, and quantified via flow cytometry (FIG. 16A). Measurement of a fluorescence marker on the RBD demonstrated a slight decrease in binding of RBD to ACE2-expressing cells after the RBD was pre-incubated with the RBD, relative to RBD in the absence of AMK-1057, and the effect was stronger with higher concentration of AMK-1057 (FIG. 16B). The results demonstrate that the high nanomolar binding of AMK-1057 to RBD is not sufficient to robustly block RBD-ACE2 binding. A two-hybrid system was constructed to evaluate the effect of construct expression level on binding. Peptides with published K_{D} values were tested in the presence of on-target or off-target baits under conditions in which the peptides were expressed at a low level (FIG. 18B) or a high level (FIG. 18C). The results demonstrated that expression level of a given peptide can affect the ability to detect its binding to bait, and that lower expression allowed observation of differences in binding between on-target and off-target baits, whereas higher expression masked this effect to some extent. Scanning site saturation mutagenesis was performed on the core residues of AMK-1057 and variant enrichment was monitored using next-generation sequencing (FIG. 19A). Three positions that showed positive variant enrichment relative to the parent sequence were selected (arrows in FIG. 19A) and constructs expressing various combinations of amino acid substitutions at these positions were generated and evaluated for binding via flow cytometry. Each of the tested combinations of AMK-1057 variants showed improved binding relative to the parent peptide (FIG. 19B). The results demonstrate that screening of peptides with individual amino acid substitutions allows prediction of improved peptides with multiple substitutions.

Bio-layer interferometry was used to assay AMK-1057 competition for binding to RBD in the presence of B38 and CR3022 antibodies as well as purified ACE2 for the purpose of mapping what region of the RBD AMK-1057 may bind. RBD binding to AMK-1057 was not affected by the presence of B38 (FIG. 20A), CR3022 (FIG. 20B), or ACE2 (FIG. 20C).

### Example 6: Large Scale Genome Mining of the Human Microbiome for Targeted Antibiotic Discovery

The human microbiome harbors substantial biosynthetic potential for specialized metabolites with roles in host-microbe and microbe-microbe interactions. Analysis of genomic sequence data from the Human Microbiome Project shows an untapped source of post-translationally modified peptides, a class of molecule demonstrated to have important effects on human health and disease. Genome mining approaches, wherein DNA sequences are synthesized de novo and heterologously expressed in chassis organisms, can be leveraged to access the molecules encoded in human microbiome sequence data. However, robust methods for large-scale interrogation of sequence space through DNA synthesis and heterologous expression have yet to be developed. Here, 78 biosynthetic gene clusters were selected for post-translationally modified peptides from a diverse set of human microbiome strains from all niches of the human body. Production of peptides was shown in a format suitable for screening their biological activity and novel molecules with unique spectra of antimicrobial activity against members of the human microbiome and pathogenic bacteria of clinical significance were identified. This work demonstrates that large-scale genome mining of peptidic natural products and functional assaying for their biological activity is possible through a DNA sequence-to-molecule pipeline.

Revealing how the human microbiome affects health at a mechanistic level will continue to be critical in understanding disease and developing new therapies¹. Discovery and characterization of specialized metabolites (small molecules, peptides) is of particular interest due to their important role in biological systems and pharmaceutical potential as standalone agents or effectors in cell-based therapeutics². Traditional approaches to the isolation of specialized metabolites from the human microbiome have been hampered by access to putative producing organisms and difficulties in eliciting production. A number of bioinformatics tools are now available to parse ever-increasing DNA sequence data, annotate biosynthetic gene clusters, and assign basic molecular predictions³. These tools make possible a “sequence-to-molecule” approach, wherein mining DNA sequence databases, selecting gene clusters for DNA synthesis, and heterologous expression can yield specialized metabolites of value. However, the rate of molecular production is orders of magnitude behind in silico identification of the encoding DNA. Production of molecules is handicapped by difficulties with the large size of many gene clusters, appropriate heterologous production hosts, and standardized approaches for their purification as well as structural elucidation⁴.

Ribosomally synthesized and post-translationally modified peptides (RiPPs) are a class of specialized metabolite particularly abundant in human microbiome DNA sequence data⁵⁻⁷. RiPPs are defined by a conserved biosynthetic logic wherein a precursor peptide (comprised of a “leader” and “core” region) is ribosomally produced, the core subsequently altered by modifying enzymes that often recognize sequence motifs in the leader, then ultimately processed and exported (FIG. 20). For example, lanthipeptides are polycyclic peptides defined by the presence of thioether macrocycles formed via addition of a cysteine thiol to dehydrated serine and threonine residues (dehydroalanine and dehydrobutyrine, respectively). Dehydration of the core peptide and subsequent cyclization are catalyzed by a single, bifunctional enzyme or by two separate proteins, depending on the class of lanthipeptide⁵. Lasso peptides are formed via a complex of 2-3 proteins that recognize leader motifs, cleave the leader peptide, and use the resulting free amine to form an isopeptide bond with a downstream carboxyl side chain from an Asp/Glu residue⁵. The resulting constrained peptides are not only structurally diverse but also enriched in biological activity. RiPPs produced by the human microbiome are responsible for a remarkable range of microbe-host interactions^{8,9} as well as microbe-microbe interactions¹⁰⁻¹³ with pronounced effects upon human health.

As of 2015, 100 lanthipeptides had been discovered from microbes¹⁴ and half that number of lasso peptides¹⁵. New computational approaches to RiPP genome mining have yielded impressive advances in the discovery of RiPP subclasses and scaffolds but actual molecular discovery is relatively low (˜1-5 molecules per report) and functional assaying is either absent or narrow in scope¹⁶⁻²¹. The flexible biosynthesis afforded by RiPPs has also led to a number of innovative strategies for generating large libraries around a given peptide scaffold linked to a functional output. These include libraries based on the lasso peptide microcin J25²², the thiopeptide thiocillin²³, and the lanthipeptides nisin, prochlorosin, haloduracin, and lacticin 481²⁴⁻²⁸. While of outstanding value, these approaches all require specialized assays and selections and do not exploit specific biological activities afforded by natural evolution. There is a need for higher throughput approaches to purify, express, and structurally annotate RiPPs that can then be tested in diverse functional assays. Here, an E. coli-based expression system was used to mine 78 RiPP gene clusters to generate 23 new lanthipeptides and lasso peptides from the human microbiome. The established pipeline was able to go from DNA sequence information to a structurally and functionally annotated molecule in relatively high-throughput. These 23 structurally annotated RiPPs, combined with 7 RiPPs with unknown modification, were demonstrated to have unique scaffolds and spectra of antimicrobial activity when tested against a large panel of human microbiome-associated strains. A subset of these RiPPs were shown to possess activity against multidrug resistant (MDR) clinical isolates of human pathogens, including vancomycin resistant Enterococcus and methicillin-resistant Staphylococcus aureus. This provides a robust method for accessing a vast and underexplored chemical space of the human microbiome.

### Results

### Selection of Human Microbiome RiPP Gene Clusters for Heterologous Expression

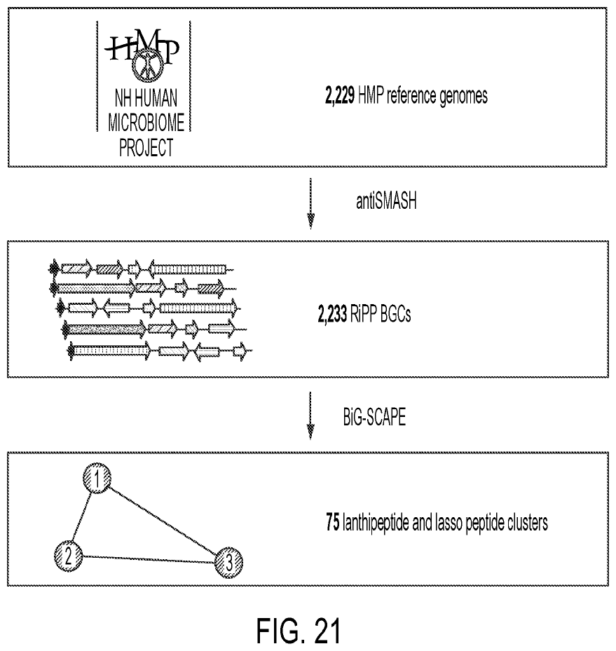

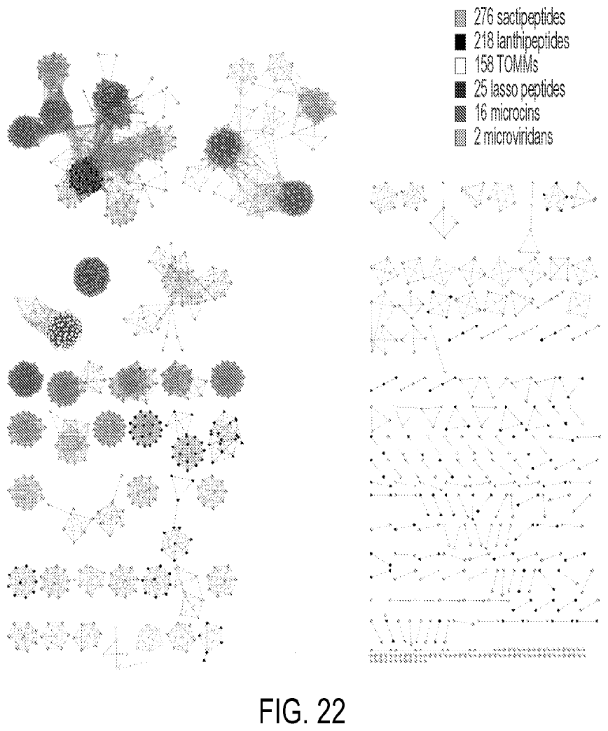

AntiSMASH²⁹ was used to identify 2,233 RiPP gene clusters from 2,231 genomes of the Human Microbiome Project (HMP)³⁰. BiG-SCAPE³¹ was then used to generate a sequence similarity map of these gene clusters to visualize the abundance of different subclasses of RiPP (FIG. 21 and FIG. 22). Previously identified RiPP gene clusters from the Joint Genome Institute (JGI)^{6,32} were also included in the analysis. Clusters were not prioritized based on any perceived contribution to health of a producing organisms; pathogens are an equally useful source of biologically active molecules with therapeutic potential^{33,34}. From this survey, it was decided to pursue genome mining of two RiPP subclasses enriched in the human microbiome: lanthipeptides and lasso peptides.

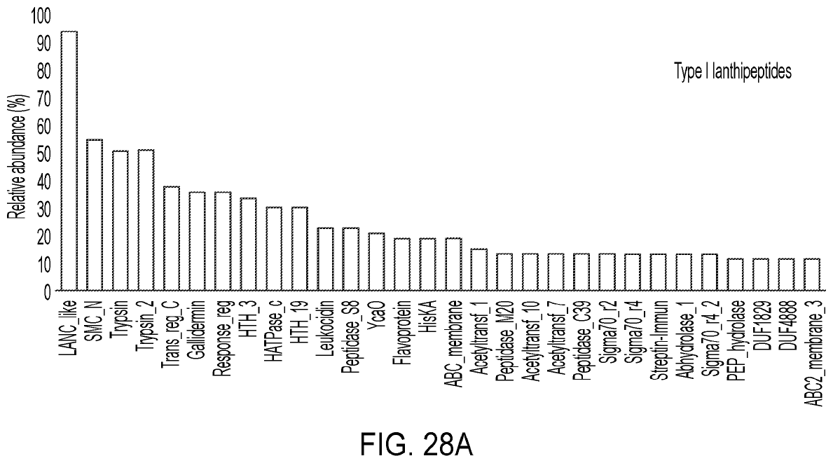

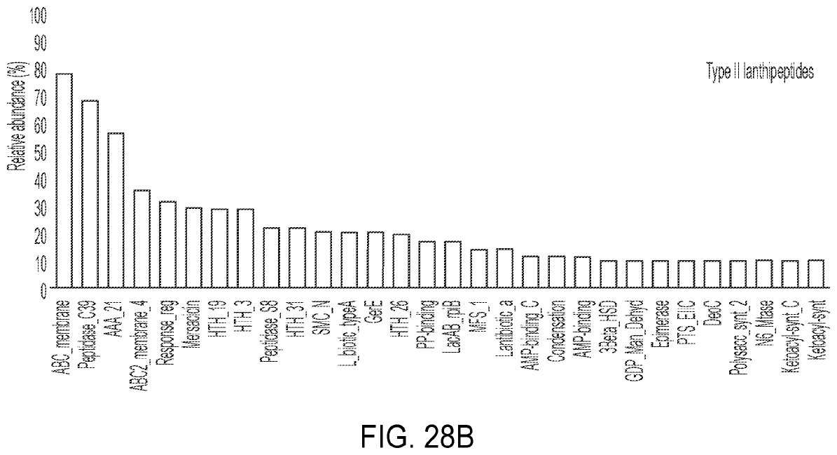

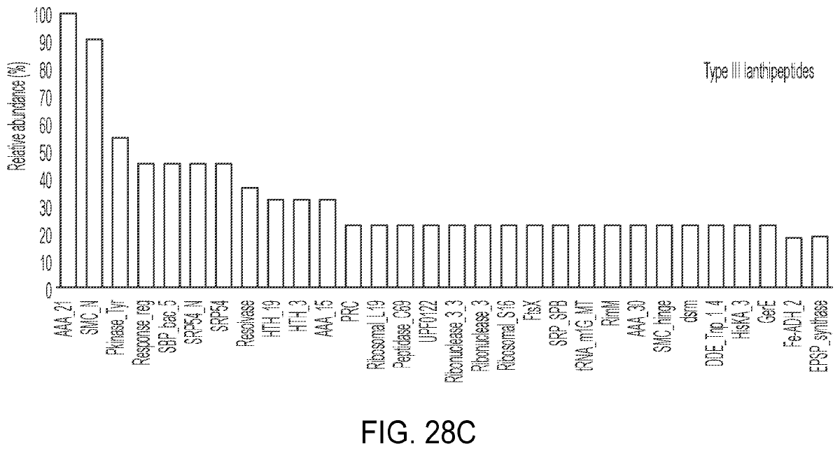

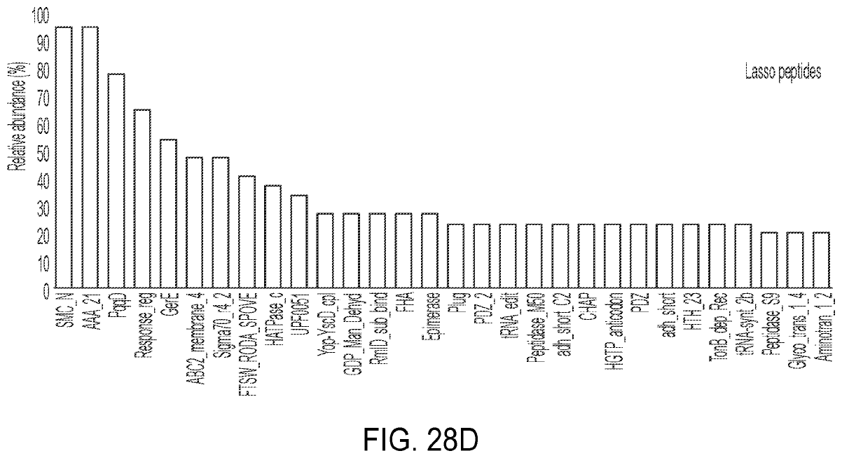

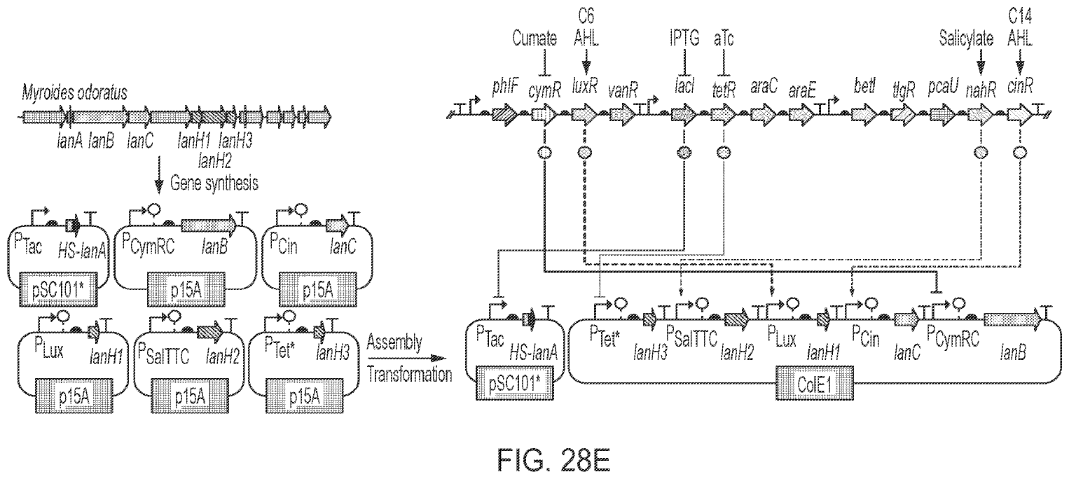

In addition to the defining biosynthetic enzymes described above (LanBC, LanM, LanK for lanthipeptides; LasBC for lasso peptides), “tailoring enzymes” that further chemically diversify peptides can be encoded in gene clusters. Tailoring enzymes can modify bioactivity of peptides and have promise in functioning as catalysts for engineering RiPPs³⁵ so open reading frames encoding putative tailoring enzymes were included in the mining workflow. Novel tailoring enzymes were not identifiable by existing in silico methods so a script was developed to identify and count the presence of all protein family (pfam) domains found in gene clusters annotated by AntiSMASH. These pfam counts were converted to relative abundance by dividing raw counts by the presence of core biosynthetic enzymes (lanBC/M/K; lasBC) and rank-ordered to profile prevalence of certain pfam domains in each subclass of RiPP investigated here (FIGS. 28A-28D). Certain pfam domains feasibly associated with biosynthesis (acetyltransferases, flavoproteins, epimerases, methyltransferases, dehydrogenases, aminotransferases, and glycosyltransferases) were modestly enriched. This analysis was coupled to manual inspection of each cluster for putative tailoring enzymes, then candidate genes were synthesized and assembled into single expression plasmids using an orthogonal set of inducible promoters³⁶ (FIG. 28E).

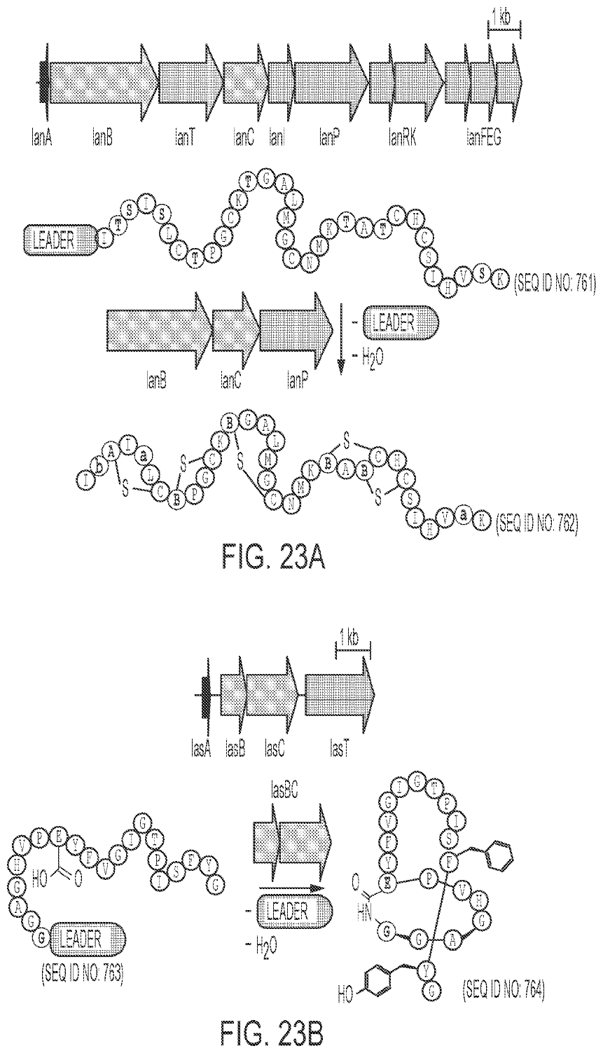

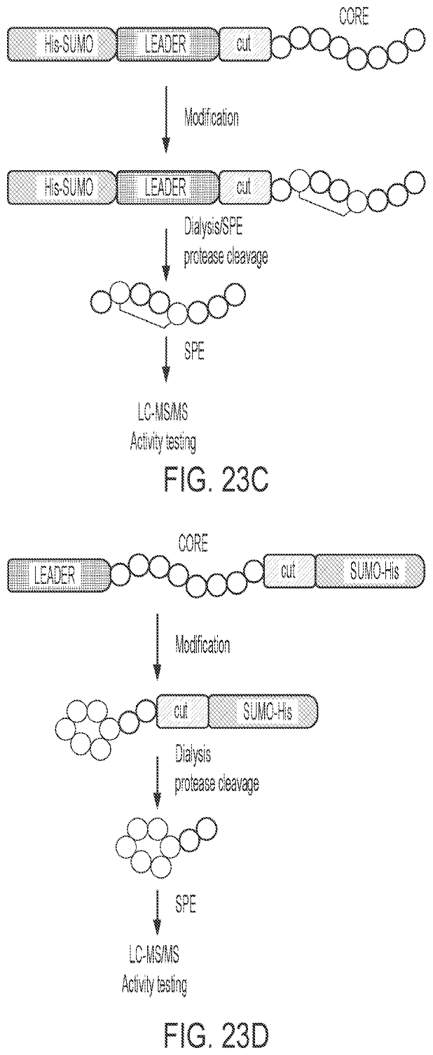

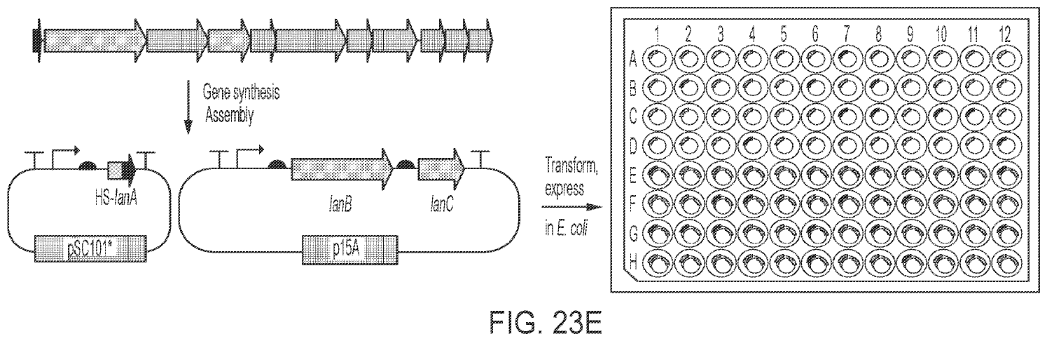

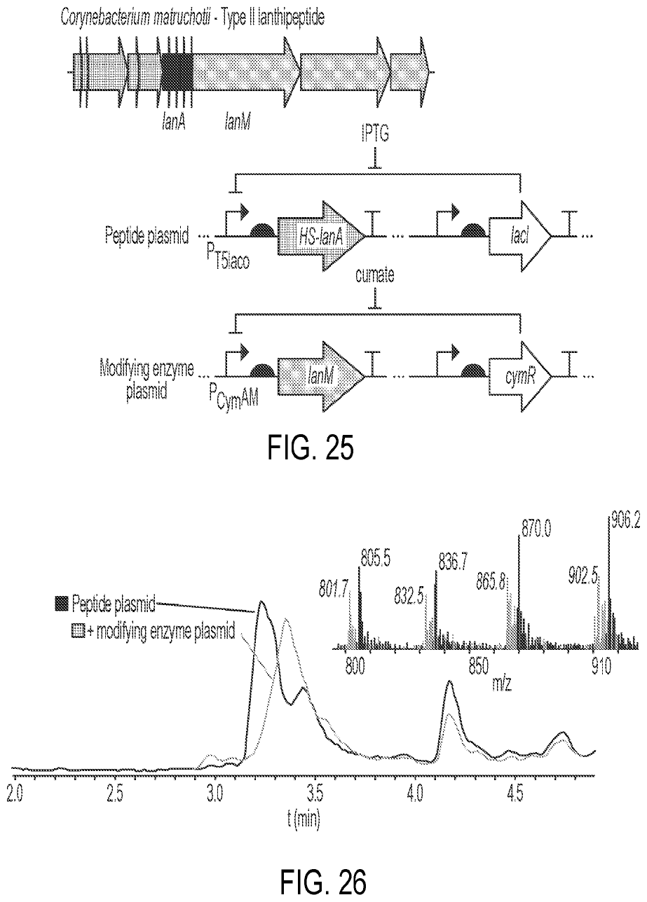

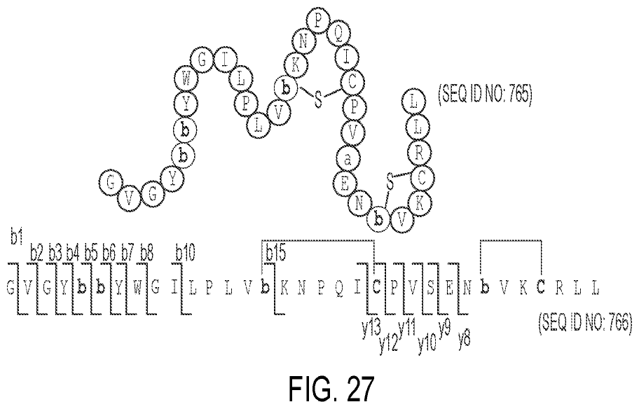

78 gene clusters were selected from 68 diverse organisms spanning 6 classes and occupying airway, gastrointestinal (GI) tract, oral, skin, and urogenital (UG) tract microbiomes (FIG. 24, Table 11). A two-plasmid expression system was used wherein putative precursor peptides and modifying enzymes are synthesized and assembled in plasmids under control of inducible promoters (FIG. 25), singly- or doubly-transformed into E. coli, and analyzed by liquid chromatography-mass spectrometry (LC-MS) for retention and mass shifts indicative of peptide modification (FIG. 26)³⁷. Peptides were engineered to possess either an N-terminal or C-terminal (lanthipeptides and lasso peptides, respectively) hexa-histidine-small ubiquitin-like modifier (SUMO) tag for affinity purification and increased peptide stability (HS-tag) (FIGS. 23A and 23B). Peptides were also engineered to possess protease sites in order to remove the HS-tag and leader peptide (FIGS. 23C and 23D), which enabled structural annotation of lanthipeptides through hypothetical structure enumeration and evaluation (HSEE)³⁸ (FIG. 27). The entire process of assembly, transformation, growth, and purification was optimized for the use of 96-well microtiter plates (FIG. 23E)³⁷.

TABLE 11
Bacterial strains used in Example 6.

| Species | Niche | Strain | Source | Media | Growth |
|---|---|---|---|---|---|
| Streptococcus pneumoniae | Airways | Streptococcus pneumoniae | Ribbick lab | TSBb | anaerobic |
| | | TIGR4 | | | |
| Dolosigranulum pigrum | Airways | Dolosigranulum | ATCC | TSBb | aerobic |
| | | pigrum Aguirre et al. | | | |
| | | (ATCC ® 51524 ™) | | | |
| Staphylococcus caprae | Airways | Staphylococcus | ATCC | TSBb | aerobic |
| | | caprae (ATCC ® 55133 ™) | | | |
| Staphylococcus capitis | Airways, | Staphylococcus capitis | Voigt lab | TSBb | aerobic |
| | skin | TA281 (JAB794) | | | |
| Staphylococcus epidermis | Airways, | Staphylococcus epidermidis | Voigt lab | TSBb | aerobic |
| | skin | TA278 (JAB793) | | | |
| Streptococcus infantarius | Gut | Streptococcus infantarius | Voigt lab | TSBb | anaerobic |
| | | subsp. infantarius ATCC- | | | |
| | | BAA-102 (JAB516) | | | |
| Bacteroides_{—}dorei | Gut | aa_0143_0002_h6 | OpenBiome | BHIs | anaerobic |
| Bacteroides_{—}faecis | Gut | aa_0143_0089_f9 | OpenBiome | BHIs | anaerobic |
| Bacteroides_{—}thetaiotaomicron | Gut | af_0058_0071_a4 | OpenBiome | BHIs | anaerobic |
| Bifidobacterium_{—}adolescentis | Gut | ao_0067_0069_a1 | OpenBiome | BHIs | anaerobic |
| Bifidobacterium_{—}longum | Gut | am_0171_0090_c1 | OpenBiome | BHIs | anaerobic |
| Citrobacter_{—}amalonaticus | Gut | ao_0067_0062_a8 | OpenBiome | BHIs | anaerobic |
| Enterococcus_{—}avium | Gut | ao_0067_0069_c1 | OpenBiome | BHIs | anaerobic |
| Enterococcus_{—}durans | Gut | am_0171_0068_e1 | OpenBiome | BHIs | anaerobic |
| Enterococcus_{—}mundtii | Gut | am_0171_0068_d4 | OpenBiome | BHIs | anaerobic |
| Leuconostoc_{—}lactis | Gut | aa_0143_0055_c12 | OpenBiome | BHIs | anaerobic |
| Paeniclostridium_{—}sordellii | Gut | av_0103_0069_f8 | OpenBiome | BHIs | anaerobic |
| Parabacteroides_{—}distasonis | Gut | cx_0004_0077_a10 | OpenBiome | BHIs | anaerobic |
| Parabacteroides_{—}goldsteinii | Gut | aa_0143_0055_a8 | OpenBiome | BHIs | anaerobic |
| Pediococcus_{—}acidilactici | Gut | cx_0004_0082_e12 | OpenBiome | BHIs | anaerobic |
| Ruthenibacterium_{—}lacta- | Gut | am_0070_0084_c5 | OpenBiome | BHIs | anaerobic |
| tiformans | | | | | |
| Sellimonas_{—}intestinalis | Gut | am_0224_0084_c8 | OpenBiome | BHIs | anaerobic |
| Veillonella_{—}dispar | Gut | bj_0095_0068_g5 | OpenBiome | BHIs | anaerobic |
| Streptococcus sobrinius | oral | Streptococcus sobrinius 6715 | Ribbick lab | TSBb | anaerobic |
| Streptococcus mitis | oral | Streptococcus | ATCC | TSBb | anaerobic |
| | | mitis Andrewes and Horder | | | |
| | | emend. Judicial Commission | | | |
| | | (ATCC ® 49456 ™) | | | |
| Streptococcus gordonii | oral | Streptococcus gordonii Kilian | ATCC | TSBb | anaerobic |
| | | et al. (ATCC ® 33399 ™) | | | |
| Streptococcus mutans | oral | Streptococcus mutans UA159 | Ribbick lab | TSBb | anaerobic |
| Rothia dentocariosa | oral | Rothia dentocariosa (Onishi) | ATCC | TSBb | aerobic |
| | | Georg and Brown | | | |
| | | (ATCC ® 17931 ™) | | | |
| Corynebacterium striatum | Skin | Corynebacterium | ATCC | TSBb | aerobic |
| | | striatum (Chester) Eberson | | | |
| | | (ATCC ® 6940) | | | |
| Micrococcus luteus | Skin | Micrococcus | Wright lab | TSBb | aerobic |
| | | luteus (Schroeter) Cohn | | | |
| | | (ATCC ® 10240 ™) | | | |
| Staphylococcus aureus | Skin | Staphylococcus aureus subsp. | Voigt lab | TSBb | aerobic |
| | | aureus ATCC-19685 | | | |
| | | (JAB849) | | | |
| Staphylococcus hominis | Skin | Staphylococcus | ATCC | TSBb | aerobic |
| | | hominis subsp. hominis Kloos | | | |
| | | and Schleifer | | | |
| | | (ATCC ® 27844 ™) | | | |
| Streptococcus dysgalactiae | Skin | Streptococcus dysgalactiae | Voigt lab | TSBb | aerobic |
| | | TA380 (JAB792) | | | |
| Streptococcus sanguinis | Skin, oral | Streptococcus | Ribbick lab | TSBb | anaerobic |
| | | sanguinis White and Niven | | | |
| | | emend. Kilian et al. | | | |
| | | (ATCC ® 10556 ™) | | | |
| Lactobacillus crispatus JV-V01 | vagina | L. crispatus JV-V01 | Mitchell lab | MRS | anaerobic |
| Lactobacillus jensenii ATCC | vagina | L. jensenii ATCC 25258 | Mitchell lab | MRS | anaerobic |
| 25258 | | | | | |
| Lactobacillus gasseri ATCC | vagina | L. gasseri ATCC 33323 | Mitchell lab | MRS | anaerobic |
| 33323 | | | | | |
| Acinetobacter baumannii | pathogen | 0033 | CDC | TSBb | aerobic |
| Aspergillus fumigatus | pathogen | 0731 | CDC | SDA | aerobic |
| Campylobacter jejuni | pathogen | 0412 | CDC | TSBb | aerobic |
| Candida albicans | pathogen | 0761 | CDC | SDA | aerobic |
| Enterococcus faecalis | pathogen | 0679 | CDC | TSBb | aerobic |
| Enterococcus faecium | pathogen | 0572 | CDC | TSBb | aerobic |
| Escherichia coli | pathogen | 0011 | CDC | TSBb | aerobic |
| Klebsiella pneumoniae | pathogen | 0112 | CDC | TSBb | aerobic |
| Pseudomonas aeruginosa | pathogen | 0508 | CDC | TSBb | aerobic |
| Salmonella Typhimurium | pathogen | 0408 | CDC | TSBb | aerobic |
| Staphylococcus aureus | pathogen | 0215 | CDC | TSBb | aerobic |

E. coli is an Effective Chassis Organism for Genome Mining of RiPPs

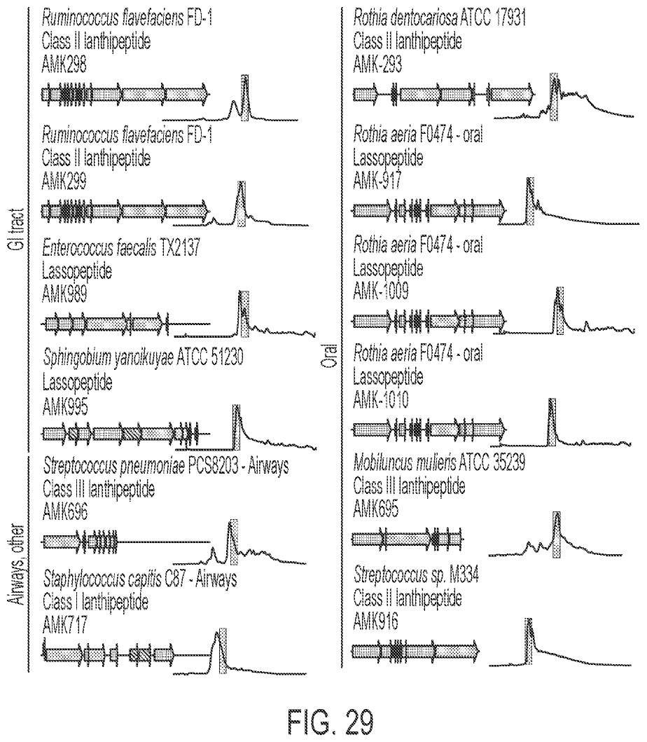

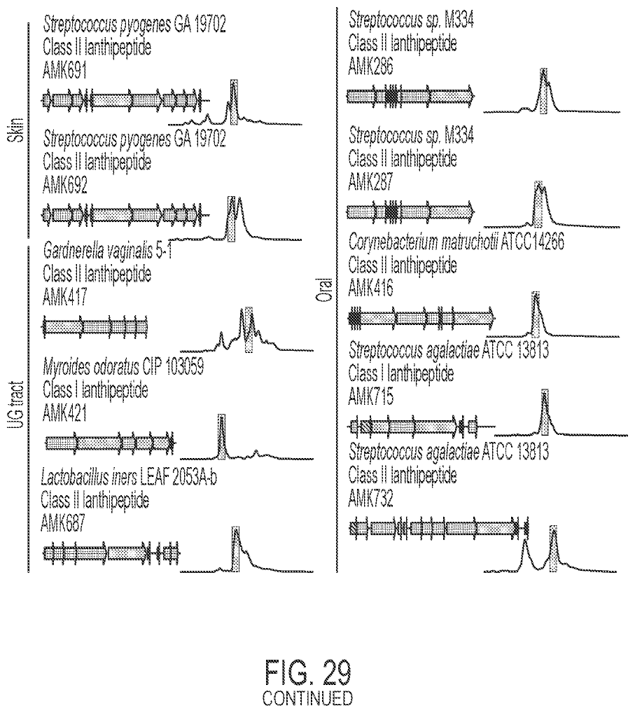

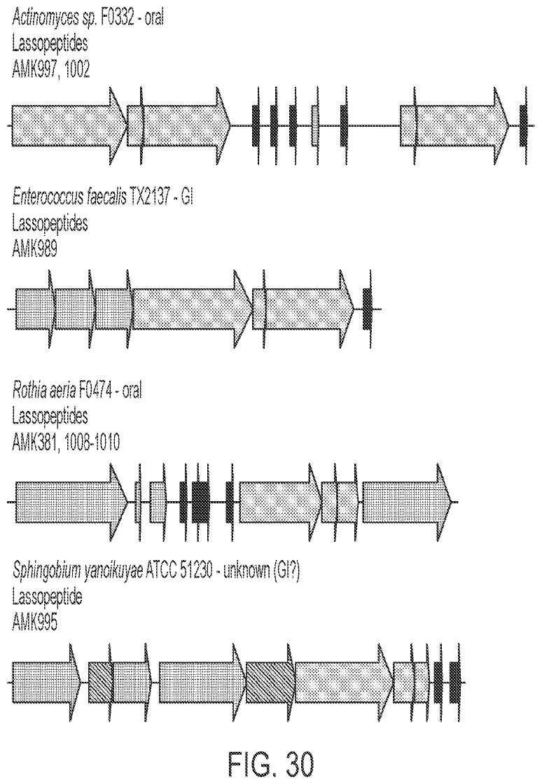

Application of this workflow to the selected gene clusters resulted in the detection and subsequent structural annotation of 18 lanthipeptides and 5 lasso peptides (FIG. 29). FIG. 29 shows total ion chromatograms (TICs) of TALON purified microtiter plate expressions. The HS-peptides were clearly identifiable via their mass shifts (water losses, −18 Da) using a low-resolution instrument and the detected peaks are highlighted. All of the peptides highlighted in FIG. 29 were successfully expressed, purified, dialyzed, cleaved, and SPE purified at 0.5 L scale and then subject to liquid chromatography tandem mass spectrometry (LC-MS/MS). Structures were annotated using HSEE, which is a method wherein all hypothetical modified peptide structures are enumerated in silico and compared to observed fragments from the MS/MS experiment^{37,38}. N-Ethylmaleimide was used to determine the extent of cyclization for lanthipeptides and infer macrocycle topology via absence of fragmentation, as was demonstrated previously³⁹⁻⁴⁵. RiPPs from all microbiome niches were discovered but with varying rates of success: airways/other (2/11, 18%), GI (4/29, 14%), oral (11/23, 48%), skin (2/7, 29%), and UG (3/6, 50%).

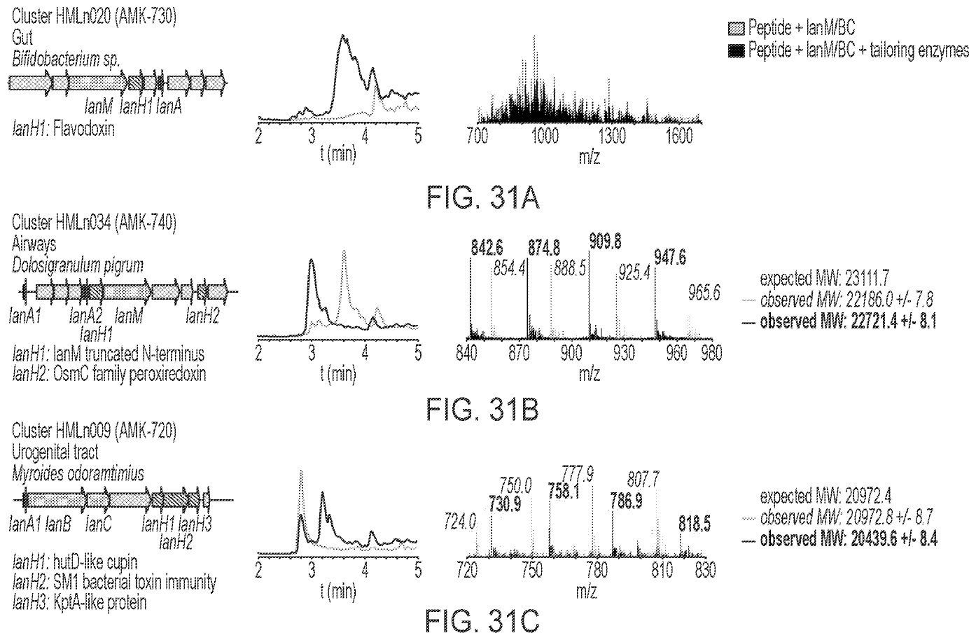

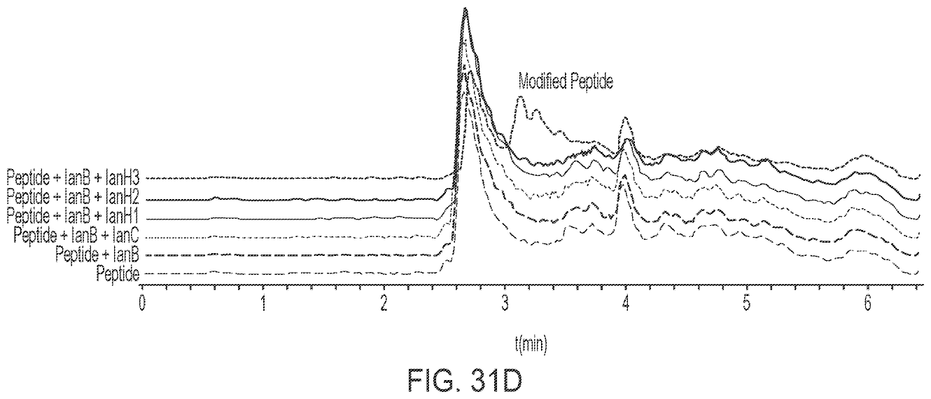

7 lanthipeptide clusters generated retention/mass shifts in the presence of modifying enzymes but mass shifts weren't consistent with known modification patterns. Of particular interest were several producing strains that showed modifications via retention time/mass shift when putative tailoring enzymes were expressed (FIG. 31A-31D). For both above examples the mass spectra could be difficult to deconvolute, but expression differences were clear with the addition of modifying enzyme(s), as in HMLn020 (sAMK-730 from Bifidobacterium sp.) (FIG. 31A). In another example, expression of the core biosynthetic enzymes and putative peptide lanA2 from cluster HMLn034 (sAMK-740 from Dolosigranulum pigrum) resulted in production and mass detection of a 6×-dehyrated peptide lacking the last eight residues of its C-terminus, presumably from unanticipated proteolysis via E. coli enzymes. Additional expression of putative tailoring enzymes resulted in the production and detection of a higher molecular weight peptide consistent with a modified sequence lacking the last three residues of its C-terminus (FIG. 31B). Most strikingly, expression of the core biosynthetic enzymes and a putative peptide from cluster HMLn009 (sAMK-720 Myroides odoratimimus) did not result in any modification of the precursor peptide but additional expression of three putative tailoring enzymes resulted in a mass shift of −533.2 Da (FIG. 31C). Expression of one gene in particular, a KptA-like protein, was shown to be necessary for the observed modification. Homologs of KptA have been implicated in peptide modification⁴⁶. Selective induction of the Marionette orthogonal expression system enabled the simultaneous expression of all putative genes and systematic interrogation of their contribution to biosynthesis, demonstrating its utility in genome mining via construction of a single strain.

A diverse selection of producing organisms were selected from which to mine lanthipeptide sequences for heterologous expression and whether gene clusters from particular genera were more or less suitable for expression in E. coli was investigated. To this end, a taxonomic tree of all lanthipeptide-producing organisms (with E. coli BL21 for reference) selected for this study was generated. Strains from which that successfully produced a RiPP were highlighted to detect trends (FIG. 32A). Lanthipeptides originating from all Classes of strains used in this study were successfully expressed and no obvious trends in failures or successes observed. The biosynthesis of type I lanthipeptides was next considered. Type I lanthipeptides are unusual in their requirement for glutamyl-tRNA (tRNAGlu) to activate Ser/Thr residues for dehydration (FIG. 32B) as opposed to using ATP, as in type II-IV lanthipeptide biosynthesis (FIG. 32C)⁴⁷. Sequence differences in tRNAGlu have been shown to be important in heterologous expression of lanB-type enzymes⁴⁸. Sequences for tRNAGlu from all species mined for type I lanthipeptide biosynthesis were used to generate a phylogenetic tree. Sequence homology of tRNAGlu was not important in analysis of successful production using E. coli as chassis, but no gene clusters from strains possessing alternative anticodon loops (CTC as opposed to TTC) were successfully produced, consistent with previous reports and predictions⁴⁸.

### Heterologous Expression of RiPP Gene Clusters Suitable for Functional Assaying

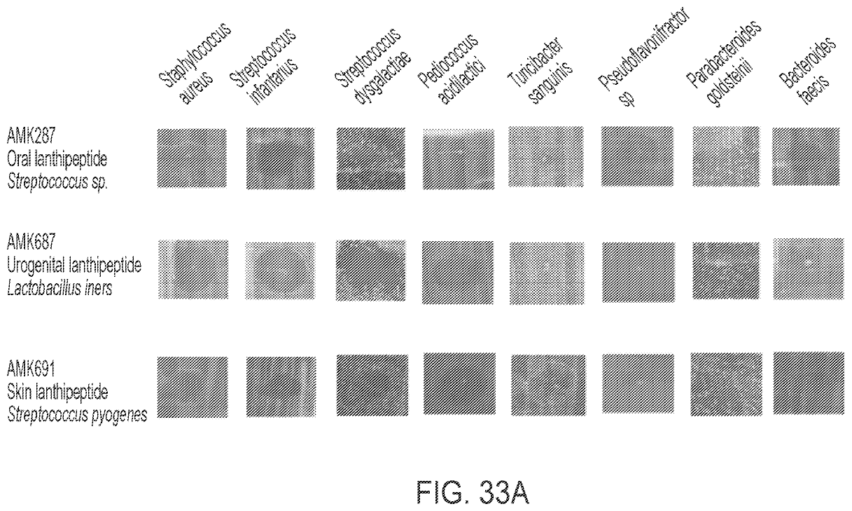

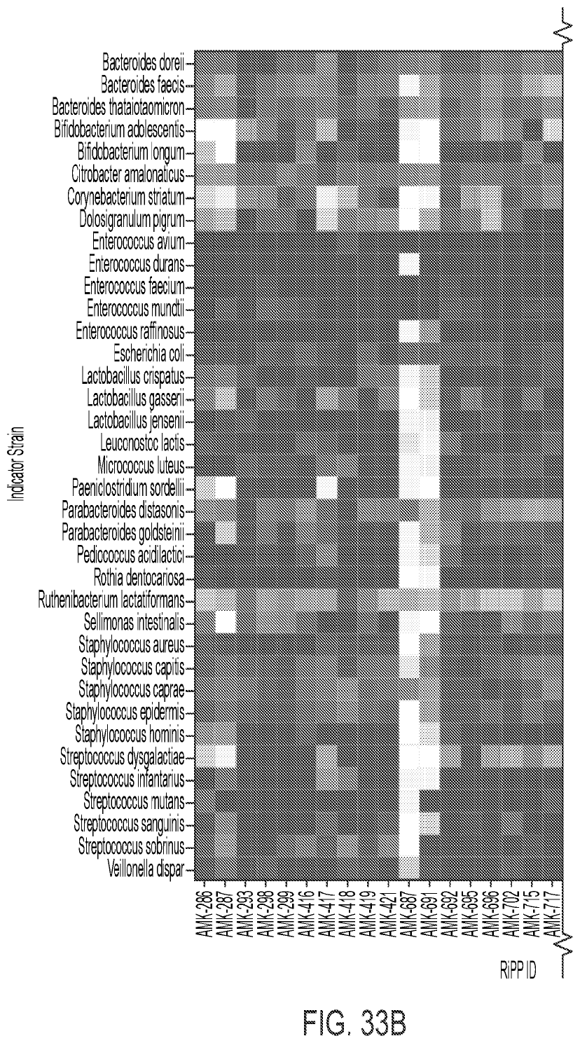

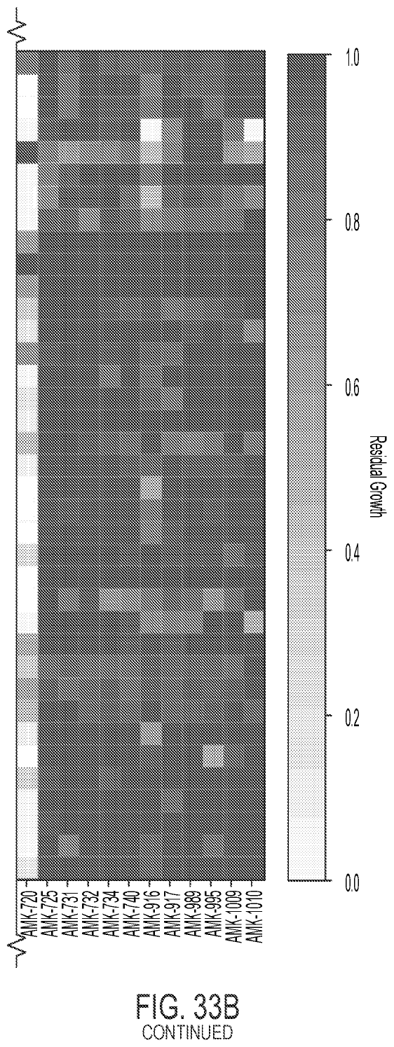

96-well microtiter growths (2×1 mL TB media) were purified and processed and optimal conditions for assaying biological activity were considered. Agar plate-based assays that demonstrate antimicrobial activity via zones of inhibition are an ideal method since compounds do not suffer dilution as in liquid-based readouts of optical density. Microtiter-purified RiPPs were initially tested against a subset of human microbiome-associated strains (Staphylococcus aureus, Streptococcus infantarius, Streptococcus dysgalactiae, Pediococcus acidilactici, Pseudofalnovifractor spp., and Bacteroides faecis) to assess this plate-based method and several producing strains (sAMK-287, sAMK-687, sAMK-691) showed varying zones of inhibition against this initial test set of indicator strains (FIG. 33A). While clear activity was observed in some cases, inconsistencies in colony density ruled out this method as a systematic means for detecting antimicrobial activity of RiPPs against a diverse panel of strains.

To streamline functional assaying, 96-well microtiter growths were optimized for a large collection of indicator strains sourced from a variety of niches found in the human microbiome (Table 11). The large-scale antimicrobial profiling of 30 SPE purified RiPPs (including both peptides that were confirmed via the structural annotation pipeline as well as putative modified peptides) showed that 8/30 demonstrated unique antimicrobial “fingerprints”. Of these active peptides, 7/8 could be grouped either through a common source cluster (AMK-286, 287, 916; AMK-917, 1009, 1010) or a common structural scaffold (AMK-417, 687, 691). The eighth, AMK-720, is an uncharacterized modified peptide that showed exceptionally broad antimicrobial activity. The structure and biosynthesis of AMK-720 are still under investigation, but structure-function relationships for the other three groups of peptides are described below.

### Human Microbiome RiPPs Possess Unique Antimicrobial Fingerprints

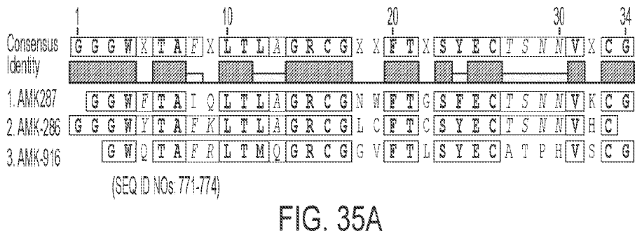

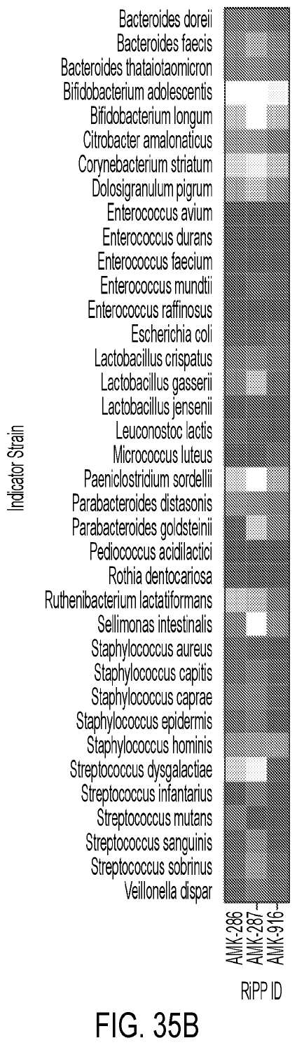

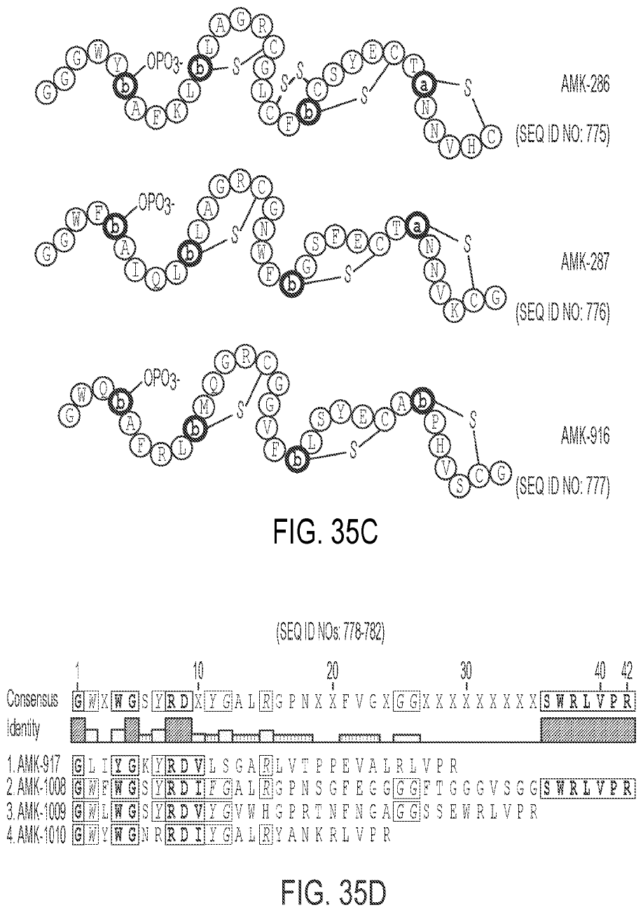

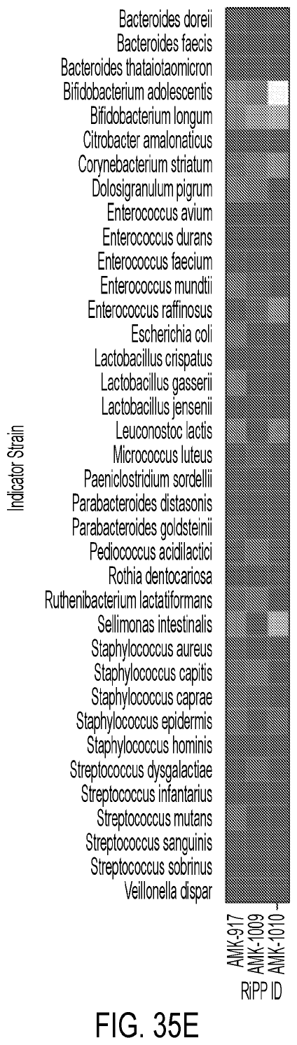

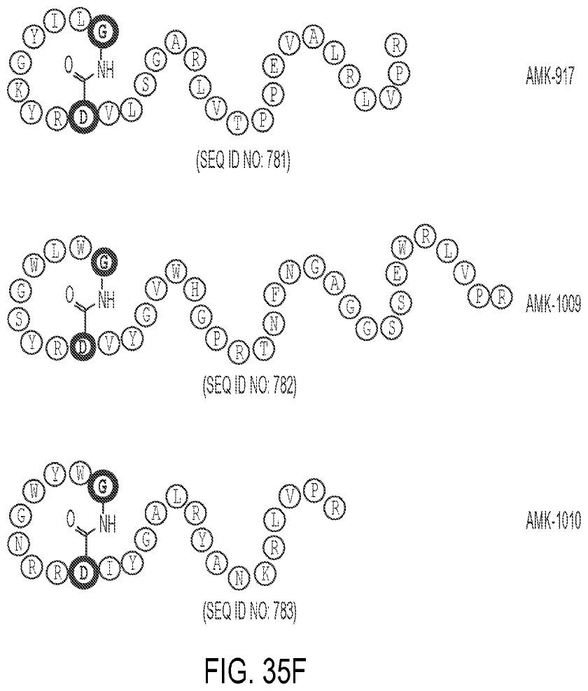

The type II lanthipeptides AMK-286, AMK-287, and AMK-916 were based on genes from an oral strain of Streptococcus and share identical modification profiles (FIGS. 35A and 35C), including the consistent presence of a phosphoryl group on the most N-terminal threonine residue, which is a novel observation for enzymes of this class. Several features were apparent from the antimicrobial activity patterns of these related molecules (FIG. 35B). They (in particular AMK-287) demonstrated pronounced activity against strains of the alimentary tract, including Bifidobacterium adolescenits, Bifidobacterium longum, Sellimonas intestinalis, and Streptococcus dysgalactiae. The interplay between the oral microbiome and alimentary tract is complex and elucidating chemical mechanisms by which oral strains can disrupt and colonize the gut is critical to understanding the roles of certain strains in health and disease⁴⁹. B. adolescenits and B. longum, for instance, are associated with anxiety and depression in mammals via substantial production of gamma-Aminobutyric acid⁵⁰. These Streptococcus-derived RiPPs also demonstrated remarkably narrow spectrum activity with respect to other Streptococci (only significant activity observed against 1/5 Streptococcal strains). This suggested that an oral-derived Streptococcus produced a suite of molecules lacking activity against closely related members of the oral microbiomes⁵¹.

The lasso peptides AMK-917, 1009, and 1010 are from an oral strain of Rothia dentocariosa and exhibit conserved primary amino acid sequence about the lariat structure, with some degeneracy (FIGS. 35D-35F). The predicted amino acid sequences of these lasso peptides are substantially longer than others that were expressed in this study (Table 12) and antimicrobial activity tracked inversely with length of the core (FIG. 35E). The use of a C-terminal Factor Xa cleavage site (which leaves an “RLVPR (SEQ ID NO: 714)” scar) likely further exacerbated this negative trend. AMK-1008 was another predicted lasso peptide from the same gene cluster that was not observed during heterologous expression and had a much longer core sequence (AMK-917, 24 aa; AMK-1008, 37 aa; AMK-1009, 30 aa; and AMK-1010, 19 aa). Based on the amino acid sequence alignment (FIG. 35D), it was determined that AMK-1008 and AMK-1009 may be processed by non-cluster associated proteases that cleave before or after the “GG” at position 25. As such, discovery and application of scarless C-terminal proteases as well as iterations on core sequences used in heterologous expression will likely be important in genome mining lasso peptides.

Amino acid sequence alignments showed that AMK-417, 687, and 691 belong to the same family of RiPPs as lacticin 481 and the structural annotation was consistent with a similar cyclization pattern (FIGS. 57A-57C). Because AMK-687 displayed such remarkable antimicrobial activity and 2/5 strains were from the vaginal microbiome, the activity of these peptides was tested against Lactobacillus crispatus, a dominant member of the healthy vaginal microbiome⁵². AMK-687 displayed even more pronounced activity against this related strain while AMK-691 also demonstrated activity but other lanthipeptides tested were inactive (FIG. 57D). The strong activity of AMK-687 was noteworthy because Lactobacillus iners is implicated in transition of a healthy vaginal microbiome to an unhealthy one via depletion of the predominant Lactobacillus crispatus and related strains, but the exact mechanisms by which this dysbiosis occurs are largely unknown⁵³.

TABLE 12
Microbiome RiPPs

| Strain | | | SEQ ID |
|---|---|---|---|
| designation | Producing organism | Core primary amino acid sequence | NO |
| sAMK271 | Streptococcus_pneumoniae_ | GTDGADPRSTIICSATLSFIASYLGSAQTRCGKDN | 115 |
| | SPAR95 | KKK | |
| sAMK285 | Streptococcus_sp._ | GIDTLDYEISHQELSGKSAAGWQTAFRLTMQGR | 116 |
| | M334 | CGGVFTLSYECATPHVSCG | |
| sAMK286 | Streptococcus_sp._ | GGGWYTAFKLTLAGRCGLCFTCSYECTSNNVHC | 117 |
| | M334 | | |
| sAMK287 | Streptococcus_sp._ | GGWFTAIQLTLAGRCGNWFTGSFECTSNNVKCG | 118 |
| | M334 | | |
| sAMK293 | Rothia dentocariosa | GTAFPGWYSKVIGNRGRVCTVTVECMSVCQ | 119 |
| sAMK298 | Ruminococcus | GVGYTTYWGILPLVTKNPQICPVSENTVKCRLL | 120 |
| | flavefaciens | | |
| sAMK299 | Ruminococcus | GASTLPCAEVVVTVTGIIVKATTGFDWCPTGACT | 121 |
| | flavefaciens | HSCRF | |
| sAMK360 | Clostridium spp. | GEAVSYTLNCTHFLTILCC | 122 |
| sAMK416 | Corynebacterium_ | GTHPSTLIPISIALCPTTRCSRRC | 123 |
| | matruchotii_ATCC_14266 | | |
| sAMK417 | Gardnerella_vaginalis_ | GGDGVMHTLTHECHMNTWQFLLTCC | 124 |
| | 5-1 | | |
| sAMK418 | Rothia_dentocariosa_ | GGHGGGYSGGGYSGGGNSGGGNYCGNGCGNY | 125 |
| | M567 | NFGFGF | |
| sAMK419 | Clostridium_botulinum_ | GTFSEGTISITLSVYMGNDGKVCTWTVECQNNCS | 126 |
| | H04402_065 | HKK | |
| sAMK 421 | Myroides odoratimimus | GGGNSSKLYGSKGASCTCGNGVTCGTQQTKSGF | 127 |
| | CIP 103059 | EE | |
| sAMK687 | Lactobacillus iners | GSRWWQGVLPTVSHECRMNSFQHIFTCC | 128 |
| sAMK691 | Streptococcus pyogenes | GGKNGVFKTISHECHLNTWAFLATCCS | 129 |
| sAMK692 | Streptococcus pyogenes | GRGHGVNTISAECRWNSLQAIFTCC | 130 |
| sAMK695 | Mobiluncus mulieris | GTSIPCGTLIIATLTQCFNDTLVWGSCRLGTRACC | 131 |
| sAMK696 | Streptococcus | GMRFSTFSTNRCGNWSAFSWENC | 132 |
| | pneumoniae | | |
| sAMK702 | Lactobacillus delbrueckii | GGGAGLEDSKSFSLICIGSRVGDGNHSSHKKHHK | 133 |
| | | GKKH | |
| sAMK715 | Streptococcus agalactiae | GVTSKSLCTPGCKTGILMTCAIKTATCGCHFG | 134 |
| sAMK717 | Staphylococcus caprae | GNTSLIWCTPGCAKNL | 135 |
| sAMK720 | Myroides odoratimimus | GHVELMNADKVKCKSTSTTKSCSSTSTTSVD | 136 |
| sAMK725 | Streptococcus sanguinis | GVGSRYLCTPGSCWKWVCFTTTVK | 137 |
| sAMK731 | Streptococcus agalactiae | GAGHGVNTISAECRWNSLQAIFSCC | 138 |
| sAMK732 | Streptococcus agalactiae | GGKNGVFKTISHECHLNTWAFLATCCS | 139 |
| sAMK734 | Eubacterium sp. | GNMVIRARWTITSKCPSSIGHCC | 140 |
| sAMK740 | Dolosigranulum pigrum | GTANTYCRCYSGRHSCGRACTITAECPVFTVACC | 141 |
| sAMK916 | Streptococcus_sp._ | GWQTAFRLTMQGRCGGVFTLSYECATPHVSCG | 142 |
| | M334 | | |
| sAMK917 | Rothia aeria F0474 | GLIYGKYRDVLSGARLVTPPEVALRLVPR | 143 |
| sAMK989 | Enterococcus faecalis | GLWTGKFRDVFGGRALFQVVIYYRLVPR | 144 |
| sAMK995 | Sphingobium | GTSYGESLDATFPDGTPRGELTFSRLVPR | 145 |
| | yanoikuyae | | |
| sAMK1009 | Rothia aeria FO474 | GWLWGSYRDVYGVWHGPRTNFNGAGGSSEWR | 146 |
| | | LVPR | |
| sAMK1010 | Rothia aeria F0474 | GWYWGNRRDIYGALRYANKRLVPR | 147 |

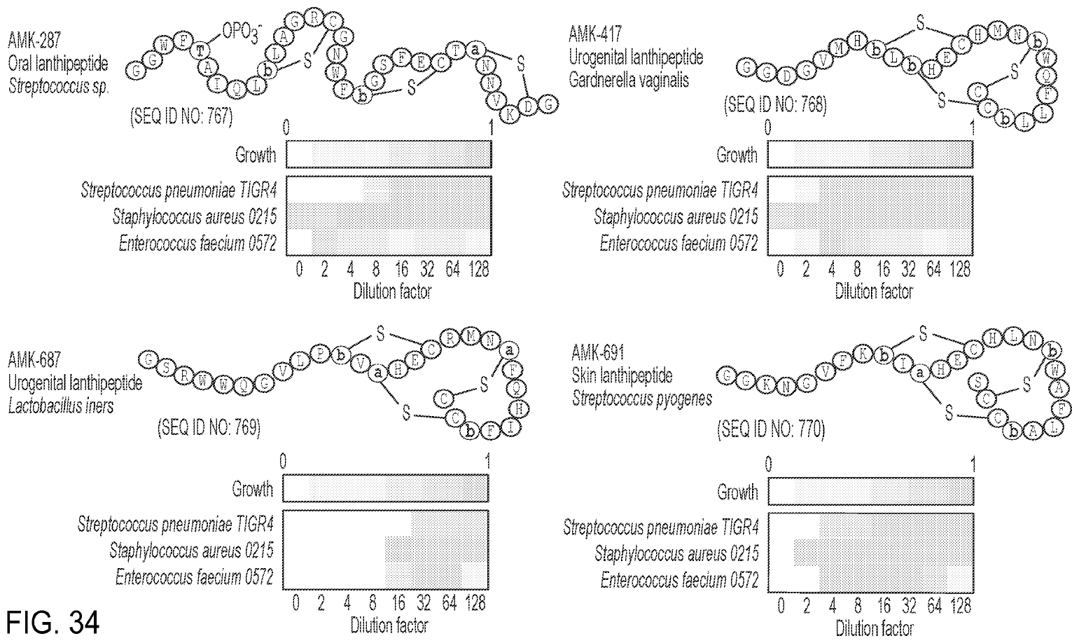

Based on the large antimicrobial activity dataset, four peptides (AMK-287, 417, 687, and 691) were selected to characterize their activity against clinical isolates of MDR pathogens. SPE-purified peptides from liter scale fermentations were used to profile dose-dependent killing of methicillin-resistant Staphylococcus aureus (MRSA), vancomycin-resistant Enterococcus faecium (VRE), and Streptococcus pneumoniae (FIG. 34). Dose-dependent antimicrobial activity was observed against at least one strain for all four peptides, with AMK-687 demonstrating particularly potent and broad-spectrum antimicrobial activity against all four isolates. Importantly, the clinical isolate of E. faecium was susceptible to these RiPPs despite a normal microbiome-associated E. faecium demonstrating robust growth in the presence of the same peptides (FIG. 33B). AMK-287 demonstrated pronounced activity against Streptococcus pneumoniae, again noteworthy given the narrow spectrum of activity against other strains of Streptococcus in the initial activity fingerprinting assay (FIG. 33B).

### DISCUSSION

Attempts to address, at a mechanistic level, the dynamics of the microbiome commonly rely on a kind of “forward genetics” approach (start with a phenotype and move toward microbial genetic determinants)¹. Here instead, a group of molecules were systematically assessed to functionally profile them for their potential to shape the microbiome. RiPPs sourced from the human microbiome may hold specific advantages as narrow spectrum antimicrobials for combating MDR pathogens. Traditional antibiotics can exacerbate the evolution of resistance or are causative of disease outright through their broad-spectrum activity disrupting the human microbiome⁵⁴. Lanthipeptides with antimicrobial activity act primarily through targeting the cell envelope⁵⁵, which is an attractive strategy to sidestep resistance mechanisms linked to enzymatic modification and efflux. Cyclic peptide natural products (or mimetics) targeting the bacterial outer envelope are being investigated and studied in clinical trials, including those active against Gram-negative pathogens⁵⁶⁻⁵⁸.

Several of the molecules discovered here serve as excellent scaffolds for further examining structure-activity relationships of the variable cyclic regions. The 96-well microtiter expression pipeline enables both rapid assessment of biosynthetic constraints for modifying enzyme/peptide pairs and functional assaying against indicator organisms of interest. Modifying enzymes that are associated with multiple substrate peptides can also serve as effective biocatalysts for selections of modified peptides with de novo activity^{25,39}. Cell-free expression approaches, as demonstrated for unmodified bacteriocins⁵⁹, offer a useful method for initial activity testing, but scalable production routes must be considered. Systematic heterologous expression and engineering of RiPP gene clusters (e.g., as provided herein) addresses the production issue and also advances peptides' potential as cell-based effectors in living therapeutics⁶⁰. Emerging technology for the delivery of genetic programs to diverse bacteria^{61,62} coupled with responsive, in situ peptide production to sidestep unfavorable pharmacokinetic properties⁶³ further highlights the therapeutic potential of peptides.

Semi-purified RiPPs were produced directly from sequence information without downstream assay constraints from as little as 2 mL microwell fermentations. Expression of RiPPs scaled well to liter volumes and methods were established for rapidly purifying and generating screening plates of peptides dissolved in an organic solvent/water mixture. These plates can be frozen, stored, and treated in similar fashion to small molecule libraries, enabling their broad assaying. The enumeration of medium-sized natural products in this format is of particular value since, compared to small molecules, they are under sampled in most natural products screening collections⁶⁴. Medium-sized modalities exhibit greater efficacy in binding 15 to and disrupting non-enzymatic function of macromolecular targets⁶⁵.

The scale at which RiPP gene clusters were constructed, expressed, and characterized in this study is unprecedented but precludes widespread, in-depth structural characterization. The application of high-resolution tandem mass spectrometry to characterize post-translationally modified peptides, however, is an acceptable level of structural annotation, as evidenced by comparable studies^{9-11, 39-45}. The workflows described here enable discovery, prioritization, and optimization of a limited number of molecules, which can be scaled in production volume for more rigorous structural and functional characterization as appropriate.

In summary, a platform was developed for streamlined genome mining of RiPP gene clusters. Rapid assessment of modification through 96-well expression, purification, and LC-MS analysis enabled small molecule and novel enzyme discovery. Application of this pipeline toward genome mining of the human microbiome yielded constrained peptides with unique antimicrobial fingerprints when tested against a large subset of strains from the human microbiome. These molecules were shown to be active against MDR bacterial pathogens. Systematic discovery and functional profiling of human microbiome-derived antimicrobials able to selectively target endogenous microflora and pathogens has significant potential for both engineering the microbiome and developing therapeutics to address antimicrobial resistance.

### Methods

### Materials and Methods

Strains, media, and chemicals. E. coli NEB 10-beta (C3019I, New England BioLabs, Ipswich, Mass., USA) was used for all routine cloning. E. coli NEB Express (C2523I, New England BioLabs, Ipswich, Mass., USA) and E. coli Marionette-X, a Marionette-compatible derivative of NEB Express were used for liter-scale peptide expression experiments. TB (T0311, Teknova, Hollister, Calif., USA) supplemented with 0.4% glycerol (BDH1172-4LP, VWR, OH, USA) was used for peptide expression and modification. 2xYT liquid media (B244020, BD, Franklin Lakes, N.J., USA) and 2xYT+2% agar (B214010, BD, Franklin Lakes, N.J., USA) plates were used for routine cloning and strain maintenance. Other media include Tryptic Soy Broth (TSB; BD211825, BD, Franklin Lakes, N.J., USA), Brain Heart Infusion (BHI; BD237500, BD, Franklin Lakes, N.J., USA),

Lactobacilli MRS broth (MRS; BD288130, BD, Franklin Lakes, N.J., USA), and Sabouraud Dextrose Broth (SDB; BD288130, BD, Franklin Lakes, N.J., USA). SOB liquid media (S0210, Teknova, Hollister, Calif., USA) was used for making competent cells. SOC liquid media (B9020S, New England BioLabs, Iwsich, Mass., USA) was used for outgrowth. Unless noted otherwise, cells were induced with the following chemicals: cuminic acid (268402, Millipore Sigma, Saint Louis, Mo., USA) added as 1000× stock (200 mM) in EtOH or DMSO; 3-oxohexanoyl-homoserine lactone (3OC6-AHL) (K3007, Millipore Sigma, Saint Louis, Mo., USA) added as a 1000× stock (1 mM) in DMSO; anhydrotetracycline (aTc) (37919, Millipore Sigma, Saint Louis, Mo., USA) added as a 1000× stock (100 μM) in DMSO; isopropyl β-D-1-thiogalactopyranoside (IPTG) (I2481C, Gold Biotechnology, Saint Louis, Mo., USA) added as 1000× stock (1 M) in water; Sodium salicylate (S3007, Millipore Sigma, Saint Louis, Mo., USA), N-(3-Hydroxytetradecanoyl)-DL-homoserine lactone (3OC14-AHL; 51481, Millipore Sigma, Saint Louis, Mo., USA. Cells were selected with the following antibiotics: carbenicillin (carb, C-103-5, Gold Biotechnology, Saint Louis, Mo., USA) added as 1000× stock (100 mg/mL in H2O); kanamycin (kan, K-120-10, Gold Biotechnology, Saint Louis, Mo., USA) as 1000× stock (50 mg/mL in H2O); and spectinomycin (spec, S-140-5, Gold Biotechnology, Saint Louis, Mo., USA). Liquid chromatography was performed with Optima Acetonitrile (A996-4, Thermo Fisher Scientific, MA, USA) and water (Milli-Q Advantage A10, Millipore Sigma, Saint Louis, Mo., USA) supplemented with LCMS Grade Formic Acid (85178, Thermo Fisher Scientific). The following solvents/chemicals were also used: Ethanol (V1001, Decon Labs, King of Prussia, Pa., USA), Methanol (3016-16, Avantor, Center Valley, Pa., USA), Ammonium bicarbonate (A6141 Millipore Sigma, Saint Louis, Mo., USA), dimethyl sulfoxide (DMSO) (32434, Alfa Aesar, Ward Hill, Mass., USA), Imidazole (IX0005, Millipore Sigma, Saint Louis, Mo., USA), sodium chloride (X190, VWR, OH, USA), sodium phosphate monobasic monohydrate (20233, USB Corporation, Cleveland, Ohio, USA), sodium phosphate dibasic anhydrous (204855000, Acros, N.J., USA), guanidine hydrochloride (50950, Millipore Sigma, Saint Louis, Mo., USA), tris (75825, Affymetrix, Cleveland, Ohio, USA), TCEP (51805-45-9, Gold Biotechnology, Saint Louis, Mo., USA), and EDTA (0.5M stock, 15694, USB Corporation, Cleveland, Ohio, USA), dimethyl formamide (A13547, Alfa Aesar, MA, USA), defibrinated sheep blood (R54012, Thermo Fisher Scientific, MA, USA), hemin (51280, Sigma Aldrich), vitamin K1 (V3501, Sigma Aldrich), and L-cysteine (C7532, Sigma Aldrich). DNA oligos and gBlocks were ordered from Integrated DNA Technologies (IDT) (San Francisco, Calif., USA).

Computational detection and clustering of RiPP gene clusters. Genome datasets for projects “HMP1” and “HMP2” were obtained from the Human Microbiome Project online portal. These 2,229 genomes were used as the database for running AntiSMASH 4.0 using default parameters with ClusterFinder-based border predictions 29. Output from this analysis was analyzed using BiG-SCAPE with distance cut-off filters of 0.2, 0.4, 0.6, 0.8, and 1.0. The resulting similarity network matrices were visualized with Cytoscape and distance cutoff of 0.8 chosen for FIGS. 28A-28D.

Peptide expression in 96-well plates and purification. Plasmids were transformed into either E. coli NEB Express or E. coli Marionette-X using 30 μL of competent cells and 1 μL of each plasmid being transformed in a PCR strip tubes (1402-4700, USA Scientific, FL, USA or 951020401, Eppendorf, N.Y., USA). Transformations were incubated on ice (20-30 min), heat shocked (42° C., 30 sec), and incubated on ice again (5 min). Cells were then transferred to a deep well 96-well plate (1896-2000, USA Scientific, FL, USA) with 120 μL of SOC. After outgrowth (Multitron Pro, 1 hr, 30° C.) in an Infors HT Multitron Pro (Infors USA, MD, USA), 900 μL LB was added with appropriate antibiotics (at 1.1× for 1× final concentration) and incubated (Multitron Pro, 30° C., 900 r.p.m.) until all wells reached saturation (12-30 hours). Overnight cultures were diluted 1:100 into 1 ml TB in deep well plates. After 3 hours incubation (Multitron Pro, 30° C., 900 r.p.m.), appropriate inducer was added (1 μl IPTG or 1l1 IPTG and 1 μl cumate), and cultures were incubated for 20 hours (Multitron Pro, 30° C., 900 r.p.m.). To purify the peptides, the 96-well plates were centrifuged (Legend XFR, 4,500 g, 4° C., 20 min), pellets were resuspended in 600 μL lysis buffer, and freeze-thawed twice (frozen at −80° C.; thawed in Multitron Pro at 37° C., 900 r.p.m). Cell lysates were centrifuged (Legend XFR, 4,500 g, 4° C., 60 min) and peptides affinity purified using His MultiTrap TALON plates (29-0005-96, GE Life Sciences, Marlborough, Mass., USA), following manufacturer instructions, using 1×600 μL water and 2×600 μL lysis buffer for column equilibration, 2×600 μL wash buffer, and 1×200 μL elution buffer.

Liter-scale RiPP expression and purification. Glycerol stocks of strains generated from 96-well transformations were used to inoculate 20 mL of LB in a 125 mL shake flask and incubated overnight at 30° C. and 250 rpm in an Innova44 (Eppendorf, N.Y., USA). A 5 mL aliquot of overnight starter culture was diluted in 500 mL total volume TB with carb/spec in Fernbach flasks and grown at 30° C. and 250 rpm until reaching OD600 0.8-1.0, at which point 1 mM IPTG and 200 μM cumate are added. Induced cultures were grown for a further 20 h at 18° C. and 250 rpm and then centrifuged (4,000 g, 4° C., 10 min) in a Sorvall RC 6+ centrifuge (Thermo Fisher Scientific, MA, USA). Pellets were resuspended in 30 mL lysis buffer (5 M guanidinium hydrochloride, 300 mM NaCl, 10 mM imidazole, 50 mM sodium phosphate, pH 7.5), and freeze-thawed twice (frozen in −80° C. freezer; thawed in innova44 incubator at 30° C., 250 rpm). Cell lysates were centrifuged (20,000 g, 12° C., 45 min) and the peptides affinity purified via gravity-flow using 3 mL resin-bed volume of Ni-NTA agarose resin (88223, Thermo Fisher Scientific, MA, USA), following manufacturer instructions, using 2 resin-bed volumes water and lysis buffer for column equilibration, 4 resin-bed volumes of wash buffer (5 M guanidinium hydrochloride, 300 mM NaCl, 25 mM imidazole, 50 mM sodium phosphate, pH 7.5), 4 resin-bed volumes of elution buffer buffer (5 M guanidinium hydrochloride, 300 mM NaCl, 250 mM imidazole, 50 mM sodium phosphate, pH 7.5). Eluates were diluted to 30 mL with lysis buffer, transferred to Spectra/Por 3 RC Dialysis Membrane Tubing 3500 Dalton MWCO (132725, Spectrum, CA, USA) and dialyzed overnight at room temperature in 1× phosphate buffered saline (PBS; 6505-4L, CalBiochem, CA, USA). Dialyzed solutions were centrifuged (4,000 g, 4° C., 10 min) to remove any precipitate. To cleave the SUMO and leader peptide from the core, TCEP (1 mM final concentration) and 3 mg of TEV protease (30 mg lyophilizate, Gene and Cell Technologies, CA, USA) were added and tubes incubated overnight at room temperature with slow orbital shaking. Cleaved peptide solutions were centrifuged (4,000 g, 4° C., 10 min) to remove any precipitate and then desalted using a Strata-X PRO 500 mg SPE tube (8B-S536-HCH, Phenomenex, CA, USA). The solid phase was first conditioned with 4 bed volumes of methanol and then water. Eluate was then loaded, washed with 8 bed volumes of water, and eluted with 8 bed volumes of 1:1 acetonitrile:water+0.1% formic acid. Solvent was removed via lyophilization at −80 C for 24-48 hours.

Liquid chromatography/mass spectrometry. All chromatography was performed using mobile phases ACN (acetonitrile supplemented with 0.1% formic acid and 0.1% water) and water (supplemented with 0.1% formic acid). LC-MS was performed on one of two mass spectrometers: “QQQ” is an Agilent 1260 Infinity liquid chromatograph with binary pump configured in low-dwell volume mode, high-performance autosampler chilled to 18° C., and column oven, coupled to an Agilent 6420 QQQ mass spectrometer equipped with an Agilent electrospray ionization (ESI) source; nitrogen gas is supplied by a Parker Nitroflowlab and ESI source parameters are 350° C. gas temp at 12 L/min flow rate, 15 psi nebulizer voltage, 4000 V capillary voltage, 135 V fragmentor voltage, and 7 V cell accelerator voltage. “qTOF” is an Agilent 1260 Infinity II liquid chromatograph with binary pump configured in low-dwell volume mode and column oven set to 40° C., coupled to an Agilent 6545 qTOF mass spectrometer equipped with an Agilent electrospray ionization (ESI) source; nitrogen gas is building supplied and ESI source parameters are 350° C. gas temperature, 12 L/min gas flow, 30 psig nebulizer pressure, 350° C. sheath gas temperature, 8 L/min sheath gas flow, 3000 V capillary voltage, 1000 V nozzle voltage, 135 V fragmentor voltage, 15 V skimmer voltage, 600 V Oct 1 RF Vpp; the mass spectrometer was run in MS mode with reference mass enabled and tuned in positive mode with standard mass range (3200 m/z) and 2 GHz extended dynamic range. When using the QQQ, analysis was done with a Phenomenex Aeris PEPTIDE XB-C18 2.6 □m 50 mm×2.1 mm column with column oven set to 40° C. Flow rate was 0.6 ml/min. Gradient was 10% ACN for 0.5 min, 10% to 60% ACN over 6 min, 60% to 90% ACN over 1 min, 90% ACN for 1 min, with 1 min re-equilibration. The mass spectrometer was run in positive mode, 500-2000 m/z range with a 300 ms scan time. Injections were 5 □L (as a starting point, injection volumes were occasionally adjusted depending on the yield of the 96-well prep). When using the qTOF, analysis was done with a Phenomenex Aeris PEPTIDE XB-C18 2.6 □m 50 mm×2.1 mm column. Flow rate was set at 0.5 ml/min. The flow rate was set at 0.5 mL/min and 5 μL sample was injected. The gradient used was 10% ACN for 1.0 min, 10% to 70% ACN over 5.0 min, 70% to 90% ACN over 0.5 minutes, 90% ACN for 1.0 min, with 1.0 min re-equilibration. Injections were 5 μL (as a starting point, injection volumes were occasionally adjusted depending on the yield of the 96-well prep).

Peptide screening plate prep. Lyophilized liter-scale preps were resuspended in 540 μL DMF and vortexed for 5 seconds. To this was added 3060 μL of H2O and the mixture was vortexed for 5 seconds to make a solution of peptide in 15% DMF. All mixtures were centrifuged (Legend XFR, 4,000 g, 4° C., 10 min) to remove any insoluble material and then split into 2 96-well 2 mL plates. From this, 12 μL of each peptide was aliquoted into 290 96-well screening plates (3788, Corning), which were then used for downstream LC-MS/MS analysis and functional assay screening. Plates were covered and kept at −20° C. for up to one year.

LC-MS/MS data acquisition. All chromatography was performed using the mobile phases ACN (acetonitrile supplemented with 0.1% formic acid and 0.1% water) and water (supplemented with 0.1% formic acid). MS/MS data were acquired on an Agilent 1260 Infinity II liquid chromatograph with binary pump configured in low-dwell volume mode and column oven set to 40° C., coupled to an Agilent 6545 qTOF mass spectrometer equipped with an Agilent electrospray ionization (ESI) source. Nitrogen gas is building-supplied and ESI source parameters are 350° C. gas temperature, 12 L/min gas flow, 30 psig nebulizer pressure, 350° C. sheath gas temperature, 8 L/min sheath gas flow, 3000 V capillary voltage, 1000 V nozzle voltage, 135 V fragmentor voltage, 15 V skimmer voltage, 600 V Oct 1 RF Vpp; the mass spectrometer was run in MS mode with reference mass enabled and tuned in positive mode with standard mass range (3200 m/z) and 2 GHz extended dynamic range. For this analysis, 4 peptide screening plates were thawed and resuspended in a total of 100 □L PBS/DMF mixture. To this mixture, TCEP was added to a final concentration of 1 mM. Samples were split in two and NEM (12.5 mM final concentration) was added to one group of samples to label free cysteine residues. For the targeted MS/MS, 4 spectra/s were sampled with fixed collision energies of 30, 45, 60, and 75 V. A narrow isolation width (1.3 m/z) and observed monoisotopic mass (exact masses found in Supplementary Fig. xx) was used for fragmentation of each peptide. Sample analysis was performed with a Phenomenex Aeris PEPTIDE XB-C18 2.6 □m 50 mm×2.1 mm column. The flow rate was set at 0.5 mL/min and 5 □L sample was injected. The gradient used was 10% ACN for 1.0 min, 10% to 70% ACN over 5.0 min, 70% to 90% ACN over 0.5 minutes, 90% ACN for 1.0 min, with 1.0 min re-equilibration. Accurate mass predictions of peptides were generated using the online resource, ChemCalc 68. Indicator strain growth. Indicator strains were grown using the annotated media. The following specialized media mixtures were used: TSB supplemented with 5% defibrinated sheep blood (TSBb) and BHI supplemented with hemin, vitamin K1, and L-cysteine (BHIs). To make BHIs, 10 mL of hemin solution (50 mg hemin, 1 mL 1 N NaOH, 100 mL H2O, filter sterilized) and 200 μL of diluted vitamin K1 solution (150 μL vitamin K1 solution, 30 mL 95% ethanol, filter sterilized) were added to sterile 1 L sterile BHI supplemented with 0.5 g L-cysteine. Agar plates of all media types were generated by addition of 2% agar. For strains sourced from OpenBiome and individual labs, strains were first purified by streaking on agar media plates. For strains sourced from ATCC and CDC, product protocols were followed to activate lyophilizates and strains were grown on agar plates of the annotated media type. All strains were grown on solid media until uniform colonies were observed. Individual colonies were used to inoculate sterile 96-well microtiter plates of the corresponding media type. Once wells reached sufficient density (24-72 hours of growth, see additional culturing conditions below), liquid glycerol stocks were generated by the addition of 500 μL culture and 500 μL 50% glycerol. Multiple glycerol stock plates were generated and frozen at −80° C. for subsequent assaying described below.

Antimicrobial assays. All materials were additionally sterilized by exposure to UV light for 10 minutes in laminar flow cabinet. Glycerol stocks of microbiome strains were subcultured in liquid media. Strains were grown for 24-48 hours, diluted 1:200 into fresh media, and 100 μL added to thawed peptide screening plates previously generated. Compounds were aliquoted in wells C1-E12 with wells B1-B12 and F1-F12 containing 15% DMF controls. Additional media was added to wells surrounding the assay wells to mitigate evaporation. All growth plates contained wells B1-B12 with a no growth control (15% DMF plus 100 μL media) and wells F1-F12 with a growth control (15% DMF plus 100 μL diluted culture). Plates were manually inspected for sufficient control growth after 24 or 48 hours and optical density measured using a The OD600 was measured using a Synergy H1 Hybrid Reader (8041000, BioTek). Automated plate shaking was found to be insufficient to break up pellets formed by some strains and therefore all pellets were manually broken up by mild pipetting with care taken to not introduce bubbles. Residual growth was calculated by measuring the OD600 of all plate wells. All measurements were done in triplicate on three separate days. Dilution series experiments were performed as above with new compound preps. Compounds were mixed with media at 4× the final concentration. Serial two-fold dilutions into the same media composition generated a compound dilution series at 2× the final assay concentration. Diluted indicator cultures were added 1:1 to this mixture to generate a 1× compound concentration in all wells.

### REFERENCES FOR EXAMPLE 6

1 Fischbach, M. A. Cell 174, 785-790, doi:10.1016/j.cell.2018.07.038 (2018).
2 Kenny, D. J. & Balskus, E. P. Chem. Soc. Rev., doi:10.1039/c7cs00664k (2017).
3 Medema, M. H. Nat. Prod. Rep. 38, 301-306, doi:10.1039/dOnp00090f (2021).
4 Zhang, J. J., et al., Nat. Prod. Rep. 36, 1313-1332, doi:10.1039/c9np00025a (2019).
5 Arnison, P. G. et al. Nat. Prod. Rep. 30, 108-160, doi:10.1039/C2NP20085F (2013).
6 Donia, M. S. et al. Cell 158, 1402-1414, doi:10.1016/j.cell.2014.08.032 (2014).
7 Montalbán-López, M. et al. Nat. Prod. Rep., doi:10.1039/dOnp00027b (2020).
8 Pinho-Ribeiro, F. A. et al. Cell 173, 1083-1097.e1022, doi:10.1016/j.cell.2018.04.006 (2018).
9 Duan, Y. et al. Nature, doi:10.1038/s41586-019-1742-x (2019).
10 Sassone-Corsi, M. et al. Nature, doi:10.1038/nature20557 (2016).
11 Nakatsuji, T. et al. Sci. Transl. Med. 9, doi:10.1126/scitranslmed.aah4680 (2017).
12 Kim, S. G. et al. Nature, 1-5, doi:10.1038/s41586-019-1501-z (2019).
13 Claesen, J. et al. Sci. Transl. Med. 12, doi:10.1126/scitranslmed.aay5445 (2020).
14 Ongey, E. L. & Neubauer, P. Microb. Cell Fact. 15, 97, doi:10.1186/s12934-016-0502-y (2016).
15 Hegemann, J. D., et al., Acc. Chem. Res. 48, 1909-1919, doi:10.1021/acs.accounts.5b00156 (2015).
16 Merwin, N. J. et al. Proc. Natl. Acad. Sci. U.S.A, doi:10.1073/pnas.1901493116 (2019).
17 Tietz, J. I. et al. Nat. Chem. Biol., doi:10.1038/nchembio.2319 (2017).
18 Santos-Aberturas, J. et al. Nucleic Acids Res. 47, 4624-4637, doi:10.1093/nar/gkz192 (2019).
19 Mohimani, H. et al. ACS Chem. Biol. 9, 1545-1551, doi:10.1021/cb500199h (2014).
20 Hudson, G. A. et al. J. Am. Chem. Soc., doi:10.1021/jacs.9b01519 (2019).
21 Schwalen, C. J., et al., J. Am. Chem. Soc., doi:10.1021/jacs.8b03896 (2018).
22 Pan, S. J., et al., Protein Expr. Purif. 71, 200-206, doi:10.1016/j.pep.2009.12.010 (2010).
23 Tran, H. L. et al. J. Am. Chem. Soc., doi:10.1021/jacs.6b10792 (2017).
24 Schmitt, S. et al. Nat. Chem. Biol., 1, doi:10.1038/s41589-019-0250-5 (2019).
25 Yang, X. et al. Nat. Chem. Biol. 14, 375-380, doi:10.1038/s41589-018-0008-5 (2018).
26 Si, T. et al. J. Am. Chem. Soc., doi:10.1021/jacs.8b05544 (2018).
27 Hetrick, K. J., et al., ACS Cent Sci 4, 458-467, doi:10.1021/acscentsci.7b00581 (2018).
28 Urban, J. H. et al. Nat. Commun. 8, 1500, doi:10.1038/s41467-017-01413-7 (2017).
29 Blin, K. et al. Nucleic Acids Res., doi:10.1093/nar/gkx319 (2017).
30 Lloyd-Price, J. et al. Nature 550, 61-66, doi:10.1038/nature23889 (2017).
31 Navarro-Munoz, J. C. et al. Nat. Chem. Biol. 16, 60-68, doi:10.1038/s41589-019-0400-9 (2020).
32 Cimermancic, P. et al. Cell 158, 412-421, doi:10.1016/j.cell.2014.06.034 (2014).
33 Maglangit, F., et al., Nat Prod Rep 38, 782-821, doi:10.1039/dOnp00061b (2021).
34 Potter, L. R. Pharmacol Ther 130, 71-82, doi:10.1016/j.pharmthera.2010.12.005 (2011).
35 Funk, M. A. & van der Donk, Acc. Chem. Res. 50, 1577-1586, doi:10.1021/acs.accounts.7b00175 (2017).
36 Meyer, A. J., et al., Nat. Chem. Biol., doi:10.1038/s41589-018-0168-3 (2018).
37 Glassey, E., et al., ACS Synth. Biol. (2020).
38 Zhang, Q. et al. Proc. Natl. Acad. Sci. U.S.A 111, 12031-12036, doi:10.1073/pnas.1406418111 (2014).
39 Hegemann, J. D., et al., ACS Synth. Biol., doi:10.1021/acssynbio.9b00080 (2019).
40 DiCaprio, A. J., et al., J. Am. Chem. Soc. 141, 290-297, doi:10.1021/jacs.8b09928 (2019).
41 Bosch, N. M. et al. Angew Chem Int Ed Engl 59, 11763-11768, doi:10.1002/anie.201916321 (2020).
42 Huo, L., Appl. Environ. Microbiol. 83, doi:10.1128/AEM.02698-16 (2017).
43 McClerren, A. L. et al. Proc. Natl. Acad. Sci. U.S.A 103, 17243-17248, doi:10.1073/pnas.0606088103 (2006).
44 Zong, C., et al., Chem. Commun. 54, 1339-1342, doi:10.1039/c7cc08620b (2018).
45 Bobeica, S. C. & van der Donk, W. A. Methods Enzymol. 604, 165-203, doi:10.1016/bs.mie.2018.01.038 (2018).
46 Spinelli, S. L., et al., J Biol Chem 274, 2637-2644, doi:10.1074/jbc.274.5.2637 (1999).
47 Ortega, M. A. et al. Nature 517, 509-512, doi:10.1038/nature13888 (2014).
48 Hudson, G. A., et al., J. Am. Chem. Soc. 137, 16012-16015, doi:10.1021/jacs.5b10194 (2015).
49 Li, B. et al. Int J Oral Sci 11, 10, doi:10.1038/s41368-018-0043-9 (2019).
50 Duranti, S. et al. Sci Rep 10, 14112, doi:10.1038/s41598-020-70986-z (2020).
51 Xu, P. et al. J Bacteriol 189, 3166-3175, doi:10.1128/JB.01808-06 (2007).
52 Diop, K., et al., Human Microbiome Journal 11, 100051, doi:10.1016/j.humic.2018.11.002 (2019).
53 Petrova, M. I., et al., Trends Microbiol. 25, 182-191, doi:10.1016/j.tim.2016.11.007 (2017).
54 Abt, M. C., et al., Nature Publishing Group 14, 609-620, doi:10.1038/nrmicro.2016.108 (2016).
55 McAuliffe, O., et al., FEMS Microbiol. Rev. 25, 285-308, doi:10.1111/j.1574-6976.2001.tb00579.x (2001).
56 Imai, Y. et al. Nature, doi:10.1038/s41586-019-1791-1 (2019).
57 Maffioli, S. I., et al., J. Ind. Microbiol. Biotechnol. 43, 177-184, doi:10.1007/s10295-015-1703-9 (2016).
58 MacNair, C. R., et al., Ann. N. Y. Acad. Sci., doi:10.1111/nyas.14280 (2019).
59 Gabant, P. & Borrero, J. Front Bioeng Biotechnol 7, 213, doi:10.3389/fbioe.2019.00213 (2019).
60 Riglar, D. T. & Silver, P. A. Nat. Rev. Microbiol. 16, 214-225, doi:10.1038/nrmicro.2017.172 (2018).
61 Brophy, J. A. N. et al. Nat Microbiol, doi:10.1038/s41564-018-0216-5 (2018).
62 Ronda, C., et al., Nat. Methods 16, 167-170, doi:10.1038/s41592-018-0301-y (2019).
63 LaMarche, M. J. et al. J. Med. Chem. 59, 6920-6928, doi:10.1021/acs.jmedchem.6b00726 (2016).
64 Harvey, A. L., Nat. Rev. Drug Discov. 14, 111-129, doi:10.1038/nrd4510 (2015).
65 Valeur, E. et al. Angew. Chem. Int. Ed Engl., doi:10.1002/anie.201611914 (2017).
66 Piewngam, P. et al. Nature, 1, doi:10.1038/s41586-018-0616-y (2018).
67 Huerta-Cepas, J., et al., Mol Biol Evol 33, 1635-1638, doi:10.1093/molbev/msw046 (2016).
68 Patiny, L. & Borel, A. J Chem Inf Model 53, 1223-1228, doi:10.1021/ci300563h (2013).

### Example 7: Combining Enzymes to Multiply Modify Peptides

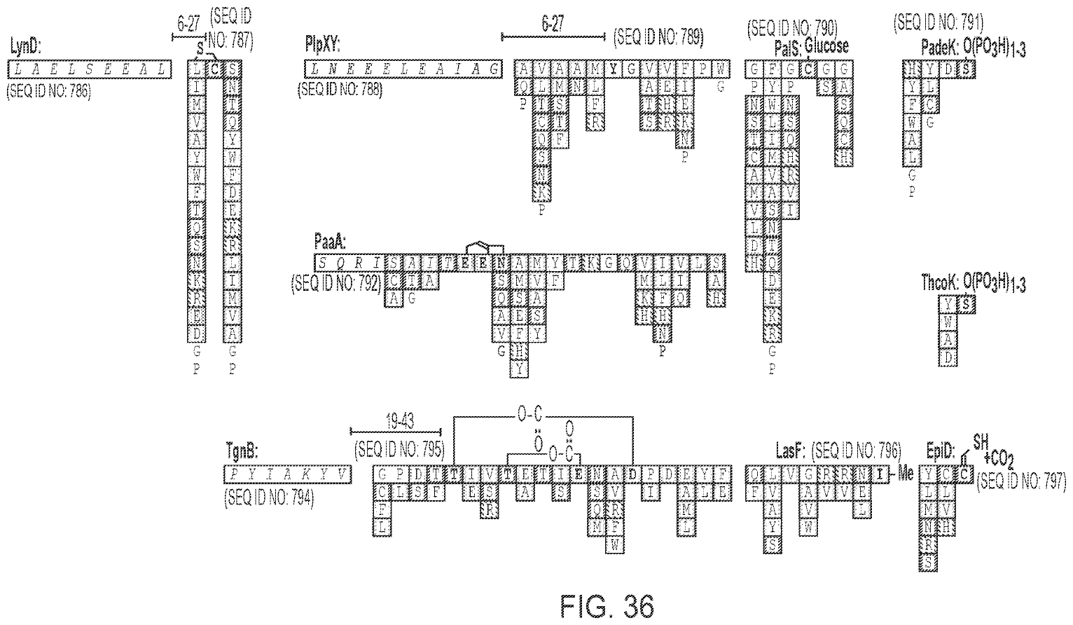

Combining peptide sequence constraints for peptide-modifying enzymes, such as those identified through methods described in the previous examples and shown in FIG. 36, offers the attractive possibility of incorporating multiple distinct modifications into a peptide library, increasing diversity of peptides that can be generated and the flexibility of options available for designing features into a library.

As a proof of principle, the modification patterns of three enzymes were combined and analyzed to develop core and leader sequence motifs. As shown in FIG. 37A, incorporating multiple distinct recognition sites (RSs) into the leader sequence of a peptide and utilizing the corresponding enzymes, as well as incorporating design limitations based on tailoring enzymes, can allow the incorporation of combinations of modifications into a peptide that are not constrained by a given native BGC. As an example, combining the design rules for LynD, PlpXY, and ThcoK modifying enzymes allows for the generation of an amino acid sequence motif that is able to be modified by three distinct enzymes, a library of such peptides can be produced and combined with the respective modification enzymes, and candidate peptides for a given function (e.g., binding to a particular target protein) can be identified (FIG. 37B). The core sequence constraints identified for these three enzymes were combined to design a motif of allowable amino acids forming the range of options for core sequences that can be modified by the particular combination of three enzymes (FIG. 37C). Similarly, the recognition sites for the two leader-dependent enzymes were analyzed, and an array of chimeric leaders were generated by varying the positioning and overlap of the two RSs and scoring each output (FIG. 37D).

After analyzing the core and leader sequence variant possibilities, a chimeric leader and hybrid core motif were identified combining the options for LynD, PlpXY, and ThcoK modifications (FIG. 37E). The resulting motif provides 11,520 possible peptide sequences. This informed design strategy enables the generation of libraries of this order of magnitude which can be screened, whereas absent the incorporation of design limitations based on the selected modifying enzymes, screening a library of unconstrained peptides of the same length would be infeasible, as there would be 20¹³ or roughly 10¹⁶ possible options—a library which would be impossible to screen using any available techniques. To the contrary, the rationally designed library of ˜10⁴ peptides was able to be screened using the methods provided here, thereby enabling the identification of candidates with the desired modification patterns. The hybrid motif was encoded as a degenerate library, and 11 library members were isolated (FIG. 37F). Certain of the library members included amino acids that theoretically were not allowed based on the hybrid motif (shaded in FIG. 37F), as a result of the degenerate codons that were used to encode certain amino acid positions.

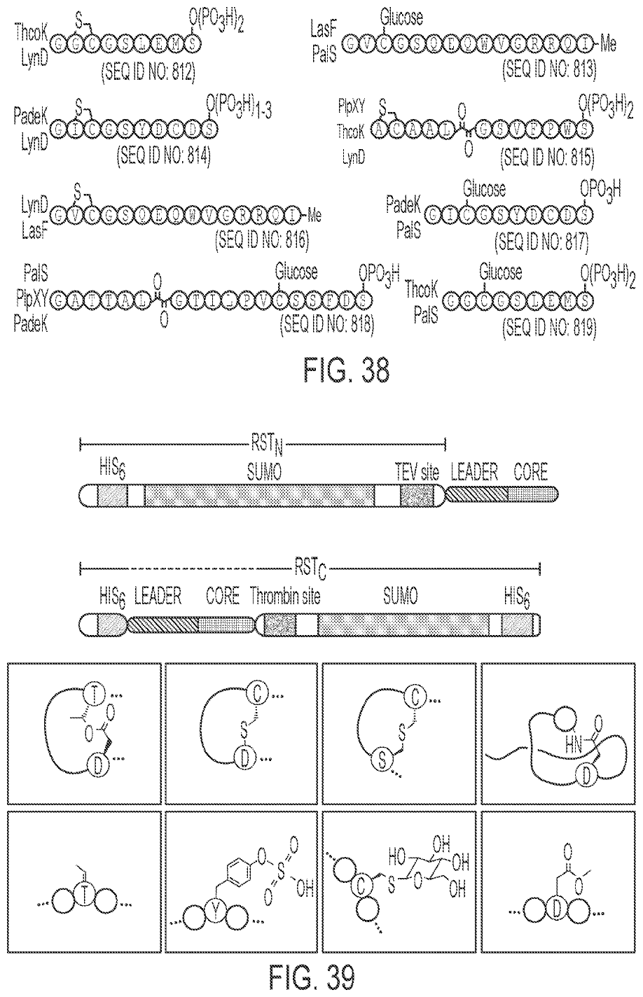

Similar methods were applied to additional combinations of modification enzymes: (a) ThcoK and LynD; (b) PadeK and LynD; (c) LynD and LasF; (d) PalS, PlpXY and PadeK; (e) LasF and PalS; (f) PlpXY, ThcoK and LynD; (g) PadeK and PalS; and (h) ThcoK and PalS. A selection of peptides were identified for these combinations (FIG. 38).

### Example 8: Functional Expression of Diverse Post-Translational Peptide-Modifying Enzymes in E. coli

RiPPs (ribosomally-synthesized and post-translationally modified peptides) are a class of pharmaceutically-relevant natural products expressed as precursor peptides before being enzymatically processed into their final functional forms. Bioinformatic methods have illuminated hundreds of thousands of RiPP enzymes in sequence databases and the number of characterized chemical modifications is growing rapidly; however, it has proven difficult to functionally express them in a heterologous host. A major challenge is peptide stability, which is addressed in this Example by design of a RiPP stabilization tag (RST) based on a small ubiquitin-like modifier (SUMO) domain that can be fused to the N- or C-terminus of the precursor peptide and proteolytically removed after modification. This is demonstrated to stabilize a set of eight RiPPs representative of diverse phyla without interfering with the activity of associated modifying enzymes. Further, using Escherichia coli for heterologous expression, a common set of media and growth conditions were identified in which 24 modifying enzymes, representative of diverse chemistries, were shown to be functional. The high success rate and broad applicability of this system enables RiPP discovery through high-throughput “mining” as well as retrosynthesis through the artificial combination of enzymes from different pathways to create a desired non-natural peptide.

### INTRODUCTION

Metagenomics has led to a deluge of microbial genomes, leading to high-throughput efforts to “mine” the molecules made by organisms by rebuilding pathways and screening for functions-of-interest[1-3]. Because these genes are gleaned from sequence databases, the organism or genomic DNA may not be available, thus necessitating the use of DNA synthesis and a heterologous host to obtain the chemical product[4-6]. RiPPs (ribosomally-synthesized and post-translationally modified peptides) are a potentially rich source of functional diversity that are encoded in gene clusters as a precursor peptide that is enzymatically modified before being proteolytically released[7-14]. Because the peptidic product is made by the ribosome, rather than by a large megasynthase, the probability of successful heterologous expression was determined to be high. However, expressed peptides are often unstable in vivo, and post-translational modifying enzymes may not function in new contexts[15-17]. As a result, only a small fraction of the thousands of known RiPP pathways have been explored[13].

RiPPs are classified by the chemical modifications made to the peptide. Some are defined by cyclization chemistry, including lanthipeptides (lanthionine macrocyclizations), thiopeptides ((4+2) cycloaddition of dehydrated serine/threonine), lasso peptides (N-terminal macrocyclization with asp/glu), graspetides (lactone/lactam macrocyclizations), bottromycin (macrolactamidine macrocyclization), ranthipeptides (Non-Cα thioether macrocyclizations), pantocins (glutamate crosslink), and sactipeptides (sactionine macrocyclizations)[7, 14]. Others are defined by individual modifications, like glycocins (side chain glycosylation), microcin C (aminoacyl adenylation or cytidylation), comX (indole cyclization and prenylation), sulfatyrotide (tyrosine sulfation), spliceotide (β-amino acids from backbone splicing), and cyanobactins (N-terminal proteolysis). Precursor peptide organization varies between RiPP classes. Modifying enzymes can either bind to a leader/follower sequence in the precursor peptide or directly modify the core. The core consists of 2 to over 50 amino acids and there can be multiple cores in one precursor peptide[17-20]. Leader peptides range from 7 to over 80 amino acids and can recruit multiple modifying enzymes that can have overlapping binding sequences[21-23]. The diversity in chemistry and genetic encoding complicates the creation of general engineering tools that can be systematically used for mining efforts across RiPP classes.

Tools have been developed to aid heterologous production, including multi-plasmid inducible systems and exploration of E. coli, various Streptomyces strains, and Microvirgula aerodentrificans as expression hosts[17, 30-33]. In vitro methods have also been used to engineer production of new molecules or study biosynthesis[34-37]. Gene cluster regulation may not function properly in a new host. To overcome this, the precursor peptide and modifying enzymes can be cloned and expressed separately[17, 33]. However, precursor peptides have been observed to often be unstable due to host proteases, thus necessitating the use of stabilization tags[15, 16, 24]. Large tags must be removed before peptide modifications can be observed by mass spectrometry, such as in the case of maltose binding protein (MBP, 45 kD), green fluorescent protein (GFP, 27 kD) and glutathione-S-transferase (GST, 26 kD)[38]. In contrast, the small ubiquitin-like modifier tag (SUMO, 12 kD) is smaller, thus allowing modifications to be observed prior to its removal. Further, it can be removed using SUMO protease immediately after purification without desalting[39], which simplifies its use in high-throughput formats. SUMO has been used for expression of both eukaryotic and prokaryotic antimicrobial peptides in E. coli [40-42] as well as a post-translationally modified lanthipeptide from Lactococcus[43] and a xenorceptide from Xenorhabdus[44].

Here, a RiPP Stabilization Tag (RST) was developed. The RST is a SUMO-based tag for high-throughput RiPP production and was demonstrated to work with diverse classes and modifying enzymes. Versions were made for fusion to the N- or C-terminus of the precursor peptide. Each version contains a HIS6 tag to enable purification in 96-well format. TEV and thrombin protease cleavage sites were included for the N- and C-terminal versions, respectively. Optimized E. coli inducible systems[45] were used to express tagged precursor peptides and modifying enzymes from separate plasmids. The ability for the RST to stabilize the peptide was validated by testing precursor peptides from 9 RiPP classes. As an example, it was demonstrated that the B. halodurans antibiotic peptide haloduracin A1/A2 can be expressed in E. coli and completely modified while attached to the RST, and further that the peptide is functional upon proteolytic cleavage of the RST. Fifty (50) precursor peptides were tested with 47 modifying enzymes and 39 peptides were identified that were expressed as RST fusions, and 24 were identified that were able to be modified with the RST attached. This Example demonstrates the broad applicability of the RST tag for high-throughput mining efforts that span RiPP classes and modifying enzyme chemistries. In addition, these enzymes were all expressed in the same heterologous host (E. coli) under uniform culture conditions and induction times. This provides a roadmap for selecting those enzymes that can be artificially combined to build retrosynthetic pathways for producing non-natural RiPP molecules with desired properties.

### Results

### Expression System for Modified Peptides

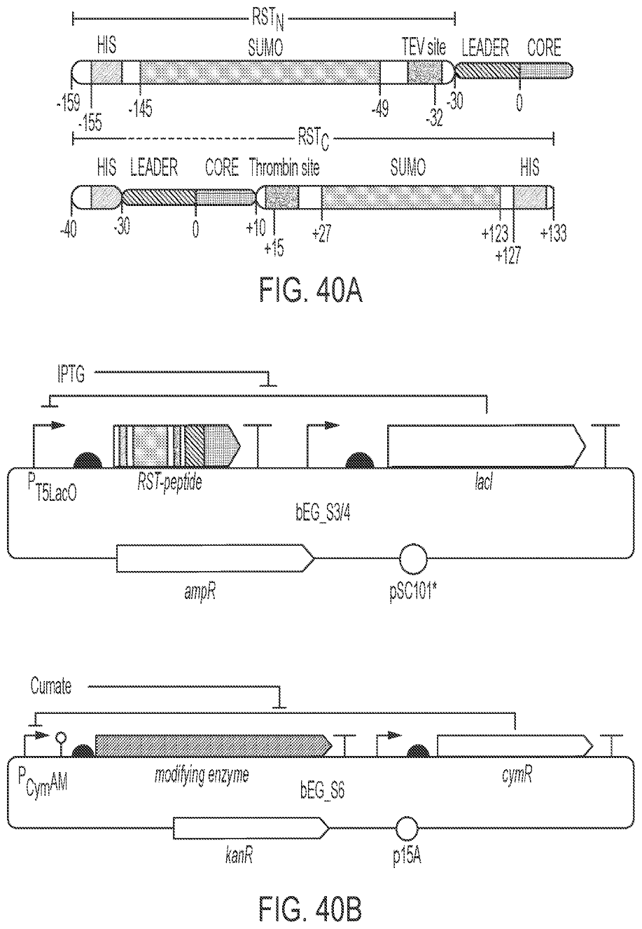

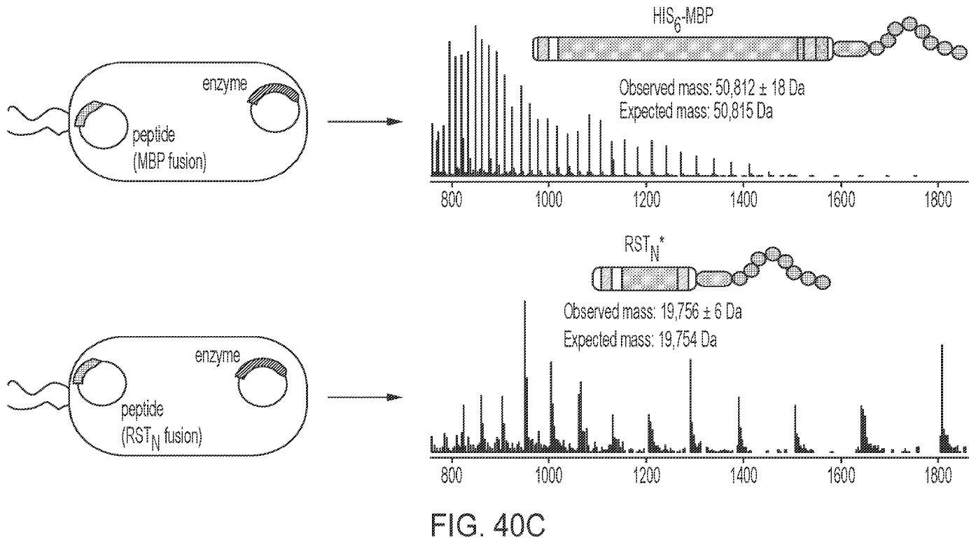

Two versions of the RiPP stabilization tag (RST) were designed to allow fusion to either the N- or C-terminus (termed RST_{N} and RST_{C}, respectively) of a precursor peptide (FIG. 39 and FIG. 40A). In most instances in this Example, the N-terminal version was used. The C-terminal version is a useful alternative either when there is a modification at the N-terminus, or when the leader peptide is removed during modification. For purification, a HIS6 tag was placed at the terminus of the RST. A linker sequence was designed to connect SUMO to the precursor peptide, adapted from a recombinant protein expression system[46]. The linkers were built to include cleavage sites for orthogonal proteases: TEV (for RST_{N}) or Thrombin (for RST_{C}). The RST was designed such that it can be removed using either TEV/Thrombin or SUMO protease (for RST_{N}). Treatment of RST_{N}-tagged peptides with TEV leaves a GC scar at the N-terminus, where the cysteine is included to allow SAMDI (self-assembled monolayers on gold for matrix-assisted laser desorption/ionization) mass spectroscopy[47, 48].

A two-plasmid system was used to separately express the precursor peptide and modifying enzyme, thus enabling combinations to be tested rapidly through co-transformations (FIG. 40B). The inducible system for the precursor peptide was selected to maximize its expression. To this end, the IPTG-inducible PT5LacO promoter[45] was used and a strong ribosome binding site (RBS) designed using the RBS Calculator[49, 50]. For the modifying enzyme, the cumate-inducible P_{CymR*} or ahl-inducible P_{LuxB} plasmids were used because of their high dynamic range (low off and high on)[45]. A different RBS was calculated for each modifying enzyme to maximize the probability of successful expression and to bias toward similar expression levels. When expressing RSTC-fused peptide, a small N-terminal region of the RSTN tag (FIG. 40A) was used to keep the RBS strength (and associated expression level) relatively constant across different precursor peptides.

Expression and purification protocols were first developed for low-throughput growth in 250 ml flasks in LB media. The tagged precursor peptide and modifying enzyme were induced simultaneously. After induction with 1 mM IPTG and 200 μM cumate (for P_{CymR*}) or 10 μM 3OC6-AHL (for P_{LuxB}), cultures were grown at 18° C. for 20 hours with shaking. Then, the peptide was purified using immobilized metal affinity chromatography (IMAC) and analyzed using LC-MS.

An example of the production of a modified peptide in flasks is shown in FIG. 40C using a variant of the trunkamide precursor peptide (TruE*) and cognate modifying enzyme TruD. Two samples were prepared: (1) the TruE* peptide expressed using a first-generation version of RST_{N} (RST_{N}*) co-transformed with the plasmid containing P_{LuxB}-controlled truD (pEG1128); and (2) the TruE* peptide expressed as an MBP fusion, also co-transformed with pEG1128. From the LC-MS spectra, the observed mass for each of the peptides, as well as the expected error given the resolution of the mass spectrometer were calculated. TruD catalyzes the formation of a thiazole from cysteine, causing a loss of water and a corresponding mass shift of −18 Da. The larger MBP obfuscated the observation of this expected mass shift because it is equal to the standard deviation of the measurement (18 Da). In contrast, the standard deviation of the RST_{N} fusion is 6 Da and the expected and observed mass matched (FIG. 40C). Therefore, it was concluded that the mass shift that occurs due to post-translational modification could be observed without removing the RST, even using a low-resolution quadrupole mass spectrometer.

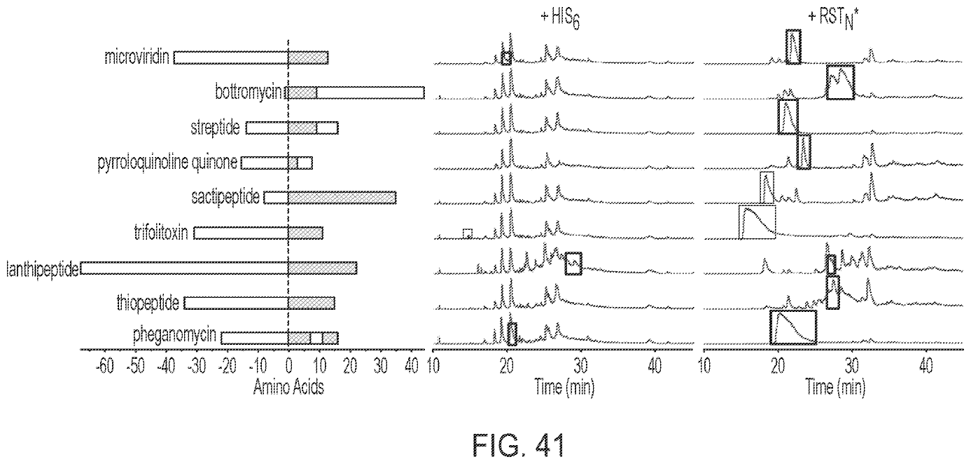

Next, RST stabilization of diverse precursor peptides across RiPP classes was tested (FIG. 41). The following examples from each class were selected: microviridin L from graspetides, bottromycin from bottromycins, streptide from streptides, PQQ from pyrroloquinoline quinones, subtilosin A from sactipeptides, trifolitoxin from linear azole peptides, prochlorosin from lanthipeptides, thiomuracin from thiopeptides, and pheganomycin from guanidinotides (peptides described in [14, 20, 29, 51-57]). This set encompasses a wide range of lengths, amino acid compositions, number of modifying enzyme binding sites, N- and C-terminal leaders/followers, and pheganomycin has two cores.

The ability for RST_{N}* to stabilize the unmodified peptides was tested. Expression was measured in the absence of modifying enzymes to account for any stabilization affect that arises from peptide modification. Expression and purification were performed at the 250 mL flask scale, as described above. First, precursor peptide expression when fused only to a N-terminal HIS6 tag was evaluated. This tag led to only three of nine peptides being detected by LC-MS (FIG. 41). Trifolitoxin was also detected, but it was cleaved in E. coli, resulting in a truncated peptide. In contrast, when the precursor peptides were fused to RST_{N}*, large peaks appeared for all of the peptides. These peaks corresponded to the expected masses, except for trifolitoxin and subtilosin A, the latter of which is cleaved in the leader.

### Production of Active Haloduracin

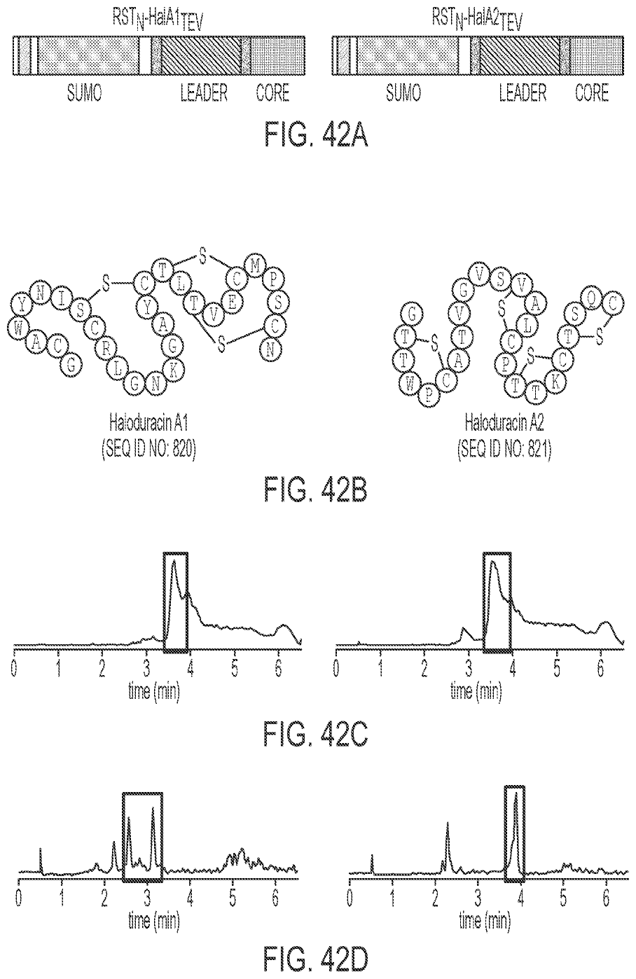

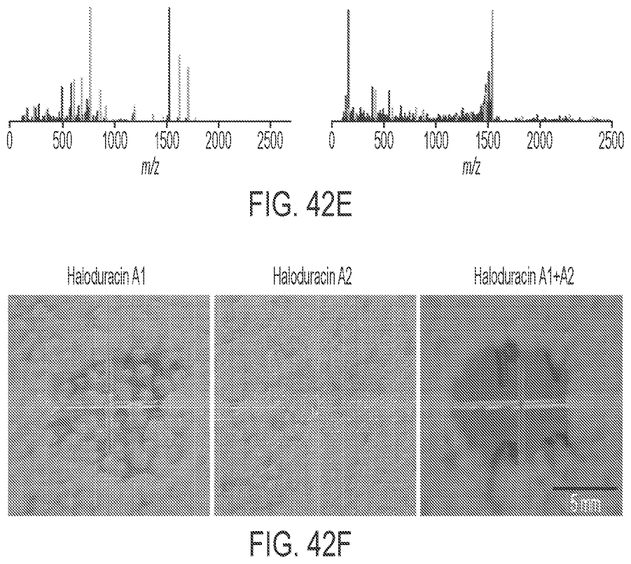

Next, the production of a biologically-active product was evaluated using the expression system provided herein. Modifications were directed at an RST-fused peptide, after which the tag was cleaved and the activity of the product tested. Haloduracin was selected, a two-component lanthipeptide that had previously been expressed and purified from E. coli and shown to have antibiotic activity[34]. Genes encoding haloduracin A1 and haloduracin A2 peptides fused to RST_{N} were synthesized, as were genes encoding corresponding HalM1 and HalM2 modifying enzymes from Bacillus subtilis (FIGS. 42A-42F). An additional TEV protease cleavage site was added between the leader and core regions of the precursor peptide (FIG. 42A) to allow the core to be cleaved and recovered as an active product (FIG. 42B). Such cleavage leaves a single N-terminal glycine on the released core sequence. The peptide-enzyme genes were cloned into the two-plasmid system (FIG. 40B) and transformed as pairs into E. coli NEB Express.

A high-throughput 96-well system for expression and purification was developed, which was tested using haloduracin. Cultures were grown in 2 mL of TB media in deep well plates (two 1 mL wells for each peptide), where they are each induced with 1 mM IPTG/200 μM cumate for 20 hours at 30° C. with shaking. The cells were lysed, affinity-purified and desalted using solid phase extraction, all in 96-well format. Then, the samples were treated with TEV protease to remove RST_{N} and the leader peptide, and desalted again to concentrate the core peptide (FIG. 42D). The presence of the cleaved cores was verified by LC-MS (FIG. 42D) and LC-MS/MS to confirm that SUMO did not disrupt or alter the lanthionine macrocyclizations present in both molecules (FIG. 42E). For both, the predicted structures were in close agreement with previous reports ([34, 58, 59]). For HalA2, seven of eight Ser/Thr residues were dehydrated and assignment of the single unmodified residue was previously localized to Thr18, Thr22, or Ser23 [34, 58, 59]. A low abundance fragment was observed, suggesting the presence of a dehydrated Ser23, in contrast to a previous report wherein mutation of Ser23 to Ala did not affect the overall number of dehydrations observed [58].

To assay for antimicrobial activity, the cleaved and desalted core peptides were resuspended in 50 μL 1:1 methanol:water. Bacillus subtilis PY79 was used as indicator strain and was spread on a LB-agar surface, on which 5 μL of either or both haloduracins or a solvent control was added. Individually, the haloduracin peptides showed limited activity (FIG. 42F, left two panels), but combined they formed a clear halo of growth inhibition (FIG. 42F, rightmost panel), indicating that both peptides were properly modified and cyclized. The solvent control showed no effect on bacterial growth.

### High-Throughput Assay of Diverse Modifying Enzymes

A set of 47 modifying enzymes and their cognate 50 precursor peptides was collated from the literature. The complete list of pathways and enzymes is provided in Table 13 and Table 14, and the subset ultimately found to be active in this Example is provided in Table 15. The selected modifying enzymes are representative of 13 bacterial RiPP classes from diverse genera and catalyze 22 different chemical transformations, including glycosylation, radical carbon-carbon bond focpation and cysteine heterocyclization. The precursor peptide and modifying enzyme genes were codon optimized for E. coli and synthesized, or amplified when the source DNA was available, and cloned into the two-plasmid system. The precursor peptides were tagged with RST_{N}, except for macrocyclization of lasso peptides, which were fused to RST_{C}. The plasmids containing the modifying enzymes and precursor peptides were co-transformed into E. coli NEB Express.

TABLE 13
Modification enzymes

| | Cluster | | | |
|---|---|---|---|---|
| RiPP Class | Name | Molecule Name(s) | Producing organism | Biological Activity |
| Lasso-peptide | Las | lassomycin | Lentzea kentuckyensis | Antibiotic |
| | Cap | capistruin | Burkholderia thailandensis E264 | Antibiotic |
| | Albs^{a} | albusnodin | Streptomyces albus | Unknown |
| | Atx | astexin 1-3 | Asticcacaulis excentricus | Unknown |
| | Cln | caulonodin I-VII | Caulobacter sp. K31 | Unknown |
| | Cseg | caulosegnins I-III | Caulobacter segnis | Unknown |
| | Pade | Paeninodin | Paenibacillus dendritiformis C454 | Unknown |
| | Thco | unnamed | Thermobacillus composti KWC4 | Unknown |
| | Papo | unnamed | Paenibacillus polymyxa CR1 | Unknown |
| | Stsp | unnamed | Streptomyces sp. Amel2xC10 | Unknown |
| Glycocin | Lcn | listeriocytocin | Listeria monocytogenes SLCC2540 | Unknown |
| | Pal | pallidocin | Aeribacillus pallidus 8 | Antibiotic |
| Microcin C | Bam | unnamed | Bacillus amyloliquefaciens DSM7 | Antibiotic |
| ComX | Com | ComX | Bacillus subtilus | quorum sensing |
| Pantocin | Paa | pantocin | Pantoea agglomerans | Antibiotic |
| Sulfa-tyrotide | Rax | RaxX | Xanthomonas oryzae | Plant signaling |
| Splice-otide | Plp | unnamed | Pleurocapsa sp. PCC7319 | Unknown |
| | Pcp | unnamed | Pleurocapsa sp. PCC7327 | Unknown |
| Lanthi-peptide | Crn | carnolysin A1′ | Carnobacterium maltaromaticum C2 | Antibiotic |
| | | carnolysin A2′ | | |
| | Sgb | unnamed | S. globisporus subsp. globisporus | Unknown |
| | | | NRRL B2293 | |
| | Bsj | bicereucins | Bacillus cereus SJ1 | Antibiotic |
| | Ltn | lacticin S | Lactococcus lactis | Antibiotic |
| | | lacticin 3147 | | |
| | Proc | prochlorosins | Prochlorococcus MIT9313 | Unknown |
| | Mcb | microcin B17 | Escherichia coli | Antibiotic |
| | Mib | micro-bisporicin | Microbispora corallina | Antibiotic |
| | Cin | cinnamycin | Streptomyces cinnamoneus | Antibiotic |
| | | | cinnamoneus DSM 40005 | |
| | Hal | haloduracin A1 | Bacillus halodurans C-125 | Antibiotic |
| | | haloduracin A2 | | |
| | Epi | epidermin | Staphylococcus epidermidis | Antibiotic |
| Micro-viridin | AMdn | unnamed | Anabaena sp. PCC7120 | Unknown |
| | Psn | plesiocin | Plesiocystis pacifica | protease inhibitor |
| | Mdn | microviridin L | Microcystis aeruginosa NIES843 | protease inhibitor |
| | Tgn | unnamed | Bacillus thuringiensis serovar | Unknown |
| | | | huazhongensis BGSC 4BD | |
| Cyano-bactin | Tru | trunkamide | Prochloron spp. | Unknown |
| | | patellins | | |
| | Lyn | unnamed | Prochloron spp. | Unknown |
| | Kgp | kawaguchi-peptin | Microcystis aeruginosa NIES-88 | Unknown |
| Thio-peptide | Pbt | GE2270 | Planobispora rosea | Antibiotic |
| Sacti-peptide | Alb/Sbo | subtilosin A | Bacillus subtilis subsp. spizizenii | Antibiotic |
| | Pap | freyrasin | Paenibacillus polymyxa ATCC 842 | Antibiotic |

TABLE 14
Enzyme-mediated modifications

| Peptide Type | Enzyme Type | Mass Shift^{a} | Enzyme Name |
|---|---|---|---|
| Lassopeptide | Amino- | −Leader (leader | LasBCD, CapBC, AlbsBC, |
| | peptidase + cyclase | cleavage) −18 Da | AtxBC, Cln1BC, Cln2BC, |
| | | (cyclization) | Cln3BC, CsegBC |
| | Acetyl-transferase | +42 Da (acetylation) | AlbsT |
| | Kinase | +80 Da (phosphorylation) | PadeK, ThcoK, PapoK |
| | O-methyl-transferase | +14 Da (methylation) | LasF, StspM |
| Glycocin | Glycosyl-transferase | +162.14 Da (glycosylation) | LcnG, PalS |
| Microcin | cytidylyl-transferase | +305.18 Da (cytidylation) | BamB |
| ComX | Prenyl transferase | +204.4 Da (prenylation) | ComQ |
| Pantocin | Claisen | −80 Da (Claisen condensation and | PaaA |
| | | decarboxylation) | |
| Sulfatyrotide | Sulfo-transferase | +80 Da (sulfation) | RaxST |
| Spliceotide | rSAM tyrosinase | −135 Da (tyramine excision) | PlpXY, PcpXY |
| Lanthipeptide | LanM: Dehydratase + | −18 Da (dehydration) | CmM, SgbL, BsjM, LtnM1, |
| | thioether cyclase | | LtnM2, ProcM, HalM1, HalM2 |
| | TOMM | −18 Da (dehydration) | McbCD |
| | halogenase | +34.5 Da (chlorination) | MibHS |
| | P450 | +16 Da (hydroxylation) | MibO, CinX |
| | De-carboxylase | −44 Da (decarboxylation) | MibD, EpiD |
| Microviridin | Lactone cyclase | −18 Da (dehydration) | AMdnC, PsnB, MdnC, TgnB |
| Cyanobactin | TOMM | −18 Da (dehydration) | TruD, LynD |
| | Prenyl transferase | +136.2 Da (prenylation) | KgpF |
| Thiopeptide | P450 | +16 Da (hydroxylation) | PbtO |
| | N-methyl-transferase | +14 Da (methylation) | PbtM1 |
| Sactipeptide | rSAM cyclase | −2 Da (dehydrogenation) | AlbA |
| SCIFF/ | rSAM cyclase | −2 Da (dehydrogenation) | PapB |
| Ranthipeptide | | | |

^{a}Mass shift listed is for a single modification. Enzymes can multiply-modify their peptide substrate, resulting in a total mass shift that is multiplied by the integer number of modifications performed.

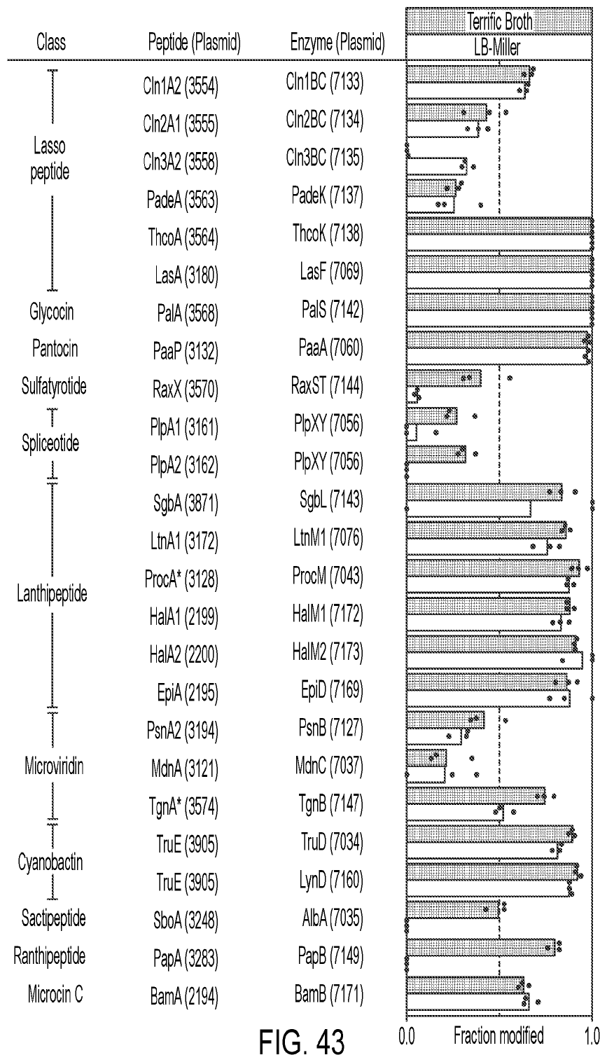

The cultures were grown following the high-throughput protocol in 96-well plates. Both TB and LB media have been used previously to functionally express certain RiPPs in E. coli. The choice of media can impact the function of an enzyme; for example, radical S-adenosyl-L-methionine (rSAM) enzymes are more active in TB than LB, the latter requiring a reduction in shake speed and/or increased iron-sulfur cluster biosynthesis [22, 60, 61]. For applications requiring the high-throughput mining or the artificial combination of RiPP enzymes (retrosynthesis), it is desirable to have a single set of culture conditions. To this end, the ability for the enzymes to modify their precursor peptides was evaluated following the same culture conditions either in LB or TB (Table 15 and FIG. 43). All of the enzymes and precursor peptides were expressed in 1 mL of media in deep-well plates with shaking. Induction by 1 mM IPTG and 200 μM cumate was performed for 20 hours at 30° C., after which the modified peptide was purified and desalted. In all cases, the modification could be observed by LC-MS without cleaving RST_{N}. In total, 24/47 (51%) of the enzymes tested were found to be active against at least one peptide in one of the medias tested. The % modified values shown in Table 14 were calculated from the extracted compound chromatograms (ECCs) based on the expected charge state m/z's for unmodified, partially modified (if relevant) and modified peptide molar masses. More enzymes (24) had activity in TB than LB (20) and, on average, the % modified was higher. As expected, rSAM enzymes (AlbA, PapB, PlpXY) were found to be more active in TB and several only had activity in this media. Similarly, RaxST is a sulfur-requiring enzyme that was found to be more active in TB.

The 25 modified peptides shown in Table 14 showed the exact mass change that was expected to result from the modification shown. However, some modifications could occur at different positions than the wild-type modification, leading to a different peptide with the same mass. In instances in which multiple modification products are possible, the addition of an RST could change where the modification occurs. To test for this outcome, several modifications were selected from different classes for evaluation by LC-MS/MS. The following were selected for structural annotation: PsnA2 macrolactonization by PsnB, and PapA sactionine macrocyclization by PapB. The precursor peptides were modified to contain a TEV cleavage site between the leader and core peptides. The modifying enzymes and precursor peptides were expressed following the high-throughput protocol, the RST and leader peptide removed using TEV protease, and the modified core analyzed with LC-MS/MS. Fragmentation of PsnA2 was observed between the core repeats, with each core repeat fragment mass corresponding to two lactone macrocyclizations per repeat, in agreement with previously published results[19]. Within each core repeat, MS/MS was not able to validate the cyclization topology within each core, which was previously determined by analyzing partially hydrolyzed modified peptide. Without using high collision energies, fragmentation products of PapA were only observed outside of predicted C-D ring structures, in agreement with published MS/MS spectra[61].

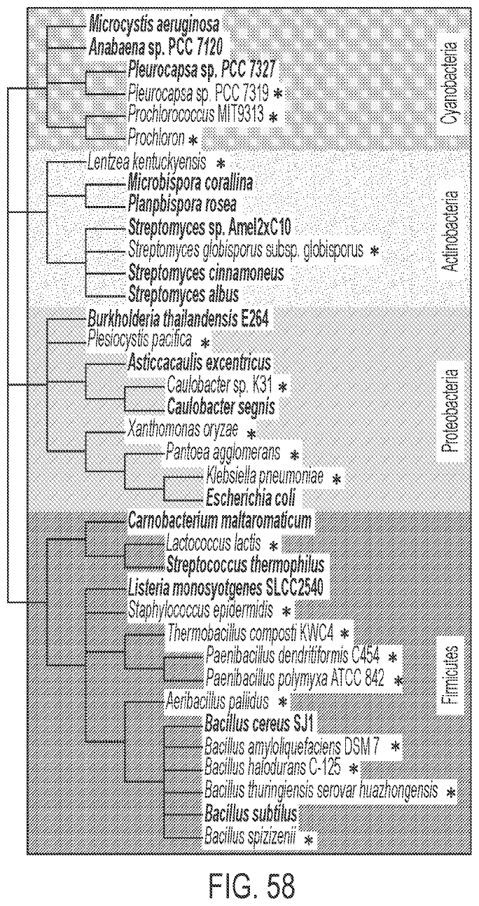

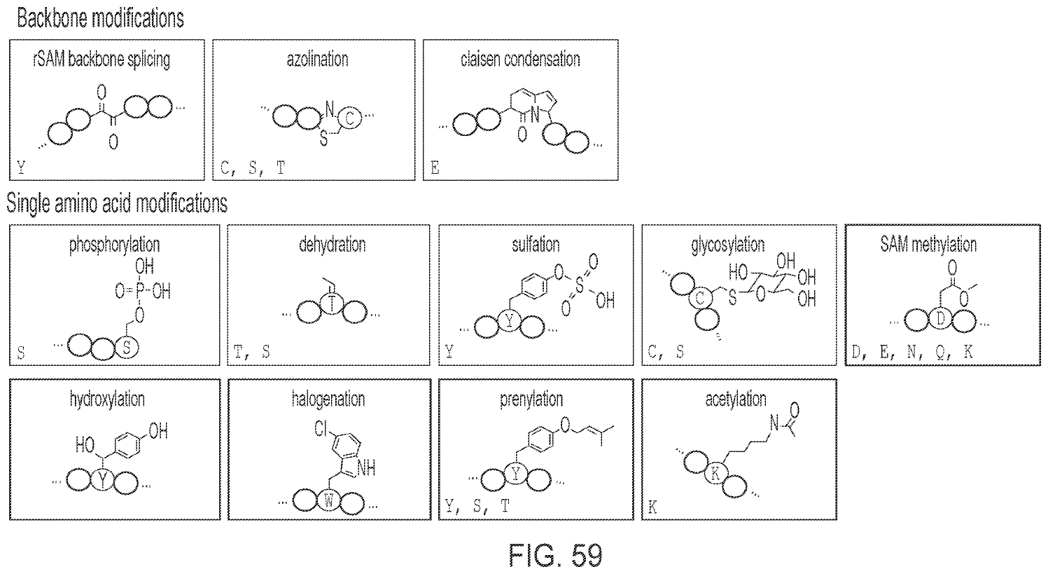

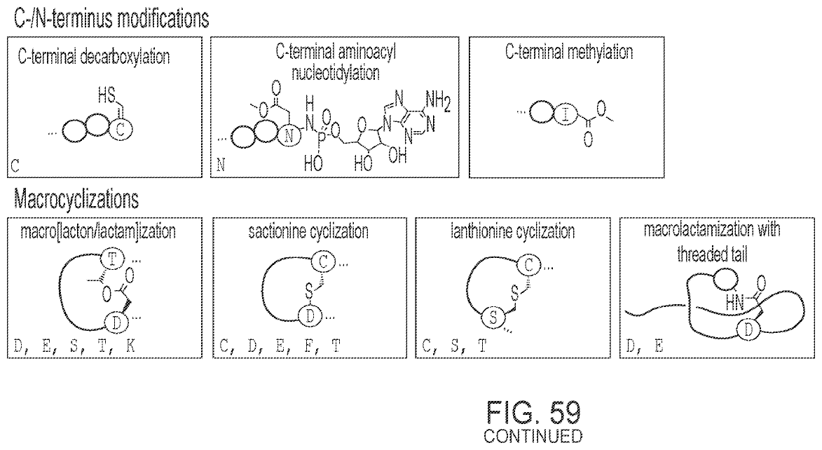

Of the enzymes tested, 23 of the 47 did not correctly modify a peptide when co-expressed in E. coli. Patterns based on the phylogeny from which the pathway was sourced were sought, noting that the sources spanned cyanobacteria, actinobacteria, proteobacteria, and firmicutes (FIG. 58). Each of these phyla provided functional examples. The least successful phylum, Actinobacteria, yielded 2/7 functional pathways, but this was not too different from the 5/9 success rate of Proteobacteria, of which E. coli is a member. Therefore, it was determined that there is no relationship between similarity to E. coli and the likelihood of success. Enzymes categorized according to most modification chemical transformation types had at least one enzyme that was functional (FIG. 59), but both prenyl transferases (ComQ and KgpF) and all three P450 oxidases (MibO, CinX, and PbtO) were not functional. For other non-functional chemical transformation types, only one example was tested (acetyl-transferase: AlbsT, halogenase: MibHS, and N-methyl transferase: PbtM1).

### DISCUSSION

While the number of characterized RiPP enzymes is growing rapidly in the literature, the conditions under which each enzyme is characterized vary across studies. This poses a challenge for high-throughput screening efforts if the conditions have to be re-optimized for each pathway. This Example presents a side-by-side survey of recombinant RiPP enzymes in E. coli, using the same growth and induction methods. Further, this Example provides protocols for every step to be performed in 96-well plate format under conditions that are consistent with high-throughput screening platforms [2, 70-72]. The RSTs address the problem of precursor peptide stability, for which degradation and solubility are the dominant causes of unobservable product. Their use increases the probability that a pathway will be successfully expressed in a new host; in other words, they increase the “hit rate” of screening efforts. The RSTs do not interfere with the action of modifying enzymes, facilitate high-throughput purification and do not need to be removed prior to LC-MS analysis of modifications. Software was developed to rapidly analyze LC-MS data. Collectively, this presents a suite of tools that enable the high-throughput screening of RiPP pathways mined from sequence databases [13, 73, 74]. In this manuscript, the action of only a single enzyme at a time was investigated. To mine complete RiPP-encoding gene clusters, additional enzyme genes can either be assembled as operons or placed under the control of different inducible promoters (e.g., E. coli Marionette as described in the preceding Examples).

The fraction of enzymes found to be functional in E. coli under common conditions was surprisingly high, especially considering the diversity in the source genera and chemistries. The success rate was much higher than the successful transfer of other natural products genes, such as non-ribosomal peptide synthases, which also produce peptidic products. These results imply that RiPP enzymes can be combined from different sources to create synthetic pathways from which all the enzymes can be functionally expressed. Indeed, several examples have been published demonstrating the artificial combination of RiPP enzymes from different source species and pathways to make products not observed in nature [30, 75, 76]. Knowing that roughly half of RiPP enzymes are functionally compatible with E. coli dramatically expands the potential peptide chemical space that can be explored through the artificial mixing-and-matching of these enzymes. Fully enabling this requires a better understanding of the rules for designing precursor peptides that can be acted on by multiple modifying enzymes, such as the rules provided herein and in the preceding Examples. Collectively, these tools for the mining and de novo design of RiPPs enable the exploration of the vast universe of modified peptides for novel antibiotics, intercellular communication channels, and signaling molecules that influence animal and plant physiology.

### Materials and Methods

Strains, plasmids, media, and chemicals. E. coli NEB 10-beta (C3019I, New England BioLabs, Ipswich, Mass., USA) was used for all routine cloning. E. coli BL21 (C2530H, New England BioLabs, Ipswich, Mass., USA) was used to characterize RSTs and linker variants in low-throughput (flask) cultures. E. coli NEB Express (C2523I, New England BioLabs, Ipswich, Mass., USA) was used to express all other experiments. All plasmids containing RST-fused purcursor peptide genes use a pSC101 origin variant (var 2) with ampicillin resistance[77]. All plasmids carrying modifying enzyme genes contain p15A origins of replication and kanamycin resistance. LB-Miller media (B244620, BD, Franklin Lakes, N.J., USA) or TB media (T0311, Teknova, Hollister, Calif., USA) supplemented with 0.4% glycerol (BDH1172-4LP, VWR, OH, USA) were used for peptide expression and modification. 2xYT liquid media (B244020, BD, Franklin Lakes, N.J., USA) and 2xYT+2% agar (B214010, BD, Franklin Lakes, N.J., USA) plates were used for routine cloning and strain maintenance. SOB liquid media (S0210, Teknova, Hollister, Calif., USA) was used for making competent cells. SOC liquid media (B9020S, New England BioLabs, Iwsich, Mass., USA) was used for outgrowth. Cells were induced with the following chemicals: cumate (cuminic acid) ≥98% purity from Millipore Sigma (268402, Millipore Sigma, Saint Louis, Mo., USA) added as 1000× stock (200 mM) in EtOH or DMSO; isopropyl β-D-1-thiogalactopyranoside (IPTG) ≥99% purity (I2481C, Gold Biotechnology, Saint Louis, Mo., USA) added as 1000× stock (1 M) in water or DMSO; 3OC6-AHL from Millipore Sigma (K3007, Millipore Sigma, Saint Louis, Mo., USA) added as a 1000× stock (10 mM) in DMF. Cells were selected with the following antibiotics: 50 μg/ml kanamycin (K-120-10, Gold Biotechnology, Saint Louis, Mo., USA); 100 μg/ml carbenicillin (C-103-5, Gold Biotechnology, Saint Louis, Mo., USA); 30 μg/ml chloramphenicol. Liquid chromatography was performed with Optima Acetonitrile (A996-4, Thermo Fisher Scientific, MA, USA) and water (Milli-Q Advantage A10, Millipore Sigma, Saint Louis, Mo., USA) supplemented with LC-MS Grade Formic Acid (85178, Thermo Fisher Scientific). DNA oligos and gblocks were ordered from Integrated DNA Technologies (San Francisco, Calif., USA).

Gene design. A list of plasmids and corresponding plasmid maps are provided in Table 16. Amino acid sequences of all modifying enzymes and peptides are provided in Table 17. Sequences of genetic parts and full plasmids are provided in Table 18 and Table 19.

TABLE 16
Plasmids used in this Example

| Name | Origin | Marker | Backbone | Gene | Description |
|---|---|---|---|---|---|
| pEG1128 | p15A | Kan | bEG_S7 | truD | pLux modifying enzyme expression plasmid |
| pEG2192 | pSC101 var2 | Amp | bEG_S5 | papoA | RST_{N} peptide expression plasmid |
| pEG2194 | pSC101 var2 | Amp | bEG_S5 | bamA | RST_{N} peptide expression plasmid |
| pEG2195 | pSC101 var2 | Amp | bEG_S5 | epiA | RST_{N} peptide expression plasmid |
| pEG2199 | pSC101 var2 | Amp | bEG_S5 | halA1 | RST_{N} peptide expression plasmid |
| pEG2200 | pSC101 var2 | Amp | bEG_S5 | halA2 | RST_{N} peptide expression plasmid |
| pEG2312 | pSC101 var2 | Amp | bEG_S5 | papA_tev | RST_{N} peptide expression plasmid |
| pEG2575 | pSC101 var2 | Amp | bEG_S5 | psnA2_tev | RST_{N} peptide expression plasmid |
| pEG3017 | pSC101 var2 | Cm | bEG_S1 | truE* | MBP-tag peptide expression plasmid |
| pEG3045 | pSC101 var2 | Amp | bEG_S2 | mdnA | HIS-tag peptide expression plasmid |
| pEG3046 | pSC101 var2 | Amp | bEG_S2 | bmbC | HIS-tag peptide expression plasmid |
| pEG3047 | pSC101 var2 | Amp | bEG_S2 | strA | HIS-tag peptide expression plasmid |
| pEG3048 | pSC101 var2 | Amp | bEG_S2 | pqqA | HIS-tag peptide expression plasmid |
| pEG3049 | pSC101 var2 | Amp | bEG_S2 | sboA | HIS-tag peptide expression plasmid |
| pEG3051 | pSC101 var2 | Amp | bEG_S2 | tfxA | HIS-tag peptide expression plasmid |
| pEG3052 | pSC101 var2 | Amp | bEG_S2 | procA1.7 | HIS-tag peptide expression plasmid |
| pEG3053 | pSC101 var2 | Amp | bEG_S2 | tbtA | HIS-tag peptide expression plasmid |
| pEG3055 | pSC101 var2 | Amp | bEG_S2 | pgm2 | HIS-tag peptide expression plasmid |
| pEG3057 | pSC101 var2 | Amp | bEG_S3 | truE* | RST_{N}* peptide expression plasmid |
| pEG3058 | pSC101 var2 | Amp | bEG_S2 | mdnA | RST_{N}* peptide expression plasmid |
| pEG3059 | pSC101 var2 | Amp | bEG_S2 | sboA | RST_{N}* peptide expression plasmid |
| pEG3060 | pSC101 var2 | Amp | bEG_S2 | pqqA | RST_{N}* peptide expression plasmid |
| pEG3061 | pSC101 var2 | Amp | bEG_S2 | strA | RST_{N}* peptide expression plasmid |
| pEG3062 | pSC101 var2 | Amp | bEG_S2 | bmbC | RST_{N}* peptide expression plasmid |
| pEG3063 | pSC101 var2 | Amp | bEG_S2 | tfxA | RST_{N}* peptide expression plasmid |
| pEG3064 | pSC101 var2 | Amp | bEG_S2 | procA1.7 | RST_{N}* peptide expression plasmid |
| pEG3065 | pSC101 var2 | Amp | bEG_S2 | tbtA | RST_{N}* peptide expression plasmid |
| pEG3067 | pSC101 var2 | Amp | bEG_S2 | pgm2 | RST_{N}* peptide expression plasmid |
| pEG3121 | pSC101 var2 | Amp | bEG_S4 | mdnA* | RST_{N} peptide expression plasmid |
| pEG3128 | pSC101 var2 | Amp | bEG_S4 | procA* | RST_{N} peptide expression plasmid |
| pEG3132 | pSC101 var2 | Amp | bEG_S4 | paaP | RST_{N} peptide expression plasmid |
| pEG3157 | pSC101 var2 | Amp | bEG_S5 | mibA | RST_{N} peptide expression plasmid |
| pEG3161 | pSC101 var2 | Amp | bEG_S5 | plpA1 | RST_{N} peptide expression plasmid |
| pEG3162 | pSC101 var2 | Amp | bEG_S5 | plpA2 | RST_{N} peptide expression plasmid |
| pEG3165 | pSC101 var2 | Amp | bEG_S5 | pbtA | RST_{N} peptide expression plasmid |
| pEG3172 | pSC101 var2 | Amp | bEG_S5 | ltnA1 | RST_{N} peptide expression plasmid |
| pEG3173 | pSC101 var2 | Amp | bEG_S5 | ltnA2 | RST_{N} peptide expression plasmid |
| pEG3174 | pSC101 var2 | Amp | bEG_S5 | crnA1 | RST_{N} peptide expression plasmid |
| pEG3175 | pSC101 var2 | Amp | bEG_S5 | crnA2 | RST_{N} peptide expression plasmid |
| pEG3176 | pSC101 var2 | Amp | bEG_S5 | bsjA2 | RST_{N} peptide expression plasmid |
| pEG3177 | pSC101 var2 | Amp | bEG_S5 | bsjA3 | RST_{N} peptide expression plasmid |
| pEG3178 | pSC101 var2 | Amp | bEG_S5 | cinA | RST_{N} peptide expression plasmid |
| pEG3180 | pSC101 var2 | Amp | bEG_S5 | lasA | RST_{N} peptide expression plasmid |
| pEG3181 | pSC101 var2 | Amp | bEG_S5 | albsA | RST_{N} peptide expression plasmid |
| pEG3182 | pSC101 var2 | Amp | bEG_S5 | mcbA | RST_{N} peptide expression plasmid |
| pEG3194 | pSC101 var2 | Amp | bEG_S5 | psnA2 | RST_{N} peptide expression plasmid |
| pEG3197 | pSC101 var2 | Amp | bEG_S5 | aMdnA | RST_{N} peptide expression plasmid |
| pEG3212 | pSC101 var2 | Amp | bEG_S6 | capA | RST_{C} peptide expression plasmid |
| pEG3213 | pSC101 var2 | Amp | bEG_S6 | lasA | RST_{C} peptide expression plasmid |
| pEG3214 | pSC101 var2 | Amp | bEG_S6 | albsA | RST_{C} peptide expression plasmid |
| pEG3215 | pSC101 var2 | Amp | bEG_S6 | atxA1 | RST_{C} peptide expression plasmid |
| pEG3248 | pSC101 var2 | Amp | bEG_S4 | sboA | RST_{N} peptide expression plasmid |
| pEG3283 | pSC101 var2 | Amp | bEG_S5 | papA | RST_{N} peptide expression plasmid |
| pEG3286 | pSC101 var2 | Amp | bEG_S5 | pcpA | RST_{N} peptide expression plasmid |
| pEG3553 | pSC101 var2 | Amp | bEG_S6 | cln1A1 | RST_{C} peptide expression plasmid |
| pEG3554 | pSC101 var2 | Amp | bEG_S6 | cln1A2 | RST_{C} peptide expression plasmid |
| pEG3555 | pSC101 var2 | Amp | bEG_S6 | cln2A1 | RST_{C} peptide expression plasmid |
| pEG3556 | pSC101 var2 | Amp | bEG_S6 | cln2A2 | RST_{C} peptide expression plasmid |
| pEG3557 | pSC101 var2 | Amp | bEG_S6 | cln3A1 | RST_{C} peptide expression plasmid |
| pEG3558 | pSC101 var2 | Amp | bEG_S6 | cln3A2 | RST_{C} peptide expression plasmid |
| pEG3559 | pSC101 var2 | Amp | bEG_S6 | cln3A3 | RST_{C} peptide expression plasmid |
| pEG3560 | pSC101 var2 | Amp | bEG_S6 | csegA1 | RST_{C} peptide expression plasmid |
| pEG3561 | pSC101 var2 | Amp | bEG_S6 | csegA2 | RST_{C} peptide expression plasmid |
| pEG3562 | pSC101 var2 | Amp | bEG_S6 | csegA3 | RST_{C} peptide expression plasmid |
| pEG3563 | pSC101 var2 | Amp | bEG_S5 | padeA | RST_{N} peptide expression plasmid |
| pEG3564 | pSC101 var2 | Amp | bEG_S5 | thcoA | RST_{N} peptide expression plasmid |
| pEG3565 | pSC101 var2 | Amp | bEG_S5 | stspA | RST_{N} peptide expression plasmid |
| pEG3567 | pSC101 var2 | Amp | bEG_S5 | lcnA | RST_{N} peptide expression plasmid |
| pEG3568 | pSC101 var2 | Amp | bEG_S5 | pal A | RST_{N} peptide expression plasmid |
| pEG3570 | pSC101 var2 | Amp | bEG_S5 | raxX | RST_{N} peptide expression plasmid |
| pEG3571 | pSC101 var2 | Amp | bEG_S5 | comX | RST_{N} peptide expression plasmid |
| pEG3572 | pSC101 var2 | Amp | bEG_S5 | kgpE | RST_{N} peptide expression plasmid |
| pEG3574 | pSC101 var2 | Amp | bEG_S5 | tgnA* | RST_{N} peptide expression plasmid |
| pEG3871 | pSC101 var2 | Amp | bEG_S5 | sgbA | RST_{N} peptide expression plasmid |
| pEG3905 | pSC101 var2 | Amp | bEG_S5 | truE | RST_{N} peptide expression plasmid |
| pEG7034 | p15A | Kan | bEG_S9 | truD | pCym modifying enzyme expression plasmid |
| pEG7035 | p15A | Kan | bEG_S9 | alba | pCym modifying enzyme expression plasmid |
| pEG7037 | p15A | Kan | bEG_S9 | mdnC | pCym modifying enzyme expression plasmid |
| pEG7043 | p15A | Kan | bEG_S9 | procM | pCym modifying enzyme expression plasmid |
| pEG7047 | p15A | Kan | bEG_S9 | mibHS | pCym modifying enzyme expression plasmid |
| pEG7048 | p15A | Kan | bEG_S9 | mibD | pCym modifying enzyme expression plasmid |
| pEG7056 | p15A | Kan | bEG_S9 | plpXY | pCym modifying enzyme expression plasmid |
| pEG7058 | p15A | Kan | bEG_S9 | pbtO | pCym modifying enzyme expression plasmid |
| pEG7059 | p15A | Kan | bEG_S9 | pbtM1 | pCym modifying enzyme expression plasmid |
| pEG7060 | p15A | Kan | bEG_S9 | paaA | pCym modifying enzyme expression plasmid |
| pEG7066 | p15A | Kan | bEG_S9 | cinX | pCym modifying enzyme expression plasmid |
| pEG7067 | p15A | Kan | bEG_S9 | capBC | pCym modifying enzyme expression plasmid |
| pEG7068 | p15A | Kan | bEG_S9 | lasBCD | pCym modifying enzyme expression plasmid |
| pEG7069 | p15A | Kan | bEG_S9 | lasF | pCym modifying enzyme expression plasmid |
| pEG7070 | p15A | Kan | bEG_S9 | albsBC | pCym modifying enzyme expression plasmid |
| pEG7071 | p15A | Kan | bEG_S9 | albsT | pCym modifying enzyme expression plasmid |
| pEG7073 | p15A | Kan | bEG_S9 | mcbCD | pCym modifying enzyme expression plasmid |
| pEG7074 | p15A | Kan | bEG_S9 | mibO | pCym modifying enzyme expression plasmid |
| pEG7076 | p15A | Kan | bEG_S9 | ltnM1 | pCym modifying enzyme expression plasmid |
| pEG7077 | p15A | Kan | bEG_S9 | ltnM2 | pCym modifying enzyme expression plasmid |
| pEG7078 | p15A | Kan | bEG_S9 | crnM | pCym modifying enzyme expression plasmid |
| pEG7079 | p15A | Kan | bEG_S9 | bsjM | pCym modifying enzyme expression plasmid |
| pEG7127 | p15A | Kan | bEG_S9 | psnB | pCym modifying enzyme expression plasmid |
| pEG7130 | p15A | Kan | bEG_S9 | amdnC | pCym modifying enzyme expression plasmid |
| pEG7132 | p15A | Kan | bEG_S9 | atxBC | pCym modifying enzyme expression plasmid |
| pEG7133 | p15A | Kan | bEG_S9 | cln1BC | pCym modifying enzyme expression plasmid |
| pEG7134 | p15A | Kan | bEG_S9 | cln2BC | pCym modifying enzyme expression plasmid |
| pEG7135 | p15A | Kan | bEG_S9 | cln3BC | pCym modifying enzyme expression plasmid |
| pEG7136 | p15A | Kan | bEG_S9 | csegBC | pCym modifying enzyme expression plasmid |
| pEG7137 | p15A | Kan | bEG_S9 | padeK | pCym modifying enzyme expression plasmid |
| pEG7138 | p15A | Kan | bEG_S9 | thcoK | pCym modifying enzyme expression plasmid |
| pEG7139 | p15A | Kan | bEG_S9 | stspM | pCym modifying enzyme expression plasmid |
| pEG7141 | p15A | Kan | bEG_S9 | lcnG | pCym modifying enzyme expression plasmid |
| pEG7142 | p15A | Kan | bEG_S9 | palS | pCym modifying enzyme expression plasmid |
| pEG7143 | p15A | Kan | bEG_S9 | sgbL | pCym modifying enzyme expression plasmid |
| pEG7144 | p15A | Kan | bEG_S9 | raxST | pCym modifying enzyme expression plasmid |
| pEG7145 | p15A | Kan | bEG_S9 | comQ | pCym modifying enzyme expression plasmid |
| pEG7146 | p15A | Kan | bEG_S9 | kgpF | pCym modifying enzyme expression plasmid |
| pEG7147 | p15A | Kan | bEG_S9 | tgnB | pCym modifying enzyme expression plasmid |
| pEG7149 | p15A | Kan | bEG_S9 | papB | pCym modifying enzyme expression plasmid |
| pEG7152 | p15A | Kan | bEG_S9 | pcpXY | pCym modifying enzyme expression plasmid |
| pEG7160 | p15A | Kan | bEG_S9 | lynD | pCym modifying enzyme expression plasmid |
| pEG7166 | p15A | Kan | bEG_S9 | papoK | pCym modifying enzyme expression plasmid |
| pEG7169 | p15A | Kan | bEG_S9 | epiD | pCym modifying enzyme expression plasmid |
| pEG7171 | p15A | Kan | bEG_S9 | bamB | pCym modifying enzyme expression plasmid |
| pEG7172 | p15A | Kan | bEG_S8 | halM1 | pCym modifying enzyme expression plasmid |
| pEG7173 | p15A | Kan | bEG_S8 | halM2 | pCym modifying enzyme expression plasmid |

Peptide expression/modification from flasks and purification. Plasmids were transformed into E. coli BL21, struck out on 2xYT agar with carbenicillin (or chloramphenicol for pEG3017) and kanamycin (if co-transforming modifying enzyme) and incubated (30° C., overnight). Individual colonies were used to inoculate 3 mL of LB media in a culture tube (352059, Corning, N.Y., USA) and incubated overnight (30° C., 250 r.p.m.) in an Innova44 (Eppendorf, N.Y., USA). Aliquots (500 l) were taken from the overnight cultures and subcultured into 50 mL of LB media in a 250 mL Erlenmeyer flask. After 3 hours incubation (Innova44, 30° C., 250 r.p.m.), IPTG and 3OC6-AHL (if inducing modifying enzyme) was added to final concentrations of 1 mM and 10 μM and cultures were incubated for 20 hours (Innova44, 18° C., 250 r.p.m.) (note: IPTG was not added for pEG3017, where the MBP-tagged peptide is constitutively expressed). The 50 mL cultures were transferred to a falcon tube (352070, Corning, N.Y., USA), centrifuged (4,500 g, 4° C., 20 min) in a Sorvall Legend XFR Centrifuge (Thermo Fisher Scientific, MA, USA), pellets were resuspended in 600 μl lysis buffer (5 M guanidinium hydrochloride, 300 mM NaCl, 50 mM sodium phosphate, pH 7.5), and freeze-thawed twice (frozen in −80° C. freezer; thawed in innova44 incubator at 30° C., 250 r.p.m). Cell lysates were centrifuged (Eppendorf 5424, 21,130 g, room temperature, 15 min) in an Eppendorf 5424 Centrifuge (Eppendorf, N.Y., USA) and the peptides affinity purified using His SpinTrap TALON columns (29-0005-93, GE Life Sciences (now Cytiva), Marlborough, Mass., USA), following manufacturer instructions, using 600 μL lysis buffer twice for column equilibration, loading 600 □L clarified lysate, two washes with 600 μL wash buffer (300 mM NaCl, 50 mM sodium phosphate, 5 mM imidazole, pH 7.5), and 200 μL elution buffer (300 mM NaCl, 50 mM sodium phosphate, 200 mM imidazole, pH 7.5) for elution. Purifications used an Eppendorf 5424 centrifuge.

Calculation of peptide molar masses. For large peptides/proteins, mass was calculated as described for ESIprot79: five consecutively charged m/z's (m1, m2, m3, m4, m5) were taken from the spectra and used to calculate the charge states (z1, z2, z3, z4, z5) for each of the peaks. For peaks m1 and m2, which have charge states, z1 and z2, where z2=z1−1 (peak 1 has one proton more than peak 2): z1=(m2−1)/(m2−m1). Charges z1, z2, z3, and z4 were calculated using each of the four pairs of consecutively charged masses (m1 and m2, m2 and m3, m3 and m4, m4 and m5), subtracted by the number of protons the peak has compared to m5, and averaged together and rounded to the nearest integer to calculate the lowest charge (z5). Charges z1-4 are recalculated based on charge z5 (z1=z5+4, z2=z5+3, etc.), uncharged masses are calculated from each of the five m/z's: uncharged mass=zx(observed m/z)−zx.

Peptide expression in 96-well plates. Plasmids were transformed into E. coli NEB Express using 15 μL of competent cells and 1 μL of each plasmid being transformed in a 96-well PCR plate (1402-9596, USA Scientific, FL, USA or 951020401, Eppendorf, N.Y., USA). Transformations were incubated on ice (20-30 min), heat shocked (40° C., 30 sec), and incubated on ice again (5 min). Cells were then transferred to a deep well 96-well plate (1896-2000, USA Scientific, FL, USA) with 100 μL of SOC media. After outgrowth (Multitron Pro, 1 hr, 37° C.) in an Infors HT Multitron Pro (Infors USA, MD, USA), 400 μL LB media was added with appropriate antibiotics (100 μg/ml carbenicillin and 50 μg/ml kanamycin) and incubated (Multitron Pro, 30° C., 900 r.p.m.) until all wells reached stationary phase (cultures were visibly saturated, 12-30 hours). Overnight cultures were diluted 1:100 into 1 mL LB or TB media (with same antibiotics as previous culture) in deep well plates. After a 3 hour incubation (Multitron Pro, 30° C., 900 r.p.m.), appropriate inducer was added (1 mM IPTG or 200 μM cumate) and cultures were incubated for 20 hours (Multitron Pro, 30° C., 900 r.p.m.). The 96-well plates were centrifuged (Legend XFR, 4,500 g, 4° C., 20 min) and media discarded. Pellets were either purified immediately or frozen at −20 C until purification.

Haloduracin production and purification. Haloduracin was produced following the 96-well expression protocol described above, with each sample being produced in two wells of 1 mL TB media to double the amount of product produced. Culture pellets were resuspended in 800 L lysis buffer, freeze-thawed (frozen at −80° C.; thawed in Multitron Pro at 37° C., 900 r.p.m), and centrifuged (Legend XFR, 4,500 g, 4° C., 30 min). Peptides were affinity purified using HIS MultiTrap TALON plates, using 500 μL water and two 500 μL lysis buffer washes for column equilibration (Legend XFR, 500 g, 4° C., 2 min), loading 600 μL of both matching sample's clarified lysates iteratively (load one, then centrifuge, then load the second, then centrifuge) (Legend XFR, 100 g, 4° C., 5 min), washing twice with 500 μL wash buffer, and eluting three times with 200 μL elution buffer to maximize titer. Purification was followed by solid-phase extraction (SPE) using Strata-XL microtiter plates (8E-S043-TGB, Phenomenex, CA, USA). Plates were conditioned with 1 mL methanol wash followed by 1 mL water wash. All 600 μL of TALON eluent was loaded, washed twice with 1 mL water, and then eluted twice with 500 μl 1:1 acetonitrile:water (supplemented with 0.1% formic acid). Plates with eluent were dried down at room temperature in a Savant Speedvac SPD2010 (Thermo Fisher Scientific, MA, USA), samples resuspended in 40 μL TE buffer (10 mM tris, 1 mM EDTA) with 20 μL 2 mg/mL TEV protease, and then incubated (stationary, 30° C., 8 hr). Cut fractions were desalted using a Strata-X SPE plate (8E-S100-TGB, Phenomenex, CA, USA) with same condition/wash/elution/drying steps as above. Dried down samples were resuspended in 50 μL 1:1 methanol:water.

Proteolytic cleavage and removal of SUMO. For purification of haloduracin for antimicrobial assays, TEV protease was purified as described previously 78 [Addgene #8827, concentrated to 2 mg/mL in TEV buffer (25 mM Tris-HCl, pH 8.0, 50 mM NaCl, 1 mM TCEP, 50% glycerol)]. For MS/MS analysis, TEV protease was prepared as a 50 mg/mL solution of 10% (w/w) TEV lyophilizate (Gene and Cell Technologies, CA, USA) in TEV Buffer.

### REFERENCES FROM EXAMPLE 8

1. Zhang, M. M.; et al., Expert Opinion on Drug Discovery 2017, 12 (5), 475-487.
2. Smanski, M. J.; et al., Nature Reviews Microbiology 2016, 14 (3), 135-149.
3. Medema, M. H.; Fischbach, M. A., Nature Chemical Biology 2015, 11 (9), 639-648.
4. Bayer, T. S.; et al., Journal of the American Chemical Society 2009, 131 (18), 6508-6515.
5. Freeman, M. F.; et al., Science 2012, 338 (6105), 387-390.
6. Guo, C.-J.; et al., Cell 2017, 168 (3), 517-526.e18.
7. Arnison, P. G.; et al., Nat. Prod. Rep. 2013, 30 (1), 108-160.
8. Lin, P. F.; et al., Antimicrobial Agents and Chemotherapy 1996, 40 (1), 133-138.
9. Jin, M.; et al., Angewandte Chemie International Edition 2003, 42 (25), 2898-2901.
10. Potterat, O.; et al., Journal of Natural Products 2004, 67 (9), 1528-1531.
11. Yano, K.; et al., The Journal of Antibiotics 1995, 48 (11), 1368-1370.
12. Vogt, E.; Künzler, M., Applied Microbiology and Biotechnology 2019, 103 (14), 5567-5581.
13. Skinnider, M. A.; et al., Proceedings of the National Academy of Sciences 2016, 113 (42), E6343-E6351.
14. Montalbán-López, M.; et al., Natural Product Reports 2021.
15. Li, Y., Protein Expression and Purification 2011, 80 (2), 260-267.
16. Shi, Y.; et al., Journal of the American Chemical Society 2011, 133 (8), 2338-2341.
17. Donia, M. S.; et al., Nature Chemical Biology 2008, 4 (6), 341-343.
18. Zhang, Y., et al., Nature Communications 2018, 9 (1).
19. Lee, H., et al., Biochemistry 2017, 56 (37), 4927-4930.
20. Meulenberg, J., FEMS Microbiology Letters 1990, 71 (3), 337-343.
21. Morinaka, B. I.; et al., Science 2018, 359 (6377), 779-782.
22. Himes, P. M.; et al., ACS Chemical Biology 2016, 11 (6), 1737-1744.
23. Zhang, Z.; et al., Journal of the American Chemical Society 2016, 138 (48), 15511-15514.
24. Van Staden, A. D. P et al., ACS Synthetic Biology 2019, 8 (10), 2220-2227.
25. Zhu, S.; et al., J Biol Chem 2016, 291 (26), 13662-78.
26. Hegemann, J. D.; et al., Journal of the American Chemical Society 2013, 135 (1), 210-222.
27. Ren, H.; et al., ACS Chemical Biology 2018, 13 (10), 2966-2972.
28. Serebryakova, M.; et al., Journal of the American Chemical Society 2016, 138 (48), 15690-15698.
29. Weiz, R., et al., Chemistry & Biology 2011, 18 (11), 1413-1421.
30. Burkhart, B. J.; et al., ACS Central Science 2017, 3 (6), 629-638.
31. Bhushan, A.; et al., Nature Chemistry 2019, 11 (10), 931-939.
32. Young, S., et al., Chemistry & Biology 2012, 19 (12), 1600-1610.
33. Knappe, T. A.; et al., Journal of the American Chemical Society 2008, 130 (34), 11446-11454.
34. Mcclerren, A. L.; et al., Proceedings of the National Academy of Sciences 2006, 103 (46), 17243-17248.
35. Hudson, G. A.; et al., Journal of the American Chemical Society 2015, 137 (51), 16012-16015.
36. Reyna-Gonzilez, E.; et al., Angewandte Chemie International Edition 2016, 55 (32), 9398-9401.
37. Vinogradov, A. A.; et al., Nature Communications 2020, 11 (1).
38. Rosano, G. N. L.; et al., Frontiers in Microbiology 2014, 5.
39. Panavas, T.; et al., Methods in Molecular Biology, Humana Press: 2009; pp 303-317.
40. Gaglione, R.; et al., New Biotechnology 2019, 51, 39-48.
41. Satakarni, M.; et al., Protein Expression and Purification 2011, 78 (2), 113-119.
42. He, J.; et al., Molecules 2018, 23 (9), 2246.
43. Ma, Q.; et al., The Journal of Microbiology 2012, 50 (2), 326-331.
44. Nguyen, T. Q. N.; et al., Nature Chemistry 2020, 12 (11), 1042-1053.
45. Meyer, A. J.; et al., Nature Chemical Biology 2019, 15 (2), 196-204.
46. Rocco, C. J.; et al., Plasmid 2008, 59 (3), 231-237.
47. Mrksich, M., ACS Nano 2008, 2 (1), 7-18.
48. Huang, C.-F.; et al., ACS Combinatorial Science 2019, 21 (11), 760-769.
49. Espah Borujeni, A.; et al., Nucleic Acids Research 2014, 42 (4), 2646-2659.
50. Salis, H. M.; et al., Nature Biotechnology 2009, 27 (10), 946-950.
51. Hou, Y.; et al., Organic Letters 2012, 14 (19), 5050-5053.
52. Schramma, K. R.; et al., Nature Chemistry 2015, 7 (5), 431-437.
53. Babasaki, K.; et al., The Journal of Biochemistry 1985, 98 (3), 585-603.
54. Breil, B.; et al., Journal of bacteriology 1996, 178 (14), 4150-4156.
55. Li, B.; et al., Proceedings of the National Academy of Sciences 2010, 107 (23), 10430-10435.
56. Morris, R. P.; et al., Journal of the American Chemical Society 2009, 131 (16), 5946-5955.
57. Noike, M.; et al., Nature Chemical Biology 2015, 11 (1), 71-76.
58. Cooper, L. E.; et al., Chemistry & Biology 2008, 15 (10), 1035-1045.
59. Zhang, Q.; et al., Proceedings of the National Academy of Sciences 2014, 111 (33), 12031-12036.
60. Caruso, A.; et al., Journal of the American Chemical Society 2019, 141 (42), 16610-16614.
61. Hudson, G. A.; et al., Journal of the American Chemical Society 2019.
62. Shuguo, H.; et al., Applied Biochemistry and Biotechnology 2012, 166 (5), 1368-1379.
63. Zimmermann, M.; et al., Chem. Sci. 2014, 5 (10), 4032-4043.
64. Zhu, S.; et al., FEBS Letters 2016, 590 (19), 3323-3334.
65. Gavrish, E.; et al., Chemistry & Biology 2014, 21 (4), 509-518.
66. Ghodge, S. V.; et al., Journal of the American Chemical Society 2016, 138 (17), 5487-5490.
67. Luu, D. D.; et al., Proceedings of the National Academy of Sciences 2019, 116 (17), 8525-8534.
68. Kupke, T.; et al., Journal of Biological Chemistry 1995, 270 (19), 11282-11289.
69. Sardar, D.; et al., ACS Synthetic Biology 2015, 4 (2), 167-176.
70. Casini, A.; et al., Journal of the American Chemical Society 2018, 140 (12), 4302-4316.
71. Hillson, N.; et al., Nature Communications 2019, 10 (1).
72. Voigt, C. A., Nature Communications 2020, 11 (1).
73. Kloosterman, A. M.; et al., mSystems 2020, 5 (5).
74. Tietz, J. I.; et al., Nature Chemical Biology 2017, 13 (5), 470-478.
75. Sardar, D.; et al., Chemistry & Biology 2015, 22 (7), 907-916.
76. Van Heel, A. J.; et al., ACS Synthetic Biology 2013, 2 (7), 397-404.
77. Segall-Shapiro, T. H.; et al., Nature Biotechnology 2018, 36 (4), 352-358.
78. Tropea, J. E.; et al., Methods in Molecular Biology, Humana Press: 2009; pp 297-307.
79. Winkler, R., Rapid Communications in Mass Spectrometry 2010, 24 (3), 285-294.
80. Patiny, L.; et al., Journal of Chemical Information and Modeling 2013, 53 (5), 1223-1228.

### SEQUENCES USED IN THE EXAMPLES

TABLE 3
Non-limiting example of peptides (e.g., modified peptides)

| | Peptide | Leader | Core | | |
|---|---|---|---|---|---|
| Name | Sequence | sequence | sequence | Mod | Coding sequence (CDS) |
| gAMK- | MLKQINVIAGV | MLKQINVIAGV | ACTACEQCSK | L5 | ATGCTGAAACAGATCAAC |
| 174 | KEPIRAYACTA | KEPIRAY | CDTNEK | | GTTATTGCGGGTGTGAAA |
| | CEQCSKCDTNE | (SEQ ID NO: 46) | (SEQ ID NO: 6) | | GAGCCGATTCGCGCGTAC |
| | K | | | | GCCTGTACCGCATGTGAG |
| | (SEQ ID NO: 26) | | | | CAATGCAGTAAATGTGAC |
| | | | | | ACCAATGAGAAG |
| | | | | | (SEQ ID NO: 47) |
| gAMK- | MLKQINVIAGV | MLKQINVIAGV | ECPADETCMH | L5 | ATGCTGAAACAGATAAAC |
| 175 | KEPIRAYECPA | KEPIRAY | CESHEM | | GTCATTGCAGGCGTCAAG |
| | DETCMHCESH | (SEQ ID NO: 46) | (SEQ ID NO: 7) | | GAACCCATTCGCGCGTAT |
| | EM | | | | GAATGTCCGGCCGATGAA |
| | (SEQ ID NO: 27) | | | | ACTTGTATGCATTGCGAAT |
| | | | | | CGCATGAGATG |
| | | | | | (SEQ ID NO: 48) |
| gAMK- | MLKQINVIAGV | MLKQINVIAGV | HCIFIESCDVC | L5 | ATGCTGAAACAGATCAAC |
| 176 | KEPIRAYHCIFI | KEPIRAY | ELNEP | | GTGATAGCCGGGGTCAAA |
| | ESCDVCELNEP | (SEQ ID NO: 46) | (SEQ ID NO: 8) | | GAGCCCATTCGCGCATAT |
| | (SEQ ID NO: 28) | | | | CACTGCATTTTTATTGAAA |
| | | | | | GCTGTGACGTGTGCGAAC |
| | | | | | TGAATGAACCG |
| | | | | | (SEQ ID NO: 49) |
| gAMK- | MLKQINVIAGV | MLKQINVIAGV | KCEKREECAD | L5 | ATGCTGAAGCAAATCAAC |
| 177 | KEPIRAYKCEK | KEPIRAY | CDHLEF | | GTTATCGCCGGAGTTAAG |
| | REECADCDHLE | (SEQ ID NO: 46) | (SEQ ID NO: 9) | | GAACCTATTCGTGCGTATA |
| | F | | | | AATGTGAAAAACGGGAAG |
| | (SEQ ID NO: 29) | | | | AGTGTGCTGATTGCGATC |
| | | | | | ACCTTGAATTT |
| | | | | | (SEQ ID NO: 50) |
| gAMK- | MLKQINVIAGV | MLKQINVIAGV | KCTSKECCIQC | L5 | ATGCTGAAACAGATCAAC |
| 178 | KEPIRAYKCTS | KEPIRAY | EGSES | | GTCATTGCCGGCGTCAAA |
| | KECCIQCEGSE | (SEQ ID NO: 46) | (SEQ ID NO: | | GAACCAATCCGTGCTTAC |
| | S | | 10) | | AAGTGTACGTCAAAAGAA |
| | (SEQ ID NO: 30) | | | | TGCTGTATCCAGTGTGAA |
| | | | | | GGAAGTGAAAGC |
| | | | | | (SEQ ID NO: 51) |
| gAMK- | MLKQINVIAGV | MLKQINVIAGV | MCVFCEICVM | L5 | ATGTTAAAACAAATTAAC |
| 179 | KEPIRAYMCVF | KEPIRAY | CDTHEM | | GTGATCGCCGGGGTTAAA |
| | CEICVMCDTHE | (SEQ ID NO: 46) | (SEQ ID NO: | | GAACCCATCCGTGCGTAT |
| | M | | 11) | | ATGTGTGTATTTTGTGAAA |
| | (SEQ ID NO: 31) | | | | TTTGTGTGATGTGTGACAC |
| | | | | | CCATGAAATG |
| | | | | | (SEQ ID NO: 52) |
| gAMK- | MLKQINVIAGV | MLKQINVIAGV | PCGKREPCNT | L5 | ATGCTGAAGCAGATAAAT |
| 180 | KEPIRAYPCGK | KEPIRAY | CEHFET | | GTTATCGCGGGCGTCAAG |
| | REPCNTCEHFE | (SEQ ID NO: 46) | (SEQ ID NO: | | GAACCGATCCGTGCCTAT |
| | T | | 12) | | CCGTGTGGTAAACGCGAG |
| | (SEQ ID NO: 32) | | | | CCGTGTAATACCTGCGAA |
| | | | | | CATTTCGAAACG |
| | | | | | (SEQ ID NO: 53) |
| gAMK- | MLKQINVIAGV | MLKQINVIAGV | PCTTTEACTA | L5 | ATGCTGAAACAGATCAAC |
| 181 | KEPIRAYPCTT | KEPIRAY | CDSSDA | | GTCATTGCTGGTGTTAAAG |
| | TEACTACDSSD | (SEQ ID NO: 46) | (SEQ ID NO: | | AACCGATTCGCGCTTATCC |
| | A | | 13) | | GTGTACCACCACGGAAGC |
| | (SEQ ID NO: 33) | | | | GTGCACAGCCTGCGATTCT |
| | | | | | AGTGATGCG |
| | | | | | (SEQ ID NO: 54) |
| gAMK- | MLKQINVIAGV | MLKQINVIAGV | RCRCPENCLS | L5 | ATGCTGAAACAGATTAAC |
| 182 | KEPIRAYRCRC | KEPIRAY | CEPPER | | GTTATCGCGGGCGTCAAA |
| | PENCLSCEPPE | (SEQ ID NO: 46) | (SEQ ID NO: | | GAACCCATCAGAGCGTAT |
| | R | | 14) | | CGTTGTCGTTGCCCTGAGA |
| | (SEQ ID NO: 34) | | | | ACTGCCTGTCGTGCGAAC |
| | | | | | CGCCGGAGCGT |
| | | | | | (SEQ ID NO: 55) |
| gAMK- | MLKQINVIAGV | MLKQINVIAGV | SCTPDEVCPLC | L5 | ATGCTGAAGCAAATCAAT |
| 183 | KEPIRAYSCTP | KEPIRAY | EPCEP | | GTGATCGCGGGCGTTAAA |
| | DEVCPLCEPCE | (SEQ ID NO: 46) | (SEQ ID NO: | | GAGCCGATCCGGGCCTAC |
| | P | | 15) | | TCTTGTACCCCGGATGAA |
| | (SEQ ID NO: 35) | | | | GTATGTCCGCTCTGCGAGC |
| | | | | | CATGCGAACCG |
| | | | | | (SEQ ID NO: 56) |
| gAMK- | MLKQINVIAGV | MLKQINVIAGV | TCTMAEKCQI | L5 | ATGCTGAAGCAAATTAAC |
| 184 | KEPIRAYTCTM | KEPIRAY | CDVSEG | | GTGATTGCTGGTGTCAAG |
| | AEKCQICDVSE | (SEQ ID NO: 46) | (SEQ ID NO: | | GAACCTATCCGTGCGTAC |
| | G | | 16) | | ACATGTACGATGGCGGAG |
| | (SEQ ID NO: 36) | | | | AAATGCCAAATTTGCGAT |
| | | | | | GTGAGCGAAGGG |
| | | | | | (SEQ ID NO: 57) |
| gAMK- | MLKQINVIAGV | MLKQINVIAGV | ACTNPDPCTD | L3 | ATGCTCAAACAAATCAAC |
| 185 | KAPIRAYACTN | KAPIRAY | EEI | | GTGATCGCGGGAGTCAAA |
| | PDPCTDEEI | (SEQ ID NO: 46) | (SEQ ID NO: | | GCACCGATCCGCGCCTAC |
| | (SEQ ID NO: 37) | | 17) | | GCTTGCACAAACCCGGAC |
| | | | | | CCTTGCACGGATGAAGAA |
| | | | | | ATC |
| | | | | | (SEQ ID NO: 58) |
| gAMK- | MLKQINVIAGV | MLKQINVIAGV | PCEVLDNCTN | L3 | ATGCTTAAGCAGATAAAC |
| 186 | KAPIRAYPCEV | KAPIRAY | PDH | | GTGATCGCCGGCGTGAAA |
| | LDNCTNPDH | (SEQ ID NO: 46) | (SEQ ID NO: | | GCGCCGATCCGCGCGTAC |
| | (SEQ ID NO: 38) | | 18) | | CCGTGTGAAGTGTTGGAT |
| | | | | | AATTGCACAAATCCAGAC |
| | | | | | CAT |
| | | | | | (SEQ ID NO: 59) |
| gAMK- | MLKQINVIAGV | MLKQINVIAGV | ACTNPDPCTD | L3 | ATGCTGAAGCAAATCAAT |
| 187 | KEPIRAYACTN | KEPIRAY | EEI | | GTGATTGCCGGGGTAAAA |
| | PDPCTDEEI | (SEQ ID NO: 46) | (SEQ ID NO: | | GAACCGATACGCGCGTAC |
| | (SEQ ID NO: 39) | | 19) | | GCCTGTACTAACCCTGATC |
| | | | | | CGTGTACCGATGAGGAAA |
| | | | | | TC |
| | | | | | (SEQ ID NO: 60) |
| gAMK- | MLKQINVIAGV | MLKQINVIAGV | KCDEGDHCGT | L3 | ATGCTGAAACAGATTAAT |
| 188 | KEPIRAYKCDE | KEPIRAY | KDL | | GTGATTGCCGGAGTTAAG |
| | GDHCGTKDL | (SEQ ID NO: 46) | (SEQ ID NO: | | GAACCAATTCGCGCTTAT |
| | (SEQ ID NO: 40) | | 20) | | AAATGCGACGAAGGTGAT |
| | | | | | CATTGTGGCACTAAAGAT |
| | | | | | CTG |
| | | | | | (SEQ ID NO: 61) |
| gAMK- | MLKQINVIAGV | MLKQINVIAGV | PCEVLDNCTK | L3 | ATGCTGAAACAGATTAAT |
| 189 | KEPIRAYPCEV | KEPIRAY | PDH | | GTGATCGCGGGTGTAAAG |
| | LDNCTKPDH | (SEQ ID NO: 46) | (SEQ ID NO: | | GAACCGATCAGAGCGTAT |
| | (SEQ ID NO: 41) | | 21) | | CCATGCGAAGTTTTAGAC |
| | | | | | AACTGCACTAAACCCGAC |
| | | | | | CAC |
| | | | | | (SEQ ID NO: 62) |
| gAMK- | MLKQINVIAGV | MLKQINVIAGV | PCEVLDNCTN | L3 | ATGCTGAAACAAATTAAC |
| 190 | KEPIRAYPCEV | KEPIRAY | PDH | | GTTATTGCGGGTGTTAAA |
| | LDNCTNPDH | (SEQ ID NO: 46) | (SEQ ID NO: | | GAACCGATCCGTGCCTAT |
| | (SEQ ID NO: 42) | | 22) | | CCATGCGAGGTGTTGGAT |
| | | | | | AATTGCACCAACCCTGAT |
| | | | | | CAT |
| | | | | | (SEQ ID NO: 63) |
| gAMK- | MLKQINVIAGV | MLKQINVIAGV | QCPWHERCD | L3 | ATGTTAAAGCAGATCAAT |
| 191 | KEPIRAYQCPW | KEPIRAY | QCEP | | GTGATCGCAGGGGTGAAA |
| | HERCDQCEP | (SEQ ID NO: 46) | (SEQ ID NO: | | GAACCGATACGCGCATAC |
| | (SEQ ID NO: 43) | | 23) | | CAGTGCCCATGGCATGAA |
| | | | | | CGTTGTGATCAGTGCGAG |
| | | | | | CCG |
| | | | | | (SEQ ID NO: 64) |
| gAMK- | MLKQINVIAGV | MLKQINVIAGV | VCKYGEWCEI | L3 | ATGCTGAAGCAGATTAAC |
| 192 | KEPIRAYVCKY | KEPIRAY | VEI | | GTTATTGCCGGAGTTAAA |
| | GEWCEIVEI | (SEQ ID NO: 46) | (SEQ ID NO: | | GAACCCATACGCGCGTAC |
| | (SEQ ID NO: 44) | | 24) | | GTGTGTAAATATGGTGAA |
| | | | | | TGGTGTGAGATCGTCGAA |
| | | | | | ATC |
| | | | | | (SEQ ID NO: 65) |
| gAMK- | MLKQINVIAGV | MLKQINVIAGV | YCNITERCHS | L3 | ATGCTTAAACAAATTAAC |
| 193 | KEPIRAYYCNI | KEPIRAY | DEH | | GTGATCGCTGGTGTTAAG |
| | TERCHSDEH | (SEQ ID NO: 46) | (SEQ ID NO: | | GAACCGATCCGCGCGTAT |
| | (SEQ ID NO: 45) | | 25) | | TATTGCAATATCACCGAA |
| | | | | | CGCTGCCATTCGGATGAG |
| | | | | | CAT |
| | | | | | (SEQ ID NO: 66) |

The protein modification enzyme used with the sequences in Table 3 was PapB. The modification (mod) refers to the scaffold for the core peptide and correspond to L3 and L5 in FIG. 3.

TABLE 4
Non-limiting examples of protein modification enzyme sequences

| Protein | |
|---|---|
| modification | |
| enzyme | Amino acid sequence |
| LynD | MQSTPLLQIQPHFHVEVIEPKQVYLLGEQANHALTGQLYCQILPLLNGQYTLEQIVE |
| | KLDGEVPPEYIDYVLERLAEKGYLTEAAPELSSEVAAFWSELGIAPPVAAEALRQPV |
| | TLTPVGNISEVTVAALTTALRDIGISVQTPTEAGSPTALNVVLTDDYLQPELAKINKQ |
| | ALESQQTWLLVKPVGSVLWLGPVFVPGKTGCWDCLAHRLRGNREVEASVLRQKQ |
| | AQQQRNGQSGSVIGCLPTARATLPSTLQTGLQFAATEIAKWIVKYHVNATAPGTVF |
| | FPTLDGKIITLNHSILDLKSHILIKRSQCPTCGDPKILQHRGFEPLKLESRPKQFTSDGG |
| | HRGTTPEQTVQKYQHLISPVTGVVTELVRITDPANPLVHTYRAGHSFGSATSLRGLR |
| | NTLKHKSSGKGKTDSQSKASGLCEAVERYSGIFQGDEPRKRATLAELGDLAIHPEQC |
| | LCFSDGQYANRETLNEQATVAHDWIPQRFDASQAIEWTPVWSLTEQTHKYLPTALC |
| | YYHYPLPPEHRFARGDSNGNAAGNTLEEAILQGFMELVERDGVALWWYNRLRRPA |
| | VDLGSFNEPYFVQLQQFYRENDRDLWVLDLTADLGIPAFAGVSNRKTGSSERLILGF |
| | GAHLDPTIAILRAVTEVNQIGLELDKVPDENLKSDATDWLITEKLADHPYLLPDTTQ |
| | PLKTAQDYPKRWSDDIYTDVMTCVNIAQQAGLETLVIDQTRPDIGLNVVKVTVPG |
| | MRHFWSRFGEGRLYDVPVKLGWLDEPLTEAQMNPTPMPF (SEQ ID NO: 80) |
| PapB | MANLIQDREDELIHFHPYKLFEVDSKTFFYNVVTNAIFEIDSLIIDILHSKGKNEEHVV |
| | KDLAERYELSQVREAIQNMKEAYIIATDANISDVEKMGILDNSQRVFKLSSLTLFMV |
| | QECNLRCTYCYGEEGEYNQKGKMTSEIARSAVDFLIQQSGEIEQLNITFFGGEPLLNF |
| | PLIQETVQYVHEQSEIHNKKFSFSITTNGTLITPKIKNFFYKHHFAVQTSIDGDEKTHN |
| | FNRFFKGGQGSYDLLLKRTEEMRNDRKIGARGTVTPAELDLSKSFDHLVKLGFRKI |
| | YLSPALYSLSDDHYDTLSKEMVKLVEQFRELLEREDYVTAKKMSNVLGMLSKIHSG |
| | GPRIHFCGAGTNAAAVDVRGNLFPCHRFVGEDECSIGNLFDEDPLSKQYNFIENSTV |
| | RNRTTCSKCWAKNLCGGGCHQENFAENGNVNQPVGKLCKVTKNFINATINLYLQL |
| | TQEQRSILFG (SEQ ID NO: 81) |
| ProcM | MESPSSWKTSWLAAIAPDEPHKFDRRLEWDELSEENFFAALNSEPASLEEDDPCFEE |
| | ALQDALEALKAAWDLPLLPVDNNLNRPFVDVWWPIRCHSAESLRQSFVSDSAGLA |
| | DEIFDQLADSLLDRLCALGDQVLWEAFNKERTPGTMLLAHLGAAGDGSGPPVREH |
| | YERFIQSHRRNGLAPLLKEFPVLGRLIGTVLSLWFQGSVEMLQRICADRTVLQQCFA |
| | IPCGHHLKTVKQGLSDPHRGGRAVAVLEFADPNSTANSSMHVVYKPKDMAVDAA |
| | YQATLADLNTHSDLSPLRTLAIHNGNGYGYMEHVVHHLCANDKELTNFYFNAGRL |
| | TALLHLLGCTDCHHENLIACGDQLLLIDTETLLEADLPDHISDASSTTAQPKPSSLQK |
| | QFQRSVLRSGLLPQWMFLGESKLAIDISALGMSPPNKPERIALGWLGFNSDGMMPG |
| | RVSQPVEIPTSLPVGIGEVNPFDRFLEDFCDGFSMQSEALIKLRNRWLDVNGVLAHF |
| | AGLPRRIVLRATRVYFTIQRQQLEPTALRSPLAQALKLEQLTRSFLLAESKPLHWPIF |
| | AAEVKQMQHLDIPFFTHLIDADALQLGGLEQELPGFIQTSGLAAAYERLRNLDTDEI |
| | AFQLRLIRGAVEARELHTTPESSPTLPPPATPEALMSSSAETSLEAAKRIAHRLLELAI |
| | RDSQGQVEWLGMDLGADGESFSFGPVGLSLYGGSIGIAHLLQRLQAQQVSLMDAD |
| | AIQTAILQPLVGLVDQPSDDGRRRWWRDQPLGLSGCGGTLLALTLQGEQAMANSL |
| | LAAALPRFIEADQQLDLIGGCAGLIGSLVQLGTESALQLALRAGDHLIAQQNEEGA |
| | WSSSSSQPGLLGFSHGTAGYAAALAHLHAFSADERYRTAAAAALAYERARFNKDA |
| | GNWPDYRSIGRDSDSDEPSFMASWCHGAPGIALGRACLWGTALWDEECTKEIGIGL |
| | QTTAAVSSVSTDHLCCGSLGLMVLLEMLSAGPWPIDNQLRSHCQDVAFQYRLQAL |
| | QRCSAEPIKLRCFGTKEGLLVLPGFFTGLSGMGLALLEDDPSRAVVSQLISAGLWPT |
| | E (SEQ ID NO: 82) |
| TgnB | MKTILIITNTLDLTVDYIINRYNHTAKFFRLNTDRFFDYDINITNSGTSIRNRKSNLIINI |
| | QEIHSLYYRKITLPNLDGYESKYWTLMQREMMSIVEGIAETAGNFALTRPSVLRKA |
| | DNKIVQMKLAEEIGFILPQSLITNSNQAAASFCNKNNTSIVKPLSTGRILGKNKIGIIQT |
| | NLVETHENIQGLELSPAYFQDYIPKDTEIRLTIVGNKLFGANIKSTNQVDWRKNDAL |
| | LEYKPANIPDKIAKMCLEMMEKLEINFAAFDFIIRNGDYIFLELNANGQWLWLEDIL |
| | KFDISNTIINYLLGEPI (SEQ ID NO: 83) |

NpuDNAE intein C:
GFIASNCW (SEQ ID NO: 67)
NpuDNAE intein N:
CLSYETEILTVEYGLLPIGKIVEKRIECTVYSVDNNGNIYTQPVAQWHDRGEQEVFEYCLEDGSLIRATKD
HKFMTVDGQMLPIDEIFERELDLMRVDNLPNIKIATRKYLGKQNVYDIGVERDHNFALKN (SEQ ID NO:
68)
ECF20_992 C:
LDTRPAPDEQLEASAQSRRMAQALDQLPDRQREAIVLQYYQELSNTEAAALMQISVEALESLLSRARRN
LRSHLAEAPGADLSGRRKP (SEQ ID NO: 69)
ECF20_992 N:
NETDPDLELLKRIGNNDAQAVKEMVTRKLPRLLALASRLLGDADEARDIAQESFLRIWKQAASWRSEQA
RFDTWLHRVALNLCYDRLRRRKEHVPVDSEHACEA (SEQ ID NO: 70)
SARS-CoV-2 RBD:
RFPNITNLCPFGEVFNATRFASVYAWNRKRISNCVADYSVLYNSASFSTFKCYGVSPTKLNDLCFTNVYA
DSFVIRGDEVRQIAPGQTGKIADYNYKLPDDFTGCVIAWNSNNLDSKVGGNYNYLYRLFRKSNLKPFER
DISTEIYQAGSTPCNGVEGFNCYFPLQSYGFQPTNGVGYQPYRVVVLSFELLHAPATVCGPKKSTNL
(SEQID NO: 71)
ACE2a1:
STIEEQAKTFLDKFNHEAEDLFYQSSLASWNYNTNITE (SEQ ID NO: 72)
lbAMK-101 (plasmid encoding lanthipeptide RiPP library N-terminal to sigma-intein):
tgccacctgacgtctaagaaGAATTCGCGGCCGCTTCTAGAGGGAGccaattattgaaggcctccctaacggggggcctttttttgtttctggtc
tcccgcttaacgatcgttggctgacctgtaggatcgtacaggtTTACGcaagaaaatggtttgtTACAGTcgaataaaagctgtcaccggatgtgctttccggt
ctgatgagtccgtgaggacgaaacagcctctacaaataattttgtttaaTCCATCTCTATGGCGGATTTTatgtcatattaccaccatcaccatcatca
cATGTCAGAAGAACAACTCAAGGCATTCATTGCCAAGGTTCAAGCAGACACTTCACTGCAGGAACA
GCTCAAAGTAGAAGGTGCTGATGTTGTTGCTATTGCTAAAGCCTCAGGGTTCGCGATTACCACAGAG
CTGGCGGAGCTTTCTGAGGAGGCTCTGTCTGATGATGAGCTGGAGGGAGTCGCGGGAGGCGCGGCA
TGCNNKNNKNNKNNKNNKWCGATGCCGCCTWCGNNKNNKNNKNNKNNKTGCCGAggaggtAAGggagg
aCCTggaggtCGGggaggtGTTggaggtGGTggaggaATTggaggtGGTTTTATCGCTTCCAACTGCTGGCTGGATAC
CCGTCCGGCACCGGATGAACAGCTGGAAGCAAGCGCACAGAGCCGTCGTATGGCACAGGCACTGGA
TCAGCTGCCGGATCGTCAGCGTGAAGCAATTGTTCTGCAGTATTATCAAGAACTGAGCAATACCGAA
GCAGCAGCACTGATGCAAATTAGCGTTGAAGCCCTGGAAAGCCTGCTGAGCCGTGCACGTCGTAAT
CTGCGTAGCCATCTGGCCGAAGCACCGGGTGCAGATCTGAGCGGTCGTCGCAAACCGtaaaggtgatactttc
agccaaaaaacttaagaccgccggtcttgtccactaccttgcagtaatgcggtggacaggatcggcggttttcttttctcttctcaaAGACCgTCCAATGGC
GGCGCgccatcgaatggcgcaaaacctttcgcggtatggcatgatagcgcccggaagagagtcaattcagggtggtgaatatgaaaaacataaatgccgacga
cacatacagaataattaataaaattaaagcttgtagaagcaataatgatattaatcaatgcttatctgatatgactaaaatggtacattgtgaatattatttactcgcgatcattt
atcctcattctatggttaaatctgatatttcaatcctagataattaccctaaaaaatggaggcaatattatgatgacgctaatttaataaaatatgatcctatagtagattattcta
actccaatcattcaccaattaattggaatatatttgaaaacaatgctgtaaataaaaaatctccaaatgtaattaaagaagcgaaaacatcaggtcttatcactgggtttagtt
tccctattcatacggctaacaatggcttcggaatgcttagttttgcacattcagaaaaagacaactatatagatagtttatttttacatgcgtgtatgaacataccattaattgtt
ccttctctagttgataattatcgaaaaataaatatagcaaataataaatcaaacaacgatttaaccaaaagagaaaaagaatgtttagcgtgggcatgcgaaggaaaaag
ctcttgggatatttcaaaaatattaggttgcagtgagcgtactgtcactttccatttaaccaatgcgcaaatgaaactcaatacaacaaaccgctgccaaagtatttctaaa
gcaattttaacaggagcaattgattgcccatactttaaaaattgataaggatcctaattggtaacgaatcagacaattgacggctcgagggagtagcatagggtttgcag
aatccctgcttcgtccatttgacaggcacattatgcatcgatgataagctgtcaaacatgagcagatcctctacgccggacgcatcgtggccggcatcaccggcgcca
caggtgcggttgctggcgcctatatcgccgacatcaccgatggggaagatcgggctcgccacttcgggctcatgagcaaatattttatctggctcactcaaaggcggt
aatgacagtaagacgggtaagcctgttgatgataccgctgccttactgggtgcattagccagtctgaatgacctgtcacgggataatccgaagtggtcagactggaaa
atcagagggcaggaactgctgaacagcaaaaagtcagatagcaccacatagcagacccgccataaaacgccctgagaagcccgtgacgggcttttcttgtattatg
ggtagtttccttgcatgaatccataaaaggcgcctgtagtgccatttacccccattcactgccagagccgtgagcgcagcgaactgaatgtcacgaaaaagacagcga
ctcaggtgcctgatggtcggagacaaaaggaatattcagcgatttgcccgagcttgcgagggtgctacttaagcctttagggttttaaggtctgttttgtagaggagcaa
acagcgtttgcgacatccttttgtaatactgcggaactgactaaagtagtgagttatacacagggctgggatctattctttttatctttttttattctttctttattctataaattata
accacttgaatataaacaaaaaaaacacacaaaggtctagcggaatttacagagggtctagcagaatttacaagttttccagcaaaggtctagcagaatttacagatac
ccacaactcaaaggaaaaggactagtaattatcattgactagcccatctcaattggtatagtgattaaaatcacctagaccaattgagatgtatgtctgaattagttgttttc
aaagcaaatgaactagcgattagtcgctatgacttaacggagcatgaaaccaagctaattttatgctgtgtggcactactcaaccccacgattgaaaaccctacaagga
aagaacggacggtatcgttcacttataaccaatacgctcagatgatgaacatcagtagggaaaatgcttatggtgtattagctaaagcaaccagagagctgatgacga
gaactgtggaaatcaggaatcctttggttaaaggctttTGGattttccagtggacaaactatgccaagttctcaagcgaaaaattagaattagtttttagtgaagagatat
tgccttatcttttccagttaaaaaaattcataaaatataatctggaacatgttaagtcttttgaaaacaaatactctatgaggatttatgagtggttattaaaagaactaacaca
aaagaaaactcacaaggcaaatatagagattagccttgatgaatttaagttcatgttaatgcttgaaaataactaccatgagtttaaaaggcttaaccaatgggttttgaaa
ccaataagtaaagatttaaacacttacagcaatatgaaattggtggttgataagcgaggccgcccgactgatacgttgattttccaagttgaactagatagacaaatggat
ctcgtaaccgaacttgagaacaaccagataaaaatgaatggtgacaaaataccaacaaccattacatcagattcctacctacAtaacggactaagaaaaacactacac
gatgctttaactgcaaaaattcagctcaccagttttgaggcaaaatttttgagtgacatgcaaagtaagTatgatctcaatggttcgttctcatggctcacgcaaaaacaa
cgaaccacactagagaacatactggctaaatacggaaggatctgaggttcttatggctcttgtatctatcagtgaagcatcaagactaacaaacaaaagtagaacaact
gttcaccgttaCatatcaaagggaaaactgtccatatgcacagatgaaaacggtgtaaaaaagatagatacatcagagcttttacgagtttttggtgcattCaaagctgt
tcaccatgaacagatcgacaatgtaacagatgaacagcatgtaacacctaatagaacaggtgaaaccagtaaaacaaagcaactagaacatgaaattgaacacctga
gacaacttgttacagctcaacagtcacacatagacagcctgaaacaggcgatgctgcttatcgaatcaaagctgccgacaacacgggagccagtgacgcctcccgt
ggggaaaaaatcatggcaattctggaagaaatagCgctttcagccggcaaacCGGctgaagccggatctgcgattctgataacaaactagcaacaccagaacag
cccgtttgcgggcagcaaaacccgtacCGATTATCAAAAAGGATCTTCACCtagatccttttaaattaaaaatgaagttttaaatcaatctaaagt
atatatgagtaaacttggtctgacagttaccaatgcttaatcagtgaggcacctatctcagcgatctgtctatttcgttcatccatagttgcctgactccccgtcgtgtagata
actacgatacgggagggcttaccatctggccccagtgctgcaatgataccgcgagaAccacgctcaccggctccagatttatcagcaataaaccagccagccgga
agggccgagcgcagaagtggtcctgcaactttatccgcctccatccagtctattaattgttgccgggaagctagagtaagtagttcgccagttaatagtttgcgcaacgt
tgttgccattgctacaggcatcgtggtgtcacgctcgtcgtttggtatggcttcattcagctccggttcccaacgatcaaggcgagttacatgatcccccatgttgtgcaaa
aaagcggttagctccttcggtcctccgatcgttgtcagaagtaagttggccgcagtgttatcactcatggttatggcagcactgcataattctcttactgtcatgccatccgt
aagatgcttttctgtgactggtgagtactcaaccaagtcattctgagaatagtgtatgcggcgaccgagttgctcttgcccggcgtcaatacgggataataccgcgccac
atagcagaactttaaaagtgctcatcattggaaaacgttcttcggggcgaaaactctcaaggatcttaccgctgttgagatccagttcgatAtaacccactcgtgcaccc
aactgatcttcagcatcttttactttcaccagcgtttctgggtgagcaaaaacaggaaggcaaaatgccgcaaaaaagggaataagggcgacacggaaatgttgaata
ctcatactcttcctttttcaatattattgaagcatttatcagggttattgtctcatgagcggatacatatttgaatgtatttagaaaaataaacaaataggggttccgcgcacatt
tccccgaaaag (SEQ ID NO: 73)
lbAMK-102 (plasmid encoding microviridin RiPP library N-terminal to sigma-intein):
tgccacctgacgtctaagaaGAATTCGCGGCCGCTTCTAGAGGGAGccaattattgaaggcctccctaacggggggcctttttttgtttctggtc
tcccgcttaacgatcgttggctgacctgtaggatcgtacaggtTTACGcaagaaaatggtttgtTACAGTcgaataaaagctgtcaccggatgtgctttccggt
ctgatgagtccgtgaggacgaaacagcctctacaaataattttgtttaaTCCATCTCTATGGCGGATTTTatgtcatattaccaccatcaccatcatca
catgTATCGACCTTATATTGCCAAGTATGTCGAAGAACAAACTCTGCAGAATTCAACCAACCTGGTAT
ATGACGACATCACGCAGCTGGCGGAGCTTTCTGAGGAGGCTCTGGTGAAAAAAATTAATCTGNNKC
CCVANACTACGNNKNNKACTNNKDYKNTTGAGNNKNNKGACNNKGATGAGNNKNNKNNKCGAggag
gtAAGggaggaCCTggaggtCGGggaggtGTTggaggtGGTggaggaATTggaggtGGTTTTATCGCTTCCAACTGCTGG
CTGGATACCCGTCCGGCACCGGATGAACAGCTGGAAGCAAGCGCACAGAGCCGTCGTATGGCACAG
GCACTGGATCAGCTGCCGGATCGTCAGCGTGAAGCAATTGTTCTGCAGTATTATCAAGAACTGAGCA
ATACCGAAGCAGCAGCACTGATGCAAATTAGCGTTGAAGCCCTGGAAAGCCTGCTGAGCCGTGCAC
GTCGTAATCTGCGTAGCCATCTGGCCGAAGCACCGGGTGCAGATCTGAGCGGTCGTCGCAAACCGtaa
aggtgatactttcagccaaaaaacttaagaccgccggtcttgtccactaccttgcagtaatgcggtggacaggatcggcggttttcttttctcttctcaaAGACCgT
CCAATGGCGGCGCgccatcgaatggcgcaaaacctttcgcggtatggcatgatagcgcccggaagagagtcaattcagggtggtgaatatgaaaaacat
aaatgccgacgacacatacagaataattaataaaattaaagcttgtagaagcaataatgatattaatcaatgcttatctgatatgactaaaatggtacattgtgaatattattt
actcgcgatcatttatcctcattctatggttaaatctgatatttcaatcctagataattaccctaaaaaatggaggcaatattatgatgacgctaatttaataaaatatgatccta
tagtagattattctaactccaatcattcaccaattaattggaatatatttgaaaacaatgctgtaaataaaaaatctccaaatgtaattaaagaagcgaaaacatcaggtctta
tcactgggtttagtttccctattcatacggctaacaatggcttcggaatgcttagttttgcacattcagaaaaagacaactatatagatagtttatttttacatgcgtgtatgaa
cataccattaattgttccttctctagttgataattatcgaaaaataaatatagcaaataataaatcaaacaacgatttaaccaaaagagaaaaagaatgtttagcgtgggcat
gcgaaggaaaaagctcttgggatatttcaaaaatattaggttgcagtgagcgtactgtcactttccatttaaccaatgcgcaaatgaaactcaatacaacaaaccgctgc
caaagtatttctaaagcaattttaacaggagcaattgattgcccatactttaaaaattgataaggatcctaattggtaacgaatcagacaattgacggctcgagggagtag
catagggtttgcagaatccctgcttcgtccatttgacaggcacattatgcatcgatgataagctgtcaaacatgagcagatcctctacgccggacgcatcgtggccggc
atcaccggcgccacaggtgcggttgctggcgcctatatcgccgacatcaccgatggggaagatcgggctcgccacttcgggctcatgagcaaatattttatctggctc
actcaaaggcggtaatgacagtaagacgggtaagcctgttgatgataccgctgccttactgggtgcattagccagtctgaatgacctgtcacgggataatccgaagtg
gtcagactggaaaatcagagggcaggaactgctgaacagcaaaaagtcagatagcaccacatagcagacccgccataaaacgccctgagaagcccgtgacggg
cttttcttgtattatgggtagtttccttgcatgaatccataaaaggcgcctgtagtgccatttacccccattcactgccagagccgtgagcgcagcgaactgaatgtcacga
aaaagacagcgactcaggtgcctgatggtcggagacaaaaggaatattcagcgatttgcccgagcttgcgagggtgctacttaagcctttagggttttaaggtctgtttt
gtagaggagcaaacagcgtttgcgacatccttttgtaatactgcggaactgactaaagtagtgagttatacacagggctgggatctattctttttatctttttttattctttcttta
ttctataaattataaccacttgaatataaacaaaaaaaacacacaaaggtctagcggaatttacagagggtctagcagaatttacaagttttccagcaaaggtctagcaga
atttacagatacccacaactcaaaggaaaaggactagtaattatcattgactagcccatctcaattggtatagtgattaaaatcacctagaccaattgagatgtatgtctga
attagttgttttcaaagcaaatgaactagcgattagtcgctatgacttaacggagcatgaaaccaagctaattttatgctgtgtggcactactcaaccccacgattgaaaac
cctacaaggaaagaacggacggtatcgttcacttataaccaatacgctcagatgatgaacatcagtagggaaaatgcttatggtgtattagctaaagcaaccagagag
ctgatgacgagaactgtggaaatcaggaatcctttggttaaaggctttTGGattttccagtggacaaactatgccaagttctcaagcgaaaaattagaattagtttttagt
gaagagatattgccttatcttttccagttaaaaaaattcataaaatataatctggaacatgttaagtcttttgaaaacaaatactctatgaggatttatgagtggttattaaaag
aactaacacaaaagaaaactcacaaggcaaatatagagattagccttgatgaatttaagttcatgttaatgcttgaaaataactaccatgagtttaaaaggcttaaccaatg
ggttttgaaaccaataagtaaagatttaaacacttacagcaatatgaaattggtggttgataagcgaggccgcccgactgatacgttgattttccaagttgaactagatag
acaaatggatctcgtaaccgaacttgagaacaaccagataaaaatgaatggtgacaaaataccaacaaccattacatcagattcctacctacAtaacggactaagaaa
aacactacacgatgctttaactgcaaaaattcagctcaccagttttgaggcaaaatttttgagtgacatgcaaagtaagTatgatctcaatggttcgttctcatggctcacg
caaaaacaacgaaccacactagagaacatactggctaaatacggaaggatctgaggttcttatggctcttgtatctatcagtgaagcatcaagactaacaaacaaaagt
agaacaactgttcaccgttaCatatcaaagggaaaactgtccatatgcacagatgaaaacggtgtaaaaaagatagatacatcagagcttttacgagtttttggtgcatt
Caaagctgttcaccatgaacagatcgacaatgtaacagatgaacagcatgtaacacctaatagaacaggtgaaaccagtaaaacaaagcaactagaacatgaaattg
aacacctgagacaacttgttacagctcaacagtcacacatagacagcctgaaacaggcgatgctgcttatcgaatcaaagctgccgacaacacgggagccagtgac
gcctcccgtggggaaaaaatcatggcaattctggaagaaatagCgctttcagccggcaaacCGGctgaagccggatctgcgattctgataacaaactagcaacac
cagaacagcccgtttgcgggcagcaaaacccgtacCGATTATCAAAAAGGATCTTCACCtagatccttttaaattaaaaatgaagttttaaatca
atctaaagtatatatgagtaaacttggtctgacagttaccaatgcttaatcagtgaggcacctatctcagcgatctgtctatttcgttcatccatagttgcctgactccccgtc
gtgtagataactacgatacgggagggcttaccatctggccccagtgctgcaatgataccgcgagaAccacgctcaccggctccagatttatcagcaataaaccagc
cagccggaagggccgagcgcagaagtggtcctgcaactttatccgcctccatccagtctattaattgttgccgggaagctagagtaagtagttcgccagttaatagttt
gcgcaacgttgttgccattgctacaggcatcgtggtgtcacgctcgtcgtttggtatggcttcattcagctccggttcccaacgatcaaggcgagttacatgatcccccat
gttgtgcaaaaaagcggttagctccttcggtcctccgatcgttgtcagaagtaagttggccgcagtgttatcactcatggttatggcagcactgcataattctcttactgtc
atgccatccgtaagatgcttttctgtgactggtgagtactcaaccaagtcattctgagaatagtgtatgcggcgaccgagttgctcttgcccggcgtcaatacgggataat
accgcgccacatagcagaactttaaaagtgctcatcattggaaaacgttcttcggggcgaaaactctcaaggatcttaccgctgttgagatccagttcgatAtaaccca
ctcgtgcacccaactgatcttcagcatcttttactttcaccagcgtttctgggtgagcaaaaacaggaaggcaaaatgccgcaaaaaagggaataagggcgacacgg
aaatgttgaatactcatactcttcctttttcaatattattgaagcatttatcagggttattgtctcatgagcggatacatatttgaatgtatttagaaaaataaacaaataggggtt
ccgcgcacatttccccgaaaag (SEQ ID NO: 74)
lbAMK-103 (plasmid encoding ranthipeptide RiPP library v1 N-terminal to sigma-intein):
tgccacctgacgtctaagaaGAATTCGCGGCCGCTTCTAGAGGGAGccaattattgaaggcctccctaacggggggcctttttttgtttctggtc
tcccgcttaacgatcgttggctgacctgtaggatcgtacaggtTTACGcaagaaaatggtttgtTACAGTcgaataaaagctgtcaccggatgtgctttccggt
ctgatgagtccgtgaggacgaaacagcctctacaaataattttgtttaaTCCATCTCTATGGCGGATTTTatgtcatattaccaccatcaccatcatca
cATGTTGAAACAGATCAATGTGATTGCTGGCGTAAAAGAGCCTATTCGCGCCTATNNKTGTNNKNNK
NNKGAWNNKTGCNNKNNKNNKGAWNNKCGAggaggtAAGggaggaCCTggaggtCGGggaggtGTTggaggtGG
TggaggaATTggaggtGGTTTTATCGCTTCCAACTGCTGGCTGGATACCCGTCCGGCACCGGATGAACAGC
TGGAAGCAAGCGCACAGAGCCGTCGTATGGCACAGGCACTGGATCAGCTGCCGGATCGTCAGCGTG
AAGCAATTGTTCTGCAGTATTATCAAGAACTGAGCAATACCGAAGCAGCAGCACTGATGCAAATTA
GCGTTGAAGCCCTGGAAAGCCTGCTGAGCCGTGCACGTCGTAATCTGCGTAGCCATCTGGCCGAAGC
ACCGGGTGCAGATCTGAGCGGTCGTCGCAAACCGtaaaggtgatactttcagccaaaaaacttaagaccgccggtcttgtccactacc
ttgcagtaatgcggtggacaggatcggcggttttcttttctcttctcaaAGACCgTCCAATGGCGGCGCgccatcgaatggcgcaaaacctttcgcgg
tatggcatgatagcgcccggaagagagtcaattcagggtggtgaatatgaaaaacataaatgccgacgacacatacagaataattaataaaattaaagcttgtagaag
caataatgatattaatcaatgcttatctgatatgactaaaatggtacattgtgaatattatttactcgcgatcatttatcctcattctatggttaaatctgatatttcaatcctagata
attaccctaaaaaatggaggcaatattatgatgacgctaatttaataaaatatgatcctatagtagattattctaactccaatcattcaccaattaattggaatatatttgaaaa
caatgctgtaaataaaaaatctccaaatgtaattaaagaagcgaaaacatcaggtcttatcactgggtttagtttccctattcatacggctaacaatggcttcggaatgctta
gttttgcacattcagaaaaagacaactatatagatagtttatttttacatgcgtgtatgaacataccattaattgttccttctctagttgataattatcgaaaaataaatatagcaa
ataataaatcaaacaacgatttaaccaaaagagaaaaagaatgtttagcgtgggcatgcgaaggaaaaagctcttgggatatttcaaaaatattaggttgcagtgagcg
tactgtcactttccatttaaccaatgcgcaaatgaaactcaatacaacaaaccgctgccaaagtatttctaaagcaattttaacaggagcaattgattgcccatactttaaaa
attgataaggatcctaattggtaacgaatcagacaattgacggctcgagggagtagcatagggtttgcagaatccctgcttcgtccatttgacaggcacattatgcatcg
atgataagctgtcaaacatgagcagatcctctacgccggacgcatcgtggccggcatcaccggcgccacaggtgcggttgctggcgcctatatcgccgacatcacc
gatggggaagatcgggctcgccacttcgggctcatgagcaaatattttatctggctcactcaaaggcggtaatgacagtaagacgggtaagcctgttgatgataccgc
tgccttactgggtgcattagccagtctgaatgacctgtcacgggataatccgaagtggtcagactggaaaatcagagggcaggaactgctgaacagcaaaaagtcag
atagcaccacatagcagacccgccataaaacgccctgagaagcccgtgacgggcttttcttgtattatgggtagtttccttgcatgaatccataaaaggcgcctgtagtg
ccatttacccccattcactgccagagccgtgagcgcagcgaactgaatgtcacgaaaaagacagcgactcaggtgcctgatggtcggagacaaaaggaatattcag
cgatttgcccgagcttgcgagggtgctacttaagcctttagggttttaaggtctgttttgtagaggagcaaacagcgtttgcgacatccttttgtaatactgcggaactgac
taaagtagtgagttatacacagggctgggatctattctttttatctttttttattctttctttattctataaattataaccacttgaatataaacaaaaaaaacacacaaaggtctag
cggaatttacagagggtctagcagaatttacaagttttccagcaaaggtctagcagaatttacagatacccacaactcaaaggaaaaggactagtaattatcattgacta
gcccatctcaattggtatagtgattaaaatcacctagaccaattgagatgtatgtctgaattagttgttttcaaagcaaatgaactagcgattagtcgctatgacttaacgga
gcatgaaaccaagctaattttatgctgtgtggcactactcaaccccacgattgaaaaccctacaaggaaagaacggacggtatcgttcacttataaccaatacgctcag
atgatgaacatcagtagggaaaatgcttatggtgtattagctaaagcaaccagagagctgatgacgagaactgtggaaatcaggaatcctttggttaaaggctttTGG
attttccagtggacaaactatgccaagttctcaagcgaaaaattagaattagtttttagtgaagagatattgccttatcttttccagttaaaaaaattcataaaatataatctgg
aacatgttaagtcttttgaaaacaaatactctatgaggatttatgagtggttattaaaagaactaacacaaaagaaaactcacaaggcaaatatagagattagccttgatga
atttaagttcatgttaatgcttgaaaataactaccatgagtttaaaaggcttaaccaatgggttttgaaaccaataagtaaagatttaaacacttacagcaatatgaaattggt
ggttgataagcgaggccgcccgactgatacgttgattttccaagttgaactagatagacaaatggatctcgtaaccgaacttgagaacaaccagataaaaatgaatggt
gacaaaataccaacaaccattacatcagattcctacctacAtaacggactaagaaaaacactacacgatgctttaactgcaaaaattcagctcaccagttttgaggcaa
aatttttgagtgacatgcaaagtaagTatgatctcaatggttcgttctcatggctcacgcaaaaacaacgaaccacactagagaacatactggctaaatacggaaggat
ctgaggttcttatggctcttgtatctatcagtgaagcatcaagactaacaaacaaaagtagaacaactgttcaccgttaCatatcaaagggaaaactgtccatatgcaca
gatgaaaacggtgtaaaaaagatagatacatcagagcttttacgagtttttggtgcattCaaagctgttcaccatgaacagatcgacaatgtaacagatgaacagcatgt
aacacctaatagaacaggtgaaaccagtaaaacaaagcaactagaacatgaaattgaacacctgagacaacttgttacagctcaacagtcacacatagacagcctga
aacaggcgatgctgcttatcgaatcaaagctgccgacaacacgggagccagtgacgcctcccgtggggaaaaaatcatggcaattctggaagaaatagCgctttca
gccggcaaacCGGctgaagccggatctgcgattctgataacaaactagcaacaccagaacagcccgtttgcgggcagcaaaacccgtacCGATTATCA
AAAAGGATCTTCACCtagatccttttaaattaaaaatgaagttttaaatcaatctaaagtatatatgagtaaacttggtctgacagttaccaatgcttaatcagt
gaggcacctatctcagcgatctgtctatttcgttcatccatagttgcctgactccccgtcgtgtagataactacgatacgggagggcttaccatctggccccagtgctgca
atgataccgcgagaAccacgctcaccggctccagatttatcagcaataaaccagccagccggaagggccgagcgcagaagtggtcctgcaactttatccgcctcc
atccagtctattaattgttgccgggaagctagagtaagtagttcgccagttaatagtttgcgcaacgttgttgccattgctacaggcatcgtggtgtcacgctcgtcgtttg
gtatggcttcattcagctccggttcccaacgatcaaggcgagttacatgatcccccatgttgtgcaaaaaagcggttagctccttcggtcctccgatcgttgtcagaagta
agttggccgcagtgttatcactcatggttatggcagcactgcataattctcttactgtcatgccatccgtaagatgcttttctgtgactggtgagtactcaaccaagtcattct
gagaatagtgtatgcggcgaccgagttgctcttgcccggcgtcaatacgggataataccgcgccacatagcagaactttaaaagtgctcatcattggaaaacgttcttc
ggggcgaaaactctcaaggatcttaccgctgttgagatccagttcgatAtaacccactcgtgcacccaactgatcttcagcatcttttactttcaccagcgtttctgggtg
agcaaaaacaggaaggcaaaatgccgcaaaaaagggaataagggcgacacggaaatgttgaatactcatactcttcctttttcaatattattgaagcatttatcagggtt
attgtctcatgagcggatacatatttgaatgtatttagaaaaataaacaaataggggttccgcgcacatttccccgaaaag (SEQ ID NO: 75)
lbAMK-104 (plasmid encoding ranthipeptide RiPP library v2 N-terminal to sigma-intein):
tgccacctgacgtctaagaaGAATTCGCGGCCGCTTCTAGAGGGAGccaattattgaaggcctccctaacggggggcctttttttgtttctggtc
tcccgcttaacgatcgttggctgacctgtaggatcgtacaggtTTACGcaagaaaatggtttgtTACAGTcgaataaaagctgtcaccggatgtgctttccggt
ctgatgagtccgtgaggacgaaacagcctctacaaataattttgtttaaTCCATCTCTATGGCGGATTTTatgtcatattaccaccatcaccatcatca
cATGTTGAAACAGATCAATGTGATTGCTGGCGTAAAAGAGCCTATTCGCGCCTATNNKTGCNNKNNK
TGCGAWNNKNNKGAWNNKNNKCGAggaggtAAGggaggaCCTggaggtCGGggaggtGTTggaggtGGTggaggaAT
TggaggtGGTTTTATCGCTTCCAACTGCTGGCTGGATACCCGTCCGGCACCGGATGAACAGCTGGAAGC
AAGCGCACAGAGCCGTCGTATGGCACAGGCACTGGATCAGCTGCCGGATCGTCAGCGTGAAGCAAT
TGTTCTGCAGTATTATCAAGAACTGAGCAATACCGAAGCAGCAGCACTGATGCAAATTAGCGTTGA
AGCCCTGGAAAGCCTGCTGAGCCGTGCACGTCGTAATCTGCGTAGCCATCTGGCCGAAGCACCGGG
TGCAGATCTGAGCGGTCGTCGCAAACCGtaaaggtgatactttcagccaaaaaacttaagaccgccggtcttgtccactaccttgcagtaat
gcggtggacaggatcggcggttttcttttctcttctcaaAGACCgTCCAATGGCGGCGCgccatcgaatggcgcaaaacctttcgcggtatggcatga
tagcgcccggaagagagtcaattcagggtggtgaatatgaaaaacataaatgccgacgacacatacagaataattaataaaattaaagcttgtagaagcaataatgat
attaatcaatgcttatctgatatgactaaaatggtacattgtgaatattatttactcgcgatcatttatcctcattctatggttaaatctgatatttcaatcctagataattaccctaa
aaaatggaggcaatattatgatgacgctaatttaataaaatatgatcctatagtagattattctaactccaatcattcaccaattaattggaatatatttgaaaacaatgctgta
aataaaaaatctccaaatgtaattaaagaagcgaaaacatcaggtcttatcactgggtttagtttccctattcatacggctaacaatggcttcggaatgcttagttttgcaca
ttcagaaaaagacaactatatagatagtttatttttacatgcgtgtatgaacataccattaattgttccttctctagttgataattatcgaaaaataaatatagcaaataataaat
caaacaacgatttaaccaaaagagaaaaagaatgtttagcgtgggcatgcgaaggaaaaagctcttgggatatttcaaaaatattaggttgcagtgagcgtactgtcac
tttccatttaaccaatgcgcaaatgaaactcaatacaacaaaccgctgccaaagtatttctaaagcaattttaacaggagcaattgattgcccatactttaaaaattgataag
gatcctaattggtaacgaatcagacaattgacggctcgagggagtagcatagggtttgcagaatccctgcttcgtccatttgacaggcacattatgcatcgatgataagc
tgtcaaacatgagcagatcctctacgccggacgcatcgtggccggcatcaccggcgccacaggtgcggttgctggcgcctatatcgccgacatcaccgatgggga
agatcgggctcgccacttcgggctcatgagcaaatattttatctggctcactcaaaggcggtaatgacagtaagacgggtaagcctgttgatgataccgctgccttactg
ggtgcattagccagtctgaatgacctgtcacgggataatccgaagtggtcagactggaaaatcagagggcaggaactgctgaacagcaaaaagtcagatagcacca
catagcagacccgccataaaacgccctgagaagcccgtgacgggcttttcttgtattatgggtagtttccttgcatgaatccataaaaggcgcctgtagtgccatttacc
cccattcactgccagagccgtgagcgcagcgaactgaatgtcacgaaaaagacagcgactcaggtgcctgatggtcggagacaaaaggaatattcagcgatttgcc
cgagcttgcgagggtgctacttaagcctttagggttttaaggtctgttttgtagaggagcaaacagcgtttgcgacatccttttgtaatactgcggaactgactaaagtagt
gagttatacacagggctgggatctattctttttatctttttttattctttctttattctataaattataaccacttgaatataaacaaaaaaaacacacaaaggtctagcggaattta
cagagggtctagcagaatttacaagttttccagcaaaggtctagcagaatttacagatacccacaactcaaaggaaaaggactagtaattatcattgactagcccatctc
aattggtatagtgattaaaatcacctagaccaattgagatgtatgtctgaattagttgttttcaaagcaaatgaactagcgattagtcgctatgacttaacggagcatgaaac
caagctaattttatgctgtgtggcactactcaaccccacgattgaaaaccctacaaggaaagaacggacggtatcgttcacttataaccaatacgctcagatgatgaaca
tcagtagggaaaatgcttatggtgtattagctaaagcaaccagagagctgatgacgagaactgtggaaatcaggaatcctttggttaaaggctttTGGattttccagtg
gacaaactatgccaagttctcaagcgaaaaattagaattagtttttagtgaagagatattgccttatcttttccagttaaaaaaattcataaaatataatctggaacatgttaa
gtcttttgaaaacaaatactctatgaggatttatgagtggttattaaaagaactaacacaaaagaaaactcacaaggcaaatatagagattagccttgatgaatttaagttc
atgttaatgcttgaaaataactaccatgagtttaaaaggcttaaccaatgggttttgaaaccaataagtaaagatttaaacacttacagcaatatgaaattggtggttgataa
gcgaggccgcccgactgatacgttgattttccaagttgaactagatagacaaatggatctcgtaaccgaacttgagaacaaccagataaaaatgaatggtgacaaaat
accaacaaccattacatcagattcctacctacAtaacggactaagaaaaacactacacgatgctttaactgcaaaaattcagctcaccagttttgaggcaaaatttttgag
tgacatgcaaagtaagTatgatctcaatggttcgttctcatggctcacgcaaaaacaacgaaccacactagagaacatactggctaaatacggaaggatctgaggttc
ttatggctcttgtatctatcagtgaagcatcaagactaacaaacaaaagtagaacaactgttcaccgttaCatatcaaagggaaaactgtccatatgcacagatgaaaac
ggtgtaaaaaagatagatacatcagagcttttacgagtttttggtgcattCaaagctgttcaccatgaacagatcgacaatgtaacagatgaacagcatgtaacacctaa
tagaacaggtgaaaccagtaaaacaaagcaactagaacatgaaattgaacacctgagacaacttgttacagctcaacagtcacacatagacagcctgaaacaggcg
atgctgcttatcgaatcaaagctgccgacaacacgggagccagtgacgcctcccgtggggaaaaaatcatggcaattctggaagaaatagCgctttcagccggcaa
acCGGctgaagccggatctgcgattctgataacaaactagcaacaccagaacagcccgtttgcgggcagcaaaacccgtacCGATTATCAAAAAG
GATCTTCACCtagatccttttaaattaaaaatgaagttttaaatcaatctaaagtatatatgagtaaacttggtctgacagttaccaatgcttaatcagtgaggcacc
tatctcagcgatctgtctatttcgttcatccatagttgcctgactccccgtcgtgtagataactacgatacgggagggcttaccatctggccccagtgctgcaatgataccg
cgagaAccacgctcaccggctccagatttatcagcaataaaccagccagccggaagggccgagcgcagaagtggtcctgcaactttatccgcctccatccagtcta
ttaattgttgccgggaagctagagtaagtagttcgccagttaatagtttgcgcaacgttgttgccattgctacaggcatcgtggtgtcacgctcgtcgtttggtatggcttca
ttcagctccggttcccaacgatcaaggcgagttacatgatcccccatgttgtgcaaaaaagcggttagctccttcggtcctccgatcgttgtcagaagtaagttggccgc
agtgttatcactcatggttatggcagcactgcataattctcttactgtcatgccatccgtaagatgcttttctgtgactggtgagtactcaaccaagtcattctgagaatagtg
tatgcggcgaccgagttgctcttgcccggcgtcaatacgggataataccgcgccacatagcagaactttaaaagtgctcatcattggaaaacgttcttcggggcgaaa
actctcaaggatcttaccgctgttgagatccagttcgatAtaacccactcgtgcacccaactgatcttcagcatcttttactttcaccagcgtttctgggtgagcaaaaaca
ggaaggcaaaatgccgcaaaaaagggaataagggcgacacggaaatgttgaatactcatactcttcctttttcaatattattgaagcatttatcagggttattgtctcatga
gcggatacatatttgaatgtatttagaaaaataaacaaataggggttccgcgcacatttccccgaaaag (SEQ ID NO: 76)
lbAMK-105 (plasmid encoding ranthipeptide RiPP library v3 N-terminal to sigma-intein):
tgccacctgacgtctaagaaGAATTCGCGGCCGCTTCTAGAGGGAGccaattattgaaggcctccctaacggggggcctttttttgtttctggtc
tcccgcttaacgatcgttggctgacctgtaggatcgtacaggtTTACGcaagaaaatggtttgtTACAGTcgaataaaagctgtcaccggatgtgctttccggt
ctgatgagtccgtgaggacgaaacagcctctacaaataattttgtttaaTCCATCTCTATGGCGGATTTTatgtcatattaccaccatcaccatcatca
cATGTTGAAACAGATCAATGTGATTGCTGGCGTAAAAGAGCCTATTCGCGCCTATNNKTGTNNKNNK
NNKGAWNNKTGCNNKNNKTGCGAWNNKNNKGAWNNKCGAggaggtAAGggaggaCCTggaggtCGGggaggt
GTTggaggtGGTggaggaATTggaggtGGTTTTATCGCTTCCAACTGCTGGCTGGATACCCGTCCGGCACCGG
ATGAACAGCTGGAAGCAAGCGCACAGAGCCGTCGTATGGCACAGGCACTGGATCAGCTGCCGGATC
GTCAGCGTGAAGCAATTGTTCTGCAGTATTATCAAGAACTGAGCAATACCGAAGCAGCAGCACTGA
TGCAAATTAGCGTTGAAGCCCTGGAAAGCCTGCTGAGCCGTGCACGTCGTAATCTGCGTAGCCATCT
GGCCGAAGCACCGGGTGCAGATCTGAGCGGTCGTCGCAAACCGtaaaggtgatactttcagccaaaaaacttaagaccgcc
ggtcttgtccactaccttgcagtaatgcggtggacaggatcggcggttttcttttctcttctcaaAGACCgTCCAATGGCGGCGCgccatcgaatggcg
caaaacctttcgcggtatggcatgatagcgcccggaagagagtcaattcagggtggtgaatatgaaaaacataaatgccgacgacacatacagaataattaataaaat
taaagcttgtagaagcaataatgatattaatcaatgcttatctgatatgactaaaatggtacattgtgaatattatttactcgcgatcatttatcctcattctatggttaaatctga
tatttcaatcctagataattaccctaaaaaatggaggcaatattatgatgacgctaatttaataaaatatgatcctatagtagattattctaactccaatcattcaccaattaatt
ggaatatatttgaaaacaatgctgtaaataaaaaatctccaaatgtaattaaagaagcgaaaacatcaggtcttatcactgggtttagtttccctattcatacggctaacaat
ggcttcggaatgcttagttttgcacattcagaaaaagacaactatatagatagtttatttttacatgcgtgtatgaacataccattaattgttccttctctagttgataattatcga
aaaataaatatagcaaataataaatcaaacaacgatttaaccaaaagagaaaaagaatgtttagcgtgggcatgcgaaggaaaaagctcttgggatatttcaaaaatatt
aggttgcagtgagcgtactgtcactttccatttaaccaatgcgcaaatgaaactcaatacaacaaaccgctgccaaagtatttctaaagcaattttaacaggagcaattga
ttgcccatactttaaaaattgataaggatcctaattggtaacgaatcagacaattgacggctcgagggagtagcatagggtttgcagaatccctgcttcgtccatttgaca
ggcacattatgcatcgatgataagctgtcaaacatgagcagatcctctacgccggacgcatcgtggccggcatcaccggcgccacaggtgcggttgctggcgcctat
atcgccgacatcaccgatggggaagatcgggctcgccacttcgggctcatgagcaaatattttatctggctcactcaaaggcggtaatgacagtaagacgggtaagc
ctgttgatgataccgctgccttactgggtgcattagccagtctgaatgacctgtcacgggataatccgaagtggtcagactggaaaatcagagggcaggaactgctga
acagcaaaaagtcagatagcaccacatagcagacccgccataaaacgccctgagaagcccgtgacgggcttttcttgtattatgggtagtttccttgcatgaatccata
aaaggcgcctgtagtgccatttacccccattcactgccagagccgtgagcgcagcgaactgaatgtcacgaaaaagacagcgactcaggtgcctgatggtcggaga
caaaaggaatattcagcgatttgcccgagcttgcgagggtgctacttaagcctttagggttttaaggtctgttttgtagaggagcaaacagcgtttgcgacatccttttgta
atactgcggaactgactaaagtagtgagttatacacagggctgggatctattctttttatctttttttattctttctttattctataaattataaccacttgaatataaacaaaaaaa
acacacaaaggtctagcggaatttacagagggtctagcagaatttacaagttttccagcaaaggtctagcagaatttacagatacccacaactcaaaggaaaaggact
agtaattatcattgactagcccatctcaattggtatagtgattaaaatcacctagaccaattgagatgtatgtctgaattagttgttttcaaagcaaatgaactagcgattagt
cgctatgacttaacggagcatgaaaccaagctaattttatgctgtgtggcactactcaaccccacgattgaaaaccctacaaggaaagaacggacggtatcgttcactt
ataaccaatacgctcagatgatgaacatcagtagggaaaatgcttatggtgtattagctaaagcaaccagagagctgatgacgagaactgtggaaatcaggaatccttt
ggttaaaggctttTGGattttccagtggacaaactatgccaagttctcaagcgaaaaattagaattagtttttagtgaagagatattgccttatcttttccagttaaaaaaat
tcataaaatataatctggaacatgttaagtcttttgaaaacaaatactctatgaggatttatgagtggttattaaaagaactaacacaaaagaaaactcacaaggcaaatat
agagattagccttgatgaatttaagttcatgttaatgcttgaaaataactaccatgagtttaaaaggcttaaccaatgggttttgaaaccaataagtaaagatttaaacactta
cagcaatatgaaattggtggttgataagcgaggccgcccgactgatacgttgattttccaagttgaactagatagacaaatggatctcgtaaccgaacttgagaacaac
cagataaaaatgaatggtgacaaaataccaacaaccattacatcagattcctacctacAtaacggactaagaaaaacactacacgatgctttaactgcaaaaattcagc
tcaccagttttgaggcaaaatttttgagtgacatgcaaagtaagTatgatctcaatggttcgttctcatggctcacgcaaaaacaacgaaccacactagagaacatactg
gctaaatacggaaggatctgaggttcttatggctcttgtatctatcagtgaagcatcaagactaacaaacaaaagtagaacaactgttcaccgttaCatatcaaagggaa
aactgtccatatgcacagatgaaaacggtgtaaaaaagatagatacatcagagcttttacgagtttttggtgcattCaaagctgttcaccatgaacagatcgacaatgta
acagatgaacagcatgtaacacctaatagaacaggtgaaaccagtaaaacaaagcaactagaacatgaaattgaacacctgagacaacttgttacagctcaacagtc
acacatagacagcctgaaacaggcgatgctgcttatcgaatcaaagctgccgacaacacgggagccagtgacgcctcccgtggggaaaaaatcatggcaattctgg
aagaaatagCgctttcagccggcaaacCGGctgaagccggatctgcgattctgataacaaactagcaacaccagaacagcccgtttgcgggcagcaaaacccgt
acCGATTATCAAAAAGGATCTTCACCtagatccttttaaattaaaaatgaagttttaaatcaatctaaagtatatatgagtaaacttggtctgacagtt
accaatgcttaatcagtgaggcacctatctcagcgatctgtctatttcgttcatccatagttgcctgactccccgtcgtgtagataactacgatacgggagggcttaccatc
tggccccagtgctgcaatgataccgcgagaAccacgctcaccggctccagatttatcagcaataaaccagccagccggaagggccgagcgcagaagtggtcctg
caactttatccgcctccatccagtctattaattgttgccgggaagctagagtaagtagttcgccagttaatagtttgcgcaacgttgttgccattgctacaggcatcgtggt
gtcacgctcgtcgtttggtatggcttcattcagctccggttcccaacgatcaaggcgagttacatgatcccccatgttgtgcaaaaaagcggttagctccttcggtcctcc
gatcgttgtcagaagtaagttggccgcagtgttatcactcatggttatggcagcactgcataattctcttactgtcatgccatccgtaagatgcttttctgtgactggtgagt
actcaaccaagtcattctgagaatagtgtatgcggcgaccgagttgctcttgcccggcgtcaatacgggataataccgcgccacatagcagaactttaaaagtgctcat
cattggaaaacgttcttcggggcgaaaactctcaaggatcttaccgctgttgagatccagttcgatAtaacccactcgtgcacccaactgatcttcagcatcttttactttc
accagcgtttctgggtgagcaaaaacaggaaggcaaaatgccgcaaaaaagggaataagggcgacacggaaatgttgaatactcatactcttcctttttcaatattatt
gaagcatttatcagggttattgtctcatgagcggatacatatttgaatgtatttagaaaaataaacaaataggggttccgcgcacatttccccgaaaag (SEQ ID
NO: 77)
pAMK-857 (plasmid encoding ACE2a1 N-terminal to sigma-intein):
tgccacctgacgtctaagaaGAATTCGCGGCCGCTTCTAGAGGGAGccaattattgaaggcctccctaacggggggcctttttttgtttctggtc
tcccgcttaacgatcgttggctgacctgtaggatcgtacaggtTTACGcaagaaaatggtttgtTACAGTcgaataaaagctgtcaccggatgtgctttccggt
ctgatgagtccgtgaggacgaaacagcctctacaaataattttgtttaaTCCATCTCTATGGCGGATTTTatgtcatattaccaccatcaccatcatca
cATGtcaacgatcgaagaacaggctaaaacgttcctggataagttcaatcatgaggcggaggacctgttctaccaaagcagcttggcctcttggaactacaacacg
aacattacggagCGAggaggtAAGggaggaCCTggaggtCGGggaggtGTTggaggtGGTggaggaATTggaggtGGTTTTATCG
CTTCCAACTGCTGGCTGGATACCCGTCCGGCACCGGATGAACAGCTGGAAGCAAGCGCACAGAGCC
GTCGTATGGCACAGGCACTGGATCAGCTGCCGGATCGTCAGCGTGAAGCAATTGTTCTGCAGTATTA
TCAAGAACTGAGCAATACCGAAGCAGCAGCACTGATGCAAATTAGCGTTGAAGCCCTGGAAAGCCT
GCTGAGCCGTGCACGTCGTAATCTGCGTAGCCATCTGGCCGAAGCACCGGGTGCAGATCTGAGCGG
TCGTCGCAAACCGtaaaggtgatactttcagccaaaaaacttaagaccgccggtcttgtccactaccttgcagtaatgcggtggacaggatcggcggttttc
ttttctcttctcaaAGACCgTCCAATGGCGGCGCgccatcgaatggcgcaaaacctttcgcggtatggcatgatagcgcccggaagagagtcaattc
agggtggtgaatatgaaaaacataaatgccgacgacacatacagaataattaataaaattaaagcttgtagaagcaataatgatattaatcaatgcttatctgatatgacta
aaatggtacattgtgaatattatttactcgcgatcatttatcctcattctatggttaaatctgatatttcaatcctagataattaccctaaaaaatggaggcaatattatgatgac
gctaatttaataaaatatgatcctatagtagattattctaactccaatcattcaccaattaattggaatatatttgaaaacaatgctgtaaataaaaaatctccaaatgtaattaa
agaagcgaaaacatcaggtcttatcactgggtttagtttccctattcatacggctaacaatggcttcggaatgcttagttttgcacattcagaaaaagacaactatatagat
agtttatttttacatgcgtgtatgaacataccattaattgttccttctctagttgataattatcgaaaaataaatatagcaaataataaatcaaacaacgatttaaccaaaagag
aaaaagaatgtttagcgtgggcatgcgaaggaaaaagctcttgggatatttcaaaaatattaggttgcagtgagcgtactgtcactttccatttaaccaatgcgcaaatga
aactcaatacaacaaaccgctgccaaagtatttctaaagcaattttaacaggagcaattgattgcccatactttaaaaattgataaggatcctaattggtaacgaatcagac
aattgacggctcgagggagtagcatagggtttgcagaatccctgcttcgtccatttgacaggcacattatgcatcgatgataagctgtcaaacatgagcagatcctctac
gccggacgcatcgtggccggcatcaccggcgccacaggtgcggttgctggcgcctatatcgccgacatcaccgatggggaagatcgggctcgccacttcgggctc
atgagcaaatattttatctggctcactcaaaggcggtaatgacagtaagacgggtaagcctgttgatgataccgctgccttactgggtgcattagccagtctgaatgacc
tgtcacgggataatccgaagtggtcagactggaaaatcagagggcaggaactgctgaacagcaaaaagtcagatagcaccacatagcagacccgccataaaacgc
cctgagaagcccgtgacgggcttttcttgtattatgggtagtttccttgcatgaatccataaaaggcgcctgtagtgccatttacccccattcactgccagagccgtgagc
gcagcgaactgaatgtcacgaaaaagacagcgactcaggtgcctgatggtcggagacaaaaggaatattcagcgatttgcccgagcttgcgagggtgctacttaag
cctttagggttttaaggtctgttttgtagaggagcaaacagcgtttgcgacatccttttgtaatactgcggaactgactaaagtagtgagttatacacagggctgggatcta
ttctttttatctttttttattctttctttattctataaattataaccacttgaatataaacaaaaaaaacacacaaaggtctagcggaatttacagagggtctagcagaatttacaag
ttttccagcaaaggtctagcagaatttacagatacccacaactcaaaggaaaaggactagtaattatcattgactagcccatctcaattggtatagtgattaaaatcaccta
gaccaattgagatgtatgtctgaattagttgttttcaaagcaaatgaactagcgattagtcgctatgacttaacggagcatgaaaccaagctaattttatgctgtgtggcact
actcaaccccacgattgaaaaccctacaaggaaagaacggacggtatcgttcacttataaccaatacgctcagatgatgaacatcagtagggaaaatgcttatggtgta
ttagctaaagcaaccagagagctgatgacgagaactgtggaaatcaggaatcctttggttaaaggctttTGGattttccagtggacaaactatgccaagttctcaagc
gaaaaattagaattagtttttagtgaagagatattgccttatcttttccagttaaaaaaattcataaaatataatctggaacatgttaagtcttttgaaaacaaatactctatgag
gatttatgagtggttattaaaagaactaacacaaaagaaaactcacaaggcaaatatagagattagccttgatgaatttaagttcatgttaatgcttgaaaataactaccat
gagtttaaaaggcttaaccaatgggttttgaaaccaataagtaaagatttaaacacttacagcaatatgaaattggtggttgataagcgaggccgcccgactgatacgtt
gattttccaagttgaactagatagacaaatggatctcgtaaccgaacttgagaacaaccagataaaaatgaatggtgacaaaataccaacaaccattacatcagattcct
acctacAtaacggactaagaaaaacactacacgatgctttaactgcaaaaattcagctcaccagttttgaggcaaaatttttgagtgacatgcaaagtaagTatgatctc
aatggttcgttctcatggctcacgcaaaaacaacgaaccacactagagaacatactggctaaatacggaaggatctgaggttcttatggctcttgtatctatcagtgaag
catcaagactaacaaacaaaagtagaacaactgttcaccgttaCatatcaaagggaaaactgtccatatgcacagatgaaaacggtgtaaaaaagatagatacatcag
agcttttacgagtttttggtgcattCaaagctgttcaccatgaacagatcgacaatgtaacagatgaacagcatgtaacacctaatagaacaggtgaaaccagtaaaac
aaagcaactagaacatgaaattgaacacctgagacaacttgttacagctcaacagtcacacatagacagcctgaaacaggcgatgctgcttatcgaatcaaagctgcc
gacaacacgggagccagtgacgcctcccgtggggaaaaaatcatggcaattctggaagaaatagCgctttcagccggcaaacCGGctgaagccggatctgcg
attctgataacaaactagcaacaccagaacagcccgtttgcgggcagcaaaacccgtacCGATTATCAAAAAGGATCTTCACCtagatcctttt
aaattaaaaatgaagttttaaatcaatctaaagtatatatgagtaaacttggtctgacagttaccaatgcttaatcagtgaggcacctatctcagcgatctgtctatttcgttca
tccatagttgcctgactccccgtcgtgtagataactacgatacgggagggcttaccatctggccccagtgctgcaatgataccgcgagaAccacgctcaccggctcc
agatttatcagcaataaaccagccagccggaagggccgagcgcagaagtggtcctgcaactttatccgcctccatccagtctattaattgttgccgggaagctagagt
aagtagttcgccagttaatagtttgcgcaacgttgttgccattgctacaggcatcgtggtgtcacgctcgtcgtttggtatggcttcattcagctccggttcccaacgatca
aggcgagttacatgatcccccatgttgtgcaaaaaagcggttagctccttcggtcctccgatcgttgtcagaagtaagttggccgcagtgttatcactcatggttatggca
gcactgcataattctcttactgtcatgccatccgtaagatgcttttctgtgactggtgagtactcaaccaagtcattctgagaatagtgtatgcggcgaccgagttgctcttg
cccggcgtcaatacgggataataccgcgccacatagcagaactttaaaagtgctcatcattggaaaacgttcttcggggcgaaaactctcaaggatcttaccgctgttg
agatccagttcgatAtaacccactcgtgcacccaactgatcttcagcatcttttactttcaccagcgtttctgggtgagcaaaaacaggaaggcaaaatgccgcaaaaa
agggaataagggcgacacggaaatgttgaatactcatactcttcctttttcaatattattgaagcatttatcagggttattgtctcatgagcggatacatatttgaatgtattta
gaaaaataaacaaataggggttccgcgcacatttccccgaaaag (SEQ ID NO: 78)
pAMK-876 (plasmid encoding RBD C-terminal to sigma-intein; ECF promoter driving expression of cat-GFP and
hsvtk-RFP):
cgattatcaaaaaggatcttcacctagatccttttaaattaaaaatgaagttttaaatcaatctaaagtatatatgagtaaacttggtctgacagttatcagaagaactcgtca
agaaggcgatagaaggcgatgcgctgcgaatcgggagcggcgataccgtaaagcacgaggaagcggtcagcccattcgccgccaagctcttcagcaatatcacg
ggtagccaacgctatgtcctgatagcggtccgccacacccagccggccacagtcgatgaatccagaaaagcggccattttccaccatgatattcggcaagcaggcat
cgccatgggtcacgacgagatcctcgccgtcgggcatgcgcgccttgagcctggcgaacagttcggctggcgcgagcccctgatgctcttcgtccagatcatcctg
atcgacaagaccggcttccatccgagtacgtgctcgctcgatgcgatgtttcgcttggtggtcgaatgggcaggtagccggatcaagcgtatgcagccgccgcattg
catcagccatgatggatactttctcggcaggagcaaggtgagatgacaggagatcctgccccggcacttcgcccaatagcagccagtcccttcccgcttcagtgaca
acgtcgagcacagctgcgcaaggaacgcccgtcgtggccagccacgatagccgcgctgcctcgtcctgcagttcattcagggcaccggacaggtcggtcttgaca
aaaagaaccgggcgcccctgcgctgacagccggaacacggcggcatcagagcagccgattgtctgttgtgcccagtcatagccgaatagcctctccacccaagcg
gccggagaacctgcgtgcaatccatcttgttcaatcatgcgaaacgatcctcaatattattgaagcatttatcagggttattgtctcatgagcggatacatatttgaatgtatt
tagaaaaataaacaaataggggttccgcgcacatttccccgaaaagtgccacctgacgtctaagaaaccattattatcatgacattaacctataaaaataggcgtatcac
gaggcagaatttcagataaaaaaaatccttagctttcgctaaggatgatttctggaattcgcggccgcttctagagGGAGgcgcggataaaaatttcatttgcccgc
GACGGATtccccgcccatctatCGTTGAAcccatcagctgcgttcatcagcgaAGctgtcaccggatgtgctttccggtctgatgagtccgtgaggacg
aaacagcctctacaaataattttgtttaaTACTtcacacaggaaagtactagATGAGCAAAGGAGAAGAACTTTTCACTGGAGTTG
TCCCAATTCTTGTTGAATTAGATGGTGATGTTAATGGGCACAAATTTTCTGTCCGTGGAGAGGGTGA
AGGTGATGCTACAAACGGAAAACTCACCCTTAAATTTATTTGCACTACTGGAAAACTACCTGTTCCG
TGGCCAACACTTGTCACTACTCTGACCTATGGTGTTCAATGCTTTTCCCGTTATCCGGATCACATGAA
ACGGCATGACTTTTTCAAGAGTGCCATGCCCGAAGGTTATGTACAGGAACGCACTATATCTTTCAAA
GATGACGGGACCTACAAGACGCGTGCTGAAGTCAAGTTTGAAGGTGATACCCTTGTTAATCGTATCG
AGTTAAAGGGTATTGATTTTAAAGAAGATGGAAACATTCTTGGACACAAACTCGAGTACAACTTTAA
CTCACACAATGTATACATCACGGCAGACAAACAAAAGAATGGAATCAAAGCTAACTTCAAAATTCG
CCACAACGTTGAAGATGGTTCCGTTCAACTAGCAGACCATTATCAACAAAATACTCCAATTGGCGAT
GGCCCTGTCCTTTTACCAGACAACCATTACCTGTCGACACAATCTGTCCTTTCGAAAGATCCCAACG
AAAAGCGTGACCACATGGTCCTTCTTGAGTTTGTAACTGCTGCTGGGATTACACATGGCATGGATGA
GCTCTACAAAggaggtgagaaaaaaatcactggatataccaccgttgatatatcccaatggcatcgtaaagaacattttgaggcatttcagtcagttgctcaat
gtacctataaccagaccgttcagctggatattacggcctttttaaagaccgtaaagaaaaataagcacaagttttatccggcctttattcacattcttgcccgcctgatgaat
gctcatccggaatttcgtatggcaatgaaagacggtgagctggtgatatgggatagtgttcacccttgttacaccgttttccatgagcaaactgaaacgttttcatcgctct
ggagtgaataccacgacgatttccggcagtttctacacatatattcgcaagatgtggcgtgttacggtgaaaacctggcctatttccctaaagggtttattgagaatatgtt
tttcgtctcagccaatccctgggtgagtttcaccagttttgatttaaacgtggccaatatggacaacttcttcgcccccgttttcaccatgggcaaatattatacgcaaggcg
acaaggtgctgatgccgctggcgattcaggttcatcatgccgtttgtgatggcttccatgtcggcagaatgcttaatgaattacaacagtactgcgatgagtggcaggg
cggggcgtaaAATGGCGTTAATAAATAAGGAGGTAAGGTAATATGGCGAGCTATCCGTGTCACCAGCATG
CATCTGCTTTCGATCAGGCAGCGCGCAGCCGTGGTCATTCTAATCGTCGTACCGCACTGCGTCCGCG
TCGTCAGCAGGAGGCCACTGAGGTTCGTCTGGAGCAAAAGATGCCGACCCTGTTACGCGTATACATT
GATGGGCCGCATGGTATGGGTAAAACCACCACGACCCAATTACTGGTTGCGCTGGGCAGCCGTGAT
GATATTGTTTATGTGCCTGAACCGATGACGTATTGGCAGGTGCTGGGCGCGAGTGAAACTATTGCTA
ATATCTATACGACCCAGCATCGTCTGGACCAAGGGGAAATCAGCGCCGGTGATGCAGCCGTAGTGA
TGACCAGTGCGCAAATCACGATGGGTATGCCTTACGCAGTAACCGATGCGGTTCTGGCGCCGCATAT
TGGTGGTGAAGCCGGCAGTAGCCATGCGCCGCCGCCTGCCCTGACCCTGATTTTTGATCGTCACCCG
ATTGCGGCTCTGCTGTGCTATCCTGCTGCACGTTATCTGATGGGTTCTATGACCCCACAGGCCGTCCT
GGCATTCGTTGCACTGATTCCGCCTACTCTGCCTGGGACCAATATCGTGCTGGGGGCGCTGCCAGAA
GATCGTCATATCGACCGTCTGGCGAAACGTCAACGTCCTGGTGAACGCCTGGATCTGGCGATGCTGG
CAGCGATTCGTCGTGTATATGGCCTGCTGGCGAACACTGTCCGTTACCTGCAAGGCGGTGGCAGTTG
GCGTGAAGATTGGGGTCAACTGAGCGGTACGGCAGTTCCTCCGCAGGGTGCGGAACCTCAGTCTAA
CGCAGGTCCGCGTCCGCACATTGGTGATACCCTGTTCACCCTGTTCCGTGCGCCGGAGCTGCTGGCA
CCAAATGGGGATCTGTACAATGTTTTCGCGTGGGCGCTGGATGTTCTGGCTAAGCGTCTGCGCCCGA
TGCATGTTTTTATTCTGGATTATGATCAAAGCCCAGCAGGCTGTCGTGATGCGCTGCTTCAACTGACT
AGCGGCATGGTGCAAACGCATGTGACGACGCCTGGGAGTATCCCGACCATCTGTGATCTTGCCCGTA
CCTTCGCACGTGAAATGGGTGAAGCGAATGCCGAAGCTGCAGCAAAGGAGGCCGCAGCTAAAGCG
GCTGCAGCGAAAGCGGTGTCTAAAGGCGAAGCCGTTATTAAAGAATTCATGCGCTTCAAGGTTCAC
ATGGAGGGCTCGATGAATGGTCATGAGTTCGAGATTGAAGGGGAAGGTGAGGGCCGACCATATGAG
GGCACCCAAACTGCAAAACTGAAGGTTACTAAAGGTGGTCCGCTCCCGTTTAGTTGGGATATTCTGA
GCCCGCAGTTCATGTACGGCTCACGCGCTTTTATTAAGCATCCGGCGGACATACCGGACTACTATAA
ACAGTCCTTCCCGGAAGGGTTTAAATGGGAAAGAGTGATGAACTTTGAGGACGGAGGTGCGGTTAC
AGTGACTCAGGATACCAGTCTGGAGGATGGTACGCTGATCTATAAAGTAAAACTGCGTGGTACCAA
TTTTCCCCCAGATGGCCCCGTAATGCAGAAAAAAACCATGGGGTGGGAAGCATCGACCGAACGCCT
TTACCCGGAAGATGGCGTCTTGAAAGGAGACATCAAAATGGCTTTGCGCTTAAAAGATGGCGGCCG
TTATCTGGCGGATTTTAAAACGACCTACAAAGCCAAGAAACCTGTCCAAATGCCTGGTGCCTACAAC
GTGGATCGTAAACTAGACATCACGTCCCATAACGAAGATTATACAGTGGTCGAACAGTATGAACGG
AGCGAAGGCCGTCACAGCACGGGGGGAATGGACGAATTATATAAGTAACATTACTCGCATCCATTC
TCAGGCTctcggtaccaaattccagaaaagaggcctcccgaaaggggggccttttttcgttttggtccTACTGGCGCGCCTTTACgGCTAG
CTCAGTCCTAGGTAcTATGCTAGCaAGgTAGACTGTCGCCGGATGTGTATCCGACCTGACGATGGCCC
AAAAGGGCCGAAACAGTCCTCTACAAATAATTTTGTTTAATACTtcaTGGACgaaagtactagATGAATGAA
ACCGATCCTGATCTGGAACTGCTGAAACGTATTGGTAATAATGATGCACAGGCCGTTAAAGAAATG
GTTACCCGTAAACTGCCTCGTCTGCTGGCACTGGCAAGTCGCCTGCTGGGTGATGCAGATGAAGCAC
GTGATATTGCACAAGAAAGTTTTCTGCGCATTTGGAAACAGGCAGCAAGCTGGCGTAGCGAACAGG
CACGTTTTGATACCTGGCTGCATCGTGTTGCACTGAATCTGTGTTATGATCGTCTGCGTCGTCGTAAA
GAACATGTGCCGGTTGATAGCGAACATGCCTGTGAAGCATGCCTGAGCTACGAAACCGAAATCCTG
ACCGTTGAATATGGTCTGCTGCCGATCGGCAAAATCGTAGAAAAGCGTATCGAATGTACGGTTTACT
CTGTCGATAACAACGGTAACATCTACACCCAGCCGGTAGCGCAGTGGCACGACCGTGGCGAACAAG
AAGTGTTCGAGTACTGCCTGGAGGATGGCTCTCTGATCCGCGCTACTAAAGACCACAAATTTATGAC
CGTGGACGGTCAAATGCTGCCGATCGATGAAATCTTTGAGCGCGAACTGGACCTGATGCGCGTGGA
CAACCTGCCGAACATCAAAATTGCTACCCGCAAGTATCTGGGTAAGCAGAACGTCTATGACATTGGT
GTGGAGCGCGACCACAATTTCGCTCTGAAAAACGGAGGATCTGGTGGAAGTGGTGGTTCTGGAGGTc
gttttccgaatattaccaacttatgcccgtttggtgaggtgttcaacgcgacccgctttgccagcgtatacgcgtggaatcgtaaacgtatctcgaactgcgtagcggatt
actccgtgctttacaactcagcttccttctccacctttaaatgttatggtgtttcaccgaccaagttaaacgatctgtgctttacgaacgtctatgccgattcatttgtgatcag
aggtgatgaggttcgtcaaattgcgcctggacagacaggcaaaattgcagactataactacaaacttcccgacgattttacgggctgtgttattgcgtggaattcgaaca
acctggatagtaaggttggagggaattataactatctgtaccgcctgtttcgtaaatctaacctgaaacctttcgaacgcgacatatcaactgaaatctatcaggcaggta
gcactccctgtaacggtgtcgagggatttaactgctattttcctctgcagagttatggctttcagcctacgaatggagtaggctatcaaccgtaccgggtggtggttcttag
tttcgagctgctgcatgcaccagccacagtatgtggccccaaaaagtcaacgaatctttaaCTCGGTACCAAATTCCAGAAAAGAGACGC
TTTCGAGCGTCTTTTTTCGTTTTGGTCCcgcttactagtagcggccgctgcagtccggcaaaaaagggcaaggtgtcaccaccctgcccttt
ttctttaaaaccgaaaagattacttcgcgttatgcaggcttcctcgctcactgactcgctgcgctcggtcgttcggctgcggcgagcggtatcagctcactcaaaggcgg
taattcatgaccaaaatcccttaacgtgagttttcgttccactgagcgtcagaccccgtagaaaagatcaaaggatcttcttgagatcctttttttctgcgcgtaatctgctgc
ttgcaaacaaaaaaaccaccgctaccaacggtggtttgtttgccggatcaagagctaccaactctttttccgaaggtaactggcttcagcagagcgcagataccaaata
ctgtccttctagtgtagccgtagttaggccaccacttcaagaactctgtagcaccgcctacatacctcgctctgctaatcctgttaccagtggctgctgccagtggcgata
agtcgtgtcttaccgggttggactcaagacgatagttaccggataaggcgcagcggtcgggctgaacggggggttcgtgcacacagcccagcttggagcgaacga
cctacaccgaactgagatacctacagcgtgagctatgagaaagcgccacgcttcccgaagggagaaaggcggacaggtatccggtaagcggcagggtcggaac
aggagagcgcacgagggagcttccagggggaaacgcctggtatctttatagtcctgtcgggtttcgccacctctgacttgagcgtcgatttttgtgatgctcgtcaggg
gggcggagcctatggaaaaacgccagcaacgcggcctttttacggttcctggccttttgctggccttttgctcacatgttctttcctgcgttatcccctgattctgtggataa
ccgt (SEQ ID NO: 79)

TABLE 8
Protein modifying enzyme and peptide amino acid sequences

| | | SEQ ID |
|---|---|---|
| Name | Sequence | NO |
| EpiA | EAVKEKNDLFNLDVKVNAKESNDSGAEPRIASKFICTPGCAKTGSFNSYCC | 173 |
| EpiD | MHGKLLICATASINVININHYIVELKQHFDEVNILFSPSSKNFINTDVLKLFCDNLYDEIKD | 174 |
| | PLLNHINIVENHEYILVLPASANTINKIANGICDNLLTTVCLTGYQKLFIFPNMNIRMWGNP | |
| | FLQKNIDLLKSNDVKVYSPDMNKSFEISSGRYKNNITMPNIENVLNFVLNNEKRPLD | |
| LasA | MDKRVRYEKPSLVKEGTFRKTTAGLRRLFADQLVGRRNI | 175 |
| LasF | MSIELTPSLADLVDPLPGHALRAAATLRLADLIAAGADTAPALAAAARIDADAIARLMRYLC | 176 |
| | SRGIFQAHEGRYALTEFSELLLDEDPSGLRKTLDQDSYGDRFDRAVAELVDVVRSGEPSYPR | |
| | LYGSTVYDDLAADPALGEVFADVRGLHSAGYGEDVAAVAGWSSCLRVVDLGGGTGSVLLAVL | |
| | ERHPSLSGAVLDLPYVAPQAKKALQASAFAQRCEFIKGSFFDPLPPADRYLLCNVLFNWDDA | |
| | QAGAILARCAQAGPVAGVVVAERLIDPDAEVELVAAQDLRLLAVCGGRQRGTAEFEALGAAH | |
| | GLALTSVTLTASGMSLLRFDVCRAGSAGGEVVEKS | |
| TruE | MNKKNILPQLGQPVIRLTAGQLSSQLAELSEEALGGVDASTLPVPTLCSYDGVDASTVPTLC | 177 |
| | SYDD | |
| TruE* | MNKKNILPQLGQPVIRLTAGQLSSQLAELSEEALGGVDASTVPTLCSYDD | 178 |
| LynD | MQSTPLLQIQPHFHVEVIEPKQVYLLGEQANHALTGQLYCQILPLLNGQYTLEQIVEKLDGE | 179 |
| | VPPEYIDYVLERLAEKGYLTEAAPELSSEVAAFWSELGIAPPVAAEALRQPVTLTPVGNISE | |
| | VTVAALTTALRDIGISVQTPTEAGSPTALNVVLTDDYLQPELAKINKQALESQQTWLLVKPV | |
| | GSVLWLGPVFVPGKTGCWDCLAHRLRGNREVEASVLRQKQAQQQRNGQSGSVIGCLPTARAT | |
| | LPSTLQTGLQFAATEIAKWIVKYHVNATAPGTVFFPTLDGKIITLNHSILDLKSHILIKRSQ | |
| | CPTCGDPKILQHRGFEPLKLESRPKQFTSDGGHRGTTPEQTVQKYQHLISPVTGVVTELVRI | |
| | TDPANPLVHTYRAGHSFGSATSLRGLRNTLKHKSSGKGKTDSQSKASGLCEAVERYSGIFQG | |
| | DEPRKRATLAELGDLAIHPEQCLCFSDGQYANRETLNEQATVAHDWIPQRFDASQAIEWTPV | |
| | WSLTEQTHKYLPTALCYYHYPLPPEHRFARGDSNGNAAGNTLEEAILQGFMELVERDGVALW | |
| | WYNRLRRPAVDLGSFNEPYFVQLQQFYRENDRDLWVLDLTADLGIPAFAGVSNRKTGSSERL | |
| | ILGFGAHLDPTIAILRAVTEVNQIGLELDKVPDENLKSDATDWLITEKLADHPYLLPDTTQP | |
| | LKTAQDYPKRWSDDIYTDVMTCVNIAQQAGLETLVIDQTRPDIGLNVVKVTVPGMRHFWSRF | |
| | GEGRLYDVPVKLGWLDEPLTEAQMNPTPMPF | |
| PaaA | MSLTNVKPLIKESHHIILADDGDICIGEIPGVSQVINDPPSWVRPALAKMDGKRTVPRIFKE | 180 |
| | LVSEGVQIESEHLEGLVAGLAERKLLQDNSFFSKVLSGEEVERYNRQILQFSLIDADNQHPF | |
| | VYQERLKQSKVAIFGMGGWGTWCALQLAMSGIGTLRLIDGDDVELSNINRQVLYRTDDVGKN | |
| | KVDAAKDTILAYNENVHVETFFEFASPDRARLEELVGDSTFIILAWAALGYYRKDTAEEIIH | |
| | SIAKDKAIPVIELGGDPLEISVGPIYLNDGVHSGFDEVKNSVKDKYYDSNSDIRKFQEARLK | |
| | HSFIDGDRKVNAWQSAPSLSIMAGIVTDQVVKTITGYDKPHLVGKKFILSLQDFRSREEEIF | |
| | K | |
| PaaP | MIKFSTLSQRISAITEENAMYTKGQVIVLS | 181 |
| PadeA | MKKQYSKPSLEVLDVHQTMAGPGTSTPDAFQPDPDEDVHYDS | 182 |
| PadeK | MTERAAVRTDHYKAFGFRIESDFVLPELPPAGEREPLDNITVRRTDLQPLWNSSIHFYGNFA | 183 |
| | ILDHGRTVMFRVPGAAIYAVQDASSILVSPFDQAEENWVRLFILGTCIGIILLQRKIMPLHG | |
| | SAVAIDGKAYAIIGESGAGKSTLALHLVSKGYPLLSDDVIPVVMTQGSPWVVPSYPQQKLWV | |
| | DTLKHMGMDNANYTPLYERKTKFAVPVGSNFHEEPLPLASIFELVPWDAATHIAPIQGMERF | |
| | RVLFHHTYRNFLVQPLGLMEWHFKTLSSFVHQIGMYRLHRPMVGFSTLDLTSHILNITRQGE | |
| | NDQ | |
| PalA | MKDLLKELMYEVDLEEMENLQGSGYSAAQCAWMALSCVNYIPGVGFGCGGYSACELYKRYC | 184 |
| PalS | MGNLRDFYQLMKDNYADSNLFKDLNLIHNISNDIQIGINCDFSEMLGELVGNYDSLNYPSIT | 185 |
| | CGILTYNEERCIKRCLESVVNEFDEIIVLDSVSEDNTVKIIKENFNDVKVYVEPWKNDFSFH | |
| | RNKIINLATCDWIYFIDADNYYDSKNKGKAMRIAKVMDFLKIEGVVSPTVIEHDNSMSRDTR | |
| | KMFRLKDNILFSGKVHEEPVYANGEIPRNIIVDINVFHDGYNPKIINMMEKNERNITLTKEM | |
| | MKIEPNNPKWLYFYSRELYQTQRDIALVQSVLFKALELYENSSYTRYYVDTIALLCRVLFES | |
| | KNYQKLTECLNILENNTLNCSDIDYYNSALLFYNLLLRIKKISSTLKENIDMYERDYHSFIN | |
| | PSHDHIKILILNMLLLLGDYQDAFKVYKEIKSIEIKDEFLVNVNKFKDNLLSFIDSINKI | |
| PlpA2 | MSIESAKAFYQRMTDDASFRTPFEAELSKEERQQLIKDSGYDFTAEEWQQAMTEIQAARSNE | 186 |
| | ELNEEELEAIAGGAVAAMYGVVFPWDNEFPWPRWGG | |
| PlpX | MTKKYRRVSYAVWEITLKCNLACSHCGSRAGQARTKELSTEEAFNLVRQLADVGIKEVTLIG | 187 |
| | GEAFMRSDWLEIAKAVTEAGMICGMTTGGFGVSLETARKMKEAGIKTVSVSIDGGIPETHDR | |
| | QRGKKGAWHSAFRTMSHLKEVGIYFGCNTQINRLSASEFPIIYERIRDAGARAWQIQLTVPM | |
| | GNAADNADMLLQPYELLDIYPMLARVAKRAKQEGVRIQAGNNIGYYGPYERLLRGSDEWTFW | |
| | QGCGAGLNTLGIEADGKIKGCPSLPTAAYTGGNIRDRPLREIVEQTEELKFNLKAGTEQGTD | |
| | HMWGFCKTCEFAELCRGGCSWTAHVFFDRRGNNPYCHHRALKQAQKDIRERFYLKVKAKGNP | |
| | FDNGEFVIIEEPFNAPLPENDLLHFNSDHIQWPENWQNSESAYALAK | |
| PlpY | MNSNQIPNKVATAAQKSDDSSSVLPRQGWQDKQAFIKALIKAKQSLEIAEISNFLT | 188 |
| TgnA* | MYRPYIAKYVEEQTLQNSTNLVYDDITQISFINKEKNVKKINLGPDTTIVTETIENADPDEY | 189 |
| | FL | |
| TgnB | MKTILIITNTLDLTVDYIINRYNHTAKFFRLNTDRFFDYDINITNSGTSIRNRKSNLIINIQ | 190 |
| | EIHSLYYRKITLPNLDGYESKYWTLMQREMMSIVEGIAETAGNFALTRPSVLRKADNKIVQM | |
| | KLAEEIGFILPQSLITNSNQAAASFCNKNNTSIVKPLSTGRILGKNKIGIIQTNLVETHENI | |
| | QGLELSPAYFQDYIPKDTEIRLTIVGNKLFGANIKSTNQVDWRKNDALLEYKPANIPDKIAK | |
| | MCLEMMEKLEINFAAFDFIIRNGDYIFLELNANGQWLWLEDILKFDISNTIINYLLGEPI | |
| ThcoA | MRKKEWQTPELEVLDVRLTAAGPGKAKPDAVQPDEDEIVHYS | 191 |
| ThcoK | MTRTNTGYRYRAFGLRIDSDIPLPELGDGTRPDGDADLTVVRCGEAEPEWAEGGGGGRLYAA | 192 |
| | EGIVSFRVPQTAAFRITNGNRIEVHAYSGADEDRIRLYVLGTCMGALLLQRRILPLHGSVVA | |
| | RDGRAYAIVGESGAGKSTMSAALLERGFRLVTDDVAAIVFDERGTPLVMPAYPQQKLWQDSL | |
| | DRLQIAGSGLRPLFERETKYAVPADGAFWPEPVPLVHIYELVHSDGQTPELQPIAKLERCYT | |
| | LYRHTFRRSLIVPSGLSAWHFETAVKLAEKTGMYRLMRPAKVFAARESARLIETHADGEVSR | |

*TruE and TgnA peptides used in this study were truncated relative to the wild-type peptides.

TABLE 9
Genetic parts

| | | |
|---|---|---|
| | | SEQ |
| Name | Sequence | ID NO |

Promoters

| | | |
|---|---|---|
| P_{CymRC} | AACAAACAGACAATCTGGTCTGTTTGTATTATGGAAAATTTTTCTGTATAATAGAT | 193 |
| | TCAACAAACAGACAATCTGGTCTGTTTGTATTAT | |
| P_{LacI} | GCGGCGCGCCATCGAATGGCGCAAAACCTTTCGCGGTATGGCATGATAGCGCCC | 194 |
| P_{LacIQ} | GCGGCGCGCCATCGAATGGTGCAAAACCTTTCGCGGTATGGCATGATAGCGCCC | 195 |
| PT5_{LacO} | AATCATAAAAAATTTATTTGCTTTGTGAGCGGATAACAATTATAATAGATTCAATT | 196 |
| | GTGAGCGGATAACAATT | |

Ribosome Binding Sites

| | | |
|---|---|---|
| RBS_{EpiD} | ACTGAACTATAAGGTAGGTATATT | 197 |
| RBS_{LacI} | GGAAGAGAGTCAATTCAGGGTGGTGAAT | 198 |
| RBS_{LasF} | AGAGCCATCAGATTTAAGGAACATAAAAA | 199 |
| RBS_{LynD} | CTAAATTCCCCCGAGGTCAATA | 200 |
| RBS_{PaaA} | AGATCATTTCCAATAAGGGGGACACT | 201 |
| RBS_{PadeK} | AGACACCGAAACCTAAGGAGGGATAT | 202 |
| RBS_{PalS} | AGACCAAACAATTAGGAGGACAAAT | 203 |
| RBS_{peptide} | ACCCAACACCACCAGCAAGCCTAAGGAGGAGAAAT | 204 |
| RBS_{Plp}x^{a} | AGAGCCACCATTTATAAGGAGAACCTACCG | 205 |
| RBS_{Plp}Y^{a} | ATATAAAGTTAAGGAGTTGCAC | 206 |
| RBS_{TgnB} | AGAAATATTACAACGAGGTAAAGGC | 207 |
| RBS_{TheoK} | AGAGCATTCCATAAGGAGAAATTTT | 208 |

Terminators

| | | |
|---|---|---|
| B0062 | CAGATAAAAAAAATCCTTAGCTTTCGCTAAGGATGATTTCT | 209 |
| ECK120029600 | TTCAGCCAAAAAACTTAAGACCGCCGGTCTTGTCCACTACCTTGCAGTAATGCGGT | 210 |
| | GGACAGGATCGGCGGTTTTCTTTTCTCTTCTCAA | |
| AraC Terminator | TTGGTAACGAATCAGACAATTGACGGCTCGAGGGAGTAGCATAGGGTTTGCAGAAT | 211 |
| w/ 2 SNPs | CCCTGCTTCGTCCATTTGACAGGCACATTATGCATCGATGATAAGCTGTCAAACAT | |
| | GAGCA | |
| His operon | TCCGGCAAAAAAGGGCAAGGTGTCACCACCCTGCCCTTTTTCTTTAAAACCGAAAA | 212 |
| terminator | GA | |
| L3S3P21 | CCAATTATTGAAGGCCTCCCTAACGGGGGGCCTTTTTTTGTTTCTGGTCTCCC | 213 |
| L3S3P41^{b} | AAAAAAAAAAAACACCCTAACGGGTGTTTTTTTTTTTTTGGTGTCCC | 214 |
| IOT | TTGGTAACGAATCAGACAATTGACGGCTCGAGGGAGTAGCATAGGGTTTGCAGAAT | 215 |
| | CCCTGCTTCGTCCATTTGACAGGCACATTATGCATCGATGATAAGCTGTCAAACAT | |
| | GAGCAGATCCTCTACGCCGGACGCATCGTGGCCGGCATCACCGGCGCCACAGGTGC | |
| | GGTTGCTGGCGCCTATATCGCCGACATCACCGATGGGGAAGATCGGGCTCGCCACT | |
| | TCGGGCTCATGAGCAAATATTTTATCTG | |

Ribozymes

| | | |
|---|---|---|
| RiboJ53 | AGCGGTCAACGCATGTGCTTTGCGTTCTGATGAGACAGTGATGTCGAAACCGCCTC | 216 |
| | TACAAATAATTTTGTTTAA | |

Linkers/Tags

| | | |
|---|---|---|
| N-terminal sumo | ATGTCATATTACCACCATCACCATCATCACGGGTCCCTGCAG | 217 |
| affinity tag | | |
| (ATag-2) | | |
| N-terminal sumo | CATCACCATCACCACCATGGATATGATATTAGCACAGGT | 218 |
| linker v1 (Link- | | |
| 1) | | |
| N-terminal sumo | TGCATGTCATATTACGACTCCATTCCCACAAGCGAGAACTTGTACTTTCAAGGGTG | 219 |
| linker v2 (Link- | C | |
| 2) | | |

Genes Miscellaneous

| | | |
|---|---|---|
| Small Ubiquitin- | GACTCAGAAGTCAATCAAGAAGCTAAGCCAGAGGTCAAGCCAGAAGTCAAGCCTGA | 220 |
| like Modifier | GACTCACATCAATTTAAAGGTGTCCGATGGATCTTCAGAGATCTTCTTCAAGATCA | |
| (SUMO) | AAAAGACCACTCCTTTAAGAAGGCTGATGGAAGCGTTCGCTAAAAGACAGGGTAAG | |
| | GAAATGGACTCCTTAAGATTCTTGTACGACGGTATTAGAATTCAAGCTGATCAGGC | |
| | CCCTGAAGATTTGGACATGGAGGATAACGATATTATTGAGGCTCACCGCGAACAGA | |
| | TTGGAGGT | |
| lacI | ATGAAACCAGTAACGTTATACGATGTCGCAGAGTATGCCGGTGTCTCTTATCAGAC | 221 |
| | CGTTTCCCGCGTGGTGAACCAGGCCAGCCACGTTTCTGCGAAAACGCGGGAAAAAG | |
| | TGGAAGCGGCGATGGCGGAGCTGAATTACATTCCCAACCGCGTGGCACAACAACTG | |
| | GCGGGCAAACAGTCGTTGCTTATTGGCGTTGCCACCTCCAGTCTGGCCCTGCACGC | |
| | GCCGTCGCAAATTGTCGCGGCGATTAAATCTCGCGCCGATCAACTGGGTGCCAGCG | |
| | TGGTGGTGTCGATGGTAGAACGAAGCGGCGTCGAAGCCTGTAAAGCGGCGGTGCAC | |
| | AATCTTCTCGCGCAACGCGTCAGTGGGCTGATCATTAACTATCCGCTGGATGACCA | |
| | GGATGCCATTGCTGTGGAAGCTGCCTGCACTAATGTTCCGGCGTTATTTCTTGATG | |
| | TCTCTGACCAGACACCCATCAACAGTATTATTTTCTCCCATGAGGACGGTACGCGA | |
| | CTGGGCGTGGAGCATCTGGTCGCATTGGGTCACCAGCAAATCGCGCTGTTAGCGGG | |
| | CCCATTAAGTTCTGTCTCGGCGCGTCTGCGTCTGGCTGGCTGGCATAAATATCTCA | |
| | CTCGCAATCAAATTCAGCCGATAGCGGAACGGGAAGGCGACTGGAGTGCCATGTCC | |
| | GGTTTTCAACAAACCATGCAAATGCTGAATGAGGGCATCGTTCCCACTGCGATGCT | |
| | GGTTGCCAACGATCAGATGGCGCTGGGCGCAATGCGCGCCATTACCGAGTCCGGGC | |
| | TGCGCGTTGGTGCGGATATCTCGGTAGTGGGATACGACGATACCGAAGATAGCTCA | |
| | TGTTATATCCCGCCGTTAACCACCATCAAACAGGATTTTCGCCTGCTGGGGCAAAC | |
| | CAGCGTGGACCGCTTGCTGCAACTCTCTCAGGGCCAGGCGGTGAAGGGCAATCAGC | |
| | TGTTGCCAGTCTCACTGGTGAAAAGAAAAACCACCCTGGCGCCCAATACGCAAACC | |
| | GCCTCTCCCCGCGCGTTGGCCGATTCATTAATGCAGCTGGCACGACAGGTTTCCCG | |
| | ACTGGAAAGCGGGCAGTGATAA | |
| cymR | ATGAGCCCGAAACGTCGTACCCAGGCAGAACGTGCAATGGAAACCCAGGGTAAACT | 222 |
| | GATTGCAGCAGCACTGGGTGTTCTGCGTGAAAAAGGTTATGCAGGTTTTCGTATTG | |
| | CAGATGTTCCGGGTGCAGCCGGTGTTAGCCGTGGTGCACAGAGCCATCATTTTCCG | |
| | ACCAAACTGGAACTGCTGCTGGCAACCTTTGAATGGCTGTATGAGCAGATTACCGA | |
| | ACGTAGCCGTGCACGTCTGGCAAAACTGAAACCGGAAGATGATGTTATTCAGCAGA | |
| | TGCTGGATGATGCAGCAGATTTTTTTCTGGATGATGATTTTAGCATCGGCCTGGAT | |
| | CTGATTGTTGCAGCAGATCGTGATCCGGCACTGCGTGAAGGTATTCTGCGTACCGT | |
| | TGAACGTAATCGTTTTGTTGTTGAAGATATGTGGCTGGGTGTGCTGGTGAGCCGTG | |
| | GTCTGAGCCGTGATGATGCCGAAGATATTCTGTGGCTGATTTTTAACAGCGTTCGT | |
| | GGTCTGACAGTTCGTAGCCTGTGGCAGAAAGATAAAGAACGTTTTGAACGTGTGCG | |
| | TAATAGCACCCTGGAAATTGCACGTGAACGTTATGCAAAATTCAAACGTTGATAA | |

Modifying Enzymes

| | | |
|---|---|---|
| epiD | ATGCACGGTAAACTGCTGATCTGCGCAACTGCTTCGATCAACGTCATCAATATCAA | 223 |
| | CCATTATATTGTGGAGCTGAAACAGCACTTCGATGAGGTGAATATCCTGTTTTCAC | |
| | CTTCCTCGAAGAACTTTATCAACACCGATGTCCTGAAGCTGTTTTGCGATAATCTG | |
| | TATGACGAGATCAAAGATCCGCTGCTGAACCACATCAACATAGTGGAGAACCACGA | |
| | GTATATCTTGGTGCTGCCTGCCAGTGCCAATACGATCAACAAAATCGCGAACGGTA | |
| | TATGCGATAACCTCTTGACGACCGTATGCTTAACCGGGTACCAGAAACTGTTTATC | |
| | TTTCCGAATATGAACATCCGCATGTGGGGAAATCCGTTCTTACAGAAAAATATTGA | |
| | CCTGCTTAAAAGCAACGACGTGAAGGTGTATTCCCCCGACATGAACAAATCTTTTG | |
| | AGATAAGCTCAGGCCGCTACAAAAATAACATCACGATGCCGAATATCGAAAACGTG | |
| | CTGAATTTTGTCCTGAACAATGAGAAACGCCCGCTGGATTAATAA | |
| lasF | ATGTCTATCGAACTGACGCCTAGTTTGGCCGATCTGGTCGATCCACTTCCAGGTCA | 224 |
| | CGCACTGCGCGCTGCGGCGACATTACGTCTGGCAGATCTGATTGCGGCTGGTGCAG | |
| | ATACTGCACCGGCATTAGCAGCGGCGGCACGCATTGATGCTGACGCGATCGCGCGT | |
| | CTTATGCGGTATCTGTGCAGTCGCGGGATTTTTCAAGCACATGAAGGCCGGTACGC | |
| | GTTGACTGAATTTAGCGAATTGCTGCTGGATGAAGATCCATCTGGCCTGCGTAAAA | |
| | CCTTAGATCAGGATAGCTATGGGGATCGTTTCGACCGCGCGGTTGCGGAACTGGTG | |
| | GACGTTGTACGGTCCGGTGAACCTTCTTATCCTCGCCTTTACGGCTCGACGGTTTA | |
| | TGATGACCTGGCAGCCGATCCTGCCCTCGGCGAGGTGTTCGCGGATGTTCGTGGCT | |
| | TGCACTCCGCAGGGTATGGGGAAGATGTCGCGGCAGTGGCGGGTTGGTCCTCATGC | |
| | CTGCGCGTTGTCGATCTGGGTGGAGGGACTGGCTCCGTCCTGCTTGCTGTGTTAGA | |
| | GCGTCACCCGTCCCTGTCAGGCGCAGTACTGGATCTGCCATACGTCGCCCCGCAGG | |
| | CAAAGAAAGCTCTGCAGGCCTCAGCGTTTGCCCAACGTTGTGAATTTATCAAAGGG | |
| | AGCTTCTTCGATCCGTTACCTCCGGCAGACCGTTACCTGTTGTGTAACGTGCTGTT | |
| | CAACTGGGATGACGCGCAAGCAGGCGCTATTTTGGCACGCTGTGCGCAGGCGGGCC | |
| | CTGTGGCCGGAGTAGTGGTAGCCGAACGTTTGATCGATCCGGATGCGGAAGTGGAA | |
| | CTCGTAGCAGCTCAAGATCTGCGTCTGTTGGCTGTTTGCGGCGGTCGGCAGCGTGG | |
| | CACCGCTGAATTCGAAGCGCTTGGGGCAGCCCATGGCCTGGCGTTAACCAGCGTTA | |
| | CCCTCACGGCATCTGGTATGAGCCTGCTCCGTTTCGATGTGTGTCGTGCCGGGAGT | |
| | GCTGGCGGGGAAGTTGTGGAAAAATCTTAATAA | |
| lynD | ATGCAATCTACACCATTACTGCAAATACAACCACATTTCCATGTAGAGGTCATTGA | 225 |
| | ACCAAAGCAAGTCTACTTGTTGGGTGAACAAGCTAATCATGCATTGACAGGCCAAT | |
| | TATACTGCCAAATTTTGCCATTGTTAAACGGACAATACACATTGGAACAAATCGTT | |
| | GAAAAACTAGACGGAGAAGTACCACCTGAATACATTGATTATGTGCTGGAGAGACT | |
| | AGCTGAGAAGGGCTATCTGACTGAAGCAGCACCTGAATTATCTAGTGAAGTGGCCG | |
| | CTTTCTGGTCTGAGCTGGGGATTGCACCTCCTGTCGCGGCCGAAGCATTACGTCAA | |
| | CCTGTGACTTTAACACCTGTTGGAAACATCAGCGAAGTAACAGTAGCAGCCTTAAC | |
| | CACAGCCCTACGTGATATCGGTATTTCCGTTCAAACACCTACAGAAGCTGGATCGC | |
| | CAACTGCATTGAACGTTGTACTTACCGATGATTATCTCCAACCAGAACTCGCTAAG | |
| | ATCAATAAGCAAGCCTTAGAAAGTCAACAAACTTGGCTACTTGTCAAACCAGTTGG | |
| | CTCCGTGTTATGGTTGGGTCCGGTATTCGTGCCAGGAAAAACAGGTTGCTGGGATT | |
| | GTTTGGCTCACAGATTAAGGGGGAATAGAGAGGTAGAGGCCTCTGTATTGAGACAA | |
| | AAACAAGCTCAACAACAACGTAATGGACAAAGCGGGTCTGTAATAGGATGCCTTCC | |
| | CACGGCTAGAGCGACACTGCCCTCAACACTCCAAACTGGGCTGCAGTTCGCTGCTA | |
| | CCGAAATTGCTAAATGGATAGTTAAGTATCATGTTAATGCCACAGCGCCTGGCACC | |
| | GTATTCTTCCCTACATTGGATGGTAAGATAATTACGCTAAATCACTCCATACTGGA | |
| | TTTGAAGTCACATATTCTGATCAAGCGTTCTCAATGTCCCACCTGTGGTGACCCAA | |
| | AAATCTTACAGCACCGTGGTTTCGAACCTTTAAAACTTGAGTCAAGGCCTAAACAG | |
| | TTCACCTCAGACGGCGGACATCGTGGTACTACCCCTGAACAAACTGTCCAGAAATA | |
| | TCAACATTTAATCTCGCCTGTTACCGGTGTAGTTACTGAATTGGTCAGGATAACTG | |
| | ATCCGGCCAATCCACTAGTTCACACATATAGAGCTGGTCATAGCTTCGGGAGCGCT | |
| | ACATCGCTGAGAGGGCTGCGTAATACCTTAAAGCATAAGAGTTCAGGTAAGGGTAA | |
| | GACTGATTCTCAAAGTAAAGCCTCGGGCCTGTGTGAGGCGGTAGAACGTTACTCAG | |
| | GAATCTTTCAAGGTGACGAACCGAGAAAACGCGCCACATTGGCTGAATTGGGAGAT | |
| | TTGGCAATTCACCCTGAGCAATGCTTGTGTTTTTCCGACGGTCAGTACGCTAATAG | |
| | AGAAACTTTAAACGAACAGGCAACGGTGGCACATGATTGGATACCTCAACGTTTTG | |
| | ATGCATCACAAGCTATTGAATGGACTCCAGTCTGGTCCCTAACTGAACAGACCCAT | |
| | AAATATTTGCCCACCGCATTGTGTTACTACCATTATCCTCTACCCCCAGAACACAG | |
| | ATTCGCACGTGGAGATTCGAATGGTAATGCTGCCGGAAATACGTTGGAAGAGGCTA | |
| | TACTCCAAGGCTTCATGGAATTAGTCGAGAGAGATGGTGTGGCTTTATGGTGGTAT | |
| | AACAGGCTACGCAGACCCGCTGTAGACTTAGGCTCATTTAACGAGCCATACTTCGT | |
| | TCAGTTGCAACAATTCTACAGAGAAAACGATAGAGATTTGTGGGTTTTGGACTTGA | |
| | CAGCTGATTTAGGTATCCCGGCTTTCGCGGGCGTTTCTAATAGAAAAACTGGTAGT | |
| | TCGGAGAGGTTGATATTAGGATTCGGTGCACACCTCGATCCTACTATTGCAATTCT | |
| | GAGAGCAGTTACAGAAGTTAACCAGATTGGCCTTGAATTAGATAAAGTTCCAGACG | |
| | AGAACCTTAAGAGCGACGCAACAGATTGGCTAATTACTGAAAAATTAGCTGACCAC | |
| | CCTTATTTGTTACCAGATACAACTCAACCTCTAAAAACTGCTCAAGATTATCCTAA | |
| | AAGGTGGTCTGACGATATATACACGGACGTAATGACTTGCGTTAATATTGCTCAAC | |
| | AAGCAGGACTTGAAACTCTAGTTATTGATCAAACACGTCCGGACATTGGTTTGAAT | |
| | GTTGTTAAGGTGACAGTCCCGGGGATGAGGCACTTTTGGTCAAGATTTGGAGAGGG | |
| | GAGGCTTTATGACGTGCCCGTCAAATTAGGTTGGCTTGACGAACCTTTGACCGAAG | |
| | CGCAAATGAACCCCACGCCGATGCCTTTTTAATAA | |
| paaA | ATGAGCCTGACGAATGTCAAGCCGTTGATTAAAGAATCCCACCACATCATTTTAGC | 226 |
| | TGACGATGGTGACATTTGCATTGGGGAAATTCCGGGGGTGTCTCAGGTAATCAATG | |
| | ACCCGCCGTCGTGGGTTCGTCCTGCCCTGGCAAAGATGGATGGCAAGCGTACTGTC | |
| | CCCCGTATTTTCAAAGAACTGGTCAGTGAAGGCGTACAGATCGAATCCGAACATCT | |
| | GGAAGGCCTGGTAGCCGGGCTTGCCGAACGCAAACTTCTCCAGGATAACAGTTTCT | |
| | TTTCCAAGGTGTTAAGCGGTGAAGAAGTGGAGCGCTATAACCGCCAGATTCTGCAG | |
| | TTCAGCCTTATCGATGCGGATAACCAGCACCCTTTCGTTTACCAAGAGCGGCTGAA | |
| | ACAGTCTAAAGTCGCTATCTTCGGTATGGGTGGCTGGGGCACGTGGTGTGCATTGC | |
| | AGCTGGCCATGTCAGGCATTGGTACACTGCGGCTGATCGACGGCGATGATGTGGAA | |
| | CTGTCGAACATTAACCGCCAAGTTCTGTATCGCACGGATGATGTAGGTAAAAACAA | |
| | AGTTGATGCCGCCAAAGACACTATCCTGGCATACAACGAAAACGTGCATGTTGAAA | |
| | CCTTCTTTGAATTCGCCAGCCCGGACCGTGCCCGGCTTGAAGAACTTGTGGGTGAT | |
| | TCTACCTTTATTATCCTGGCTTGGGCCGCGTTGGGTTACTACCGTAAAGATACGGC | |
| | AGAGGAAATTATCCATTCGATTGCGAAAGATAAAGCGATCCCTGTAATTGAACTCG | |
| | GCGGTGATCCTTTGGAAATCTCTGTCGGTCCTATTTACCTGAATGATGGCGTACAC | |
| | AGCGGCTTCGACGAGGTGAAAAATTCCGTTAAAGATAAATACTACGACAGCAACAG | |
| | CGATATCCGCAAATTTCAAGAGGCGCGGTTGAAACACAGCTTCATCGATGGCGATC | |
| | GTAAAGTGAACGCGTGGCAATCAGCGCCCAGCCTGAGTATTATGGCTGGTATCGTA | |
| | ACGGATCAGGTTGTGAAAACCATTACCGGGTACGACAAGCCACATCTCGTTGGCAA | |
| | GAAATTTATCTTGAGTCTGCAAGATTTCCGCAGCCGCGAGGAGGAGATCTTTAAAT | |
| | AATAA | |
| padeK | ATGACCGAACGTGCCGCAGTGCGTACCGACCATTATAAAGCCTTTGGGTTTAGAAT | 227 |
| | TGAAAGCGATTTCGTGCTCCCGGAACTTCCGCCCGCAGGCGAACGCGAACCGCTCG | |
| | ATAATATTACGGTTCGTCGTACCGACCTGCAGCCGCTCTGGAATTCTAGTATCCAT | |
| | TTTTACGGAAACTTTGCCATTCTGGATCACGGACGCACGGTTATGTTTCGAGTTCC | |
| | GGGTGCTGCTATCTATGCGGTACAGGATGCTAGCAGCATATTAGTGTCCCCATTCG | |
| | ATCAGGCAGAAGAAAACTGGGTACGTCTTTTTATTCTGGGTACCTGTATTGGGATC | |
| | ATCCTGCTGCAGCGTAAGATTATGCCGCTGCACGGTAGCGCCGTTGCCATTGATGG | |
| | CAAAGCCTACGCGATTATCGGCGAATCTGGTGCCGGCAAAAGCACTCTTGCACTGC | |
| | ATCTTGTCAGTAAGGGTTATCCATTGCTTTCGGATGATGTGATTCCGGTCGTTATG | |
| | ACCCAGGGCTCCCCCTGGGTGGTGCCGTCGTACCCGCAACAAAAACTTTGGGTGGA | |
| | CACTCTGAAGCACATGGGAATGGATAATGCAAACTATACGCCGCTGTACGAACGTA | |
| | AAACGAAGTTCGCGGTGCCCGTGGGCAGTAATTTCCACGAAGAACCGCTGCCGTTA | |
| | GCTAGCATTTTCGAGCTTGTCCCGTGGGATGCGGCAACGCACATTGCCCCGATCCA | |
| | AGGGATGGAACGCTTTCGTGTCCTGTTCCACCACACTTATCGGAACTTTCTGGTTC | |
| | AGCCGCTGGGTCTTATGGAATGGCATTTTAAAACTCTGAGCTCGTTCGTTCACCAA | |
| | ATTGGAATGTATCGTCTGCATAGACCTATGGTCGGATTCAGTACCTTAGATTTAAC | |
| | GTCGCACATTCTGAATATAACGCGTCAGGGAGAGAACGATCAATAATAA | |
| palS | ATGGGGAATTTGCGTGATTTCTACCAACTGATGAAAGATAACTATGCGGACTCTAA | 228 |
| | TCTGTTCAAGGATTTGAATCTGATCCACAATATCTCCAACGACATCCAAATTGGAA | |
| | TTAATTGCGATTTCTCTGAAATGCTGGGAGAACTGGTAGGTAATTACGATTCCCTG | |
| | AACTATCCGTCAATCACCTGTGGTATTCTGACGTATAATGAAGAACGCTGCATTAA | |
| | ACGTTGTCTGGAAAGTGTGGTGAACGAATTCGATGAGATTATTGTCTTGGATAGTG | |
| | TATCCGAGGACAATACCGTGAAAATTATCAAGGAGAATTTCAACGATGTCAAAGTC | |
| | TACGTCGAGCCATGGAAGAACGATTTTTCATTTCACCGCAACAAGATCATTAATCT | |
| | CGCAACGTGCGACTGGATCTACTTTATCGACGCGGATAATTATTATGATTCGAAGA | |
| | ACAAGGGTAAAGCCATGCGCATCGCTAAGGTTATGGATTTCTTGAAAATCGAAGGC | |
| | GTTGTGAGCCCAACGGTCATTGAGCATGACAATAGCATGAGCCGTGATACCCGTAA | |
| | GATGTTTCGTCTGAAAGATAACATTCTGTTTAGCGGTAAAGTTCATGAAGAACCGG | |
| | TGTATGCCAATGGTGAGATCCCCCGGAACATCATAGTAGACATCAACGTGTTTCAC | |
| | GACGGCTATAACCCAAAGATTATCAACATGATGGAAAAGAACGAGCGCAATATCAC | |
| | CCTGACTAAAGAGATGATGAAGATCGAACCGAACAATCCGAAATGGCTGTACTTCT | |
| | ATAGCCGCGAACTCTATCAGACGCAACGTGACATTGCCCTTGTGCAAAGTGTACTG | |
| | TTCAAGGCACTGGAACTGTATGAAAACAGTTCATATACGCGTTATTATGTTGACAC | |
| | CATTGCCTTACTGTGCCGAGTGCTGTTCGAATCTAAAAACTACCAGAAACTTACGG | |
| | AATGTCTGAACATCCTGGAGAACAATACGCTTAACTGTTCCGATATCGATTACTAT | |
| | AATTCAGCGCTGCTGTTCTACAACCTGTTACTGCGCATCAAGAAAATTAGCTCCAC | |
| | CCTGAAGGAGAACATTGATATGTACGAACGTGACTATCATAGCTTTATCAACCCCT | |
| | CGCATGATCACATTAAGATTCTGATATTAAATATGCTCCTGCTGCTCGGCGATTAC | |
| | CAGGATGCCTTTAAGGTTTACAAGGAGATCAAGTCCATTGAGATTAAAGATGAGTT | |
| | TCTGGTGAACGTGAACAAATTCAAAGACAATCTTCTGAGCTTCATTGACTCCATTA | |
| | ACAAAATTTAATAA | |
| plpX^{a} (Expressed | ATGACCAAAAAGTATCGGCGTGTATCCTACGCAGTGTGGGAAATCACCCTGAAATG | 229 |
| as plpXY) | CAATCTGGCATGCTCTCATTGTGGCAGCCGCGCCGGCCAAGCCCGTACGAAAGAGC | |
| | TGAGTACCGAAGAAGCGTTCAACCTGGTCCGCCAGCTGGCCGACGTGGGCATTAAG | |
| | GAAGTCACCCTGATCGGTGGTGAAGCCTTTATGCGTTCGGATTGGCTGGAAATCGC | |
| | GAAAGCCGTCACTGAAGCCGGCATGATCTGTGGCATGACCACAGGGGGCTTCGGGG | |
| | TCAGTCTGGAAACGGCGCGTAAAATGAAAGAAGCGGGCATTAAAACGGTGAGCGTT | |
| | AGCATTGACGGTGGTATTCCTGAAACCCACGACCGCCAGCGCGGTAAAAAGGGTGC | |
| | GTGGCATAGTGCATTCCGGACTATGAGCCATCTGAAAGAAGTCGGGATCTACTTCG | |
| | GTTGCAACACTCAAATCAATCGTTTATCGGCGTCAGAATTCCCGATTATCTATGAA | |
| | CGTATTCGCGATGCTGGGGCACGTGCGTGGCAAATTCAGCTGACGGTTCCGATGGG | |
| | CAACGCCGCGGATAACGCAGATATGCTGCTGCAACCGTATGAATTGCTCGACATCT | |
| | ATCCGATGTTAGCCCGCGTTGCCAAACGTGCGAAACAGGAAGGCGTGCGTATTCAG | |
| | GCAGGTAACAACATCGGGTACTATGGACCGTATGAGCGTCTGCTGCGTGGCAGCGA | |
| | CGAATGGACGTTTTGGCAAGGATGTGGTGCGGGCCTTAACACCCTCGGCATCGAAG | |
| | CCGACGGCAAAATCAAAGGCTGTCCATCCCTGCCGACCGCCGCGTACACCGGCGGT | |
| | AACATTCGCGATCGCCCGCTGCGGGAAATCGTCGAACAGACCGAAGAACTGAAATT | |
| | TAACTTAAAAGCTGGTACAGAACAAGGTACGGACCATATGTGGGGCTTTTGTAAAA | |
| | CCTGCGAATTCGCGGAACTCTGTCGCGGCGGATGCAGCTGGACTGCGCATGTGTTC | |
| | TTTGACCGGCGCGGCAATAATCCGTACTGCCACCATCGGGCTCTGAAACAAGCCCA | |
| | AAAAGACATTCGCGAACGCTTTTATTTAAAAGTGAAAGCAAAGGGCAACCCGTTCG | |
| | ACAATGGTGAATTTGTTATCATTGAAGAACCTTTTAACGCTCCGTTACCCGAGAAT | |
| | GACCTGCTGCACTTTAACAGTGATCACATTCAATGGCCAGAAAACTGGCAAAATAG | |
| | TGAAAGCGCGTACGCATTGGCCAAGTAATAA | |
| plpY^{a} (Expressed | ATGAACAGTAATCAGATCCCTAACAAAGTTGCAACCGCGGCACAGAAATCTGACGA | 230 |
| as plpXY) | CAGCAGCAGCGTATTACCGCGCCAGGGGTGGCAAGACAAACAAGCCTTTATTAAGG | |
| | CACTCATTAAAGCCAAACAGTCTCTCGAAATTGCCGAAATTAGCAACTTTTTAACC | |
| tgnB | ATGAAAACCATTCTGATTATTACCAATACCCTGGATCTGACCGTGGATTATATTAT | 231 |
| | TAATCGCTATAATCATACCGCTAAATTTTTTCGTCTGAATACCGATCGTTTTTTTG | |
| | ATTATGATATTAATATTACCAATAGCGGTACCAGCATTCGTAATCGTAAATCTAAT | |
| | CTGATTATTAATATTCAGGAAATTCATAGCCTGTATTATCGCAAAATTACCCTGCC | |
| | GAATCTGGATGGCTATGAAAGTAAATATTGGACCCTGATGCAGCGCGAAATGATGA | |
| | GTATTGTTGAAGGCATTGCAGAAACCGCTGGCAATTTTGCACTGACCCGTCCGTCT | |
| | GTGCTGCGCAAAGCTGATAATAAAATTGTGCAGATGAAACTGGCAGAAGAAATTGG | |
| | TTTTATTCTGCCGCAGAGTCTGATTACCAATTCAAATCAGGCGGCAGCCTCATTTT | |
| | GCAATAAAAATAATACCAGCATTGTGAAACCGCTGAGTACCGGCCGCATTCTGGGT | |
| | AAAAATAAAATTGGCATTATTCAGACCAATCTGGTTGAAACCCATGAAAATATTCA | |
| | GGGCCTGGAACTGTCTCCGGCTTATTTTCAGGATTATATTCCGAAAGATACCGAAA | |
| | TTCGTCTGACCATTGTTGGTAATAAACTGTTTGGCGCCAATATTAAATCAACCAAT | |
| | CAGGTTGATTGGCGCAAAAATGATGCACTGCTGGAATATAAACCGGCCAATATTCC | |
| | GGATAAAATTGCCAAAATGTGTCTGGAAATGATGGAAAAACTGGAAATTAATTTTG | |
| | CGGCGTTTGATTTTATTATTCGTAATGGTGATTATATTTTTCTGGAACTGAATGCC | |
| | AATGGTCAGTGGCTGTGGCTGGAAGATATTCTGAAATTTGATATTTCAAATACCAT | |
| | TATTAATTATCTGCTGGGTGAACCGATTTAATAATAA | |
| thcoK | ATGACGAGAACCAACACCGGCTATCGTTATCGCGCGTTCGGCCTGCGCATAGACTC | 232 |
| | AGATATTCCGCTGCCAGAATTAGGGGACGGTACGCGCCCTGATGGTGACGCGGATC | |
| | TGACGGTCGTCCGGTGTGGGGAAGCGGAGCCGGAATGGGCTGAAGGTGGTGGCGGG | |
| | GGTCGTCTGTATGCCGCTGAAGGCATTGTATCTTTTCGCGTGCCGCAGACGGCAGC | |
| | GTTCCGTATTACTAATGGAAATCGCATCGAGGTGCATGCCTACTCGGGGGCTGATG | |
| | AGGATCGAATACGCCTGTACGTGTTAGGGACCTGTATGGGAGCGCTGTTACTGCAA | |
| | CGTAGAATCTTACCGCTTCATGGTTCGGTCGTCGCCCGTGATGGTCGTGCGTATGC | |
| | CATAGTTGGCGAAAGCGGAGCGGGCAAATCCACGATGAGTGCAGCACTTCTCGAAC | |
| | GTGGATTCCGCCTCGTTACGGATGACGTGGCCGCCATCGTGTTCGATGAGCGTGGG | |
| | ACCCCACTGGTTATGCCGGCTTATCCACAGCAAAAACTGTGGCAGGATTCCCTGGA | |
| | CCGTCTGCAAATTGCGGGCTCGGGCCTTCGTCCGCTGTTCGAACGCGAAACGAAAT | |
| | ACGCTGTACCCGCGGATGGGGCATTCTGGCCCGAACCGGTTCCATTGGTGCACATT | |
| | TACGAACTGGTTCATAGCGATGGTCAAACGCCTGAACTGCAGCCGATTGCCAAATT | |
| | AGAGCGTTGCTATACCTTGTATCGCCACACATTTCGTAGAAGCCTGATCGTCCCCA | |
| | GCGGCTTAAGCGCCTGGCATTTTGAAACGGCAGTGAAACTTGCGGAGAAAACGGGG | |
| | ATGTACCGTCTTATGCGCCCGGCCAAAGTTTTCGCGGCTCGCGAATCTGCTCGGCT | |
| | GATTGAAACTCACGCCGATGGTGAAGTGTCACGTTAATAA | |

Wild-type Precursor Peptides

| | | |
|---|---|---|
| epiA | GAAGCAGTTAAAGAGAAGAACGATCTGTTCAACCTGGATGTTAAAGTCAACGCAAA | 233 |
| | AGAAAGTAACGATAGTGGCGCAGAACCACGCATAGCGTCGAAATTTATTTGCACAC | |
| | CAGGCTGCGCGAAAACGGGTTCGTTTAACAGCTATTGTTGTTAATAA | |
| lasA | ATGGACAAACGTGTGCGTTACGAAAAACCGAGCCTGGTGAAAGAGGGTACGTTTCG | 234 |
| | CAAAACTACCGCTGGCCTGCGGCGTCTGTTCGCTGACCAGCTGGTTGGCCGCCGTA | |
| | ACATTTAATAA | |
| paaP | ATGATTAAATTTTCTACATTGTCTCAGCGCATCAGCGCCATCACGGAAGAAAACGC | 235 |
| | CATGTACACTAAGGGTCAAGTGATCGTATTGAGCTGATAA | |
| padeA | AAAAAGCAATATAGCAAACCTAGCCTGGAGGTTCTGGACGTCCACCAGACCATGGC | 236 |
| | TGGCCCGGGCACTAGTACGCCAGACGCGTTTCAGCCAGATCCAGATGAAGATGTTC | |
| | ACTATGATTCGTAATAA | |
| palA | AAAGATCTTCTGAAGGAACTGATGTATGAAGTAGACCTCGAAGAGATGGAGAATCT | 237 |
| | TCAGGGTAGCGGGTACTCAGCCGCCCAGTGTGCCTGGATGGCGCTGAGCTGCGTCA | |
| | ATTACATCCCGGGAGTGGGATTCGGTTGTGGCGGCTACAGCGCATGTGAACTCTAC | |
| | AAGCGTTATTGTTAATAA | |
| plpA2 | ATGTCTATTGAGAGTGCAAAGGCTTTCTACCAGCGTATGACGGATGACGCATCTTT | 238 |
| | TCGTACCCCTTTTGAAGCGGAACTGTCGAAAGAGGAGCGCCAACAATTAATCAAAG | |
| | ATAGCGGATATGACTTTACTGCAGAAGAATGGCAACAGGCTATGACCGAGATCCAG | |
| | GCGGCACGCTCAAACGAGGAACTGAATGAGGAAGAACTCGAGGCAATTGCCGGGGG | |
| | CGCTGTGGCCGCAATGTATGGTGTGGTTTTCCCATGGGACAACGAGTTCCCGTGGC | |
| | CCCGCTGGGGCGGTTAATAA | |
| tgnA* | TATCGACCTTATATTGCCAAGTATGTCGAAGAACAAACTCTGCAGAATTCAACCAA | 239 |
| | CCTGGTATATGACGACATCACGCAGATCTCTTTTATCAATAAAGAAAAGAACGTGA | |
| | AAAAAATTAATCTGGGTCCCGATACTACGATCGTGACTGAAACCATCGAGAATGCG | |
| | GACCCCGATGAGTATTTCTTATAATAA | |
| thcoA | CGCAAGAAAGAATGGCAGACACCAGAACTGGAAGTACTCGATGTACGCCTCACCGC | 240 |
| | AGCGGGCCCGGGTAAAGCTAAACCGGATGCTGTGCAGCCAGACGAAGATGAAATAG | |
| | TGCACTACTCATAATAA | |

Plasmid Origins

| | | |
|---|---|---|
| pSC101 var2 | AGTAAGACGGGTAAGCCTGTTGATGATACCGCTGCCTTACTGGGTGCATTAGCCAG | 241 |
| | TCTGAATGACCTGTCACGGGATAATCCGAAGTGGTCAGACTGGAAAATCAGAGGGC | |
| | AGGAACTGCTGAACAGCAAAAAGTCAGATAGCACCACATAGCAGACCCGCCATAAA | |
| | ACGCCCTGAGAAGCCCGTGACGGGCTTTTCTTGTATTATGGGTAGTTTCCTTGCAT | |
| | GAATCCATAAAAGGCGCCTGTAGTGCCATTTACCCCCATTCACTGCCAGAGCCGTG | |
| | AGCGCAGCGAACTGAATGTCACGAAAAAGACAGCGACTCAGGTGCCTGATGGTCGG | |
| | AGACAAAAGGAATATTCAGCGATTTGCCCGAGCTTGCGAGGGTGCTACTTAAGCCT | |
| | TTAGGGTTTTAAGGTCTGTTTTGTAGAGGAGCAAACAGCGTTTGCGACATCCTTTT | |
| | GTAATACTGCGGAACTGACTAAAGTAGTGAGTTATACACAGGGCTGGGATCTATTC | |
| | TTTTTATCTTTTTTTATTCTTTCTTTATTCTATAAATTATAACCACTTGAATATAA | |
| | ACAAAAAAAACACACAAAGGTCTAGCGGAATTTACAGAGGGTCTAGCAGAATTTAC | |
| | AAGTTTTCCAGCAAAGGTCTAGCAGAATTTACAGATACCCACAACTCAAAGGAAAA | |
| | GGACTAGTAATTATCATTGACTAGCCCATCTCAATTGGTATAGTGATTAAAATCAC | |
| | CTAGACCAATTGAGATGTATGTCTGAATTAGTTGTTTTCAAAGCAAATGAACTAGC | |
| | GATTAGTCGCTATGACTTAACGGAGCATGAAACCAAGCTAATTTTATGCTGTGTGG | |
| | CACTACTCAACCCCACGATTGAAAACCCTACAAGGAAAGAACGGACGGTATCGTTC | |
| | ACTTATAACCAATACGCTCAGATGATGAACATCAGTAGGGAAAATGCTTATGGTGT | |
| | ATTAGCTAAAGCAACCAGAGAGCTGATGACGAGAACTGTGGAAATCAGGAATCCTT | |
| | TGGTTAAAGGCTTTTGGATTTTCCAGTGGACAAACTATGCCAAGTTCTCAAGCGAA | |
| | AAATTAGAATTAGTTTTTAGTGAAGAGATATTGCCTTATCTTTTCCAGTTAAAAAA | |
| | ATTCATAAAATATAATCTGGAACATGTTAAGTCTTTTGAAAACAAATACTCTATGA | |
| | GGATTTATGAGTGGTTATTAAAAGAACTAACACAAAAGAAAACTCACAAGGCAAAT | |
| | ATAGAGATTAGCCTTGATGAATTTAAGTTCATGTTAATGCTTGAAAATAACTACCA | |
| | TGAGTTTAAAAGGCTTAACCAATGGGTTTTGAAACCAATAAGTAAAGATTTAAACA | |
| | CTTACAGCAATATGAAATTGGTGGTTGATAAGCGAGGCCGCCCGACTGATACGTTG | |
| | ATTTTCCAAGTTGAACTAGATAGACAAATGGATCTCGTAACCGAACTTGAGAACAA | |
| | CCAGATAAAAATGAATGGTGACAAAATACCAACAACCATTACATCAGATTCCTACC | |
| | TACATAACGGACTAAGAAAAACACTACACGATGCTTTAACTGCAAAAATTCAGCTC | |
| | ACCAGTTTTGAGGCAAAATTTTTGAGTGACATGCAAAGTAAGTATGATCTCAATGG | |
| | TTCGTTCTCATGGCTCACGCAAAAACAACGAACCACACTAGAGAACATACTGGCTA | |
| | AATACGGAAGGATCTGAGGTTCTTATGGCTCTTGTATCTATCAGTGAAGCATCAAG | |
| | ACTAACAAACAAAAGTAGAACAACTGTTCACCGTTACATATCAAAGGGAAAACTGT | |
| | CCATATGCACAGATGAAAACGGTGTAAAAAAGATAGATACATCAGAGCTTTTACGA | |
| | GTTTTTGGTGCATTCAAAGCTGTTCACCATGAACAGATCGACAATGTAACAGATGA | |
| | ACAGCATGTAACACCTAATAGAACAGGTGAAACCAGTAAAACAAAGCAACTAGAAC | |
| | ATGAAATTGAACACCTGAGACAACTTGTTACAGCTCAACAGTCACACATAGACAGC | |
| | CTGAAACAGGCGATGCTGCTTATCGAATCAAAGCTGCCGACAACACGGGAGCCAGT | |
| | GACGCCTCCCGTGGGGAAAAAATCATGGCAATTCTGGAAGAAATAGCGCTTTCAGC | |
| | CGGCAAACCGGCTGAAGCCGGATCTGCGATTCTGATAACAAACTAGCAACACCAGA | |
| | ACAGCCCGTTTGCGGGCAGCAAAACCCGTAC | |
| p15A | TTAATAAGATGATCTTCTTGAGATCGTTTTGGTCTGCGCGTAATCTCTTGCTCTGA | 242 |
| | AAACGAAAAAACCGCCTTGCAGGGCGGTTTTTCGAAGGTTCTCTGAGCTACCAACT | |
| | CTTTGAACCGAGGTAACTGGCTTGGAGGAGCGCAGTCACCAAAACTTGTCCTTTCA | |
| | GTTTAGCCTTAACCGGCGCATGACTTCAAGACTAACTCCTCTAAATCAATTACCAG | |
| | TGGCTGCTGCCAGTGGTGCTTTTGCATGTCTTTCCGGGTTGGACTCAAGACGATAG | |
| | TTACCGGATAAGGCGCAGCGGTCGGACTGAACGGGGGGTTCGTGCATACAGTCCAG | |
| | CTTGGAGCGAACTGCCTACCCGGAACTGAGTGTCAGGCGTGGAATGAGACAAACGC | |
| | GGCCATAACAGCGGAATGACACCGGTAAACCGAAAGGCAGGAACAGGAGAGCGCAC | |
| | GAGGGAGCCGCCAGGGGAAACGCCTGGTATCTTTATAGTCCTGTCGGGTTTCGCCA | |
| | CCACTGATTTGAGCGTCAGATTTCGTGATGCTTGTCAGGGGGGCGGAGCCTATGGA | |
| | AAAACGGCTTTGCCGCGGCCCTCTCACTTCCCTGTTAAGTATCTTCCTGGCATCTT | |
| | CCAGGAAATCTCCGCCCCGTTCGTAAGCCATTTCCGCTCGCCGCAGTCGAACGACC | |
| | GAGCGTAGCGAGTCAGTGAGCGAGGAAGCGGAATATATCCTGTATCACATATTCTG | |
| | CTGACGCACCGGTGCAGCCTTTTTTCTCCTGCCACATGAAGCACTTCACTGACACC | |
| | CTCATCAGTGCCAACATAGTAAGCCAGTATACACTCCGCTA | |

^{a}PlpXY genes were synthesized/expressed as RBS_{PLpX} + PlpX + RBS_{PlpY} + PlpY.

TABLE 10
Plasmid backbone/chassis sequences.

| | | SEQ |
|---|---|---|
| | | ID |
| Name | Sequence^{a} | NO |
| N-term SUMO | CACATTTCCCCGAAAAGTGCCACCTGACGTCTAAGAAACCATTATTATCATGACATTAA | 243 |
| Backbone 2 | CCTATAAAAATAGGCGTATCACGAGGCAGAATTTCAGATAAAAAAAATCCTTAGCTTTC | |
| | GCTAAGGATGATTTCTGGAATTCGCGGCCGCTTCTAGAGGGAGAACGATCGTTGGCTGa | |
| | atcataaaaaatttatttgctttgtgagcggataacaattataatagattcaattgtga | |
| | 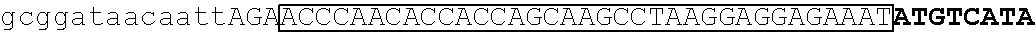 | |
| | TTACCACCATCACCATCATCACGGGTCCCTGCAGGACTCAGAAGTCAATCAAGAAGCTA | |
| | AGCCAGAGGTCAAGCCAGAAGTCAAGCCTGAGACTCACATCAATTTAAAGGTGTCCGAT | |
| | GGATCTTCAGAGATCTTCTTCAAGATCAAAAAGACCACTCCTTTAAGAAGGCTGATGGA | |
| | AGCGTTCGCTAAAAGACAGGGTAAGGAAATGGACTCCTTAAGATTCTTGTACGACGGTA | |
| | TTAGAATTCAAGCTGATCAGGCCCCTGAAGATTTGGACATGGAGGATAACGATATTATT | |
| | GAGGCTCACCGCGAACAGATTGGAGGTTGCATGTCATATTACGACTCCATTCCCACAAG | |
| | CGAGAACTTGTACTTTCAAGGGTGCATGAGCAAAGGAGAAGAACTTTTCACTGGAGTTG | |
| | TCCCAATTCTTGTTGAATTAGATGGTGATGTTAATGGGCACAAATTTTCTGTCCGTGGA | |
| | GAGGGTGAAGGTGATGCTACAAACGGAAAACTCACCCTTAAATTTATTTGCACTACTGG | |
| | AAAACTACCTGTTCCGTGGCCAACACTTGTCACTACTCTGACCTATGGTGTTCAATGCT | |
| | TTTCCCGTTATCCGGATCACATGAAACGGCATGACTTTTTCAAGAGTGCCATGCCCGAA | |
| | GGTTATGTACAGGAACGCACTATATCTTTCAAAGATGACGGGACCTACAAGACGCGTGC | |
| | TGAAGTCAAGTTTGAAGGTGATACCCTTGTTAATCGTATCGAGTTAAAGGGTATTGATT | |
| | TTAAAGAAGATGGAAACATTCTTGGACACAAACTCGAGTACAACTTTAACTCACACAAT | |
| | GTATACATCACGGCAGACAAACAAAAGAATGGAATCAAAGCTAACTTCAAAATTCGCCA | |
| | CAACGTTGAAGATGGTTCCGTTCAACTAGCAGACCATTATCAACAAAATACTCCAATTG | |
| | GCGATGGCCCTGTCCTTTTACCAGACAACCATTACCTGTCGACACAATCTGTCCTTTCG | |
| | AAAGATCCCAACGAAAAGCGTGACCACATGGTCCTTCTTGAGTTTGTAACTGCTGCTGG | |
| | GATTACACATGGCATGGATGAGCTCTACAAATAATTCAGCCAAAAAACTTAAGACCGCC | |
| | GGTCTTGTCCACTACCTTGCAGTAATGCGGTGGACAGGATCGGCGGTTTTCTTTTCTCT | |
| | TCTCAACCAATGgcggcgcgccatcgaatggcgcaaaacctttcgcggtatggcatgat | |
| | 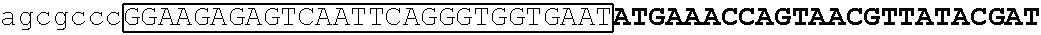 | |
| | GTCGCAGAGTATGCCGGTGTCTCTTATCAGACCGTTTCCCGCGTGGTGAACCAGGCCAG | |
| | CCACGTTTCTGCGAAAACGCGGGAAAAAGTGGAAGCGGCGATGGCGGAGCTGAATTACA | |
| | TTCCCAACCGCGTGGCACAACAACTGGCGGGCAAACAGTCGTTGCTTATTGGCGTTGCC | |
| | ACCTCCAGTCTGGCCCTGCACGCGCCGTCGCAAATTGTCGCGGCGATTAAATCTCGCGC | |
| | CGATCAACTGGGTGCCAGCGTGGTGGTGTCGATGGTAGAACGAAGCGGCGTCGAAGCCT | |
| | GTAAAGCGGCGGTGCACAATCTTCTCGCGCAACGCGTCAGTGGGCTGATCATTAACTAT | |
| | CCGCTGGATGACCAGGATGCCATTGCTGTGGAAGCTGCCTGCACTAATGTTCCGGCGTT | |
| | ATTTCTTGATGTCTCTGACCAGACACCCATCAACAGTATTATTTTCTCCCATGAGGACG | |
| | GTACGCGACTGGGCGTGGAGCATCTGGTCGCATTGGGTCACCAGCAAATCGCGCTGTTA | |
| | GCGGGCCCATTAAGTTCTGTCTCGGCGCGTCTGCGTCTGGCTGGCTGGCATAAATATCT | |
| | CACTCGCAATCAAATTCAGCCGATAGCGGAACGGGAAGGCGACTGGAGTGCCATGTCCG | |
| | GTTTTCAACAAACCATGCAAATGCTGAATGAGGGCATCGTTCCCACTGCGATGCTGGTT | |
| | GCCAACGATCAGATGGCGCTGGGCGCAATGCGCGCCATTACCGAGTCCGGGCTGCGCGT | |
| | TGGTGCGGATATCTCGGTAGTGGGATACGACGATACCGAAGATAGCTCATGTTATATCC | |
| | CGCCGTTAACCACCATCAAACAGGATTTTCGCCTGCTGGGGCAAACCAGCGTGGACCGC | |
| | TTGCTGCAACTCTCTCAGGGCCAGGCGGTGAAGGGCAATCAGCTGTTGCCAGTCTCACT | |
| | GGTGAAAAGAAAAACCACCCTGGCGCCCAATACGCAAACCGCCTCTCCCCGCGCGTTGG | |
| | CCGATTCATTAATGCAGCTGGCACGACAGGTTTCCCGACTGGAAAGCGGGCAGTGATAA | |
| | TTGGTAACGAATCAGACAATTGACGGCTCGAGGGAGTAGCATAGGGTTTGCAGAATCCC | |
| | TGCTTCGTCCATTTGACAGGCACATTATGCATCGATGATAAGCTGTCAAACATGAGCAC | |
| | GCTTACTAGTAGCGGCCGCTGCAGTCCGGCAAAAAAGGGCAAGGTGTCACCACCCTGCC | |
| | CTTTTTCTTTAAAACCGAAAAGATTACTTCGCGTTATGCAGGCTTCCTCGCTCACTGAC | |
| | TCGCTGCGCTCGGTCGTTCGGCTGCGGCGAGCGGTATCAGCTCACTCAAAGGCGGTAAT | |
| | GACAGTAAGACGGGTAAGCCTGTTGATGATACCGCTGCCTTACTGGGTGCATTAGCCAG | |
| | TCTGAATGACCTGTCACGGGATAATCCGAAGTGGTCAGACTGGAAAATCAGAGGGCAGG | |
| | AACTGCTGAACAGCAAAAAGTCAGATAGCACCACATAGCAGACCCGCCATAAAACGCCC | |
| | TGAGAAGCCCGTGACGGGCTTTTCTTGTATTATGGGTAGTTTCCTTGCATGAATCCATA | |
| | AAAGGCGCCTGTAGTGCCATTTACCCCCATTCACTGCCAGAGCCGTGAGCGCAGCGAAC | |
| | TGAATGTCACGAAAAAGACAGCGACTCAGGTGCCTGATGGTCGGAGACAAAAGGAATAT | |
| | TCAGCGATTTGCCCGAGCTTGCGAGGGTGCTACTTAAGCCTTTAGGGTTTTAAGGTCTG | |
| | TTTTGTAGAGGAGCAAACAGCGTTTGCGACATCCTTTTGTAATACTGCGGAACTGACTA | |
| | AAGTAGTGAGTTATACACAGGGCTGGGATCTATTCTTTTTATCTTTTTTTATTCTTTCT | |
| | TTATTCTATAAATTATAACCACTTGAATATAAACAAAAAAAACACACAAAGGTCTAGCG | |
| | GAATTTACAGAGGGTCTAGCAGAATTTACAAGTTTTCCAGCAAAGGTCTAGCAGAATTT | |
| | ACAGATACCCACAACTCAAAGGAAAAGGACTAGTAATTATCATTGACTAGCCCATCTCA | |
| | ATTGGTATAGTGATTAAAATCACCTAGACCAATTGAGATGTATGTCTGAATTAGTTGTT | |
| | TTCAAAGCAAATGAACTAGCGATTAGTCGCTATGACTTAACGGAGCATGAAACCAAGCT | |
| | AATTTTATGCTGTGTGGCACTACTCAACCCCACGATTGAAAACCCTACAAGGAAAGAAC | |
| | GGACGGTATCGTTCACTTATAACCAATACGCTCAGATGATGAACATCAGTAGGGAAAAT | |
| | GCTTATGGTGTATTAGCTAAAGCAACCAGAGAGCTGATGACGAGAACTGTGGAAATCAG | |
| | GAATCCTTTGGTTAAAGGCTTTTGGATTTTCCAGTGGACAAACTATGCCAAGTTCTCAA | |
| | GCGAAAAATTAGAATTAGTTTTTAGTGAAGAGATATTGCCTTATCTTTTCCAGTTAAAA | |
| | AAATTCATAAAATATAATCTGGAACATGTTAAGTCTTTTGAAAACAAATACTCTATGAG | |
| | GATTTATGAGTGGTTATTAAAAGAACTAACACAAAAGAAAACTCACAAGGCAAATATAG | |
| | AGATTAGCCTTGATGAATTTAAGTTCATGTTAATGCTTGAAAATAACTACCATGAGTTT | |
| | AAAAGGCTTAACCAATGGGTTTTGAAACCAATAAGTAAAGATTTAAACACTTACAGCAA | |
| | TATGAAATTGGTGGTTGATAAGCGAGGCCGCCCGACTGATACGTTGATTTTCCAAGTTG | |
| | AACTAGATAGACAAATGGATCTCGTAACCGAACTTGAGAACAACCAGATAAAAATGAAT | |
| | GGTGACAAAATACCAACAACCATTACATCAGATTCCTACCTACATAACGGACTAAGAAA | |
| | AACACTACACGATGCTTTAACTGCAAAAATTCAGCTCACCAGTTTTGAGGCAAAATTTT | |
| | TGAGTGACATGCAAAGTAAGTATGATCTCAATGGTTCGTTCTCATGGCTCACGCAAAAA | |
| | CAACGAACCACACTAGAGAACATACTGGCTAAATACGGAAGGATCTGAGGTTCTTATGG | |
| | CTCTTGTATCTATCAGTGAAGCATCAAGACTAACAAACAAAAGTAGAACAACTGTTCAC | |
| | CGTTACATATCAAAGGGAAAACTGTCCATATGCACAGATGAAAACGGTGTAAAAAAGAT | |
| | AGATACATCAGAGCTTTTACGAGTTTTTGGTGCATTCAAAGCTGTTCACCATGAACAGA | |
| | TCGACAATGTAACAGATGAACAGCATGTAACACCTAATAGAACAGGTGAAACCAGTAAA | |
| | ACAAAGCAACTAGAACATGAAATTGAACACCTGAGACAACTTGTTACAGCTCAACAGTC | |
| | ACACATAGACAGCCTGAAACAGGCGATGCTGCTTATCGAATCAAAGCTGCCGACAACAC | |
| | GGGAGCCAGTGACGCCTCCCGTGGGGAAAAAATCATGGCAATTCTGGAAGAAATAGCGC | |
| | TTTCAGCCGGCAAACCGGCTGAAGCCGGATCTGCGATTCTGATAACAAACTAGCAACAC | |
| | CAGAACAGCCCGTTTGCGGGCAGCAAAACCCGTACCGATTATCAAAAAGGATCTTCACC | |
| | TAGATCCTTTTAAATTAAAAATGAAGTTTTAAATCAATCTAAAGTATATATGAGTAAAC | |
| | TTGGTCTGACAGTTACCAATGCTTAATCAGTGAGGCACCTATCTCAGCGATCTGTCTAT | |
| | TTCGTTCATCCATAGTTGCCTGACTCCCCGTCGTGTAGATAACTACGATACGGGAGGGC | |
| | TTACCATCTGGCCCCAGTGCTGCAATGATACCGCGAGAACCACGCTCACCGGCTCCAGA | |
| | TTTATCAGCAATAAACCAGCCAGCCGGAAGGGCCGAGCGCAGAAGTGGTCCTGCAACTT | |
| | TATCCGCCTCCATCCAGTCTATTAATTGTTGCCGGGAAGCTAGAGTAAGTAGTTCGCCA | |
| | GTTAATAGTTTGCGCAACGTTGTTGCCATTGCTACAGGCATCGTGGTGTCACGCTCGTC | |
| | GTTTGGTATGGCTTCATTCAGCTCCGGTTCCCAACGATCAAGGCGAGTTACATGATCCC | |
| | CCATGTTGTGCAAAAAAGCGGTTAGCTCCTTCGGTCCTCCGATCGTTGTCAGAAGTAAG | |
| | TTGGCCGCAGTGTTATCACTCATGGTTATGGCAGCACTGCATAATTCTCTTACTGTCAT | |
| | GCCATCCGTAAGATGCTTTTCTGTGACTGGTGAGTACTCAACCAAGTCATTCTGAGAAT | |
| | AGTGTATGCGGCGACCGAGTTGCTCTTGCCCGGCGTCAATACGGGATAATACCGCGCCA | |
| | CATAGCAGAACTTTAAAAGTGCTCATCATTGGAAAACGTTCTTCGGGGCGAAAACTCTC | |
| | AAGGATCTTACCGCTGTTGAGATCCAGTTCGATATAACCCACTCGTGCACCCAACTGAT | |
| | CTTCAGCATCTTTTACTTTCACCAGCGTTTCTGGGTGAGCAAAAACAGGAAGGCAAAAT | |
| | GCCGCAAAAAAGGGAATAAGGGCGACACGGAAATGTTGAATACTCATACTCTTCCTTTT | |
| | TCAATATTATTGAAGCATTTATCAGGGTTATTGTCTCATGAGCGGATACATATTTGAAT | |
| | GTATTTAGAAAAATAAACAAATAGGGGTTCCGCG | |
| N-term SUMO | CACATTTCCCCGAAAAGTGCCACCTGACGTCTAAGAAACCATTATTATCATGACATTAA | 244 |
| Backbone 3 | CCTATAAAAATAGGCGTATCACGAGGCAGAATTTCAGATAAAAAAAATCCTTAGCTTTC | |
| | GCTAAGGATGATTTCTGGAATTCGCGGCCGCTTCTAGAGGGAGGGTCTCAGTGCAACGA | |
| | TCGTTGGCTGaatcataaaaaatttatttgctttgtgagcggataacaattataataga | |
| | 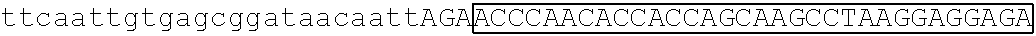 | |
| | 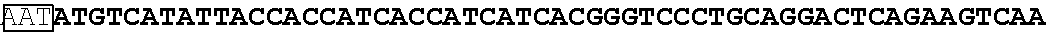 | |
| | TCAAGAAGCTAAGCCAGAGGTCAAGCCAGAAGTCAAGCCTGAGACTCACATCAATTTAA | |
| | AGGTGTCCGATGGATCTTCAGAGATCTTCTTCAAGATCAAAAAGACCACTCCTTTAAGA | |
| | AGGCTGATGGAAGCGTTCGCTAAAAGACAGGGTAAGGAAATGGACTCCTTAAGATTCTT | |
| | GTACGACGGTATTAGAATTCAAGCTGATCAGGCCCCTGAAGATTTGGACATGGAGGATA | |
| | ACGATATTATTGAGGCTCACCGCGAACAGATTGGAGGTTGCATGTCATATTACGACTCC | |
| | ATTCCCACAAGCGAGAACTTGTACTTTCAAGGGTGCATGAGCAAAGGAGAAGAACTTTT | |
| | CACTGGAGTTGTCCCAATTCTTGTTGAATTAGATGGTGATGTTAATGGGCACAAATTTT | |
| | CTGTCCGTGGAGAGGGTGAAGGTGATGCTACAAACGGAAAACTCACCCTTAAATTTATT | |
| | TGCACTACTGGAAAACTACCTGTTCCGTGGCCAACACTTGTCACTACTCTGACCTATGG | |
| | TGTTCAATGCTTTTCCCGTTATCCGGATCACATGAAACGGCATGACTTTTTCAAGAGTG | |
| | CCATGCCCGAAGGTTATGTACAGGAACGCACTATATCTTTCAAAGATGACGGGACCTAC | |
| | AAGACGCGTGCTGAAGTCAAGTTTGAAGGTGATACCCTTGTTAATCGTATCGAGTTAAA | |
| | GGGTATTGATTTTAAAGAAGATGGAAACATTCTTGGACACAAACTCGAGTACAACTTTA | |
| | ACTCACACAATGTATACATCACGGCAGACAAACAAAAGAATGGAATCAAAGCTAACTTC | |
| | AAAATTCGCCACAACGTTGAAGATGGTTCCGTTCAACTAGCAGACCATTATCAACAAAA | |
| | TACTCCAATTGGCGATGGCCCTGTCCTTTTACCAGACAACCATTACCTGTCGACACAAT | |
| | CTGTCCTTTCGAAAGATCCCAACGAAAAGCGTGACCACATGGTCCTTCTTGAGTTTGTA | |
| | ACTGCTGCTGGGATTACACATGGCATGGATGAGCTCTACAAATAATTCAGCCAAAAAAC | |
| | TTAAGACCGCCGGTCTTGTCCACTACCTTGCAGTAATGCGGTGGACAGGATCGGCGGTT | |
| | TTCTTTTCTCTTCTCAACAAGTGAGACCATGGgcggcgcgccatcgaatggcgcaaaac | |
| | 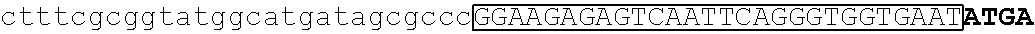 | |
| | AACCAGTAACGTTATACGATGTCGCAGAGTATGCCGGTGTCTCTTATCAGACCGTTTCC | |
| | CGCGTGGTGAACCAGGCCAGCCACGTTTCTGCGAAAACGCGGGAAAAAGTGGAAGCGGC | |
| | GATGGCGGAGCTGAATTACATTCCCAACCGCGTGGCACAACAACTGGCGGGCAAACAGT | |
| | CGTTGCTTATTGGCGTTGCCACCTCCAGTCTGGCCCTGCACGCGCCGTCGCAAATTGTC | |
| | GCGGCGATTAAATCTCGCGCCGATCAACTGGGTGCCAGCGTGGTGGTGTCGATGGTAGA | |
| | ACGAAGCGGCGTCGAAGCCTGTAAAGCGGCGGTGCACAATCTTCTCGCGCAACGCGTCA | |
| | GTGGGCTGATCATTAACTATCCGCTGGATGACCAGGATGCCATTGCTGTGGAAGCTGCC | |
| | TGCACTAATGTTCCGGCGTTATTTCTTGATGTCTCTGACCAGACACCCATCAACAGTAT | |
| | TATTTTCTCCCATGAGGACGGTACGCGACTGGGCGTGGAGCATCTGGTCGCATTGGGTC | |
| | ACCAGCAAATCGCGCTGTTAGCGGGCCCATTAAGTTCTGTCTCGGCGCGTCTGCGTCTG | |
| | GCTGGCTGGCATAAATATCTCACTCGCAATCAAATTCAGCCGATAGCGGAACGGGAAGG | |
| | CGACTGGAGTGCCATGTCCGGTTTTCAACAAACCATGCAAATGCTGAATGAGGGCATCG | |
| | TTCCCACTGCGATGCTGGTTGCCAACGATCAGATGGCGCTGGGCGCAATGCGCGCCATT | |
| | ACCGAGTCCGGGCTGCGCGTTGGTGCGGATATCTCGGTAGTGGGATACGACGATACCGA | |
| | AGATAGCTCATGTTATATCCCGCCGTTAACCACCATCAAACAGGATTTTCGCCTGCTGG | |
| | GGCAAACCAGCGTGGACCGCTTGCTGCAACTCTCTCAGGGCCAGGCGGTGAAGGGCAAT | |
| | CAGCTGTTGCCAGTCTCACTGGTGAAAAGAAAAACCACCCTGGCGCCCAATACGCAAAC | |
| | CGCCTCTCCCCGCGCGTTGGCCGATTCATTAATGCAGCTGGCACGACAGGTTTCCCGAC | |
| | TGGAAAGCGGGCAGTGATAATTGGTAACGAATCAGACAATTGACGGCTCGAGGGAGTAG | |
| | CATAGGGTTTGCAGAATCCCTGCTTCGTCCATTTGACAGGCACATTATGCATCGATGAT | |
| | AAGCTGTCAAACATGAGCACGCTTACTAGTAGCGGCCGCTGCAGTCCGGCAAAAAAGGG | |
| | CAAGGTGTCACCACCCTGCCCTTTTTCTTTAAAACCGAAAAGATTACTTCGCGTTATGC | |
| | AGGCTTCCTCGCTCACTGACTCGCTGCGCTCGGTCGTTCGGCTGCGGCGAGCGGTATCA | |
| | GCTCACTCAAAGGCGGTAATGACAGTAAGACGGGTAAGCCTGTTGATGATACCGCTGCC | |
| | TTACTGGGTGCATTAGCCAGTCTGAATGACCTGTCACGGGATAATCCGAAGTGGTCAGA | |
| | CTGGAAAATCAGAGGGCAGGAACTGCTGAACAGCAAAAAGTCAGATAGCACCACATAGC | |
| | AGACCCGCCATAAAACGCCCTGAGAAGCCCGTGACGGGCTTTTCTTGTATTATGGGTAG | |
| | TTTCCTTGCATGAATCCATAAAAGGCGCCTGTAGTGCCATTTACCCCCATTCACTGCCA | |
| | GAGCCGTGAGCGCAGCGAACTGAATGTCACGAAAAAGACAGCGACTCAGGTGCCTGATG | |
| | GTCGGAGACAAAAGGAATATTCAGCGATTTGCCCGAGCTTGCGAGGGTGCTACTTAAGC | |
| | CTTTAGGGTTTTAAGGTCTGTTTTGTAGAGGAGCAAACAGCGTTTGCGACATCCTTTTG | |
| | TAATACTGCGGAACTGACTAAAGTAGTGAGTTATACACAGGGCTGGGATCTATTCTTTT | |
| | TATCTTTTTTTATTCTTTCTTTATTCTATAAATTATAACCACTTGAATATAAACAAAAA | |
| | AAACACACAAAGGTCTAGCGGAATTTACAGAGGGTCTAGCAGAATTTACAAGTTTTCCA | |
| | GCAAAGGTCTAGCAGAATTTACAGATACCCACAACTCAAAGGAAAAGGACTAGTAATTA | |
| | TCATTGACTAGCCCATCTCAATTGGTATAGTGATTAAAATCACCTAGACCAATTGAGAT | |
| | GTATGTCTGAATTAGTTGTTTTCAAAGCAAATGAACTAGCGATTAGTCGCTATGACTTA | |
| | ACGGAGCATGAAACCAAGCTAATTTTATGCTGTGTGGCACTACTCAACCCCACGATTGA | |
| | AAACCCTACAAGGAAAGAACGGACGGTATCGTTCACTTATAACCAATACGCTCAGATGA | |
| | TGAACATCAGTAGGGAAAATGCTTATGGTGTATTAGCTAAAGCAACCAGAGAGCTGATG | |
| | ACGAGAACTGTGGAAATCAGGAATCCTTTGGTTAAAGGCTTTTGGATTTTCCAGTGGAC | |
| | AAACTATGCCAAGTTCTCAAGCGAAAAATTAGAATTAGTTTTTAGTGAAGAGATATTGC | |
| | CTTATCTTTTCCAGTTAAAAAAATTCATAAAATATAATCTGGAACATGTTAAGTCTTTT | |
| | GAAAACAAATACTCTATGAGGATTTATGAGTGGTTATTAAAAGAACTAACACAAAAGAA | |
| | AACTCACAAGGCAAATATAGAGATTAGCCTTGATGAATTTAAGTTCATGTTAATGCTTG | |
| | AAAATAACTACCATGAGTTTAAAAGGCTTAACCAATGGGTTTTGAAACCAATAAGTAAA | |
| | GATTTAAACACTTACAGCAATATGAAATTGGTGGTTGATAAGCGAGGCCGCCCGACTGA | |
| | TAGGTTGATTTTCCAAGTTGAACTAGATAGACAAATGGATCTCGTAACCGAACTTGAGA | |
| | ACAACCAGATAAAAATGAATGGTGACAAAATACCAACAACCATTACATCAGATTCCTAC | |
| | CTACATAACGGACTAAGAAAAACACTACACGATGCTTTAACTGCAAAAATTCAGCTCAC | |
| | CAGTTTTGAGGCAAAATTTTTGAGTGACATGCAAAGTAAGTATGATCTCAATGGTTCGT | |
| | TCTCATGGCTCACGCAAAAACAACGAACCACACTAGAGAACATACTGGCTAAATACGGA | |
| | AGGATCTGAGGTTCTTATGGCTCTTGTATCTATCAGTGAAGCATCAAGACTAACAAACA | |
| | AAAGTAGAACAACTGTTCACCGTTACATATCAAAGGGAAAACTGTCCATATGCACAGAT | |
| | GAAAACGGTGTAAAAAAGATAGATACATCAGAGCTTTTACGAGTTTTTGGTGCATTCAA | |
| | AGCTGTTCACCATGAACAGATCGACAATGTAACAGATGAACAGCATGTAACACCTAATA | |
| | GAACAGGTGAAACCAGTAAAACAAAGCAACTAGAACATGAAATTGAACACCTGAGACAA | |
| | CTTGTTACAGCTCAACAGTCACACATAGACAGCCTGAAACAGGCGATGCTGCTTATCGA | |
| | ATCAAAGCTGCCGACAACACGGGAGCCAGTGACGCCTCCCGTGGGGAAAAAATCATGGC | |
| | AATTCTGGAAGAAATAGCGCTTTCAGCCGGCAAACCGGCTGAAGCCGGATCTGCGATTC | |
| | TGATAACAAACTAGCAACACCAGAACAGCCCGTTTGCGGGCAGCAAAACCCGTACCGAT | |
| | TATCAAAAAGGATCTTCACCTAGATCCTTTTAAATTAAAAATGAAGTTTTAAATCAATC | |
| | TAAAGTATATATGAGTAAACTTGGTCTGACAGTTACCAATGCTTAATCAGTGAGGCACC | |
| | TATCTCAGCGATCTGTCTATTTCGTTCATCCATAGTTGCCTGACTCCCCGTCGTGTAGA | |
| | TAACTACGATACGGGAGGGCTTACCATCTGGCCCCAGTGCTGCAATGATACCGCGAGAA | |
| | CCACGCTCACCGGCTCCAGATTTATCAGCAATAAACCAGCCAGCCGGAAGGGCCGAGCG | |
| | CAGAAGTGGTCCTGCAACTTTATCCGCCTCCATCCAGTCTATTAATTGTTGCCGGGAAG | |
| | CTAGAGTAAGTAGTTCGCCAGTTAATAGTTTGCGCAACGTTGTTGCCATTGCTACAGGC | |
| | ATCGTGGTGTCACGCTCGTCGTTTGGTATGGCTTCATTCAGCTCCGGTTCCCAACGATC | |
| | AAGGCGAGTTACATGATCCCCCATGTTGTGCAAAAAAGCGGTTAGCTCCTTCGGTCCTC | |
| | CGATCGTTGTCAGAAGTAAGTTGGCCGCAGTGTTATCACTCATGGTTATGGCAGCACTG | |
| | CATAATTCTCTTACTGTCATGCCATCCGTAAGATGCTTTTCTGTGACTGGTGAGTACTC | |
| | AACCAAGTCATTCTGAGAATAGTGTATGCGGCGACCGAGTTGCTCTTGCCCGGCGTCAA | |
| | TACGGGATAATACCGCGCCACATAGCAGAACTTTAAAAGTGCTCATCATTGGAAAACGT | |
| | TCTTCGGGGCGAAAACTCTCAAGGATCTTACCGCTGTTGAGATCCAGTTCGATATAACC | |
| | CACTCGTGCACCCAACTGATCTTCAGCATCTTTTACTTTCACCAGCGTTTCTGGGTGAG | |
| | CAAAAACAGGAAGGCAAAATGCCGCAAAAAAGGGAATAAGGGCGACACGGAAATGTTGA | |
| | ATACTCATACTCTTCCTTTTTCAATATTATTGAAGCATTTATCAGGGTTATTGTCTCAT | |
| | GAGCGGATACATATTTGAATGTATTTAGAAAAATAAACAAATAGGGGTTCCGCG | |
| Cumate | AACACCCCTTGTATTACTGTTTATGTAAGCAGACAGTTTTATTGTTCATGATGATATAT | 245 |
| Modifying | TTTTATCTTGTGCAATGTACATCAGAGATTTTGAGACACAACCAATTATTGAAGGCCTC | |
| Enzyme | CCTAACGGGGGGCCTTTTTTTGTTTCTGGTCTCCCAAGCGCTTAACGATCGTTGGCTGa | |
| Backbone | acaaacagacaatctggtctgtttgtattatggaaaatttttctgtataatagattcaa | |
| | caaacagacaatctggtctgtttgtattatCAGCGGTCAACGCATGTGCTTTGCGTTCT | |
| | 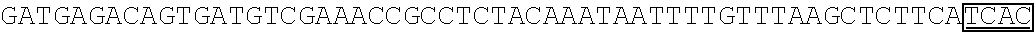 | |
| | 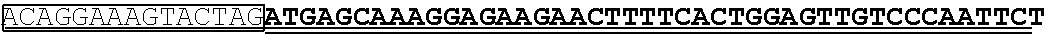 | |
| | TGTTGAATTAGATGGTGATGTTAATGGGCACAAATTTTCTGTCCGTGGAGAGGGTGAAG | |
| | GTGATGCTACAAACGGAAAACTCACCCTTAAATTTATTTGCACTACTGGAAAACTACCT | |
| | GTTCCGTGGCCAACACTTGTCACTACTCTGACCTATGGTGTTCAATGCTTTTCCCGTTA | |
| | TCCGGATCACATGAAACGGCATGACTTTTTCAAGAGTGCCATGCCCGAAGGTTATGTAC | |
| | AGGAACGCACTATATCTTTCAAAGATGACGGGACCTACAAGACGCGTGCTGAAGTCAAG | |
| | TTTGAAGGTGATACCCTTGTTAATCGTATCGAGTTAAAGGGTATTGATTTTAAAGAAGA | |
| | TGGAAACATTCTTGGACACAAACTCGAGTACAACTTTAACTCACACAATGTATACATCA | |
| | CGGCAGACAAACAAAAGAATGGAATCAAAGCTAACTTCAAAATTCGCCACAACGTTGAA | |
| | GATGGTTCCGTTCAACTAGCAGACCATTATCAACAAAATACTCCAATTGGCGATGGCCC | |
| | TGTCCTTTTACCAGACAACCATTACCTGTCGACACAATCTGTCCTTTCGAAAGATCCCA | |
| | ACGAAAAGCGTGACCACATGGTCCTTCTTGAGTTTGTAACTGCTGCTGGGATTACACAT | |
| | GGCATGGATGAGCTCTACAAATAATGAAGAGCGCAGAGGTGGTTGTGTTGCGAAAAAAA | |
| | AAAAAAACACCCTAACGGGTGTTTTTTTTTTTTTGGTGTCCCCACGTGTGGCGCTGGAG | |
| | ACCGTCCAATGgcggcgcgccatcgaatggtgcaaaacctttcgcggtatggcatgata | |
| | 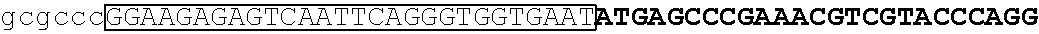 | |
| | CAGAACGTGCAATGGAAACCCAGGGTAAACTGATTGCAGCAGCACTGGGTGTTCTGCGT | |
| | GAAAAAGGTTATGCAGGTTTTCGTATTGCAGATGTTCCGGGTGCAGCCGGTGTTAGCCG | |
| | TGGTGCACAGAGCCATCATTTTCCGACCAAACTGGAACTGCTGCTGGCAACCTTTGAAT | |
| | GGCTGTATGAGCAGATTACCGAACGTAGCCGTGCACGTCTGGCAAAACTGAAACCGGAA | |
| | GATGATGTTATTCAGCAGATGCTGGATGATGCAGCAGATTTTTTTCTGGATGATGATTT | |
| | TAGCATCGGCCTGGATCTGATTGTTGCAGCAGATCGTGATCCGGCACTGCGTGAAGGTA | |
| | TTCTGCGTACCGTTGAACGTAATCGTTTTGTTGTTGAAGATATGTGGCTGGGTGTGCTG | |
| | GTGAGCCGTGGTCTGAGCCGTGATGATGCCGAAGATATTCTGTGGCTGATTTTTAACAG | |
| | CGTTCGTGGTCTGACAGTTCGTAGCCTGTGGCAGAAAGATAAAGAACGTTTTGAACGTG | |
| | TGCGTAATAGCACCCTGGAAATTGCACGTGAACGTTATGCAAAATTCAAACGTTGATAA | |
| | GGATCCTAATTGGTAACGAATCAGACAATTGACGGCTCGAGGGAGTAGCATAGGGTTTG | |
| | CAGAATCCCTGCTTCGTCCATTTGACAGGCACATTATGCATCGATGATAAGCTGTCAAA | |
| | CATGAGCAGATCCTCTACGCCGGACGCATCGTGGCCGGCATCACCGGCGCCACAGGTGC | |
| | GGTTGCTGGCGCCTATATCGCCGACATCACCGATGGGGAAGATCGGGCTCGCCACTTCG | |
| | GGCTCATGAGCAAATATTTTATCTGAGGTGCTTCCTCGCTCACTGACTCGCTGCACGAG | |
| | GCAGACCTCAGCGCTAGCGGAGTGTATACTGGCTTACTATGTTGGCACTGATGAGGGTG | |
| | TCAGTGAAGTGCTTCATGTGGCAGGAGAAAAAAGGCTGCACCGGTGCGTCAGCAGAATA | |
| | TGTGATACAGGATATATTCCGCTTCCTCGCTCACTGACTCGCTACGCTCGGTCGTTCGA | |
| | CTGCGGCGAGCGGAAATGGCTTACGAACGGGGCGGAGATTTCCTGGAAGATGCCAGGAA | |
| | GATACTTAACAGGGAAGTGAGAGGGCCGCGGCAAAGCCGTTTTTCCATAGGCTCCGCCC | |
| | CCCTGACAAGCATCACGAAATCTGACGCTCAAATCAGTGGTGGCGAAACCCGACAGGAC | |
| | TATAAAGATACCAGGCGTTTCCCCTGGCGGCTCCCTCGTGCGCTCTCCTGTTCCTGCCT | |
| | TTCGGTTTACCGGTGTCATTCCGCTGTTATGGCCGCGTTTGTCTCATTCCACGCCTGAC | |
| | ACTCAGTTCCGGGTAGGCAGTTCGCTCCAAGCTGGACTGTATGCACGAACCCCCCGTTC | |
| | AGTCCGACCGCTGCGCCTTATCCGGTAACTATCGTCTTGAGTCCAACCCGGAAAGACAT | |
| | GCAAAAGCACCACTGGCAGCAGCCACTGGTAATTGATTTAGAGGAGTTAGTCTTGAAGT | |
| | CATGCGCCGGTTAAGGCTAAACTGAAAGGACAAGTTTTGGTGACTGCGCTCCTCCAAGC | |
| | CAGTTACCTCGGTTCAAAGAGTTGGTAGCTCAGAGAACCTTCGAAAAACCGCCCTGCAA | |
| | GGCGGTTTTTTCGTTTTCAGAGCAAGAGATTACGCGCAGACCAAAACGATCTCAAGAAG | |
| | ATCATCTTATTAAGGGGTCTGACGCTCAGTGGAACGAAAAATCAATCTAAAGTATATAT | |
| | GAGTAAACTTGGTCTGACAGTTACCTTAGAAAAACTCATCGAGCATCAAATGAAACTGC | |
| | AATTTATTCATATCAGGATTATCAATACCATATTTTTGAAAAAGCCGTTTCTGTAATGA | |
| | AGGAGAAAACTCACCGAGGCAGTTCCATAGGATGGCAAGATCCTGGTATCGGTCTGCGA | |
| | TTCCGACTCGTCCAACATCAATACAACCTATTAATTTCCCCTCGTCAAAAATAAGGTTA | |
| | TCAAGTGAGAAATCACCATGAGTGACGACTGAATCCGGTGAGAATGGCAAAAGCTTATG | |
| | CATTTCTTTCCAGACTTGTTCAACAGGCCAGCCATTACGCTCGTCATCAAAATCACTCG | |
| | CATCAACCAAACCGTTATTCATTCGTGATTGCGCCTGAGCGAGACGAAATACGCGATCG | |
| | CTGTTAAAAGGACAATTACAAACAGGAATCGAATGCAACCGGCGCAGGAACACTGCCAG | |
| | CGCATCAACAATATTTTCACCTGAATCAGGATATTCTTCTAATACCTGGAATGCTGTTT | |
| | TCCCGGGGATCGCAGTGGTGAGTAACCATGCATCATCAGGAGTACGGATAAAATGCTTG | |
| | ATGGTCGGAAGAGGCATAAATTCCGTCAGCCAGTTTAGTCTGACCATCTCATCTGTAAC | |
| | ATCATTGGCAACGCTACCTTTGCCATGTTTCAGAAACAACTCTGGCGCATCGGGCTTCC | |
| | CATACAATCGATAGATTGTCGCACCTGATTGCCCGACATTATCGCGAGCCCATTTATAC | |
| | CCATATAAATCAGCATCCATGTTGGAATTTAATCGCGGCCTCGAGCAAGACGTTTCCCG | |
| | TTGAATATGGCTCAT | |
| Multi-Enzyme | CCTAGATCCTTTTAAATTAAAAATGAAGTTTTAAATCAATCTAAAGTATATATGAGTAA | 246 |
| Backbone^{b} | ACTTGGTCTGATTATTTGCCGACTACCTTGGTGATCTCGCCTTTCACGTAGTGGACAAA | |
| | TTCTTCCAACTGATCTGTGCGCGAGGCCAAGCGATCTTCTTCTTGTCCAAGATAAGCCT | |
| | GTCTAGCTTCAAGTATGACGGGCTGATACTGGGCCGGCAGGCGCTCCATTGCCCAGTCG | |
| | GCAGCGACATCCTTCGGCGCGATTTTGCCGGTTACTGCGCTGTACCAAATGCGGGACAA | |
| | CGTAAGCACTACATTTCGCTCATCGCCAGCCCAGTCGGGCGGCGAGTTCCATAGCGTTA | |
| | AGGTTTCATTTAGCGCCTCAAATAGATCCTGTTCAGGAACCGGATCAAAGAGTTCCTCC | |
| | GCCGCTGGACCTACCAAGGCAACGCTATGTTCTCTTGCTTTTGTCAGCAAGATAGCCAG | |
| | ATCAATGTCGATCGTGGCTGGCTCGAAGATACCTGCAAGAATGTCATTGCGCTGCCATT | |
| | CTCCAAATTGCAGTTCGCGCTTAGCTGGATAACGCCACGGAATGATGTCGTCGTGCACA | |
| | ACAATGGTGACTTCTACAGCGCGGAGAATCTCGCTCTCTCCAGGGGAAGCCGAAGTTTC | |
| | CAAAAGGTCGTTGATCAAAGCTCGCCGCGTTGTTTCATCAAGCCTTACGGTCACCGTAA | |
| | CCAGCAAATCAATATCACTGTGTGGCTTCAGGCCGCCATCCACTGCGGAGCCGTACAAA | |
| | TGTACGGCCAGCAACGTCGGTTCGAGATGGCGCTCGATGACGCCAACTACCTCTGATAG | |
| | TTGAGTCGATACTTCGGCGATCACCGCTTCCCTCATTTTAGCTTCCTTAGCTCCTGAAA | |
| | ATCTCGATAACTCAAAAAATACGCCCGGTAGTGATCTTATTTCATTATGGTGAAAGTTG | |
| | GAACCTCTTACGTGCCATTTCCCCGAAAAGTGCCACCTGACGTCTAAGAAACCATTATT | |
| | ATCATGACATTAACCTATAAAAATAGGCGTATCACGAGGCAGAATTTCAGATAAAAAAA | |
| | ATCCTTAGCTTTCGCTAAGGATGATTTCTGGAATTCGCGGCCGCTTCTAGAGCGCGAAG | |
| | ACTTACGAAAATCCGCTTAACGATCGTTGGCTGttttcagcaggacgcactgacctccc | |
| | tatcagtgatagagattgacatccctatcagtgatagagatactgagcacCAGGGTGTC | |
| | TCAAGGTGCGTACCTTGACTGATGAGTCCGAAAGGACGAAACACCCCTCTACAAATAAT | |
| | 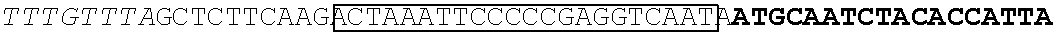 | |
| | CTGCAAATACAACCACATTTCCATGTAGAGGTCATTGAACCAAAGCAAGTCTACTTGTT | |
| | GGGTGAACAAGCTAATCATGCATTGACAGGCCAATTATACTGCCAAATTTTGCCATTGT | |
| | TAAACGGACAATACACATTGGAACAAATCGTTGAAAAACTAGACGGAGAAGTACCACCT | |
| | GAATACATTGATTATGTGCTGGAGAGACTAGCTGAGAAGGGCTATCTGACTGAAGCAGC | |
| | ACCTGAATTATCTAGTGAAGTGGCCGCTTTCTGGTCTGAGCTGGGGATTGCACCTCCTG | |
| | TCGCGGCCGAAGCATTACGTCAACCTGTGACTTTAACACCTGTTGGAAACATCAGCGAA | |
| | GTAACAGTAGCAGCCTTAACCACAGCCCTACGTGATATCGGTATTTCCGTTCAAACACC | |
| | TACAGAAGCTGGATCGCCAACTGCATTGAACGTTGTACTTACCGATGATTATCTCCAAC | |
| | CAGAACTCGCTAAGATCAATAAGCAAGCCTTAGAAAGTCAACAAACTTGGCTACTTGTC | |
| | AAACCAGTTGGCTCCGTGTTATGGTTGGGTCCGGTATTCGTGCCAGGAAAAACAGGTTG | |
| | CTGGGATTGTTTGGCTCACAGATTAAGGGGGAATAGAGAGGTAGAGGCCTCTGTATTGA | |
| | GACAAAAACAAGCTCAACAACAACGTAATGGACAAAGCGGGTCTGTAATAGGATGCCTT | |
| | CCCACGGCTAGAGCGACACTGCCCTCAACACTCCAAACTGGGCTGCAGTTCGCTGCTAC | |
| | CGAAATTGCTAAATGGATAGTTAAGTATCATGTTAATGCCACAGCGCCTGGCACCGTAT | |
| | TCTTCCCTACATTGGATGGTAAGATAATTACGCTAAATCACTCCATACTGGATTTGAAG | |
| | TCACATATTCTGATCAAGCGTTCTCAATGTCCCACCTGTGGTGACCCAAAAATCTTACA | |
| | GCACCGTGGTTTCGAACCTTTAAAACTTGAGTCAAGGCCTAAACAGTTCACCTCAGACG | |
| | GCGGACATCGTGGTACTACCCCTGAACAAACTGTCCAGAAATATCAACATTTAATCTCG | |
| | CCTGTTACCGGTGTAGTTACTGAATTGGTCAGGATAACTGATCCGGCCAATCCACTAGT | |
| | TCACACATATAGAGCTGGTCATAGCTTCGGGAGCGCTACATCGCTGAGAGGGCTGCGTA | |
| | ATACCTTAAAGCATAAGAGTTCAGGTAAGGGTAAGACTGATTCTCAAAGTAAAGCCTCG | |
| | GGCCTGTGTGAGGCGGTAGAACGTTACTCAGGAATCTTTCAAGGTGACGAACCGAGAAA | |
| | ACGCGCCACATTGGCTGAATTGGGAGATTTGGCAATTCACCCTGAGCAATGCTTGTGTT | |
| | TTTCCGACGGTCAGTACGCTAATAGAGAAACTTTAAACGAACAGGCAACGGTGGCACAT | |
| | GATTGGATACCTCAACGTTTTGATGCATCACAAGCTATTGAATGGACTCCAGTCTGGTC | |
| | CCTAACTGAACAGACCCATAAATATTTGCCCACCGCATTGTGTTACTACCATTATCCTC | |
| | TACCCCCAGAACACAGATTCGCACGTGGAGATTCGAATGGTAATGCTGCCGGAAATACG | |
| | TTGGAAGAGGCTATACTCCAAGGCTTCATGGAATTAGTCGAGAGAGATGGTGTGGCTTT | |
| | ATGGTGGTATAACAGGCTACGCAGACCCGCTGTAGACTTAGGCTCATTTAACGAGCCAT | |
| | ACTTCGTTCAGTTGCAACAATTCTACAGAGAAAACGATAGAGATTTGTGGGTTTTGGAC | |
| | TTGACAGCTGATTTAGGTATCCCGGCTTTCGCGGGCGTTTCTAATAGAAAAACTGGTAG | |
| | TTCGGAGAGGTTGATATTAGGATTCGGTGCACACCTCGATCCTACTATTGCAATTCTGA | |
| | GAGCAGTTACAGAAGTTAACCAGATTGGCCTTGAATTAGATAAAGTTCCAGACGAGAAC | |
| | CTTAAGAGCGACGCAACAGATTGGCTAATTACTGAAAAATTAGCTGACCACCCTTATTT | |
| | GTTACCAGATACAACTCAACCTCTAAAAACTGCTCAAGATTATCCTAAAAGGTGGTCTG | |
| | ACGATATATACACGGACGTAATGACTTGCGTTAATATTGCTCAACAAGCAGGACTTGAA | |
| | ACTCTAGTTATTGATCAAACACGTCCGGACATTGGTTTGAATGTTGTTAAGGTGACAGT | |
| | CCCGGGGATGAGGCACTTTTGGTCAAGATTTGGAGAGGGGAGGCTTTATGACGTGCCCG | |
| | TCAAATTAGGTTGGCTTGACGAACCTTTGACCGAAGCGCAAATGAACCCCACGCCGATG | |
| | CCTTTTTAATAATGAAGAGCTAAGCGTTGAACGCTACACGGACTCTAACTAAAAAGGCC | |
| | TCCCAAATCGGGGGGCCTTTTTTATTGATAACAAAACGGCATGCGCATGGACGACTACG | |
| | GATGCGGGCAAGGTGCCGCTTAACGATCGTTGGCTGccctttgtgcgtccaaacggacg | |
| | cacggcgctctaaagcgggtcgcgatctttcagattcgctcctcgcgctttcagtcttt | |
| | gttttggcgcatgtcgttatcgcaaaaccgctgcacacttttgcgcgacatgctctgat | |
| | ccccctcatctgggggggcctatctgagggaatttccgatccggctcgcctgaaccatt | |
| | ctgctttccacgaacttgaaaacgctCAGTCATAAGTCTGGGCTAAGCCCACTGATGAG | |
| | TCGCTGAAATGCGACGAAACTTATGACCTCTACAAATAATTTTGTTTAAGAGCCACCAG | |
| | TTATAAGGAGAACCTACCGATGACCAAAAAGTATCGGCGTGTATCCTACGCAGTGTGGG | |
| | AAATCACCCTGAAATGCAATCTGGCATGCTCTCATTGTGGCAGCCGCGCCGGCCAAGCC | |
| | CGTACGAAAGAGCTGAGTACCGAAGAAGCGTTCAACCTGGTCCGCCAGCTGGCCGACGT | |
| | GGGCATTAAGGAAGTCACCCTGATCGGTGGTGAAGCCTTTATGCGTTCGGATTGGCTGG | |
| | AAATCGCGAAAGCCGTCACTGAAGCCGGCATGATCTGTGGCATGACCACAGGGGGCTTC | |
| | GGGGTCAGTCTGGAAACGGCGCGTAAAATGAAAGAAGCGGGCATTAAAACGGTGAGCGT | |
| | TAGCATTGACGGTGGTATTCCTGAAACCCACGACCGCCAGCGCGGTAAAAAGGGTGCGT | |
| | GGCATAGTGCATTCCGGACTATGAGCCATCTGAAAGAAGTCGGGATCTACTTCGGTTGC | |
| | AACACTCAAATCAATCGTTTATCGGCGTCAGAATTCCCGATTATCTATGAACGTATTCG | |
| | CGATGCTGGGGCACGTGCGTGGCAAATTCAGCTGACGGTTCCGATGGGCAACGCCGCGG | |
| | ATAACGCAGATATGCTGCTGCAACCGTATGAATTGCTCGACATCTATCCGATGTTAGCC | |
| | CGCGTTGCCAAACGTGCGAAACAGGAAGGCGTGCGTATTCAGGCAGGTAACAACATCGG | |
| | GTACTATGGACCGTATGAGCGTCTGCTGCGTGGCAGCGACGAATGGACGTTTTGGCAAG | |
| | GATGTGGTGCGGGCCTTAACACCCTCGGCATCGAAGCCGACGGCAAAATCAAAGGCTGT | |
| | CCATCCCTGCCGACCGCCGCGTACACCGGCGGTAACATTCGCGATCGCCCGCTGCGGGA | |
| | AATCGTCGAACAGACCGAAGAACTGAAATTTAACTTAAAAGCTGGTACAGAACAAGGTA | |
| | CGGACCATATGTGGGGCTTTTGTAAAACCTGCGAATTCGCGGAACTCTGTCGCGGCGGA | |
| | TGCAGCTGGACTGCGCATGTGTTCTTTGACCGGCGCGGCAATAATCCGTACTGCCACCA | |
| | TCGGGCTCTGAAACAAGCCCAAAAAGACATTCGCGAACGCTTTTATTTAAAAGTGAAAG | |
| | CAAAGGGCAACCCGTTCGACAATGGTGAATTTGTTATCATTGAAGAACCTTTTAACGCT | |
| | CCGTTACCCGAGAATGACCTGCTGCACTTTAACAGTGATCACATTCAATGGCCAGAAAA | |
| | CTGGCAAAATAGTGAAAGCGCGTACGCATTGGCCAAGTAATAAATATAAAGTTAAGGAG | |
| | TTGCACATGAACAGTAATCAGATCCCTAACAAAGTTGCAACCGCGGCACAGAAATCTGA | |
| | CGACAGCAGCAGCGTATTACCGCGCCAGGGGTGGCAAGACAAACAAGCCTTTATTAAGG | |
| | CACTCATTAAAGCCAAACAGTCTCTCGAAATTGCCGAAATTAGCAACTTTTTAACCTAA | |
| | TAAAGAATTACCTACCGCGGTCGCTCGGTACCAAATTTTCGAAAAAAGACGCTGAAAAG | |
| | CGTCTTTTTTCGTTTTGGTCCCACGTGGCAAGCGCTTAACGATCGTTGGCTGaacaaac | |
| | agacaatctggtctgtttgtattatggaaaatttttctgtataatagattcaacaaaca | |
| | gacaatctggtctgtttgtattatCAGCGGTCAACGCATGTGCTTTGCGTTCTGATGAG | |
| | ACAGTGATGTCGAAACCGCCTCTACAAATAATTTTGTTTAAGCTCTTCAAGAGCATTCC | |
| | ATAAGGAGAAATTTTATGACGAGAACCAACACCGGCTATCGTTATCGCGCGTTCGGCCT | |
| | GCGCATAGACTCAGATATTCCGCTGCCAGAATTAGGGGACGGTACGCGCCCTGATGGTG | |
| | ACGCGGATCTGACGGTCGTCCGGTGTGGGGAAGCGGAGCCGGAATGGGCTGAAGGTGGT | |
| | GGCGGGGGTCGTCTGTATGCCGCTGAAGGCATTGTATCTTTTCGCGTGCCGCAGACGGC | |
| | AGCGTTCCGTATTACTAATGGAAATCGCATCGAGGTGCATGCCTACTCGGGGGCTGATG | |
| | AGGATCGAATACGCCTGTACGTGTTAGGGACCTGTATGGGAGCGCTGTTACTGCAACGT | |
| | AGAATCTTACCGCTTCATGGTTCGGTCGTCGCCCGTGATGGTCGTGCGTATGCCATAGT | |
| | TGGCGAAAGCGGAGCGGGCAAATCCACGATGAGTGCAGCACTTCTCGAACGTGGATTCC | |
| | GCCTCGTTACGGATGACGTGGCCGCCATCGTGTTCGATGAGCGTGGGACCCCACTGGTT | |
| | ATGCCGGCTTATCCACAGCAAAAACTGTGGCAGGATTCCCTGGACCGTCTGCAAATTGC | |
| | GGGCTCGGGCCTTCGTCCGCTGTTCGAACGCGAAACGAAATACGCTGTACCCGCGGATG | |
| | GGGCATTCTGGCCCGAACCGGTTCCATTGGTGCACATTTACGAACTGGTTCATAGCGAT | |
| | GGTCAAACGCCTGAACTGCAGCCGATTGCCAAATTAGAGCGTTGCTATACCTTGTATCG | |
| | CCACACATTTCGTAGAAGCCTGATCGTCCCCAGCGGCTTAAGCGCCTGGCATTTTGAAA | |
| | CGGCAGTGAAACTTGCGGAGAAAACGGGGATGTACCGTCTTATGCGCCCGGCCAAAGTT | |
| | TTCGCGGCTCGCGAATCTGCTCGGCTGATTGAAACTCACGCCGATGGTGAAGTGTCACG | |
| | TTAATAATGAAGAGCGGATGAGCTCTACAAATAAGCAGAGGTGGTTGTGTTGCGAAAAA | |
| | AAAAAAAAAACACCCTAACGGGTGTTTTTTTTTTTTTGGTGTCCCCACGTGTGGCGCTT | |
| | TACAAAGTCTTCCTGTACTAGTAGCGGCCGCTGCAGTCCGGCAAAAAAGGGCAAGGTGT | |
| | CACCACCCTGCCCTTTTTCTTTAAAACCGAAAAGATTACTTCGCGTTATGCAGGCTTCC | |
| | TCGCTCACTGACTCGCTGCGCTCGGTCGTTCGGCTGCGGCGAGCGGTATCAGCTCACTC | |
| | AAAGGCGGTAATTTAATAAGATGATCTTCTTGAGATCGTTTTGGTCTGCGCGTAATCTC | |
| | TTGCTCTGAAAACGAAAAAACCGCCTTGCAGGGCGGTTTTTCGAAGGTTCTCTGAGCTA | |
| | CCAACTCTTTGAACCGAGGTAACTGGCTTGGAGGAGCGCAGTCACCAAAACTTGTCCTT | |
| | TCAGTTTAGCCTTAACCGGCGCATGACTTCAAGACTAACTCCTCTAAATCAATTACCAG | |
| | TGGCTGCTGCCAGTGGTGCTTTTGCATGTCTTTCCGGGTTGGACTCAAGACGATAGTTA | |
| | CCGGATAAGGCGCAGCGGTCGGACTGAACGGGGGGTTCGTGCATACAGTCCAGCTTGGA | |
| | GCGAACTGCCTACCCGGAACTGAGTGTCAGGCGTGGAATGAGACAAACGCGGCCATAAC | |
| | AGCGGAATGACACCGGTAAACCGAAAGGCAGGAACAGGAGAGCGCACGAGGGAGCCGCC | |
| | AGGGGGAAACGCCTGGTATCTTTATAGTCCTGTCGGGTTTCGCCACCACTGATTTGAGC | |
| | GTCAGATTTCGTGATGCTTGTCAGGGGGGCGGAGCCTATGGAAAAACGGCTTTGCCGCG | |
| | GCCCTCTCACTTCCCTGTTAAGTATCTTCCTGGCATCTTCCAGGAAATCTCCGCCCCGT | |
| | TCGTAAGCCATTTCCGCTCGCCGCAGTCGAACGACCGAGCGTAGCGAGTCAGTGAGCGA | |
| | GGAAGCGGAATATATCCTGTATCACATATTCTGCTGACGCACCGGTGCAGCCTTTTTTC | |
| | TCCTGCCACATGAAGCACTTCACTGACACCCTCATCAGTGCCAACATAGTAAGCCAGTA | |
| | TACACTCCGCTACGATTATCAAAAAGGATCTTCA | |

^{a}Text formatting correspond to sequence features/components: promoters (lowercase), 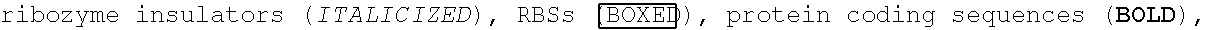
terminators (UNDERLINED), and plasmid backbone and spacers (REGULAR ALL CAPS). Each backbone plasmid sequence except for the multi-enzyme backbone includes a coding sequence for GFP. The portion of the sequence that is double underlined can be replaced with a peptide coding sequence or an RBS + enzyme coding sequence, for example chosen from Table 9, to generate a plasmid encoding a sequence of interest (e.g., a peptide or enzyme).
^{b}The multi-enzyme backbone shown is the sequence of the 6048 plasmid. To generate an alternative multi-enzyme plasmid, the protein coding sequences (shown in bold) can be replaced with coding sequences for alternative enzymes (e.g., one chosen from Table 9)

TABLE 17
Enzyme and peptide amino acid sequences

| | SEQ | |
|---|---|---|
| | ID | |
| Name | NO | Sequence |
| AlbA | 247 | MFIEQMFPFINESVRVHQLPEGGVLEIDYLRDNVSISDFEYLDLNKTAYELCMRMDGQKTA |
| enzyme | | EQILAEQCAVYDESPEDHKDWYYDMLNMLQNKQVIQLGNRASRHTITTSGSNEFPMPLHAT |
| | | FELTHRCNLKCAHCYLESSPEALGTVSIEQFKKTADMLFDNGVLTCEITGGEIFVHPNANE |
| | | ILDYVCKKFKKVAVLTNGTLMRKESLELLKTYKQKIIVGISLDSVNSEVHDSFRGRKGSFA |
| | | QTCKTIKLLSDHGIFVRVAMSVFEKNMWEIHDMAQKVRDLGAKAFSYNWVDDFGRGRDIVH |
| | | PTKDAEQHRKFMEYEQHVIDEFKDLIPIIPYERKRAANCGAGWKSIVISPFGEVRPCALFP |
| | | KEFSLGNIFHDSYESIFNSPLVHKLWQAQAPRFSEHCMKDKCPFSGYCGGCYLKGLNSNKY |
| | | HRKNICSWAKNEQLEDVVQLI |
| AlbsA | 248 | MDSLLSTETVISDDELLPIEVGGTAELTEGQGGGQSEDKRRAYNC |
| AlbsB | 249 | MPELPRFATAPRHVRALDFGHVLVLIDYRSNHVQCLLPAAAAHWTATARTGRLDTMPAALA |
| enzyme | | TQLLTSALLVPRPTATPWTAPVAAPPAPPSWGGSEHPAGTSRPRARHRHSTTAAAALACVL |
| | | AIKAAGPTRYAMQRLTTVVKAAASTCRRPATPAQATAAALAVRQACWYSPARTACLEESAA |
| | | TVILLATRRLSSTWCHGVAPDPIRLHAWVETEDGTPVAEPASTLAYTPALTIGGHHQHQP |
| AlbsC | 250 | MIFGGFSTTREVRQRPGNAEFIATDSPIWRLGRSPARCVAADHGQRRLVVLGECGATDGEL |
| enzyme | | SRLATAGLPTDITWRWPGVYVVVEEQPERTVLHTDPAAALPVYATPWQGGWAWSTSARILA |
| | | RLTEAPIDGQRLACSVLAPSVPALSGTRTFFAGIEQLALGSRIELPVDGSRLRVTVRWRPD |
| | | PVPGEPYHRLRTALTEAVALRVNRAPDLSCDLSGGLDSTSLAVLAAVCLPESHHLNAITIH |
| | | PEGDESGADLRYARLAAAHHGRIRHHLLPLAAEHLPYTEITAVPPTTEPAPSTLTRARLAW |
| | | QLDWMRQHLGSRTHMTGDGGDSVLFQPPAHLADLLRHRQWRRTLSESLGWARLRHTSVLPL |
| | | LRGAATLARTSRRSGLQDLARALAGAGQQGDGRGNVSWFAPLPLPGWATPTARRLLLDAAD |
| | | EAISTADPLPGLDTSLRVLIDEIREVARTAAADAELADAHGTTLHNPFLDPRTIDAVLRTP |
| | | IAHRPAVHSYKPALGHAMQDLLPGAVARRSTKGSFNADHYAGMRANLPALTALADGHLADL |
| | | GLLEPTRFRSHLRQAAAGIPMPLAAIEQALSAEAWCHAHHATPSPAWTTQPPEHPHA |
| AlbsT | 251 | MSTSPEQTLWISTDTCGLGPYRADLVDTYWQWEQDPTLLVGYGRQSPQSLEARTEGMAHQL |
| enzyme | | RGDNIRFTIYDLCSSTPTPAGVATLLPDHSVRTAEYVIMLAPEARGRGLGTTATQLTLDYA |
| | | FHITNLRMVWLKVLAPNTAGIRAYEKAGFRTVGALREAGYWLGKVCDEVLMDALAKDFTGP |
| | | SAVHAALTGASGRQLRRAP |
| AMdnA | 252 | MPENRQEDLNAQAVPFFARFLEGQNCEDLTDEESEAVSGGKRGQTRKYPSDCEDGNGVTGK |
| | | LRDEDIAVTLKYPSDNEDNGGGEIVTLKFPSDDDDQPVG |
| AMdnC | 253 | MNVLIITHSHDNESISLVTQAIESQGGKAFRFDTDRFPTEVQLDIYYSNTEKCVLVADDQK |
| enzyme | | LDLNEVTAVWYRRIAIGGKIPPTMDKQLRQASIQESRATIQGMIASIRGFHLDPVPNIRRA |
| | | ENKQLQLQVARKIGLDTPRTLTTNNPQAVKEFAAECQQDVITKMLSSFAIYDEKGGEQVVF |
| | | TNPVKSEDLENLEGLRFCPMTFQEKIAKVLELRITIVGKSILTAAVNSQALDKSRYDWRKQ |
| | | GVALLDAWQTHTLPQDVADKLLQLMAHFGLNYGAIDVILTPDNRYVFLEVNPVGEFFWLER |
| | | CPGLPISQAIAKVLLSHI |
| AtxA1 | 254 | MHTPIISETVQPKTAGLIVLGKASAETRGLSQGVEPDIGQTYFEESRINQD |
| AtxB | 255 | MYELNDGVGLALVDQHPIFLDLKTDRYLSLSPDGAAVLLGAAPATKESPLFLGLESIGLVK |
| enzyme | | NGPSGLKPCQIAVATGSAPPRKVQFESLSLLLLRLIRARLDQRALLKRVTDLKKAGTIAQT |
| | | KNRDCALSLLGSVETEAKACRTLLSSTDKCLPDAFAIATHLRRRGVDAKLVFGVRLPFAAH |
| | | AWVQVDDIVVGDRPDRILAFTPILVV |
| AtxC | 256 | MRYVASFFVRGHVSTPALRHPEPKGFAYAKVSGGLSVWSDAPIRHRAPLITVGAVFDRASF |
| enzyme | | KGLDCDLSGLRQDGLNTLKAETFGPYLALEVADNGTLRVYRDPSGGAPCYYLQTEDGFWLA |
| | | SDADLLFTHSGVHPSVSLPGLIEHLRRPEFQNEGTCLNVKQVRPGEQVDLSLSGEVRACLF |
| | | PPASSLRPPELHRAYDDIKAELRALILRSIKAYASDFPHVVVSFSGGLDSSVVAAGLAQTS |
| | | TKVLLHTFKGPDAKGDETAFAAECAAYLGLSLEIDTLSIDDVDLSATISPHLPRPSTSFFL |
| | | PSLLRGFSTSSQTRTGGAIFSGNGGDSVFCFMHSATPLADLMCRPSGLTPFMQTWADVQKL |
| | | TRASATEVLRRALKTAMARGYIWPESNLLLSRDTSSSRLTPDSVLSSLEGILPGRLRHLAL |
| | | IRRAHNTFEPFAPWRTPPVVHPLMAKPIQAFCLSLPSWMWVSGGKDRSLVRDAFEGLLPDS |
| | | VRLRKSKGSPAGFLHALYRAKGRQMIERIRHGYLRREGIIDISTGPDALFSEGFRNPRVMH |
| | | RFFELAATEVWIDHWRNWRRPRT |
| BamA | 257 | LKIRKVKIVRAQNGHYTN |
| BamB | 258 | MEGLYQLKVHSRIHKLQNNIAIGSMPPHALIIEDAPEYLSNVLRFFSSKKTIKEAEVYLSD |
| enzyme | | NTNLSSNEINLLLGDLIENEIIVKQNYDSNNRYSRHSLYYEMIDANAENAQKILAEKTVGL |
| | | VGMGGIGSNVAMNLAAAGVGKLIFSDGDTIELSNLTRQYLYKEDQVGLSKVESAKEQLQLL |
| | | NSEVELIPVCESISGEELFDNHFSECDFVVLSADSPFFVHEWINNAALKYGFSYSNAGYIE |
| | | TYGAIGPLVIPGETACYECYKDKGDLYLYSDNKEEFSVNLNESFQAPSYGPLNAMVSSIQA |
| | | NEVIRHLLGLKTKTSGKRLLINSEIYKIHEENFEKKNNCLCSDIKGEKLSKNTLNSDKELH |
| | | EVYIEERESDSFNSILLDKTMSKLVKINKEETKILDIGCATGEQALYFANKGAKVTAVDIS |
| | | DDMLKVLDKKASNINAGSIKTMRGNIESIEVNDTFNYIVCNNILDYLPEIDRTLRKLNMFL |
| | | KNDGTLIVTIPHPVKDGGGWRKDYYNGKWNYEEFILKDYFNEGLIEKSREDKNGETVIKSI |
| | | KTYHRTTETYFNSFTDAGFKVVSLLEPQPLSTVSETHPILFEKCSRIPYFQVFVLKKEDRH |
| | | AI |
| BmbC | 259 | MGPVVVFDCMTADFLNDDPNNAELSALEMEELESWGAWDGEATS |
| BsjA2 | 260 | MTNEEIIVAWKNPKVRGKNMPSHPSGVGFQELSINEMAQVTGGAVEQRATPTLATPLTPHT |
| | | PYATYVVSGGVVSAISGIFSNNKTCLG |
| BsjA3 | 261 | MTNEEIIVAWKNPKVRGKNMPSHPSGVGFQELSINEMAQVTGGAVEQRATPATPATPWLIK |
| | | ASYVVSGAGVSFVASYITVN |
| BsjM | 262 | MIKNVNLKEAIKGLTVSERYDTLKNSGVNLNLNISALEEWRNRKNLLADEDFTEMLTVLEY |
| enzyme | | DPVYFSHAINENIEEHIDIYKSKILGENWFIVLNDILDELDNPIEYKKEMNHSYLLRPFLL |
| | | YAEKEMNKYIVNRKELLPVEPQVIQQIMENLASKLFAVSVKSFVLELNISKLKDELAGETP |
| | | DERFHSFIRLMGEKTRLVDFYNEYIVLSRILVNITILFVNNIIELFERLQESKLDIVKKLG |
| | | VQEEFKISNISIGEGDTHQQGRSVIVLTFVSGKKVVYKPKNLKVVSAYNSLIDWINNKNNI |
| | | LKMPSYNTLIYDDFVIEEFVEKRDCKSIEEVKKYYIRYGQILGIMYILNGNDFHMENLIAS |
| | | GEYPIIVDLETLLQNIINFKNKPSADLITTKKMLNLVNSTLLLPEKLLKGDITDEGIDMSA |
| | | LAGKEQHLERREYQLKNLFTDNMVFDLEKVKIEGANNIPKLNGENVDYSTYIDEIVVGFEN |
| | | ICNLFIQYRDELLHSGILEEFKDVKVRHVLRNTVVYAKMLANTYHPDYLRDSLNREQVLEN |
| | | IWVHPFERKEFIKSEMEDILNNDIPIFFSYASSKDIIDSNGKLHKNVMEISGYERFTTKLK |
| | | ELNPFLIEQQVSVINIKTGRYGDKKFEKNYSVRDVATEKKDNPIDFLQEAMNIGDKILEHA |
| | | IICDETKTISWLTINNHHDKNWEIGPISGEFYDGLAGISLFYHYLYKKSHNVEYKKIRDYA |
| | | FNMAKVKALSLKYDSGLTGYASLLYTAHKIVQDEPRKQYKDVINEVFKYIDESKVVTAKYN |
| | | WLHGTASIIHVLLNLYEDSRDMAYLTKCIQYGKYLVKQIKEHKDMLAPGFSQGISSVIMVL |
| | | VRLSKKCEVEEFLELALELMEMERNKLGNLSESNWLNGLVGIGLSRIKLKGLDSNLQVDND |
| | | IELVLDGVMNSLYSKDDTLSCGNSGTVELFLSLFEQTKKKEYLDMAKAICGKMIEESRISF |
| | | EYQTKSLPGLELVGLYSGLAGIGYQFLRISDVEDIASIATLD |
| CapA | 263 | MVRFLAKLLRSTIHGSNGVSLDAVSSTHGTPGFQTPDARVISRFGFN |
| CapB | 264 | MQPDLEVVDVRRGESFKAWSHGYPYRTVRWHFHPEFEVHLIVETTGQMFVGDYVGGFGPGN |
| enzyme | | LVLMGPNLPHNWVSDVPEGKTVAERNLVVQFGQAFVSRCEDSLTEWRHVETLLADARRGVQ |
| | | FGPRTSEAIKPLFAELIHARGLRRIVLFLSMLQILVDATDRELLASPAYQADPSTFASTRI |
| | | NHALAYIGKNLANELRETDLARLAGQSVSAFSHYFRRHTGLPFVQYVNRMRINLACQLLMD |
| | | GDASVTDICFRSGFNNLSNFNRQFLAVKGMSPSRFRRYQALNDASRDASEAAAKRGAGIAG |
| | | APAIVPAAQARGEARPIPEVLLSG |
| CapC | 265 | MMLTASSTPASGNPAARALRAAAFALALGGACVAHAAPLRIGMTFQELNNPYFVTMQKALN |
| enzyme | | EAAASIGAQVIVTDAHHDVSKQVSDVEDMLQKKIDILLVNPTDSTGIQSAIVSAKKAGAVV |
| | | VAVDANANGPVDSFVGSKNFDAGAMSCEYLAKAINGGGEVAILDGIPVVPILERVRGCRAA |
| | | LAKFPNVKIVDVQNGKQERATALTVTENMIQAHPKLKGVFSVNDGGSMGALSAIEASGKDI |
| | | RLTSVDGAPEAVAAIQKPNSKFIETSAQFPRDQIRLAIGIGLAKKWGANVPKAIPVDVKLI |
| | | DKGNAKTFSW |
| CinA | 266 | MTASILQSVVDADFRAALIENPAAFGASTAVLPTPVEQQDQASLDFWTKDIAATEAFACKQ |
| | | SGSFGPFTFVCDGNTK |
| CinX | 267 | MALKTCEEFLRDALDPDRFGREMKAVTEIPEIVKLGHRHGYGFTAEEFLTKAMSFGAPPAG |
| enzyme | | AAAPGESASVPGQNGSSPGHAARAAMAGPEAGATSFAHYEYRLDELPEFAPVVAELPKLKV |
| | | MPPSVGPDRFAARYRDEDMRTISMSPADPAYQAWHQELAGRGWRDAEDTAAAPDAPRRDFH |
| | | LLNLDEHVDYPGYEEYFAAKTRVVAALENLFGGDVRCSGSMWYPPSSYRLWHTNADQPGWR |
| | | MYLVDVDRPFADPDRTSFFRYLHPRTREIVTLRESPRIVRFFKVEQDPEKLFWHCIANPTD |
| | | RHRWSFGYVVPENWMDALRHHG |
| Cln1A1 | 268 | MTPIQSKFCLLRVGSAKRLTQSFDVGTIKEGLVSQYYFA |
| Cln1A2 | 269 | MTQVSPSPLRLIRVGRALDLTRSIGDSGLRESMSSQTYWP |
| Cln1B | 270 | MPLWLAQDVHAVALDEDIVVLDAVSDAYLCLVGASALISLGSERSVSADPVAAETLREAGL |
| enzyme | | VGPHPSGATRPIPPKPTIDLPDAARQAQGRELRAAAWAGAATAIDFRRRSFRQLLARAGQR |
| | | PPGQAAAPADEVLAAAAVFMRLRPWSPVGGACLMRSYYLLRHLRILGFDADWIIGVRTWPF |
| | | MAHCWLQVGAVALDDDVERLTAYTPILAV |
| Cln1C | 271 | MGDYLALYWPRGMPGVAADAMRAAIEAEGAWTLAFEAYQLVVYVKGPRAPKVRALPDQGGV |
| enzyme | | VIGELFDTAATREGRVQDFPIALIKDVAAQDAARILATHAWGRYVAVLKAGDRPPWIFRDP |
| | | SGAVECLAWVRDEVTIISSDVAAQRAWSPDRLAIDWSGLGRVLARGNLWGEICPLAGVTAI |
| | | APGTARCDLGDAALSLWRPGDHARRSRHDVSPRDLARVVDASVAALARDRSAILVEISGGL |
| | | DSAIVATSLARGGAPVVAGINHYWPEPEGDERRWAQDIADRCGFRLIAGQRQRLLLDEAKL |
| | | LRHAQGPRPGLNAQDPDLDHDLAEQAKALGADALFSGQGGDGVFYQMANAALAADILMGKP |
| | | APMGRAASLAAVARRARATVWSLCGQAMFPSRAFAAGMPPPSFLSAGLAPPPVHPWIADQR |
| | | GVSPAKRIQIRGLTNIQCAFGDSLRGRAADLLYPLMAQPVMELCLSIPAPLLAVGALDRPF |
| | | ARAAFADRLPPRSLVRRSKGDVTVFFSKSLAASLPALRPFLLDGRLAEQGLIDRAKLEPLL |
| | | HPEPMIWRDSVGEVMLAAYLEAWVRAWEAKLRVS |
| Cln2A1 | 272 | MNTLKTRLIRFGSAKRLTRAGTGVLLPETNQIKRYDPA |
| Cln2A2 | 273 | MTTPKFRLIRLGSAKRLTRSGIGDVFPEPNMVRRWD |
| Cln2B | 274 | MTLTWRPGVHAVMVEDDLVLLDEAADAYVCLLDGAKVVSVRADGALSFNPPHAAEDMIAGG |
| enzyme | | LVEPSSSAAASANPPAKLPCTPLARLSRPRHVKVRPAEAALFLIQAWGVARAVRRWPMARL |
| | | LEALRGDRAAEPAKGRRSMAEACAVFDALLAWSPFDGECLFRSVLRRRFLMALGHSPDLVI |
| | | GVRTWPFRAHCWLQSGVDALDDWPERLCAYRPILAASASQGR |
| Cln2C | 275 | MSYLLMTWPPGQPSVEADALHAAFNGQGGWSLVLERFCLRVYVRGAAAPAVTLTPKGGVLI |
| enzyme | | GEMFDRAATETGAVAAYDLSRLGDDDGMAVARRVVDEAWGRYVLVLPVKERRPVVLREPLG |
| | | ALDALIWRKGDVWCVGADVPPGLEPKDLGVEETRLTHLIAEPDLASASLPLTGVAAVMPGT |
| | | AVDETGQVHRLWTPARFARSPRTDAWTAAERIPLVTRACIAALSANRSGILCEISGGLDSA |
| | | IVATSLKAEGAKISSGINFHWPQAEADERPYARAVAKSVRTRLQVVASRVAPVDPETFDEI |
| | | VVARPSFNAIDPVYDTVLAQRLIQGGEGALFTGQGGDAVFYQMPAPQLSLDLLARGPRRRG |
| | | LMGLSRRTNRSVWSLLRMGLRAPVRATFPYGARGADRPPMHPWLEDARGVGAAKRIQIEAL |
| | | VANQAVFEASRRGAAAHLVHPLLSQPLVELCLSTPAAVLAGAEQDRAFVRSAFRAQLPRLV |
| | | LDRQSKGDLSVFFAKGVARSLPGLRPRLLEGRLAARGLIDVEALSQAMQPEAMIWRDGSAE |
| | | ILCLAVLESWLRSWEARGA |
| Cln3A1 | 276 | MQRIIDETTDGLIELGAASVQTQGDVLFAPEPGVGRPPMGLSED |
| Cln3A2 | 277 | MERIEDHIDDELIDLGAASVETQGDVLNAPEPGIGREPTGLSRD |
| Cln3A3 | 278 | MEFEGIPSPDARIDLGLASEETCGQIYDHPEVGIGAYGCEGLQR |
| Cln3B | 279 | MRVAVPDHLAYCVKQGGVTFLDVRGDRYFGLPPVLEHAFVAIAEADFLLKEPNSLLEPLEA |
| enzyme | | LGVLVRGQARRADLTIPSANLSWVDEVSPTPPRLDPASLVATVTSVIRTRLSQKSKSLQAL |
| | | LEEVRTRRPGSPAHNWQLMRRLTAGFRASRAWAPIEPICLLDSLALLDFLHRRGLYPHIVF |
| | | GVIRQPFAAHCWVQADDVVLNDRLDHVGEYTPILVV |
| Cln3C | 280 | MEDYVVLIWPALAEAPARDLIRRLPKLKTVIETSGLVVLRPENGAGLRVGGNGVVLGSVFR |
| enzyme | | TGGDRETVAEFSESEASAIATSRGQQLVTEFWGGYLAVLGDASRSEVMVLRDPSGAMPAYC |
| | | LVHGEVQIICSRLEVLEDAGLGQQALNWDVVAQLLAFPNLRGRSTGLKGVEELLPGCRLTF |
| | | TGGLKTETLTWNPWLFARPSAQAPERGVAATAVRQAVEVSVRKWADQSSPVLLELSGGLDS |
| | | SIIACCLDEPRTAATFVNFVTPTAEGDERGYARLVAKAADKQLIEQDIRADEVDVTRPRPG |
| | | RHPRPASQALLQPLEQACAELAPQLGARSFFSGLGGDNVFCSIATASPAADALLTSGLGRQ |
| | | FWAAIGDLCARHNCTVWAALSATLKKLLRSDRRLVIKPNLDFLSFREDAIDRPDHPWLEVA |
| | | ADRLPGKREHVASILLAQGFLDRYEHAQVAAVRFPLLTQPVMEACLRVPTWMANHQGRNRA |
| | | VARDAFFDRLPPRVRDRQTKGGLNAFMGVAFERNRQALARHLLDGRLVQRGLIDAVAIKSA |
| | | LASPVLEGGAMNRLLYLADVESWVRSWEDV |
| ComQ | 281 | MKEIVKQNISNKDLSQLLCSFIDSKETFSFAESAILHYVVFGGENLDVATWLGAGIEILIL |
| enzyme | | SSDIMDDLEDEDNHHALWMKINRSESLNAALSLYTVGLTSIYSLNTNPLIFKYVLRYVNEA |
| | | MQGQHDDITNKSKTEDESLEVIRLKCGSLIALANVAGVLLATGEYNETVERYSYYKGIVAQ |
| | | ISGDYHVLLSGNRSDIEKNKQTLIYLYLKRLFNNASEELLYLFSHKDLYYKALLDREKFEE |
| | | KLIQAGVTQYISVLLEIYKQKCFSTIEQLNLDKEKKELIKESLLSYKKGDTRCKT |
| ComX | 282 | MQDLINYFLNYPEALKKLKNKEACLIGFDVQETETIIKAYNDYYRADPITRQWGD |
| CrnA1 | 283 | MSELSMEKVVGETFEDLSIAEMTMVQGSGDINGEFTTSPACVYSVMVVSKASSAKCAAGAS |
| | | AVSGAILSAIRC |
| CrnA2 | 284 | MSESNMKKVVGETFEDLSIAEMTKVQGSGDVMPESTPICAGFATLMSSIGLVKTIKGNVKS |
| | | FSVLI |
| CrnM | 285 | MNDINKNKTKTINEKIKIFTKEEVIDISYFEEWRSVRTLLNENYFKIMLEEMNISKNQFSY |
| enzyme | | ALQPLNDEFKLHTNVKNEEWIKCFNRVINNFNYKNINYKVGLYLPIQPFSVYLQEKLKEIL |
| | | KKLNNIKINDKIIDAFIEAHLIEMFDLVGKVIALKFEDYKQINFLKNTNNGTRLEEFLRST |
| | | FYSRKSFLKLFNEFPVLARVCTVRTIYLINNFSAIIQNINSDYLEIQEFLNVDFLNLTNIT |
| | | LSTGDSHEQGKSVSILYFDEKKLIYKPKNLKISEIFESFIDWYTNVSNHKLLDLKIPKGIF |
| | | KDDYTYNEFIEPNYCENKREIENYYNRYGYLIAICYLFNLNDLHVENVIAHGEYPVIVDIE |
| | | TSFQVPVQMEDDTLYVKLLRELELESVSSSFLLPTNLSFGMDDKVDLSALSGTMVELNQQI |
| | | LAPVNINMDNFHYEKSPSYFPGGNNIPKNNKSVTVDYKKYLLNIVTGFDEFMKYTQENQLE |
| | | FIEFLKKFSDKKIRVLVKGTEKYASMIRYSNHPNYNKEMKYRERLMMNLWAYPYKDKRIVN |
| | | SEVQDLLFNDIPIFYSFPNSRDLIDSRGLVYKDYLPVTGLQKAIDRVKDTSVKSLFDQKLI |
| | | LQSSLGLWDEILNKPVQKKELLFEKQNFNYVKEAINIAELLIGYLIETDDQSTMLSIDCSE |
| | | DKHWKIVPLDESLYGGLSGIALFFLDIYKITKDEKYFNYYDKIISTAIKQCKATIFSSSFT |
| | | GWLSPIYPLILEKKYFGTMKDKKFFDYTMEKLSNMTEEQINNMDGMDYISGKAGIVKLLIS |
| | | AYRESKNNENIGLALSKFSNDLIQNIGTGKVSELQNVGLAHGISGIMVVVASLDTFKSEYI |
| | | REQLAIEYEMFCLREDSYKWCWGISGMIQARLEILKLSPECVDKKELNLLIKRFKNILNQM |
| | | INEDSLCHGNGSIITTMKMIYMYTQDTEWNSLINLWLSNVSIYSTLQGYSIPKLGDVTIKG |
| | | LFDGICGIGWLYLYSNFSIENVLLLEV |
| CsegA1 | 286 | MTKKNATQAPRLVRVGDAHRLTQGAFVGQPEAVNPLGREIQG |
| CsegA2 | 287 | MTKTHRLIRLGDAQRLTQGTLTPGLPEDFLPGHYMPG |
| CsegA3 | 288 | MTSRFQLLRLGKADRLTRGALVGLLIEDITVARYDPM |
| CsegB | 289 | MDLWLSAGVYAVMIDDDVVFLDVATNAYFCLPAVGSVLALEGRSLRVAARELAEDLIQAGL |
| enzyme | | ASAAAAIEPPPSTRAPVRTARAVLEALPARERPRPRLAHWRQAIMAGLASRAAERRPFAQR |
| | | LPPPSTGVSPPASEGLLADLDAFRRLQPWLPFDGACLFRSQMLRDYLLALGHRVDWIFGVR |
| | | TWPFGAHCWLQAGDLVLDDEAERLIAYHPIMVR |
| CsegC | 290 | MGYAALTYPGGLAAAAFDEMVEALIDAGWTLALRAFRLAVLTDGQAPAVSPLMGRGGVAGV |
| enzyme | | LIGEAFDRRATLGGAVARAALDGLADIDPLEAGRHLIETAWGGYVGMWIGRAEAGPTLLRD |
| | | PSGALEALAWRRDGVTVMSARPLTGRAGPADLAIDWPRIVQILADPISAALGPPPLTGLAT |
| | | IDPGAAVHGADGQERSVLWTPAAVVRGARHRPWPSRQDLRRTIDATVAALASDAGPIVCEI |
| | | SGGLDSAIVATSLAASGLGPQLTVNFYGDQPEADERGYAQAVAERIGAPLRTLRREPFAFD |
| | | ETVLAAAGQAARPNFNALDPGYDAGLVGALEAIDARALFTGHGGDTVFYQVAASALAADLL |
| | | GGAPCEGSRRARLEEVARRTRRSIWSLAWEAFSGRPSTVSIEGQLLRQEAERIRRVGLTHP |
| | | WVGGLSSVTPAKRQQIRALVSNLNAHGATGRAERARIVHPLLAQPVVEACLAIPAPILSAG |
| | | EGERSFAREAFADRLPPSIVGRRSKGEISVFLNRSLAASAPFLRGFLLEGRLAARGLIDRD |
| | | ELAAALEPEAIVWKDASRDLLTAAALEAWVRHWEARIGEGEAAEGERAAGRGTAATGPRTS |
| | | ARKANTR |
| EpiA | 291 | EAVKEKNDLFNLDVKVNAKESNDSGAEPRIASKFICTPGCAKTGSFNSYCC |
| EpiD | 292 | MHGKLLICATASINVININHYIVELKQHFDEVNILFSPSSKNFINTDVLKLFCDNLYDEIK |
| enzyme | | DPLLNHINIVENHEYILVLPASANTINKIANGICDNLLTTVCLTGYQKLFIFPNMNIRMWG |
| | | NPFLQKNIDLLKSNDVKVYSPDMNKSFEISSGRYKNNITMPNIENVLNFVLNNEKRPLD |
| HalA1 | 293 | MTNLLKEWKMPLERTHNNSNPAGDIFQELEDQDILAGVNGAENLYFQGCAWYNISCRLGNK |
| | | GAYCTLTVECMPSCN |
| HalA2 | 294 | MVNSKDLRNPEFRKAQGLQFVDEVNEKELSSLAGSENLYFQGTTWPCATVGVSVALCPTTK |
| | | CTSQC |
| HalM1 | 295 | MRELQNALYFSEVVFGPNLEKIVGEKRLNFWLKLIGEDPENLKEFLSRKGNSFEEQTLPEK |
| enzyme | | EAIVPNRLGEEALEKVREELEFLNTYSTKHVRRVKELGVQIPFEGILLPFISMYIEKFQQQ |
| | | QLRKKIGPIHEEIWTQIVQDITSKLNAILHRTLILELNVARVTSQLKGDTPEERFAYYSKT |
| | | YLGKREVTHRLYSEYPVVLRLLFTTISHHISFITEILERVANDREAIETEFSPCSPIGTLA |
| | | SLHLNSGDAHHKQRTVTILEFSSSLKLVYKPRSLKVDGVFNGLLAFLNDRTGEVIKDQYCP |
| | | KVLQRDGYGYVEFVTHQSCQSLEEVSDFYERLGSLMSLSYVLNSSDFHFENIIAHGPYPVL |
| | | IDLETIIHNTADSSEETSTAMDRAFRMLNDSVLSTGMLPSSIYYRDQPNMKGLNVGGVSKS |
| | | EGQKTPFKVNQIANRNTDEMRIEKDHVTLSSQKNLPIFQSAAMESVHFLDQIQKGFTSMYQ |
| | | WIEKNKQEFKEQVRKFEGVPVRAVLRSTTRYTELLKSSYHPDLLRSALDREVLLNRLTVDS |
| | | VMTPYLKEIIPLEVEDLLNGDVPYFYTLPEERALYQEASAINSTFFTTSIFHKIDQKIDKL |
| | | GIEDHTQQMKILHMSMLASNANHYADVADLDIQKGHTIKNEQYVEMAKDIGDYLMELSVEG |
| | | ENQGEPDLCWISTVLEGSSEIIWDISPVGEDLYNGSAGVALFYAYLFKITGEKRYQEIAYK |
| | | ALVPVRRSVAQFQHHPNWSIGAFNGASGYLYAMGTIAALFNDERLKHEVTRSIPHIEPMIH |
| | | EDKIYDFIGGSAGALKVFLSLSGLFDEPKFLELAIACSEHLMKNAIKTDQGIGWKPPWEVT |
| | | PLTGFSHGVSGVMASFIELYQQTGDERLLSYIDQSLAYERSFFSEQEENWLTPNKETPVVA |
| | | WCHGAPGILVSRLLLKKCGYLDEKVEKEIEVALSTTIRKGLGNNRSLCHGDFGQLEILRFA |
| | | AEVLGDSYLQEVVNNLSGELYNLFKTEGYQSGTSRGTESVGLMVGLSGFGYGLLSAAYPSA |
| | | VPSILTLDGEIQKYREPHEA |
| HalM2 | 296 | MKTPLTSEHPSVPTTLPHTNDTDWLEQLHDILSIPVTEEIQKYFHAENDLFSFFYTPFLQF |
| enzyme | | TYQSMSDYFMTFKTDMALIERQSLLQSTLTAVHHRLFHLTHRTLISEMHIDKLTVGLNGST |
| | | PHERYMDFNHKFNKTSKSKNLFNIYPILGKLVVNETLRTINFVKKIIQHYMKDYLLLSDFF |
| | | KEKDLRLTNLQLGVGDTHVNGQCVTILTFASGQKVVYKPRSLSIDKQFGEFIEWVNSKGFQ |
| | | PSLRIPIAIDRQTYGWYEFIPHQEATSEDEIERYYSRIGGYLAIAYLFGATDLHLDNLIAC |
| | | GEHPMLIDLETLFTNDLDCYDSAFPFPALARELTQSVFGTLMLPITIASGKLLDIDLSAVG |
| | | GGKGVQSEKIKTWVIVNQKTDEMKLVEQPYVTESSQNKPTVNGKEANIGNYIPHVTDGFRK |
| | | MYRLFLNEIDELMDHNGPIFAFESCQIRHVFRATHVYAKFLEASTHPDYLQEPTRRNKLFE |
| | | SFWNITSLMAPFKKIVPHEIAELENHDIPYFVLTCGGTIVKDGYGRDIADLFQSSCIERVT |
| | | HRLQQLGSEDEARQIRYIKSSLATLTNGDWTPSHEKTPMSPASADREDGYFLREAQAIGDD |
| | | ILAQLIWEDDRHAAYLIGVSVGMNEAVTVSPLTPGIYDGTLGIVLFFDQLAQQTGETHYRH |
| | | AADALLEGMFKQLKPELMPSSAYFGLGSLFYGLMVLGLQRSDSHIIQKAYEYLKHLEECVQ |
| | | HEETPDFVSGLSGVLYMLTKIYQLTNEPRVFEVAKTTASRLSVLLDSKQPDTVLTGLSHGA |
| | | AGFALALLTYGTAANDEQLLKQGHSYLVYERNRFNKQENNWVDLRKGNAYQTFWCHGAPGI |
| | | GISRLLLAQFYDDELLHEELNAALNKTISDGFGHNHSLCHGDFGNLDLLLLYAQYTNNPEP |
| | | KELARKLAISSIDQAHTYGWKLGLNHSDQLQGMMLGVTGIGYQLLRHINPTVPSILALELP |
| | | SSTLTEKELRIHDR |
| KgpE | 297 | MKNPTLLPKLTAPVERPAVTSSDLKQASSVDAAWLNGDNNWSTPFAGVNAAWLNGDNNWST |
| | | PFAGVNAAWLNGDNNWSTPFAADGAE |
| KgpF | 298 | MINYANAQLHKSKNLMYMKAHENIFEIEALYPLELFERFMQSQTDCSIDCACKIDGDELYP |
| enzyme | | ARFSLALYNNQYAEKQIRETIDFFHQVEGRTEVKLNYQQLQHFLGADFDFSKVIRNLVGVD |
| | | ARRELADSRVKLYIWMNDYPEKMATAMAWCDDKKELSTLIVNQEFLVGFDFYFDGRTAIEL |
| | | YISLSSEEFQQTQVWERLAKVVCAPALRLVNDCQAIQIGVSRANDSKIMYYHTLNPNSFID |
| | | NLGNEMASRVHAYYRHQPVRSLVVCIPEQELTARSIQRLNMYYCMN |
| LasA | 299 | MDKRVRYEKPSLVKEGTFRKTTAGLRRLFADQLVGRRNI |
| LasB | 300 | MKGEEMLGHPQTGFVVLPDNDATGDVTGRLLPWGDVVTVYPSGRPWIIGNCWDRPVLVHDG |
| enzyme | | VIVLGHTSVTRDQIARHGNDPHRLLDEADGAFHAAVLIGHEVHVRGSAYGVCRLYTCVVDG |
| | | VTLVSDRTDVLQRLAGTDVDVDVLAGHLLEPIPHWLGEQPLLTSVEPVPPTHHVILTPDAR |
| | | SRLRPSRRRRPEPSLGLRDGAELVRERLAAAVATRVDSPALITSELSGGYDSTSVSYLAAR |
| | | GKAEVVLVTAAGRDSTSEDLWWAERAAAGLPELDHVVLPADELPFTYAGLTEPGALLDEPC |
| | | TAVAGRERVLALVRKAAARGSTLHLTGHGGDHLFTSLPTPFHDLFRTRPVAALRQLRAFGA |
| | | LAAWPTRKLMRELADRRDHSTWWRAHARPQNGQPDPHSPMLGWAIPPTVPAWVTADGVRAI |
| | | ELGILEMAERAEPLGHARGEHAELDSIFEGARMARGLNRMATHAGVPLAAPFHDDRVVEAC |
| | | LSIRPEERISAWQYKPLLNAAMQGVVPSTVLDRSAKDDGSIDVAYGLQEHRDELVALWESS |
| | | RLAETGLIDAGMLRRLCAQPSSHELEHGSLYATIACELWLRGLDQDRTQRY |
| LasC | 301 | MPVQLRRHVSFTATEYGGVLLDETKGAYWRLNTTGAEVVRAMGEAERDEIVRHVVATFDVD |
| enzyme | | AQTAAQDVDVLLAELRDAGLVAS |
| LasD | 302 | MSVNMALRGHGMSGRRRRLDATRARLAVVVARVLNLLPPRLIRRCLRVLSRGARPASIEAA |
| enzyme | | EAARRTVVAVSPAAAGAYGCLIRSIATTLVLRSRGQWPTWCVGVRAEPPFGAHAWIEAEER |
| | | LVDEPGTMHTYRRLITVGPLSRKVR |
| LasF | 303 | MSIELTPSLADLVDPLPGHALRAAATLRLADLIAAGADTAPALAAAARIDADAIARLMRYL |
| enzyme | | CSRGIFQAHEGRYALTEFSELLLDEDPSGLRKTLDQDSYGDRFDRAVAELVDVVRSGEPSY |
| | | PRLYGSTVYDDLAADPALGEVFADVRGLHSAGYGEDVAAVAGWSSCLRVVDLGGGTGSVLL |
| | | AVLERHPSLSGAVLDLPYVAPQAKKALQASAFAQRCEFIKGSFFDPLPPADRYLLCNVLFN |
| | | WDDAQAGAILARCAQAGPVAGVVVAERLIDPDAEVELVAAQDLRLLAVCGGRQRGTAEFEA |
| | | LGAAHGLALTSVTLTASGMSLLRFDVCRAGSAGGEVVEKS |
| LcnA | 304 | MTKGLDKMLLTKKKKDSMGLLNEIDVTTLDEQLGGKMSKAWCRSMVVSCVYNLVDFSSSSD |
| | | GKKTCALYRKYC |
| LcnG | 305 | MDGTNKRLEDKWFDINFLEMYTRSCLKTFGYFDEILIVKKRIEVLKNVLEKQYLSTNDYAE |
| enzyme | | EFFELNTTLESIKEYIKLNLVIEKEP1SICIMVKNEERCIKRCIDSVEILAEEIIIIDTGS |
| | | TDNTINIIEECANDKIKVFSKEWRNDFSEIRNYAIEKASSEWLVFIDADEYLDEASVLNLL |
| | | STLNIFNNHKLKDSIVLCPMINEANNTIHFRTGKFFRKDSGIKFFGTCHEEPRIKGMPNST |
| | | LLIPIKVDYLHDGYLAKVQSNKDKKTRNIELLEGMVELEPDNPRWAYMFVRDGFAILDNEY |
| | | IEKTCLRFLLLDKNVRICVNNLQDHKFTLSLLTILGRLYLRECEFEKSNLIIRILDELIPN |
| | | SLDGKFLAFMERFSKLKIEINTLLTEVIEYRRNHEVDETSLINTQGYHIDYVLSILLFETG |
| | | NYAQSKKYFDFLQENHFLEELFQDSSYSIILKMLESVED |
| LtnA1 | 306 | MNKNEIETQPVTWLEEVSDQNFDEDVFGACSTNTFSLSDYWGNNGAWCTLTHECMAWCK |
| LtnA2 | 307 | MKEKNMKKNDTIELQLGKYLEDDMIELAEGDESHGGTTPATPAISILSAYISTNTCPTTKC |
| | | TRAC |
| LtnM1 | 308 | MKFNKNVFPEINETDFDNNIKPLLDELESRITIPQEELSFSSINDDLFRELTRNEEYPYQS |
| enzyme | | ICTIVANIVMDDGSEIWRKDIFVDSNSVREAVCDILSQTLFLYFIRCFSEQIKDIRKTDED |
| | | KESTYNRYINLLFSSNFKIFSDEYPVLWYRTIRIIKNRWYSIKKSLLLTQKHRVEIDKQLD |
| | | IPHKMKIKGLKIGGDTHNGGATVTTIFFEKGYKLIYKPRSTSGEFSYKKFIEKINPYLKKD |
| | | MGAIKAIDFGEYGFSEYIECNTDEEDMKQVGQLAFFMYLLNASDMHYSNVIWTKQGPVPID |
| | | LETLFQPDRIRKGLKQSETNAYHKMEKSVYGTGIIPISLSVKGKKGEVDVGFSGIRDERSS |
| | | SPFRVLEILDGFSSDIKIVWKKQQKSSSSKNNLIVDHKKEREILQRAQSVVEGFQETSKIF |
| | | MKHREEFISIILDSFENIKIRYIHNMTFRYEQLLRTLTDAEPAQKIELDRLLLSRTGILSI |
| | | SSSPYISLSECQQMWQGDVPYFYSKFSSKSIFDTNGFVDEIELTPRQAFIIKAESITNDEV |
| | | DFQSKIIKLAFMARLSDPHTTNDNKLNKKVIIESNQQSNSSESGNKAILFLSDLLKNNVLE |
| | | DRYSHLPKTWIGPVARDGGLGWAPGVLGYDLYSGRTGPALALAAAGRVLKDKDSIELSADI |
| | | FNKSSQILQEKTYDFRNLFASGIGGFSGITGLFWALNAAGNILNNDDWIKTSNQSMLLLNE |
| | | NMLKVDKNFFDLISGNSGAIGMMYLTNPNFYLSRSKINDILLTTDCLITEMEKDETSGLAH |
| | | GVSQILWFLSIMMQRQPSSEIKIRATIVDNIIKKKYTNSYGEIECYYPTDGHSKSTSWCNG |
| | | TSGILVAYIEGYKANIVDKSSVYHIINQINVEQLQHDNIPIMCHGSLGVYESLKYASKYFE |
| | | IETKYLLDVMRNGGCSSQEVLKYYGKGNGRYPLSPGLMAGQSGALLHCCKLEDNDISVSPI |
| | | SLMT |
| LtM2 | 309 | MDPSIKKLVDSIIEFYKKDIYLAYKELEREIKNIDKTIYNTSNDEILRIFKESLISIITDD |
| enzyme | | IYRLSIKTFIYEFHKFRIDNGFPAVKDSESAFNYYISTFDVKTIARWFEKFPMLESIISSS |
| | | IKNDCTFMVDVCVNFILDLSECEKINLISEDSRLITISSSNSDPHNGGTRVLFFRFHNGDT |
| | | ILYKPRSLTVDKLISNIFEEVFEFDATNSKNPIPKVLDRGTYGWQEFIEKKSISSSEIKQA |
| | | YYNLGIFSSIFTVLGSTDIHDENLIFKGTTPYFIDLETALSPRIRYEGNEENLFYRMSSSL |
| | | FTSIVGTTIIPAKLAVHSQEIMIGAINTPAKQKTKKDGFNIINFGTDAVDIAKQNIEVERI |
| | | ANPMRIKNNIVNDPLPYQNIFTRGFKEGIKSIILKKGSIISILNNFNSPIRYIMRPTAKYY |
| | | LILDAAVFPENLYSEQTLNKTLNYLKPPKIVENSLISKQLFLAEKRILSEGDIPSFYVLGK |
| | | EKNIRAQNFISEQIFEETAVDNAIQILESISQDWVNFNERLIAEGFSYIREQSRGYLSSDF |
| | | ENSDIFKSSLTETKKSGYTAMLKTIISMSVKTSENKKIGWLPGIYDDYPISYMSAAFCSFH |
| | | DSGGIITLLEHHFGHCSPEYNEMKRGLLELGKMLKINNSNLSIISGSESLEFLYTHREVEC |
| | | LELEYILNNSAEIMGDVFLGKLGLYLILASYLKTDLKIFQDFSIICQKNLEFKKFGIAHGE |
| | | LGYLWTIFRIQNKLKNKNACLSIYHEVLNIYKGKRIESVGWCNGLSGILMILSEMSTVLEK |
| | | NQDYLFKLANLSTKLNEESVDLSVCHGASGVLQTLLFVYSNTNDKRYLSLANKYWKKVLDN |
| | | SIKYGFYNGERDKDYLLGYFQGWSGFTDSALLLDKYNNNEQVWIPINLSSDIYQHNLNNCK |
| | | EKNYEGDGCHKS |
| LynD | 310 | MQSTPLLQIQPHFHVEVIEPKQVYLLGEQANHALTGQLYCQILPLLNGQYTLEQIVEKLDG |
| enzyme | | EVPPEYIDYVLERLAEKGYLTEAAPELSSEVAAFWSELGIAPPVAAEALRQPVTLTPVGNI |
| | | SEVTVAALTTALRDIGISVQTPTEAGSPTALNVVLTDDYLQPELAKINKQALESQQTWLLV |
| | | KPVGSVLWLGPVFVPGKTGCWDCLAHRLRGNREVEASVLRQKQAQQQRNGQSGSVIGCLPT |
| | | ARATLPSTLQTGLQFAATEIAKWIVKYHVNATAPGTVFFPTLDGKIITLNHSILDLKSHIL |
| | | IKRSQCPTCGDPKILQHRGFEPLKLESRPKQFTSDGGHRGTTPEQTVQKYQHLISPVTGVV |
| | | TELVRITDPANPLVHTYRAGHSFGSATSLRGLRNTLKHKSSGKGKTDSQSKASGLCEAVER |
| | | YSGIFQGDEPRKRATLAELGDLAIHPEQCLCFSDGQYANRETLNEQATVAHDWIPQRFDAS |
| | | QAIEWTPVWSLTEQTHKYLPTALCYYHYPLPPEHRFARGDSNGNAAGNTLEEAILQGFMEL |
| | | VERDGVALWWYNRLRRPAVDLGSFNEPYFVQLQQFYRENDRDLWVLDLTADLGIPAFAGVS |
| | | NRKTGSSERLILGFGAHLDPTIAILRAVTEVNQIGLELDKVPDENLKSDATDWLITEKLAD |
| | | HPYLLPDTTQPLKTAQDYPKRWSDDIYTDVMTCVNIAQQAGLETLVIDQTRPDIGLNVVKV |
| | | TVPGMRHFWSRFGEGRLYDVPVKLGWLDEPLTEAQMNPTPMPF |
| McbA | 311 | MELKASEFGVVLSVDALKLSRQSPLGVGIGGGGGGGGGGGSCGGQGGGCGGCSNGCSGGNG |
| | | GSGGSGSHI |
| McbC | 312 | MSKHELSLVEVTHYTDPEVLAIVKDFHVRGNFASLPEFAERTFVSAVPLAHLEKFENKEVL |
| enzyme | | FRPGFSSVINISSSHNFSRERLPSGINFCDKNKLSIRTIEKLLVNAFSSPDPGSVRRPYPS |
| | | GGALYPIEVFLCRLSENTENWQAGTNVYHYLPLSQALEPVATCNTQSLYRSLSGGDSERLG |
| | | KPHFALVYCIIFEKALFKYRYRGYRMALMETGSMYQNAVLVADQIGLKNRVWAGYTDSYVA |
| | | KTMNLDQRTVAPLIVQFFGDVNDDKCLQ |
| McbD | 313 | MINVYSNLMSAWPATMAMSPKLNRNMPTFSQIWDYERITPASAAGETLKSIQGAIGEYFER |
| enzyme | | RHFFNEIVTGGQKTLYEMMPPSAAKAFTEAFFQISSLTRDEIITHKFKTVRAFNLFSLEQQ |
| | | EIPAVIIALDNITAADDLKFYPDRDTCGCSFHGSLNDAIEGSLCEFMERQSLLLYWLQGKA |
| | | NTEISSEIVTGINHIDEILLALRSEGDIRIFDITLPGAPGHAVLTLYGTKNKISRIKYSTG |
| | | LSYANSLKKALCKSVVELWQSYICLHNFLIGGYTDDDIIDSYQRHFMSCNKYESFTDLCEN |
| | | TVLLSDDVKLTFEENITSDTNLLNYLQQISDNIFVYYARERVSNSLVWYTKIVSPDFFLHM |
| | | NNSGAININNKIYHTGDGIKVRESKMVPFP |
| MdnA | 314 | MAYPNDQQGKALPFFARFLSVSKEESSIKSPSPEPTYGGTFKYPSDWEDY |
| MdnA* | 315 | MALPFFARFLSVSKEESSIKSPSPEPTYGGTFKYPSDWEDY |
| MdnC | 316 | MTVLIVTFSHDNESIPLVIKAIEAMGKKAFRFDTDRFPTEVKVDLYSGGQKGGIITDGEQK |
| enzyme | | LELKEVSSVWYRRMRYGLKLPDGMDSQFREASLKECRLSIRGMIASLSGFHLDPIAKVDHA |
| | | NHKQLQLQVAQQLGLLIPGTLTSNNPEAVKQFAREFEATGIVTKMLSQFAIYGDKQEEMVV |
| | | FTSPVTKEDLDNLEGLQFCPMTFQENIPKALELRITIVGEQIFTAAINSQQLDGAIYDWRK |
| | | EGRALHQQWQPYDLPKTIEKQLLELVKYFGLNYGAIDMIVTPDERYIFLEINPVGEFFWLE |
| | | LYPPYFPISQAIAEILVNS |
| MibA | 317 | MPADILETRTSETEDLLDLDLSIGVEEITAGPA |
| MibD | 318 | MTAHSDAGGDPRPPERLLLGVSGSVAALNLPAYIYAFRAAGVARLAVVLTPAAEGFLPAGA |
| enzyme | | LRPIVDAVHTEHDQGKGHVALSRWAQHLLVLPATANLLGCAASGLAPNFLATVLLAADCP1 |
| | | TFVPAMNPVMWRKPAVRRNVATLRADGHHVVDPLPGAVYEAASRSIVEGLAMPRPEALVRL |
| | | LGGGDDGSPAGPAGPVGRAEHVGAVEAVEAVEAVEAVEAAEALA |
| MibH | 319 | MARSEESNTLARLFDVLGDDAAAAREWVTEPHRLIASNERLGTAPEAPADDDPEAIRTVGV |
| enzyme | | IGGGTAGYLTALALKAKRPWLDVALVESADIPIIGVGEATVSYMVMFLHHYLGIDPAEFYQ |
| | | HVRPTWKLGIRFEWGSRPEGFVAPFDWGTGSVGLVGSLRETGNVNEATLQAMLMTEDRVPV |
| | | YRGEGGHVSLMKYLPFAYHMDNARLVRYLTELATRRGVHHVDATVAEVRLDGPDHVGDLIT |
| | | TDGRRLHYDFYVDCTGFRSLLLEKALGIPFESYASSLFTDAAITGTLAHGGHLKPYTTATT |
| | | MNAGWCWTIPTPESDHLGYVFSSAAIDPDDAAAEMARRFPGVTREALVRFRSGRHREAWRG |
| | | NVIAVGNSYAFVEPLESSGLLMIATAVQILVSLLPSSRRDPLPSNVANQALAHRWDAIRWF |
| | | LSIHYRFNGRLDTPFWKEARAETDISGIEPLLRLFSAGAPLTGRDSFARYLADGAAPLFYG |
| | | LEGVDTLLLGQEVPARLLPPRESPEQWRARAAAARSLASRGLRQSEALDAYAADPCLNAEL |
| | | LSDSDSWAGERVAVRAGLR |
| MibO | 320 | MIFGPDFHRDPYPVYRRLRDEAPCHHEPALGLYALSRYEDVLAALRQPTVFSSAARAVASS |
| enzyme | | AAGAGPYRGADTVSPERETAAEGPARSLLFLDPPEHQVLRQAVSRGFTPQAVLRLEPAVRD |
| | | IAAGLADRIPDRGGGEFVTEFAAPLAIAVILRLLGVPEADRARVSELLSASALSGAEAELR |
| | | SYWLGLSALLRDREDAGEGDGEDRGVVAALVRPDAGLRDADVAAGPAVRAPLTDEQVAAFC |
| | | ALVGQAGTESVAMALSNALVLFGRHHDQWRTLCARPDAIPAAFEEVLRYWAPTQHQGRTLT |
| | | AAVRLHGRLLPAGAHVLLLTGSAGRDERAYPDPDVFDIGRFHPDRRPSTALGFGLGAHFCL |
| | | GAALARLQARVALRELTRRFPRYRTDEERTVRSEVMNGFGHSRVPFST |
| MibS | 321 | MTTGTTVAHAVEPDGFRAVMATLPAAVAIVTAAAADGRPWGMTCSSVCSVTLTPPTLLVCL |
| enzyme | | RTASPTLAAVVSGRAFSVNLLCARAYPVAELFASAAADRFDRVRWRRPPGTGGPHLADDAR |
| | | AVLDCRLSESAEVGDHVVVFGQVRAIRRLSDEPPLMYGYRRYAPWPADRGPGAAGG |
| PaaA | 322 | MSLTNVKPLIKESHHIILADDGDICIGEIPGVSQVINDPPSWVRPALAKMDGKRTVPRIFK |
| enzyme | | ELVSEGVQIESEHLEGLVAGLAERKLLQDNSFFSKVLSGEEVERYNRQILQFSLIDADNQH |
| | | PFVYQERLKQSKVAIFGMGGWGTWCALQLAMSGIGTLRLIDGDDVELSNINRQVLYRTDDV |
| | | GKNKVDAAKDTILAYNENVHVETFFEFASPDRARLEELVGDSTFIILAWAALGYYRKDTAE |
| | | EIIHSIAKDKAIPVIELGGDPLEISVGPIYLNDGVHSGFDEVKNSVKDKYYDSNSDIRKFQ |
| | | EARLKHSFIDGDRKVNAWQSAPSLSIMAGIVTDQVVKTITGYDKPHLVGKKFILSLQDFRS |
| | | REEEIFK |
| PaaP | 323 | MIKFSTLSQRISAITEENAMYTKGQVIVLS |
| PadeA | 324 | MKKQYSKPSLEVLDVHQTMAGPGTSTPDAFQPDPDEDVHYDS |
| PadeK | 325 | MTERAAVRTDHYKAFGFRIESDFVLPELPPAGEREPLDNITVRRTDLQPLWNSSIHFYGNF |
| enzyme | | AILDHGRTVMFRVPGAAIYAVQDASSILVSPFDQAEENWVRLFILGTCIGIILLQRKIMPL |
| | | HGSAVAIDGKAYAIIGESGAGKSTLALHLVSKGYPLLSDDVIPVVMTQGSPWVVPSYPQQK |
| | | LWVDTLKHMGMDNANYTPLYERKTKFAVPVGSNFHEEPLPLASIFELVPWDAATHIAPIQG |
| | | MERFRVLFHHTYRNFLVQPLGLMEWHFKTLSSFVHQIGMYRLHRPMVGFSTLDLTSHILNI |
| | | TRQGENDQ |
| PalA | 326 | MKDLLKELMYEVDLEEMENLQGSGYSAAQCAWMALSCVNYIPGVGFGCGGYSACELYKRYC |
| PalS | 327 | MGNLRDFYQLMKDNYADSNLFKDLNLIHNISNDIQIGINCDFSEMLGELVGNYDSLNYPSI |
| enzyme | | TCGILTYNEERCIKRCLESVVNEFDEIIVLDSVSEDNTVKIIKENFNDVKVYVEPWKNDFS |
| | | FHRNKIINLATCDWIYFIDADNYYDSKNKGKAMRIAKVMDFLKIEGVVSPTVIEHDNSMSR |
| | | DTRKMFRLKDNILFSGKVHEEPVYANGEIPRNIIVDINVFHDGYNPKIINMMEKNERNITL |
| | | TKEMMKIEPNNPKWLYFYSRELYQTQRDIALVQSVLFKALELYENSSYTRYYVDTIALLCR |
| | | VLFESKNYQKLTECLNILENNTLNCSDIDYYNSALLFYNLLLRIKKISSTLKENIDMYERD |
| | | YHSFINPSHDHIKILILNMLLLLGDYQDAFKVYKEIKSIEIKDEFLVNVNKFKDNLLSFID |
| | | SINKI |
| PapA | 328 | MLKQINVIAGVKEPIRAYGCSANDACYFCDTRDNCKACDASDFCIKSDT |
| PapA_tev | 329 | LKQINVIAGVKEPIRAYENLYFQGCSANDACYFCDTRDNCKACDASDFCIKSDT |
| PapB | 330 | MANLIQDREDELIHFHPYKLFEVDSKTFFYNVVTNAIFEIDSLIIDILHSKGKNEEHVVKD |
| enzyme | | LAERYELSQVREAIQNMKEAYIIATDANISDVEKMGILDNSQRVFKLSSLTLFMVQECNLR |
| | | CTYCYGEEGEYNQKGKMTSEIARSAVDFLIQQSGEIEQLNITFFGGEPLLNFPLIQETVQY |
| | | VHEQSEIHNKKFSFSITTNGTLITPKIKNFFYKHHFAVQTSIDGDEKTHNFNRFFKGGQGS |
| | | YDLLLKRTEEMRNDRKIGARGTVTPAELDLSKSFDHLVKLGFRKIYLSPALYSLSDDHYDT |
| | | LSKEMVKLVEQFRELLEREDYVTAKKMSNVLGMLSKIHSGGPRIHFCGAGTNAAAVDVRGN |
| | | LFPCHRFVGEDECSIGNLFDEDPLSKQYNFIENSTVRNRTTCSKCWAKNLCGGGCHQENFA |
| | | ENGNVNQPVGKLCKVTKNFINATINLYLQLTQEQRSILFG |
| PapoA | 331 | SKKEWQEPTIEVLDINQTMAGKGWKQIDWVSDHDADLHNPS |
| PapoK | 332 | MHDRSANVSWTKYIAFGLRIASELNLPELILAAPEAVEDVVIRQADLTAWSGQLEQANFVM |
| enzyme | | LDERFMFQIPGTAIYAVREGKEIEVSIFSGADPDTVRLFVLGTCMGVLLMQRRILPIHGSA |
| | | VVIGGRAYAFVGESGTGKSTLAAAFRQAGYQMVSDDVIAVKATASSAIVYPAYPQQKLGLD |
| | | SLLQLEALRENKHARKRNNIRSLTDGNSVMPQYSDLRMLAGELNKYAVPAVDEFFNDPLPL |
| | | GGVFELVADSPIRALMREGELVAVTEQPLNVLECLHTLLQHTYRRVIIPRMGLSEWSFDTA |
| | | ARMARKVEGWRLLRDSSVFTASEVVQRVLDIIRKEEKSYGSH |
| PbtA | 333 | MNLNDLPMDVFEMADSGMEVESLTAGHGMPEVGASCNCVCGFCCSCSPSA |
| PbtM1 | 334 | MLSSALEVDIDEAAVAADLRELAAALDRSGYGEILTCFLPQKAQAHIWAQTAAKIDGPLRT |
| enzyme | | LMELFLLGRAVPQDDLPPRIAAVIPGLVSAGLVKTGQGAVWLPNLILLRPMGQWLWCQRPH |
| | | PSPTMYFGDDSLALVHRMVTYRGGRALDLCAGPGVQALTAALRSEHVTAVEINPVAAALCR |
| | | TNIAMNGLSDRMEVRLGSLYDVVRGEVFDDIVSNPPLLPVPEDVQFAFVGDGGRDGFDISW |
| | | TILDGLPEHLSDRGACRIVGCVLSDGYVPVVMEGLGEWAAKHDFDVLLTVTAHVEAHKDSS |
| | | FLRSMSLMSSAISGRPAEELQERYAADYAELGGSHVAFYELCARRGGGSARLADVSATKRS |
| | | AEVWFV |
| PbtO | 335 | MTQYPLSRPEPLGVHPDYRRLRETCPVARVGSPYGPAWLVTRYADVAAVLTDARFSRAAAP |
| enzyme | | EDDGGILLNTDPPEHDRLRKLIVAHTGTARVERLRPRAEEIAVALARRIPGEGEFISAFAE |
| | | PFSHRVLSLFVGHLVGLPAQDLGPLATVVTLAPVPDRERGAAFAELCRRLGRQVDRETLAV |
| | | VLNVVFGGHAAVVAALGYCLLAALDAPLPRLAGDPEGIAELVEETLRLAPPGDRTLLRRTT |
| | | EPVELGGRTLPAGALVIPSIAAANRDPDRPVGRRMPRHLAFGRGAHACLGMALARMELQAA |
| | | LKALAEHAPDVRLPAGTGALVRTHEELSVSPLAGIPIQR |
| PcpA | 336 | MSSNILEKVKEFFVRLVKDDAFQSQLQNNSIDEVRNILQEAGYIFSKEEFETATIELLDLK |
| | | ERDEFHELTEEELVTAVGGVTGGSGIYGPIQAMYGAVVGDPKPGKDWGWRFPSPLPKPSPI |
| | | PSPWKPPVDVQPMYGVVVSNDS |
| PcpX | 337 | MTYRRTSYAVWEITLKCNLACSHCGSRAGHTRAKELSTQEALDLVRQMADVGIIEVTLIGG |
| enzyme | | EAFLRPDWLQIAEAITKAGMLCSMTTGGYGISLETARKMKAAGIASVSVSIDGLEETHDRL |
| | | RGRKGSWQAAFKTMSHLREVGIFFGCNTQINRLSAPEFPLIYERIRDAGARAWQIQLTVPM |
| | | GRAADNANILLQPYELLDLYPMIARVARRARQEGVQIQPGNNIGYYGPYERLLRGRGSDSE |
| | | WAFWQGCAAGLSTLGIEADGAIKGCPSLPTSAYTGGNIREHSLREIVEESEQLRFNLGAGT |
| | | SQGTAHLWGFCQTCEFSELCRGGCTWTAHVFFNRRGNNPYCHHRALFQAEQGIRERVVPKV |
| | | EAQGLPFDNGEFELIEEPIDAPLPENDPLHFTSDLVQWSASWQEESESIGAVVD |
| PcpY | 338 | MVENIDNEREKSANEIEPESLLLPRQAWQSQIAYLKAILKAKQALDRIEKRYLR |
| enzyme | | |
| Pgm2 | 339 | MEREIVWTEIEESDLAAVVSASNVKDGPTVSSSNVKDR |
| PlpA1 | 340 | MSIENAKSFYERVSTDKQFRTQLENTASAEERQKIIQAAGFEFTNQEWEIAKEQILATSES |
| | | NNGELSEAELTAVSGGVDLSIFELLDEEPLFPIRPLYGLP1 |
| PlpA2 | 341 | MSIESAKAFYQRMTDDASFRTPFEAELSKEERQQLIKDSGYDFTAEEWQQAMTEIQAARSN |
| | | EELNEEELEAIAGGAVAAMYGVVFPWDNEFPWPRWGG |
| PlpX | 342 | MTKKYRRVSYAVWEITLKCNLACSHCGSRAGQARTKELSTEEAFNLVRQLADVGIKEVTLI |
| enzyme | | GGEAFMRSDWLEIAKAVTEAGMICGMTTGGFGVSLETARKMKEAGIKTVSVSIDGGIPETH |
| | | DRQRGKKGAWHSAFRTMSHLKEVGIYFGCNTQINRLSASEFPIIYERIRDAGARAWQIQLT |
| | | VPMGNAADNADMLLQPYELLDIYPMLARVAKRAKQEGVRIQAGNNIGYYGPYERLLRGSDE |
| | | WTFWQGCGAGLNTLGIEADGKIKGCPSLPTAAYTGGNIRDRPLREIVEQTEELKFNLKAGT |
| | | EQGTDHMWGFCKTCEFAELCRGGCSWTAHVFFDRRGNNPYCHHRALKQAQKDIRERFYLKV |
| | | KAKGNPFDNGEFVIIEEPFNAPLPENDLLHFNSDHIQWPENWQNSESAYALAK |
| PlpY | 343 | MNSNQIPNKVATAAQKSDDSSSVLPRQGWQDKQAFIKALIKAKQSLEIAEISNFLT |
| enzyme | | |
| ProcA* | 344 | MSEEQLKAFIAKVQADTSLQEQLKVEGADVVAIAKASGFAITTEDLNSHRQNLSDDELEGV |
| | | AGGFFCVQGTANRFTINVC |
| ProcA1.7 | 345 | MSEEQLKAFIAKVQADTSLQEQLKVEGADVVAIAKASGFAITTEDLKAHQANSQKNLSDAE |
| | | LEGVAGGTIGGTIGGTIVSITCETCDLLVGKMC |
| ProcM | 346 | MESPSSWKTSWLAAIAPDEPHKFDRRLEWDELSEENFFAALNSEPASLEEDDPCFEEALQD |
| enzyme | | ALEALKAAWDLPLLPVDNNLNRPFVDVWWPIRCHSAESLRQSFVSDSAGLADEIFDQLADS |
| | | LLDRLCALGDQVLWEAFNKERTPGTMLLAHLGAAGDGSGPPVREHYERFIQSHRRNGLAPL |
| | | LKEFPVLGRLIGTVLSLWFQGSVEMLQRICADRTVLQQCFAIPCGHHLKTVKQGLSDPHRG |
| | | GRAVAVLEFADPNSTANSSMHVVYKPKDMAVDAAYQATLADLNTHSDLSPLRTLAIHNGNG |
| | | YGYMEHVVHHLCANDKELTNFYFNAGRLTALLHLLGCTDCHHENLIACGDQLLLIDTETLL |
| | | EADLPDHISDASSTTAQPKPSSLQKQFQRSVLRSGLLPQWMFLGESKLAIDISALGMSPPN |
| | | KPERIALGWLGFNSDGMMPGRVSQPVEIPTSLPVGIGEVNPFDRFLEDFCDGFSMQSEALI |
| | | KLRNRWLDVNGVLAHFAGLPRRIVLRATRVYFTIQRQQLEPTALRSPLAQALKLEQLTRSF |
| | | LLAESKPLHWPIFAAEVKQMQHLDIPFFTHLIDADALQLGGLEQELPGFIQTSGLAAAYER |
| | | LRNLDTDEIAFQLRLIRGAVEARELHTTPESSPTLPPPATPEALMSSSAETSLEAAKRIAH |
| | | RLLELAIRDSQGQVEWLGMDLGADGESFSFGPVGLSLYGGSIGIAHLLQRLQAQQVSLMDA |
| | | DAIQTAILQPLVGLVDQPSDDGRRRWWRDQPLGLSGCGGTLLALTLQGEQAMANSLLAAAL |
| | | PRFIEADQQLDLIGGCAGLIGSLVQLGTESALQLALRAGDHLIAQQNEEGAWSSSSSQPGL |
| | | LGFSHGTAGYAAALAHLHAFSADERYRTAAAAALAYERARFNKDAGNWPDYRSIGRDSDSD |
| | | EPSFMASWCHGAPGIALGRACLWGTALWDEECTKEIGIGLQTTAAVSSVSTDHLCCGSLGL |
| | | MVLLEMLSAGPWPIDNQLRSHCQDVAFQYRLQALQRCSAEPIKLRCFGTKEGLLVLPGFFT |
| | | GLSGMGLALLEDDPSRAVVSQLISAGLWPTE |
| PsnA2 | 347 | MSKNENNKKQLRDLFIEDLGKVTGGKGGPYTTLAIGEEDPITTLAIGEEDPDPTTLALGEE |
| | | DPTTLAIGEE |
| PsnA2_ | 348 | MSKNENNKKQLRDLFIEDLGKVTGENLYFQGKGGPYTTLAIGEEDPITTLAIGEEDPDPTT |
| tev | | LALGEEDPTTLAIGEE |
| PsnB | 349 | MTNLDTSIVVVGSPDDLHVQSVTEGLRARGHEPYVFDTQRFPEEMTVSLGEQGASIFVDGQ |
| enzyme | | QIARPAAVYLRSLYQSPGAYGVDADKAMQDNWRRTLLAFRERSTLMSAVLLRWEEAGTAVY |
| | | NSPRASANITKPFQLALLRDAGLPVPRSLWTNDPEAVRRFHAEVGDCIYKPVAGGARTRKL |
| | | EAKDLEADRIERLSAAPVCFQELLTGDDVRVYVIDDQVICALRIVTDEIDFRQAEERIEAI |
| | | EISDEVKDQCVRAAKLVGLRYTGMDIKAGADGNYRVLELNASAMFRGFEGRANVDICGPLC |
| | | DALIAQTKR |
| RaxST | 350 | MDYHFISGLPRAGSSLLAALLRQNPQLHADVTSPVARLYAAMLMGMSEEHPSNVQIDDAQR |
| enzyme | | VRLLRAVFDAYYQNRQELGTVFDTNRAWCSRLTGLARLFPRSRMICCVRDVGWIVDSFERL |
| | | AQSQPLRLSALFGYDPEDSVSMHADLLTAPRGVVGYALDGLRQAFYGDHADRLLLLRYDTL |
| | | AQRPAQAMEQVYAFLQLPAFAHDYAGVQAEAERFDAALQMPGLHRVRRGVHYVPRRSVLPP |
| | | ALFDQLQELAFWESAPSHGALLV |
| RaxX | 351 | MNHSKKSPAKGAASLQRPAGAKGRPEPLDQRLWKHVGGGDYPPPGANPKHDPPPRNPGHH |
| SboA | 352 | MKKAVIVENKGCATCSIGAACLVDGPIPDFEIAGATGLFGLWG |
| SgbA | 353 | MENQDLELLARLHALPETEPVGVDGLPYGETCECVGLLTLLNTVCIGISCA |
| SgbL | 354 | MTSHATEVEWEDLLRQALHATGTGARWAVEADEMWCRVAPVPGTRREQGWKLHVSATTASA |
| enzyme | | PEVLTRALGVLLREKSGFKFARSLEQVSALNSRATPRGSSGKFITVYPRSDAEAVALARDL |
| | | HAATAGLAGPRILSDQPYAAHSLVHYRYGAFVGRRRLSDDGLLVWFIEDPDGNPVEDKRTG |
| | | RYAPPPWAVCPFPASVPVAPHDGEATSRPVVLGGRFAVREAIRQTNKGGVYRGSDTRTGTG |
| | | VVIKEARPHVEGDASGGDVRDWLRAEARTLEKLKGTGLAPEAVALFEHAGHLFLAQDEVPG |
| | | VTLRTWVAEHFRDVGGERYRADALAQVARLVDLVAAAHARGLVLRDFTPGNVMVRPDGELR |
| | | LIDLELAVLEDEAALPTHVGTPGFSAPERLADAPVRPTADYYSLGATACFVLAGKVPNLLP |
| | | EEPVGRPSEERLAAWLTACTRPLRLPDGVVDMILGLMRDDPAERWDPSRAREALRKADPTA |
| | | RPGDADRTAVRRTGSSAVAGPVPDSRTADGRTADGRSADEVVAGLVDHLVDSMTPADDRLW |
| | | PVSTLTGESDPCTVQQGAAGVLAVLTRYFELTGDPRLPGLLSTAGRWIADRTDVRSPRPGL |
| | | HFGGRGTAWALYDAGRAVDDRRLVEHALDLALAPPQATPHHDVTHGTAGSGLAALHLWQRT |
| | | GDTRFADLAVEAADRLTAAARREPSGVGWAVPAEADSPEGGKRYLGFAHGAAGIGCFLLAA |
| | | AELSRQPDHRATALEVGEGLVADAVRIGEAAQWPAQSGDLPTAPYWCHGAAGIGTFLVRLW |
| | | QATGDDRFGDLARGSAHAVAERASRAPLAQCHGLAGNGDFLLDLADATGDPVHRDTAEELA |
| | | GLILAEGTRRQGHVVFPNEYGEVSSSWSDGSAGILAFLLRTRHTGPRHWMVEQRG |
| StspA | 355 | MKKFYEAPALIERGAFAAATAGFGRLLADQLVGRLIP |
| StspM | 356 | MADHIAAGHDTVLSLAERTGTDPDLLGRVLRFLACRGVFAEPRPGTYALTPLSLTLLEGHP |
| enzyme | | SGLREWLDASGAGARMDAAVGDLLGALRSGEPSYPRLHGRPFYEDLALHSRGPAFDGLRHT |
| | | HAESYVADLLAAYPWERVRRVVDVGGGTGVLVEALMRTHATLRTVLVDLPGAVATATARIA |
| | | AAGFGNRYTPVTGSFFDPLPAGADVYTLVNVVHNWNDERASALLRRCADAGRRDSTFVIVE |
| | | RLADDADPRAITAMDLRMFLFLGGKERTAAQIREVASAAGMAHQSTIKTPSGLHLLVFRKK |
| | | RFAARGHGRRMVT |
| TbtA | 357 | MDLNDLPMDVFELADSGVAVESLTAGHGMTEVGASCNCFCYICCSCSSA |
| TfxA | 358 | MDNKVAKNVEVKKGSIKATFKAAVLKSKTKVDIGGSRQGCVA |
| TgnA* | 359 | MYRPYIAKYVEEQTLQNSTNLVYDDITQISFINKEKNVKKINLGPDTTIVTETIENADPDE |
| | | YFL |
| TgnB | 360 | MKTILIITNTLDLTVDYIINRYNHTAKFFRLNTDRFFDYDINITNSGTSIRNRKSNLIINI |
| enzyme | | QEIHSLYYRKITLPNLDGYESKYWTLMQREMMSIVEGIAETAGNFALTRPSVLRKADNKIV |
| | | QMKLAEEIGFILPQSLITNSNQAAASFCNKNNTSIVKPLSTGRILGKNKIGIIQTNLVETH |
| | | ENIQGLELSPAYFQDYIPKDTEIRLTIVGNKLFGANIKSTNQVDWRKNDALLEYKPANIPD |
| | | KIAKMCLEMMEKLEINFAAFDFIIRNGDYIFLELNANGQWLWLEDILKFDISNTIINYLLG |
| | | EPI |
| ThcoA | 361 | MRKKEWQTPELEVLDVRLTAAGPGKAKPDAVQPDEDEIVHYS |
| ThcoK | 362 | MTRTNTGYRYRAFGLRIDSDIPLPELGDGTRPDGDADLTVVRCGEAEPEWAEGGGGGRLYA |
| enzyme | | AEGIVSFRVPQTAAFRITNGNRIEVHAYSGADEDRIRLYVLGTCMGALLLQRRILPLHGSV |
| | | VARDGRAYAIVGESGAGKSTMSAALLERGFRLVTDDVAAIVFDERGTPLVMPAYPQQKLWQ |
| | | DSLDRLQIAGSGLRPLFERETKYAVPADGAFWPEPVPLVHIYELVHSDGQTPELQPIAKLE |
| | | RCYTLYRHTFRRSLIVPSGLSAWHFETAVKLAEKTGMYRLMRPAKVFAARESARLIETHAD |
| | | GEVSR |
| TruD | 363 | MQPTALQIKPHFHVEIIEPKQVYLLGEQGNHALTGQLYCQILPFLNGEYTREQIVEKLDGQ |
| enzyme | | VPEEYIDFVLSRLVEKGYLTEVAPELSLEVAAFWSELGIAPSVVAEGLKQPVTVTTAGKGI |
| | | REGIVANLAAALEEAGIQVSDPRDPKAPKAGDSTAQLQVVLTDDYLQPELAAINKEALERQ |
| | | QPWLLVKPVGSILWLGPLFVPGETGCWHCLAQRLQGNREVEASVLQQKRALQERNGQNKNG |
| | | AVSCLPTARATLPSTLQTGLQWAATEIAKWMVKRHLNAIAPGTARFPTLAGKIFTFNQTTL |
| | | ELKAHPLSRRPQCPTCGDRETLQRRGFEPLKLESRPKHFTSDGGHRAMTPEQTVQKYQHLI |
| | | GPITGVVTELVRISDPANPLVHTYRAGHSFGSATSLRGLRNVLRHKSSGKGKTDSQSRASG |
| | | LCEAIERYSGIFQGDEPRKRATLAELGDLAIHPEQCLHFSDRQYDNRESSNERATVTHDWI |
| | | PQRFDASKAHDWTPVWSLTEQTHKYLPTALCYYRYPFPPEHRFCRSDSNGNAAGNTLEEAI |
| | | LQGFMELVERDSVCLWWYNRVSRPAVDLSSEDERYFLQLQQFYQTQNRDLWVLDLTADLGI |
| | | PAFVGVSNRKAGSSERIILGFGAHLDPTVAILRALTEVNQIGLELDKVSDESLKNDATDWL |
| | | VNATLAASPYLVADASQPLKTAKDYPRRWSDDIYTDVMTCVEIAKQAGLETLVLDQTRPDI |
| | | GLNVVKVIVPGMRFWSRFGSGRLYDVPVKLGWREQPLAEAQMNPTPMPF |
| TruE* | 364 | MNKKNILPQLGQPVIRLTAGQLSSQLAELSEEALGGVDASYAVFWPICSYDD |
| TruE | 365 | MNKKNILPQLGQPVIRLTAGQLSSQLAELSEEALGGVDASTLPVPTLCSYDGVDASTVPTL |
| | | CSYDD |

TABLE 18
Genetic Parts
Promoters

| | | SEQ |
|---|---|---|
| Name | Sequence | ID NO |
| P_{CymRC} | AACAAACAGACAATCTGGTCTGTTTGTATTATGGAAAATTTTTCTGTATAATAGATTC | 366 |
| | AACAAACAGACAATCTGGTCTGTTTGTATTAT | |
| P_{LacI} | GCGGCGCGCCATCGAATGGCGCAAAACCTTTCGCGGTATGGCATGATAGCGCCC | 367 |
| P_{LacIQ} | GCGGCGCGCCATCGAATGGTGCAAAACCTTTCGCGGTATGGCATGATAGCGCCC | 368 |
| P_{LuxB} | ACCTGTAGGATCGTACAGGTTTACGCAAGAAAATGGTTTGTTACAGTCGAATAAA | 369 |
| P_{T5LacO} | AATCATAAAAAATTTATTTGCTTTGTGAGCGGATAACAATTATAATAGATTCAATTGT | 370 |
| | GAGCGGATAACAATT | |
| P_{T7A1} | ATCCCGAAAATTTATCAAAAAGAGTATTGACTTAAAGTCTAACCTATAGGATACTTAC | 371 |
| | AGCCATCGAGAGCTGCG | |

Ribosom binding sites (RBSs)

| | | | SEQ ID |
|---|---|---|---|
| Name | Gene | Sequence | NO: |
| lac1 | LacI | GGAAGAGAGTCAATTCAGGGTGGTGAAT | 372 |
| lux1 | LuxR | GGAAGAGAGTCAATTCAGGGTGGTGAAT | 373 |
| PP_1 | peptide | ACCCAACACCACCAGCAAGCCTAAGGAGGAGAAAT | 374 |
| PP_2 | MBP-TruE* | TTCCACCATCAAAACACGGAGAGTAGCCCAC | 375 |
| ME_1 | AlbA | AGAATCAAGCAAGTCAAAGGAGTTAACCCGA | 376 |
| ME_2 | AlbsB^{b} | AGAGTTTAGGAGAAAGACATAAGGAAATATTAA | 377 |
| ME_3 | AlbsC^{b} | GGAAGCAGCCGTAAAAGGTAGGTTTTTTTT | 378 |
| ME_4 | AlbsT | AGACGCTTGAACCAGCAATAAGGAGAGTAATT | 379 |
| ME_5 | AMdnC | AGAGGCTATATAGGATAGGGGGGTCCCC | 380 |
| ME_6 | AtxB^{b} | AGAGCTGTTAGTCGCTGCCAGGAGGTCCCGT | 381 |
| ME_7 | AtxC^{b} | CTTTTAACATCCCTTCTCATAAGGAGGTTTTA | 382 |
| ME_8 | BamB | GCCCCGTCAGACACCTTCTAAGGAGGACATAT | 383 |
| ME_9 | BsjM | AGAGACGGGCGGCCACCAGGAGGAACGAGA | 384 |
| ME_10 | CapB^{b} | AGAGGCCTACAGATATTCCAGACTAACACTAAGGAGGAAAACG | 385 |
| ME_11 | CapC^{b} | TGGCTTCCGTTTTTCACCACTTGTTAAGGAGTACTTT | 386 |
| ME_12 | CinX | AGAAATTTTTCATACCGAGGGAGGAAAAT | 387 |
| ME_13 | Cln1B^{b} | AGACAGTAGTATAAAGGAGGGTTCAAGT | 388 |
| ME_14 | Cln1C^{b} | TTCAATAAATTAAGGAATTTTG | 389 |
| ME_15 | Cln2B^{b} | AGAACCACTATAAGGAACGATTT | 390 |
| ME_16 | Cln2C^{b} | CAGTATAACTAGAACAACAAGGAGTCAGATA | 391 |
| ME_17 | Cln3B^{b} | AGATCCCGATAAAGGAGGTCCTA | 392 |
| ME_18 | Cln3C^{b} | TAACATAAGGAGGGTTTCTAA | 393 |
| ME_19 | ComQ | AGAGGAACGAGAAATAAGGACACAGATAT | 394 |
| ME_20 | CrnM | AGATCACCCATACCAAGTATAACGAGAACCTCC | 395 |
| ME_21 | CsegB^{b} | AGATCACTGCAATAGTAAGGAGGTATATA | 396 |
| ME_22 | CsegC^{b} | AGCACCGAGGGGTCAATAATAAGGAGGTAAAC | 397 |
| ME_23 | EpiD | ACTGAACTATAAGGTAGGTATATT | 398 |
| ME_24 | HalM1 | CCAATCAAGGAGGTAGAAAACATA | 399 |
| ME_25 | HalM2 | TAAAACCGCTCGTAAGGAGGTCTT | 400 |
| ME_26 | KgpF | AGAACGCAGACAATTTCATAGGAGGTCCCG | 401 |
| ME_27 | LasB^{b} | AGACAATTCATAAGGAGGTTAAGGT | 402 |
| ME_28 | LasC^{b} | CCTACTACTCTGATCCCCATAAGGAGGTTTTTT | 403 |
| ME_29 | LasD^{b} | CAACCTAATCTTAGGCGAGGTCATTTTTT | 404 |
| ME_30 | LasF | AGAGCCATCAGATTTAAGGAACATAAAAA | 405 |
| ME_31 | LcnG | AGACTATCGATAATAGGAGGTAGACC | 406 |
| ME_32 | LtnM1 | AGACAATTGAAGCAGGCTAGCCAGGAGTTCCAT | 407 |
| ME_33 | LtnM2 | AGAATTCCACCCCCCACTAAGGAGGTTTTTT | 408 |
| ME_34 | LynD | CTAAATTCCCCCGAGGTCAATA | 409 |
| ME_35 | McbC | AGAGCTTCACCCTACAAGGAGGATATAGA | 410 |
| ME_36 | MdnC | AGACGCCCGCAACATTTTATTTTAAGGACGACCCA | 411 |
| ME_37 | MibD | AGATAACCCAATCCGTAAGGACACACGTCAAGGAGGCGATTT | 412 |
| ME_38 | MibH^{b} | AGAGCACATCAGACCTAAGGAAAATATAA | 413 |
| ME_39 | MibO | AGAGTTCATCAGTTTATTAGGAAAAT | 414 |
| ME_40 | MibS^{b} | ACCCTGCCATTTTTTTAGCCCAAAGAACACGGAGCATCTTT | 415 |
| ME_41 | PaaA | AGATCATTTCCAATAAGGGGGACACT | 416 |
| ME_42 | PadeK | AGACACCGAAACCTAAGGAGGGATAT | 417 |
| ME_43 | PalS | AGACCAAACAATTAGGAGGACAAAT | 418 |
| ME_44 | PapB | AGAACTAAGGAGGTTAGAGG | 419 |
| ME_45 | PapoK | TTCAATCGTTAAGGAGGTACATAA | 420 |
| ME_47 | PbtM1 | AGAGGAACGGATAAGGAGGTCAATAT | 421 |
| ME_48 | PbtO | AGACGTCACTATCAAACACACTAATACCACATAAGGAGCGAACA | 422 |
| ME_49 | PcpX^{b} | AGACACAGGGAGGTCTTTAT | 423 |
| ME_50 | PcpY^{b} | CACAAGGGGGTAGTAGT | 424 |
| ME_51 | PlpX^{b} | AGAGCCACCATTTATAAGGAGAACCTACCG | 425 |
| ME_52 | PlpY^{b} | ATATAAAGTTAAGGAGTTGCAC | 426 |
| ME_53 | ProcM | AGAAATCACATTACGCATAGGGGGAGGTAGACAC | 427 |
| ME_54 | PsnB | AGACGAATATAAGGAATAAAATA | 428 |
| ME_55 | RaxST | AGAGCCTTCCACAAACTAAGGAGCACAATT | 429 |
| ME_56 | SgbL | AGAAAAACGAGGAGGTAATAG | 430 |
| ME_57 | StspM | AGAGGCGGTATTAAGGGGGCCAGAG | 431 |
| ME_58 | TgnB | AGAAATATTACAACGAGGTAAAGGC | 432 |
| ME_59 | ThcoK | AGAGCATTCCATAAGGAGAAATTTT | 433 |
| ME_60 | TruD | AGACACACTCGAATTACTCAAAGGACCTCTAGCA | 434 |
| ME_61 | TruD | AGCCACACTCGAATTACTCAAAGGACCTCTAGCA | 435 |

Terminators

| | | | SEQ |
|---|---|---|---|
| Name | Details | Sequence | ID NO: |
| B0062 | | CAGATAAAAAAAATCCTTAGCTTTCGCTAAGGATGATTTCT | 436 |
| ECK120029600 | | TTCAGCCAAAAAACTTAAGACCGCCGGTCTTGTCCACTACCTTGCA | 438 |
| | | GTAATGCGGTGGACAGGATCGGCGGTTTTCTTTTCTCTTCTCAA | |
| AraC | Includes 2 | TTGGTAACGAATCAGACAATTGACGGCTCGAGGGAGTAGCATAGGG | 438 |
| | SNPs | TTTGCAGAATCCCTGCTTCGTCCATTTGACAGGCACATTATGCATC | |
| | | GATGATAAGCTGTCAAACATGAGCA | |
| B0053 | aka His | TCCGGCAAAAAAGGGCAAGGTGTCACCACCCTGCCCTTTTTCTTTA | 439 |
| | Operon | AAACCGAAAAGATTACTTCGCGTT | |
| | Terminator | | |
| L3S3P21 | | CCAATTATTGAAGGCCTCCCTAACGGGGGGCCTTTTTTTGTTTCTG | 440 |
| | | GTCTCCC | |
| L3S2P41 | | CTCGGTACCAAAAAAAAAAAAAAAGACGCTGAAAAGCGTCTTTTTT | 441 |
| | | TTTTTTGGTCC | |
| L3S3P41 | g →c SNP to | AAAAAAAAAAAACACCCTAACGGGTGTTTTTTTTTTTTTGGTGTCC | 442 |
| | remove BsaI | C | |
| | site. | | |
| IOT | | TTGGTAACGAATCAGACAATTGACGGCTCGAGGGAGTAGCATAGGG | 443 |
| | | TTTGCAGAATCCCTGCTTCGTCCATTTGACAGGCACATTATGCATC | |
| | | GATGATAAGCTGTCAAACATGAGCAGATCCTCTACGCCGGACGCAT | |
| | | CGTGGCCGGCATCACCGGCGCCACAGGTGCGGTTGCTGGCGCCTAT | |
| | | ATCGCCGACATCACCGATGGGGAAGATCGGGCTCGCCACTTCGGGC | |
| | | TCATGAGCAAATATTTTATCTG | |

Ribozymes

| | | | SEQ |
|---|---|---|---|
| Name | Details | Sequence | ID NO: |
| RiboJ53 | | AGCGGTCAACGCATGTGCTTTGCGTTCTGATGAGACAGTGATGTCG | 444 |
| | | AAACCGCCTCTACAAATAATTTTGTTTAA | |
| ElvJ | | AGCCCCATAGGGTGGTGTGTACCACCCCTGATGAGTCCAAAAGGAC | 445 |
| | | GAAATGGGGCCTCTACAAATAATTTTGTTTAA | |

Linkers/Tags

| | | | SEQ |
|---|---|---|---|
| Name | Details | Sequence | ID NO: |
| ATag-1 | Affinity tag | ATGTCATATTACCACCATCACCATCATCACGACTATGATATTCCCA | 446 |
| | | CAAGCGAGAACTTGTACTTTCAAGGG | |
| ATag-2 | N-terminal | ATGTCATATTACCACCATCACCATCATCACGGGTCCCTGCAG | 447 |
| | SUMO affinity | | |
| | tag | | |
| ATag-3 | C-terminal | ATGTCATATTACCACCATCACCATCATCAC | 448 |
| | sumo affinity | | |
| | tag (N- | | |
| | terminal to the | | |
| | peptide) | | |
| ATag-4 | C-terminal | TCCATTACAAGCCACCATCACCATCATCACGGT | 449 |
| | sumo affinity | | |
| | tag (C- | | |
| | terminal to | | |
| | SUMO) | | |
| Link-1 | N-terminal | CATCACCATCACCACCATGGATATGATATTAGCACAGGT | 450 |
| | SUMO linker | | |
| | v1 | | |
| Link-2 | N-terminal | TGCATGTCATATTACGACTCCATTCCCACAAGCGAGAACTTGTACT | 451 |
| | SUMO linker | TTCAAGGGTGC | |
| | v2 | | |
| Link-3 | C-terminal | CGACTGGTTCCGCGTGGTAGCTATTACGACTCCATTCCCACAAGCG | 452 |
| | sumo linker | AGAAC | |
| RST_{N}* | Concatenation | ATGTCATATTACCACCATCACCATCATCACGGGTCCCTGCAGGACT | 453 |
| | of: ATag-2, | CAGAAGTCAATCAAGAAGCTAAGCCAGAGGTCAAGCCAGAAGTCAA | |
| | SUMO, and | GCCTGAGACTCACATCAATTTAAAGGTGTCCGATGGATCTTCAGAG | |
| | Link-1 | ATCTTCTTCAAGATCAAAAAGACCACTCCTTTAAGAAGGCTGATGG | |
| | | AAGCGTTCGCTAAAAGACAGGGTAAGGAAATGGACTCCTTAAGATT | |
| | | CTTGTACGACGGTATTAGAATTCAAGCTGATCAGGCCCCTGAAGAT | |
| | | TTGGACATGGAGGATAACGATATTATTGAGGCTCACCGCGAACAGA | |
| | | TTGGAGGTCATCACCATCACCACCATGGATATGATATTAGCACAGG | |
| | | T | |
| RST_{N} | Concatenation | ATGTCATATTACCACCATCACCATCATCACGGGTCCCTGCAGGACT | 454 |
| | of: ATag-2, | CAGAAGTCAATCAAGAAGCTAAGCCAGAGGTCAAGCCAGAAGTCAA | |
| | SUMO, and | GCCTGAGACTCACATCAATTTAAAGGTGTCCGATGGATCTTCAGAG | |
| | Link-2 | ATCTTCTTCAAGATCAAAAAGACCACTCCTTTAAGAAGGCTGATGG | |
| | | AAGCGTTCGCTAAAAGACAGGGTAAGGAAATGGACTCCTTAAGATT | |
| | | CTTGTACGACGGTATTAGAATTCAAGCTGATCAGGCCCCTGAAGAT | |
| | | TTGGACATGGAGGATAACGATATTATTGAGGCTCACCGCGAACAGA | |
| | | TTGGAGGTTGCATGTCATATTACGACTCCATTCCCACAAGCGAGAA | |
| | | CTTGTACTTTCAAGGGTGC | |
| RSTc | Concatenation | ATGTCATATTACCACCATCACCATCATCAC[]CGACTGGTTCCGCG | 455 |
| | of: ATag-3, | TGGTAGCTATTACGACTCCATTCCCACAAGCGAGAACGACTCAGAA | |
| | peptide insert, | GTCAATCAAGAAGCTAAGCCAGAGGTCAAGCCAGAAGTCAAGCCTG | |
| | Link-3, | AGACTCACATCAATTTAAAGGTGTCCGATGGATCTTCAGAGATCTT | |
| | SUMO, ATag- | 4CTTCAAGATCAAAAAGACCACTCCTTTAAGAAGGCTGATGGAAGC | |
| | 4. Site for | GTTCGCTAAAAGACAGGGTAAGGAAATGGACTCCTTAAGATTCTTG | |
| | peptide | TACGACGGTATTAGAATTCAAGCTGATCAGGCCCCTGAAGATTTGG | |
| | insertion is | ACATGGAGGATAACGATATTATTGAGGCTCACCGCGAACAGATTGG | |
| | indicated by []. | AGGCTCCATTACAAGCCACCATCACCATCATCACGGT | |

Genes

| | | | SEQ |
|---|---|---|---|
| Name | Details | Sequence | ID NO: |
| SUMO | sequence from | GACTCAGAAGTCAATCAAGAAGCTAAGCCAGAGGTCAAGCCAGAAG | 456 |
| | pE-SUMO | TCAAGCCTGAGACTCACATCAATTTAAAGGTGTCCGATGGATCTTC | |
| | | AGAGATCTTCTTCAAGATCAAAAAGACCACTCCTTTAAGAAGGCTG | |
| | | ATGGAAGCGTTCGCTAAAAGACAGGGTAAGGAAATGGACTCCTTAA | |
| | | GATTCTTGTACGACGGTATTAGAATTCAAGCTGATCAGGCCCCTGA | |
| | | AGATTTGGACATGGAGGATAACGATATTATTGAGGCTCACCGCGAA | |
| | | CAGATTGGAGGT | |
| lacI | | ATGAAACCAGTAACGTTATACGATGTCGCAGAGTATGCCGGTGTCT | 457 |
| | | CTTATCAGACCGTTTCCCGCGTGGTGAACCAGGCCAGCCACGTTTC | |
| | | TGCGAAAACGCGGGAAAAAGTGGAAGCGGCGATGGCGGAGCTGAAT | |
| | | TACATTCCCAACCGCGTGGCACAACAACTGGCGGGCAAACAGTCGT | |
| | | TGCTTATTGGCGTTGCCACCTCCAGTCTGGCCCTGCACGCGCCGTC | |
| | | GCAAATTGTCGCGGCGATTAAATCTCGCGCCGATCAACTGGGTGCC | |
| | | AGCGTGGTGGTGTCGATGGTAGAACGAAGCGGCGTCGAAGCCTGTA | |
| | | AAGCGGCGGTGCACAATCTTCTCGCGCAACGCGTCAGTGGGCTGAT | |
| | | CATTAACTATCCGCTGGATGACCAGGATGCCATTGCTGTGGAAGCT | |
| | | GCCTGCACTAATGTTCCGGCGTTATTTCTTGATGTCTCTGACCAGA | |
| | | CACCCATCAACAGTATTATTTTCTCCCATGAGGACGGTACGCGACT | |
| | | GGGCGTGGAGCATCTGGTCGCATTGGGTCACCAGCAAATCGCGCTG | |
| | | TTAGCGGGCCCATTAAGTTCTGTCTCGGCGCGTCTGCGTCTGGCTG | |
| | | GCTGGCATAAATATCTCACTCGCAATCAAATTCAGCCGATAGCGGA | |
| | | ACGGGAAGGCGACTGGAGTGCCATGTCCGGTTTTCAACAAACCATG | |
| | | CAAATGCTGAATGAGGGCATCGTTCCCACTGCGATGCTGGTTGCCA | |
| | | ACGATCAGATGGCGCTGGGCGCAATGCGCGCCATTACCGAGTCCGG | |
| | | GCTGCGCGTTGGTGCGGATATCTCGGTAGTGGGATACGACGATACC | |
| | | GAAGATAGCTCATGTTATATCCCGCCGTTAACCACCATCAAACAGG | |
| | | ATTTTCGCCTGCTGGGGCAAACCAGCGTGGACCGCTTGCTGCAACT | |
| | | CTCTCAGGGCCAGGCGGTGAAGGGCAATCAGCTGTTGCCAGTCTCA | |
| | | CTGGTGAAAAGAAAAACCACCCTGGCGCCCAATACGCAAACCGCCT | |
| | | CTCCCCGCGCGTTGGCCGATTCATTAATGCAGCTGGCACGACAGGT | |
| | | TTCCCGACTGGAAAGCGGGCAG | |
| HIS₆-MBP | | ATGTCATATTACCACCATCACCATCATCACGACTATGATATTCCCA | 458 |
| | | CAAGCATGAAAATCGAAGAAGGTAAACTGGTAATCTGGATTAACGG | |
| | | CGATAAAGGCTATAACGGATTGGCTGAAGTCGGTAAGAAATTCGAG | |
| | | AAAGATACCGGAATTAAAGTCACCGTTGAGCATCCGGATAAACTGG | |
| | | AAGAGAAATTCCCACAGGTTGCGGCAACTGGCGATGGCCCTGACAT | |
| | | TATCTTCTGGGCACACGACCGCTTTGGTGGCTACGCTCAATCTGGC | |
| | | CTGTTGGCTGAAATCACCCCGGACAAAGCGTTCCAGGACAAGCTGT | |
| | | ATCCGTTTACCTGGGATGCCGTACGTTACAACGGCAAGCTGATTGC | |
| | | TTACCCGATCGCTGTTGAAGCGTTATCGCTGATTTATAACAAAGAT | |
| | | CTGCTGCCGAACCCGCCAAAAACCTGGGAAGAGATCCCGGCGCTGG | |
| | | ATAAAGAACTGAAAGCGAAAGGTAAGAGCGCGCTGATGTTCAACCT | |
| | | GCAAGAACCGTACTTCACCTGGCCGCTGATTGCTGCTGACGGGGGT | |
| | | TATGCGTTCAAGTATGAAAACGGCAAGTACGACATTAAAGACGTGG | |
| | | GCGTGGATAACGCTGGCGCGAAAGCGGGTCTGACCTTCCTGGTTGA | |
| | | CCTGATTAAAAACAAACACATGAATGCAGACACCGATTACTCCATC | |
| | | GCAGAAGCTGCCTTTAATAAAGGCGAAACAGCGATGACCATCAACG | |
| | | GCCCGTGGGCATGGTCCAACATCGACACCAGCAAAGTGAATTATGG | |
| | | TGTAACGGTACTGCCGACCTTCAAGGGTCAACCATCCAAACCGTTC | |
| | | GTTGGCGTGCTGAGCGCAGGTATTAACGCCGCCAGTCCGAACAAAG | |
| | | AGCTGGCGAAAGAGTTCCTCGAAAACTATCTGCTGACTGATGAAGG | |
| | | TCTGGAAGCGGTTAATAAAGACAAACCGCTGGGTGCCGTAGCGCTG | |
| | | AAGTCTTACGAGGAAGAGTTGGCGAAAGATCCACGTATTGCCGCCA | |
| | | CCATGGAAAACGCCCAGAAAGGTGAAATCATGCCGAACATCCCGCA | |
| | | GATGTCCGCTTTCTGGTATGCCGTGCGTACTGCGGTGATCAACGCC | |
| | | GCCAGCGGTCGTCAGACTGTCGATGAAGCCCTGAAAGACGCGCAGA | |
| | | CTCGTATCACCAAGTCGTACTACCATCACCATCACCATCACGGCGG | |
| | | TAGTGGCGAAAACCTGTATTTTCAGGGT | |
| luxR | | ATGAAAAACATAAATGCCGACGACACATACAGAATAATTAATAAAA | 459 |
| | | TTAAAGCTTGTAGAAGCAATAATGATATTAATCAATGCTTATCTGA | |
| | | TATGACTAAAATGGTACATTGTGAATATTATTTACTCGCGATCATT | |
| | | TATCCTCATTCTATGGTTAAATCTGATATTTCAATCCTAGATAATT | |
| | | ACCCTAAAAAATGGAGGCAATATTATGATGACGCTAATTTAATAAA | |
| | | ATATGATCCTATAGTAGATTATTCTAACTCCAATCATTCACCAATT | |
| | | AATTGGAATATATTTGAAAACAATGCTGTAAATAAAAAATCTCCAA | |
| | | ATGTAATTAAAGAAGCGAAAACATCAGGTCTTATCACTGGGTTTAG | |
| | | TTTCCCTATTCATACGGCTAACAATGGCTTCGGAATGCTTAGTTTT | |
| | | GCACATTCAGAAAAAGACAACTATATAGATAGTTTATTTTTACATG | |
| | | CGTGTATGAACATACCATTAATTGTTCCTTCTCTAGTTGATAATTA | |
| | | TCGAAAAATAAATATAGCAAATAATAAATCAAACAACGATTTAACC | |
| | | AAAAGAGAAAAAGAATGTTTAGCGTGGGCATGCGAAGGAAAAAGCT | |
| | | CTTGGGATATTTCAAAAATATTAGGTTGCAGTGAGCGTACTGTCAC | |
| | | TTTCCATTTAACCAATGCGCAAATGAAACTCAATACAACAAACCGC | |
| | | TGCCAAAGTATTTCTAAAGCAATTTTAACAGGAGCAATTGATTGCC | |
| | | CATACTTTAAAAATTGATAA | |
| cymR | | ATGAGCCCGAAACGTCGTACCCAGGCAGAACGTGCAATGGAAACCC | 460 |
| | | AGGGTAAACTGATTGCAGCAGCACTGGGTGTTCTGCGTGAAAAAGG | |
| | | TTATGCAGGTTTTCGTATTGCAGATGTTCCGGGTGCAGCCGGTGTT | |
| | | AGCCGTGGTGCACAGAGCCATCATTTTCCGACCAAACTGGAACTGC | |
| | | TGCTGGCAACCTTTGAATGGCTGTATGAGCAGATTACCGAACGTAG | |
| | | CCGTGCACGTCTGGCAAAACTGAAACCGGAAGATGATGTTATTCAG | |
| | | CAGATGCTGGATGATGCAGCAGATTTTTTTCTGGATGATGATTTTA | |
| | | GCATCGGCCTGGATCTGATTGTTGCAGCAGATCGTGATCCGGCACT | |
| | | GCGTGAAGGTATTCTGCGTACCGTTGAACGTAATCGTTTTGTTGTT | |
| | | GAAGATATGTGGCTGGGTGTGCTGGTGAGCCGTGGTCTGAGCCGTG | |
| | | ATGATGCCGAAGATATTCTGTGGCTGATTTTTAACAGCGTTCGTGG | |
| | | TCTGACAGTTCGTAGCCTGTGGCAGAAAGATAAAGAACGTTTTGAA | |
| | | CGTGTGCGTAATAGCACCCTGGAAATTGCACGTGAACGTTATGCAA | |
| | | AATTCAAACGT | |

Modifying Enzymes

| | | | SEQ |
|---|---|---|---|
| Name | Details | Sequence | ID NO: |
| albA | Amplified | ATGTTTATAGAGCAGATGTTTCCATTTATTAATGAAAGTGTAAGAG | 461 |
| | from genome | TTCACCAGCTTCCTGAGGGCGGCGTGTTAGAAATCGACTACTTGCG | |
| | | CGATAATGTCTCCATTTCTGACTTTGAGTATTTGGATCTCAACAAA | |
| | | ACGGCTTACGAGCTCTGCATGCGCATGGATGGCCAAAAAACAGCTG | |
| | | AGCAGATTTTAGCTGAGCAATGTGCAGTGTATGATGAATCACCGGA | |
| | | AGATCATAAAGATTGGTATTACGACATGCTCAACATGCTCCAGAAC | |
| | | AAGCAGGTTATTCAGCTTGGAAACCGGGCCAGCCGCCATACAATCA | |
| | | CCACGAGCGGAAGCAATGAATTTCCGATGCCCCTGCACGCCACCTT | |
| | | TGAACTGACGCACCGCTGTAATTTGAAATGCGCCCACTGTTATTTG | |
| | | GAAAGCTCACCTGAAGCGCTCGGCACCGTGTCGATTGAGCAATTCA | |
| | | AAAAAACGGCTGATATGCTGTTTGATAACGGTGTATTGACATGCGA | |
| | | AATCACAGGTGGAGAAATTTTTGTCCATCCAAACGCCAATGAGATT | |
| | | CTTGACTATGTGTGTAAAAAGTTCAAAAAAGTCGCTGTCTTAACAA | |
| | | ACGGAACACTCATGCGAAAAGAGAGCCTGGAGCTTTTGAAAACTTA | |
| | | CAAGCAAAAAATCATCGTCGGCATTTCTCTAGATAGTGTCAATTCC | |
| | | GAGGTCCATGACTCCTTTAGAGGGAGAAAAGGCTCTTTTGCCCAAA | |
| | | CTTGTAAAACGATAAAATTGTTGAGTGACCACGGTATATTTGTCAG | |
| | | AGTCGCTATGTCTGTATTCGAAAAAAACATGTGGGAAATCCACGAT | |
| | | ATGGCCCAAAAGGTTCGGGATCTCGGGGCGAAGGCGTTTTCTTACA | |
| | | ATTGGGTTGACGATTTCGGAAGAGGCAGGGATATTGTCCATCCAAC | |
| | | GAAAGACGCCGAGCAGCACCGCAAGTTTATGGAATACGAGCAACAT | |
| | | GTGATTGATGAGTTTAAAGATCTGATTCCGATTATTCCCTATGAGA | |
| | | GAAAACGCGCGGCAAATTGCGGCGCTGGCTGGAAGTCCATTGTGAT | |
| | | CAGTCCGTTCGGCGAAGTACGTCCTTGCGCCCTCTTTCCAAAGGAA | |
| | | TTTTCATTGGGAAATATTTTTCATGATTCCTATGAAAGCATCTTTA | |
| | | ACTCCCCTCTCGTCCATAAACTGTGGCAAGCGCAAGCGCCGCGGTT | |
| | | CAGCGAACATTGCATGAAAGACAAATGCCCGTTCAGCGGCTATTGC | |
| | | GGAGGCTGTTACTTAAAAGGGCTGAACTCTAACAAATATCACCGGA | |
| | | AAAACATTTGCTCTTGGGCGAAAAATGAACAATTAGAAGATGTGGT | |
| | | CCAGCTTATT | |
| albsB | Codon | ATGCCTGAGCTTCCCCGTTTCGCGACGGCCCCTCGTCACGTGCGTG | 462 |
| | optimized | CCCTGGATTTCGGTCATGTTCTGGTCCTGATCGATTACCGTTCCAA | |
| | | TCACGTCCAGTGCCTGCTTCCGGCAGCCGCAGCCCATTGGACAGCC | |
| | | ACAGCGCGTACCGGCCGCTTGGACACCATGCCGGCAGCGCTGGCCA | |
| | | CCCAGTTACTGACATCGGCGTTATTAGTACCGCGGCCGACCGCAAC | |
| | | ACCGTGGACGGCACCTGTAGCGGCACCACCTGCTCCACCGTCATGG | |
| | | GGTGGATCCGAGCATCCTGCCGGGACATCACGCCCTCGGGCACGTC | |
| | | ATCGGCACTCAACCACGGCTGCGGCGGCGCTGGCATGTGTGCTGGC | |
| | | GATTAAGGCAGCAGGCCCAACCCGCTATGCTATGCAGCGCTTGACC | |
| | | ACGGTCGTGAAGGCAGCCGCTTCTACGTGCCGTCGCCCGGCAACGC | |
| | | CAGCACAAGCGACGGCTGCTGCGCTTGCGGTCCGTCAGGCATGCTG | |
| | | GTACTCGCCAGCGCGTACAGCCTGTCTGGAAGAATCCGCCGCGACT | |
| | | GTCATTTTACTCGCTACCCGGCGTTTGAGTTCGACATGGTGCCATG | |
| | | GAGTAGCTCCCGATCCGATTCGCCTCCATGCCTGGGTGGAAACTGA | |
| | | GGATGGGACACCTGTAGCAGAGCCAGCCTCGACCCTTGCGTACACC | |
| | | CCGGCCTTAACCATTGGAGGCCACCATCAACACCAGCCT | |
| albsC | Codon | ATGATCTTTGGTGGATTTTCGACGACCCGTGAAGTTCGTCAACGCC | 463 |
| | optimized | CTGGTAATGCCGAGTTTATTGCTACGGACTCGCCTATTTGGCGCCT | |
| | | CGGTCGTAGTCCAGCTCGTTGCGTGGCTGCGGACCATGGACAGCGT | |
| | | CGCCTGGTAGTGTTGGGAGAATGCGGGGCAACGGATGGCGAATTAT | |
| | | CTCGCCTGGCGACCGCGGGGCTGCCCACGGATATTACCTGGCGCTG | |
| | | GCCAGGCGTGTACGTGGTGGTCGAAGAACAACCGGAACGTACGGTG | |
| | | CTGCACACTGATCCAGCAGCTGCACTCCCGGTATACGCAACCCCTT | |
| | | GGCAAGGCGGCTGGGCATGGTCAACCAGCGCGCGCATCCTGGCACG | |
| | | TTTAACAGAAGCTCCAATTGATGGTCAACGCCTGGCATGTTCAGTG | |
| | | CTGGCCCCGTCTGTTCCGGCTCTGAGCGGTACCCGCACATTCTTTG | |
| | | CGGGTATCGAACAATTGGCCCTGGGTTCGCGTATTGAACTGCCGGT | |
| | | GGATGGGTCCCGTCTGCGTGTTACGGTACGTTGGCGCCCGGATCCA | |
| | | GTCCCGGGAGAACCATATCATCGCTTGCGCACAGCGTTGACCGAGG | |
| | | CGGTCGCCCTGCGTGTCAACCGCGCACCAGACCTGTCATGCGACCT | |
| | | CTCGGGCGGCCTCGATTCCACGTCACTGGCAGTCCTGGCGGCTGTG | |
| | | TGCTTACCGGAGTCCCACCATCTGAATGCTATCACGATTCATCCGG | |
| | | AGGGCGATGAAAGTGGCGCGGACTTACGGTATGCGCGCTTGGCAGC | |
| | | TGCGCACCACGGGCGTATTCGCCACCACCTTCTCCCCCTTGCGGCA | |
| | | GAACACCTGCCGTATACTGAAATTACGGCGGTGCCCCCTACCACCG | |
| | | AACCGGCACCTTCAACATTAACGCGTGCACGCCTCGCGTGGCAGTT | |
| | | AGATTGGATGCGCCAGCACTTAGGCAGCCGCACCCATATGACTGGC | |
| | | GATGGAGGCGACAGCGTACTGTTCCAACCGCCGGCACATCTGGCGG | |
| | | ATCTCCTGCGGCATCGGCAGTGGCGTCGGACTTTGTCGGAAAGTTT | |
| | | GGGATGGGCACGCCTTCGCCATACGTCTGTTTTACCCTTACTGCGT | |
| | | GGAGCAGCAACTCTTGCACGTACATCACGTCGGTCGGGCCTCCAGG | |
| | | ATCTCGCACGCGCATTGGCGGGTGCAGGTCAGCAGGGCGATGGTCG | |
| | | TGGCAATGTGAGCTGGTTCGCACCATTACCGCTGCCTGGCTGGGCG | |
| | | ACCCCAACCGCTCGTCGCTTACTGCTTGATGCAGCCGATGAAGCTA | |
| | | TCTCGACCGCGGATCCGTTACCGGGACTGGATACGTCGCTGCGCGT | |
| | | ACTGATCGATGAAATTCGCGAAGTCGCCCGCACGGCAGCGGCAGAT | |
| | | GCCGAACTGGCGGATGCTCACGGAACGACTCTGCATAACCCATTTC | |
| | | TCGATCCGCGCACTATTGATGCAGTCCTGCGCACGCCAATCGCACA | |
| | | TCGCCCGGCGGTCCACTCGTATAAGCCAGCGCTGGGGCATGCAATG | |
| | | CAGGATTTGCTCCCGGGTGCAGTCGCTCGGCGCTCAACTAAAGGCT | |
| | | CTTTTAACGCCGATCATTATGCGGGGATGCGTGCAAATCTGCCAGC | |
| | | ATTGACAGCGCTGGCAGATGGCCACCTGGCCGACCTGGGTTTGTTG | |
| | | GAGCCGACGCGCTTCCGCAGTCATCTTCGCCAAGCCGCCGCGGGCA | |
| | | TTCCGATGCCGCTTGCGGCGATCGAACAGGCGCTGTCTGCCGAAGC | |
| | | ATGGTGTCATGCACATCACGCCACCCCAAGCCCTGCCTGGACAACG | |
| | | CAGCCACCGGAACACCCGCATGCC | |
| albsT | Codon | ATGAGCACGTCCCCCGAACAGACCCTCTGGATCTCAACTGATACCT | 464 |
| | optimized | GTGGTCTGGGGCCGTATCGCGCTGACTTGGTGGATACCTATTGGCA | |
| | | GTGGGAACAAGACCCAACATTGCTTGTAGGCTACGGTCGTCAGTCA | |
| | | CCGCAGTCACTGGAGGCCCGCACGGAAGGTATGGCCCACCAATTGC | |
| | | GTGGCGATAACATCCGTTTCACTATCTATGATCTGTGCAGCAGTAC | |
| | | ACCTACCCCGGCGGGCGTGGCAACGCTGCTGCCCGATCATAGCGTC | |
| | | CGTACTGCCGAGTATGTTATTATGCTTGCGCCTGAAGCACGTGGGC | |
| | | GTGGCTTAGGAACCACCGCCACGCAGCTGACGTTAGATTATGCGTT | |
| | | TCACATCACCAATCTGCGGATGGTCTGGTTGAAAGTACTGGCGCCG | |
| | | AACACCGCGGGCATCCGTGCGTATGAGAAAGCTGGCTTTCGTACAG | |
| | | TTGGAGCGCTTCGCGAAGCCGGCTATTGGCTGGGGAAGGTCTGCGA | |
| | | TGAGGTACTGATGGATGCCTTAGCGAAAGACTTCACGGGTCCAAGT | |
| | | GCAGTCCACGCAGCATTAACTGGCGCCAGCGGTCGCCAGCTGCGCC | |
| | | GTGCACCT | |
| amdnC | Codon | ATGAACGTTCTGATTATAACGCATTCCCACGATAACGAGAGCATTT | 465 |
| | optimized | CATTGGTAACCCAAGCCATTGAATCCCAGGGTGGTAAAGCATTTCG | |
| | | CTTCGATACCGATCGTTTTCCGACGGAAGTCCAGCTGGACATCTAT | |
| | | TACTCAAATACAGAGAAATGCGTGCTGGTGGCTGACGATCAAAAAC | |
| | | TGGATTTAAATGAAGTAACCGCGGTCTGGTATCGCCGCATTGCGAT | |
| | | CGGTGGCAAAATCCCGCCCACGATGGATAAGCAACTTCGTCAGGCC | |
| | | TCGATTCAGGAGAGTCGTGCTACAATTCAAGGCATGATAGCGAGCA | |
| | | TTCGCGGCTTTCACCTTGACCCAGTGCCGAACATTCGTCGCGCTGA | |
| | | AAATAAGCAACTGCAGCTGCAGGTTGCCCGCAAAATCGGACTGGAT | |
| | | ACCCCACGCACTCTCACCACTAATAATCCGCAGGCCGTGAAGGAAT | |
| | | TTGCGGCAGAATGCCAGCAGGACGTAATCACCAAAATGCTGAGTAG | |
| | | TTTTGCGATTTATGATGAGAAAGGCGGAGAACAGGTGGTTTTCACC | |
| | | AATCCCGTGAAATCTGAGGATCTGGAAAATTTAGAAGGTCTGCGCT | |
| | | TTTGCCCTATGACGTTTCAAGAGAAAATCGCAAAGGTTCTGGAGCT | |
| | | CCGGATCACCATCGTGGGTAAGTCAATTTTAACGGCTGCGGTGAAT | |
| | | TCACAGGCCCTGGACAAATCCCGTTATGATTGGCGCAAGCAGGGCG | |
| | | TAGCATTACTGGATGCATGGCAGACCCATACGTTACCCCAGGACGT | |
| | | GGCTGATAAATTGCTTCAACTGATGGCCCATTTCGGGTTAAACTAT | |
| | | GGAGCCATTGACGTGATTCTGACCCCGGATAATCGCTATGTGTTCT | |
| | | TGGAGGTCAATCCGGTGGGCGAATTCTTTTGGCTTGAGCGTTGCCC | |
| | | AGGTCTGCCGATTAGTCAAGCTATTGCTAAAGTGCTGCTTTCTCAT | |
| | | ATA | |
| atxB | Codon | ATGTACGAGCTGAATGATGGCGTAGGTTTGGCCCTCGTGGATCAGC | 466 |
| | optimized | ATCCGATTTTTCTGGACCTGAAAACAGACCGTTACCTGTCGTTGAG | |
| | | TCCAGATGGGGCAGCAGTCCTGCTGGGAGCAGCGCCAGCCACCAAA | |
| | | GAGAGTCCACTGTTTCTCGGATTAGAATCCATTGGCTTGGTCAAAA | |
| | | ACGGTCCGTCAGGCCTTAAGCCTTGCCAAATTGCCGTAGCCACTGG | |
| | | GTCTGCACCGCCCCGTAAGGTGCAATTCGAGTCGTTGTCACTCCTG | |
| | | CTTTTGCGCTTAATTCGTGCACGTCTGGATCAACGTGCTCTTTTGA | |
| | | AGCGTGTGACCGACTTAAAGAAGGCCGGCACCATTGCCCAGACGAA | |
| | | GAACCGTGACTGCGCCTTGTCATTATTAGGTAGCGTGGAGACTGAG | |
| | | GCAAAGGCTTGTCGTACCCTTTTAAGTAGTACAGACAAATGCCTGC | |
| | | CCGACGCATTCGCAATTGCAACGCACCTGCGCCGTCGCGGAGTAGA | |
| | | CGCCAAGTTAGTTTTCGGTGTGCGCCTGCCATTCGCGGCACATGCC | |
| | | TGGGTCCAGGTAGATGATATTGTAGTGGGTGATCGTCCCGACCGTA | |
| | | TCCTTGCGTTCACCCCCATCTTAGTCGTT | |
| atxC | Codon | ATGCGCTATGTCGCGTCTTTCTTTGTTCGCGGACATGTCAGCACAC | 467 |
| | optimized | CAGCACTGCGTCACCCAGAGCCAAAGGGTTTCGCTTATGCAAAAGT | |
| | | CAGTGGCGGACTGAGCGTATGGAGCGATGCGCCGATTCGTCACCGT | |
| | | GCGCCCCTTATTACAGTGGGCGCGGTGTTCGATCGCGCGTCTTTTA | |
| | | AAGGGCTGGATTGCGACTTATCAGGTCTGCGTCAGGATGGTCTTAA | |
| | | TACATTGAAAGCGGAAACGTTCGGACCCTACCTGGCGTTAGAGGTT | |
| | | GCCGATAACGGCACCCTTCGCGTTTATCGCGATCCGTCAGGCGGCG | |
| | | CGCCTTGCTATTACCTGCAGACCGAGGACGGCTTCTGGCTTGCAAG | |
| | | CGATGCTGATTTGTTATTCACTCATTCGGGCGTACATCCATCAGTA | |
| | | AGCTTACCGGGACTGATTGAACACTTGCGTCGTCCAGAGTTCCAAA | |
| | | ATGAGGGCACATGCTTAAACGTCAAGCAAGTACGCCCTGGGGAGCA | |
| | | GGTTGATTTATCGCTCTCGGGCGAGGTCCGTGCCTGTTTGTTCCCG | |
| | | CCTGCATCATCCCTGCGCCCGCCTGAGTTGCACCGCGCATACGATG | |
| | | ACATTAAGGCTGAGCTGCGCGCTCTGATTTTACGCAGCATTAAGGC | |
| | | CTATGCCAGTGATTTCCCTCACGTTGTTGTTAGCTTCAGCGGTGGT | |
| | | CTGGATAGCAGTGTTGTTGCGGCCGGCTTAGCGCAAACTTCCACTA | |
| | | AGGTCCTGCTTCACACCTTTAAGGGCCCAGATGCCAAAGGGGACGA | |
| | | GACTGCCTTCGCCGCAGAATGCGCGGCATATCTGGGTTTAAGCTTA | |
| | | GAGATTGATACTCTCAGTATCGATGACGTTGATCTGTCGGCAACTA | |
| | | TTTCCCCGCACCTGCCGCGCCCCAGCACATCATTCTTCTTGCCATC | |
| | | ACTGCTGCGCGGTTTCTCTACCTCGAGCCAAACGCGCACAGGCGGG | |
| | | GCAATCTTTTCGGGAAACGGCGGTGACTCGGTCTTTTGTTTCATGC | |
| | | ATAGCGCGACCCCGCTGGCCGATTTGATGTGTCGTCCGTCAGGTCT | |
| | | TACGCCGTTCATGCAAACATGGGCCGACGTGCAAAAGCTTACCCGT | |
| | | GCCTCAGCGACCGAAGTGCTGCGTCGCGCGTTAAAGACAGCCATGG | |
| | | CGCGTGGCTACATCTGGCCTGAATCCAATCTCCTCTTGTCCCGCGA | |
| | | CACAAGCTCGAGCCGTTTAACACCTGACTCCGTTCTGTCGAGCCTT | |
| | | GAGGGGATTCTGCCCGGTCGCTTGCGTCACCTCGCCCTGATTCGTC | |
| | | GTGCTCACAACACCTTCGAGCCATTCGCCCCTTGGCGTACGCCGCC | |
| | | AGTCGTTCACCCTCTCATGGCCAAGCCGATTCAAGCCTTCTGCCTT | |
| | | TCTCTTCCTTCATGGATGTGGGTCAGCGGTGGTAAAGACCGCTCGC | |
| | | TCGTGCGTGACGCGTTCGAAGGATTACTTCCAGATTCAGTGCGCCT | |
| | | TCGTAAATCAAAGGGAAGTCCTGCAGGCTTTCTGCATGCGCTGTAC | |
| | | CGCGCCAAGGGTCGTCAAATGATTGAGCGTATCCGTCACGGTTACC | |
| | | TGCGTCGTGAGGGGATCATCGATATCTCTACTGGCCCGGACGCATT | |
| | | GTTCTCGGAAGGGTTCCGCAATCCGCGTGTAATGCACCGTTTCTTT | |
| | | GAGCTCGCCGCAACTGAGGTGTGGATCGATCACTGGCGCAACTGGC | |
| | | GCCGCCCCCGCACA | |
| bamB | Codon | ATGGAAGGGTTGTATCAGCTGAAAGTGCATAGTCGTATACACAAAC | 468 |
| | optimized | TGCAAAATAATATCGCAATAGGTAGCATGCCGCCTCACGCGCTGAT | |
| | | CATCGAGGATGCCCCCGAATATTTGTCAAACGTTCTGCGCTTCTTT | |
| | | AGTAGCAAAAAGACTATAAAAGAAGCTGAAGTGTACCTGTCGGATA | |
| | | ATACGAATCTGAGCTCCAATGAGATCAACCTGTTGTTAGGTGATCT | |
| | | GATTGAGAACGAGATTATCGTAAAGCAAAACTACGACTCGAATAAT | |
| | | CGGTACAGTCGACACAGTCTGTATTACGAGATGATTGATGCCAACG | |
| | | CTGAAAACGCGCAGAAAATTCTGGCAGAGAAAACAGTGGGCCTCGT | |
| | | TGGGATGGGCGGGATTGGTTCCAATGTAGCCATGAATCTCGCAGCC | |
| | | GCCGGTGTTGGCAAACTGATCTTTAGTGATGGCGATACCATAGAAC | |
| | | TGTCTAATTTAACGCGACAGTATCTTTACAAAGAGGATCAGGTGGG | |
| | | CTTGAGCAAAGTAGAGAGCGCCAAAGAACAACTGCAATTACTGAAT | |
| | | AGCGAAGTCGAGCTTATCCCGGTTTGCGAAAGTATCTCTGGTGAGG | |
| | | AACTGTTCGACAACCATTTCTCCGAATGCGATTTCGTCGTACTGTC | |
| | | CGCCGACTCTCCGTTCTTTGTTCACGAATGGATTAACAATGCCGCG | |
| | | TTGAAATATGGCTTCTCCTACTCTAACGCAGGATATATCGAAACCT | |
| | | ATGGCGCGATCGGTCCACTGGTGATACCTGGGGAAACTGCCTGCTA | |
| | | CGAATGCTATAAAGACAAGGGCGATCTTTACTTGTACTCCGACAAC | |
| | | AAGGAAGAATTTTCTGTGAACCTGAATGAATCATTCCAAGCACCGA | |
| | | GCTATGGACCGCTTAATGCGATGGTTAGTTCCATTCAGGCGAATGA | |
| | | AGTGATACGCCACCTCCTCGGACTTAAAACCAAAACGTCCGGCAAA | |
| | | CGGCTGCTGATCAACAGTGAAATCTACAAAATCCACGAAGAGAACT | |
| | | TCGAGAAGAAGAACAACTGCCTGTGCTCGGATATTAAGGGCGAGAA | |
| | | GCTGTCGAAGAACACCCTTAACTCCGATAAAGAGCTGCACGAAGTG | |
| | | TATATCGAAGAACGCGAATCGGATTCTTTCAACTCCATTCTCTTGG | |
| | | ATAAAACCATGAGCAAGCTGGTAAAAATTAACAAAGAGGAGACAAA | |
| | | AATCCTCGACATTGGTTGCGCTACCGGCGAACAGGCTCTGTATTTC | |
| | | GCGAATAAAGGTGCTAAGGTGACCGCTGTCGACATTTCAGACGATA | |
| | | TGTTGAAGGTGCTGGACAAGAAAGCAAGCAACATTAACGCGGGGAG | |
| | | TATCAAAACCATGCGTGGTAATATCGAATCCATCGAGGTGAATGAC | |
| | | ACTTTTAATTACATCGTCTGTAACAACATCCTTGATTACCTGCCGG | |
| | | AGATCGACCGCACGCTGAGAAAACTTAACATGTTTTTGAAAAATGA | |
| | | CGGGACGCTGATTGTGACGATTCCCCACCCCGTGAAGGATGGTGGA | |
| | | GGGTGGCGGAAAGATTATTATAACGGCAAATGGAACTACGAAGAGT | |
| | | TTATCCTGAAGGATTACTTCAACGAGGGTCTGATCGAAAAGAGCCG | |
| | | CGAGGACAAAAATGGGGAAACGGTGATCAAAAGCATTAAAACGTAC | |
| | | CACAGAACCACCGAAACCTATTTCAATAGCTTTACTGACGCTGGCT | |
| | | TCAAGGTAGTATCTCTGCTGGAACCGCAACCGCTTTCAACTGTTTC | |
| | | AGAGACTCATCCAATTCTGTTCGAAAAGTGTTCGCGCATTCCGTAC | |
| | | TTTCAAGTTTTTGTGCTCAAGAAAGAGGATCGCCACGCCATT | |
| bsjM | Codon | ATGATCAAAAATGTAAACCTCAAAGAGGCCATTAAAGGTTTGACCG | 469 |
| | optimized | TATCAGAACGTTATGACACTCTGAAAAATTCGGGAGTCAACCTGAA | |
| | | TCTGAACATTTCGGCTTTGGAAGAGTGGCGCAACCGTAAGAATCTT | |
| | | TTAGCCGATGAGGACTTTACGGAGATGCTGACGGTGCTGGAATATG | |
| | | ACCCGGTGTATTTTAGCCACGCGATTAACGAGAACATCGAAGAACA | |
| | | TATCGATATCTACAAGAGCAAAATTCTGGGGGAAAACTGGTTTATC | |
| | | GTGCTGAACGATATTCTGGACGAGCTCGATAATCCCATCGAATACA | |
| | | AGAAAGAGATGAATCACAGCTACCTCCTGCGTCCGTTCTTGCTCTA | |
| | | CGCCGAAAAGGAGATGAACAAATACATTGTCAATCGTAAGGAGTTA | |
| | | CTTCCGGTGGAACCCCAGGTCATCCAACAGATCATGGAAAATTTGG | |
| | | CCTCCAAACTGTTCGCCGTTTCTGTGAAAAGCTTTGTCCTGGAGCT | |
| | | GAATATTTCGAAATTGAAGGACGAACTGGCCGGCGAAACACCGGAC | |
| | | GAACGCTTTCACTCATTTATTCGTTTGATGGGTGAGAAAACGCGCC | |
| | | TGGTGGACTTTTACAACGAATATATCGTTCTGAGTCGTATTCTGGT | |
| | | GAACATCACGATCTTATTCGTCAACAACATTATTGAGCTGTTTGAG | |
| | | CGCCTGCAGGAATCCAAGCTGGATATTGTTAAGAAACTTGGCGTGC | |
| | | AGGAGGAGTTCAAAATCAGTAATATTAGCATTGGCGAAGGTGATAC | |
| | | ACATCAGCAAGGACGCTCGGTTATCGTTCTTACGTTCGTGAGTGGA | |
| | | AAGAAAGTGGTGTATAAACCAAAAAATCTGAAAGTTGTTTCTGCTT | |
| | | ATAATTCTTTAATTGACTGGATCAACAATAAAAATAATATTCTGAA | |
| | | AATGCCTTCGTATAACACATTGATTTATGATGATTTCGTGATCGAG | |
| | | GAGTTTGTCGAGAAACGTGACTGCAAAAGTATCGAGGAGGTCAAAA | |
| | | AATATTATATTCGTTATGGGCAAATTTTGGGGATTATGTATATCTT | |
| | | AAATGGGAACGATTTTCATATGGAAAACCTGATTGCCTCGGGTGAA | |
| | | TATCCGATCATTGTTGACTTGGAAACGCTGCTTCAGAACATTATCA | |
| | | ATTTTAAAAACAAACCATCAGCGGACTTGATCACCACCAAAAAGAT | |
| | | GCTTAACCTGGTAAACAGTACTCTGCTGCTCCCTGAAAAACTTCTG | |
| | | AAGGGCGACATCACGGACGAAGGAATCGACATGTCAGCCTTGGCAG | |
| | | GGAAAGAACAACACTTGGAACGCCGCGAATACCAGTTGAAAAACCT | |
| | | GTTCACCGACAACATGGTTTTTGATCTCGAAAAAGTGAAAATCGAA | |
| | | GGTGCGAACAACATCCCGAAATTAAACGGTGAAAACGTTGACTACA | |
| | | GCACCTATATTGATGAGATTGTGGTTGGGTTCGAAAATATCTGTAA | |
| | | CCTGTTCATTCAATATCGCGACGAGTTACTGCATTCCGGCATCCTG | |
| | | GAGGAGTTTAAAGATGTGAAGGTTCGTCATGTGCTTCGCAATACGG | |
| | | TTGTTTATGCTAAGATGCTGGCGAATACATATCATCCAGATTACCT | |
| | | GCGTGATTCGTTGAATCGCGAACAGGTTCTTGAAAACATTTGGGTG | |
| | | CATCCGTTTGAGCGCAAAGAATTCATTAAGAGCGAGATGGAAGATA | |
| | | TCCTCAACAACGACATCCCGATCTTTTTCTCATACGCGTCGTCTAA | |
| | | GGATATTATCGATTCGAATGGCAAACTGCACAAAAACGTTATGGAA | |
| | | ATTTCGGGTTACGAACGTTTTACCACCAAACTGAAGGAACTGAATC | |
| | | CCTTTCTGATTGAACAGCAGGTGAGCGTTATTAATATTAAAACCGG | |
| | | CCGCTATGGGGATAAGAAATTCGAAAAAAATTATAGCGTGCGCGAC | |
| | | GTTGCAACGGAGAAAAAAGATAATCCGATTGATTTCCTGCAGGAGG | |
| | | CAATGAATATCGGCGATAAAATTTTGGAACATGCTATCATCTGTGA | |
| | | TGAGACCAAAACGATTTCGTGGCTTACCATTAACAACCATCATGAT | |
| | | AAAAATTGGGAAATTGGGCCTATTTCCGGTGAATTTTATGATGGTC | |
| | | TGGCGGGAATTTCACTCTTCTACCACTACCTCTATAAAAAATCCCA | |
| | | CAATGTCGAGTATAAAAAAATTCGTGATTACGCGTTCAACATGGCG | |
| | | AAAGTCAAAGCCCTGTCACTGAAATACGATAGTGGCTTGACCGGTT | |
| | | ACGCTTCCTTGCTGTATACGGCACACAAGATTGTTCAGGATGAACC | |
| | | GCGGAAGCAATACAAAGACGTGATCAACGAAGTGTTCAAGTACATT | |
| | | GATGAGAGCAAAGTCGTGACCGCTAAGTATAACTGGTTGCATGGCA | |
| | | CTGCCTCTATTATTCATGTGTTATTGAACCTCTACGAGGACTCTCG | |
| | | TGATATGGCGTACCTGACTAAATGTATTCAGTACGGCAAATATTTG | |
| | | GTCAAGCAAATCAAAGAACACAAGGATATGCTTGCGCCTGGCTTTA | |
| | | GCCAGGGCATCTCTTCGGTCATTATGGTTCTGGTGCGCTTAAGTAA | |
| | | AAAGTGTGAAGTCGAAGAATTTCTCGAATTAGCTCTGGAATTAATG | |
| | | GAAATGGAACGCAACAAACTGGGAAACCTTTCTGAATCAAACTGGC | |
| | | TGAACGGCTTGGTGGGCATTGGCTTATCACGTATCAAACTGAAAGG | |
| | | ACTGGATTCCAACTTACAGGTCGACAACGACATCGAACTCGTCCTG | |
| | | GATGGCGTCATGAACAGCTTGTACTCAAAAGATGATACTTTGAGCT | |
| | | GTGGTAACTCTGGCACAGTGGAATTGTTCCTGAGTCTGTTTGAACA | |
| | | GACGAAAAAGAAAGAGTATCTGGATATGGCGAAAGCAATCTGCGGG | |
| | | AAAATGATCGAAGAGAGTCGCATCTCCTTTGAGTATCAGACAAAGA | |
| | | GTCTGCCGGGTTTAGAACTGGTGGGCCTCTACTCTGGCTTAGCCGG | |
| | | AATTGGTTATCAATTCTTACGTATCTCGGACGTTGAGGATATTGCG | |
| | | AGCATTGCTACCTTAGAT | |
| capB | Codon | ATGCAGCCAGACCTGGAGGTTGTTGATGTTCGTCGCGGCGAGTCGT | 470 |
| | optimized | TCAAGGCATGGTCGCATGGGTACCCATATCGCACTGTTCGCTGGCA | |
| | | CTTCCATCCTGAGTTTGAAGTACATCTGATCGTGGAAACCACCGGC | |
| | | CAGATGTTTGTGGGTGATTATGTCGGAGGCTTTGGTCCGGGTAATC | |
| | | TGGTCCTGATGGGTCCCAATCTGCCTCATAATTGGGTGTCTGACGT | |
| | | TCCTGAGGGTAAAACCGTTGCAGAGCGTAACCTTGTTGTTCAATTT | |
| | | GGGCAAGCGTTCGTTTCCCGTTGCGAGGATTCCTTAACGGAGTGGC | |
| | | GTCACGTGGAAACGTTACTGGCGGATGCGCGGCGTGGCGTGCAATT | |
| | | TGGGCCGCGCACCTCTGAGGCCATTAAACCTCTGTTCGCGGAACTG | |
| | | ATTCACGCGCGCGGCCTGCGTCGCATTGTGCTGTTTCTGTCTATGC | |
| | | TGCAAATCCTCGTCGATGCAACGGATCGCGAACTGCTGGCATCTCC | |
| | | AGCTTATCAGGCGGATCCTTCGACATTTGCAAGCACGCGCATTAAT | |
| | | CATGCGCTGGCCTACATTGGAAAGAATCTGGCGAACGAGCTTCGTG | |
| | | AAACAGATTTAGCACGGCTGGCCGGACAGTCTGTTTCCGCCTTCTC | |
| | | TCATTATTTTCGTCGTCATACCGGCCTGCCTTTCGTGCAGTACGTT | |
| | | AATCGCATGCGTATCAACCTGGCCTGTCAGCTTCTGATGGACGGGG | |
| | | ACGCATCGGTGACAGATATTTGTTTCCGTAGCGGTTTTAACAACCT | |
| | | GTCCAATTTTAACCGTCAGTTTCTGGCAGTGAAAGGTATGTCACCC | |
| | | AGTCGGTTCCGTCGCTACCAGGCTCTCAACGACGCGTCACGTGATG | |
| | | CGAGTGAAGCGGCTGCAAAACGCGGCGCAGGTATTGCAGGTGCACC | |
| | | GGCAATCGTTCCAGCGGCTCAAGCACGTGGCGAGGCACGCCCAATT | |
| | | CCTGAAGTGCTGCTTAGCGGC | |
| capC | Codon | ATGATGCTGACGGCGAGCTCCACACCGGCATCCGGTAATCCAGCTG | 471 |
| | optimized | CCCGTGCATTGCGCGCCGCTGCCTTTGCACTGGCCTTAGGCGGAGC | |
| | | ATGCGTTGCGCATGCCGCACCTCTGCGGATTGGCATGACATTCCAA | |
| | | GAATTGAATAACCCGTATTTTGTGACCATGCAGAAAGCACTGAACG | |
| | | AAGCCGCGGCGAGCATTGGCGCGCAAGTGATTGTAACAGACGCACA | |
| | | TCACGACGTGTCAAAACAGGTATCAGACGTTGAGGATATGCTGCAG | |
| | | AAGAAAATTGATATTTTACTGGTGAATCCAACCGACTCCACGGGCA | |
| | | TCCAGAGTGCGATTGTTTCCGCAAAGAAGGCTGGCGCCGTGGTCGT | |
| | | GGCGGTCGATGCCAATGCCAATGGCCCGGTGGATTCCTTCGTAGGG | |
| | | TCCAAGAATTTTGATGCCGGCGCTATGTCATGCGAGTACCTTGCGA | |
| | | AAGCGATCAACGGCGGCGGCGAAGTGGCCATTCTGGATGGCATCCC | |
| | | GGTCGTCCCAATCCTGGAACGTGTCCGCGGCTGCCGCGCGGCACTG | |
| | | GCCAAATTCCCGAATGTGAAAATTGTCGACGTTCAGAATGGAAAAC | |
| | | AGGAACGTGCGACAGCGTTAACGGTAACCGAGAATATGATCCAGGC | |
| | | GCACCCGAAACTGAAAGGTGTGTTTAGTGTAAACGACGGCGGGTCA | |
| | | ATGGGCGCTTTGAGCGCCATTGAAGCGAGCGGCAAAGATATCCGCC | |
| | | TCACGTCCGTAGATGGTGCCCCAGAGGCGGTGGCGGCGATTCAAAA | |
| | | GCCGAACTCCAAATTTATTGAAACAAGCGCTCAATTTCCGCGCGAC | |
| | | CAGATCCGTTTAGCGATTGGTATTGGCCTGGCCAAGAAATGGGGCG | |
| | | CGAACGTGCCAAAAGCGATTCCAGTCGACGTGAAACTGATTGACAA | |
| | | AGGGAACGCGAAAACCTTTAGTTGG | |
| cinX | Codon | ATGGCTCTCAAAACCTGCGAAGAATTTCTGCGCGATGCGTTAGATC | 472 |
| | optimized | CGGATCGCTTCGGCCGCGAGATGAAGGCAGTAACAGAAATTCCCGA | |
| | | GATCGTTAAACTCGGCCATCGTCATGGTTATGGATTTACTGCCGAA | |
| | | GAATTTCTGACCAAAGCTATGAGTTTTGGTGCTCCGCCGGCAGGAG | |
| | | CAGCAGCACCTGGCGAATCAGCCAGCGTTCCTGGCCAGAACGGTTC | |
| | | CTCCCCCGGACACGCTGCGCGTGCAGCTATGGCTGGTCCAGAAGCA | |
| | | GGGGCCACCAGCTTTGCCCACTATGAATACCGTCTGGATGAGCTGC | |
| | | CGGAATTCGCCCCCGTTGTGGCCGAGCTTCCGAAACTGAAAGTCAT | |
| | | GCCGCCTTCCGTGGGACCTGATCGGTTTGCAGCACGCTACCGTGAT | |
| | | GAAGATATGCGCACAATTTCAATGAGTCCGGCGGATCCGGCTTACC | |
| | | AGGCTTGGCACCAGGAACTGGCGGGTCGTGGTTGGCGCGATGCAGA | |
| | | AGATACGGCTGCTGCTCCAGATGCCCCACGGCGCGATTTTCATCTG | |
| | | CTGAACCTCGATGAGCATGTAGATTACCCAGGTTATGAAGAATATT | |
| | | TTGCGGCCAAGACCCGTGTCGTCGCGGCACTCGAAAACCTGTTTGG | |
| | | TGGTGACGTGCGTTGCTCAGGCTCTATGTGGTATCCGCCGTCGAGC | |
| | | TATCGCTTATGGCATACAAATGCCGATCAACCGGGGTGGCGTATGT | |
| | | ACCTGGTAGATGTAGATCGCCCATTCGCGGACCCCGACCGTACCTC | |
| | | CTTCTTTCGCTACCTGCATCCACGTACCCGTGAAATCGTCACGCTG | |
| | | CGCGAAAGCCCTCGTATTGTCCGTTTCTTTAAAGTCGAACAGGATC | |
| | | CCGAGAAGCTGTTCTGGCACTGTATCGCGAACCCCACCGATCGCCA | |
| | | TCGCTGGTCGTTTGGTTACGTTGTTCCGGAAAACTGGATGGACGCC | |
| | | CTCCGTCACCATGGC | |
| cln1B | Codon | ATGCCTTTATGGTTAGCGCAGGACGTCCACGCGGTCGCTCTGGACG | 473 |
| | optimized | AAGATATCGTGGTGCTGGATGCGGTGAGCGACGCATACCTGTGTTT | |
| | | AGTTGGTGCCAGCGCTCTGATCAGCTTGGGCAGCGAGCGTTCCGTC | |
| | | AGTGCAGATCCGGTGGCCGCTGAGACACTTCGTGAGGCTGGTCTGG | |
| | | TGGGTCCACATCCTAGCGGCGCCACCCGACCAATACCTCCGAAGCC | |
| | | GACGATTGACTTACCTGATGCAGCCCGTCAGGCGCAAGGTCGTGAA | |
| | | TTACGTGCCGCCGCGTGGGCTGGCGCGGCAACCGCAATCGATTTCC | |
| | | GCCGGCGTTCATTTAGACAACTCCTCGCGAGAGCAGGGCAACGCCC | |
| | | GCCGGGTCAAGCAGCTGCTCCGGCTGATGAGGTATTGGCAGCAGCC | |
| | | GCAGTGTTCATGCGGTTACGTCCATGGTCACCCGTTGGAGGCGCGT | |
| | | GCCTTATGCGTTCGTATTACTTATTACGGCATTTGCGCATCCTCGG | |
| | | TTTCGATGCCGATTGGATCATTGGTGTGCGTACGTGGCCATTTATG | |
| | | GCCCATTGCTGGCTGCAGGTCGGTGCCGTCGCACTCGACGATGACG | |
| | | TCGAGAGATTAACAGCATACACACCGATTCTGGCGGTG | |
| cln1C | Codon | ATGGGCGACTACCTGGCTCTGTACTGGCCGCGCGGCATGCCCGGTG | 474 |
| | optimized | TAGCTGCAGACGCAATGCGGGCCGCCATCGAAGCTGAGGGCGCCTG | |
| | | GACCCTGGCGTTCGAGGCCTACCAGCTGGTAGTGTATGTCAAAGGG | |
| | | CCCCGAGCACCTAAAGTGCGTGCCCTGCCGGATCAGGGCGGGGTGG | |
| | | TCATTGGGGAACTGTTTGATACTGCAGCAACCCGCGAAGGACGCGT | |
| | | GCAGGACTTTCCTATAGCGCTGATCAAAGACGTCGCAGCTCAGGAT | |
| | | GCCGCACGTATTCTTGCTACCCATGCGTGGGGTCGTTATGTGGCTG | |
| | | TATTAAAAGCCGGTGATCGTCCGCCATGGATCTTTCGCGATCCAAG | |
| | | CGGGGCGGTGGAATGTCTGGCGTGGGTCCGCGATGAAGTGACCATC | |
| | | ATTAGCAGCGATGTTGCAGCGCAACGAGCTTGGTCCCCTGATCGGC | |
| | | TGGCGATTGACTGGTCGGGACTGGGACGTGTACTGGCACGCGGAAA | |
| | | CTTATGGGGAGAAATTTGCCCGCTGGCTGGCGTCACGGCGATTGCG | |
| | | CCAGGTACCGCACGGTGTGATCTCGGTGATGCAGCTCTGAGCCTGT | |
| | | GGCGCCCAGGAGATCATGCACGTCGTAGTCGTCATGATGTTTCCCC | |
| | | ACGTGATTTGGCAAGAGTGGTGGATGCTAGCGTTGCAGCCCTGGCT | |
| | | AGAGATCGCAGCGCTATTCTGGTCGAAATCAGCGGGGGACTGGATT | |
| | | CCGCTATCGTTGCCACGTCGCTGGCTCGTTGTGGAGCCCCAGTTGT | |
| | | TGCTGGAATTAACCATTACTGGCCCGAACCGGAGGGTGATGAACGT | |
| | | CGCTGGGCCCAGGACATCGCAGATCGGTGCGGTTTTCGCCTGATCG | |
| | | CGGGCCAACGTCAGCGGCTGTTGCTGGACGAGGCAAAGCTGCTGAG | |
| | | ACATGCACAGGGCCCGCGACCTGGTCTGAATGCGCAGGACCCGGAC | |
| | | CTCGATCACGATCTGGCGGAACAGGCTAAAGCGTTGGGTGCCGATG | |
| | | CACTGTTCTCAGGGCAAGGTGGCGATGGTGTGTTCTATCAAATGGC | |
| | | AAATGCTGCACTGGCAGCCGATATCCTCATGGGGAAACCTGCTCCT | |
| | | ATGGGTAGAGCCGCGTCTTTAGCCGCTGTGGCTCGTCGGGCACGAG | |
| | | CCACGGTCTGGAGTTTGTGCGGCCAGGCTATGTTTCCGTCGCGCGC | |
| | | ATTTGCCGCTGGTATGCCGCCGCCAAGTTTCTTGAGCGCCGGTTTG | |
| | | GCGCCGCCACCCGTGCACCCGTGGATTGCAGACCAGCGCGGTGTTT | |
| | | CACCGGCGAAACGTATTCAAATTCGGGGGCTGACCAATATTCAATG | |
| | | TGCTTTCGGCGATAGCTTACGGGGCCGAGCAGCAGATCTTTTATAT | |
| | | CCGCTTATGGCCCAACCGGTCATGGAACTGTGTCTGTCTATCCCTG | |
| | | CACCGCTGTTGGCAGTAGGCGCATTGGATCGCCCTTTCGCACGTGC | |
| | | GGCGTTCGCAGATCGATTACCTCCTCGTTCACTCGTTCGACGCTCA | |
| | | AAAGGTGATGTTACCGTGTTTTTCAGCAAAAGCCTTGCAGCAAGCC | |
| | | TGCCGGCCCTTCGTCCTTTCCTGCTGGACGGGCGCCTTGCAGAACA | |
| | | GGGTCTGATCGATCGAGCAAAACTGGAACCTCTGCTGCACCCCGAA | |
| | | CCGATGATTTGGCGCGACTCAGTCGGCGAGGTAATGCTGGCAGCGT | |
| | | ATCTTGAAGCCTGGGTGCGCGCATGGGAAGCCAAGTTGCGTGTTAG | |
| | | C | |
| cln2B | Codon | ATGACTCTGACCTGGCGCCCGGGTGTTCACGCGGTAATGGTCGAAG | 475 |
| | optimized | ATGATCTGGTTCTGCTGGATGAAGCAGCGGACGCTTATGTCTGTTT | |
| | | GTTGGATGGCGCCAAAGTGGTTAGCGTCCGGGCTGACGGTGCTCTG | |
| | | AGCTTCAATCCCCCACATGCAGCAGAAGATATGATCGCGGGTGGCC | |
| | | TCGTCGAACCTTCATCAAGTGCCGCGGCGTCAGCAAACCCGCCGGC | |
| | | AAAACTCCCATGTACTCCGCTGGCGCGCTTATCGCGCCCGCGGCAT | |
| | | GTAAAAGTGCGTCCGGCTGAAGCGGCCTTGTTCCTGATCCAAGCCT | |
| | | GGGGTGTTGCGCGTGCGGTACGTCGTTGGCCAATGGCTAGATTATT | |
| | | AGAAGCATTACGTGGAGATCGTGCCGCAGAACCGGCGAAAGGCCGC | |
| | | CGATCGATGGCGGAGGCGTGCGCTGTTTTTGATGCGCTTCTGGCCT | |
| | | GGAGCCCTTTTGACGGTGAATGTTTGTTTCGCTCAGTATTACGACG | |
| | | TAGATTTTTAATGGCACTGGGCCATTCGCCGGACTTGGTGATAGGC | |
| | | GTGCGTACCTGGCCGTTCCGCGCACATTGCTGGCTGCAGAGCGGAG | |
| | | TGGATGCCCTGGATGATTGGCCGGAACGGCTCTGCGCATATCGCCC | |
| | | GATTCTGGCAGCTTCTGCAAGCCAGGGTAGA | |
| cln2C | Codon | ATGAGTTACCTGCTGATGACCTGGCCGCCGGGGCAGCCGAGCGTAG | 476 |
| | optimized | AAGCTGATGCACTTCACGCAGCCTTTAACGGGCAGGGTGGATGGAG | |
| | | CCTGGTTTTGGAACGATTCTGCCTGCGCGTATACGTGCGTGGCGCG | |
| | | GCAGCCCCTGCAGTTACCCTTACCCCGAAAGGAGGCGTGCTCATTG | |
| | | GTGAGATGTTTGATCGGGCTGCCACAGAAACGGGCGCCGTTGCCGC | |
| | | TTATGATCTGAGCCGCCTGGGAGATGACGACGGTATGGCCGTAGCC | |
| | | CGGCGTGTGGTGGACGAAGCGTGGGGGAGATATGTGTTGGTGCTGC | |
| | | CAGTTAAAGAACGCCGTCCAGTGGTTTTGCGAGAACCACTGGGCGC | |
| | | GCTGGATGCGCTGATCTGGCGCAAAGGCGATGTCTGGTGCGTGGGG | |
| | | GCAGACGTACCCCCGGGTCTTGAACCAAAAGATCTGGGTGTGGAAG | |
| | | AGACTAGACTGACGCACCTGATCGCGGAACCGGATCTGGCATCTGC | |
| | | GAGCCTGCCCTTAACCGGCGTCGCGGCAGTGATGCCAGGTACTGCG | |
| | | GTCGATGAAACCGGCCAGGTGCACCGTCTGTGGACCCCCGCGCGTT | |
| | | TTGCTCGCTCCCCTCGCACTGACGCGTGGACTGCAGCCGAACGTAT | |
| | | TCCGCTGGTTACCCGTGCGTGCATCGCGGCGCTGTCTGCGAATCGA | |
| | | AGTGGTATTCTGTGCGAGATTTCGGGCGGCCTGGATAGCGCTATTG | |
| | | TTGCGACCTCTCTGAAAGCGGAAGGTGCGAAGATTAGTAGCGGGAT | |
| | | CAACTTCCATTGGCCCCAGGCTGAAGCAGATGAGCGCCCGTACGCA | |
| | | CGCGCTGTTGCGAAAAGCGTGCGAACCCGGTTACAGGTGGTAGCGA | |
| | | GTCGTGTAGCGCCCGTTGACCCGGAAACGTTTGATGAGATCGTGGT | |
| | | CGCGCGACCAAGTTTTAATGCCATTGATCCAGTCTATGATACCGTA | |
| | | CTGGCCCAACGTCTGATTCAGGGCGGTGAAGGAGCCCTGTTTACCG | |
| | | GACAAGGTGGTGACGCAGTTTTCTATCAGATGCCAGCACCACAACT | |
| | | TTCGTTGGATTTGTTGGCTCGTGGCCCCCGCCGCCGCGGTCTTATG | |
| | | GGATTATCACGCCGCACCAACCGCAGTGTCTGGTCGTTGCTGCGCA | |
| | | TGGGCTTACGTGCACCCGTACGAGCAACCTTTCCCTACGGTGCGAG | |
| | | AGGTGCCGATCGTCCTCCGATGCACCCGTGGCTGGAGGACGCGCGT | |
| | | GGTGTTGGGGCCGCGAAACGGATTCAGATCGAAGCGCTGGTTGCTA | |
| | | ACCAGGCCGTGTTTGAAGCATCTCGTCGCGGTGCGGCGGCTCATTT | |
| | | GGTGCACCCACTGCTGTCGCAACCGCTTGTGGAGCTGTGCCTTTCA | |
| | | ACCCCAGCGGCCGTGCTGGCGGGTGCCGAACAAGATAGAGCATTCG | |
| | | TGCGTAGCGCTTTTCGTGCGCAACTGCCACGCCTGGTCTTAGATCG | |
| | | TCAAAGCAAAGGAGATCTGAGCGTTTTCTTTGCTAAAGGTGTGGCG | |
| | | CGGAGCTTGCCGGGCTTGCGTCCGCGTCTGCTCGAAGGACGCTTAG | |
| | | CGGCACGTGGCCTGATCGACGTGGAAGCGTTATCACAAGCGATGCA | |
| | | GCCAGAAGCGATGATTTGGCGTGACGGTTCGGCCGAAATCCTGTGC | |
| | | CTTGCTGTTCTGGAATCATGGCTCCGCTCTTGGGAGGCTCGTGGTG | |
| | | CA | |
| cln3B | Codon | ATGCGCGTTGCAGTGCCGGATCATTTAGCGTATTGCGTAAAACAAG | 477 |
| | optimized | GTGGAGTTACGTTTCTGGACGTCCGCGGGGATCGTTACTTCGGCCT | |
| | | GCCGCCGGTGCTGGAACACGCGTTCGTTGCCATTGCCGAGGCGGAT | |
| | | TTTCTGCTGAAAGAACCAAATTCACTTCTGGAGCCACTCGAAGCAC | |
| | | TGGGTGTCTTAGTGCGAGGCCAAGCCCGCCGTGCCGATCTGACAAT | |
| | | TCCGTCTGCAAATCTGTCATGGGTGGATGAGGTCAGCCCGACCCCA | |
| | | CCACGTCTTGACCCTGCGTCACTCGTCGCAACCGTCACGTCTGTTA | |
| | | TTCGAACGCGTCTGAGCCAAAAGAGTAAGTCCTTGCAGGCTCTCTT | |
| | | GGAAGAGGTCCGTACCCGCCGTCCGGGATCGCCGGCCCATAATTGG | |
| | | CAGCTGATGCGTCGTCTGACGGCTGGATTCCGTGCATCGCGTGCTT | |
| | | GGGCGCCGATAGAACCCATCTGCCTCCTGGACAGCTTGGCGTTACT | |
| | | GGATTTTCTGCATCGCCGTGGCCTGTATCCGCATATTGTTTTCGGT | |
| | | GTGATCCGCCAACCGTTTGCCGCTCATTGTTGGGTGCAAGCTGATG | |
| | | ATGTAGTCCTGAATGACCGGCTGGATCATGTCGGTGAATATACACC | |
| | | GATCCTGGTGGTC | |
| cln3C | Codon | ATGGAAGATTACGTGGTCCTCATTTGGCCGGCACTCGCTGAAGCTC | 478 |
| | optimized | CTGCACGCGACTTGATTCGTCGCCTGCCGAAACTCAAAACCGTCAT | |
| | | TGAAACTAGCGGATTGGTGGTACTGCGCCCCGAAAATGGTGCGGGT | |
| | | CTGCGGGTAGGCGGGAACGGTGTGGTCCTGGGTAGCGTCTTTCGCA | |
| | | CCGGCGGTGATCGCGAAACTGTTGCGGAATTTTCGGAATCGGAAGC | |
| | | ATCCGCGATCGCCACGAGTCGTGGTCAGCAGTTAGTGACAGAGTTC | |
| | | TGGGGTGGCTACCTGGCTGTTCTTGGAGATGCTTCGCGTTCCGAAG | |
| | | TGATGGTCCTGCGAGATCCTTCAGGTGCAATGCCGGCTTATTGTTT | |
| | | AGTTCATGGCGAAGTCCAGATCATCTGCTCTCGCTTGGAGGTCCTG | |
| | | GAGGACGCAGGACTGGGGCAGCAGGCGCTGAACTGGGACGTGGTGG | |
| | | CGCAATTACTGGCCTTCCCAAACCTTCGAGGTCGCTCAACGGGTCT | |
| | | TAAAGGCGTGGAAGAATTACTTCCCGGTTGCCGTCTGACATTTACG | |
| | | GGAGGACTGAAAACCGAAACGCTGACCTGGAACCCGTGGCTTTTTG | |
| | | CCCGCCCATCTGCGCAAGCGCCTGAACGTGGAGTTGCGGCGACCGC | |
| | | CGTGCGTCAGGCGGTGGAAGTAAGCGTTCGAAAATGGGCTGATCAG | |
| | | AGTTCACCGGTACTTTTGGAATTGTCAGGCGGGCTGGATAGTAGTA | |
| | | TCATCGCCTGCTGTCTGGACGAACCGCGCACCGCGGCCACCTTCGT | |
| | | GAACTTTGTCACACCGACGGCCGAAGGCGATGAACGAGGATATGCA | |
| | | CGTCTGGTTGCCAAGGCAGCAGATAAACAACTGATCGAGCAGGACA | |
| | | TCCGGGCTGACGAAGTAGATGTTACCCGTCCAAGACCTGGCCGCCA | |
| | | TCCTCGTCCGGCCAGTCAGGCGCTGTTACAGCCGCTGGAACAGGCT | |
| | | TGCGCTGAACTGGCACCTCAGTTGGGTGCGAGAAGTTTCTTCTCCG | |
| | | GTCTGGGAGGAGACAACGTGTTTTGTAGCATTGCAACCGCAAGCCC | |
| | | GGCTGCGGATGCACTTTTGACTAGCGGTCTGGGCCGACAGTTCTGG | |
| | | GCCGCAATCGGGGACCTGTGTGCACGTCATAACTGCACCGTATGGG | |
| | | CAGCCTTAAGCGCCACGCTGAAGAAACTGCTCCGCTCAGATCGTCG | |
| | | TCTGGTGATCAAACCAAACCTGGATTTTCTGTCCTTTCGGGAGGAC | |
| | | GCCATAGACCGTCCGGATCACCCATGGCTTGAAGTGGCCGCCGATC | |
| | | GTCTGCCGGGGAAACGCGAACATGTCGCAAGCATTCTGTTGGCGCA | |
| | | AGGCTTCCTGGATCGTTATGAGCACGCTCAGGTTGCTGCCGTCCGC | |
| | | TTTCCCTTGTTAACGCAACCGGTTATGGAGGCTTGTCTGCGCGTGC | |
| | | CGACCTGGATGGCAAACCACCAGGGTCGCAATCGGGCGGTCGCACG | |
| | | CGATGCCTTCTTTGATCGCTTGCCCCCGAGAGTACGTGATCGGCAG | |
| | | ACAAAAGGAGGTTTGAACGCGTTTATGGGTGTTGCGTTCGAACGCA | |
| | | ACCGTCAGGCCTTAGCTCGTCATCTGTTAGACGGGCGCCTGGTACA | |
| | | GCGTGGCCTGATAGATGCAGTGGCAATAAAATCGGCGCTGGCCTCA | |
| | | CCAGTCCTGGAAGGAGGAGCCATGAACCGCTTACTGTACCTGGCCG | |
| | | ATGTCGAATCCTGGGTACGCTCATGGGAAGATGTG | |
| comQ | Codon | ATGAAGGAAATCGTGAAACAGAATATCAGTAACAAAGACCTGTCGC | 479 |
| | optimized | AACTCCTGTGTTCCTTCATTGATTCAAAGGAAACTTTCAGTTTTGC | |
| | | CGAGAGCGCTATACTGCATTATGTAGTATTCGGCGGTGAGAACCTG | |
| | | GACGTAGCTACCTGGCTGGGCGCCGGAATTGAAATTCTGATCCTGA | |
| | | GCAGCGATATCATGGACGACCTGGAGGACGAGGATAACCATCATGC | |
| | | GTTGTGGATGAAAATTAACCGCAGCGAGAGCTTGAATGCGGCCCTG | |
| | | TCCTTATACACCGTCGGCTTAACGAGCATCTATTCCCTGAACACAA | |
| | | ATCCGTTGATATTTAAGTATGTGCTGCGCTACGTCAATGAGGCCAT | |
| | | GCAGGGTCAGCATGATGATATAACCAATAAAAGCAAAACCGAAGAT | |
| | | GAATCGCTTGAAGTGATTCGCCTTAAATGCGGCAGCCTGATCGCCC | |
| | | TGGCAAATGTCGCGGGCGTGCTGTTAGCCACGGGCGAGTACAATGA | |
| | | AACAGTTGAACGTTACTCTTATTACAAAGGCATCGTGGCGCAAATT | |
| | | TCCGGCGACTATCACGTGCTGCTGTCAGGAAACCGGAGCGATATCG | |
| | | AGAAAAACAAACAGACACTGATTTACCTGTATCTGAAACGCCTGTT | |
| | | TAACAACGCGAGCGAGGAATTGCTGTATCTGTTCTCCCATAAAGAT | |
| | | TTGTACTATAAAGCCCTGCTCGACCGTGAAAAGTTTGAAGAAAAAC | |
| | | TGATCCAGGCCGGGGTGACGCAGTACATCAGCGTTCTGCTCGAAAT | |
| | | ATATAAGCAGAAGTGCTTCTCCACCATAGAACAGCTGAACTTAGAT | |
| | | AAAGAAAAGAAAGAGCTGATCAAGGAGAGCCTGCTGTCATATAAGA | |
| | | AAGGCGACACCCGTTGCAAGACC | |
| crnM | Codon | ATGAATGATATCAACAAAAACAAAACTAAAACCATTAACGAAAAGA | 480 |
| | optimized | TTAAAATTTTCACCAAAGAAGAGGTGATTGATATCAGTTACTTTGA | |
| | | AGAATGGCGCAGCGTTCGTACTCTGCTTAACGAAAACTACTTTAAA | |
| | | ATTATGCTCGAGGAAATGAATATTTCCAAAAACCAATTTTCGTATG | |
| | | CGCTGCAACCGTTAAACGACGAGTTCAAACTGCATACTAACGTTAA | |
| | | AAATGAAGAATGGATCAAATGCTTTAATCGCGTCATTAACAATTTT | |
| | | AACTATAAAAATATTAACTATAAAGTTGGTTTGTACCTGCCTATTC | |
| | | AGCCTTTCTCCGTTTATTTACAGGAGAAACTGAAAGAGATCCTGAA | |
| | | GAAGCTGAACAACATTAAGATTAATGATAAAATTATCGACGCCTTT | |
| | | ATCGAAGCTCACCTGATCGAAATGTTCGACCTCGTCGGTAAAGTAA | |
| | | TCGCCCTTAAATTTGAAGATTATAAACAGATCAACTTCCTGAAAAA | |
| | | CACAAATAATGGCACCCGCTTGGAGGAATTCTTGCGTAGCACCTTT | |
| | | TATTCTCGGAAGTCATTTCTGAAACTGTTTAACGAGTTTCCGGTAC | |
| | | TCGCGCGGGTTTGCACCGTACGTACGATCTATTTGATCAATAACTT | |
| | | TAGTGCTATCATCCAGAACATCAATAGCGACTACCTGGAAATCCAG | |
| | | GAATTTCTGAACGTCGATTTCCTGAACTTGACAAACATCACTCTTT | |
| | | CGACGGGTGATTCCCACGAACAGGGTAAAAGTGTGTCCATCCTCTA | |
| | | TTTTGATGAAAAAAAGCTGATTTATAAACCGAAAAATCTGAAGATT | |
| | | TCAGAAATTTTCGAGAGCTTCATCGACTGGTACACCAACGTCTCTA | |
| | | ACCATAAGCTGCTCGACCTGAAAATCCCGAAAGGAATTTTTAAAGA | |
| | | CGATTACACTTATAACGAATTTATTGAGCCAAACTACTGCGAGAAT | |
| | | AAGCGCGAAATTGAAAATTACTATAACCGTTATGGGTACCTGATCG | |
| | | CAATCTGTTATCTGTTCAACCTGAATGACCTGCATGTAGAAAATGT | |
| | | GATCGCCCATGGCGAGTACCCGGTTATTGTTGATATTGAAACGAGC | |
| | | TTTCAAGTCCCTGTGCAAATGGAGGACGATACTTTATATGTGAAGC | |
| | | TGTTGCGCGAGCTGGAATTGGAAAGCGTTTCATCGTCGTTTCTGTT | |
| | | ACCTACCAATCTGTCGTTTGGTATGGACGATAAAGTGGACCTGTCC | |
| | | GCGCTGAGCGGAACCATGGTCGAGCTGAATCAGCAAATTCTGGCGC | |
| | | CTGTCAACATTAATATGGACAACTTTCATTACGAGAAATCACCGAG | |
| | | CTATTTTCCAGGCGGAAACAATATCCCTAAAAACAACAAATCAGTG | |
| | | ACTGTTGATTATAAAAAATACTTGCTCAATATTGTGACTGGTTTCG | |
| | | ACGAATTTATGAAGTATACCCAAGAAAATCAGCTGGAATTTATTGA | |
| | | GTTCCTGAAAAAATTCTCAGATAAAAAAATCCGGGTGCTGGTGAAG | |
| | | GGTACGGAAAAATATGCGTCCATGATTCGCTACAGCAACCATCCGA | |
| | | ACTACAACAAAGAAATGAAATATCGCGAGCGTCTCATGATGAACTT | |
| | | GTGGGCGTACCCTTACAAAGACAAGCGTATTGTTAATAGCGAAGTA | |
| | | CAGGACCTGTTATTTAACGATATCCCGATCTTTTACTCCTTTCCAA | |
| | | ATAGCCGTGACCTCATTGATAGTCGCGGCTTGGTGTATAAAGATTA | |
| | | CCTTCCTGTGACAGGACTGCAGAAAGCAATTGATCGCGTGAAAGAT | |
| | | ACCTCGGTAAAAAGCTTGTTCGACCAGAAGCTGATTCTTCAGAGTA | |
| | | GCTTAGGTCTGTGGGATGAGATTCTCAACAAGCCGGTCCAGAAAAA | |
| | | GGAACTGCTCTTTGAAAAGCAGAACTTTAACTATGTGAAAGAGGCG | |
| | | ATCAATATTGCGGAATTGCTGATTGGCTATTTAATCGAAACGGACG | |
| | | ACCAGAGCACCATGCTGAGCATTGATTGTTCTGAAGATAAACACTG | |
| | | GAAGATTGTTCCTTTAGACGAATCCCTGTATGGTGGGCTGTCCGGC | |
| | | ATTGCATTATTTTTTCTCGATATTTATAAAATTACCAAAGATGAAA | |
| | | AATATTTTAATTACTATGATAAAATCATTTCCACGGCCATTAAACA | |
| | | ATGTAAAGCGACCATCTTCTCGTCAAGCTTCACGGGTTGGCTGAGT | |
| | | CCCATTTATCCGTTGATTCTGGAAAAGAAATACTTTGGTACCATGA | |
| | | AAGATAAGAAATTCTTTGACTACACGATGGAAAAGCTGTCGAATAT | |
| | | GACTGAAGAACAAATTAACAACATGGATGGTATGGACTATATCAGT | |
| | | GGCAAGGCGGGTATTGTCAAACTGCTGATTAGCGCGTACCGGGAAT | |
| | | CGAAGAACAATGAAAACATCGGACTGGCCCTGAGTAAATTCAGCAA | |
| | | CGATCTGATTCAAAATATTGGCACCGGCAAAGTCAGTGAATTACAA | |
| | | AACGTGGGCCTGGCGCACGGCATTTCTGGTATTATGGTCGTAGTAG | |
| | | CCTCACTGGACACGTTTAAAAGTGAATATATTCGCGAGCAGCTGGC | |
| | | AATTGAATATGAGATGTTCTGTTTGCGTGAAGATTCATACAAATGG | |
| | | TGTTGGGGCATCTCTGGAATGATTCAAGCCCGTCTCGAAATTCTGA | |
| | | AACTGAGCCCGGAGTGTGTGGATAAAAAAGAGCTGAACTTGCTTAT | |
| | | TAAGCGTTTTAAAAACATCTTGAATCAGATGATTAACGAAGATTCC | |
| | | CTTTGTCACGGCAACGGTTCGATCATTACTACGATGAAGATGATCT | |
| | | ATATGTACACCCAAGACACCGAGTGGAACTCTCTGATTAATCTGTG | |
| | | GTTATCAAATGTAAGTATCTATTCGACCTTACAAGGCTATAGCATT | |
| | | CCAAAGCTGGGCGATGTAACAATTAAGGGGTTGTTTGATGGCATTT | |
| | | GTGGTATTGGCTGGTTATACCTGTATTCGAACTTTAGCATTGAAAA | |
| | | CGTGCTGCTCCTCGAGGTC | |
| csegB | Codon | ATGGACCTGTGGTTGAGCGCCGGGGTCTATGCTGTCATGATCGATG | 481 |
| | optimized | ATGATGTAGTTTTCCTGGACGTCGCCACCAATGCATACTTCTGCCT | |
| | | CCCAGCCGTTGGGAGCGTGTTGGCACTCGAAGGTCGTTCGCTGCGT | |
| | | GTGGCGGCTCGCGAACTGGCAGAAGATCTTATTCAGGCAGGCTTAG | |
| | | CATCCGCGGCTGCGGCAATCGAACCCCCACCGAGCACACCAGCCCC | |
| | | AGTTCGCACTGCGCGTGCGGTATTGGAAGCTCTGCCGGCGCGTGAA | |
| | | AGACCACGTCCACGTCTTGCCCACTGGCGTCAGGCGATTATGGCTG | |
| | | GCTTGGCGTCCCGTGCCGCTGAACGTCGACCATTCGCGCAGAGACT | |
| | | GCCGCCGCCTTCAACGGGGGTTTCACCTCCGGCATCAGAAGGCCTG | |
| | | CTTGCCGATCTGGATGCGTTCCGTCGACTTCAGCCATGGTTGCCGT | |
| | | TCGACGGTGCTTGTCTGTTCCGTAGCCAAATGCTGCGCGATTATCT | |
| | | CCTTGCGCTGGGTCACCGCGTTGACTGGATTTTCGGTGTACGTACG | |
| | | TGGCCGTTTGGTGCCCACTGTTGGTTGCAGGCCGGCGACCTGGTGC | |
| | | TGGATGATGAGGCCGAACGTCTGATTGCGTATCACCCCATTATGGT | |
| | | AGGT | |
| csegC | Codon | ATGGGGTATGCCGCATTGACTTATCCGGGTGGTTTAGCGGCAGCAG | 482 |
| | optimized | CGTTTGATGAGATGGTAGAAGCACTGATCGATGCTGGATGGACCTT | |
| | | GGCGTTGCGTGCGTTCAGACTCGCCGTTCTCACCGATGGTCAGGCT | |
| | | CCAGCCGTGTCGCCGCTGATGGGCAGAGGCGGCGTAGCAGGCGTTC | |
| | | TCATCGGCGAAGCGTTTGATCGTCGCGCCACATTAGGTGGCGCGGT | |
| | | CGCACGTGCCGCGCTGGATGGTTTGGCTGACATCGATCCGCTGGAA | |
| | | GCAGGTCGCCATCTGATTGAAACCGCGTGGGGCGGCTACGTGGGTA | |
| | | TGTGGATTGGTCGGGCCGAAGCTGGTCCGACACTGCTGCGCGATCC | |
| | | TAGTGGCGCGCTCGAAGCCTTAGCGTGGCGCCGTGACGGTGTAACC | |
| | | GTTATGTCAGCGCGCCCGTTGACGGGGCGCGCAGGCCCAGCTGATT | |
| | | TAGCAATCGATTGGCCACGTATCGTGCAGATTCTGGCCGATCCCAT | |
| | | TTCCGCGGCTCTCGGCCCGCCCCCTCTGACTGGCTTAGCGACCATA | |
| | | GACCCGGGCGCGGCGGTTCATGGCGCGGATGGCCAAGAACGCTCAG | |
| | | TGCTGTGGACCCCAGCTGCAGTTGTCCGTGGTGCTCGTCACCGTCC | |
| | | TTGGCCAAGCCGTCAGGATCTGCGTCGCACCATCGATGCGACTGTC | |
| | | GCGGCACTGGCCTCGGATGCGGGCCCGATTGTCTGCGAAATTTCAG | |
| | | GAGGTCTGGACTCGGCCATAGTTGCGACTAGCCTTGCGGCGTCCGG | |
| | | TCTGGGTCCGCAGCTGACAGTGAATTTTTACGGTGACCAGCCTGAA | |
| | | GCTGATGAACGCGGATACGCTCAAGCCGTCGCCGAACGTATCGGTG | |
| | | CGCCTCTGCGGACCCTTCGTCGAGAGCCGTTCGCGTTCGATGAAAC | |
| | | CGTGCTGGCAGCCGCTGGACAGGCCGCACGTCCGAATTTTAACGCC | |
| | | CTCGATCCTGGATACGATGCCGGGCTCGTGGGTGCCCTGGAAGCTA | |
| | | TCGATGCTCGTGCATTATTTACGGGCCATGGCGGTGATACCGTGTT | |
| | | TTATCAAGTGGCGGCCAGTGCCTTGGCCGCAGACTTACTGGGCGGC | |
| | | GCACCATGTGAAGGTAGCCGCCGTGCACGTTTAGAGGAAGTAGCTC | |
| | | GGCGGACCCGACGCTCGATTTGGAGTCTTGCATGGGAAGCGTTTTC | |
| | | TGGTCGACCCAGCACTGTAAGCATTGAAGGTCAGTTGCTTCGACAG | |
| | | GAAGCAGAGAGAATTCGGCGCGTCGGCCTGACCCATCCGTGGGTTG | |
| | | GAGGCCTGTCGTCTGTGACCCCTGCGAAACGCCAGCAAATCCGCGC | |
| | | GCTGGTCAGTAACCTGAACGCGCATGGCGCCACTGGTCGCGCCGAA | |
| | | CGCGCTAGAATCGTGCACCCGCTTTTAGCTCAGCCGGTGGTTGAAG | |
| | | CCTGCCTGGCGATTCCTGCCCCTATCCTCAGTGCGGGCGAAGGAGA | |
| | | ACGCTCATTTGCGAGAGAAGCCTTTGCAGACCGTTTGCCACCGAGC | |
| | | ATTGTGGGCCGCCGAAGCAAAGGGGAAATTAGTGTGTTTCTTAACA | |
| | | GATCTTTAGCAGCCAGCGCCCCCTTTCTGCGTGGCTTTTTACTTGA | |
| | | AGGACGGCTGGCGGCTCGCGGGCTGATTGATCGTGACGAACTTGCA | |
| | | GCCGCGCTGGAACCGGAAGCAATCGTCTGGAAGGATGCGTCACGCG | |
| | | ACCTGCTTACTGCGGCGGCCCTGGAGGCGTGGGTCAGACATTGGGA | |
| | | AGCACGTATTGGCGAGGGGGAAGCAGCGGAAGGTGAGCGTGCTGCC | |
| | | GGTCGTGGTACCGCAGCGACGGGACCGCGTACAAGCGCGCGGAAGG | |
| | | CGAACACCGGT | |
| epiD | Codon | ATGCACGGTAAACTGCTGATCTGCGCAACTGCTTCGATCAACGTCA | 483 |
| | optimized | TCAATATCAACCATTATATTGTGGAGCTGAAACAGCACTTCGATGA | |
| | | GGTGAATATCCTGTTTTCACCTTCCTCGAAGAACTTTATCAACACC | |
| | | GATGTCCTGAAGCTGTTTTGCGATAATCTGTATGACGAGATCAAAG | |
| | | ATCCGCTGCTGAACCACATCAACATAGTGGAGAACCACGAGTATAT | |
| | | CTTGGTGCTGCCTGCCAGTGCCAATACGATCAACAAAATCGCGAAC | |
| | | GGTATATGCGATAACCTCTTGACGACCGTATGCTTAACCGGGTACC | |
| | | AGAAACTGTTTATCTTTCCGAATATGAACATCCGCATGTGGGGAAA | |
| | | TCCGTTCTTACAGAAAAATATTGACCTGCTTAAAAGCAACGACGTG | |
| | | AAGGTGTATTCCCCCGACATGAACAAATCTTTTGAGATAAGCTCAG | |
| | | GCCGCTACAAAAATAACATCACGATGCCGAATATCGAAAACGTGCT | |
| | | GAATTTTGTCCTGAACAATGAGAAACGCCCGCTGGAT | |
| halM1 | Codon | ATGCGCGAACTCCAAAATGCGCTTTACTTTAGCGAAGTGGTTTTTG | 484 |
| | optimized | GACCGAATCTTGAGAAGATTGTAGGAGAAAAGCGCCTCAATTTTTG | |
| | | GCTCAAACTTATAGGTGAGGACCCGGAAAACCTGAAGGAGTTTCTC | |
| | | TCGAGAAAGGGCAATTCTTTCGAAGAACAAACCTTACCGGAAAAGG | |
| | | AAGCTATCGTTCCGAACCGCTTAGGTGAAGAGGCGCTGGAAAAAGT | |
| | | CCGCGAAGAACTTGAGTTCCTCAATACTTACAGCACTAAACATGTG | |
| | | CGTCGCGTTAAAGAGTTGGGAGTGCAGATCCCTTTCGAAGGGATTC | |
| | | TGCTGCCATTCATTAGCATGTATATCGAAAAATTTCAGCAGCAGCA | |
| | | ACTTCGCAAAAAGATAGGGCCGATTCACGAAGAGATCTGGACGCAG | |
| | | ATTGTTCAAGATATCACCTCCAAATTAAATGCGATTCTGCACCGTA | |
| | | CCCTGATCCTGGAACTGAATGTAGCTCGTGTTACCTCCCAACTTAA | |
| | | AGGTGATACTCCGGAAGAAAGATTCGCCTACTACTCGAAAACCTAT | |
| | | TTAGGCAAACGTGAAGTAACTCACCGTCTGTATAGCGAATATCCGG | |
| | | TGGTTCTGCGGTTGCTGTTCACCACCATTTCACACCACATTTCGTT | |
| | | CATTACGGAAATCCTTGAACGCGTTGCAAATGACCGTGAAGCCATT | |
| | | GAAACCGAATTTTCACCGTGTTCCCCGATTGGTACCCTCGCCTCTC | |
| | | TCCACTTAAACTCGGGAGATGCTCACCATAAACAGCGTACTGTGAC | |
| | | GATTTTGGAATTCTCCTCCTCGCTGAAACTTGTCTACAAACCTCGC | |
| | | TCCCTCAAAGTTGATGGGGTGTTCAACGGTTTACTCGCTTTCCTGA | |
| | | ACGATAGAACGGGGGAAGTCATTAAGGACCAGTATTGCCCTAAGGT | |
| | | GTTACAGCGCGATGGCTACGGCTATGTGGAATTTGTCACTCACCAG | |
| | | TCTTGTCAATCCCTTGAGGAAGTGTCAGACTTCTACGAGAGACTCG | |
| | | GCTCTCTGATGAGTCTGTCCTACGTACTGAATAGTTCTGACTTTCA | |
| | | TTTCGAGAACATTATAGCTCATGGTCCCTATCCTGTCCTGATCGAT | |
| | | CTTGAAACCATCATTCATAATACAGCGGATAGCAGCGAGGAAACGT | |
| | | CTACCGCTATGGATCGCGCGTTCCGTATGTTGAACGATTCGGTGCT | |
| | | GTCCACTGGTATGCTTCCCTCCTCTATTTATTATCGCGATCAGCCG | |
| | | AATATGAAGGGTCTGAACGTCGGAGGTGTGAGCAAATCAGAAGGTC | |
| | | AGAAAACACCGTTCAAAGTTAATCAAATCGCCAATCGCAACACCGA | |
| | | TGAGATGCGTATCGAAAAAGATCACGTTACCCTGAGCAGCCAGAAA | |
| | | AATCTGCCCATTTTTCAGTCTGCCGCAATGGAGAGCGTACATTTCT | |
| | | TAGATCAGATCCAGAAAGGCTTTACCTCCATGTATCAGTGGATCGA | |
| | | GAAGAACAAACAAGAATTTAAAGAACAGGTGCGTAAGTTTGAAGGT | |
| | | GTGCCGGTTCGTGCTGTTCTTCGGAGCACGACTCGCTATACCGAAC | |
| | | TGCTGAAATCTTCCTACCACCCTGACCTGCTCCGCAGCGCGTTGGA | |
| | | CCGTGAAGTACTGCTGAACCGTTTGACTGTTGACTCGGTAATGACC | |
| | | CCGTATCTCAAAGAGATTATTCCACTCGAGGTGGAAGATCTGCTGA | |
| | | ACGGTGACGTGCCATACTTCTACACCCTGCCGGAAGAACGCGCCCT | |
| | | GTATCAGGAAGCGTCTGCGATCAATAGTACGTTCTTTACCACTTCG | |
| | | ATTTTCCATAAGATTGACCAGAAAATCGATAAGCTGGGTATCGAGG | |
| | | ACCATACCCAGCAAATGAAGATCTTACACATGAGTATGCTTGCCTC | |
| | | TAACGCTAACCATTACGCCGATGTTGCCGACTTGGATATTCAGAAA | |
| | | GGACACACCATTAAAAACGAACAGTACGTTGAGATGGCCAAAGACA | |
| | | TCGGTGATTACCTGATGGAGTTATCGGTCGAGGGTGAAAATCAAGG | |
| | | GGAACCAGATCTGTGTTGGATTTCGACCGTCCTGGAAGGGAGCTCT | |
| | | GAAATCATTTGGGACATCAGCCCAGTGGGCGAAGATTTATACAACG | |
| | | GCAGCGCTGGCGTCGCTCTCTTTTATGCGTACCTGTTCAAAATTAC | |
| | | AGGTGAAAAGCGTTACCAAGAGATCGCATACAAAGCCCTGGTTCCG | |
| | | GTTCGCCGCAGTGTGGCCCAATTCCAGCACCATCCGAATTGGAGCA | |
| | | TTGGTGCGTTTAACGGAGCGTCAGGCTATCTGTACGCGATGGGTAC | |
| | | GATAGCGGCCCTGTTTAATGATGAACGTTTGAAGCATGAAGTAACC | |
| | | CGCAGCATTCCGCACATTGAACCGATGATCCACGAGGATAAGATCT | |
| | | ATGATTTCATTGGCGGTTCCGCAGGGGCGCTGAAGGTGTTCCTGAG | |
| | | CCTGTCGGGGCTGTTTGACGAGCCGAAGTTTTTGGAACTTGCCATT | |
| | | GCATGCAGCGAACATCTGATGAAAAACGCCATTAAAACGGATCAAG | |
| | | GTATCGGCTGGAAACCACCGTGGGAGGTCACCCCACTGACCGGTTT | |
| | | CAGCCATGGGGTTAGCGGCGTCATGGCATCCTTCATCGAACTGTAC | |
| | | CAGCAAACCGGTGATGAGCGCTTGCTCAGTTACATTGATCAGAGTT | |
| | | TAGCCTATGAACGTTCCTTCTTCAGCGAACAAGAGGAGAACTGGCT | |
| | | GACTCCGAACAAAGAAACACCCGTGGTAGCTTGGTGCCACGGCGCG | |
| | | CCGGGAATTTTGGTATCACGACTGCTTCTGAAGAAATGCGGCTATT | |
| | | TGGATGAAAAAGTCGAAAAAGAAATTGAGGTGGCATTATCCACAAC | |
| | | TATCCGTAAAGGCCTTGGTAACAATCGCAGTCTTTGCCATGGTGAT | |
| | | TTCGGCCAGCTGGAAATTCTTCGCTTTGCGGCGGAAGTGTTAGGCG | |
| | | ATAGCTATCTCCAGGAAGTTGTCAACAATCTGTCCGGCGAGTTGTA | |
| | | TAATCTTTTCAAAACGGAGGGATATCAGAGCGGAACCAGCCGCGGT | |
| | | ACTGAATCCGTGGGCCTGATGGTAGGTCTGTCCGGGTTTGGGTATG | |
| | | GTTTACTTTCAGCGGCATATCCATCTGCTGTCCCCTCAATCTTAAC | |
| | | ATTGGATGGTGAGATCCAGAAGTACCGGGAGCCTCATGAAGCC | |
| halM2 | Codon | ATGAAAACGCCGCTGACCTCGGAACATCCTTCAGTGCCGACGACGC | 485 |
| | optimized | TGCCGCATACTAACGACACCGATTGGCTCGAGCAATTACATGACAT | |
| | | TTTGTCCATTCCTGTTACGGAAGAAATCCAGAAATATTTCCACGCC | |
| | | GAAAATGATCTGTTCTCGTTTTTCTATACACCGTTCCTGCAGTTTA | |
| | | CGTACCAGAGCATGTCGGACTACTTTATGACCTTCAAGACCGATAT | |
| | | GGCCCTGATCGAAAGACAGAGCCTCCTGCAAAGCACGCTGACCGCG | |
| | | GTACATCACCGACTCTTCCACTTAACGCATCGCACCCTTATTAGTG | |
| | | AAATGCATATTGATAAACTTACCGTTGGCCTGAATGGCTCTACGCC | |
| | | GCACGAGCGCTACATGGATTTCAACCACAAATTCAACAAAACCTCG | |
| | | AAGTCGAAGAACCTGTTTAACATCTACCCAATTTTGGGAAAATTGG | |
| | | TCGTTAACGAAACTCTGCGCACTATTAACTTCGTCAAGAAAATCAT | |
| | | TCAGCACTACATGAAGGACTACCTGCTCCTGTCGGACTTCTTCAAA | |
| | | GAGAAGGACTTGCGTCTTACCAACCTGCAATTAGGCGTGGGGGATA | |
| | | CACACGTTAATGGGCAATGCGTCACCATTCTGACGTTTGCATCAGG | |
| | | CCAAAAAGTGGTATACAAACCTAGATCATTGTCGATAGATAAACAG | |
| | | TTCGGAGAATTCATCGAGTGGGTAAACTCGAAAGGTTTTCAGCCTT | |
| | | CCTTGCGTATCCCTATTGCGATTGATCGTCAAACCTATGGTTGGTA | |
| | | TGAATTCATCCCTCATCAAGAGGCCACCAGCGAAGATGAAATAGAA | |
| | | CGCTACTATTCTCGCATCGGTGGTTATCTGGCGATCGCCTACTTGT | |
| | | TCGGGGCAACCGACCTGCACCTGGATAACCTGATCGCCTGCGGCGA | |
| | | ACATCCGATGCTTATTGATTTGGAAACACTCTTTACCAACGATCTC | |
| | | GACTGCTATGACAGTGCGTTTCCGTTCCCGGCGCTGGCCCGCGAAT | |
| | | TAACCCAATCCGTTTTTGGCACCCTTATGCTTCCCATCACCATCGC | |
| | | GTCGGGGAAACTGCTGGATATAGACCTGTCAGCAGTAGGAGGCGGT | |
| | | AAAGGTGTGCAGTCCGAAAAGATCAAAACCTGGGTCATCGTGAATC | |
| | | AGAAAACTGATGAGATGAAGCTGGTCGAGCAGCCGTATGTTACCGA | |
| | | GAGTTCCCAGAATAAACCAACAGTTAATGGGAAAGAGGCGAACATT | |
| | | GGCAATTATATTCCTCATGTCACAGATGGCTTTCGTAAAATGTACC | |
| | | GCCTGTTTCTGAATGAAATTGATGAGTTAATGGATCATAACGGGCC | |
| | | AATCTTTGCGTTTGAGAGTTGTCAGATTCGTCATGTTTTTCGAGCT | |
| | | ACCCACGTGTATGCGAAATTTTTGGAGGCAAGTACCCACCCAGATT | |
| | | ACTTGCAAGAACCTACCAGACGTAATAAACTGTTCGAGTCCTTTTG | |
| | | GAACATCACGTCGCTGATGGCACCGTTCAAGAAAATTGTACCGCAC | |
| | | GAAATCGCGGAGTTGGAGAACCATGATATTCCGTACTTCGTCCTGA | |
| | | CTTGTGGCGGCACCATTGTTAAAGATGGATACGGCCGGGATATCGC | |
| | | AGACCTGTTTCAAAGTAGCTGCATCGAACGTGTAACTCATCGTCTG | |
| | | CAGCAGCTGGGAAGCGAGGATGAGGCGCGTCAAATTCGCTACATTA | |
| | | AAAGCAGCCTGGCGACGTTGACCAACGGTGATTGGACCCCATCCCA | |
| | | TGAGAAAACCCCGATGTCTCCGGCCTCGGCCGACCGTGAAGATGGT | |
| | | TACTTCCTGCGCGAGGCTCAGGCCATCGGCGACGACATTTTGGCGC | |
| | | AGCTGATTTGGGAGGATGACCGTCACGCCGCTTACCTTATTGGCGT | |
| | | AAGCGTGGGCATGAACGAAGCCGTCACTGTGTCACCCCTGACGCCT | |
| | | GGCATCTACGACGGCACACTTGGCATAGTGCTGTTCTTCGATCAGC | |
| | | TGGCCCAGCAGACCGGCGAAACCCATTATCGCCACGCCGCCGACGC | |
| | | TTTACTGGAAGGAATGTTCAAACAGCTGAAACCTGAACTGATGCCG | |
| | | TCTAGCGCTTACTTCGGACTGGGTAGCCTGTTCTATGGCCTGATGG | |
| | | TGTTGGGCCTCCAGCGTTCCGACTCGCATATCATTCAGAAAGCGTA | |
| | | TGAGTATCTGAAACATTTGGAAGAGTGTGTGCAGCATGAGGAAACG | |
| | | CCAGATTTTGTCTCGGGTTTGTCTGGTGTACTGTATATGCTCACGA | |
| | | AAATTTATCAGCTCACGAATGAACCGAGAGTTTTCGAAGTGGCCAA | |
| | | AACCACAGCTTCGCGTCTGTCTGTGCTGCTTGACAGCAAGCAGCCC | |
| | | GACACTGTGCTCACCGGGTTATCCCATGGCGCCGCAGGATTCGCCC | |
| | | TTGCATTACTGACCTACGGAACCGCTGCAAATGATGAACAGTTGCT | |
| | | GAAACAGGGCCACTCCTATCTGGTGTACGAACGTAATCGGTTTAAC | |
| | | AAACAGGAAAACAACTGGGTTGATTTACGTAAAGGCAACGCGTATC | |
| | | AAACATTTTGGTGCCATGGCGCCCCGGGTATTGGCATCTCACGCCT | |
| | | CCTGTTAGCGCAATTTTACGATGACGAACTGCTGCATGAAGAGTTA | |
| | | AACGCAGCACTGAACAAGACTATTTCGGACGGCTTCGGCCACAATC | |
| | | ACTCACTGTGTCATGGCGATTTCGGCAACCTCGATCTGTTATTGCT | |
| | | TTATGCCCAATATACGAATAACCCAGAACCAAAGGAACTCGCTCGC | |
| | | AAACTGGCCATAAGCAGTATCGATCAAGCGCACACGTATGGCTGGA | |
| | | AACTCGGGCTCAATCATAGCGATCAACTGCAGGGTATGATGTTAGG | |
| | | GGTGACTGGTATCGGCTATCAGCTCCTTCGTCATATAAATCCGACA | |
| | | GTCCCCAGCATTTTGGCACTGGAACTGCCCAGCTCCACGTTAACTG | |
| | | AAAAAGAGCTGAGAATCCATGATCGT | |
| kgpF | Codon | ATGATCAATTATGCTAATGCGCAGCTCCATAAGAGTAAAAACTTGA | 486 |
| | optimized | TGTATATGAAAGCCCACGAAAACATCTTCGAAATCGAGGCGCTGTA | |
| | | CCCGCTGGAATTGTTCGAGCGTTTTATGCAGTCCCAAACCGATTGC | |
| | | TCCATCGATTGTGCCTGTAAAATTGATGGTGACGAATTGTATCCCG | |
| | | CCCGTTTTAGTCTGGCCCTGTATAACAACCAGTATGCCGAAAAGCA | |
| | | AATTCGCGAAACCATCGACTTCTTCCATCAGGTAGAGGGTCGGACC | |
| | | GAGGTGAAACTGAACTATCAGCAACTGCAGCACTTCCTGGGTGCTG | |
| | | ACTTCGATTTTAGCAAAGTGATTCGAAACCTGGTGGGTGTGGATGC | |
| | | ACGCCGCGAACTGGCTGATTCCCGGGTTAAACTGTATATTTGGATG | |
| | | AACGATTACCCAGAGAAAATGGCGACCGCCATGGCATGGTGCGATG | |
| | | ATAAGAAGGAATTGTCGACGTTGATAGTAAATCAGGAGTTTCTGGT | |
| | | CGGGTTCGATTTTTATTTCGATGGTCGCACGGCAATAGAATTATAC | |
| | | ATTAGTCTGTCATCCGAAGAATTTCAGCAGACACAAGTTTGGGAAC | |
| | | GCCTCGCAAAGGTAGTGTGCGCCCCAGCGCTGCGCCTTGTTAATGA | |
| | | TTGCCAGGCGATCCAGATTGGCGTGAGCCGTGCCAATGATAGTAAG | |
| | | ATCATGTATTACCATACCCTTAATCCGAACTCGTTTATCGACAATC | |
| | | TGGGCAATGAAATGGCAAGCAGAGTTCACGCGTATTACCGACATCA | |
| | | ACCGGTTCGCTCTCTGGTAGTATGCATACCAGAACAGGAGTTGACC | |
| | | GCCCGGTCCATACAGCGCTTAAACATGTATTACTGTATGAAC | |
| lasB | Codon | ATGAAAGGCGAGGAAATGTTGGGACATCCACAGACCGGTTTTGTTG | 487 |
| | optimized | TACTGCCAGACAACGATGCCACCGGCGACGTGACGGGCCGCCTGTT | |
| | | ACCTTGGGGTGATGTAGTTACAGTGTATCCGTCTGGCCGTCCATGG | |
| | | ATCATCGGCAACTGCTGGGATCGCCCAGTCCTCGTCCATGATGGCG | |
| | | TGATCGTCTTGGGTCATACCAGCGTCACGCGTGATCAAATTGCCCG | |
| | | TCATGGGAACGATCCGCATCGCTTACTGGACGAGGCCGACGGCGCA | |
| | | TTTCATGCGGCGGTCCTGATCGGACACGAAGTTCATGTTCGCGGCT | |
| | | CCGCCTACGGTGTCTGTCGTCTGTATACATGCGTTGTTGACGGTGT | |
| | | GACCTTAGTGAGTGATCGTACAGACGTCCTGCAGCGTCTGGCAGGT | |
| | | ACTGATGTGGACGTCGACGTGCTGGCTGGCCACTTGTTAGAGCCGA | |
| | | TCCCGCACTGGTTAGGCGAACAACCGTTATTGACGTCCGTGGAGCC | |
| | | CGTGCCACCGACACATCACGTTATTTTAACTCCGGACGCACGTAGT | |
| | | CGTTTACGGCCATCACGTCGTCGTCGGCCTGAACCGTCGCTGGGTT | |
| | | TGCGGGACGGTGCGGAACTTGTCCGGGAGCGTCTGGCCGCAGCTGT | |
| | | GGCTACCCGTGTGGACAGTCCAGCGTTAATTACCAGTGAACTGAGT | |
| | | GGCGGCTATGATTCCACTAGTGTGTCATACTTGGCAGCGCGCGGTA | |
| | | AAGCCGAGGTGGTGCTGGTCACGGCCGCGGGACGTGACAGCACAAG | |
| | | CGAGGATCTGTGGTGGGCTGAACGCGCAGCCGCAGGGCTCCCGGAA | |
| | | CTCGATCACGTAGTGTTACCTGCGGATGAATTACCGTTTACGTACG | |
| | | CCGGCCTGACGGAGCCTGGTGCACTTTTGGATGAACCGTGTACGGC | |
| | | TGTTGCCGGCCGTGAGCGTGTACTGGCGCTGGTACGTAAAGCCGCG | |
| | | GCCCGCGGCTCTACACTTCATCTGACTGGCCATGGTGGCGATCACC | |
| | | TGTTTACTTCACTGCCGACACCGTTTCATGACCTGTTTCGTACGCG | |
| | | TCCAGTCGCCGCGCTCCGCCAGTTGCGTGCATTTGGCGCGTTGGCT | |
| | | GCGTGGCCGACCCGTAAGCTGATGCGCGAACTCGCGGACCGCCGCG | |
| | | ATCATAGCACCTGGTGGCGCGCGCACGCACGTCCTCAGAATGGCCA | |
| | | GCCGGATCCGCACAGCCCCATGTTAGGCTGGGCAATTCCCCCGACT | |
| | | GTCCCGGCGTGGGTTACTGCTGACGGCGTGCGCGCGATCGAACTTG | |
| | | GGATTTTAGAAATGGCAGAACGCGCGGAGCCCCTTGGTCATGCGCG | |
| | | CGGAGAACACGCTGAGCTGGATTCAATCTTTGAAGGGGCGCGTATG | |
| | | GCCCGTGGCCTCAATCGTATGGCTACGCATGCCGGAGTCCCGCTTG | |
| | | CAGCCCCGTTCCATGACGATCGGGTCGTGGAAGCGTGTCTGTCGAT | |
| | | CCGGCCGGAGGAACGCATTTCTGCATGGCAGTACAAACCCTTACTG | |
| | | AACGCCGCAATGCAGGGTGTGGTGCCGAGCACCGTTCTTGATCGTA | |
| | | GCGCTAAAGATGACGGGAGTATTGATGTGGCCTATGGGCTGCAGGA | |
| | | ACACCGTGATGAACTGGTAGCGCTGTGGGAATCATCACGTCTGGCG | |
| | | GAAACCGGTCTGATTGATGCGGGTATGCTGCGGCGTTTATGCGCGC | |
| | | AGCCGTCCTCCCACGAGCTCGAGCATGGATCCTTGTACGCTACTAT | |
| | | CGCTTGTGAGTTGTGGCTGCGTGGTTTAGATCAGGATCGTACCCAA | |
| | | CGCTAC | |
| lasC | Codon | ATGCCGGTGCAGCTGCGTCGGCATGTGTCTTTTACGGCTACGGAAT | 488 |
| | optimized | ACGGCGGCGTGCTGCTGGATGAAACCAAAGGCGCATACTGGCGTCT | |
| | | GAACACCACAGGCGCCGAAGTTGTTCGCGCCATGGGGGAAGCCGAG | |
| | | CGGGATGAGATTGTACGGCATGTGGTGGCGACCTTCGATGTTGATG | |
| | | CGCAAACCGCAGCCCAGGATGTCGATGTCCTGCTGGCAGAACTTCG | |
| | | TGATGCCGGCCTTGTGGCCTCG | |
| lasD | Codon | ATGTCTGTGAATATGGCTCTCCGTGGCCATGGTATGTCCGGTCGCC | 489 |
| | optimized | GTCGTCGCTTAGATGCCACGCGTGCTCGCCTGGCCGTTGTGGTTGC | |
| | | CCGTGTCCTGAATCTCTTACCGCCGCGCTTAATCCGTCGTTGTTTG | |
| | | CGTGTACTGAGTCGCGGAGCCCGCCCTGCCTCGATTGAGGCAGCAG | |
| | | AAGCTGCTCGTCGTACTGTGGTTGCGGTGAGTCCAGCTGCCGCCGG | |
| | | TGCGTACGGCTGTTTAATCCGCAGCATTGCCACCACCCTGGTTCTT | |
| | | CGTTCACGCGGGCAATGGCCAACCTGGTGTGTTGGTGTACGTGCGG | |
| | | AGCCTCCTTTTGGTGCCCATGCCTGGATTGAAGCAGAGGAGCGGCT | |
| | | GGTGGATGAACCTGGTACTATGCATACTTACCGTCGTCTTATCACC | |
| | | GTTGGTCCACTGTCTCGCAAAGTTCGT | |
| lasF | Codon | ATGTCTATCGAACTGACGCCTAGTTTGGCCGATCTGGTCGATCCAC | 490 |
| | optimized | TTCCAGGTCACGCACTGCGCGCTGCGGCGACATTACGTCTGGCAGA | |
| | | TCTGATTGCGGCTGGTGCAGATACTGCACCGGCATTAGCAGCGGCG | |
| | | GCACGCATTGATGCTGACGCGATCGCGCGTCTTATGCGGTATCTGT | |
| | | GCAGTCGCGGGATTTTTCAAGCACATGAAGGCCGGTACGCGTTGAC | |
| | | TGAATTTAGCGAATTGCTGCTGGATGAAGATCCATCTGGCCTGCGT | |
| | | AAAACCTTAGATCAGGATAGCTATGGGGATCGTTTCGACCGCGCGG | |
| | | TTGCGGAACTGGTGGACGTTGTACGGTCCGGTGAACCTTCTTATCC | |
| | | TCGCCTTTACGGCTCGACGGTTTATGATGACCTGGCAGCCGATCCT | |
| | | GCCCTCGGCGAGGTGTTCGCGGATGTTCGTGGCTTGCACTCCGCAG | |
| | | GGTATGGGGAAGATGTCGCGGCAGTGGCGGGTTGGTCCTCATGCCT | |
| | | GCGCGTTGTCGATCTGGGTGGAGGGACTGGCTCCGTCCTGCTTGCT | |
| | | GTGTTAGAGCGTCACCCGTCCCTGTCAGGCGCAGTACTGGATCTGC | |
| | | CATACGTCGCCCCGCAGGCAAAGAAAGCTCTGCAGGCCTCAGCGTT | |
| | | TGCCCAACGTTGTGAATTTATCAAAGGGAGCTTCTTCGATCCGTTA | |
| | | CCTCCGGCAGACCGTTACCTGTTGTGTAACGTGCTGTTCAACTGGG | |
| | | ATGACGCGCAAGCAGGCGCTATTTTGGCACGCTGTGCGCAGGCGGG | |
| | | CCCTGTGGCCGGAGTAGTGGTAGCCGAACGTTTGATCGATCCGGAT | |
| | | GCGGAAGTGGAACTCGTAGCAGCTCAAGATCTGCGTCTGTTGGCTG | |
| | | TTTGCGGCGGTCGGCAGCGTGGCACCGCTGAATTCGAAGCGCTTGG | |
| | | GGCAGCCCATGGCCTGGCGTTAACCAGCGTTACCCTCACGGCATCT | |
| | | GGTATGAGCCTGCTCCGTTTCGATGTGTGTCGTGCCGGGAGTGCTG | |
| | | GCGGGGAAGTTGTGGAAAAATCT | |
| IcnG | Codon | ATGGACGGAACCAACAAGCGCCTGGAGGACAAGTGGTTTGATATTA | 491 |
| | optimized | ACTTCCTGGAAATGTATACACGCAGCTGCCTGAAAACTTTTGGCTA | |
| | | CTTCGACGAAATTCTGATCGTGAAGAAACGCATCGAGGTCCTGAAG | |
| | | AACGTGCTTGAAAAACAGTACTTGTCTACCAATGATTATGCTGAGG | |
| | | AGTTTTTCGAGCTGAATACCACCTTGGAGAGCATAAAAGAATACAT | |
| | | CAAACTGAATCTGGTCATCGAGAAAGAACCGATCTCAATTTGCATT | |
| | | ATGGTCAAAAACGAAGAACGTTGCATCAAGCGCTGCATTGATAGCG | |
| | | TTGAAATCCTCGCCGAGGAGATAATCATTATCGATACCGGCTCTAC | |
| | | GGATAATACCATTAACATTATTGAGGAATGCGCAAACGACAAAATT | |
| | | AAAGTGTTCTCAAAAGAATGGCGTAACGATTTTTCCGAAATTCGGA | |
| | | ACTATGCCATCGAGAAAGCGAGTAGCGAATGGCTGGTGTTTATAGA | |
| | | TGCCGATGAATATCTGGACGAAGCCTCGGTGCTCAACCTGCTCAGT | |
| | | ACGCTCAACATCTTTAACAATCATAAGCTCAAAGACTCTATTGTCC | |
| | | TGTGCCCCATGATCAACGAAGCCAATAACACCATCCATTTCCGTAC | |
| | | CGGGAAATTTTTCAGAAAAGACTCCGGGATTAAATTCTTTGGTACC | |
| | | TGCCATGAGGAGCCCCGCATTAAAGGCATGCCGAATTCTACCCTGC | |
| | | TGATTCCGATCAAGGTTGATTATCTGCATGACGGCTACCTGGCAAA | |
| | | AGTACAATCAAATAAAGACAAGAAAACCCGTAACATCGAACTGTTA | |
| | | GAAGGTATGGTGGAACTGGAACCGGATAATCCTCGTTGGGCGTATA | |
| | | TGTTTGTGCGCGACGGATTTGCAATCCTCGATAACGAATACATTGA | |
| | | GAAAACTTGTTTGCGGTTTTTACTGCTGGACAAAAACGTACGCATC | |
| | | TGCGTCAACAACCTGCAAGACCATAAATTCACTTTGTCACTCCTGA | |
| | | CGATCCTGGGCCGCCTCTATCTGCGCGAGTGCGAATTCGAGAAAAG | |
| | | CAATCTGATAATTCGCATTCTTGACGAACTCATCCCTAATAGTCTG | |
| | | GATGGTAAATTTCTGGCATTCATGGAGCGATTCAGCAAACTGAAAA | |
| | | TTGAGATTAATACGCTGTTAACGGAGGTCATCGAATATCGTCGTAA | |
| | | CCACGAAGTAGATGAAACCAGTTTAATCAACACACAAGGCTACCAT | |
| | | ATCGACTATGTTCTGTCGATTTTGCTGTTCGAAACGGGTAATTACG | |
| | | CGCAAAGTAAGAAATACTTCGATTTCCTGCAGGAGAACCATTTTCT | |
| | | GGAAGAACTGTTTCAAGACAGCTCTTATTCTATCATACTGAAAATG | |
| | | CTCGAGTCAGTAGAAGAT | |
| ItnMI | Codon | ATGAAGTTTAACAAGAACGTGTTCCCAGAGATCAATGAAACGGATT | 492 |
| | optimized | TCGATAACAATATCAAGCCCCTGCTGGATGAACTGGAATCTCGTAT | |
| | | TACCATTCCGCAGGAGGAACTGAGCTTTTCAAGCATTAACGATGAT | |
| | | TTATTTCGCGAGTTAACCCGCAACGAGGAGTACCCTTACCAGAGCA | |
| | | TTTGTACGATCGTTGCAAACATCGTGATGGATGACGGCAGTGAGAT | |
| | | TTGGCGCAAAGATATTTTTGTTGATTCCAATAGTGTGCGCGAAGCC | |
| | | GTATGCGACATTCTGAGCCAAACGTTATTCCTCTATTTCATCCGCT | |
| | | GCTTCTCCGAACAAATTAAAGACATTCGCAAAACTGATGAGGATAA | |
| | | AGAGTCCACCTACAACCGCTACATTAACCTCCTGTTCAGCTCCAAC | |
| | | TTCAAAATCTTCTCCGACGAATACCCTGTCCTGTGGTATCGGACCA | |
| | | TTCGCATCATCAAAAATCGCTGGTATTCTATCAAGAAATCGTTACT | |
| | | GCTGACTCAAAAACACCGTGTGGAGATCGATAAGCAGTTGGACATC | |
| | | CCGCACAAGATGAAGATTAAAGGCCTGAAAATCGGGGGAGACACGC | |
| | | ATAACGGCGGTGCCACAGTGACCACGATCTTCTTTGAGAAAGGGTA | |
| | | TAAACTGATTTATAAGCCGCGGAGCACATCCGGCGAATTCTCGTAC | |
| | | AAGAAATTTATCGAAAAGATTAACCCGTACCTGAAGAAAGACATGG | |
| | | GAGCGATTAAAGCGATCGATTTCGGTGAATACGGCTTTTCTGAGTA | |
| | | TATTGAGTGTAACACGGATGAAGAGGACATGAAACAGGTCGGTCAG | |
| | | CTTGCATTTTTCATGTACCTGTTGAATGCATCAGATATGCATTATA | |
| | | GCAATGTCATTTGGACCAAACAGGGCCCTGTGCCGATTGATTTAGA | |
| | | AACCTTGTTCCAGCCGGATCGTATTCGCAAAGGCCTGAAGCAGTCG | |
| | | GAAACTAACGCGTACCACAAAATGGAGAAAAGTGTATACGGAACGG | |
| | | GAATTATTCCAATTTCCCTGAGCGTTAAAGGCAAAAAGGGTGAGGT | |
| | | CGACGTCGGCTTTAGTGGAATCCGTGATGAGCGCTCTAGTTCGCCG | |
| | | TTTCGCGTTCTGGAAATTTTGGATGGGTTTTCGAGCGACATCAAAA | |
| | | TCGTGTGGAAAAAGCAGCAGAAGTCTAGCTCCAGCAAAAACAATCT | |
| | | GATTGTCGATCACAAAAAGGAGCGCGAAATCCTTCAGCGTGCCCAG | |
| | | TCCGTCGTAGAAGGTTTCCAGGAAACCTCTAAAATCTTCATGAAAC | |
| | | ATCGTGAGGAATTCATCTCCATTATCTTAGACTCATTCGAGAACAT | |
| | | CAAAATTCGCTACATCCATAACATGACGTTTCGCTACGAACAGTTG | |
| | | CTGCGCACTCTGACGGATGCCGAGCCGGCCCAGAAGATTGAGTTAG | |
| | | ACCGTCTGCTGCTGAGTCGTACCGGAATTCTGTCCATCTCGTCTAG | |
| | | TCCCTACATCTCGCTCTCCGAATGTCAACAGATGTGGCAGGGTGAC | |
| | | GTGCCGTACTTCTACTCGAAGTTTTCGAGCAAAAGTATCTTTGATA | |
| | | CCAATGGCTTCGTTGATGAAATCGAGCTGACGCCCCGCCAGGCATT | |
| | | TATCATCAAAGCCGAAAGTATCACCAACGATGAAGTCGATTTTCAG | |
| | | TCCAAGATCATTAAACTGGCGTTCATGGCACGCTTAAGTGACCCGC | |
| | | ACACAACCAACGACAACAAACTGAATAAAAAGGTGATTATCGAAAG | |
| | | CAACCAGCAGAGCAACAGCAGTGAATCAGGTAACAAAGCCATTTTG | |
| | | TTCCTGAGCGATCTGCTGAAAAATAACGTACTGGAAGATCGTTATA | |
| | | GTCATCTGCCGAAAACTTGGATTGGCCCTGTAGCACGTGATGGCGG | |
| | | TTTGGGTTGGGCGCCGGGCGTGCTGGGATACGATCTGTACTCGGGC | |
| | | CGTACAGGACCTGCGTTAGCATTGGCTGCGGCCGGGCGCGTTTTGA | |
| | | AAGATAAAGACAGTATCGAACTTAGCGCCGACATTTTTAATAAATC | |
| | | GTCCCAGATTCTGCAGGAAAAGACTTACGACTTTCGTAACCTGTTC | |
| | | GCATCAGGTATCGGCGGTTTTAGCGGGATTACCGGTCTGTTTTGGG | |
| | | CGCTGAACGCGGCAGGGAATATTCTGAACAATGATGACTGGATTAA | |
| | | AACCTCGAATCAGAGTATGCTGCTGCTGAATGAGAACATGCTGAAA | |
| | | GTGGACAAAAATTTCTTTGACCTGATTAGCGGCAACTCGGGAGCGA | |
| | | TCGGTATGATGTACCTGACCAATCCAAATTTCTATTTGTCTCGCTC | |
| | | GAAAATTAACGACATTCTGCTGACCACGGACTGCTTGATTACTGAA | |
| | | ATGGAAAAAGACGAAACGAGCGGACTGGCCCATGGCGTGTCTCAGA | |
| | | TCCTGTGGTTCCTTAGCATTATGATGCAACGTCAGCCCTCAAGTGA | |
| | | AATCAAAATCCGCGCGACGATTGTCGACAACATCATCAAGAAGAAG | |
| | | TATACGAATTCCTATGGCGAAATCGAATGCTACTATCCGACTGATG | |
| | | GGCACTCCAAATCCACCTCGTGGTGCAACGGGACAAGTGGGATTCT | |
| | | GGTCGCCTATATTGAGGGGTATAAAGCTAATATCGTGGACAAATCC | |
| | | TCGGTGTATCATATTATTAATCAGATCAACGTCGAACAACTTCAGC | |
| | | ATGATAACATTCCGATCATGTGCCATGGTAGCCTTGGTGTGTATGA | |
| | | ATCGCTTAAATATGCGTCAAAGTACTTTGAAATCGAAACCAAGTAC | |
| | | CTTCTGGATGTGATGCGCAATGGCGGCTGCTCCTCCCAAGAAGTAT | |
| | | TAAAGTACTATGGCAAGGGTAACGGCCGTTACCCGCTGTCACCAGG | |
| | | TTTAATGGCGGGTCAGTCGGGCGCGTTGCTGCACTGTTGCAAACTG | |
| | | GAGGATAACGATATCAGCGTGAGCCCCATTTCACTGATGACG | |
| ltnM2 | Codon | ATGGATCCGAGTATCAAAAAGCTCGTGGATTCTATCATCGAATTCT | 493 |
| | optimized | ACAAAAAGGACATCTACCTGGCATACAAAGAGCTGGAACGCGAAAT | |
| | | CAAAAACATCGATAAGACCATCTACAACACTTCAAATGACGAGATC | |
| | | TTGCGGATTTTTAAAGAGAGCCTGATCAGCATCATCACCGATGATA | |
| | | TTTACCGCCTCTCGATTAAAACCTTCATCTATGAGTTTCACAAGTT | |
| | | TCGTATCGATAACGGGTTTCCGGCTGTCAAAGATAGCGAAAGCGCC | |
| | | TTCAATTATTACATCAGTACCTTTGACGTGAAAACGATCGCTCGCT | |
| | | GGTTTGAGAAATTCCCAATGCTGGAATCCATCATCTCCAGTAGCAT | |
| | | CAAAAACGATTGCACATTTATGGTGGATGTATGTGTCAATTTCATC | |
| | | TTAGACCTGTCGGAATGCGAGAAGATTAATCTGATCTCAGAGGATA | |
| | | GCCGGCTCATCACGATCTCATCCAGCAACTCTGACCCGCACAACGG | |
| | | TGGCACGCGTGTCTTGTTCTTTCGTTTCCACAACGGTGATACCATT | |
| | | CTTTACAAACCCCGCAGCCTGACCGTGGACAAGCTGATCTCTAATA | |
| | | TTTTCGAAGAGGTATTCGAATTCGATGCGACGAACTCGAAAAATCC | |
| | | TATTCCCAAGGTGCTGGATCGGGGTACCTATGGCTGGCAGGAATTC | |
| | | ATTGAGAAGAAATCGATCTCTTCCTCAGAGATTAAGCAGGCCTACT | |
| | | ATAACCTGGGTATCTTTAGCAGTATCTTTACAGTGTTAGGGTCTAC | |
| | | TGATATCCACGATGAAAACTTGATTTTTAAAGGTACGACCCCGTAT | |
| | | TTCATCGATCTGGAAACAGCCCTCTCTCCGCGTATCCGGTATGAAG | |
| | | GTAATGAGGAAAACCTGTTCTATCGGATGAGCTCATCGTTGTTCAC | |
| | | TTCTATCGTGGGGACGACTATTATTCCTGCAAAACTTGCTGTCCAT | |
| | | TCCCAGGAAATTATGATCGGCGCAATTAACACCCCTGCGAAACAGA | |
| | | AAACCAAGAAGGATGGCTTTAACATCATCAACTTCGGCACGGATGC | |
| | | CGTCGATATCGCAAAACAGAATATTGAGGTGGAGCGTATTGCTAAC | |
| | | CCTATGCGCATTAAAAATAACATCGTGAACGATCCGCTGCCGTACC | |
| | | AGAACATCTTTACGCGCGGCTTCAAAGAGGGGATCAAATCCATCAT | |
| | | CCTGAAGAAAGGCTCGATCATTTCCATTCTGAACAACTTCAACAGC | |
| | | CCGATTCGTTACATCATGCGGCCGACGGCAAAATATTATTTGATTC | |
| | | TGGATGCCGCGGTATTTCCCGAAAACCTGTATTCGGAACAGACACT | |
| | | GAACAAAACCCTGAATTAGTTAAAGCCGCCAAAAATCGTGGAAAAT | |
| | | TCCCTGATTTCTAAACAGCTCTTTCTTGCCGAAAAACGCATTCTGT | |
| | | CCGAAGGCGATATTCCGAGCTTCTATGTGCTGGGCAAAGAGAAAAA | |
| | | TATCCGTGCGCAGAACTTCATTAGCGAACAGATCTTCGAGGAAACC | |
| | | GCGGTCGATAACGCGATTCAAATTCTGGAATCCATTTCGCAAGACT | |
| | | GGGTGAATTTTAATGAGCGCCTGATTGCGGAGGGCTTCTCCTATAT | |
| | | TCGTGAACAGAGTCGTGGCTATCTGTCCAGTGATTTTGAGAACTCT | |
| | | GATATTTTCAAAAGCTCACTGACCGAAACAAAGAAGTCCGGTTATA | |
| | | CCGCAATGCTGAAAACAATTATCTCCATGTCGGTCAAGACCTCGGA | |
| | | AAACAAAAAGATCGGTTGGCTGCCAGGCATTTATGATGATTATCCG | |
| | | ATCAGCTATATGAGTGCCGCGTTTTGTTCGTTCCATGATTCCGGCG | |
| | | GTATCATCACTTTGCTTGAACACCACTTTGGGCACTGCTCCCCCGA | |
| | | ATATAACGAGATGAAGCGCGGGCTGCTGGAACTGGGCAAAATGTTG | |
| | | AAAATTAACAATAGTAACCTGAGCATCATCTCCGGCTCAGAGTCTC | |
| | | TGGAATTTCTGTATACGCACCGCGAAGTCGAATGCCTGGAACTGGA | |
| | | ATACATTTTAAACAATTCAGCGGAAATCATGGGCGACGTGTTCCTG | |
| | | GGGAAATTAGGCCTTTATCTTATCCTGGCGAGCTACCTGAAAACAG | |
| | | ACCTGAAAATTTTCCAAGATTTCAGTATCATCTGCCAGAAAAACCT | |
| | | CGAGTTTAAAAAGTTCGGGATCGCGCACGGTGAATTAGGGTATCTG | |
| | | TGGACCATCTTCCGTATTCAAAACAAACTGAAGAACAAAAATGCGT | |
| | | GTCTGAGCATCTATCATGAAGTGTTGAACATTTATAAAGGTAAGCG | |
| | | CATTGAATCCGTGGGATGGTGCAACGGTTTATCGGGTATTCTGATG | |
| | | ATTTTGTCAGAAATGAGCACCGTATTAGAGAAAAATCAAGACTATC | |
| | | TGTTCAAGCTGGCAAATCTGAGCACTAAACTGAATGAGGAATCCGT | |
| | | TGACCTGAGTGTGTGCCACGGCGCCAGCGGGGTGCTTCAAACACTG | |
| | | CTTTTCGTCTATAGCAACACGAACGATAAACGTTATCTCAGCCTGG | |
| | | CCAATAAGTATTGGAAGAAAGTGCTGGATAACAGCATTAAGTACGG | |
| | | TTTCTACAATGGAGAACGCGATAAGGATTATCTGTTGGGATATTTC | |
| | | CAGGGTTGGTCAGGCTTCACGGACAGCGCACTCCTGCTGGATAAAT | |
| | | ACAATAACAATGAGCAAGTGTGGATTCCGATCAACCTGAGCTCCGA | |
| | | TATCTATCAGCATAATCTGAACAACTGCAAAGAGAAGAATTATGAG | |
| | | GGCGATGGCTGCCATAAATCT | |
| lynD | Codon | ATGCAATCTACACCATTACTGCAAATACAACCACATTTCCATGTAG | 494 |
| | optimized | AGGTCATTGAACCAAAGCAAGTCTACTTGTTGGGTGAACAAGCTAA | |
| | | TCATGCATTGACAGGCCAATTATACTGCCAAATTTTGCCATTGTTA | |
| | | AACGGACAATACACATTGGAACAAATCGTTGAAAAACTAGACGGAG | |
| | | AAGTACCACCTGAATACATTGATTATGTGCTGGAGAGACTAGCTGA | |
| | | GAAGGGCTATCTGACTGAAGCAGCACCTGAATTATCTAGTGAAGTG | |
| | | GCCGCTTTCTGGTCTGAGCTGGGGATTGCACCTCCTGTCGCGGCCG | |
| | | AAGCATTACGTCAACCTGTGACTTTAACACCTGTTGGAAACATCAG | |
| | | CGAAGTAACAGTAGCAGCCTTAACCACAGCCCTACGTGATATCGGT | |
| | | ATTTCCGTTCAAACACCTACAGAAGCTGGATCGCCAACTGCATTGA | |
| | | ACGTTGTACTTACCGATGATTATCTCCAACCAGAACTCGCTAAGAT | |
| | | CAATAAGCAAGCCTTAGAAAGTCAACAAACTTGGCTACTTGTCAAA | |
| | | CCAGTTGGCTCCGTGTTATGGTTGGGTCCGGTATTCGTGCCAGGAA | |
| | | AAACAGGTTGCTGGGATTGTTTGGCTCACAGATTAAGGGGGAATAG | |
| | | AGAGGTAGAGGCCTCTGTATTGAGACAAAAACAAGCTCAACAACAA | |
| | | CGTAATGGACAAAGCGGGTCTGTAATAGGATGCCTTCCCACGGCTA | |
| | | GAGCGACACTGCCCTCAACACTCCAAACTGGGCTGCAGTTCGCTGC | |
| | | TACCGAAATTGCTAAATGGATAGTTAAGTATCATGTTAATGCCACA | |
| | | GCGCCTGGCACCGTATTCTTCCCTACATTGGATGGTAAGATAATTA | |
| | | CGCTAAATCACTCCATACTGGATTTGAAGTCACATATTCTGATCAA | |
| | | GCGTTCTCAATGTCCCACCTGTGGTGACCCAAAAATCTTACAGCAC | |
| | | CGTGGTTTCGAACCTTTAAAACTTGAGTCAAGGCCTAAACAGTTCA | |
| | | CCTCAGACGGCGGACATCGTGGTACTACCCCTGAACAAACTGTCCA | |
| | | GAAATATCAACATTTAATCTCGCCTGTTACCGGTGTAGTTACTGAA | |
| | | TTGGTCAGGATAACTGATCCGGCCAATCCACTAGTTCACACATATA | |
| | | GAGCTGGTCATAGCTTCGGGAGCGCTACATCGCTGAGAGGGCTGCG | |
| | | TAATACCTTAAAGCATAAGAGTTCAGGTAAGGGTAAGACTGATTCT | |
| | | CAAAGTAAAGCCTCGGGCCTGTGTGAGGCGGTAGAACGTTACTCAG | |
| | | GAATCTTTCAAGGTGACGAACCGAGAAAACGCGCCACATTGGCTGA | |
| | | ATTGGGAGATTTGGCAATTCACCCTGAGCAATGCTTGTGTTTTTCC | |
| | | GACGGTCAGTACGCTAATAGAGAAACTTTAAACGAACAGGCAACGG | |
| | | TGGCACATGATTGGATACCTCAACGTTTTGATGCATCACAAGCTAT | |
| | | TGAATGGACTCCAGTCTGGTCCCTAACTGAACAGACCCATAAATAT | |
| | | TTGCCCACCGCATTGTGTTACTACCATTATCCTCTACCCCCAGAAC | |
| | | ACAGATTCGCACGTGGAGATTCGAATGGTAATGCTGCCGGAAATAC | |
| | | GTTGGAAGAGGCTATACTCCAAGGCTTCATGGAATTAGTCGAGAGA | |
| | | GATGGTGTGGCTTTATGGTGGTATAACAGGCTACGCAGACCCGCTG | |
| | | TAGACTTAGGCTCATTTAACGAGCCATACTTCGTTCAGTTGCAACA | |
| | | ATTCTACAGAGAAAACGATAGAGATTTGTGGGTTTTGGACTTGACA | |
| | | GCTGATTTAGGTATCCCGGCTTTCGCGGGCGTTTCTAATAGAAAAA | |
| | | CTGGTAGTTCGGAGAGGTTGATATTAGGATTCGGTGCACACCTCGA | |
| | | TCCTACTATTGCAATTCTGAGAGCAGTTACAGAAGTTAACCAGATT | |
| | | GGCCTTGAATTAGATAAAGTTCCAGACGAGAACCTTAAGAGCGACG | |
| | | CAACAGATTGGCTAATTACTGAAAAATTAGCTGACCACCCTTATTT | |
| | | GTTACCAGATACAACTCAACCTCTAAAAACTGCTCAAGATTATCCT | |
| | | AAAAGGTGGTCTGACGATATATACACGGACGTAATGACTTGCGTTA | |
| | | ATATTGCTCAACAAGCAGGACTTGAAACTCTAGTTATTGATCAAAC | |
| | | ACGTCCGGACATTGGTTTGAATGTTGTTAAGGTGACAGTCCCGGGG | |
| | | ATGAGGCACTTTTGGTCAAGATTTGGAGAGGGGAGGCTTTATGACG | |
| | | TGCCCGTCAAATTAGGTTGGCTTGACGAACCTTTGACCGAAGCGCA | |
| | | AATGAACCCCACGCCGATGCCTTTT | |
| mcbCD | Synthesized | ATGTCAAAACACGAACTCTCTTTAGTGGAAGTAACGCATTACACAG | 495 |
| | without codon | ATCCTGAAGTTCTGGCCATTGTTAAAGATTTTCATGTCAGAGGTAA | |
| | optimization | CTTTGCTTCCCTCCCCGAATTTGCTGAACGAACTTTCGTGTCCGCG | |
| | as overlapping | GTACCTCTTGCCCATCTGGAGAAATTTGAAAATAAAGAAGTTCTCT | |
| | reading frames | TCAGGCCAGGTTTCAGCTCCGTAATAAACATATCCTCATCACATAA | |
| | (same as | TTTTAGTCGTGAAAGGCTCCCATCAGGAATAAACTTTTGCGACAAA | |
| | native E. coli | AATAAACTTTCCATTCGTACTATTGAAAAGTTATTAGTCAATGCAT | |
| | cluster) | TCAGCTCACCTGATCCTGGCTCTGTAAGGCGGCCTTATCCTTCTGG | |
| | | GGGGGCATTGTACCCGATTGAAGTTTTTTTATGCAGATTATCTGAA | |
| | | AATACAGAAAACTGGCAGGCAGGAACTAATGTTTATCACTACCTGC | |
| | | CGCTAAGTCAGGCACTAGAACCTGTTGCTACATGTAATACTCAGTC | |
| | | ACTCTACCGAAGCCTGTCCGGTGGGGATTCGGAACGTCTTGGTAAA | |
| | | CCCCATTTTGCTCTCGTCTATTGCATTATTTTTGAAAAAGCTTTGT | |
| | | TCAAATATCGCTACAGAGGATACCGGATGGCCTTAATGGAAACAGG | |
| | | TTCGATGTATCAGAACGCAGTATTGGTTGCAGATCAAATAGGACTG | |
| | | AAAAACCGGGTATGGGCGGGATATACCGATTCATACGTAGCAAAAA | |
| | | CAATGAATCTGGATCAGAGGACTGTAGCGCCACTGATCGTTCAGTT | |
| | | TTTTGGAGATGTAAACGATGATAAATGTCTACAGTAACCTTATGTC | |
| | | CGCATGGCCGGCCACAATGGCCATGAGTCCAAAACTGAACAGAAAT | |
| | | ATGCCAACGTTTTCTCAGATATGGGACTATGAGCGTATTACACCAG | |
| | | CCAGCGCGGCCGGTGAAACTCTGAAGTCAATTCAGGGGGCAATAGG | |
| | | TGAATATTTTGAACGCCGTCATTTTTTTAATGAGATAGTCACCGGT | |
| | | GGTCAGAAAACATTATATGAGATGATGCCTCCATCTGCTGCAAAGG | |
| | | CTTTTACCGAAGCATTTTTTCAGATCTCATCACTGACCCGCGATGA | |
| | | AATCATAACCCATAAATTTAAAACGGTCAGAGCCTTTAATCTGTTT | |
| | | AGCCTTGAACAACAAGAAATACCTGCAGTCATAATTGCACTCGACA | |
| | | ATATAACCGCTGCAGATGATCTGAAATTTTATCCTGACAGAGATAC | |
| | | ATGCGGATGTAGCTTTCATGGTAGTTTGAACGATGCCATAGAAGGT | |
| | | TCCTTGTGTGAATTTATGGAGAGACAGTCCCTCCTTCTTTACTGGT | |
| | | TACAGGGAAAAGCCAATACTGAAATATCCAGTGAAATAGTAACAGG | |
| | | CATAAATCATATAGATGAGATTTTACTGGCTCTCAGGTCAGAAGGA | |
| | | GATATCAGGATTTTCGATATCACCCTGCCCGGAGCTCCTGGACACG | |
| | | CAGTACTAACCCTGTATGGCACAAAAAACAAAATCAGTCGAATAAA | |
| | | ATACAGTACCGGATTATCCTATGCTAATAGTCTGAAAAAAGCACTT | |
| | | TGTAAATCCGTAGTGGAATTGTGGCAATCGTATATATGCCTGCACA | |
| | | ACTTTCTTATTGGCGGTTATACTGATGATGACATTATTGATAGTTA | |
| | | CCAGCGTCACTTTATGTCATGCAACAAGTACGAGTCGTTTACGGAT | |
| | | TTGTGTGAAAATACGGTACTACTGTCTGATGATGTCAAGTTAACGT | |
| | | TTGAGGAAAATATTACGTCAGACACAAATTTATTAAACTATCTTCA | |
| | | ACAAATTTCTGATAATATTTTTGTTTACTATGCCAGGGAAAGAGTA | |
| | | AGTAACAGCCTTGTCTGGTACACAAAAATAGTAAGCCCTGATTTTT | |
| | | TCCTTCATATGAATAACTCAGGTGCAATAAACATTAATAATAAAAT | |
| | | TTACCATACCGGGGACGGTATTAAAGTCAGAGAATCAAAGATGGTA | |
| | | CCATTCCCA | |
| mdnC | Amplified | ATGACCGTTTTAATTGTTACTTTTAGCCACGATAATGAAAGTATTC | 496 |
| | from | CTCTGGTAATCAAAGCCATAGAAGCCATGGGTAAAAAAGCCTTCCG | |
| | pARW071 | TTTTGATACTGATCGCTTCCCTACAGAGGTGAAAGTTGATCTTTAC | |
| | | TCAGGCGGTCAAAAAGGCGGAATTATTACCGATGGAGAACAAAAAT | |
| | | TAGAGCTAAAAGAAGTTTCTTCTGTCTGGTATCGACGCATGAGATA | |
| | | CGGACTAAAATTACCCGATGGGATGGATAGTCAATTTCGCGAAGCT | |
| | | TCTCTTAAGGAATGTCGGTTAAGTATTCGAGGAATGATTGCTAGTT | |
| | | TATCTGGCTTTCATCTTGATCCAATTGCTAAGGTAGATCATGCTAA | |
| | | TCATAAACAATTGCAGTTACAAGTGGCGCAACAATTAGGTTTATTA | |
| | | ATTCCGGGGACTTTAACTTCTAATAATCCTGAAGCTGTCAAGCAAT | |
| | | TTGCTCGGGAGTTTGAAGCGACGGGAATTGTGACTAAAATGCTTTC | |
| | | TCAATTTGCTATTTATGGAGACAAGCAAGAGGAAATGGTTGTTTTT | |
| | | ACCAGTCCTGTTACAAAGGAAGATCTAGATAATTTGGAAGGTTTGC | |
| | | AATTTTGTCCAATGACTTTTCAGGAAAACATTCCTAAAGCTTTGGA | |
| | | ATTACGCATCACTATCGTCGGTGAACAAATATTTACGGCGGCGATT | |
| | | AATTCCCAACAATTAGACGGTGCTATCTACGATTGGCGAAAAGAGG | |
| | | GACGCGCGCTCCATCAACAATGGCAACCCTACGATTTACCGAAAAC | |
| | | TATTGAAAAACAACTACTAGAATTAGTGAAATATTTCGGTCTTAAT | |
| | | TATGGTGCAATTGATATGATTGTCACACCAGATGAACGTTATATCT | |
| | | TTTTAGAAATTAATCCCGTTGGCGAGTTTTTCTGGCTAGAACTTTA | |
| | | TCCTCCTTATTTTCCTATCTCCCAGGCGATCGCTGAAATCCTAGTT | |
| | | AACTCA | |
| mibD | Codon | ATGACGGCACACAGCGACGCAGGAGGTGACCCACGCCCGCCTGAAC | 497 |
| | optimized | GCTTACTGTTGGGGGTGTCAGGAAGTGTCGCTGCACTGAACTTACC | |
| | | GGCGTACATTTATGCCTTTCGGGCAGCCGGTGTGGCACGTCTTGCG | |
| | | GTCGTGCTGACACCAGCGGCTGAAGGGTTCCTTCCAGCGGGTGCGT | |
| | | TACGCCCGATTGTGGATGCCGTTCATACGGAACATGACCAAGGCAA | |
| | | AGGTCACGTAGCGCTGTCACGCTGGGCGCAACACTTACTCGTGCTG | |
| | | CCGGCAACAGCGAATTTGCTTGGCTGTGCAGCGTCAGGACTTGCGC | |
| | | CGAACTTTTTAGCGACCGTTCTGCTCGCGGCAGATTGCCCAATCAC | |
| | | ATTCGTCCCGGCGATGAATCCGGTCATGTGGCGTAAACCAGCCGTA | |
| | | CGCCGGAACGTTGCAACCTTACGCGCAGATGGTCATCACGTGGTGG | |
| | | ATCCTCTGCCGGGCGCTGTGTACGAAGCTGCCTCACGTTCTATCGT | |
| | | GGAAGGTCTTGCTATGCCGCGCCCTGAAGCGTTAGTCCGTTTACTG | |
| | | GGTGGCGGTGATGACGGTTCTCCAGCAGGACCGGCAGGTCCGGTTG | |
| | | GACGCGCAGAGCATGTTGGGGCTGTTGAGGCTGTTGAAGCCGTGGA | |
| | | AGCAGTTGAGGCCGTTGAGGCTGCGGAAGCACTTGCG | |
| mibH | Codon | ATGGCACGTAGTGAGGAATCGAACACTCTGGCACGTCTGTTTGACG | 498 |
| | optimized | TGTTGGGTGACGATGCCGCTGCCGCACGTGAATGGGTAACGGAACC | |
| | | CCATCGTCTGATCGCTAGCAATGAGCGCCTGGGCACAGCTCCGGAA | |
| | | GCCCCGGCGGATGACGATCCGGAGGCCATTCGGACGGTTGGAGTGA | |
| | | TCGGAGGGGGCACAGCCGGGTATTTAACGGCGTTGGCTCTGAAGGC | |
| | | TAAACGCCCTTGGTTGGATGTGGCGCTCGTCGAAAGTGCGGATATC | |
| | | CCGATCATTGGGGTAGGAGAGGCGACGGTGTCTTATATGGTGATGT | |
| | | TTCTGCACCATTATCTGGGCATTGATCCGGCGGAGTTTTACCAACA | |
| | | TGTGCGCCCTACTTGGAAACTGGGCATCCGTTTTGAATGGGGGTCA | |
| | | CGTCCGGAGGGCTTTGTTGCGCCATTCGATTGGGGGACCGGATCTG | |
| | | TTGGCCTGGTTGGGAGCCTGCGTGAAACGGGCAATGTCAACGAAGC | |
| | | TACGTTACAGGCGATGCTCATGACGGAGGATCGCGTTCCGGTATAT | |
| | | CGTGGCGAAGGTGGGCATGTTAGTCTGATGAAATATCTGCCATTCG | |
| | | CATATCATATGGATAACGCTCGCCTGGTTCGCTACCTGACGGAACT | |
| | | CGCCACTCGTCGTGGCGTGCATCATGTCGATGCGACTGTAGCTGAA | |
| | | GTTCGCCTGGATGGTCCTGACCACGTTGGGGACCTGATTACTACGG | |
| | | ACGGTCGTCGCCTGCACTATGACTTTTACGTCGATTGTACTGGATT | |
| | | TCGTTCCCTGCTGCTGGAAAAAGCCCTGGGTATCCCGTTCGAATCT | |
| | | TATGCGTCAAGCCTGTTTACCGACGCGGCAATTACCGGTACCCTTG | |
| | | CACATGGGGGTCATCTTAAACCTTACACTACGGCAACTACCATGAA | |
| | | TGCGGGCTGGTGTTGGACGATCCCTACTCCTGAGTCCGATCACCTG | |
| | | GGGTACGTTTTCAGTAGTGCCGCGATCGATCCAGACGATGCAGCAG | |
| | | CAGAAATGGCCCGCCGTTTCCCGGGCGTTACCCGCGAAGCATTAGT | |
| | | TCGCTTTCGCTCCGGCCGTCACCGTGAAGCTTGGCGCGGCAATGTC | |
| | | ATCGCGGTAGGAAACAGCTATGCTTTCGTGGAACCTCTGGAGAGTT | |
| | | CGGGACTCCTGATGATTGCTACCGCAGTCCAGATCCTGGTGAGTTT | |
| | | GCTGCCGAGTAGTCGTCGTGACCCGCTGCCTAGCAATGTGGCGAAT | |
| | | CAGGCGTTAGCTCACCGGTGGGACGCGATTCGTTGGTTTCTGAGTA | |
| | | TTCATTACCGTTTCAACGGCCGCCTCGATACTCCGTTCTGGAAGGA | |
| | | AGCCCGTGCCGAAACAGATATTAGCGGTATTGAACCGTTGCTTCGT | |
| | | CTGTTCAGTGCCGGTGCCCCTCTGACCGGTCGCGATAGCTTTGCGC | |
| | | GCTATTTGGCCGACGGAGCAGCCCCGTTGTTCTATGGCCTGGAGGG | |
| | | TGTTGATACCTTACTGCTGGGACAGGAAGTGCCTGCGCGTCTGTTA | |
| | | CCACCGCGTGAATCTCCTGAGCAGTGGCGTGCCCGTGCTGCAGCAG | |
| | | CCCGCTCATTAGCCTCGCGTGGCTTACGTCAGAGCGAAGCTCTGGA | |
| | | TGCTTACGCTGCGGACCCCTGTCTCAATGCGGAACTGCTGTCTGAT | |
| | | AGCGACTCATGGGCGGGTGAACGCGTCGCGGTACGTGCAGGTCTGC | |
| | | GT | |
| mibO | Codon | ATGATTTTTGGCCCGGATTTTCATCGCGATCCGTATCCAGTGTATC | 499 |
| | optimized | GTCGTCTGCGTGATGAGGCTCCGTGCCACCATGAACCAGCGTTAGG | |
| | | TCTGTATGCGTTGAGCCGCTACGAGGACGTTCTGGCTGCCCTTCGT | |
| | | CAGCCCACCGTGTTCAGCTCAGCAGCGCGTGCGGTAGCCTCCAGTG | |
| | | CAGCGGGAGCAGGTCCATACCGCGGTGCCGACACCGTTAGTCCGGA | |
| | | GCGGGAAACTGCGGCTGAAGGGCCCGCCCGTAGCCTGTTGTTCCTG | |
| | | GATCCGCCAGAGCACCAGGTGCTGCGTCAGGCGGTGTCCCGTGGCT | |
| | | TTACGCCGCAGGCAGTATTGCGCCTTGAGCCGGCCGTCCGCGACAT | |
| | | TGCGGCGGGTCTTGCTGATCGTATCCCCGATCGCGGTGGTGGCGAG | |
| | | TTCGTTACCGAATTTGCGGCTCCGCTGGCAATCGCAGTGATTCTGC | |
| | | GGTTACTTGGTGTACCGGAAGCAGATCGTGCCCGCGTAAGCGAACT | |
| | | TTTATCGGCATCAGCCCTGTCGGGGGCGGAAGCAGAACTGCGCTCC | |
| | | TATTGGCTGGGCCTTTCGGCACTCCTCCGCGATCGTGAAGATGCAG | |
| | | GCGAAGGTGACGGAGAGGATCGTGGTGTGGTGGCGGCTCTGGTCCG | |
| | | TCCTGATGCTGGACTGCGCGACGCGGATGTTGCCGCAGGACCTGCC | |
| | | GTGCGTGCACCGCTGACGGATGAGCAGGTTGCAGCATTCTGCGCCT | |
| | | TAGTGGGGCAAGCCGGCACTGAAAGTGTGGCAATGGCGCTCTCCAA | |
| | | CGCATTGGTCCTGTTCGGGCGTCACCATGACCAGTGGCGCACACTG | |
| | | TGTGCGCGTCCGGATGCGATTCCAGCAGCATTCGAAGAGGTCCTCC | |
| | | GCTATTGGGCACCTACGCAGCATCAAGGTCGGACGTTAACCGCGGC | |
| | | GGTACGTTTACATGGCCGTCTGCTGCCGGCCGGTGCGCATGTACTG | |
| | | CTGCTGACCGGTTCAGCCGGCCGGGATGAACGTGCGTACCCAGACC | |
| | | CCGATGTATTTGACATCGGTCGCTTCCACCCGGATCGTCGTCCGTC | |
| | | GACCGCGCTGGGTTTTGGTCTGGGCGCACACTTTTGTTTAGGCGCT | |
| | | GCTCTCGCTCGTCTGCAGGCACGCGTAGCGCTGCGCGAACTGACAC | |
| | | GCCGGTTCCCGCGTTATCGTACGGACGAGGAACGCACTGTGCGTTC | |
| | | GGAAGTGATGAACGGGTTCGGCCACAGCCGTGTACCATTTTCCACG | |
| mibS | Codon | ATGACGACTGGCACCACGGTAGCGCATGCTGTAGAACCAGACGGTT | 500 |
| | optimized | TCCGCGCCGTGATGGCCACACTGCCGGCCGCTGTGGCGATCGTTAC | |
| | | GGCAGCTGCGGCAGATGGGCGCCCGTGGGGTATGACCTGCAGTTCG | |
| | | GTTTGCTCAGTGACCTTGACCCCGCCGACCCTTCTGGTCTGCCTTC | |
| | | GGACGGCGTCCCCGACTCTGGCCGCAGTCGTGTCAGGTCGTGCATT | |
| | | TAGCGTGAACCTTCTGTGTGCGCGGGCCTATCCTGTGGCGGAATTG | |
| | | TTTGCATCTGCGGCAGCAGACCGGTTTGATCGCGTTCGTTGGCGTC | |
| | | GCCCGCCGGGTACAGGCGGTCCACATCTTGCCGATGATGCACGTGC | |
| | | AGTGTTAGACTGTCGCCTGAGCGAAAGCGCAGAAGTAGGCGACCAT | |
| | | GTGGTCGTATTTGGCCAAGTCCGGGCGATTCGTCGCCTGAGTGATG | |
| | | AACCACCACTGATGTATGGTTATCGTCGTTACGCACCTTGGCCGGC | |
| | | AGATCGTGGTCCGGGTGCGGCAGGCGGC | |
| paaA | Codon | ATGAGCCTGACGAATGTCAAGCCGTTGATTAAAGAATCCCACCACA | 501 |
| | optimized | TCATTTTAGCTGACGATGGTGACATTTGCATTGGGGAAATTCCGGG | |
| | | GGTGTCTCAGGTAATCAATGACCCGCCGTCGTGGGTTCGTCCTGCC | |
| | | CTGGCAAAGATGGATGGCAAGCGTACTGTCCCCCGTATTTTCAAAG | |
| | | AACTGGTCAGTGAAGGCGTACAGATCGAATCCGAACATCTGGAAGG | |
| | | CCTGGTAGCCGGGCTTGCCGAACGCAAACTTCTCCAGGATAACAGT | |
| | | TTCTTTTCCAAGGTGTTAAGCGGTGAAGAAGTGGAGCGCTATAACC | |
| | | GCCAGATTCTGCAGTTCAGCCTTATCGATGCGGATAACCAGCACCC | |
| | | TTTCGTTTACCAAGAGCGGCTGAAACAGTCTAAAGTCGCTATCTTC | |
| | | GGTATGGGTGGCTGGGGCACGTGGTGTGCATTGCAGCTGGCCATGT | |
| | | CAGGCATTGGTACACTGCGGCTGATCGACGGCGATGATGTGGAACT | |
| | | GTCGAACATTAACCGCCAAGTTCTGTATCGCACGGATGATGTAGGT | |
| | | AAAAACAAAGTTGATGCCGCCAAAGACACTATCCTGGCATACAACG | |
| | | AAAACGTGCATGTTGAAACCTTCTTTGAATTCGCCAGCCCGGACCG | |
| | | TGCCCGGCTTGAAGAACTTGTGGGTGATTCTACCTTTATTATCCTG | |
| | | GCTTGGGCCGCGTTGGGTTACTACCGTAAAGATACGGCAGAGGAAA | |
| | | TTATCCATTCGATTGCGAAAGATAAAGCGATCCCTGTAATTGAACT | |
| | | CGGCGGTGATCCTTTGGAAATCTCTGTCGGTCCTATTTACCTGAAT | |
| | | GATGGCGTACACAGCGGCTTCGACGAGGTGAAAAATTCCGTTAAAG | |
| | | ATAAATACTACGACAGCAACAGCGATATCCGCAAATTTCAAGAGGC | |
| | | GCGGTTGAAACACAGCTTCATCGATGGCGATCGTAAAGTGAACGCG | |
| | | TGGCAATCAGCGCCCAGCCTGAGTATTATGGCTGGTATCGTAACGG | |
| | | ATCAGGTTGTGAAAACCATTACCGGGTACGACAAGCCACATCTCGT | |
| | | TGGCAAGAAATTTATCTTGAGTCTGCAAGATTTCCGCAGCCGCGAG | |
| | | GAGGAGATCTTTAAA | |
| padeK | Codon | ATGACCGAACGTGCCGCAGTGCGTACCGACCATTATAAAGCCTTTG | 502 |
| | optimized | GGTTTAGAATTGAAAGCGATTTCGTGCTCCCGGAACTTCCGCCCGC | |
| | | AGGCGAACGCGAACCGCTCGATAATATTACGGTTCGTCGTACCGAC | |
| | | CTGCAGCCGCTCTGGAATTCTAGTATCCATTTTTACGGAAACTTTG | |
| | | CCATTCTGGATCACGGACGCACGGTTATGTTTCGAGTTCCGGGTGC | |
| | | TGCTATCTATGCGGTACAGGATGCTAGCAGCATATTAGTGTCCCCA | |
| | | TTCGATCAGGCAGAAGAAAACTGGGTACGTCTTTTTATTCTGGGTA | |
| | | CCTGTATTGGGATCATCCTGCTGCAGCGTAAGATTATGCCGCTGCA | |
| | | CGGTAGCGCCGTTGCCATTGATGGCAAAGCCTACGCGATTATCGGC | |
| | | GAATCTGGTGCCGGCAAAAGCACTCTTGCACTGCATCTTGTCAGTA | |
| | | AGGGTTATCCATTGCTTTCGGATGATGTGATTCCGGTCGTTATGAC | |
| | | CCAGGGCTCCCCCTGGGTGGTGCCGTCGTACCCGCAACAAAAACTT | |
| | | TGGGTGGACACTCTGAAGCACATGGGAATGGATAATGCAAACTATA | |
| | | CGCCGCTGTACGAACGTAAAACGAAGTTCGCGGTGCCCGTGGGCAG | |
| | | TAATTTCCACGAAGAACCGCTGCCGTTAGCTAGCATTTTCGAGCTT | |
| | | GTCCCGTGGGATGCGGCAACGCACATTGCCCCGATCCAAGGGATGG | |
| | | AACGCTTTCGTGTCCTGTTCCACCACACTTATCGGAACTTTCTGGT | |
| | | TCAGCCGCTGGGTCTTATGGAATGGCATTTTAAAACTCTGAGCTCG | |
| | | TTCGTTCACCAAATTGGAATGTATCGTCTGCATAGACCTATGGTCG | |
| | | GATTCAGTACCTTAGATTTAACGTCGCACATTCTGAATATAACGCG | |
| | | TCAGGGAGAGAACGATCAA | |
| palS | Codon | ATGGGGAATTTGCGTGATTTCTACCAACTGATGAAAGATAACTATG | 503 |
| | optimized | CGGACTCTAATCTGTTCAAGGATTTGAATCTGATCCACAATATCTC | |
| | | CAACGACATCCAAATTGGAATTAATTGCGATTTCTCTGAAATGCTG | |
| | | GGAGAACTGGTAGGTAATTACGATTCCCTGAACTATCCGTCAATCA | |
| | | CCTGTGGTATTCTGACGTATAATGAAGAACGCTGCATTAAACGTTG | |
| | | TCTGGAAAGTGTGGTGAACGAATTCGATGAGATTATTGTCTTGGAT | |
| | | AGTGTATCCGAGGACAATACCGTGAAAATTATCAAGGAGAATTTCA | |
| | | ACGATGTCAAAGTCTACGTCGAGCCATGGAAGAACGATTTTTCATT | |
| | | TCACCGCAACAAGATCATTAATCTCGCAACGTGCGACTGGATCTAC | |
| | | TTTATCGACGCGGATAATTATTATGATTCGAAGAACAAGGGTAAAG | |
| | | CCATGCGCATCGCTAAGGTTATGGATTTCTTGAAAATCGAAGGCGT | |
| | | TGTGAGCCCAACGGTCATTGAGCATGACAATAGCATGAGCCGTGAT | |
| | | ACCCGTAAGATGTTTCGTCTGAAAGATAACATTCTGTTTAGCGGTA | |
| | | AAGTTCATGAAGAACCGGTGTATGCCAATGGTGAGATCCCCCGGAA | |
| | | CATCATAGTAGACATCAACGTGTTTCACGACGGCTATAACCCAAAG | |
| | | ATTATCAACATGATGGAAAAGAACGAGCGCAATATCACCCTGACTA | |
| | | AAGAGATGATGAAGATCGAACCGAACAATCCGAAATGGCTGTACTT | |
| | | CTATAGCCGCGAACTCTATCAGACGCAACGTGACATTGCCCTTGTG | |
| | | CAAAGTGTACTGTTCAAGGCACTGGAACTGTATGAAAACAGTTCAT | |
| | | ATACGCGTTATTATGTTGACACCATTGCCTTACTGTGCCGAGTGCT | |
| | | GTTCGAATCTAAAAACTACCAGAAACTTACGGAATGTCTGAACATC | |
| | | CTGGAGAACAATACGCTTAACTGTTCCGATATCGATTACTATAATT | |
| | | CAGCGCTGCTGTTCTACAACCTGTTACTGCGCATCAAGAAAATTAG | |
| | | CTCCACCCTGAAGGAGAACATTGATATGTACGAACGTGACTATCAT | |
| | | AGCTTTATCAACCCCTCGCATGATCACATTAAGATTCTGATATTAA | |
| | | ATATGCTCCTGCTGCTCGGCGATTACCAGGATGCCTTTAAGGTTTA | |
| | | CAAGGAGATCAAGTCCATTGAGATTAAAGATGAGTTTCTGGTGAAC | |
| | | GTGAACAAATTCAAAGACAATCTTCTGAGCTTCATTGACTCCATTA | |
| | | ACAAAATT | |
| papB | Codon | ATGGCAAACCTGATCCAGGACCGCGAGGACGAACTGATTCATTTCC | 504 |
| | optimized | ATCCGTACAAACTGTTCGAGGTGGATTCAAAAACCTTCTTCTATAA | |
| | | CGTAGTCACCAACGCGATTTTTGAAATTGATAGCCTGATAATCGAC | |
| | | ATTCTTCACTCAAAAGGTAAAAATGAGGAGCACGTTGTGAAAGATT | |
| | | TGGCTGAACGCTATGAGCTGTCTCAGGTTCGCGAAGCGATCCAGAA | |
| | | CATGAAAGAGGCATACATTATAGCAACCGATGCTAACATCTCCGAC | |
| | | GTAGAGAAGATGGGTATCTTAGATAACTCGCAGCGCGTTTTTAAAC | |
| | | TGTCTAGCCTGACGCTCTTTATGGTGCAGGAATGCAACCTGCGGTG | |
| | | TACGTATTGTTACGGCGAAGAAGGAGAATACAACCAGAAAGGTAAA | |
| | | ATGACGTCCGAAATCGCCCGGAGCGCAGTGGATTTTCTGATTCAAC | |
| | | AGAGTGGTGAAATCGAACAGTTGAACATCACATTCTTTGGAGGCGA | |
| | | ACCGCTGCTCAACTTTCCATTAATACAAGAAACCGTGCAGTATGTG | |
| | | CACGAACAGAGCGAGATCCATAACAAGAAATTTAGCTTTTCCATCA | |
| | | CCACCAATGGCACGCTCATTACCCCCAAAATCAAAAACTTCTTCTA | |
| | | TAAACACCACTTTGCAGTCCAGACTTCTATCGATGGTGATGAAAAG | |
| | | ACGCACAATTTCAATCGCTTCTTCAAAGGAGGCCAGGGCTCTTATG | |
| | | ATCTGCTGTTAAAGCGGACGGAAGAAATGCGCAATGACCGTAAAAT | |
| | | TGGTGCACGTGGAACCGTGACCCCTGCCGAGCTGGACCTCTCAAAA | |
| | | TCATTTGACCACTTAGTTAAACTCGGCTTTCGCAAAATCTACTTAT | |
| | | CACCCGCTTTATATAGTCTCTCTGACGATCACTACGACACCCTGAG | |
| | | CAAAGAGATGGTCAAACTTGTTGAACAATTCCGTGAGCTGCTGGAG | |
| | | CGTGAAGATTACGTCACCGCGAAGAAAATGTCTAATGTTCTGGGTA | |
| | | TGTTATCGAAGATTCACTCCGGTGGCCCGCGCATTCATTTTTGCGG | |
| | | TGCCGGCACTAATGCTGCCGCTGTCGATGTCCGCGGCAACCTTTTC | |
| | | CCGTGTCATCGTTTCGTGGGTGAAGATGAATGTTCAATCGGTAACC | |
| | | TGTTCGACGAGGACCCGCTGTCAAAACAGTACAACTTTATAGAGAA | |
| | | TTCTACAGTACGCAACCGTACTACGTGTTCGAAATGCTGGGCGAAG | |
| | | AATCTGTGCGGCGGTGGTTGTCACCAAGAAAATTTCGCCGAGAATG | |
| | | GTAATGTGAACCAGCCAGTGGGCAAATTATGCAAAGTGACCAAAAA | |
| | | CTTCATCAACGCGACCATCAATCTGTACTTGCAACTTACTCAAGAA | |
| | | CAACGCAGCATTCTGTTCGGC | |
| papoK | Codon | ATGCACGATCGTAGCGCGAATGTTAGCTGGACCAAATACATCGCGT | 505 |
| | optimized | TTGGTCTGCGCATTGCCAGCGAACTCAACTTACCGGAACTGATATT | |
| | | GGCGGCTCCCGAAGCCGTTGAGGATGTTGTCATACGCCAGGCAGAT | |
| | | CTCACGGCCTGGTCTGGCCAACTTGAACAGGCAAATTTTGTCATGT | |
| | | TGGACGAACGTTTCATGTTTCAGATCCCGGGGACCGCCATTTATGC | |
| | | GGTACGCGAAGGCAAAGAGATTGAAGTGAGCATCTTCTCTGGGGCC | |
| | | GACCCGGACACCGTGCGCCTTTTCGTGCTGGGGACGTGCATGGGCG | |
| | | TGCTCTTGATGCAGCGCCGCATTCTGCCTATCCACGGCTCCGCCGT | |
| | | CGTTATCGGTGGCCGCGCGTATGCCTTTGTTGGTGAATCAGGCACA | |
| | | GGTAAATCGACCTTAGCTGCAGCATTTCGGCAGGCCGGTTACCAAA | |
| | | TGGTTAGCGATGATGTCATTGCCGTCAAAGCGACCGCATCTAGCGC | |
| | | TATTGTTTACCCTGCGTATCCACAGCAAAAACTGGGTTTAGATTCG | |
| | | CTGTTGCAGCTTGAAGCGCTCCGTGAGAATAAGCACGCCCGCAAGC | |
| | | GTAACAACATCCGTTCTCTGACGGATGGCAATAGTGTGATGCCGCA | |
| | | GTACAGCGATCTGCGCATGCTGGCGGGGGAACTGAATAAATATGCA | |
| | | GTTCCAGCCGTCGATGAATTCTTTAATGACCCGCTGCCGTTGGGCG | |
| | | GTGTTTTCGAACTGGTAGCAGACAGTCCGATTCGAGCATTAATGCG | |
| | | CGAAGGCGAACTCGTCGCTGTGACCGAGCAACCGCTGAACGTTCTG | |
| | | GAATGTTTACATACTCTTCTGCAACACACGTACCGTCGGGTAATCA | |
| | | TCCCTCGAATGGGACTGAGCGAGTGGAGCTTCGATACTGCGGCCCG | |
| | | AATGGCACGCAAGGTCGAGGGCTGGCGACTCCTCCGTGATAGCTCC | |
| | | GTGTTCACGGCTAGTGAAGTCGTCCAGCGCGTCCTCGACATCATCC | |
| | | GTAAGGAGGAAAAGAGCTACGGATCACAC | |
| pbtM1 | Codon | ATGCTGTCTAGCGCGCTGGAGGTGGATATCGATGAAGCTGCGGTGG | 506 |
| | optimized | CGGCGGACTTACGCGAATTGGCCGCAGCTCTGGATCGCAGTGGTTA | |
| | | TGGTGAAATCCTCACCTGTTTTCTGCCTCAGAAGGCACAGGCGCAT | |
| | | ATCTGGGCTCAGACCGCTGCAAAAATTGATGGGCCGTTGCGTACCC | |
| | | TGATGGAATTATTCCTTCTGGGTCGGGCGGTTCCCCAGGATGATCT | |
| | | CCCGCCTCGCATCGCGGCCGTGATTCCCGGTTTAGTTAGCGCAGGT | |
| | | CTGGTTAAGACTGGACAGGGCGCGGTTTGGCTGCCGAACTTGATTC | |
| | | TGCTGCGTCCTATGGGCCAGTGGTTATGGTGTCAGCGGCCTCACCC | |
| | | CTCACCGACCATGTACTTTGGTGACGATAGCCTGGCGCTGGTTCAC | |
| | | CGGATGGTAACATATCGTGGCGGCCGTGCCCTGGATTTATGTGCAG | |
| | | GTCCGGGTGTTCAGGCCCTTACCGCAGCCCTCCGCTCAGAGCACGT | |
| | | TACCGCGGTTGAGATCAATCCGGTCGCGGCAGCCCTTTGCCGCACC | |
| | | AACATTGCCATGAACGGTCTGTCCGACCGCATGGAGGTTCGCCTGG | |
| | | GCTCACTGTACGACGTCGTGCGCGGTGAGGTTTTTGATGATATTGT | |
| | | ATCAAACCCGCCGCTGCTGCCTGTTCCGGAGGATGTGCAATTCGCC | |
| | | TTTGTGGGAGATGGCGGACGCGATGGTTTCGATATTTCTTGGACGA | |
| | | TTCTGGATGGCCTGCCTGAACATCTGTCCGACCGTGGTGCGTGTCG | |
| | | CATCGTTGGTTGTGTTCTGTCCGATGGCTATGTGCCTGTTGTGATG | |
| | | GAAGGCTTGGGAGAATGGGCCGCTAAACACGATTTCGACGTGCTTC | |
| | | TTACAGTGACCGCACATGTCGAGGCGCATAAAGATAGTAGTTTTCT | |
| | | GCGTTCAATGAGCCTGATGAGTTCGGCGATCTCAGGCCGCCCAGCG | |
| | | GAGGAGCTGCAAGAACGGTACGCAGCTGATTATGCCGAACTGGGCG | |
| | | GTTCCCACGTTGCGTTCTATGAACTGTGTGCCCGCCGTGGTGGGGG | |
| | | TTCTGCACGTCTGGCCGACGTGAGCGCTACAAAACGCAGTGCGGAA | |
| | | GTGTGGTTTGTT | |
| pbtO | Codon | ATGACCCAGTATCCCCTGTCGCGTCCAGAACCGCTGGGCGTGCACC | 507 |
| | optimized | CAGATTATCGTCGCCTGCGTGAGACTTGCCCGGTTGCACGTGTGGG | |
| | | TAGCCCGTATGGCCCAGCGTGGCTTGTCACCCGTTACGCCGATGTG | |
| | | GCCGCAGTTCTGACCGATGCCCGTTTTAGTCGTGCAGCCGCTCCGG | |
| | | AAGATGATGGTGGCATCCTGCTGAACACCGATCCGCCGGAACATGA | |
| | | TCGTCTGCGTAAACTGATTGTAGCACACACAGGCACCGCTCGCGTG | |
| | | GAACGGCTGCGTCCGCGTGCTGAAGAGATCGCTGTTGCGTTAGCGC | |
| | | GCCGTATCCCGGGCGAAGGCGAATTCATTAGTGCATTTGCCGAGCC | |
| | | CTTCAGCCATCGCGTTTTGTCTTTATTTGTTGGCCATCTTGTTGGG | |
| | | TTACCAGCGCAGGACCTGGGCCCCTTAGCGACCGTAGTGACTCTGG | |
| | | CACCCGTTCCCGACCGCGAACGTGGCGCGGCATTTGCAGAGCTGTG | |
| | | TCGTCGGCTGGGTCGTCAGGTGGATCGCGAAACGCTTGCAGTAGTT | |
| | | TTAAACGTGGTCTTTGGCGGACATGCGGCTGTAGTGGCCGCGCTGG | |
| | | GTTATTGCCTGTTAGCTGCATTAGATGCGCCACTGCCACGTCTGGC | |
| | | CGGTGACCCAGAGGGCATTGCCGAACTGGTGGAAGAAACCCTTCGT | |
| | | TTGGCTCCACCGGGAGATCGTACACTGTTGCGTCGTACTACAGAAC | |
| | | CTGTGGAACTTGGCGGTCGCACATTACCAGCGGGTGCGCTTGTAAT | |
| | | CCCGTCCATTGCAGCCGCAAACCGTGATCCGGATCGCCCTGTGGGC | |
| | | CGTCGTATGCCACGTCATCTTGCATTTGGACGTGGAGCGCATGCCT | |
| | | GTTTAGGCATGGCGCTGGCGCGCATGGAACTCCAGGCAGCACTGAA | |
| | | AGCGTTAGCGGAACACGCGCCAGACGTACGGTTGCCGGCTGGTACA | |
| | | GGCGCGCTGGTCCGCACACACGAAGAACTCTCGGTGAGCCCGCTCG | |
| | | CAGGAATCCCAATTCAACGC | |
| pcpX | Codon | ATGACATACCGTCGCACCTCCTATGCGGTATGGGAGATCACGCTGA | 508 |
| | optimized | AATGCAATCTGGCATGTTCGCACTGTGGAAGTCGTGCCGGGCACAC | |
| | | GCGAGCAAAAGAACTGTCCACACAGGAAGCGCTGGATCTGGTCCGT | |
| | | CAGATGGCTGATGTCGGCATTATCGAAGTTACTCTGATTGGGGGTG | |
| | | AAGCGTTCCTGCGTCCAGACTGGCTGCAGATTGCCGAGGCGATAAC | |
| | | GAAAGCCGGGATGCTGTGCAGCATGACTACGGGCGGTTATGGCATA | |
| | | TCGCTGGAAACCGCCCGCAAAATGAAAGCGGCAGGAATCGCGAGCG | |
| | | TGAGCGTTAGCATCGATGGCTTGGAGGAAACCCATGATCGCTTACG | |
| | | CGGTCGCAAAGGCTCTTGGCAGGCTGCGTTTAAAACAATGAGCCAT | |
| | | TTGAGAGAAGTGGGCATCTTCTTTGGCTGTAACACCCAGATTAACC | |
| | | GTCTGTCGGCCCCTGAATTTCCGCTGATATATGAACGCATCCGTGA | |
| | | CGCCGGGGCACGTGCCTGGCAGATCCAGCTTACGGTGCCGATGGGC | |
| | | CGCGCTGCCGATAACGCAAATATCCTTCTGCAACCGTACGAACTGC | |
| | | TTGATCTGTATCCGATGATTGCTCGAGTGGCCCGCCGGGCCCGTCA | |
| | | AGAGGGCGTGCAAATCCAGCCAGGTAATAATATTGGGTATTACGGC | |
| | | CCTTACGAACGTCTTTTACGTGGCCGGGGGAGCGATAGTGAGTGGG | |
| | | CATTTTGGCAGGGCTGTGCCGCGGGCTTAAGTACCCTGGGTATTGA | |
| | | AGCGGATGGTGCTATAAAAGGTTGTCCCTCACTGCCAACGAGCGCG | |
| | | TATACCGGCGGTAACATTCGCGAACATAGTCTGCGAGAAATAGTGG | |
| | | AAGAATCGGAACAGCTGCGTTTTAACCTCGGTGCAGGGACGAGCCA | |
| | | AGGGACCGCCCACTTGTGGGGCTTTTGCCAGACGTGTGAATTTAGT | |
| | | GAATTGTGCAGAGGTGGTTGTACGTGGACAGCTCACGTGTTCTTTA | |
| | | ACCGCCGTGGGAATAACCCGTATTGTCATCATCGGGCGCTTTTCCA | |
| | | AGCGGAGCAGGGTATCAGAGAACGTGTCGTGCCAAAGGTCGAAGCT | |
| | | CAGGGCCTGCCGTTTGACAACGGTGAATTTGAACTTATCGAAGAAC | |
| | | CTATTGACGCGCCTCTGCCCGAAAATGATCCACTGCACTTTACCAG | |
| | | CGACTTAGTGCAGTGGTCAGCGAGTTGGCAGGAAGAATCGGAATCT | |
| | | ATAGGCGCAGTGGTAGAC | |
| pcpY | Codon | ATGGTGGAAAACATTGATAATGAACGTGAGAAAAGTGCGAACGAAA | 509 |
| | optimized | TTGAACCGGAAAGCCTGCTTCTGCCGCGCCAGGCTTGGCAGTCGCA | |
| | | GATCGCCTATCTTAAAGCGATTCTGAAAGCCAAACAGGCGCTTGAC | |
| | | CGGATCGAAAAACGTTATCTGCGG | |
| plpX | Codon | ATGACCAAAAAGTATCGGCGTGTATCCTACGCAGTGTGGGAAATCA | 510 |
| | optimized | CCCTGAAATGCAATCTGGCATGCTCTCATTGTGGCAGCCGCGCCGG | |
| | | CCAAGCCCGTACGAAAGAGCTGAGTACCGAAGAAGCGTTCAACCTG | |
| | | GTCCGCCAGCTGGCCGACGTGGGCATTAAGGAAGTCACCCTGATCG | |
| | | GTGGTGAAGCCTTTATGCGTTCGGATTGGCTGGAAATCGCGAAAGC | |
| | | CGTCACTGAAGCCGGCATGATCTGTGGCATGACCACAGGGGGCTTC | |
| | | GGGGTCAGTCTGGAAACGGCGCGTAAAATGAAAGAAGCGGGCATTA | |
| | | AAACGGTGAGCGTTAGCATTGACGGTGGTATTCCTGAAACCCACGA | |
| | | CCGCCAGCGCGGTAAAAAGGGTGCGTGGCATAGTGCATTCCGGACT | |
| | | ATGAGCCATCTGAAAGAAGTCGGGATCTACTTCGGTTGCAACACTC | |
| | | AAATCAATCGTTTATCGGCGTCAGAATTCCCGATTATCTATGAACG | |
| | | TATTCGCGATGCTGGGGCACGTGCGTGGCAAATTCAGCTGACGGTT | |
| | | CCGATGGGCAACGCCGCGGATAACGCAGATATGCTGCTGCAACCGT | |
| | | ATGAATTGCTCGACATCTATCCGATGTTAGCCCGCGTTGCCAAACG | |
| | | TGCGAAACAGGAAGGCGTGCGTATTCAGGCAGGTAACAACATCGGG | |
| | | TACTATGGACCGTATGAGCGTCTGCTGCGTGGCAGCGACGAATGGA | |
| | | CGTTTTGGCAAGGATGTGGTGCGGGCCTTAACACCCTCGGCATCGA | |
| | | AGCCGACGGCAAAATCAAAGGCTGTCCATCCCTGCCGACCGCCGCG | |
| | | TACACCGGCGGTAACATTCGCGATCGCCCGCTGCGGGAAATCGTCG | |
| | | AACAGACCGAAGAACTGAAATTTAACTTAAAAGCTGGTACAGAACA | |
| | | AGGTACGGACCATATGTGGGGCTTTTGTAAAACCTGCGAATTCGCG | |
| | | GAACTCTGTCGCGGCGGATGCAGCTGGACTGCGCATGTGTTCTTTG | |
| | | ACCGGCGCGGCAATAATCCGTACTGCCACCATCGGGCTCTGAAACA | |
| | | AGCCCAAAAAGACATTCGCGAACGCTTTTATTTAAAAGTGAAAGCA | |
| | | AAGGGCAACCCGTTCGACAATGGTGAATTTGTTATCATTGAAGAAC | |
| | | CTTTTAACGCTCCGTTACCCGAGAATGACCTGCTGCACTTTAACAG | |
| | | TGATCACATTCAATGGCCAGAAAACTGGCAAAATAGTGAAAGCGCG | |
| | | TACGCATTGGCCAAG | |
| plpY | Codon | ATGAACAGTAATCAGATCCCTAACAAAGTTGCAACCGCGGCACAGA | 511 |
| | optimized | AATCTGACGACAGCAGCAGCGTATTACCGCGCCAGGGGTGGCAAGA | |
| | | CAAACAAGCCTTTATTAAGGCACTCATTAAAGCCAAACAGTCTCTC | |
| | | GAAATTGCCGAAATTAGCAACTTTTTAACC | |
| procM | Codon | ATGGAGAGTCCTAGCTCATGGAAAACATCGTGGCTGGCCGCCATCG | 512 |
| | optimized | CTCCGGATGAACCCCACAAATTCGACCGCCGCTTAGAATGGGACGA | |
| | | GCTTTCAGAGGAGAACTTCTTCGCAGCACTGAACTCAGAACCTGCA | |
| | | TCGTTGGAAGAGGATGATCCATGTTTTGAAGAAGCACTGCAAGACG | |
| | | CCCTGGAGGCCTTGAAGGCAGCATGGGATTTACCCCTTCTTCCCGT | |
| | | CGATAATAATCTTAATCGTCCCTTCGTAGATGTCTGGTGGCCCATT | |
| | | CGCTGTCACTCTGCGGAGAGCTTGCGTCAAAGCTTCGTCAGTGATA | |
| | | GTGCTGGACTTGCGGACGAGATTTTTGATCAGCTGGCCGATTCGTT | |
| | | ACTGGACCGTCTGTGCGCCCTGGGAGATCAGGTGTTGTGGGAGGCG | |
| | | TTTAACAAGGAGCGTACACCAGGAACGATGTTGTTAGCCCACTTAG | |
| | | GAGCCGCAGGCGACGGCTCCGGACCCCCTGTACGTGAGCATTACGA | |
| | | ACGTTTTATTCAGTCTCACCGCCGTAATGGATTAGCGCCTTTGCTT | |
| | | AAGGAATTCCCTGTACTGGGCCGCCTTATTGGAACAGTTTTGTCCC | |
| | | TTTGGTTCCAAGGGAGCGTGGAAATGCTGCAACGTATCTGCGCTGA | |
| | | CCGCACCGTTCTGCAACAGTGTTTCGCTATCCCTTGCGGGCATCAC | |
| | | CTGAAAACTGTAAAGCAGGGACTTTCTGATCCACACCGCGGCGGTC | |
| | | GCGCTGTGGCAGTTTTGGAATTTGCGGACCCAAATTCCACCGCTAA | |
| | | TTCAAGTATGCACGTAGTGTATAAACCGAAGGATATGGCTGTGGAT | |
| | | GCAGCTTACCAGGCCACCTTAGCAGATCTTAATACTCATAGCGACC | |
| | | TTTCCCCGTTGCGCACGCTTGCCATTCATAACGGCAACGGATATGG | |
| | | TTACATGGAACATGTGGTTCACCATCTTTGCGCTAACGACAAAGAG | |
| | | CTGACAAATTTCTATTTCAACGCTGGGCGTTTAACCGCGCTTCTGC | |
| | | ATCTTCTTGGATGTACTGACTGTCACCATGAAAATTTGATTGCATG | |
| | | TGGTGATCAATTACTGTTGATCGATACAGAAACATTATTGGAGGCG | |
| | | GATTTACCCGATCACATTTCGGATGCTTCGAGCACCACGGCGCAAC | |
| | | CAAAGCCTAGTAGCCTTCAAAAGCAATTTCAGCGTTCTGTTTTGCG | |
| | | TAGCGGGTTACTTCCTCAATGGATGTTCCTGGGGGAGTCGAAGTTG | |
| | | GCCATCGACATCTCGGCTCTGGGAATGTCCCCACCCAATAAGCCTG | |
| | | AGCGTATTGCACTTGGCTGGTTAGGATTCAATTCTGACGGGATGAT | |
| | | GCCTGGGCGTGTATCCCAACCAGTTGAGATTCCTACATCCTTGCCC | |
| | | GTTGGGATTGGTGAGGTTAATCCCTTTGATCGTTTTTTAGAGGATT | |
| | | TTTGTGATGGCTTTTCCATGCAATCAGAGGCCCTTATTAAGCTTCG | |
| | | CAACCGTTGGCTGGACGTTAATGGGGTTCTTGCTCATTTCGCGGGT | |
| | | CTGCCCCGCCGTATCGTTCTTCGCGCGACTCGCGTATACTTCACTA | |
| | | TCCAGCGTCAGCAGTTAGAGCCTACGGCACTGCGCTCTCCACTTGC | |
| | | ACAGGCCTTGAAACTTGAGCAGCTTACTCGTTCTTTCTTGTTGGCA | |
| | | GAGTCAAAGCCTCTTCACTGGCCCATTTTCGCAGCTGAAGTAAAGC | |
| | | AGATGCAGCACCTTGACATTCCTTTCTTCACACACTTAATCGACGC | |
| | | TGACGCTCTGCAGCTGGGCGGCCTGGAACAAGAATTACCAGGCTTC | |
| | | ATCCAGACTAGTGGCTTGGCAGCTGCTTACGAGCGTTTGCGTAATT | |
| | | TAGATACGGACGAGATTGCTTTCCAACTTCGTCTGATCCGCGGTGC | |
| | | AGTAGAGGCTCGCGAGTTGCATACTACGCCGGAGTCGAGCCCGACG | |
| | | TTGCCGCCGCCTGCCACCCCCGAGGCTCTTATGTCCTCTTCAGCCG | |
| | | AGACTAGTTTAGAAGCTGCTAAGCGCATCGCTCACCGCTTACTGGA | |
| | | GTTGGCAATTCGTGATTCTCAAGGGCAAGTAGAATGGCTGGGCATG | |
| | | GATCTGGGGGCAGATGGAGAGAGCTTCTCCTTTGGCCCAGTTGGCT | |
| | | TGAGCCTTTATGGGGGCTCAATCGGTATCGCTCACCTTCTGCAACG | |
| | | TTTGCAGGCGCAGCAAGTTTCCTTGATGGACGCAGACGCTATCCAA | |
| | | ACGGCAATTTTACAGCCCCTTGTGGGACTGGTTGATCAACCTAGCG | |
| | | ACGACGGACGTCGCCGTTGGTGGCGTGATCAGCCGCTGGGCTTAAG | |
| | | TGGATGTGGCGGTACCTTGCTTGCACTTACACTTCAAGGTGAACAA | |
| | | GCGATGGCTAATTCCCTGCTGGCCGCTGCTTTGCCCCGTTTTATCG | |
| | | AGGCTGATCAGCAACTTGACCTGATTGGTGGCTGCGCTGGACTGAT | |
| | | CGGTTCGTTGGTACAATTAGGTACTGAAAGTGCCTTACAATTAGCT | |
| | | TTGCGTGCGGGCGACCATCTTATTGCGCAACAGAATGAAGAGGGGG | |
| | | CGTGGTCTAGCTCGTCATCACAGCCCGGTTTGTTGGGCTTTAGTCA | |
| | | TGGTACTGCAGGTTACGCAGCAGCCTTAGCACACTTACATGCATTT | |
| | | TCCGCTGATGAGCGTTACCGCACCGCAGCCGCTGCCGCTTTAGCAT | |
| | | ACGAACGCGCACGTTTTAATAAAGATGCCGGCAACTGGCCAGACTA | |
| | | CCGCTCGATCGGACGTGACTCTGATTCAGATGAACCGTCCTTTATG | |
| | | GCTTCCTGGTGTCACGGCGCACCCGGCATTGCCCTGGGCCGCGCCT | |
| | | GTTTGTGGGGTACGGCGCTTTGGGACGAAGAATGCACCAAGGAGAT | |
| | | CGGAATTGGGTTACAGACCACAGCTGCTGTTTCGTCTGTTAGTACT | |
| | | GACCACCTGTGTTGTGGTTCACTTGGCCTTATGGTATTATTAGAGA | |
| | | TGCTGTCAGCAGGACCCTGGCCCATCGACAATCAATTACGTTCCCA | |
| | | TTGCCAGGACGTAGCATTCCAGTACCGCCTGCAGGCTTTGCAGCGC | |
| | | TGTTCAGCCGAGCCGATTAAGCTTCGTTGCTTCGGTACAAAAGAGG | |
| | | GCCTTTTAGTCCTGCCTGGATTTTTCACTGGCTTATCAGGAATGGG | |
| | | TTTAGCACTGCTTGAGGATGATCCATCTCGCGCCGTGGTTTCTCAA | |
| | | CTGATCAGTGCGGGCTTATGGCCGACAGAG | |
| psnB | Codon | ATGACGAATTTAGACACGAGCATTGTGGTCGTAGGAAGTCCGGATG | 513 |
| | optimized | ATCTTCACGTCCAGTCAGTGACGGAGGGTCTGCGTGCACGCGGTCA | |
| | | CGAGCCTTACGTGTTTGACACCCAACGTTTTCCGGAAGAGATGACA | |
| | | GTGTCACTTGGTGAACAGGGTGCCTCTATTTTTGTCGATGGCCAGC | |
| | | AAATTGCACGTCCGGCGGCGGTGTACCTCCGTTCACTGTACCAGAG | |
| | | CCCCGGCGCGTATGGGGTGGATGCCGACAAAGCGATGCAGGATAAC | |
| | | TGGCGCCGCACATTGCTCGCTTTTCGCGAGCGTAGTACCCTGATGA | |
| | | GCGCTGTGCTTCTGCGTTGGGAAGAAGCGGGGACTGCAGTGTATAA | |
| | | TTCGCCACGCGCGTCGGCGAATATCACTAAACCGTTTCAGCTGGCG | |
| | | CTGCTGCGCGACGCTGGTCTGCCGGTACCACGTAGCTTGTGGACAA | |
| | | ACGACCCTGAAGCAGTGCGGCGGTTTCATGCGGAAGTGGGTGACTG | |
| | | TATTTACAAACCGGTCGCCGGGGGAGCGCGTACACGCAAACTGGAA | |
| | | GCGAAAGATCTCGAAGCGGACCGCATCGAACGCCTGAGTGCAGCGC | |
| | | CGGTGTGTTTTCAAGAACTGCTCACAGGAGATGATGTGCGTGTTTA | |
| | | CGTGATAGATGACCAGGTAATATGCGCCCTGCGCATCGTAACTGAT | |
| | | GAGATCGATTTCCGCCAAGCAGAGGAACGTATCGAGGCCATCGAAA | |
| | | TTTCAGATGAAGTAAAAGACCAATGTGTACGTGCCGCCAAACTTGT | |
| | | TGGCCTGCGCTACACCGGTATGGATATCAAAGCCGGCGCCGATGGT | |
| | | AACTATCGTGTTCTCGAACTGAACGCGAGTGCGATGTTTCGCGGTT | |
| | | TCGAAGGCCGTGCGAATGTGGATATCTGTGGACCGCTGTGTGATGC | |
| | | ATTGATCGCTCAGACCAAACGT | |
| raxST | Codon | ATGGATTATCATTTCATCAGCGGACTGCCTCGTGCGGGGAGTTCAT | 514 |
| | optimized. ST | TACTGGCTGCGTTACTGCGTCAAAATCCGCAGCTGCATGCCGATGT | |
| | stands for | TACATCTCCGGTGGCGCGCCTTTACGCGGCCATGCTGATGGGTATG | |
| | SulfoTransferase | AGTGAAGAACACCCGAGCAACGTGCAGATTGACGATGCCCAACGTG | |
| | and denotes | TCCGTCTGTTACGTGCAGTATTTGATGCGTATTATCAGAACCGTCA | |
| | a single gene, | GGAACTGGGGACAGTGTTCGATACTAACCGCGCATGGTGCTCTCGC | |
| | not two genes. | CTCACGGGCCTGGCGCGTCTGTTTCCGCGTAGTCGCATGATCTGCT | |
| | | GTGTACGCGATGTGGGCTGGATTGTTGATTCTTTTGAACGCCTGGC | |
| | | GCAGTCGCAGCCGTTACGCCTTTCGGCCCTGTTCGGTTACGACCCC | |
| | | GAGGATTCGGTTAGCATGCACGCTGACTTACTCACTGCGCCTCGCG | |
| | | GGGTAGTGGGCTACGCCCTGGATGGTTTACGTCAAGCGTTTTATGG | |
| | | AGATCACGCGGATCGTCTGCTGTTGTTACGTTATGATACGCTGGCA | |
| | | CAGCGTCCTGCACAAGCCATGGAACAGGTATATGCATTCCTGCAGC | |
| | | TCCCTGCCTTTGCACATGATTATGCCGGTGTTCAGGCCGAAGCGGA | |
| | | ACGCTTTGATGCCGCCCTGCAAATGCCTGGTTTGCACCGCGTGCGT | |
| | | CGTGGTGTTCACTATGTTCCGCGACGTTCGGTTTTACCGCCTGCCC | |
| | | TGTTTGACCAGCTGCAGGAACTTGCATTCTGGGAAAGTGCACCCAG | |
| | | CCATGGAGCGCTGCTCGTG | |
| sgbL | Codon | ATGACAAGCCATGCAACCGAGGTTGAATGGGAGGACCTTCTGCGCC | 515 |
| | optimized | AAGCATTACACGCAACTGGTACAGGTGCTCGTTGGGCTGTAGAGGC | |
| | | GGACGAGATGTGGTGCCGTGTCGCCCCGGTGCCTGGAACTCGCCGC | |
| | | GAGCAAGGATGGAAGCTTCATGTAAGCGCGACGACCGCGAGTGCGC | |
| | | CCGAAGTCTTAACTCGTGCATTAGGCGTACTTCTGCGTGAAAAGTC | |
| | | CGGGTTCAAATTTGCCCGCTCACTTGAACAAGTCTCGGCCTTGAAT | |
| | | AGTCGTGCTACGCCCCGTGGTAGTTCGGGTAAATTTATCACAGTAT | |
| | | ACCCCCGCTCAGACGCCGAAGCCGTCGCACTGGCTCGCGACCTGCA | |
| | | TGCGGCAACGGCCGGCTTGGCTGGGCCCCGTATTCTTTCCGATCAA | |
| | | CCATACGCCGCGCACAGCCTGGTGCATTATCGTTATGGGGCTTTCG | |
| | | TGGGACGTCGTCGCCTTTCAGATGACGGGCTTTTAGTTTGGTTTAT | |
| | | TGAGGACCCAGATGGCAATCCCGTGGAGGATAAACGCACCGGACGT | |
| | | TATGCGCCGCCTCCCTGGGCTGTATGTCCGTTTCCTGCGAGCGTCC | |
| | | CCGTTGCGCCCCATGACGGCGAAGCTACGAGTCGTCCTGTTGTCTT | |
| | | AGGTGGTCGCTTCGCGGTTCGTGAAGCCATCCGTCAAACGAATAAA | |
| | | GGGGGCGTCTATCGCGGGTCGGACACACGCACTGGCACCGGCGTGG | |
| | | TTATCAAAGAGGCGCGCCCACATGTTGAAGGAGACGCCAGTGGGGG | |
| | | CGATGTTCGTGACTGGCTTCGCGCAGAGGCGCGTACGCTTGAAAAA | |
| | | TTAAAAGGTACCGGCTTGGCACCAGAAGCGGTGGCGTTGTTTGAGC | |
| | | ACGCTGGCCACTTGTTCTTAGCCCAAGACGAGGTCCCGGGGGTTAC | |
| | | GTTACGCACCTGGGTAGCGGAACACTTCCGTGACGTTGGAGGAGAG | |
| | | CGCTATCGTGCCGACGCCCTGGCTCAGGTGGCTCGTTTAGTTGATT | |
| | | TAGTCGCGGCTGCTCATGCACGTGGCTTGGTCCTGCGCGATTTTAC | |
| | | ACCAGGGAACGTGATGGTCCGTCCAGACGGCGAATTGCGCCTTATT | |
| | | GATTTAGAGCTGGCGGTTCTTGAGGATGAGGCCGCATTGCCTACCC | |
| | | ACGTCGGTACCCCGGGGTTTTCGGCACCCGAACGCCTTGCAGACGC | |
| | | TCCAGTGCGTCCTACTGCTGACTACTATTCTCTGGGAGCCACAGCT | |
| | | TGTTTTGTCTTGGCCGGTAAAGTCCCTAATTTACTTCCTGAAGAAC | |
| | | CCGTGGGTCGCCCATCGGAGGAGCGTCTTGCTGCCTGGTTGACTGC | |
| | | ATGTACACGTCCGCTGCGCCTGCCAGATGGAGTCGTTGACATGATC | |
| | | TTGGGGTTAATGCGCGATGATCCTGCAGAGCGCTGGGACCCATCCC | |
| | | GCGCGCGTGAAGCACTGCGCAAAGCTGACCCGACAGCACGCCCCGG | |
| | | GGATGCTGATCGCACTGCAGTACGTCGTACGGGTTCGTCGGCAGTG | |
| | | GCCGGGCCAGTTCCTGACTCACGTACAGCAGATGGTCGTACAGCGG | |
| | | ACGGCCGTTCCGCGGATGAAGTTGTGGCAGGTCTTGTCGATCACTT | |
| | | AGTCGATAGTATGACCCCGGCAGATGATCGTCTGTGGCCGGTAAGC | |
| | | ACTCTTACGGGAGAATCGGATCCATGTACAGTCCAGCAAGGCGCTG | |
| | | CTGGGGTGCTTGCGGTGTTGACCCGCTACTTCGAATTGACGGGCGA | |
| | | TCCGCGCTTACCAGGCTTATTGTCGACAGCCGGACGTTGGATCGCA | |
| | | GACCGCACGGATGTTCGTTCACCTCGTCCGGGATTACATTTCGGGG | |
| | | GACGCGGAACAGCCTGGGCCTTATACGACGCGGGGCGTGCAGTCGA | |
| | | CGATCGTCGCTTGGTGGAACATGCTCTGGACTTAGCATTAGCCCCG | |
| | | CCCCAAGCGACTCCTCATCACGATGTCACGCATGGGACTGCGGGCT | |
| | | CAGGCTTAGCCGCCTTGCACCTGTGGCAGCGTACTGGAGATACTCG | |
| | | TTTCGCGGATTTAGCAGTAGAGGCAGCTGATCGCTTAACAGCTGCA | |
| | | GCTCGTCGCGAGCCTTCGGGTGTTGGATGGGCAGTACCTGCAGAGG | |
| | | CCGACTCCCCAGAAGGAGGCAAGCGTTACCTGGGCTTCGCTCATGG | |
| | | CGCAGCTGGGATTGGGTGCTTCTTATTGGCTGCGGCGGAACTTAGT | |
| | | CGTCAACCCGATCATCGTGCAACTGCTTTGGAAGTTGGCGAAGGCC | |
| | | TGGTTGCTGATGCTGTTCGCATCGGAGAGGCGGCACAGTGGCCTGC | |
| | | GCAATCCGGGGACTTGCCGACAGCGCCTTACTGGTGCCATGGGGCG | |
| | | GCAGGTATCGGGACATTTCTTGTACGCTTATGGCAGGCGACCGGGG | |
| | | ACGATCGCTTCGGTGATCTGGCCCGCGGGAGTGCTCACGCTGTGGC | |
| | | CGAACGTGCTAGTCGCGCCCCATTGGCGCAATGTCACGGTTTGGCT | |
| | | GGAAACGGAGATTTCTTGTTGGATTTGGCAGACGCGACAGGCGATC | |
| | | CTGTGCATCGCGACACCGCGGAAGAGTTAGCAGGGTTGATCTTGGC | |
| | | CGAAGGAACCCGTCGTCAGGGACATGTCGTTTTCCCTAATGAGTAT | |
| | | GGGGAAGTATCATCTTCATGGTCCGACGGTAGTGCGGGGATTCTTG | |
| | | CGTTCCTTCTGCGTACGCGTCATACGGGCCCTCGCCATTGGATGGT | |
| | | AGAACAACGTGGG | |
| stspM | Codon | ATGGCGGATCATATTGCGGCCGGTCATGACACCGTCCTGAGCCTGG | 516 |
| | optimized | CCGAACGGACAGGTACCGATCCAGATCTGCTGGGCCGTGTGTTGCG | |
| | | CTTCCTCGCTTGTCGTGGTGTTTTCGCCGAGCCTCGCCCAGGTACT | |
| | | TATGCCTTGACCCCTCTGAGCTTAACTTTACTGGAAGGCCATCCGT | |
| | | CCGGTTTAAGAGAATGGTTGGATGCGTCGGGTGCGGGAGCGCGCAT | |
| | | GGACGCGGCAGTTGGAGATCTGCTTGGCGCCCTCCGCTCGGGTGAA | |
| | | CCGAGCTATCCACGTCTGCATGGTCGTCCGTTTTATGAAGATCTGG | |
| | | CGCTGCACAGCCGAGGCCCTGCTTTTGATGGACTGCGTCATACGCA | |
| | | CGCCGAATCGTATGTTGCCGACCTGCTGGCAGCCTACCCGTGGGAA | |
| | | CGCGTTCGTCGCGTGGTTGATGTAGGCGGTGGGACCGGCGTATTGG | |
| | | TCGAGGCGCTTATGAGAACTCATGCGACCCTCCGTACAGTACTGGT | |
| | | CGATCTTCCAGGCGCGGTGGCTACCGCTACCGCTCGAATTGCGGCT | |
| | | GCGGGTTTTGGCAATAGATATACACCGGTCACGGGTTCCTTCTTTG | |
| | | ATCCGCTGCCTGCGGGGGCGGATGTTTACACCCTGGTTAACGTGGT | |
| | | TCACAACTGGAACGATGAGCGTGCCTCAGCTCTGCTGCGTCGGTGT | |
| | | GCGGATGCGGGTCGCCGCGACAGTACGTTTGTTATCGTGGAACGCT | |
| | | TAGCGGACGATGCAGACCCTCGTGCCATCACCGCCATGGACCTCCG | |
| | | TATGTTCCTTTTTCTGGGCGGTAAAGAGCGCACGGCCGCACAGATT | |
| | | CGCGAAGTAGCTAGTGCGGCTGGCATGGCCCACCAAAGCACCATTA | |
| | | AAACACCGTCTGGCCTCCACTTACTTGTTTTCCGTAAGAAACGTTT | |
| | | CGCTGCTCGCGGTCACGGTCGTCGCATGGTGACC | |
| tgnB | Codon | ATGAAAACCATTCTGATTATTACCAATACCCTGGATCTGACCGTGG | 517 |
| | optimized | ATTATATTATTAATCGCTATAATCATACCGCTAAATTTTTTCGTCT | |
| | | GAATACCGATCGTTTTTTTGATTATGATATTAATATTACCAATAGC | |
| | | GGTACCAGCATTCGTAATCGTAAATCTAATCTGATTATTAATATTC | |
| | | AGGAAATTCATAGCCTGTATTATCGCAAAATTACCCTGCCGAATCT | |
| | | GGATGGCTATGAAAGTAAATATTGGACCCTGATGCAGCGCGAAATG | |
| | | ATGAGTATTGTTGAAGGCATTGCAGAAACCGCTGGCAATTTTGCAC | |
| | | TGACCCGTCCGTCTGTGCTGCGCAAAGCTGATAATAAAATTGTGCA | |
| | | GATGAAACTGGCAGAAGAAATTGGTTTTATTCTGCCGCAGAGTCTG | |
| | | ATTACCAATTCAAATCAGGCGGCAGCCTCATTTTGCAATAAAAATA | |
| | | ATACCAGCATTGTGAAACCGCTGAGTACCGGCCGCATTCTGGGTAA | |
| | | AAATAAAATTGGCATTATTCAGACCAATCTGGTTGAAACCCATGAA | |
| | | AATATTCAGGGCCTGGAACTGTCTCCGGCTTATTTTCAGGATTATA | |
| | | TTCCGAAAGATACCGAAATTCGTCTGACCATTGTTGGTAATAAACT | |
| | | GTTTGGCGCCAATATTAAATCAACCAATCAGGTTGATTGGCGCAAA | |
| | | AATGATGCACTGCTGGAATATAAACCGGCCAATATTCCGGATAAAA | |
| | | TTGCCAAAATGTGTCTGGAAATGATGGAAAAACTGGAAATTAATTT | |
| | | TGCGGCGTTTGATTTTATTATTCGTAATGGTGATTATATTTTTCTG | |
| | | GAACTGAATGCCAATGGTCAGTGGCTGTGGCTGGAAGATATTCTGA | |
| | | AATTTGATATTTCAAATACCATTATTAATTATCTGCTGGGTGAACC | |
| | | GATTTAA | |
| thcoK | Codon | ATGACGAGAACCAACACCGGCTATCGTTATCGCGCGTTCGGCCTGC | 518 |
| | optimized | GCATAGACTCAGATATTCCGCTGCCAGAATTAGGGGACGGTACGCG | |
| | | CCCTGATGGTGACGCGGATCTGACGGTCGTCCGGTGTGGGGAAGCG | |
| | | GAGCCGGAATGGGCTGAAGGTGGTGGCGGGGGTCGTCTGTATGCCG | |
| | | CTGAAGGCATTGTATCTTTTCGCGTGCCGCAGACGGCAGCGTTCCG | |
| | | TATTACTAATGGAAATCGCATCGAGGTGCATGCCTACTCGGGGGCT | |
| | | GATGAGGATCGAATACGCCTGTACGTGTTAGGGACCTGTATGGGAG | |
| | | CGCTGTTACTGCAACGTAGAATCTTACCGCTTCATGGTTCGGTCGT | |
| | | CGCCCGTGATGGTCGTGCGTATGCCATAGTTGGCGAAAGCGGAGCG | |
| | | GGCAAATCCACGATGAGTGCAGCACTTCTCGAACGTGGATTCCGCC | |
| | | TCGTTACGGATGACGTGGCCGCCATCGTGTTCGATGAGCGTGGGAC | |
| | | CCCACTGGTTATGCCGGCTTATCCACAGCAAAAACTGTGGCAGGAT | |
| | | TCCCTGGACCGTCTGCAAATTGCGGGCTCGGGCCTTCGTCCGCTGT | |
| | | TCGAACGCGAAACGAAATACGCTGTACCCGCGGATGGGGCATTCTG | |
| | | GCCCGAACCGGTTCCATTGGTGCACATTTACGAACTGGTTCATAGC | |
| | | GATGGTCAAACGCCTGAACTGCAGCCGATTGCCAAATTAGAGCGTT | |
| | | GCTATACCTTGTATCGCCACACATTTCGTAGAAGCCTGATCGTCCC | |
| | | CAGCGGCTTAAGCGCCTGGCATTTTGAAACGGCAGTGAAACTTGCG | |
| | | GAGAAAACGGGGATGTACCGTCTTATGCGCCCGGCCAAAGTTTTCG | |
| | | CGGCTCGCGAATCTGCTCGGCTGATTGAAACTCACGCCGATGGTGA | |
| | | AGTGTCACGT | |
| truD | Amplified | ATGCAACCAACCGCCCTCCAAATTAAGCCCCACTTCCACGTTGAGA | 519 |
| | from Topo-El | TAATTGAGCCGAAGCAAGTGTATCTCCTGGGCGAACAGGGCAACCA | |
| | | CGCTCTCACCGGGCAGCTCTACTGCCAAATTCTGCCTTTCTTAAAC | |
| | | GGCGAATACACCCGAGAACAAATTGTGGAAAAGCTCGATGGGCAGG | |
| | | TCCCGGAGGAATATATCGACTTCGTACTCAGTCGTCTGGTGGAGAA | |
| | | GGGCTATCTAACTGAGGTGGCTCCAGAACTATCCCTGGAAGTGGCA | |
| | | GCATTTTGGAGCGAATTGGGAATTGCCCCTTCTGTAGTGGCAGAAG | |
| | | GGCTAAAGCAGCCAGTGACAGTGACAACGGCGGGCAAGGGCATTAG | |
| | | GGAAGGGATAGTGGCTAACCTGGCAGCAGCGCTGGAGGAAGCTGGC | |
| | | ATTCAGGTGTCAGACCCAAGGGACCCAAAGGCCCCAAAGGCAGGGG | |
| | | ATTCTACTGCCCAGCTTCAGGTGGTGCTGACCGATGACTATTTACA | |
| | | GCCGGAACTTGCAGCGATCAACAAGGAAGCCTTAGAGCGCCAACAA | |
| | | CCCTGGTTGCTGGTTAAGCCTGTGGGCAGTATCCTCTGGTTGGGAC | |
| | | CGTTGTTCGTTCCTGGGGAAACCGGATGTTGGCACTGTCTTGCTCA | |
| | | ACGATTGCAAGGCAACCGGGAAGTTGAAGCATCGGTATTGCAACAA | |
| | | AAGCGAGCGCTGCAGGAGCGCAACGGTCAAAATAAAAATGGTGCAG | |
| | | TGAGTTGCTTGCCCACAGCACGGGCAACCCTACCTTCTACTCTACA | |
| | | AACAGGTTTACAGTGGGCTGCCACTGAGATTGCTAAGTGGATGGTC | |
| | | AAGCGGCACCTCAATGCCATAGCACCGGGAACGGCTCGTTTTCCCA | |
| | | CTCTAGCTGGCAAGATATTTACATTCAACCAGACGACTCTGGAGTT | |
| | | GAAAGCTCATCCTCTGAGCCGACGACCGCAATGTCCCACCTGTGGC | |
| | | GATCGGGAAACTCTCCAACGGCGCGGGTTTGAACCACTGAAGCTAG | |
| | | AGTCGCGCCCCAAACACTTCACCTCCGATGGCGGTCATCGCGCCAT | |
| | | GACCCCAGAACAAACGGTGCAGAAGTACCAACACCTCATCGGGCCC | |
| | | ATAACGGGGGTAGTGACGGAACTGGTGCGAATTTCTGACCCTGCCA | |
| | | ATCCCTTGGTGCATACCTACCGGGCTGGGCATAGCTTTGGCAGTGC | |
| | | TACGTCTCTGCGGGGGCTGCGCAATGTCCTACGCCACAAGAGTTCT | |
| | | GGTAAAGGCAAGACCGATAGCCAATCTCGGGCCAGCGGACTTTGCG | |
| | | AGGCGATCGAGCGCTATTCGGGCATTTTTCAGGGAGACGAACCCCG | |
| | | CAAGCGGGCAACTTTGGCTGAGTTGGGAGATTTGGCGATTCATCCA | |
| | | GAACAGTGTTTGCACTTTAGCGACAGGCAGTATGACAACCGGGAAA | |
| | | GCTCGAACGAGCGAGCAACAGTGACTCACGACTGGATTCCCCAACG | |
| | | GTTCGATGCAAGTAAGGCTCACGACTGGACTCCCGTGTGGTCCCTA | |
| | | ACGGAGCAAACCCATAAGTATCTGCCTACAGCCCTGTGCTATTACC | |
| | | GATACCCCTTCCCCCCAGAACACCGTTTCTGCCGTAGTGACTCCAA | |
| | | CGGAAACGCGGCGGGAAATACCCTGGAAGAGGCGATTTTGCAAGGA | |
| | | TTTATGGAACTGGTGGAACGGGATAGCGTGTGCCTGTGGTGGTACA | |
| | | ATCGCGTTAGCCGTCCGGCTGTGGATTTGAGTAGCTTTGACGAGCC | |
| | | TTATTTTTTGCAGTTGCAGCAGTTCTATCAAACTCAAAATCGCGAT | |
| | | CTGTGGGTACTGGATTTAACAGCAGATTTGGGCATTCCGGCTTTTG | |
| | | TAGGGGTATCGAATCGGAAAGCCGGCAGCTCGGAAAGAATAATTCT | |
| | | CGGCTTTGGAGCGCACCTGGACCCGACAGTTGCCATCCTTCGCGCT | |
| | | CTTACGGAGGTCAACCAAATAGGCTTGGAATTGGATAAAGTTTCTG | |
| | | ATGAGAGCCTCAAGAACGATGCCACGGATTGGTTAGTGAATGCTAC | |
| | | ATTGGCAGCTAGTCCCTATCTCGTTGCCGATGCTAGCCAACCCCTC | |
| | | AAGACTGCGAAGGATTATCCCCGGCGTTGGAGTGACGATATTTACA | |
| | | CCGATGTGATGACTTGTGTAGAAATAGCCAAGCAAGCAGGTCTAGA | |
| | | GACTTTGGTACTGGATCAGACCAGACCCGACATAGGTTTAAATGTG | |
| | | GTTAAAGTCATTGTGCCAGGAATGCGTTTTTGGTCGCGATTTGGCT | |
| | | CCGGTCGGCTCTATGACGTGCCAGTGAAGTTGGGATGGCGAGAGCA | |
| | | ACCACTTGCTGAGGCACAAATGAACCCTACACCGATGCCATTT | |

Precursor peptides

| | | | SEQ |
|---|---|---|---|
| Name | Details | Sequence | ID NO: |
| albsA | Codon | ATGGATTCACTGCTGTCAACAGAAACCGTCATTAGTGATGACGAAC | 520 |
| | optimized | TGCTTCCGATTGAAGTTGGTGGTACCGCGGAATTGACAGAGGGGCA | |
| | | GGGCGGCGGTCAGTCCGAGGATAAACGTCGCGCTTATAACTGC | |
| amdnA | Codon | ATGCCGGAAAATCGGCAGGAAGATCTCAACGCTCAGGCTGTACCAT | 521 |
| | optimized | TCTTCGCGCGTTTCTTGGAGGGTCAAAACTGCGAGGACCTTACTGA | |
| | | TGAGGAATCGGAGGCGGTTAGCGGTGGAAAACGCGGCCAAACCCGT | |
| | | AAATATCCAAGCGACTGCGAAGATGGGAATGGCGTGACCGGTAAAC | |
| | | TGCGCGATGAAGATATTGCAGTGACCTTGAAGTACCCATCCGACAA | |
| | | TGAAGATAATGGCGGCGGTGAAATTGTGACTCTGAAGTTTCCAAGT | |
| | | GATGATGATGATCAACCAGTAGGC | |
| atxA1 | Codon | CCGATCATTAGCGAAACGGTCCAGCCTAAAACGGCTGGCCTGATTG | 522 |
| | optimized | TTCTGGGCAAGGCAAGCGCGGAAACGCGCGGATTGAGCCAAGGCGT | |
| | | GGAACCGGACATTGGTCAGACGTACTTCGAAGAAAGCCGTATTAAT | |
| | | CAGGAT | |
| bamA | Codon | CTGAAAATCCGCAAGGTGAAAATTGTCAGAGCGCAGAACGGCCACT | 523 |
| | optimized | ACACGAAC | |
| bmbC | Codon | ATGGGTCCGGTTGTTGTGTTCGATTGCATGACGGCCGACTTTCTGA | 524 |
| | optimized | ACGACGATCCAAATAACGCGGAGTTGTCTGCCTTGGAAATGGAGGA | |
| | | GCTCGAGTCCTGGGGCGCCTGGGACGGAGAGGCTACCAGC | |
| bsjA2 | Codon | ATGACCAATGAAGAGATCATTGTCGCGTGGAAAAACCCTAAAGTCC | 525 |
| | optimized | GTGGCAAAAATATGCCAAGTCACCCGAGCGGCGTGGGATTCCAAGA | |
| | | GCTTTCCATCAACGAGATGGCCCAAGTGACCGGCGGAGCAGTAGAA | |
| | | CAGCGTGCAACACCAACCCTGGCAACCCCGCTGACCCCGCATACCC | |
| | | CGTACGCAACCTATGTGGTTAGCGGAGGCGTGGTTAGCGCGATTTC | |
| | | TGGTATCTTCAGCAACAATAAAACGTGTCTGGGC | |
| bsjA3 | Codon | ATGACCAATGAGGAAATTATCGTTGCGTGGAAAAACCCGAAGGTGC | 526 |
| | optimized | GCGGCAAAAACATGCCTTCCCATCCGTCCGGTGTGGGCTTCCAGGA | |
| | | ATTATCTATTAATGAAATGGCACAGGTGACTGGTGGCGCGGTTGAA | |
| | | CAGCGCGCGACGCCGGCAACCCCAGCAACACCATGGCTGATTAAAG | |
| | | CGTCTTATGTGGTGAGTGGGGCGGGAGTTTCTTTTGTCGCAAGCTA | |
| | | TATCACTGTAAAC | |
| capA | Codon | ATGGTGCGTTTCCTGGCTAAGCTGCTGCGTTCAACGATCCATGGCT | 527 |
| | optimized | CTAATGGCGTGAGCCTCGACGCCGTCAGTTCCACGCATGGTACTCC | |
| | | GGGGTTTCAGACACCTGATGCACGTGTTATTTCACGCTTTGGCTTT | |
| | | AAT | |
| cinA | Codon | ATGACGGCGAGTATTCTTCAGTCTGTCGTTGATGCGGACTTTCGTG | 528 |
| | optimized | CGGCCCTGATTGAAAACCCAGCCGCATTCGGCGCGAGCACCGCAGT | |
| | | TTTGCCGACCCCAGTCGAACAGCAGGATCAGGCATCACTGGATTTT | |
| | | TGGACAAAAGATATTGCTGCCACTGAGGCGTTTGCTTGCAAACAGT | |
| | | CTTGCTCATTTGGGCCGTTCACCTTTGTGTGCGACGGGAATACCAA | |
| | | A | |
| cln1A1 | Codon | ACTCCCATTCAATCCAAATTCTGCCTCCTGCGCGTGGGCAGTGCCA | 529 |
| | optimized | AACGGCTGACGCAGTCATTCGACGTGGGAACTATTAAGGAAGGTTT | |
| | | AGTCAGCCAGTATTATTTTGCG | |
| cln1A2 | Codon | ACCCAGGTGAGCCCATCACCGCTGCGCCTGATTCGCGTCGGGAGAG | 530 |
| | optimized | CCTTGGACCTGACCCGCTCTATCGGGGATAGTGGGCTGCGTGAGTC | |
| | | CATGTCAAGCCAGACGTACTGGCCC | |
| cln2A1 | Codon | AACACTTTAAAAACGCGTCTTATTCGCTTTGGGTCGGCTAAACGTC | 531 |
| | optimized | TGACGCGCGCAGGTACGGGCGTGCTGTTACCTGAAACCAACCAGAT | |
| | | TAAGCGCTACGATCCAGCA | |
| cln2A2 | Codon | ACCACACCCAAATTTCGACTGATTCGGTTAGGTTCAGCTAAGCGAT | 532 |
| | optimized | TGACCCGGTCGGGAATCGGGGATGTGTTTCCGGAGCCAAACATGGT | |
| | | TCGCCGCTGGGAT | |
| cln3A1 | Codon | CAGCGTATAATAGATGAAACCACCGATGGTCTGATTGAACTGGGGG | 533 |
| | optimized | CGGCCAGCGTACAGACACAGGGCGATGTTTTGTTTGCTCCGGAGCC | |
| | | TGGCGTGGGCCGACCTCCAATGGGCCTTTCCGAAGAT | |
| cln3A2 | Codon | GAACGCATTGAAGATCATATTGATGATGAACTGATTGACCTGGGAG | 534 |
| | optimized | CTGCTTCGGTTGAAACCCAGGGAGATGTGCTGAATGCACCGGAGCC | |
| | | TGGTATCGGTCGTGAACCGACAGGCTTGAGCCGCGAT | |
| cln3A3 | Codon | GAATTTGAAGGTATCCCATCACCGGATGCGCGTATTGATTTGGGTC | 535 |
| | optimized | TGGCGTCGGAAGAAACCTGTGGTCAGATTTATGATCACCCGGAAGT | |
| | | AGGCATCGGTGCGTACGGGTGCGAGGGCCTGCAGCGT | |
| comX | Codon | CAAGATCTGATTAATTACTTCCTGAATTATCCTGAGGCTCTGAAGA | 536 |
| | optimized | AACTCAAGAATAAGGAAGCCTGCTTAATTGGGTTTGACGTCCAGGA | |
| | | AACCGAAACGATTATCAAAGCCTATAACGATTACTACCGCGCTGAT | |
| | | CCGATCACGCGTCAATGGGGTGAT | |
| crnA1 | Codon | ATGTCCGAACTGAGTATGGAGAAAGTGGTCGGCGAAACATTTGAGG | 537 |
| | optimized | ATCTGAGCATCGCGGAAATGACGATGGTGCAGGGCAGCGGCGACAT | |
| | | TAACGGCGAATTTACTACCTCGCCGGCATGTGTTTATTCCGTTATG | |
| | | GTTGTATCGAAAGCAAGCAGCGCTAAATGTGCGGCCGGTGCATCGG | |
| | | CAGTCTCGGGAGCCATTCTGAGTGCGATTCGTTGC | |
| crnA2 | Codon | ATGAGCGAATCCAACATGAAGAAGGTTGTTGGCGAAACCTTCGAAG | 538 |
| | optimized | ATCTGAGCATCGCAGAAATGACGAAAGTTCAGGGCTCAGGGGACGT | |
| | | GATGCCGGAATCTACCCCAATTTGTGCCGGCTTCGCAACCTTGATG | |
| | | AGTTCTATCGGTCTTGTTAAAACCATCAAAGGCAATGTCAAAAGTT | |
| | | TCTCCGTCTTAATT | |
| csegA1 | Codon | ACCAAGAAAAACGCAACACAGGCCCCACGTTTAGTACGTGTAGGCG | 539 |
| | optimized | ATGCTCATCGTTTGACCCAAGGTGCTTTCGTTGGACAGCCGGAAGC | |
| | | CGTAAATCCACTTGGACGTGAAATTCAAGGA | |
| csegA2 | Codon | ACCAAAACACACAGACTGATCAGATTGGGCGACGCGCAACGCTTGA | 540 |
| | optimized | CCCAGGGCACATTGACTCCGGGCTTACCGGAGGACTTTCTGCCGGG | |
| | | CCATTACATGCCGGGG | |
| csegA3 | Codon | ACTTCACGTTTCCAACTCCTGCGCCTGGGAAAAGCCGATCGTTTGA | 541 |
| | optimized | CGCGTGGCGCGCTGGTCGGGCTCCTGATCGAAGATATTACTGTCGC | |
| | | TCGCTACGACCCTATG | |
| epiA | Codon | GAAGCAGTTAAAGAGAAGAACGATCTGTTCAACCTGGATGTTAAAG | 542 |
| | optimized | TCAACGCAAAAGAAAGTAACGATAGTGGCGCAGAACCACGCATAGC | |
| | | GTCGAAATTTATTTGCACACCAGGCTGCGCGAAAACGGGTTCGTTT | |
| | | AACAGCTATTGTTGT | |
| halA1 | Codon | ACGAACTTGCTGAAAGAATGGAAAATGCCCCTGGAACGTACGCATA | 543 |
| | optimized | ATAACTCCAACCCGGCGGGAGACATTTTTCAGGAACTGGAAGATCA | |
| | | AGACATACTCGCCGGTGTGAATGGAGCAGAAAACTTATACTTTCAG | |
| | | GGTTGTGCGTGGTATAACATTAGCTGCCGTCTGGGCAACAAAGGAG | |
| | | CCTACTGCACCCTTACAGTTGAGTGCATGCCCTCCTGTAAC | |
| halA2 | Codon | GTGAATTCCAAAGACCTGAGAAATCCAGAATTTCGCAAAGCTCAGG | 544 |
| | optimized | GTCTGCAGTTTGTAGATGAAGTTAATGAGAAGGAACTCTCGAGTTT | |
| | | AGCCGGCAGCGAGAATCTTTACTTTCAAGGCACGACGTGGCCATGT | |
| | | GCGACCGTCGGCGTTTCAGTTGCCTTGTGCCCGACGACCAAATGCA | |
| | | CTTCACAGTGC | |
| kgpE | Codon | AAGAACCCGACGCTGTTGCCCAAACTGACCGCGCCGGTCGAACGTC | 545 |
| | optimized | CGGCCGTAACTTCGTCGGATTTAAAGCAAGCCTCAAGCGTCGATGC | |
| | | TGCATGGTTAAATGGCGATAATAACTGGTCAACCCCATTCGCCGGT | |
| | | GTGAACGCGGCATGGTTAAATGGGGACAACAACTGGTCCACGCCTT | |
| | | TTGCGGGCGTGAATGCTGCATGGCTTAATGGCGACAATAACTGGAG | |
| | | CACTCCATTTGCCGCCGATGGCGCTGAG | |
| lasA | Codon | ATGGACAAACGTGTGCGTTACGAAAAACCGAGCCTGGTGAAAGAGG | 546 |
| | optimized | GTACGTTTCGCAAAACTACCGCTGGCCTGCGGCGTCTGTTCGCTGA | |
| | | CCAGCTGGTTGGCCGCCGTAACATT | |
| lcnA | Codon | ACTAAAGGCCTGGACAAAATGCTTTTAACCAAAAAGAAGAAGGATA | 547 |
| | optimized | GTATGGGTCTGCTGAACGAAATCGACGTTACCACCCTGGATGAACA | |
| | | GTTAGGCGGTAAAATGAGCAAAGCATGGTGCCGATCCATGGTGGTG | |
| | | TCCTGCGTGTATAACCTGGTTGATTTTTCGTCGTCGAGTGACGGGA | |
| | | AAAAGACATGTGCTCTGTACCGCAAATATTGT | |
| ltnA1 | Codon | ATGAATAAAAACGAAATCGAAACCCAGCCAGTTACGTGGCTGGAGG | 548 |
| | optimized | AAGTTTCTGATCAGAATTTTGATGAGGATGTCTTTGGTGCGTGTAG | |
| | | CACAAACACCTTCTCGCTGAGCGATTACTGGGGTAACAACGGTGCT | |
| | | TGGTGTACACTCACGCACGAATGTATGGCATGGTGCAAG | |
| ltnA2 | Codon | ATGAAGGAAAAGAATATGAAGAAAAACGACACCATCGAACTTCAGC | 549 |
| | optimized | TTGGAAAATACCTGGAAGATGATATGATCGAACTGGCTGAAGGGGA | |
| | | TGAGTCCCATGGGGGTACTACCCCGGCTACCCCTGCGATTTCTATC | |
| | | CTCAGCGCGTATATCAGCACCAATACCTGCCCGACAACTAAGTGTA | |
| | | CACGCGCGTGC | |
| mcbA | Synthesized, | ATGGAATTAAAAGCGAGTGAATTTGGTGTAGTTTTGTCCGTTGATG | 550 |
| | sequence from | CTCTTAAATTATCACGCCAGTCTCCATTAGGTGTTGGCATTGGTGG | |
| | genome | TGGTGGCGGCGGCGGCGGCGGCGGCGGTAGCTGCGGTGGTCAAGGT | |
| | | GGCGGTTGTGGTGGTTGCAGCAACGGTTGTAGTGGTGGAAACGGTG | |
| | | GCAGCGGCGGAAGTGGTTCACATATC | |
| mdnA | Amplified | ATGGCATATCCCAACGATCAACAAGGTAAAGCACTTCCTTTCTTTG | 551 |
| | from | CTCGTTTCTTGTCCGTAAGCAAAGAGGAATCTTCCATCAAGTCTCC | |
| | pARW071 | TTCCCCTGAGCCTACCTACGGGGGCACCTTTAAATACCCTTCTGAC | |
| | | TGGGAAGATTAT | |
| mdnA* | Amplified | ATGGCACTTCCTTTCTTTGCTCGTTTCTTGTCCGTAAGCAAAGAGG | 552 |
| | from mdnA | AATCTTCCATCAAGTCTCCTTCCCCTGAGCCTACCTACGGGGGCAC | |
| | | CTTTAAATACCCTTCTGACTGGGAAGATTAT | |
| mibA | Codon | ATGCCAGCCGATATTCTGGAGACTCGTACCAGCGAAACGGAGGACT | 553 |
| | optimized | TACTGGATCTTGACCTGAGCATCGGTGTAGAAGAAATCACCGCAGG | |
| | | CCCGGCAGTGACTTCTTGGTCACTGTGCACCCCTGGATGCACGAGT | |
| | | CCGGGCGGTGGCTCCAATTGTTCGTTCTGTTGC | |
| paaP | Codon | ATGATTAAATTTTCTACATTGTCTCAGCGCATCAGCGCCATCACGG | 554 |
| | optimized | AAGAAAACGCCATGTACACTAAGGGTCAAGTGATCGTATTGAGC | |
| padeA | Codon | AAAAAGCAATATAGCAAACCTAGCCTGGAGGTTCTGGACGTCCACC | 555 |
| | optimized | AGACCATGGCTGGCCCGGGCACTAGTACGCCAGACGCGTTTCAGCC | |
| | | AGATCCAGATGAAGATGTTCACTATGATTCG | |
| palA | Codon | AAAGATCTTCTGAAGGAACTGATGTATGAAGTAGACCTCGAAGAGA | 556 |
| | optimized | TGGAGAATCTTCAGGGTAGCGGGTACTCAGCCGCCCAGTGTGCCTG | |
| | | GATGGCGCTGAGCTGCGTCAATTACATCCCGGGAGTGGGATTCGGT | |
| | | TGTGGCGGCTACAGCGCATGTGAACTCTACAAGCGTTATTGT | |
| papA | Codon | ATGTTGAAACAGATCAATGTGATTGCTGGCGTAAAAGAGCCTATTC | 557 |
| | optimized | GCGCCTATGGTTGTTCGGCTAATGACGCATGCTATTTTTGCGACAC | |
| | | GCGTGACAACTGCAAAGCCTGTGATGCCAGTGATTTTTGTATCAAA | |
| | | AGTGATACG | |
| papA_tev | Codon | TTGAAACAGATCAATGTGATTGCTGGCGTAAAAGAGCCTATTCGCG | 558 |
| | optimized | CCTATGAGAACTTGTATTTCCAGGGTTGTTCGGCTAATGACGCATG | |
| | | CTATTTTTGCGACACGCGTGACAACTGCAAAGCCTGTGATGCCAGT | |
| | | GATTTTTGTATCAAAAGTGATACG | |
| papoA | Codon | AGCAAGAAAGAATGGCAAGAGCCCACGATCGAAGTGCTCGATATTA | 559 |
| | optimized | ATCAGACTATGGCGGGTAAGGGCTGGAAACAGATAGACTGGGTGAG | |
| | | CGACCATGATGCTGACTTACACAATCCGTCT | |
| pbtA | Codon | ATGAACCTGAACGATTTACCTATGGACGTCTTTGAAATGGCAGACA | 560 |
| | optimized | GCGGTATGGAGGTGGAAAGCCTCACGGCTGGCCATGGCATGCCAGA | |
| | | AGTTGGAGCTAGTTGCAACTGTGTGTGCGGGTTTTGCTGCAGCTGC | |
| | | AGTCCGAGCGCG | |
| pcpA | Codon | ATGTCGAGTAATATCCTCGAAAAAGTTAAGGAGTTTTTCGTCCGGC | 561 |
| | optimized | TGGTGAAGGATGATGCGTTTCAAAGCCAGCTGCAGAACAACAGTAT | |
| | | TGATGAAGTTCGAAATATCCTGCAGGAGGCCGGGTACATATTCAGC | |
| | | AAAGAAGAATTCGAAACCGCAACCATTGAATTGCTGGATTTGAAGG | |
| | | AACGCGATGAATTCCACGAGCTGACAGAAGAGGAGCTTGTCACCGC | |
| | | TGTTGGCGGTGTTACGGGCGGGAGTGGTATATATGGCCCGATTCAA | |
| | | GCTATGTACGGTGCCGTCGTAGGTGATCCAAAACCGGGTAAGGACT | |
| | | GGGGGTGGCGCTTTCCGAGCCCGCTGCCAAAACCGAGTCCGATTCC | |
| | | GAGTCCGTGGAAACCCCCGGTTGATGTCCAGCCTATGTATGGTGTG | |
| | | GTAGTGTCAAACGATAGT | |
| pgm2 | Codon | ATGGAGCGCGAAATCGTGTGGACAGAAATTGAGGAGTCGGATTTAG | 562 |
| | optimized | CCGCCGTCGTGTCGGCATCTAATGTCAAGGATGGTCCAACCGTTAG | |
| | | CTCAAGTAATGTAAAGGACCGC | |
| plpA1 | Codon | ATGAGCATTGAGAATGCCAAGAGCTTTTATGAACGCGTCAGTACAG | 563 |
| | optimized | ATAAGCAGTTCCGCACTCAACTGGAAAATACGGCCAGTGCTGAAGA | |
| | | ACGGCAGAAAATCATTCAGGCAGCGGGCTTTGAGTTCACCAATCAG | |
| | | GAGTGGGAAATTGCAAAAGAACAGATTCTTGCGACAAGTGAAAGTA | |
| | | ATAACGGTGAACTGTCCGAGGCCGAACTGACCGCCGTCAGCGGTGG | |
| | | GGTTGACTTAAGCATTTTCGAGCTGCTGGACGAAGAACCTTTATTC | |
| | | CCGATTCGTCCTTTGTACGGCCTGCCTATT | |
| plpA2 | Codon | ATGTCTATTGAGAGTGCAAAGGCTTTCTACCAGCGTATGACGGATG | 564 |
| | optimized | ACGCATCTTTTCGTACCCCTTTTGAAGCGGAACTGTCGAAAGAGGA | |
| | | GCGCCAACAATTAATCAAAGATAGCGGATATGACTTTACTGCAGAA | |
| | | GAATGGCAACAGGCTATGACCGAGATCCAGGCGGCACGCTCAAACG | |
| | | AGGAACTGAATGAGGAAGAACTCGAGGCAATTGCCGGGGGCGCTGT | |
| | | GGCCGCAATGTATGGTGTGGTTTTCCCATGGGACAACGAGTTCCCG | |
| | | TGGCCCCGCTGGGGCGGT | |
| pqqA | Amplified | ATGTGGAAGAAACCTGCTTTTATCGATTTACGTCTCGGTCTGGAAG | 565 |
| | from genome | TGACGCTGTACATTTCTAACCGT | |
| procA* | Codon | ATGTCAGAAGAACAACTCAAGGCATTCATTGCCAAGGTTCAAGCAG | 566 |
| | optimized | ACACTTCACTGCAGGAACAGCTCAAAGTAGAAGGTGCTGATGTTGT | |
| | | TGCTATTGCTAAAGCCTCAGGGTTCGCGATTACCACAGAGGACCTC | |
| | | AATTCGCATCGCCAAAATCTGTCTGATGATGAGCTGGAGGGAGTCG | |
| | | CGGGAGGCTTTTTCTGCGTACAGGGTACGGCCAACCGTTTCACTAT | |
| | | CAACGTTTGC | |
| procA1.7 | Codon | ATGTCAGAAGAACAACTCAAGGCATTCATTGCCAAGGTTCAAGCAG | 567 |
| | optimized | ACACTTCACTGCAGGAACAGCTCAAAGTAGAAGGTGCTGATGTTGT | |
| | | TGCTATTGCTAAAGCCTCAGGGTTCGCGATTACCACAGAGGACTTA | |
| | | AAAGCACATCAAGCCAACTCACAAAAGAACCTGTCTGATGCTGAGC | |
| | | TGGAAGGTGTGGCTGGGCGAACCATTGGGGGAACCATTGTGTCGAT | |
| | | AACCTGTGAGACTTGCGATCTGCTTGTGGGGAAAATGTGC | |
| psnA2 | Codon | ATGAGCAAAAATGAGAACAACAAGAAACAGCTGCGCGATCTTTTCA | 568 |
| | optimized | TTGAAGATCTGGGCAAAGTTACTGGCGGTAAAGGTGGCCCGTATAC | |
| | | CACCTTAGCCATTGGCGAAGAAGATCCGATTACCACTTTGGCTATC | |
| | | GGAGAAGAGGACCCTGATCCAACGACACTTGCCTTAGGTGAAGAGG | |
| | | ACCCAACTACGCTTGCAATCGGCGAAGAA | |
| psnA2_tev | Codon | ATGAGCAAAAATGAGAACAACAAGAAACAGCTGCGCGATCTTTTCA | 569 |
| | optimized | TTGAAGATCTGGGCAAAGTTACTGGCGAGAACTTGTATTTCCAGGG | |
| | | TAAAGGTGGCCCGTATACCACCTTAGCCATTGGCGAAGAAGATCCG | |
| | | ATTACCACTTTGGCTATCGGAGAAGAGGACCCTGATCCAACGACAC | |
| | | TTGCCTTAGGTGAAGAGGACCCAACTACGCTTGCAATCGGCGAAGA | |
| | | A | |
| raxX | Codon | AACCACTCTAAGAAAAGTCCGGCAAAAGGGGCAGCGTCCCTGCAGC | 570 |
| | optimized | GTCCTGCTGGGGCAAAAGGCCGCCCTGAACCTCTGGATCAACGCTT | |
| | | GTGGAAACACGTCGGTGGTGGTGACTACCCACCCCCAGGAGCCAAC | |
| | | CCAAAGCATGATCCACCACCCCGCAATCCGGGCCACCAT | |
| sboA | Amplified | ATGAAAAAAGCTGTCATTGTAGAAAACAAAGGTTGTGCAACATGCT | 571 |
| | from genome | CGATCGGAGCCGCTTGTCTAGTGGACGGTCCTATCCCTGATTTTGA | |
| | | AATTGCCGGTGCAACAGGTCTATTCGGTCTATGGGGA | |
| sgbA | Codon | TCTGGTCGCGGGCGCGATCCTGATGCTGCTGTACCTCCCTTGCCTC | 572 |
| | optimized | GTGTACCTCGCACTACTAATCATGAGCCACGTACGGCGTCCCGAGA | |
| | | ACCAAGAGCAGCTCCAAGAACTGGACCTACACGTCCGCCTTCGTCG | |
| | | CGTCCATCTCCGTGTGGTCACTCTCCTCAAACCCCTGGTGCAGGAC | |
| | | GCAGTGGATGTCGTGTGGAGCGTCAAAAATCGGCTGCGGCTTCGTC | |
| | | TGAGAAGGAAAAGACAATGGAGAACCAAGATTTGGAGTTATTAGCA | |
| | | CGCCTGCATGCACTTCCTGAGACTGAACCGGTGGGCGTCGACGGAT | |
| | | TACCCTATGGCGAGACTTGTGAGTGCGTCGGGTTACTTACGTTGTT | |
| | | GAACACCGTATGTATCGGCATTTCATGCGCT | |
| strA | Codon | ATGAGTAAGGAATTAGAAAAAGTTCTTGAATCCAGTTCAATGGCAA | 573 |
| | optimized | AGGGGGACGGCTGGAAGGTTATGGCTAAAGGTGACGGTTGGGAG | |
| stspA | Codon | AAGAAATTCTATGAAGCGCCAGCTCTCATCGAACGTGGCGCCTTTG | 574 |
| | optimized | CGGCTGCTACAGCGGGGTTTGGACGTCTGCTGGCGGATCAGCTGGT | |
| | | GGGACGCCTGATTCCG | |
| tbtA | Codon | ATGGACCTGAATGATCTGCCGATGGATGTTTTTGAACTGGCAGATA | 575 |
| | optimized | GCGGTGTTGCAGTTGAAAGCCTGACCGCAGGTCATGGTATGACCGA | |
| | | AGTTGGTGCAAGCTGTAATTGCTTTTGTTATATTTGTTGTAGCTGC | |
| | | AGCAGCGCC | |
| tfxA | Amplified | ATGGATAACAAGGTTGCGAAGAATGTCGAAGTGAAGAAGGGCTCCA | 576 |
| | from genome | TCAAGGCGACCTTCAAGGCTGCTGTTCTGAAGTCGAAGACGAAGGT | |
| | | CGACATCGGAGGTAGCCGTCAGGGCTGCGTCGCT | |
| tgnA* | Codon | TATCGACCTTATATTGCCAAGTATGTCGAAGAACAAACTCTGCAGA | 577 |
| | optimized | ATTCAACCAACCTGGTATATGACGACATCACGCAGATCTCTTTTAT | |
| | | CAATAAAGAAAAGAACGTGAAAAAAATTAATCTGGGTCCCGATACT | |
| | | ACGATCGTGACTGAAACCATCGAGAATGCGGACCCCGATGAGTATT | |
| | | TCTTA | |
| thcoA | Codon | CGCAAGAAAGAATGGCAGACACCAGAACTGGAAGTACTCGATGTAC | 578 |
| | optimized | GCCTCACCGCAGCGGGCCCGGGTAAAGCTAAACCGGATGCTGTGCA | |
| | | GCCAGACGAAGATGAAATAGTGCACTACTCA | |
| truE* | Codon | ATGAACAAGAAGAACATTTTACCGCAGTTAGGACAACCAGTCATCC | 579 |
| | optimized | GCCTTACTGCCGGTCAACTGTCAAGCCAACTGGCGGAGCTTTCTGA | |
| | | GGAGGCTCTGGGAGGGGTCGATGCCTCGTACGCGGTGTTCTGGCCG | |
| | | ATCTGTAGCTATGACGAC | |
| truE | Codon | ATGAACAAGAAGAACATTTTACCGCAGTTAGGACAACCAGTCATCC | 580 |
| | optimized | GCCTTACTGCCGGTCAACTGTCAAGCCAACTGGCGGAGCTTTCTGA | |
| | | GGAGGCTCTGGGAGTCGATGCCTCGACCTTGCCGGTTCCGACGTTG | |
| | | TGTAGCTATGACGGGGTGGACGCTAGCACAGTCCCTACACTTTGTA | |
| | | GTTACGATGAC | |
| truE_TEV | Codon | AACAAGAAGAACATTTTACCGCAGTTAGGACAACCAGTCATCCGCC | 581 |
| | optimized | TTACTGCCGGTCAACTGTCAAGCCAACTGGCGGAGCTTTCTGAGGA | |
| | | GGCTCTGGGAGAGAACTTGTATTTCCAGGGTGTCGATGCCTCGACC | |
| | | TTGCCGGTTCCGACGTTGTGTAGCTATGACGGGGTGGACGCTAGCA | |
| | | CAGTCCCTACACTTTGTAGTTACGATGAC | |

Plasmid origins

| | | | SEQ |
|---|---|---|---|
| Name | Details | Sequence | ID NO: |
| pSC101 | var2 - | AGTAAGACGGGTAAGCCTGTTGATGATACCGCTGCCTTACTGGGTG | 582 |
| | maintains at | CATTAGCCAGTCTGAATGACCTGTCACGGGATAATCCGAAGTGGTC | |
| | p15A-level | AGACTGGAAAATCAGAGGGCAGGAACTGCTGAACAGCAAAAAGTCA | |
| | copy number | GATAGCACCACATAGCAGACCCGCCATAAAACGCCCTGAGAAGCCC | |
| | | GTGACGGGCTTTTCTTGTATTATGGGTAGTTTCCTTGCATGAATCC | |
| | | ATAAAAGGCGCCTGTAGTGCCATTTACCCCCATTCACTGCCAGAGC | |
| | | CGTGAGCGCAGCGAACTGAATGTCACGAAAAAGACAGCGACTCAGG | |
| | | TGCCTGATGGTCGGAGACAAAAGGAATATTCAGCGATTTGCCCGAG | |
| | | CTTGCGAGGGTGCTACTTAAGCCTTTAGGGTTTTAAGGTCTGTTTT | |
| | | GTAGAGGAGCAAACAGCGTTTGCGACATCCTTTTGTAATACTGCGG | |
| | | AACTGACTAAAGTAGTGAGTTATACACAGGGCTGGGATCTATTCTT | |
| | | TTTATCTTTTTTTATTCTTTCTTTATTCTATAAATTATAACCACTT | |
| | | GAATATAAACAAAAAAAACACACAAAGGTCTAGCGGAATTTACAGA | |
| | | GGGTCTAGCAGAATTTACAAGTTTTCCAGCAAAGGTCTAGCAGAAT | |
| | | TTACAGATACCCACAACTCAAAGGAAAAGGACTAGTAATTATCATT | |
| | | GACTAGCCCATCTCAATTGGTATAGTGATTAAAATCACCTAGACCA | |
| | | ATTGAGATGTATGTCTGAATTAGTTGTTTTCAAAGCAAATGAACTA | |
| | | GCGATTAGTCGCTATGACTTAACGGAGCATGAAACCAAGCTAATTT | |
| | | TATGCTGTGTGGCACTACTCAACCCCACGATTGAAAACCCTACAAG | |
| | | GAAAGAACGGACGGTATCGTTCACTTATAACCAATACGCTCAGATG | |
| | | ATGAACATCAGTAGGGAAAATGCTTATGGTGTATTAGCTAAAGCAA | |
| | | CCAGAGAGCTGATGACGAGAACTGTGGAAATCAGGAATCCTTTGGT | |
| | | TAAAGGCTTTTGGATTTTCCAGTGGACAAACTATGCCAAGTTCTCA | |
| | | AGCGAAAAATTAGAATTAGTTTTTAGTGAAGAGATATTGCCTTATC | |
| | | TTTTCCAGTTAAAAAAATTCATAAAATATAATCTGGAACATGTTAA | |
| | | GTCTTTTGAAAACAAATACTCTATGAGGATTTATGAGTGGTTATTA | |
| | | AAAGAACTAACACAAAAGAAAACTCACAAGGCAAATATAGAGATTA | |
| | | GCCTTGATGAATTTAAGTTCATGTTAATGCTTGAAAATAACTACCA | |
| | | TGAGTTTAAAAGGCTTAACCAATGGGTTTTGAAACCAATAAGTAAA | |
| | | GATTTAAACACTTACAGCAATATGAAATTGGTGGTTGATAAGCGAG | |
| | | GCCGCCCGACTGATACGTTGATTTTCCAAGTTGAACTAGATAGACA | |
| | | AATGGATCTCGTAACCGAACTTGAGAACAACCAGATAAAAATGAAT | |
| | | GGTGACAAAATACCAACAACCATTACATCAGATTCCTACCTACATA | |
| | | ACGGACTAAGAAAAACACTACACGATGCTTTAACTGCAAAAATTCA | |
| | | GCTCACCAGTTTTGAGGCAAAATTTTTGAGTGACATGCAAAGTAAG | |
| | | TATGATCTCAATGGTTCGTTCTCATGGCTCACGCAAAAACAACGAA | |
| | | CCACACTAGAGAACATACTGGCTAAATACGGAAGGATCTGAGGTTC | |
| | | TTATGGCTCTTGTATCTATCAGTGAAGCATCAAGACTAACAAACAA | |
| | | AAGTAGAACAACTGTTCACCGTTACATATCAAAGGGAAAACTGTCC | |
| | | ATATGCACAGATGAAAACGGTGTAAAAAAGATAGATACATCAGAGC | |
| | | TTTTACGAGTTTTTGGTGCATTCAAAGCTGTTCACCATGAACAGAT | |
| | | CGACAATGTAACAGATGAACAGCATGTAACACCTAATAGAACAGGT | |
| | | GAAACCAGTAAAACAAAGCAACTAGAACATGAAATTGAACACCTGA | |
| | | GACAACTTGTTACAGCTCAACAGTCACACATAGACAGCCTGAAACA | |
| | | GGCGATGCTGCTTATCGAATCAAAGCTGCCGACAACACGGGAGCCA | |
| | | GTGACGCCTCCCGTGGGGAAAAAATCATGGCAATTCTGGAAGAAAT | |
| | | AGCGCTTTCAGCCGGCAAACCGGCTGAAGCCGGATCTGCGATTCTG | |
| | | ATAACAAACTAGCAACACCAGAACAGCCCGTTTGCGGGCAGCAAAA | |
| | | CCCGTAC | |
| p15A | | TTAATAAGATGATCTTCTTGAGATCGTTTTGGTCTGCGCGTAATCT | 583 |
| | | CTTGCTCTGAAAACGAAAAAACCGCCTTGCAGGGCGGTTTTTCGAA | |
| | | GGTTCTCTGAGCTACCAACTCTTTGAACCGAGGTAACTGGCTTGGA | |
| | | GGAGCGCAGTCACCAAAACTTGTCCTTTCAGTTTAGCCTTAACCGG | |
| | | CGCATGACTTCAAGACTAACTCCTCTAAATCAATTACCAGTGGCTG | |
| | | CTGCCAGTGGTGCTTTTGCATGTCTTTCCGGGTTGGACTCAAGACG | |
| | | ATAGTTACCGGATAAGGCGCAGCGGTCGGACTGAACGGGGGGTTCG | |
| | | TGCATACAGTCCAGCTTGGAGCGAACTGCCTACCCGGAACTGAGTG | |
| | | TCAGGCGTGGAATGAGACAAACGCGGCCATAACAGCGGAATGACAC | |
| | | CGGTAAACCGAAAGGCAGGAACAGGAGAGCGCACGAGGGAGCCGCC | |
| | | AGGGGAAACGCCTGGTATCTTTATAGTCCTGTCGGGTTTCGCCACC | |
| | | ACTGATTTGAGCGTCAGATTTCGTGATGCTTGTCAGGGGGGCGGAG | |
| | | CCTATGGAAAAACGGCTTTGCCGCGGCCCTCTCACTTCCCTGTTAA | |
| | | GTATCTTCCTGGCATCTTCCAGGAAATCTCCGCCCCGTTCGTAAGC | |
| | | CATTTCCGCTCGCCGCAGTCGAACGACCGAGCGTAGCGAGTCAGTG | |
| | | AGCGAGGAAGCGGAATATATCCTGTATCACATATTCTGCTGACGCA | |
| | | CCGGTGCAGCCTTTTTTCTCCTGCCACATGAAGCACTTCACTGACA | |
| | | CCCTCATCAGTGCCAACATAGTAAGCCAGTATACACTCCGCTA | |

TABLE 19
Plasmid Sequences

| | | | SEQ |
|---|---|---|---|
| | | | ID |
| Name^{a} | Description | Sequence^{b} | NO |
| pEG3017 | HIS₆-MBP- | CATTTCCCCGAAAAGTGCCACCTGACGTCTAAGAAACCATTATTATCATGACAT | 584 |
| | TruE* | TAACCTATAAAAATAGGCGTATCACGAGGCAGAATTTCAGATAAAAAAAATCCT | |
| | | TAGCTTTCGCTAAGGATGATTTCTGGAATTCGCGGCCGCTTCTAGAGGGCatcc | |
| | | cgaaaatttatcaaaaagagtattgacttaaagtctaacctataggatacttac | |
| | | 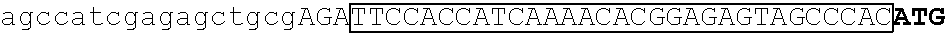 | |
| | | TCATATTACCACCATCACCATCATCACGACTATGATATTCCCACAAGCATGAAA | |
| | | ATCGAAGAAGGTAAACTGGTAATCTGGATTAACGGCGATAAAGGCTATAACGGA | |
| | | TTGGCTGAAGTCGGTAAGAAATTCGAGAAAGATACCGGAATTAAAGTCACCGTT | |
| | | GAGCATCCGGATAAACTGGAAGAGAAATTCCCACAGGTTGCGGCAACTGGCGAT | |
| | | GGCCCTGACATTATCTTCTGGGCACACGACCGCTTTGGTGGCTACGCTCAATCT | |
| | | GGCCTGTTGGCTGAAATCACCCCGGACAAAGCGTTCCAGGACAAGCTGTATCCG | |
| | | TTTACCTGGGATGCCGTACGTTACAACGGCAAGCTGATTGCTTACCCGATCGCT | |
| | | GTTGAAGCGTTATCGCTGATTTATAACAAAGATCTGCTGCCGAACCCGCCAAAA | |
| | | ACCTGGGAAGAGATCCCGGCGCTGGATAAAGAACTGAAAGCGAAAGGTAAGAGC | |
| | | GCGCTGATGTTCAACCTGCAAGAACCGTACTTCACCTGGCCGCTGATTGCTGCT | |
| | | GACGGGGGTTATGCGTTCAAGTATGAAAACGGCAAGTACGACATTAAAGACGTG | |
| | | GGCGTGGATAACGCTGGCGCGAAAGCGGGTCTGACCTTCCTGGTTGACCTGATT | |
| | | AAAAACAAACACATGAATGCAGACACCGATTACTCCATCGCAGAAGCTGCCTTT | |
| | | AATAAAGGCGAAACAGCGATGACCATCAACGGCCCGTGGGCATGGTCCAACATC | |
| | | GACACCAGCAAAGTGAATTATGGTGTAACGGTACTGCCGACCTTCAAGGGTCAA | |
| | | CCATCCAAACCGTTCGTTGGCGTGCTGAGCGCAGGTATTAACGCCGCCAGTCCG | |
| | | AACAAAGAGCTGGCGAAAGAGTTCCTCGAAAACTATCTGCTGACTGATGAAGGT | |
| | | CTGGAAGCGGTTAATAAAGACAAACCGCTGGGTGCCGTAGCGCTGAAGTCTTAC | |
| | | GAGGAAGAGTTGGCGAAAGATCCACGTATTGCCGCCACCATGGAAAACGCCCAG | |
| | | AAAGGTGAAATCATGCCGAACATCCCGCAGATGTCCGCTTTCTGGTATGCCGTG | |
| | | CGTACTGCGGTGATCAACGCCGCCAGCGGTCGTCAGACTGTCGATGAAGCCCTG | |
| | | AAAGACGCGCAGACTCGTATCACCAAGTCGTACTACCATCACCATCACCATCAC | |
| | | GGCGGTAGTGGCGAAAACCTGTATTTTCAGGGTATGAACAAGAAGAACATTTTA | |
| | | CCGCAGTTAGGACAACCAGTCATCCGCCTTACTGCCGGTCAACTGTCAAGCCAA | |
| | | CTGGCGGAGCTTTCTGAGGAGGCTCTGGGAGGGGTCGATGCCTCGTACGCGGTG | |
| | | TTCTGGCCGATCTGTAGCTATGACGACTAATAATTCAGCCAAAAAACTTAAGAC | |
| | | CGCCGGTCTTGTCCACTACCTTGCAGTAATGCGGTGGACAGGATCGGCGGTTTT | |
| | | CTTTTCTCTTCTCAACTGTACTAGTAGCGGCCGCTGCAGTCCGGCAAAAAAGGG | |
| | | CAAGGTGTCACCACCCTGCCCTTTTTCTTTAAAACCGAAAAGATTACTTCGCGT | |
| | | TATGCAGGCTTCCTCGCTCACTGACTCGCTGCGCTCGGTCGTTCGGCTGCGGCG | |
| | | AGCGGTATCAGCTCACTCAAAGGCGGTAATGACAGTAAGACGGGTAAGCCTGTT | |
| | | GATGATACCGCTGCCTTACTGGGTGCATTAGCCAGTCTGAATGACCTGTCACGG | |
| | | GATAATCCGAAGTGGTCAGACTGGAAAATCAGAGGGCAGGAACTGCTGAACAGC | |
| | | AAAAAGTCAGATAGCACCACATAGCAGACCCGCCATAAAACGCCCTGAGAAGCC | |
| | | CGTGACGGGCTTTTCTTGTATTATGGGTAGTTTCCTTGCATGAATCCATAAAAG | |
| | | GCGCCTGTAGTGCCATTTACCCCCATTCACTGCCAGAGCCGTGAGCGCAGCGAA | |
| | | CTGAATGTCACGAAAAAGACAGCGACTCAGGTGCCTGATGGTCGGAGACAAAAG | |
| | | GAATATTCAGCGATTTGCCCGAGCTTGCGAGGGTGCTACTTAAGCCTTTAGGGT | |
| | | TTTAAGGTCTGTTTTGTAGAGGAGCAAACAGCGTTTGCGACATCCTTTTGTAAT | |
| | | ACTGCGGAACTGACTAAAGTAGTGAGTTATACACAGGGCTGGGATCTATTCTTT | |
| | | TTATCTTTTTTTATTCTTTCTTTATTCTATAAATTATAACCACTTGAATATAAA | |
| | | CAAAAAAAACACACAAAGGTCTAGCGGAATTTACAGAGGGTCTAGCAGAATTTA | |
| | | CAAGTTTTCCAGCAAAGGTCTAGCAGAATTTACAGATACCCACAACTCAAAGGA | |
| | | AAAGGACTAGTAATTATCATTGACTAGCCCATCTCAATTGGTATAGTGATTAAA | |
| | | ATCACCTAGACCAATTGAGATGTATGTCTGAATTAGTTGTTTTCAAAGCAAATG | |
| | | AACTAGCGATTAGTCGCTATGACTTAACGGAGCATGAAACCAAGCTAATTTTAT | |
| | | GCTGTGTGGCACTACTCAACCCCACGATTGAAAACCCTACAAGGAAAGAACGGA | |
| | | CGGTATCGTTCACTTATAACCAATACGCTCAGATGATGAACATCAGTAGGGAAA | |
| | | ATGCTTATGGTGTATTAGCTAAAGCAACCAGAGAGCTGATGACGAGAACTGTGG | |
| | | AAATCAGGAATCCTTTGGTTAAAGGCTTTTGGATTTTCCAGTGGACAAACTATG | |
| | | CCAAGTTCTCAAGCGAAAAATTAGAATTAGTTTTTAGTGAAGAGATATTGCCTT | |
| | | ATCTTTTCCAGTTAAAAAAATTCATAAAATATAATCTGGAACATGTTAAGTCTT | |
| | | TTGAAAACAAATACTCTATGAGGATTTATGAGTGGTTATTAAAAGAACTAACAC | |
| | | AAAAGAAAACTCACAAGGCAAATATAGAGATTAGCCTTGATGAATTTAAGTTCA | |
| | | TGTTAATGCTTGAAAATAACTACCATGAGTTTAAAAGGCTTAACCAATGGGTTT | |
| | | TGAAACCAATAAGTAAAGATTTAAACACTTACAGCAATATGAAATTGGTGGTTG | |
| | | ATAAGCGAGGCCGCCCGACTGATACGTTGATTTTCCAAGTTGAACTAGATAGAC | |
| | | AAATGGATCTCGTAACCGAACTTGAGAACAACCAGATAAAAATGAATGGTGACA | |
| | | AAATACCAACAACCATTACATCAGATTCCTACCTACATAACGGACTAAGAAAAA | |
| | | CACTACACGATGCTTTAACTGCAAAAATTCAGCTCACCAGTTTTGAGGCAAAAT | |
| | | TTTTGAGTGACATGCAAAGTAAGTATGATCTCAATGGTTCGTTCTCATGGCTCA | |
| | | CGCAAAAACAACGAACCACACTAGAGAACATACTGGCTAAATACGGAAGGATCT | |
| | | GAGGTTCTTATGGCTCTTGTATCTATCAGTGAAGCATCAAGACTAACAAACAAA | |
| | | AGTAGAACAACTGTTCACCGTTACATATCAAAGGGAAAACTGTCCATATGCACA | |
| | | GATGAAAACGGTGTAAAAAAGATAGATACATCAGAGCTTTTACGAGTTTTTGGT | |
| | | GCATTCAAAGCTGTTCACCATGAACAGATCGACAATGTAACAGATGAACAGCAT | |
| | | GTAACACCTAATAGAACAGGTGAAACCAGTAAAACAAAGCAACTAGAACATGAA | |
| | | ATTGAACACCTGAGACAACTTGTTACAGCTCAACAGTCACACATAGACAGCCTG | |
| | | AAACAGGCGATGCTGCTTATCGAATCAAAGCTGCCGACAACACGGGAGCCAGTG | |
| | | ACGCCTCCCGTGGGGAAAAAATCATGGCAATTCTGGAAGAAATAGCGCTTTCAG | |
| | | CCGGCAAACCGGCTGAAGCCGGATCTGCGATTCTGATAACAAACTAGCAACACC | |
| | | AGAACAGCCCGTTTGCGGGCAGCAAAACCCGTACCGATTATCAAAAAGGATCTT | |
| | | CACCTAGATCCTTTTAAATTAAAAATGAAGTTTTAAATCAATCTAAAGTATATA | |
| | | TGAGTAAACTTGGTCTGATTACGCCCCGCCCTGCCACTCATCACAGTACTGTTG | |
| | | TAATTCATTAAGCATGCGGCCGACATGGAAGCCATCACAAACGGCATGATGAAC | |
| | | CTGGATCGCCAGCGGCATCAGCACCTTGTCGCCTTGCGTATAATATTTGCCCAT | |
| | | CGTGAAAACGGGGGCGAAGAAGTTGTCCATATTGGCCACGTTTAAATCAAAACT | |
| | | GGTGAAACTCACCCAGGGATTGGCTGAGACAAAAAACATATTCTCAATAAACCC | |
| | | TTTAGGGAAATAGGCCAGGTTTTCACCGTAACACGCCACATCTTGCGAATATAT | |
| | | GTGGAGAAACTGCCGGAAATCGTCGTGGTATTCACTCCAGAGGGACGAAAACGT | |
| | | TTCAGTTTGCTCATGGAAAACGGTGTAACAAGGGTGAACACTATCCCAGATCAC | |
| | | CAGCTCACCGTCTTTCATGGCCATACGAAACTCCGGGTGAGCGTTCATCAGGCG | |
| | | GGCAAGAATGTGAATAAAGGCCGGATAAAACTTGTGCTTATTTTTCTTTACGGT | |
| | | CTTTAAAAAGGCCGTAATATCCAGCTGAACGGTCTGGTTATAGGTACATTGAGC | |
| | | AACTGACTGAAATGCCTCAAAATGTTCTTTACGATGCCATTGGGATATATCAAC | |
| | | GGTGGTATATCCCGTGATTTTTTTCTCCATACTCTTCCTTTTTCAATATTATTG | |
| | | AAGCATTTATCAGGGTTATTGTCTCATGAGCGGATACATATTTGAATGTATTTA | |
| | | GAAAAATAAACAAATAGGGGTTCCGCG | |
| bEG_S2 | N-term | CACATTTCCCCGAAAAGTGCCACCTGACGTCTAAGAAACCATTATTATCATGAC | 585 |
| | HIS₆-Tag | ATTAACCTATAAAAATAGGCGTATCACGAGGCAGAATTTCAGATAAAAAAAATC | |
| | with ATag- | CTTAGCTTTCGCTAAGGATGATTTCTGGAATTCGCGGCCGCTTCTAGAGGGAGA | |
| | 1 | ACGATCGTTGGCTGaatcataaaaaatttatttgctttgtgagcggataacaat | |
| | | 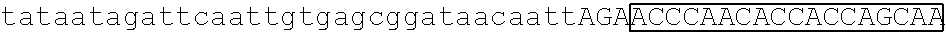 | |
| | | 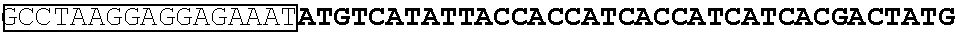 | |
| | | ATATTCCCACAAGCGAGAACTTGTACTTTCAAGGGATGAGCAAAGGAGAAGAAC | |
| | | TTTTCACTGGAGTTGTCCCAATTCTTGTTGAATTAGATGGTGATGTTAATGGGC | |
| | | ACAAATTTTCTGTCCGTGGAGAGGGTGAAGGTGATGCTACAAACGGAAAACTCA | |
| | | CCCTTAAATTTATTTGCACTACTGGAAAACTACCTGTTCCGTGGCCAACACTTG | |
| | | TCACTACTCTGACCTATGGTGTTCAATGCTTTTCCCGTTATCCGGATCACATGA | |
| | | AACGGCATGACTTTTTCAAGAGTGCCATGCCCGAAGGTTATGTACAGGAACGCA | |
| | | CTATATCTTTCAAAGATGACGGGACCTACAAGACGCGTGCTGAAGTCAAGTTTG | |
| | | AAGGTGATACCCTTGTTAATCGTATCGAGTTAAAGGGTATTGATTTTAAAGAAG | |
| | | ATGGAAACATTCTTGGACACAAACTCGAGTACAACTTTAACTCACACAATGTAT | |
| | | ACATCACGGCAGACAAACAAAAGAATGGAATCAAAGCTAACTTCAAAATTCGCC | |
| | | ACAACGTTGAAGATGGTTCCGTTCAACTAGCAGACCATTATCAACAAAATACTC | |
| | | CAATTGGCGATGGCCCTGTCCTTTTACCAGACAACCATTACCTGTCGACACAAT | |
| | | CTGTCCTTTCGAAAGATCCCAACGAAAAGCGTGACCACATGGTCCTTCTTGAGT | |
| | | TTGTAACTGCTGCTGGGATTACACATGGCATGGATGAGCTCTACAAATAATTCA | |
| | | GCCAAAAAACTTAAGACCGCCGGTCTTGTCCACTACCTTGCAGTAATGCGGTGG | |
| | | ACAGGATCGGCGGTTTTCTTTTCTCTTCTCAACCAATGgcggcgcgccatcgaa | |
| | | 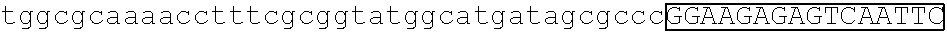 | |
| | | 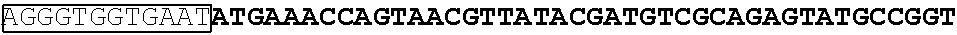 | |
| | | GTCTCTTATCAGACCGTTTCCCGCGTGGTGAACCAGGCCAGCCACGTTTCTGCG | |
| | | AAAACGCGGGAAAAAGTGGAAGCGGCGATGGCGGAGCTGAATTACATTCCCAAC | |
| | | CGCGTGGCACAACAACTGGCGGGCAAACAGTCGTTGCTTATTGGCGTTGCCACC | |
| | | TCCAGTCTGGCCCTGCACGCGCCGTCGCAAATTGTCGCGGCGATTAAATCTCGC | |
| | | GCCGATCAACTGGGTGCCAGCGTGGTGGTGTCGATGGTAGAACGAAGCGGCGTC | |
| | | GAAGCCTGTAAAGCGGCGGTGCACAATCTTCTCGCGCAACGCGTCAGTGGGCTG | |
| | | ATCATTAACTATCCGCTGGATGACCAGGATGCCATTGCTGTGGAAGCTGCCTGC | |
| | | ACTAATGTTCCGGCGTTATTTCTTGATGTCTCTGACCAGACACCCATCAACAGT | |
| | | ATTATTTTCTCCCATGAGGACGGTACGCGACTGGGCGTGGAGCATCTGGTCGCA | |
| | | TTGGGTCACCAGCAAATCGCGCTGTTAGCGGGCCCATTAAGTTCTGTCTCGGCG | |
| | | CGTCTGCGTCTGGCTGGCTGGCATAAATATCTCACTCGCAATCAAATTCAGCCG | |
| | | ATAGCGGAACGGGAAGGCGACTGGAGTGCCATGTCCGGTTTTCAACAAACCATG | |
| | | CAAATGCTGAATGAGGGCATCGTTCCCACTGCGATGCTGGTTGCCAACGATCAG | |
| | | ATGGCGCTGGGCGCAATGCGCGCCATTACCGAGTCCGGGCTGCGCGTTGGTGCG | |
| | | GATATCTCGGTAGTGGGATACGACGATACCGAAGATAGCTCATGTTATATCCCG | |
| | | CCGTTAACCACCATCAAACAGGATTTTCGCCTGCTGGGGCAAACCAGCGTGGAC | |
| | | CGCTTGCTGCAACTCTCTCAGGGCCAGGCGGTGAAGGGCAATCAGCTGTTGCCA | |
| | | GTCTCACTGGTGAAAAGAAAAACCACCCTGGCGCCCAATACGCAAACCGCCTCT | |
| | | CCCCGCGCGTTGGCCGATTCATTAATGCAGCTGGCACGACAGGTTTCCCGACTG | |
| | | GAAAGCGGGCAGTGATAATTGGTAACGAATCAGACAATTGACGGCTCGAGGGAG | |
| | | TAGCATAGGGTTTGCAGAATCCCTGCTTCGTCCATTTGACAGGCACATTATGCA | |
| | | TCGATGATAAGCTGTCAAACATGAGCACGCTTACTAGTAGCGGCCGCTGCAGTC | |
| | | CGGCAAAAAAGGGCAAGGTGTCACCACCCTGCCCTTTTTCTTTAAAACCGAAAA | |
| | | GATTACTTCGCGTTATGCAGGCTTCCTCGCTCACTGACTCGCTGCGCTCGGTCG | |
| | | TTCGGCTGCGGCGAGCGGTATCAGCTCACTCAAAGGCGGTAATGACAGTAAGAC | |
| | | GGGTAAGCCTGTTGATGATACCGCTGCCTTACTGGGTGCATTAGCCAGTCTGAA | |
| | | TGACCTGTCACGGGATAATCCGAAGTGGTCAGACTGGAAAATCAGAGGGCAGGA | |
| | | ACTGCTGAACAGCAAAAAGTCAGATAGCACCACATAGCAGACCCGCCATAAAAC | |
| | | GCCCTGAGAAGCCCGTGACGGGCTTTTCTTGTATTATGGGTAGTTTCCTTGCAT | |
| | | GAATCCATAAAAGGCGCCTGTAGTGCCATTTACCCCCATTCACTGCCAGAGCCG | |
| | | TGAGCGCAGCGAACTGAATGTCACGAAAAAGACAGCGACTCAGGTGCCTGATGG | |
| | | TCGGAGACAAAAGGAATATTCAGCGATTTGCCCGAGCTTGCGAGGGTGCTACTT | |
| | | AAGCCTTTAGGGTTTTAAGGTCTGTTTTGTAGAGGAGCAAACAGCGTTTGCGAC | |
| | | ATCCTTTTGTAATACTGCGGAACTGACTAAAGTAGTGAGTTATACACAGGGCTG | |
| | | GGATCTATTCTTTTTATCTTTTTTTATTCTTTCTTTATTCTATAAATTATAACC | |
| | | ACTTGAATATAAACAAAAAAAACACACAAAGGTCTAGCGGAATTTACAGAGGGT | |
| | | CTAGCAGAATTTACAAGTTTTCCAGCAAAGGTCTAGCAGAATTTACAGATACCC | |
| | | ACAACTCAAAGGAAAAGGACTAGTAATTATCATTGACTAGCCCATCTCAATTGG | |
| | | TATAGTGATTAAAATCACCTAGACCAATTGAGATGTATGTCTGAATTAGTTGTT | |
| | | TTCAAAGCAAATGAACTAGCGATTAGTCGCTATGACTTAACGGAGCATGAAACC | |
| | | AAGCTAATTTTATGCTGTGTGGCACTACTCAACCCCACGATTGAAAACCCTACA | |
| | | AGGAAAGAACGGACGGTATCGTTCACTTATAACCAATACGCTCAGATGATGAAC | |
| | | ATCAGTAGGGAAAATGCTTATGGTGTATTAGCTAAAGCAACCAGAGAGCTGATG | |
| | | ACGAGAACTGTGGAAATCAGGAATCCTTTGGTTAAAGGCTTTTGGATTTTCCAG | |
| | | TGGACAAACTATGCCAAGTTCTCAAGCGAAAAATTAGAATTAGTTTTTAGTGAA | |
| | | GAGATATTGCCTTATCTTTTCCAGTTAAAAAAATTCATAAAATATAATCTGGAA | |
| | | CATGTTAAGTCTTTTGAAAACAAATACTCTATGAGGATTTATGAGTGGTTATTA | |
| | | AAAGAACTAACACAAAAGAAAACTCACAAGGCAAATATAGAGATTAGCCTTGAT | |
| | | GAATTTAAGTTCATGTTAATGCTTGAAAATAACTACCATGAGTTTAAAAGGCTT | |
| | | AACCAATGGGTTTTGAAACCAATAAGTAAAGATTTAAACACTTACAGCAATATG | |
| | | AAATTGGTGGTTGATAAGCGAGGCCGCCCGACTGATACGTTGATTTTCCAAGTT | |
| | | GAACTAGATAGACAAATGGATCTCGTAACCGAACTTGAGAACAACCAGATAAAA | |
| | | ATGAATGGTGACAAAATACCAACAACCATTACATCAGATTCCTACCTACATAAC | |
| | | GGACTAAGAAAAACACTACACGATGCTTTAACTGCAAAAATTCAGCTCACCAGT | |
| | | TTTGAGGCAAAATTTTTGAGTGACATGCAAAGTAAGTATGATCTCAATGGTTCG | |
| | | TTCTCATGGCTCACGCAAAAACAACGAACCACACTAGAGAACATACTGGCTAAA | |
| | | TACGGAAGGATCTGAGGTTCTTATGGCTCTTGTATCTATCAGTGAAGCATCAAG | |
| | | ACTAACAAACAAAAGTAGAACAACTGTTCACCGTTACATATCAAAGGGAAAACT | |
| | | GTCCATATGCACAGATGAAAACGGTGTAAAAAAGATAGATACATCAGAGCTTTT | |
| | | ACGAGTTTTTGGTGCATTCAAAGCTGTTCACCATGAACAGATCGACAATGTAAC | |
| | | AGATGAACAGCATGTAACACCTAATAGAACAGGTGAAACCAGTAAAACAAAGCA | |
| | | ACTAGAACATGAAATTGAACACCTGAGACAACTTGTTACAGCTCAACAGTCACA | |
| | | CATAGACAGCCTGAAACAGGCGATGCTGCTTATCGAATCAAAGCTGCCGACAAC | |
| | | ACGGGAGCCAGTGACGCCTCCCGTGGGGAAAAAATCATGGCAATTCTGGAAGAA | |
| | | ATAGCGCTTTCAGCCGGCAAACCGGCTGAAGCCGGATCTGCGATTCTGATAACA | |
| | | AACTAGCAACACCAGAACAGCCCGTTTGCGGGCAGCAAAACCCGTACCGATTAT | |
| | | CAAAAAGGATCTTCACCTAGATCCTTTTAAATTAAAAATGAAGTTTTAAATCAA | |
| | | TCTAAAGTATATATGAGTAAACTTGGTCTGACAGTTACCAATGCTTAATCAGTG | |
| | | AGGCACCTATCTCAGCGATCTGTCTATTTCGTTCATCCATAGTTGCCTGACTCC | |
| | | CCGTCGTGTAGATAACTACGATACGGGAGGGCTTACCATCTGGCCCCAGTGCTG | |
| | | CAATGATACCGCGAGAACCACGCTCACCGGCTCCAGATTTATCAGCAATAAACC | |
| | | AGCCAGCCGGAAGGGCCGAGCGCAGAAGTGGTCCTGCAACTTTATCCGCCTCCA | |
| | | TCCAGTCTATTAATTGTTGCCGGGAAGCTAGAGTAAGTAGTTCGCCAGTTAATA | |
| | | GTTTGCGCAACGTTGTTGCCATTGCTACAGGCATCGTGGTGTCACGCTCGTCGT | |
| | | TTGGTATGGCTTCATTCAGCTCCGGTTCCCAACGATCAAGGCGAGTTACATGAT | |
| | | CCCCCATGTTGTGCAAAAAAGCGGTTAGCTCCTTCGGTCCTCCGATCGTTGTCA | |
| | | GAAGTAAGTTGGCCGCAGTGTTATCACTCATGGTTATGGCAGCACTGCATAATT | |
| | | CTCTTACTGTCATGCCATCCGTAAGATGCTTTTCTGTGACTGGTGAGTACTCAA | |
| | | CCAAGTCATTCTGAGAATAGTGTATGCGGCGACCGAGTTGCTCTTGCCCGGCGT | |
| | | CAATACGGGATAATACCGCGCCACATAGCAGAACTTTAAAAGTGCTCATCATTG | |
| | | GAAAACGTTCTTCGGGGCGAAAACTCTCAAGGATCTTACCGCTGTTGAGATCCA | |
| | | GTTCGATATAACCCACTCGTGCACCCAACTGATCTTCAGCATCTTTTACTTTCA | |
| | | CCAGCGTTTCTGGGTGAGCAAAAACAGGAAGGCAAAATGCCGCAAAAAAGGGAA | |
| | | TAAGGGCGACACGGAAATGTTGAATACTCATACTCTTCCTTTTTCAATATTATT | |
| | | GAAGCATTTATCAGGGTTATTGTCTCATGAGCGGATACATATTTGAATGTATTT | |
| | | AGAAAAATAAACAAATAGGGGTTCCGCG | |
| pEG3045 | HIS₆-MdnA | ATGGCATATCCCAACGATCAACAAGGTAAAGCACTTCCTTTCTTTGCTCGTTTC | 586 |
| | | TTGTCCGTAAGCAAAGAGGAATCTTCCATCAAGTCTCCTTCCCCTGAGCCTACC | |
| | | TACGGGGGCACCTTTAAATACCCTTCTGACTGGGAAGATTATTAATAA | |
| pEG3046 | HIS₆-BmbC | ATGGGTCCGGTTGTTGTGTTCGATTGCATGACGGCCGACTTTCTGAACGACGAT | 587 |
| | | CCAAATAACGCGGAGTTGTCTGCCTTGGAAATGGAGGAGCTCGAGTCCTGGGGC | |
| | | GCCTGGGACGGAGAGGCTACCAGCTAGTAA | |
| pEG3047 | HIS₆-StrA | ATGAGTAAGGAATTAGAAAAAGTTCTTGAATCCAGTTCAATGGCAAAGGGGGAC | 588 |
| | | GGCTGGAAGGTTATGGCTAAAGGTGACGGTTGGGAGTAATAA | |
| pEG3048 | HIS₆-PqqA | ATGTGGAAGAAACCTGCTTTTATCGATTTACGTCTCGGTCTGGAAGTGACGCTG | 589 |
| | | TACATTTCTAACCGTTAATAA | |
| pEG3049 | HIS₆-SboA | ATGAAAAAAGCTGTCATTGTAGAAAACAAAGGTTGTGCAACATGCTCGATCGGA | 590 |
| | | GCCGCTTGTCTAGTGGACGGTCCTATCCCTGATTTTGAAATTGCCGGTGCAACA | |
| | | GGTCTATTCGGTCTATGGGGATAA | |
| pEG3051 | HIS₆-TfxA | ATGGATAACAAGGTTGCGAAGAATGTCGAAGTGAAGAAGGGCTCCATCAAGGCG | 591 |
| | | ACCTTCAAGGCTGCTGTTCTGAAGTCGAAGACGAAGGTCGACATCGGAGGTAGC | |
| | | CGTCAGGGCTGCGTCGCTTAATAA | |
| pEG3052 | HIS₆- | ATGTCAGAAGAACAACTCAAGGCATTCATTGCCAAGGTTCAAGCAGACACTTCA | 592 |
| | ProcA1.7 | CTGCAGGAACAGCTCAAAGTAGAAGGTGCTGATGTTGTTGCTATTGCTAAAGCC | |
| | | TCAGGGTTCGCGATTACCACAGAGGACTTAAAAGCACATCAAGCCAACTCACAA | |
| | | AAGAACCTGTCTGATGCTGAGCTGGAAGGTGTGGCTGGGCGAACCATTGGGGGA | |
| | | ACCATTGTGTCGATAACCTGTGAGACTTGCGATCTGCTTGTGGGGAAAATGTGC | |
| | | TGATAA | |
| PEG3053 | HIS₆-TbtA | ATGGACCTGAATGATCTGCCGATGGATGTTTTTGAACTGGCAGATAGCGGTGTT | 593 |
| | | GCAGTTGAAAGCCTGACCGCAGGTCATGGTATGACCGAAGTTGGTGCAAGCTGT | |
| | | AATTGCTTTTGTTATATTTGTTGTAGCTGCAGCAGCGCCTAATAA | |
| pEG3055 | HIS₆-Pgm2 | ATGGAGCGCGAAATCGTGTGGACAGAAATTGAGGAGTCGGATTTAGCCGCCGTC | 594 |
| | | GTGTCGGCATCTAATGTCAAGGATGGTCCAACCGTTAGCTCAAGTAATGTAAAG | |
| | | GACCGCTAATAA | |
| bEG_S3 | RST_{N}* | CACATTTCCCCGAAAAGTGCCACCTGACGTCTAAGAAACCATTATTATCATGAC | 595 |
| | expression | ATTAACCTATAAAAATAGGCGTATCACGAGGCAGAATTTCAGATAAAAAAAATC | |
| | vector | CTTAGCTTTCGCTAAGGATGATTTCTGGAATTCGCGGCCGCTTCTAGAGGGAGA | |
| | | ACGATCGTTGGCTGaatcataaaaaatttatttgctttgtgagcggataacaat | |
| | | 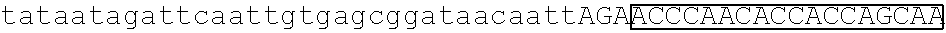 | |
| | | 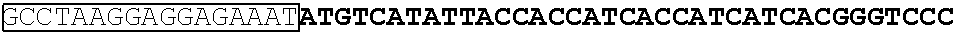 | |
| | | TGCAGGACTCAGAAGTCAATCAAGAAGCTAAGCCAGAGGTCAAGCCAGAAGTCA | |
| | | AGCCTGAGACTCACATCAATTTAAAGGTGTCCGATGGATCTTCAGAGATCTTCT | |
| | | TCAAGATCAAAAAGACCACTCCTTTAAGAAGGCTGATGGAAGCGTTCGCTAAAA | |
| | | GACAGGGTAAGGAAATGGACTCCTTAAGATTCTTGTACGACGGTATTAGAATTC | |
| | | AAGCTGATCAGGCCCCTGAAGATTTGGACATGGAGGATAACGATATTATTGAGG | |
| | | CTCACCGCGAACAGATTGGAGGTCATCACCATCACCACCATGGATATGATATTA | |
| | | GCACAGGTATGAGCAAAGGAGAAGAACTTTTCACTGGAGTTGTCCCAATTCTTG | |
| | | TTGAATTAGATGGTGATGTTAATGGGCACAAATTTTCTGTCCGTGGAGAGGGTG | |
| | | AAGGTGATGCTACAAACGGAAAACTCACCCTTAAATTTATTTGCACTACTGGAA | |
| | | AACTACCTGTTCCGTGGCCAACACTTGTCACTACTCTGACCTATGGTGTTCAAT | |
| | | GCTTTTCCCGTTATCCGGATCACATGAAACGGCATGACTTTTTCAAGAGTGCCA | |
| | | TGCCCGAAGGTTATGTACAGGAACGCACTATATCTTTCAAAGATGACGGGACCT | |
| | | ACAAGACGCGTGCTGAAGTCAAGTTTGAAGGTGATACCCTTGTTAATCGTATCG | |
| | | AGTTAAAGGGTATTGATTTTAAAGAAGATGGAAACATTCTTGGACACAAACTCG | |
| | | AGTACAACTTTAACTCACACAATGTATACATCACGGCAGACAAACAAAAGAATG | |
| | | GAATCAAAGCTAACTTCAAAATTCGCCACAACGTTGAAGATGGTTCCGTTCAAC | |
| | | TAGCAGACCATTATCAACAAAATACTCCAATTGGCGATGGCCCTGTCCTTTTAC | |
| | | CAGACAACCATTACCTGTCGACACAATCTGTCCTTTCGAAAGATCCCAACGAAA | |
| | | AGCGTGACCACATGGTCCTTCTTGAGTTTGTAACTGCTGCTGGGATTACACATG | |
| | | GCATGGATGAGCTCTACAAATAATTCAGCCAAAAAACTTAAGACCGCCGGTCTT | |
| | | GTCCACTACCTTGCAGTAATGCGGTGGACAGGATCGGCGGTTTTCTTTTCTCTT | |
| | | CTCAACCAATGgcggcgcgccatcgaatggcgcaaaacctttcgcggtatggca | |
| | | 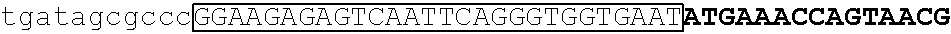 | |
| | | TTATACGATGTCGCAGAGTATGCCGGTGTCTCTTATCAGACCGTTTCCCGCGTG | |
| | | GTGAACCAGGCCAGCCACGTTTCTGCGAAAACGCGGGAAAAAGTGGAAGCGGCG | |
| | | ATGGCGGAGCTGAATTACATTCCCAACCGCGTGGCACAACAACTGGCGGGCAAA | |
| | | CAGTCGTTGCTTATTGGCGTTGCCACCTCCAGTCTGGCCCTGCACGCGCCGTCG | |
| | | CAAATTGTCGCGGCGATTAAATCTCGCGCCGATCAACTGGGTGCCAGCGTGGTG | |
| | | GTGTCGATGGTAGAACGAAGCGGCGTCGAAGCCTGTAAAGCGGCGGTGCACAAT | |
| | | CTTCTCGCGCAACGCGTCAGTGGGCTGATCATTAACTATCCGCTGGATGACCAG | |
| | | GATGCCATTGCTGTGGAAGCTGCCTGCACTAATGTTCCGGCGTTATTTCTTGAT | |
| | | GTCTCTGACCAGACACCCATCAACAGTATTATTTTCTCCCATGAGGACGGTACG | |
| | | CGACTGGGCGTGGAGCATCTGGTCGCATTGGGTCACCAGCAAATCGCGCTGTTA | |
| | | GCGGGCCCATTAAGTTCTGTCTCGGCGCGTCTGCGTCTGGCTGGCTGGCATAAA | |
| | | TATCTCACTCGCAATCAAATTCAGCCGATAGCGGAACGGGAAGGCGACTGGAGT | |
| | | GCCATGTCCGGTTTTCAACAAACCATGCAAATGCTGAATGAGGGCATCGTTCCC | |
| | | ACTGCGATGCTGGTTGCCAACGATCAGATGGCGCTGGGCGCAATGCGCGCCATT | |
| | | ACCGAGTCCGGGCTGCGCGTTGGTGCGGATATCTCGGTAGTGGGATACGACGAT | |
| | | ACCGAAGATAGCTCATGTTATATCCCGCCGTTAACCACCATCAAACAGGATTTT | |
| | | CGCCTGCTGGGGCAAACCAGCGTGGACCGCTTGCTGCAACTCTCTCAGGGCCAG | |
| | | GCGGTGAAGGGCAATCAGCTGTTGCCAGTCTCACTGGTGAAAAGAAAAACCACC | |
| | | CTGGCGCCCAATACGCAAACCGCCTCTCCCCGCGCGTTGGCCGATTCATTAATG | |
| | | CAGCTGGCACGACAGGTTTCCCGACTGGAAAGCGGGCAGTGATAATTGGTAACG | |
| | | AATCAGACAATTGACGGCTCGAGGGAGTAGCATAGGGTTTGCAGAATCCCTGCT | |
| | | TCGTCCATTTGACAGGCACATTATGCATCGATGATAAGCTGTCAAACATGAGCA | |
| | | CGCTTACTAGTAGCGGCCGCTGCAGTCCGGCAAAAAAGGGCAAGGTGTCACCAC | |
| | | CCTGCCCTTTTTCTTTAAAACCGAAAAGATTACTTCGCGTTATGCAGGCTTCCT | |
| | | CGCTCACTGACTCGCTGCGCTCGGTCGTTCGGCTGCGGCGAGCGGTATCAGCTC | |
| | | ACTCAAAGGCGGTAATGACAGTAAGACGGGTAAGCCTGTTGATGATACCGCTGC | |
| | | CTTACTGGGTGCATTAGCCAGTCTGAATGACCTGTCACGGGATAATCCGAAGTG | |
| | | GTCAGACTGGAAAATCAGAGGGCAGGAACTGCTGAACAGCAAAAAGTCAGATAG | |
| | | CACCACATAGCAGACCCGCCATAAAACGCCCTGAGAAGCCCGTGACGGGCTTTT | |
| | | CTTGTATTATGGGTAGTTTCCTTGCATGAATCCATAAAAGGCGCCTGTAGTGCC | |
| | | ATTTACCCCCATTCACTGCCAGAGCCGTGAGCGCAGCGAACTGAATGTCACGAA | |
| | | AAAGACAGCGACTCAGGTGCCTGATGGTCGGAGACAAAAGGAATATTCAGCGAT | |
| | | TTGCCCGAGCTTGCGAGGGTGCTACTTAAGCCTTTAGGGTTTTAAGGTCTGTTT | |
| | | TGTAGAGGAGCAAACAGCGTTTGCGACATCCTTTTGTAATACTGCGGAACTGAC | |
| | | TAAAGTAGTGAGTTATACACAGGGCTGGGATCTATTCTTTTTATCTTTTTTTAT | |
| | | TCTTTCTTTATTCTATAAATTATAACCACTTGAATATAAACAAAAAAAACACAC | |
| | | AAAGGTCTAGCGGAATTTACAGAGGGTCTAGCAGAATTTACAAGTTTTCCAGCA | |
| | | AAGGTCTAGCAGAATTTACAGATACCCACAACTCAAAGGAAAAGGACTAGTAAT | |
| | | TATCATTGACTAGCCCATCTCAATTGGTATAGTGATTAAAATCACCTAGACCAA | |
| | | TTGAGATGTATGTCTGAATTAGTTGTTTTCAAAGCAAATGAACTAGCGATTAGT | |
| | | CGCTATGACTTAACGGAGCATGAAACCAAGCTAATTTTATGCTGTGTGGCACTA | |
| | | CTCAACCCCACGATTGAAAACCCTACAAGGAAAGAACGGACGGTATCGTTCACT | |
| | | TATAACCAATACGCTCAGATGATGAACATCAGTAGGGAAAATGCTTATGGTGTA | |
| | | TTAGCTAAAGCAACCAGAGAGCTGATGACGAGAACTGTGGAAATCAGGAATCCT | |
| | | TTGGTTAAAGGCTTTTGGATTTTCCAGTGGACAAACTATGCCAAGTTCTCAAGC | |
| | | GAAAAATTAGAATTAGTTTTTAGTGAAGAGATATTGCCTTATCTTTTCCAGTTA | |
| | | AAAAAATTCATAAAATATAATCTGGAACATGTTAAGTCTTTTGAAAACAAATAC | |
| | | TCTATGAGGATTTATGAGTGGTTATTAAAAGAACTAACACAAAAGAAAACTCAC | |
| | | AAGGCAAATATAGAGATTAGCCTTGATGAATTTAAGTTCATGTTAATGCTTGAA | |
| | | AATAACTACCATGAGTTTAAAAGGCTTAACCAATGGGTTTTGAAACCAATAAGT | |
| | | AAAGATTTAAACACTTACAGCAATATGAAATTGGTGGTTGATAAGCGAGGCCGC | |
| | | CCGACTGATACGTTGATTTTCCAAGTTGAACTAGATAGACAAATGGATCTCGTA | |
| | | ACCGAACTTGAGAACAACCAGATAAAAATGAATGGTGACAAAATACCAACAACC | |
| | | ATTACATCAGATTCCTACCTACATAACGGACTAAGAAAAACACTACACGATGCT | |
| | | TTAACTGCAAAAATTCAGCTCACCAGTTTTGAGGCAAAATTTTTGAGTGACATG | |
| | | CAAAGTAAGTATGATCTCAATGGTTCGTTCTCATGGCTCACGCAAAAACAACGA | |
| | | ACCACACTAGAGAACATACTGGCTAAATACGGAAGGATCTGAGGTTCTTATGGC | |
| | | TCTTGTATCTATCAGTGAAGCATCAAGACTAACAAACAAAAGTAGAACAACTGT | |
| | | TCACCGTTACATATCAAAGGGAAAACTGTCCATATGCACAGATGAAAACGGTGT | |
| | | AAAAAAGATAGATACATCAGAGCTTTTACGAGTTTTTGGTGCATTCAAAGCTGT | |
| | | TCACCATGAACAGATCGACAATGTAACAGATGAACAGCATGTAACACCTAATAG | |
| | | AACAGGTGAAACCAGTAAAACAAAGCAACTAGAACATGAAATTGAACACCTGAG | |
| | | ACAACTTGTTACAGCTCAACAGTCACACATAGACAGCCTGAAACAGGCGATGCT | |
| | | GCTTATCGAATCAAAGCTGCCGACAACACGGGAGCCAGTGACGCCTCCCGTGGG | |
| | | GAAAAAATCATGGCAATTCTGGAAGAAATAGCGCTTTCAGCCGGCAAACCGGCT | |
| | | GAAGCCGGATCTGCGATTCTGATAACAAACTAGCAACACCAGAACAGCCCGTTT | |
| | | GCGGGCAGCAAAACCCGTACCGATTATCAAAAAGGATCTTCACCTAGATCCTTT | |
| | | TAAATTAAAAATGAAGTTTTAAATCAATCTAAAGTATATATGAGTAAACTTGGT | |
| | | CTGACAGTTACCAATGCTTAATCAGTGAGGCACCTATCTCAGCGATCTGTCTAT | |
| | | TTCGTTCATCCATAGTTGCCTGACTCCCCGTCGTGTAGATAACTACGATACGGG | |
| | | AGGGCTTACCATCTGGCCCCAGTGCTGCAATGATACCGCGAGAACCACGCTCAC | |
| | | CGGCTCCAGATTTATCAGCAATAAACCAGCCAGCCGGAAGGGCCGAGCGCAGAA | |
| | | GTGGTCCTGCAACTTTATCCGCCTCCATCCAGTCTATTAATTGTTGCCGGGAAG | |
| | | CTAGAGTAAGTAGTTCGCCAGTTAATAGTTTGCGCAACGTTGTTGCCATTGCTA | |
| | | CAGGCATCGTGGTGTCACGCTCGTCGTTTGGTATGGCTTCATTCAGCTCCGGTT | |
| | | CCCAACGATCAAGGCGAGTTACATGATCCCCCATGTTGTGCAAAAAAGCGGTTA | |
| | | GCTCCTTCGGTCCTCCGATCGTTGTCAGAAGTAAGTTGGCCGCAGTGTTATCAC | |
| | | TCATGGTTATGGCAGCACTGCATAATTCTCTTACTGTCATGCCATCCGTAAGAT | |
| | | GCTTTTCTGTGACTGGTGAGTACTCAACCAAGTCATTCTGAGAATAGTGTATGC | |
| | | GGCGACCGAGTTGCTCTTGCCCGGCGTCAATACGGGATAATACCGCGCCACATA | |
| | | GCAGAACTTTAAAAGTGCTCATCATTGGAAAACGTTCTTCGGGGCGAAAACTCT | |
| | | CAAGGATCTTACCGCTGTTGAGATCCAGTTCGATATAACCCACTCGTGCACCCA | |
| | | ACTGATCTTCAGCATCTTTTACTTTCACCAGCGTTTCTGGGTGAGCAAAAACAG | |
| | | GAAGGCAAAATGCCGCAAAAAAGGGAATAAGGGCGACACGGAAATGTTGAATAC | |
| | | TCATACTCTTCCTTTTTCAATATTATTGAAGCATTTATCAGGGTTATTGTCTCA | |
| | | TGAGCGGATACATATTTGAATGTATTTAGAAAAATAAACAAATAGGGGTTCCGC | |
| | | G | |
| pEG3057 | RSTx* | ATGAACAAGAAGAACATTTTACCGCAGTTAGGACAACCAGTCATCCGCCTTACT | 596 |
| | TruE* | GCCGGTCAACTGTCAAGCCAACTGGCGGAGCTTTCTGAGGAGGCTCTGGGAGGG | |
| | | GTCGATGCCTCGTACGCGGTGTTCTGGCCGATCTGTAGCTATGACGACTAATAA | |
| pEG3058 | RSTx*- | ATGGCATATCCCAACGATCAACAAGGTAAAGCACTTCCTTTCTTTGCTCGTTTC | 597 |
| | MdnA | TTGTCCGTAAGCAAAGAGGAATCTTCCATCAAGTCTCCTTCCCCTGAGCCTACC | |
| | | TACGGGGGCACCTTTAAATACCCTTCTGACTGGGAAGATTATTAATAA | |
| pEG3059 | RSTx*- | ATGAAAAAAGCTGTCATTGTAGAAAACAAAGGTTGTGCAACATGCTCGATCGGA | 598 |
| | SboA | GCCGCTTGTCTAGTGGACGGTCCTATCCCTGATTTTGAAATTGCCGGTGCAACA | |
| | | GGTCTATTCGGTCTATGGGGATAA | |
| pEG3060 | RSTx* | ATGTGGAAGAAACCTGCTTTTATCGATTTACGTCTCGGTCTGGAAGTGACGCTG | 599 |
| | PqqA | TACATTTCTAACCGTTAATAA | |
| pEG3061 | RST_{N}*-StrA | ATGAGTAAGGAATTAGAAAAAGTTCTTGAATCCAGTTCAATGGCAAAGGGGGAC | 600 |
| | | GGCTGGAAGGTTATGGCTAAAGGTGACGGTTGGGAGTAATAA | |
| pEG3062 | RST_{N}*- | ATGGGTCCGGTTGTTGTGTTCGATTGCATGACGGCCGACTTTCTGAACGACGAT | 601 |
| | BmbC | CCAAATAACGCGGAGTTGTCTGCCTTGGAAATGGAGGAGCTCGAGTCCTGGGGC | |
| | | GCCTGGGACGGAGAGGCTACCAGCTAGTAA | |
| pEG3063 | RST_{N}*- | ATGGATAACAAGGTTGCGAAGAATGTCGAAGTGAAGAAGGGCTCCATCAAGGCG | 602 |
| | TfxA | ACCTTCAAGGCTGCTGTTCTGAAGTCGAAGACGAAGGTCGACATCGGAGGTAGC | |
| | | CGTCAGGGCTGCGTCGCTTAATAA | |
| pEG3064 | RST_{N}*- | ATGTCAGAAGAACAACTCAAGGCATTCATTGCCAAGGTTCAAGCAGACACTTCA | 603 |
| | ProcA1.7 | CTGCAGGAACAGCTCAAAGTAGAAGGTGCTGATGTTGTTGCTATTGCTAAAGCC | |
| | | TCAGGGTTCGCGATTACCACAGAGGACTTAAAAGCACATCAAGCCAACTCACAA | |
| | | AAGAACCTGTCTGATGCTGAGCTGGAAGGTGTGGCTGGGCGAACCATTGGGGGA | |
| | | ACCATTGTGTCGATAACCTGTGAGACTTGCGATCTGCTTGTGGGGAAAATGTGC | |
| | | TGATAA | |
| pEG3065 | RST_{N}*- | ATGGACCTGAATGATCTGCCGATGGATGTTTTTGAACTGGCAGATAGCGGTGTT | 604 |
| | TbtA | GCAGTTGAAAGCCTGACCGCAGGTCATGGTATGACCGAAGTTGGTGCAAGCTGT | |
| | | AATTGCTTTTGTTATATTTGTTGTAGCTGCAGCAGCGCCTAATAA | |
| pEG3067 | RST_{N}*- | ATGGAGCGCGAAATCGTGTGGACAGAAATTGAGGAGTCGGATTTAGCCGCCGTC | 605 |
| | Pgm2 | GTGTCGGCATCTAATGTCAAGGATGGTCCAACCGTTAGCTCAAGTAATGTAAAG | |
| | | GACCGCTAATAA | |
| bEG_S4 | RST_{N} | CACATTTCCCCGAAAAGTGCCACCTGACGTCTAAGAAACCATTATTATCATGAC | 606 |
| | expression | ATTAACCTATAAAAATAGGCGTATCACGAGGCAGAATTTCAGATAAAAAAAATC | |
| | vector | CTTAGCTTTCGCTAAGGATGATTTCTGGAATTCGCGGCCGCTTCTAGAGGGAGA | |
| | | ACGATCGTTGGCTGaatcataaaaaatttatttgctttgtgagcggataacaat | |
| | | 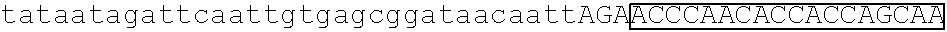 | |
| | | 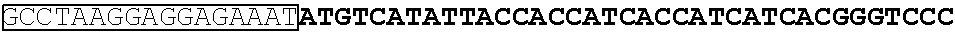 | |
| | | TGCAGGACTCAGAAGTCAATCAAGAAGCTAAGCCAGAGGTCAAGCCAGAAGTCA | |
| | | AGCCTGAGACTCACATCAATTTAAAGGTGTCCGATGGATCTTCAGAGATCTTCT | |
| | | TCAAGATCAAAAAGACCACTCCTTTAAGAAGGCTGATGGAAGCGTTCGCTAAAA | |
| | | GACAGGGTAAGGAAATGGACTCCTTAAGATTCTTGTACGACGGTATTAGAATTC | |
| | | AAGCTGATCAGGCCCCTGAAGATTTGGACATGGAGGATAACGATATTATTGAGG | |
| | | CTCACCGCGAACAGATTGGAGGTTGCATGTCATATTACGACTCCATTCCCACAA | |
| | | GCGAGAACTTGTACTTTCAAGGGTGCATGAGCAAAGGAGAAGAACTTTTCACTG | |
| | | GAGTTGTCCCAATTCTTGTTGAATTAGATGGTGATGTTAATGGGCACAAATTTT | |
| | | CTGTCCGTGGAGAGGGTGAAGGTGATGCTACAAACGGAAAACTCACCCTTAAAT | |
| | | TTATTTGCACTACTGGAAAACTACCTGTTCCGTGGCCAACACTTGTCACTACTC | |
| | | TGACCTATGGTGTTCAATGCTTTTCCCGTTATCCGGATCACATGAAACGGCATG | |
| | | ACTTTTTCAAGAGTGCCATGCCCGAAGGTTATGTACAGGAACGCACTATATCTT | |
| | | TCAAAGATGACGGGACCTACAAGACGCGTGCTGAAGTCAAGTTTGAAGGTGATA | |
| | | CCCTTGTTAATCGTATCGAGTTAAAGGGTATTGATTTTAAAGAAGATGGAAACA | |
| | | TTCTTGGACACAAACTCGAGTACAACTTTAACTCACACAATGTATACATCACGG | |
| | | CAGACAAACAAAAGAATGGAATCAAAGCTAACTTCAAAATTCGCCACAACGTTG | |
| | | AAGATGGTTCCGTTCAACTAGCAGACCATTATCAACAAAATACTCCAATTGGCG | |
| | | ATGGCCCTGTCCTTTTACCAGACAACCATTACCTGTCGACACAATCTGTCCTTT | |
| | | CGAAAGATCCCAACGAAAAGCGTGACCACATGGTCCTTCTTGAGTTTGTAACTG | |
| | | CTGCTGGGATTACACATGGCATGGATGAGCTCTACAAATAATTCAGCCAAAAAA | |
| | | CTTAAGACCGCCGGTCTTGTCCACTACCTTGCAGTAATGCGGTGGACAGGATCG | |
| | | GCGGTTTTCTTTTCTCTTCTCAACCAATGgcggcgcgccatcgaatggcgcaaa | |
| | | acctttcgcggtatggcatgatagcgcccGGAAGAGAGTCAATTCAGGGTGGTG | |
| | | AATATGAAACCAGTAACGTTATACGATGTCGCAGAGTATGCCGGTGTCTCTTAT | |
| | | CAGACCGTTTCCCGCGTGGTGAACCAGGCCAGCCACGTTTCTGCGAAAACGCGG | |
| | | GAAAAAGTGGAAGCGGCGATGGCGGAGCTGAATTACATTCCCAACCGCGTGGCA | |
| | | CAACAACTGGCGGGCAAACAGTCGTTGCTTATTGGCGTTGCCACCTCCAGTCTG | |
| | | GCCCTGCACGCGCCGTCGCAAATTGTCGCGGCGATTAAATCTCGCGCCGATCAA | |
| | | CTGGGTGCCAGCGTGGTGGTGTCGATGGTAGAACGAAGCGGCGTCGAAGCCTGT | |
| | | AAAGCGGCGGTGCACAATCTTCTCGCGCAACGCGTCAGTGGGCTGATCATTAAC | |
| | | TATCCGCTGGATGACCAGGATGCCATTGCTGTGGAAGCTGCCTGCACTAATGTT | |
| | | CCGGCGTTATTTCTTGATGTCTCTGACCAGACACCCATCAACAGTATTATTTTC | |
| | | TCCCATGAGGACGGTACGCGACTGGGCGTGGAGCATCTGGTCGCATTGGGTCAC | |
| | | CAGCAAATCGCGCTGTTAGCGGGCCCATTAAGTTCTGTCTCGGCGCGTCTGCGT | |
| | | CTGGCTGGCTGGCATAAATATCTCACTCGCAATCAAATTCAGCCGATAGCGGAA | |
| | | CGGGAAGGCGACTGGAGTGCCATGTCCGGTTTTCAACAAACCATGCAAATGCTG | |
| | | AATGAGGGCATCGTTCCCACTGCGATGCTGGTTGCCAACGATCAGATGGCGCTG | |
| | | GGCGCAATGCGCGCCATTACCGAGTCCGGGCTGCGCGTTGGTGCGGATATCTCG | |
| | | GTAGTGGGATACGACGATACCGAAGATAGCTCATGTTATATCCCGCCGTTAACC | |
| | | ACCATCAAACAGGATTTTCGCCTGCTGGGGCAAACCAGCGTGGACCGCTTGCTG | |
| | | CAACTCTCTCAGGGCCAGGCGGTGAAGGGCAATCAGCTGTTGCCAGTCTCACTG | |
| | | GTGAAAAGAAAAACCACCCTGGCGCCCAATACGCAAACCGCCTCTCCCCGCGCG | |
| | | TTGGCCGATTCATTAATGCAGCTGGCACGACAGGTTTCCCGACTGGAAAGCGGG | |
| | | CAGTGATAATTGGTAACGAATCAGACAATTGACGGCTCGAGGGAGTAGCATAGG | |
| | | GTTTGCAGAATCCCTGCTTCGTCCATTTGACAGGCACATTATGCATCGATGATA | |
| | | AGCTGTCAAACATGAGCACGCTTACTAGTAGCGGCCGCTGCAGTCCGGCAAAAA | |
| | | AGGGCAAGGTGTCACCACCCTGCCCTTTTTCTTTAAAACCGAAAAGATTACTTC | |
| | | GCGTTATGCAGGCTTCCTCGCTCACTGACTCGCTGCGCTCGGTCGTTCGGCTGC | |
| | | GGCGAGCGGTATCAGCTCACTCAAAGGCGGTAATGACAGTAAGACGGGTAAGCC | |
| | | TGTTGATGATACCGCTGCCTTACTGGGTGCATTAGCCAGTCTGAATGACCTGTC | |
| | | ACGGGATAATCCGAAGTGGTCAGACTGGAAAATCAGAGGGCAGGAACTGCTGAA | |
| | | CAGCAAAAAGTCAGATAGCACCACATAGCAGACCCGCCATAAAACGCCCTGAGA | |
| | | AGCCCGTGACGGGCTTTTCTTGTATTATGGGTAGTTTCCTTGCATGAATCCATA | |
| | | AAAGGCGCCTGTAGTGCCATTTACCCCCATTCACTGCCAGAGCCGTGAGCGCAG | |
| | | CGAACTGAATGTCACGAAAAAGACAGCGACTCAGGTGCCTGATGGTCGGAGACA | |
| | | AAAGGAATATTCAGCGATTTGCCCGAGCTTGCGAGGGTGCTACTTAAGCCTTTA | |
| | | GGGTTTTAAGGTCTGTTTTGTAGAGGAGCAAACAGCGTTTGCGACATCCTTTTG | |
| | | TAATACTGCGGAACTGACTAAAGTAGTGAGTTATACACAGGGCTGGGATCTATT | |
| | | CTTTTTATCTTTTTTTATTCTTTCTTTATTCTATAAATTATAACCACTTGAATA | |
| | | TAAACAAAAAAAACACACAAAGGTCTAGCGGAATTTACAGAGGGTCTAGCAGAA | |
| | | TTTACAAGTTTTCCAGCAAAGGTCTAGCAGAATTTACAGATACCCACAACTCAA | |
| | | AGGAAAAGGACTAGTAATTATCATTGACTAGCCCATCTCAATTGGTATAGTGAT | |
| | | TAAAATCACCTAGACCAATTGAGATGTATGTCTGAATTAGTTGTTTTCAAAGCA | |
| | | AATGAACTAGCGATTAGTCGCTATGACTTAACGGAGCATGAAACCAAGCTAATT | |
| | | TTATGCTGTGTGGCACTACTCAACCCCACGATTGAAAACCCTACAAGGAAAGAA | |
| | | CGGACGGTATCGTTCACTTATAACCAATACGCTCAGATGATGAACATCAGTAGG | |
| | | GAAAATGCTTATGGTGTATTAGCTAAAGCAACCAGAGAGCTGATGACGAGAACT | |
| | | GTGGAAATCAGGAATCCTTTGGTTAAAGGCTTTTGGATTTTCCAGTGGACAAAC | |
| | | TATGCCAAGTTCTCAAGCGAAAAATTAGAATTAGTTTTTAGTGAAGAGATATTG | |
| | | CCTTATCTTTTCCAGTTAAAAAAATTCATAAAATATAATCTGGAACATGTTAAG | |
| | | TCTTTTGAAAACAAATACTCTATGAGGATTTATGAGTGGTTATTAAAAGAACTA | |
| | | ACACAAAAGAAAACTCACAAGGCAAATATAGAGATTAGCCTTGATGAATTTAAG | |
| | | TTCATGTTAATGCTTGAAAATAACTACCATGAGTTTAAAAGGCTTAACCAATGG | |
| | | GTTTTGAAACCAATAAGTAAAGATTTAAACACTTACAGCAATATGAAATTGGTG | |
| | | GTTGATAAGCGAGGCCGCCCGACTGATACGTTGATTTTCCAAGTTGAACTAGAT | |
| | | AGACAAATGGATCTCGTAACCGAACTTGAGAACAACCAGATAAAAATGAATGGT | |
| | | GACAAAATACCAACAACCATTACATCAGATTCCTACCTACATAACGGACTAAGA | |
| | | AAAACACTACACGATGCTTTAACTGCAAAAATTCAGCTCACCAGTTTTGAGGCA | |
| | | AAATTTTTGAGTGACATGCAAAGTAAGTATGATCTCAATGGTTCGTTCTCATGG | |
| | | CTCACGCAAAAACAACGAACCACACTAGAGAACATACTGGCTAAATACGGAAGG | |
| | | ATCTGAGGTTCTTATGGCTCTTGTATCTATCAGTGAAGCATCAAGACTAACAAA | |
| | | CAAAAGTAGAACAACTGTTCACCGTTACATATCAAAGGGAAAACTGTCCATATG | |
| | | CACAGATGAAAACGGTGTAAAAAAGATAGATACATCAGAGCTTTTACGAGTTTT | |
| | | TGGTGCATTCAAAGCTGTTCACCATGAACAGATCGACAATGTAACAGATGAACA | |
| | | GCATGTAACACCTAATAGAACAGGTGAAACCAGTAAAACAAAGCAACTAGAACA | |
| | | TGAAATTGAACACCTGAGACAACTTGTTACAGCTCAACAGTCACACATAGACAG | |
| | | CCTGAAACAGGCGATGCTGCTTATCGAATCAAAGCTGCCGACAACACGGGAGCC | |
| | | AGTGACGCCTCCCGTGGGGAAAAAATCATGGCAATTCTGGAAGAAATAGCGCTT | |
| | | TCAGCCGGCAAACCGGCTGAAGCCGGATCTGCGATTCTGATAACAAACTAGCAA | |
| | | CACCAGAACAGCCCGTTTGCGGGCAGCAAAACCCGTACCGATTATCAAAAAGGA | |
| | | TCTTCACCTAGATCCTTTTAAATTAAAAATGAAGTTTTAAATCAATCTAAAGTA | |
| | | TATATGAGTAAACTTGGTCTGACAGTTACCAATGCTTAATCAGTGAGGCACCTA | |
| | | TCTCAGCGATCTGTCTATTTCGTTCATCCATAGTTGCCTGACTCCCCGTCGTGT | |
| | | AGATAACTACGATACGGGAGGGCTTACCATCTGGCCCCAGTGCTGCAATGATAC | |
| | | CGCGAGAACCACGCTCACCGGCTCCAGATTTATCAGCAATAAACCAGCCAGCCG | |
| | | GAAGGGCCGAGCGCAGAAGTGGTCCTGCAACTTTATCCGCCTCCATCCAGTCTA | |
| | | TTAATTGTTGCCGGGAAGCTAGAGTAAGTAGTTCGCCAGTTAATAGTTTGCGCA | |
| | | ACGTTGTTGCCATTGCTACAGGCATCGTGGTGTCACGCTCGTCGTTTGGTATGG | |
| | | CTTCATTCAGCTCCGGTTCCCAACGATCAAGGCGAGTTACATGATCCCCCATGT | |
| | | TGTGCAAAAAAGCGGTTAGCTCCTTCGGTCCTCCGATCGTTGTCAGAAGTAAGT | |
| | | TGGCCGCAGTGTTATCACTCATGGTTATGGCAGCACTGCATAATTCTCTTACTG | |
| | | TCATGCCATCCGTAAGATGCTTTTCTGTGACTGGTGAGTACTCAACCAAGTCAT | |
| | | TCTGAGAATAGTGTATGCGGCGACCGAGTTGCTCTTGCCCGGCGTCAATACGGG | |
| | | ATAATACCGCGCCACATAGCAGAACTTTAAAAGTGCTCATCATTGGAAAACGTT | |
| | | CTTCGGGGCGAAAACTCTCAAGGATCTTACCGCTGTTGAGATCCAGTTCGATAT | |
| | | AACCCACTCGTGCACCCAACTGATCTTCAGCATCTTTTACTTTCACCAGCGTTT | |
| | | CTGGGTGAGCAAAAACAGGAAGGCAAAATGCCGCAAAAAAGGGAATAAGGGCGA | |
| | | CACGGAAATGTTGAATACTCATACTCTTCCTTTTTCAATATTATTGAAGCATTT | |
| | | ATCAGGGTTATTGTCTCATGAGCGGATACATATTTGAATGTATTTAGAAAAATA | |
| | | AACAAATAGGGGTTCCGCG | |
| pEG3121 | MdnA* | ATGGCACTTCCTTTCTTTGCTCGTTTCTTGTCCGTAAGCAAAGAGGAATCTTCC | 607 |
| | | ATCAAGTCTCCTTCCCCTGAGCCTACCTACGGGGGCACCTTTAAATACCCTTCT | |
| | | GACTGGGAAGATTATTAATAA | |
| pEG3128 | ProcA* | ATGTCAGAAGAACAACTCAAGGCATTCATTGCCAAGGTTCAAGCAGACACTTCA | 608 |
| | | CTGCAGGAACAGCTCAAAGTAGAAGGTGCTGATGTTGTTGCTATTGCTAAAGCC | |
| | | TCAGGGTTCGCGATTACCACAGAGGACCTCAATTCGCATCGCCAAAATCTGTCT | |
| | | GATGATGAGCTGGAGGGAGTCGCGGGAGGCTTTTTCTGCGTACAGGGTACGGCC | |
| | | AACCGTTTCACTATCAACGTTTGCTGATAA | |
| pEG3132 | PaaP | ATGATTAAATTTTCTACATTGTCTCAGCGCATCAGCGCCATCACGGAAGAAAAC | 609 |
| | | GCCATGTACACTAAGGGTCAAGTGATCGTATTGAGCTGATAA | |
| pEG3248 | SboA | ATGAAAAAAGCTGTCATTGTAGAAAACAAAGGTTGTGCAACATGCTCGATCGGA | 610 |
| | | GCCGCTTGTCTAGTGGACGGTCCTATCCCTGATTTTGAAATTGCCGGTGCAACA | |
| | | GGTCTATTCGGTCTATGGGGATAA | |
| bEG_S5 | RSTn | CACATTTCCCCGAAAAGTGCCACCTGACGTCTAAGAAACCATTATTATCATGAC | 611 |
| | expression | ATTAACCTATAAAAATAGGCGTATCACGAGGCAGAATTTCAGATAAAAAAAATC | |
| | vector (w/ | CTTAGCTTTCGCTAAGGATGATTTCTGGAATTCGCGGCCGCTTCTAGAGGGAGG | |
| | flanking | GTCTCAGTGCAACGATCGTTGGCTGaatcataaaaaatttatttgctttgtgag | |
| | restriction | 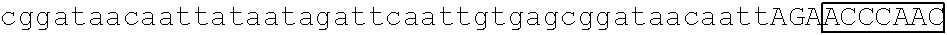 | |
| | sites) | 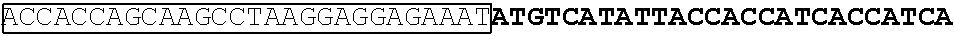 | |
| | | TCACGGGTCCCTGCAGGACTCAGAAGTCAATCAAGAAGCTAAGCCAGAGGTCAA | |
| | | GCCAGAAGTCAAGCCTGAGACTCACATCAATTTAAAGGTGTCCGATGGATCTTC | |
| | | AGAGATCTTCTTCAAGATCAAAAAGACCACTCCTTTAAGAAGGCTGATGGAAGC | |
| | | GTTCGCTAAAAGACAGGGTAAGGAAATGGACTCCTTAAGATTCTTGTACGACGG | |
| | | TATTAGAATTCAAGCTGATCAGGCCCCTGAAGATTTGGACATGGAGGATAACGA | |
| | | TATTATTGAGGCTCACCGCGAACAGATTGGAGGTTGCATGTCATATTACGACTC | |
| | | CATTCCCACAAGCGAGAACTTGTACTTTCAAGGGTGCATGAGCAAAGGAGAAGA | |
| | | ACTTTTCACTGGAGTTGTCCCAATTCTTGTTGAATTAGATGGTGATGTTAATGG | |
| | | GCACAAATTTTCTGTCCGTGGAGAGGGTGAAGGTGATGCTACAAACGGAAAACT | |
| | | CACCCTTAAATTTATTTGCACTACTGGAAAACTACCTGTTCCGTGGCCAACACT | |
| | | TGTCACTACTCTGACCTATGGTGTTCAATGCTTTTCCCGTTATCCGGATCACAT | |
| | | GAAACGGCATGACTTTTTCAAGAGTGCCATGCCCGAAGGTTATGTACAGGAACG | |
| | | CACTATATCTTTCAAAGATGACGGGACCTACAAGACGCGTGCTGAAGTCAAGTT | |
| | | TGAAGGTGATACCCTTGTTAATCGTATCGAGTTAAAGGGTATTGATTTTAAAGA | |
| | | AGATGGAAACATTCTTGGACACAAACTCGAGTACAACTTTAACTCACACAATGT | |
| | | ATACATCACGGCAGACAAACAAAAGAATGGAATCAAAGCTAACTTCAAAATTCG | |
| | | CCACAACGTTGAAGATGGTTCCGTTCAACTAGCAGACCATTATCAACAAAATAC | |
| | | TCCAATTGGCGATGGCCCTGTCCTTTTACCAGACAACCATTACCTGTCGACACA | |
| | | ATCTGTCCTTTCGAAAGATCCCAACGAAAAGCGTGACCACATGGTCCTTCTTGA | |
| | | GTTTGTAACTGCTGCTGGGATTACACATGGCATGGATGAGCTCTACAAATAATT | |
| | | CAGCCAAAAAACTTAAGACCGCCGGTCTTGTCCACTACCTTGCAGTAATGCGGT | |
| | | GGACAGGATCGGCGGTTTTCTTTTCTCTTCTCAACAAGTGAGACCATGGgcggc | |
| | | 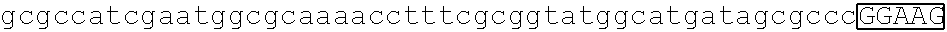 | |
| | | 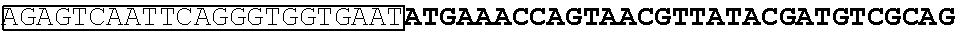 | |
| | | AGTATGCCGGTGTCTCTTATCAGACCGTTTCCCGCGTGGTGAACCAGGCCAGCC | |
| | | ACGTTTCTGCGAAAACGCGGGAAAAAGTGGAAGCGGCGATGGCGGAGCTGAATT | |
| | | ACATTCCCAACCGCGTGGCACAACAACTGGCGGGCAAACAGTCGTTGCTTATTG | |
| | | GCGTTGCCACCTCCAGTCTGGCCCTGCACGCGCCGTCGCAAATTGTCGCGGCGA | |
| | | TTAAATCTCGCGCCGATCAACTGGGTGCCAGCGTGGTGGTGTCGATGGTAGAAC | |
| | | GAAGCGGCGTCGAAGCCTGTAAAGCGGCGGTGCACAATCTTCTCGCGCAACGCG | |
| | | TCAGTGGGCTGATCATTAACTATCCGCTGGATGACCAGGATGCCATTGCTGTGG | |
| | | AAGCTGCCTGCACTAATGTTCCGGCGTTATTTCTTGATGTCTCTGACCAGACAC | |
| | | CCATCAACAGTATTATTTTCTCCCATGAGGACGGTACGCGACTGGGCGTGGAGC | |
| | | ATCTGGTCGCATTGGGTCACCAGCAAATCGCGCTGTTAGCGGGCCCATTAAGTT | |
| | | CTGTCTCGGCGCGTCTGCGTCTGGCTGGCTGGCATAAATATCTCACTCGCAATC | |
| | | AAATTCAGCCGATAGCGGAACGGGAAGGCGACTGGAGTGCCATGTCCGGTTTTC | |
| | | AACAAACCATGCAAATGCTGAATGAGGGCATCGTTCCCACTGCGATGCTGGTTG | |
| | | CCAACGATCAGATGGCGCTGGGCGCAATGCGCGCCATTACCGAGTCCGGGCTGC | |
| | | GCGTTGGTGCGGATATCTCGGTAGTGGGATACGACGATACCGAAGATAGCTCAT | |
| | | GTTATATCCCGCCGTTAACCACCATCAAACAGGATTTTCGCCTGCTGGGGCAAA | |
| | | CCAGCGTGGACCGCTTGCTGCAACTCTCTCAGGGCCAGGCGGTGAAGGGCAATC | |
| | | AGCTGTTGCCAGTCTCACTGGTGAAAAGAAAAACCACCCTGGCGCCCAATACGC | |
| | | AAACCGCCTCTCCCCGCGCGTTGGCCGATTCATTAATGCAGCTGGCACGACAGG | |
| | | TTTCCCGACTGGAAAGCGGGCAGTGATAATTGGTAACGAATCAGACAATTGACG | |
| | | GCTCGAGGGAGTAGCATAGGGTTTGCAGAATCCCTGCTTCGTCCATTTGACAGG | |
| | | CACATTATGCATCGATGATAAGCTGTCAAACATGAGCACGCTTACTAGTAGCGG | |
| | | CCGCTGCAGTCCGGCAAAAAAGGGCAAGGTGTCACCACCCTGCCCTTTTTCTTT | |
| | | AAAACCGAAAAGATTACTTCGCGTTATGCAGGCTTCCTCGCTCACTGACTCGCT | |
| | | GCGCTCGGTCGTTCGGCTGCGGCGAGCGGTATCAGCTCACTCAAAGGCGGTAAT | |
| | | GACAGTAAGACGGGTAAGCCTGTTGATGATACCGCTGCCTTACTGGGTGCATTA | |
| | | GCCAGTCTGAATGACCTGTCACGGGATAATCCGAAGTGGTCAGACTGGAAAATC | |
| | | AGAGGGCAGGAACTGCTGAACAGCAAAAAGTCAGATAGCACCACATAGCAGACC | |
| | | CGCCATAAAACGCCCTGAGAAGCCCGTGACGGGCTTTTCTTGTATTATGGGTAG | |
| | | TTTCCTTGCATGAATCCATAAAAGGCGCCTGTAGTGCCATTTACCCCCATTCAC | |
| | | TGCCAGAGCCGTGAGCGCAGCGAACTGAATGTCACGAAAAAGACAGCGACTCAG | |
| | | GTGCCTGATGGTCGGAGACAAAAGGAATATTCAGCGATTTGCCCGAGCTTGCGA | |
| | | GGGTGCTACTTAAGCCTTTAGGGTTTTAAGGTCTGTTTTGTAGAGGAGCAAACA | |
| | | GCGTTTGCGACATCCTTTTGTAATACTGCGGAACTGACTAAAGTAGTGAGTTAT | |
| | | ACACAGGGCTGGGATCTATTCTTTTTATCTTTTTTTATTCTTTCTTTATTCTAT | |
| | | AAATTATAACCACTTGAATATAAACAAAAAAAACACACAAAGGTCTAGCGGAAT | |
| | | TTACAGAGGGTCTAGCAGAATTTACAAGTTTTCCAGCAAAGGTCTAGCAGAATT | |
| | | TACAGATACCCACAACTCAAAGGAAAAGGACTAGTAATTATCATTGACTAGCCC | |
| | | ATCTCAATTGGTATAGTGATTAAAATCACCTAGACCAATTGAGATGTATGTCTG | |
| | | AATTAGTTGTTTTCAAAGCAAATGAACTAGCGATTAGTCGCTATGACTTAACGG | |
| | | AGCATGAAACCAAGCTAATTTTATGCTGTGTGGCACTACTCAACCCCACGATTG | |
| | | AAAACCCTACAAGGAAAGAACGGACGGTATCGTTCACTTATAACCAATACGCTC | |
| | | AGATGATGAACATCAGTAGGGAAAATGCTTATGGTGTATTAGCTAAAGCAACCA | |
| | | GAGAGCTGATGACGAGAACTGTGGAAATCAGGAATCCTTTGGTTAAAGGCTTTT | |
| | | GGATTTTCCAGTGGACAAACTATGCCAAGTTCTCAAGCGAAAAATTAGAATTAG | |
| | | TTTTTAGTGAAGAGATATTGCCTTATCTTTTCCAGTTAAAAAAATTCATAAAAT | |
| | | ATAATCTGGAACATGTTAAGTCTTTTGAAAACAAATACTCTATGAGGATTTATG | |
| | | AGTGGTTATTAAAAGAACTAACACAAAAGAAAACTCACAAGGCAAATATAGAGA | |
| | | TTAGCCTTGATGAATTTAAGTTCATGTTAATGCTTGAAAATAACTACCATGAGT | |
| | | TTAAAAGGCTTAACCAATGGGTTTTGAAACCAATAAGTAAAGATTTAAACACTT | |
| | | ACAGCAATATGAAATTGGTGGTTGATAAGCGAGGCCGCCCGACTGATACGTTGA | |
| | | TTTTCCAAGTTGAACTAGATAGACAAATGGATCTCGTAACCGAACTTGAGAACA | |
| | | ACCAGATAAAAATGAATGGTGACAAAATACCAACAACCATTACATCAGATTCCT | |
| | | ACCTACATAACGGACTAAGAAAAACACTACACGATGCTTTAACTGCAAAAATTC | |
| | | AGCTCACCAGTTTTGAGGCAAAATTTTTGAGTGACATGCAAAGTAAGTATGATC | |
| | | TCAATGGTTCGTTCTCATGGCTCACGCAAAAACAACGAACCACACTAGAGAACA | |
| | | TACTGGCTAAATACGGAAGGATCTGAGGTTCTTATGGCTCTTGTATCTATCAGT | |
| | | GAAGCATCAAGACTAACAAACAAAAGTAGAACAACTGTTCACCGTTACATATCA | |
| | | AAGGGAAAACTGTCCATATGCACAGATGAAAACGGTGTAAAAAAGATAGATACA | |
| | | TCAGAGCTTTTACGAGTTTTTGGTGCATTCAAAGCTGTTCACCATGAACAGATC | |
| | | GACAATGTAACAGATGAACAGCATGTAACACCTAATAGAACAGGTGAAACCAGT | |
| | | AAAACAAAGCAACTAGAACATGAAATTGAACACCTGAGACAACTTGTTACAGCT | |
| | | CAACAGTCACACATAGACAGCCTGAAACAGGCGATGCTGCTTATCGAATCAAAG | |
| | | CTGCCGACAACACGGGAGCCAGTGACGCCTCCCGTGGGGAAAAAATCATGGCAA | |
| | | TTCTGGAAGAAATAGCGCTTTCAGCCGGCAAACCGGCTGAAGCCGGATCTGCGA | |
| | | TTCTGATAACAAACTAGCAACACCAGAACAGCCCGTTTGCGGGCAGCAAAACCC | |
| | | GTACCGATTATCAAAAAGGATCTTCACCTAGATCCTTTTAAATTAAAAATGAAG | |
| | | TTTTAAATCAATCTAAAGTATATATGAGTAAACTTGGTCTGACAGTTACCAATG | |
| | | CTTAATCAGTGAGGCACCTATCTCAGCGATCTGTCTATTTCGTTCATCCATAGT | |
| | | TGCCTGACTCCCCGTCGTGTAGATAACTACGATACGGGAGGGCTTACCATCTGG | |
| | | CCCCAGTGCTGCAATGATACCGCGAGAACCACGCTCACCGGCTCCAGATTTATC | |
| | | AGCAATAAACCAGCCAGCCGGAAGGGCCGAGCGCAGAAGTGGTCCTGCAACTTT | |
| | | ATCCGCCTCCATCCAGTCTATTAATTGTTGCCGGGAAGCTAGAGTAAGTAGTTC | |
| | | GCCAGTTAATAGTTTGCGCAACGTTGTTGCCATTGCTACAGGCATCGTGGTGTC | |
| | | ACGCTCGTCGTTTGGTATGGCTTCATTCAGCTCCGGTTCCCAACGATCAAGGCG | |
| | | AGTTACATGATCCCCCATGTTGTGCAAAAAAGCGGTTAGCTCCTTCGGTCCTCC | |
| | | GATCGTTGTCAGAAGTAAGTTGGCCGCAGTGTTATCACTCATGGTTATGGCAGC | |
| | | ACTGCATAATTCTCTTACTGTCATGCCATCCGTAAGATGCTTTTCTGTGACTGG | |
| | | TGAGTACTCAACCAAGTCATTCTGAGAATAGTGTATGCGGCGACCGAGTTGCTC | |
| | | TTGCCCGGCGTCAATACGGGATAATACCGCGCCACATAGCAGAACTTTAAAAGT | |
| | | GCTCATCATTGGAAAACGTTCTTCGGGGCGAAAACTCTCAAGGATCTTACCGCT | |
| | | GTTGAGATCCAGTTCGATATAACCCACTCGTGCACCCAACTGATCTTCAGCATC | |
| | | TTTTACTTTCACCAGCGTTTCTGGGTGAGCAAAAACAGGAAGGCAAAATGCCGC | |
| | | AAAAAAGGGAATAAGGGCGACACGGAAATGTTGAATACTCATACTCTTCCTTTT | |
| | | TCAATATTATTGAAGCATTTATCAGGGTTATTGTCTCATGAGCGGATACATATT | |
| | | TGAATGTATTTAGAAAAATAAACAAATAGGGGTTCCGCG | |
| pEG2192 | PapoA | AGCAAGAAAGAATGGCAAGAGCCCACGATCGAAGTGCTCGATATTAATCAGACT | 612 |
| | | ATGGCGGGTAAGGGCTGGAAACAGATAGACTGGGTGAGCGACCATGATGCTGAC | |
| | | TTACACAATCCGTCTTAATAA | |
| pEG2194 | BamA | CTGAAAATCCGCAAGGTGAAAATTGTCAGAGCGCAGAACGGCCACTACACGAAC | 613 |
| | | TAATAA | |
| pEG2195 | EpiA | GAAGCAGTTAAAGAGAAGAACGATCTGTTCAACCTGGATGTTAAAGTCAACGCA | 614 |
| | | AAAGAAAGTAACGATAGTGGCGCAGAACCACGCATAGCGTCGAAATTTATTTGC | |
| | | ACACCAGGCTGCGCGAAAACGGGTTCGTTTAACAGCTATTGTTGTTAATAA | |
| pEG2199 | HalA1 | ACGAACTTGCTGAAAGAATGGAAAATGCCCCTGGAACGTACGCATAATAACTCC | 615 |
| | | AACCCGGCGGGAGACATTTTTCAGGAACTGGAAGATCAAGACATACTCGCCGGT | |
| | | GTGAATGGAGCAGAAAACTTATACTTTCAGGGTTGTGCGTGGTATAACATTAGC | |
| | | TGCCGTCTGGGCAACAAAGGAGCCTACTGCACCCTTACAGTTGAGTGCATGCCC | |
| | | TCCTGTAACTGATAA | |
| pEG2200 | HalA2 | GTGAATTCCAAAGACCTGAGAAATCCAGAATTTCGCAAAGCTCAGGGTCTGCAG | 616 |
| | | TTTGTAGATGAAGTTAATGAGAAGGAACTCTCGAGTTTAGCCGGCAGCGAGAAT | |
| | | CTTTACTTTCAAGGCACGACGTGGCCATGTGCGACCGTCGGCGTTTCAGTTGCC | |
| | | TTGTGCCCGACGACCAAATGCACTTCACAGTGCTGATAA | |
| pEG2312 | PapA_tev | TTGAAACAGATCAATGTGATTGCTGGCGTAAAAGAGCCTATTCGCGCCTATGAG | 617 |
| | | AACTTGTATTTCCAGGGTTGTTCGGCTAATGACGCATGCTATTTTTGCGACACG | |
| | | CGTGACAACTGCAAAGCCTGTGATGCCAGTGATTTTTGTATCAAAAGTGATACG | |
| pEG2571 | TruE_tev | AACAAGAAGAACATTTTACCGCAGTTAGGACAACCAGTCATCCGCCTTACTGCC | 618 |
| | | GGTCAACTGTCAAGCCAACTGGCGGAGCTTTCTGAGGAGGCTCTGGGAGAGAAC | |
| | | TTGTATTTCCAGGGTGTCGATGCCTCGACCTTGCCGGTTCCGACGTTGTGTAGC | |
| | | TATGACGGGGTGGACGCTAGCACAGTCCCTACACTTTGTAGTTACGATGAC | |
| pEG2575 | PsnA2_tev | ATGAGCAAAAATGAGAACAACAAGAAACAGCTGCGCGATCTTTTCATTGAAGAT | 619 |
| | | CTGGGCAAAGTTACTGGCGAGAACTTGTATTTCCAGGGTAAAGGTGGCCCGTAT | |
| | | ACCACCTTAGCCATTGGCGAAGAAGATCCGATTACCACTTTGGCTATCGGAGAA | |
| | | GAGGACCCTGATCCAACGACACTTGCCTTAGGTGAAGAGGACCCAACTACGCTT | |
| | | GCAATCGGCGAAGAA | |
| pEG3157 | MibA | ATGCCAGCCGATATTCTGGAGACTCGTACCAGCGAAACGGAGGACTTACTGGAT | 620 |
| | | CTTGACCTGAGCATCGGTGTAGAAGAAATCACCGCAGGCCCGGCAGTGACTTCT | |
| | | TGGTCACTGTGCACCCCTGGATGCACGAGTCCGGGCGGTGGCTCCAATTGTTCG | |
| | | TTCTGTTGCTAATAA | |
| pEG3161 | PlpA1 | ATGAGCATTGAGAATGCCAAGAGCTTTTATGAACGCGTCAGTACAGATAAGCAG | 621 |
| | | TTCCGCACTCAACTGGAAAATACGGCCAGTGCTGAAGAACGGCAGAAAATCATT | |
| | | CAGGCAGCGGGCTTTGAGTTCACCAATCAGGAGTGGGAAATTGCAAAAGAACAG | |
| | | ATTCTTGCGACAAGTGAAAGTAATAACGGTGAACTGTCCGAGGCCGAACTGACC | |
| | | GCCGTCAGCGGTGGGGTTGACTTAAGCATTTTCGAGCTGCTGGACGAAGAACCT | |
| | | TTATTCCCGATTCGTCCTTTGTACGGCCTGCCTATTTAATAA | |
| pEG3162 | PlpA2 | ATGTCTATTGAGAGTGCAAAGGCTTTCTACCAGCGTATGACGGATGACGCATCT | 622 |
| | | TTTCGTACCCCTTTTGAAGCGGAACTGTCGAAAGAGGAGCGCCAACAATTAATC | |
| | | AAAGATAGCGGATATGACTTTACTGCAGAAGAATGGCAACAGGCTATGACCGAG | |
| | | ATCCAGGCGGCACGCTCAAACGAGGAACTGAATGAGGAAGAACTCGAGGCAATT | |
| | | GCCGGGGGCGCTGTGGCCGCAATGTATGGTGTGGTTTTCCCATGGGACAACGAG | |
| | | TTCCCGTGGCCCCGCTGGGGCGGTTAATAA | |
| pEG3165 | PbtA | ATGAACCTGAACGATTTACCTATGGACGTCTTTGAAATGGCAGACAGCGGTATG | 623 |
| | | GAGGTGGAAAGCCTCACGGCTGGCCATGGCATGCCAGAAGTTGGAGCTAGTTGC | |
| | | AACTGTGTGTGCGGGTTTTGCTGCAGCTGCAGTCCGAGCGCGTAATAA | |
| pEG3172 | LtnA1 | ATGAATAAAAACGAAATCGAAACCCAGCCAGTTACGTGGCTGGAGGAAGTTTCT | 624 |
| | | GATCAGAATTTTGATGAGGATGTCTTTGGTGCGTGTAGCACAAACACCTTCTCG | |
| | | CTGAGCGATTACTGGGGTAACAACGGTGCTTGGTGTACACTCACGCACGAATGT | |
| | | ATGGCATGGTGCAAGTAATAA | |
| pEG3173 | LtnA2 | ATGAAGGAAAAGAATATGAAGAAAAACGACACCATCGAACTTCAGCTTGGAAAA | 625 |
| | | TACCTGGAAGATGATATGATCGAACTGGCTGAAGGGGATGAGTCCCATGGGGGT | |
| | | ACTACCCCGGCTACCCCTGCGATTTCTATCCTCAGCGCGTATATCAGCACCAAT | |
| | | ACCTGCCCGACAACTAAGTGTACACGCGCGTGCTAATAA | |
| pEG3174 | CrnA1 | ATGTCCGAACTGAGTATGGAGAAAGTGGTCGGCGAAACATTTGAGGATCTGAGC | 626 |
| | | ATCGCGGAAATGACGATGGTGCAGGGCAGCGGCGACATTAACGGCGAATTTACT | |
| | | ACCTCGCCGGCATGTGTTTATTCCGTTATGGTTGTATCGAAAGCAAGCAGCGCT | |
| | | AAATGTGCGGCCGGTGCATCGGCAGTCTCGGGAGCCATTCTGAGTGCGATTCGT | |
| | | TGCTAATAA | |
| pEG3175 | CrnA2 | ATGAGCGAATCCAACATGAAGAAGGTTGTTGGCGAAACCTTCGAAGATCTGAGC | 627 |
| | | ATCGCAGAAATGACGAAAGTTCAGGGCTCAGGGGACGTGATGCCGGAATCTACC | |
| | | CCAATTTGTGCCGGCTTCGCAACCTTGATGAGTTCTATCGGTCTTGTTAAAACC | |
| | | ATCAAAGGCAATGTCAAAAGTTTCTCCGTCTTAATTTAATAA | |
| pEG3176 | BsjA2 | ATGACCAATGAAGAGATCATTGTCGCGTGGAAAAACCCTAAAGTCCGTGGCAAA | 628 |
| | | AATATGCCAAGTCACCCGAGCGGCGTGGGATTCCAAGAGCTTTCCATCAACGAG | |
| | | ATGGCCCAAGTGACCGGCGGAGCAGTAGAACAGCGTGCAACACCAACCCTGGCA | |
| | | ACCCCGCTGACCCCGCATACCCCGTACGCAACCTATGTGGTTAGCGGAGGCGTG | |
| | | GTTAGCGCGATTTCTGGTATCTTCAGCAACAATAAAACGTGTCTGGGCTAATAA | |
| pEG3177 | BsjA3 | ATGACCAATGAGGAAATTATCGTTGCGTGGAAAAACCCGAAGGTGCGCGGCAAA | 629 |
| | | AACATGCCTTCCCATCCGTCCGGTGTGGGCTTCCAGGAATTATCTATTAATGAA | |
| | | ATGGCACAGGTGACTGGTGGCGCGGTTGAACAGCGCGCGACGCCGGCAACCCCA | |
| | | GCAACACCATGGCTGATTAAAGCGTCTTATGTGGTGAGTGGGGCGGGAGTTTCT | |
| | | TTTGTCGCAAGCTATATCACTGTAAACTAATAA | |
| pEG3178 | CinA | ATGACGGCGAGTATTCTTCAGTCTGTCGTTGATGCGGACTTTCGTGCGGCCCTG | 630 |
| | | ATTGAAAACCCAGCCGCATTCGGCGCGAGCACCGCAGTTTTGCCGACCCCAGTC | |
| | | GAACAGCAGGATCAGGCATCACTGGATTTTTGGACAAAAGATATTGCTGCCACT | |
| | | GAGGCGTTTGCTTGCAAACAGTCTTGCTCATTTGGGCCGTTCACCTTTGTGTGC | |
| | | GACGGGAATACCAAATAATAA | |
| pEG3180 | LasA | ATGGACAAACGTGTGCGTTACGAAAAACCGAGCCTGGTGAAAGAGGGTACGTTT | 631 |
| | | CGCAAAACTACCGCTGGCCTGCGGCGTCTGTTCGCTGACCAGCTGGTTGGCCGC | |
| | | CGTAACATTTAATAA | |
| pEG3181 | AlbsA | ATGGATTCACTGCTGTCAACAGAAACCGTCATTAGTGATGACGAACTGCTTCCG | 632 |
| | | ATTGAAGTTGGTGGTACCGCGGAATTGACAGAGGGGCAGGGCGGCGGTCAGTCC | |
| | | GAGGATAAACGTCGCGCTTATAACTGCTAATAA | |
| pEG3182 | McbA | ATGGAATTAAAAGCGAGTGAATTTGGTGTAGTTTTGTCCGTTGATGCTCTTAAA | 633 |
| | | TTATCACGCCAGTCTCCATTAGGTGTTGGCATTGGTGGTGGTGGCGGCGGCGGC | |
| | | GGCGGCGGCGGTAGCTGCGGTGGTCAAGGTGGCGGTTGTGGTGGTTGCAGCAAC | |
| | | GGTTGTAGTGGTGGAAACGGTGGCAGCGGCGGAAGTGGTTCACATATCTAATAA | |
| pEG3194 | PsnA2 | ATGAGCAAAAATGAGAACAACAAGAAACAGCTGCGCGATCTTTTCATTGAAGAT | 634 |
| | | CTGGGCAAAGTTACTGGCGGTAAAGGTGGCCCGTATACCACCTTAGCCATTGGC | |
| | | GAAGAAGATCCGATTACCACTTTGGCTATCGGAGAAGAGGACCCTGATCCAACG | |
| | | ACACTTGCCTTAGGTGAAGAGGACCCAACTACGCTTGCAATCGGCGAAGAATAA | |
| | | TAA | |
| pEG3197 | AMdnA | ATGCCGGAAAATCGGCAGGAAGATCTCAACGCTCAGGCTGTACCATTCTTCGCG | 635 |
| | | CGTTTCTTGGAGGGTCAAAACTGCGAGGACCTTACTGATGAGGAATCGGAGGCG | |
| | | GTTAGCGGTGGAAAACGCGGCCAAACCCGTAAATATCCAAGCGACTGCGAAGAT | |
| | | GGGAATGGCGTGACCGGTAAACTGCGCGATGAAGATATTGCAGTGACCTTGAAG | |
| | | TACCCATCCGACAATGAAGATAATGGCGGCGGTGAAATTGTGACTCTGAAGTTT | |
| | | CCAAGTGATGATGATGATCAACCAGTAGGCTAATAA | |
| pEG3283 | PapA | ATGTTGAAACAGATCAATGTGATTGCTGGCGTAAAAGAGCCTATTCGCGCCTAT | 636 |
| | | GGTTGTTCGGCTAATGACGCATGCTATTTTTGCGACACGCGTGACAACTGCAAA | |
| | | GCCTGTGATGCCAGTGATTTTTGTATCAAAAGTGATACGTAATAA | |
| pEG3286 | PcpA | ATGTCGAGTAATATCCTCGAAAAAGTTAAGGAGTTTTTCGTCCGGCTGGTGAAG | 637 |
| | | GATGATGCGTTTCAAAGCCAGCTGCAGAACAACAGTATTGATGAAGTTCGAAAT | |
| | | ATCCTGCAGGAGGCCGGGTACATATTCAGCAAAGAAGAATTCGAAACCGCAACC | |
| | | ATTGAATTGCTGGATTTGAAGGAACGCGATGAATTCCACGAGCTGACAGAAGAG | |
| | | GAGCTTGTCACCGCTGTTGGCGGTGTTACGGGCGGGAGTGGTATATATGGCCCG | |
| | | ATTCAAGCTATGTACGGTGCCGTCGTAGGTGATCCAAAACCGGGTAAGGACTGG | |
| | | GGGTGGCGCTTTCCGAGCCCGCTGCCAAAACCGAGTCCGATTCCGAGTCCGTGG | |
| | | AAACCCCCGGTTGATGTCCAGCCTATGTATGGTGTGGTAGTGTCAAACGATAGT | |
| | | TAATAA | |
| pEG3563 | PadeA | AAAAAGCAATATAGCAAACCTAGCCTGGAGGTTCTGGACGTCCACCAGACCATG | 638 |
| | | GCTGGCCCGGGCACTAGTACGCCAGACGCGTTTCAGCCAGATCCAGATGAAGAT | |
| | | GTTCACTATGATTCGTAATAA | |
| pEG3564 | ThcoA | CGCAAGAAAGAATGGCAGACACCAGAACTGGAAGTACTCGATGTACGCCTCACC | 639 |
| | | GCAGCGGGCCCGGGTAAAGCTAAACCGGATGCTGTGCAGCCAGACGAAGATGAA | |
| | | ATAGTGCACTACTCATAATAA | |
| pEG3565 | StspA | AAGAAATTCTATGAAGCGCCAGCTCTCATCGAACGTGGCGCCTTTGCGGCTGCT | 640 |
| | | ACAGCGGGGTTTGGACGTCTGCTGGCGGATCAGCTGGTGGGACGCCTGATTCCG | |
| | | TAATAA | |
| pEG3567 | LcnA | ACTAAAGGCCTGGACAAAATGCTTTTAACCAAAAAGAAGAAGGATAGTATGGGT | 641 |
| | | CTGCTGAACGAAATCGACGTTACCACCCTGGATGAACAGTTAGGCGGTAAAATG | |
| | | AGCAAAGCATGGTGCCGATCCATGGTGGTGTCCTGCGTGTATAACCTGGTTGAT | |
| | | TTTTCGTCGTCGAGTGACGGGAAAAAGACATGTGCTCTGTACCGCAAATATTGT | |
| | | TAATAA | |
| pEG3568 | PalA | AAAGATCTTCTGAAGGAACTGATGTATGAAGTAGACCTCGAAGAGATGGAGAAT | 642 |
| | | CTTCAGGGTAGCGGGTACTCAGCCGCCCAGTGTGCCTGGATGGCGCTGAGCTGC | |
| | | GTCAATTACATCCCGGGAGTGGGATTCGGTTGTGGCGGCTACAGCGCATGTGAA | |
| | | CTCTACAAGCGTTATTGTTAATAA | |
| pEG3570 | RaxX | AACCACTCTAAGAAAAGTCCGGCAAAAGGGGCAGCGTCCCTGCAGCGTCCTGCT | 643 |
| | | GGGGCAAAAGGCCGCCCTGAACCTCTGGATCAACGCTTGTGGAAACACGTCGGT | |
| | | GGTGGTGACTACCCACCCCCAGGAGCCAACCCAAAGCATGATCCACCACCCCGC | |
| | | AATCCGGGCCACCATTAATAA | |
| pEG3571 | ComX | CAAGATCTGATTAATTACTTCCTGAATTATCCTGAGGCTCTGAAGAAACTCAAG | 644 |
| | | AATAAGGAAGCCTGCTTAATTGGGTTTGACGTCCAGGAAACCGAAACGATTATC | |
| | | AAAGCCTATAACGATTACTACCGCGCTGATCCGATCACGCGTCAATGGGGTGAT | |
| | | TAATAA | |
| pEG3572 | KgpE | AAGAACCCGACGCTGTTGCCCAAACTGACCGCGCCGGTCGAACGTCCGGCCGTA | 645 |
| | | ACTTCGTCGGATTTAAAGCAAGCCTCAAGCGTCGATGCTGCATGGTTAAATGGC | |
| | | GATAATAACTGGTCAACCCCATTCGCCGGTGTGAACGCGGCATGGTTAAATGGG | |
| | | GACAACAACTGGTCCACGCCTTTTGCGGGCGTGAATGCTGCATGGCTTAATGGC | |
| | | GACAATAACTGGAGCACTCCATTTGCCGCCGATGGCGCTGAGTAATAA | |
| pEG3574 | TgnA* | TATCGACCTTATATTGCCAAGTATGTCGAAGAACAAACTCTGCAGAATTCAACC | 646 |
| | | AACCTGGTATATGACGACATCACGCAGATCTCTTTTATCAATAAAGAAAAGAAC | |
| | | GTGAAAAAAATTAATCTGGGTCCCGATACTACGATCGTGACTGAAACCATCGAG | |
| | | AATGCGGACCCCGATGAGTATTTCTTATAATAA | |
| pEG3871 | SgbA | TCTGGTCGCGGGCGCGATCCTGATGCTGCTGTACCTCCCTTGCCTCGTGTACCT | 647 |
| | | CGCACTACTAATCATGAGCCACGTACGGCGTCCCGAGAACCAAGAGCAGCTCCA | |
| | | AGAACTGGACCTACACGTCCGCCTTCGTCGCGTCCATCTCCGTGTGGTCACTCT | |
| | | CCTCAAACCCCTGGTGCAGGACGCAGTGGATGTCGTGTGGAGCGTCAAAAATCG | |
| | | GCTGCGGCTTCGTCTGAGAAGGAAAAGACAATGGAGAACCAAGATTTGGAGTTA | |
| | | TTAGCACGCCTGCATGCACTTCCTGAGACTGAACCGGTGGGCGTCGACGGATTA | |
| | | CCCTATGGCGAGACTTGTGAGTGCGTCGGGTTACTTACGTTGTTGAACACCGTA | |
| | | TGTATCGGCATTTCATGCGCTTAATAA | |
| pEG3905 | TruE | ATGAACAAGAAGAACATTTTACCGCAGTTAGGACAACCAGTCATCCGCCTTACT | 648 |
| | | GCCGGTCAACTGTCAAGCCAACTGGCGGAGCTTTCTGAGGAGGCTCTGGGAGTC | |
| | | GATGCCTCGACCTTGCCGGTTCCGACGTTGTGTAGCTATGACGGGGTGGACGCT | |
| | | AGCACAGTCCCTACACTTTGTAGTTACGATGAC | |
| bEG_S6 | RSTc | CACATTTCCCCGAAAAGTGCCACCTGACGTCTAAGAAACCATTATTATCATGAC | 649 |
| | expression | ATTAACCTATAAAAATAGGCGTATCACGAGGCAGAATTTCAGATAAAAAAAATC | |
| | vector | CTTAGCTTTCGCTAAGGATGATTTCTGGAATTCGCGGCCGCTTCTAGAGGGAGA | |
| | | ACGATCGTTGGCTGaatcataaaaaatttatttgctttgtgagcggataacaat | |
| | | 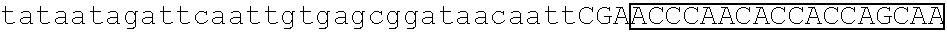 | |
| | | 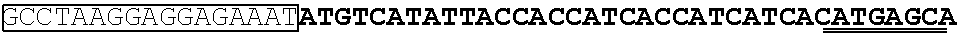 | |
| | | AAGGAGAAGAACTTTTCACTGGAGTTGTCCCAATTCTTGTTGAATTAGATGGTG | |
| | | ATGTTAATGGGCACAAATTTTCTGTCCGTGGAGAGGGTGAAGGTGATGCTACAA | |
| | | ACGGAAAACTCACCCTTAAATTTATTTGCACTACTGGAAAACTACCTGTTCCGT | |
| | | GGCCAACACTTGTCACTACTCTGACCTATGGTGTTCAATGCTTTTCCCGTTATC | |
| | | CGGATCACATGAAACGGCATGACTTTTTCAAGAGTGCCATGCCCGAAGGTTATG | |
| | | TACAGGAACGCACTATATCTTTCAAAGATGACGGGACCTACAAGACGCGTGCTG | |
| | | AAGTCAAGTTTGAAGGTGATACCCTTGTTAATCGTATCGAGTTAAAGGGTATTG | |
| | | ATTTTAAAGAAGATGGAAACATTCTTGGACACAAACTCGAGTACAACTTTAACT | |
| | | CACACAATGTATACATCACGGCAGACAAACAAAAGAATGGAATCAAAGCTAACT | |
| | | TCAAAATTCGCCACAACGTTGAAGATGGTTCCGTTCAACTAGCAGACCATTATC | |
| | | AACAAAATACTCCAATTGGCGATGGCCCTGTCCTTTTACCAGACAACCATTACC | |
| | | TGTCGACACAATCTGTCCTTTCGAAAGATCCCAACGAAAAGCGTGACCACATGG | |
| | | TCCTTCTTGAGTTTGTAACTGCTGCTGGGATTACACATGGCATGGATGAGCTCT | |
| | | ACAAACGACTGGTTCCGCGTGGTAGCTATTACGACTCCATTCCCACAAGCGAGA | |
| | | ACGACTCAGAAGTCAATCAAGAAGCTAAGCCAGAGGTCAAGCCAGAAGTCAAGC | |
| | | CTGAGACTCACATCAATTTAAAGGTGTCCGATGGATCTTCAGAGATCTTCTTCA | |
| | | AGATCAAAAAGACCACTCCTTTAAGAAGGCTGATGGAAGCGTTCGCTAAAAGAC | |
| | | AGGGTAAGGAAATGGACTCCTTAAGATTCTTGTACGACGGTATTAGAATTCAAG | |
| | | CTGATCAGGCCCCTGAAGATTTGGACATGGAGGATAACGATATTATTGAGGCTC | |
| | | ACCGCGAACAGATTGGAGGCTCCATTACAAGCCACCATCACCATCATCACGGTT | |
| | | AATACTTTCAGCCAAAAAACTTAAGACCGCCGGTCTTGTCCACTACCTTGCAGT | |
| | | AATGCGGTGGACAGGATCGGCGGTTTTCTTTTCTCTTCTCAACAAGTGAGACCA | |
| | | TGGgcggcgcgccatcgaatggcgcaaaacctttcgcggtatggcatgatagcg | |
| | | 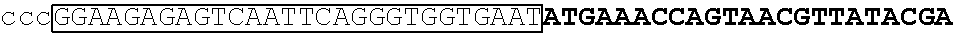 | |
| | | TGTCGCAGAGTATGCCGGTGTCTCTTATCAGACCGTTTCCCGCGTGGTGAACCA | |
| | | GGCCAGCCACGTTTCTGCGAAAACGCGGGAAAAAGTGGAAGCGGCGATGGCGGA | |
| | | GCTGAATTACATTCCCAACCGCGTGGCACAACAACTGGCGGGCAAACAGTCGTT | |
| | | GCTTATTGGCGTTGCCACCTCCAGTCTGGCCCTGCACGCGCCGTCGCAAATTGT | |
| | | CGCGGCGATTAAATCTCGCGCCGATCAACTGGGTGCCAGCGTGGTGGTGTCGAT | |
| | | GGTAGAACGAAGCGGCGTCGAAGCCTGTAAAGCGGCGGTGCACAATCTTCTCGC | |
| | | GCAACGCGTCAGTGGGCTGATCATTAACTATCCGCTGGATGACCAGGATGCCAT | |
| | | TGCTGTGGAAGCTGCCTGCACTAATGTTCCGGCGTTATTTCTTGATGTCTCTGA | |
| | | CCAGACACCCATCAACAGTATTATTTTCTCCCATGAGGACGGTACGCGACTGGG | |
| | | CGTGGAGCATCTGGTCGCATTGGGTCACCAGCAAATCGCGCTGTTAGCGGGCCC | |
| | | ATTAAGTTCTGTCTCGGCGCGTCTGCGTCTGGCTGGCTGGCATAAATATCTCAC | |
| | | TCGCAATCAAATTCAGCCGATAGCGGAACGGGAAGGCGACTGGAGTGCCATGTC | |
| | | CGGTTTTCAACAAACCATGCAAATGCTGAATGAGGGCATCGTTCCCACTGCGAT | |
| | | GCTGGTTGCCAACGATCAGATGGCGCTGGGCGCAATGCGCGCCATTACCGAGTC | |
| | | CGGGCTGCGCGTTGGTGCGGATATCTCGGTAGTGGGATACGACGATACCGAAGA | |
| | | TAGCTCATGTTATATCCCGCCGTTAACCACCATCAAACAGGATTTTCGCCTGCT | |
| | | GGGGCAAACCAGCGTGGACCGCTTGCTGCAACTCTCTCAGGGCCAGGCGGTGAA | |
| | | GGGCAATCAGCTGTTGCCAGTCTCACTGGTGAAAAGAAAAACCACCCTGGCGCC | |
| | | CAATACGCAAACCGCCTCTCCCCGCGCGTTGGCCGATTCATTAATGCAGCTGGC | |
| | | ACGACAGGTTTCCCGACTGGAAAGCGGGCAGTGATAATTGGTAACGAATCAGAC | |
| | | AATTGACGGCTCGAGGGAGTAGCATAGGGTTTGCAGAATCCCTGCTTCGTCCAT | |
| | | TTGACAGGCACATTATGCATCGATGATAAGCTGTCAAACATGAGCACGCTTACT | |
| | | AGTAGCGGCCGCTGCAGTCCGGCAAAAAAGGGCAAGGTGTCACCACCCTGCCCT | |
| | | TTTTCTTTAAAACCGAAAAGATTACTTCGCGTTATGCAGGCTTCCTCGCTCACT | |
| | | GACTCGCTGCGCTCGGTCGTTCGGCTGCGGCGAGCGGTATCAGCTCACTCAAAG | |
| | | GCGGTAATGACAGTAAGACGGGTAAGCCTGTTGATGATACCGCTGCCTTACTGG | |
| | | GTGCATTAGCCAGTCTGAATGACCTGTCACGGGATAATCCGAAGTGGTCAGACT | |
| | | GGAAAATCAGAGGGCAGGAACTGCTGAACAGCAAAAAGTCAGATAGCACCACAT | |
| | | AGCAGACCCGCCATAAAACGCCCTGAGAAGCCCGTGACGGGCTTTTCTTGTATT | |
| | | ATGGGTAGTTTCCTTGCATGAATCCATAAAAGGCGCCTGTAGTGCCATTTACCC | |
| | | CCATTCACTGCCAGAGCCGTGAGCGCAGCGAACTGAATGTCACGAAAAAGACAG | |
| | | CGACTCAGGTGCCTGATGGTCGGAGACAAAAGGAATATTCAGCGATTTGCCCGA | |
| | | GCTTGCGAGGGTGCTACTTAAGCCTTTAGGGTTTTAAGGTCTGTTTTGTAGAGG | |
| | | AGCAAACAGCGTTTGCGACATCCTTTTGTAATACTGCGGAACTGACTAAAGTAG | |
| | | TGAGTTATACACAGGGCTGGGATCTATTCTTTTTATCTTTTTTTATTCTTTCTT | |
| | | TATTCTATAAATTATAACCACTTGAATATAAACAAAAAAAACACACAAAGGTCT | |
| | | AGCGGAATTTACAGAGGGTCTAGCAGAATTTACAAGTTTTCCAGCAAAGGTCTA | |
| | | GCAGAATTTACAGATACCCACAACTCAAAGGAAAAGGACTAGTAATTATCATTG | |
| | | ACTAGCCCATCTCAATTGGTATAGTGATTAAAATCACCTAGACCAATTGAGATG | |
| | | TATGTCTGAATTAGTTGTTTTCAAAGCAAATGAACTAGCGATTAGTCGCTATGA | |
| | | CTTAACGGAGCATGAAACCAAGCTAATTTTATGCTGTGTGGCACTACTCAACCC | |
| | | CACGATTGAAAACCCTACAAGGAAAGAACGGACGGTATCGTTCACTTATAACCA | |
| | | ATACGCTCAGATGATGAACATCAGTAGGGAAAATGCTTATGGTGTATTAGCTAA | |
| | | AGCAACCAGAGAGCTGATGACGAGAACTGTGGAAATCAGGAATCCTTTGGTTAA | |
| | | AGGCTTTTGGATTTTCCAGTGGACAAACTATGCCAAGTTCTCAAGCGAAAAATT | |
| | | AGAATTAGTTTTTAGTGAAGAGATATTGCCTTATCTTTTCCAGTTAAAAAAATT | |
| | | CATAAAATATAATCTGGAACATGTTAAGTCTTTTGAAAACAAATACTCTATGAG | |
| | | GATTTATGAGTGGTTATTAAAAGAACTAACACAAAAGAAAACTCACAAGGCAAA | |
| | | TATAGAGATTAGCCTTGATGAATTTAAGTTCATGTTAATGCTTGAAAATAACTA | |
| | | CCATGAGTTTAAAAGGCTTAACCAATGGGTTTTGAAACCAATAAGTAAAGATTT | |
| | | AAACACTTACAGCAATATGAAATTGGTGGTTGATAAGCGAGGCCGCCCGACTGA | |
| | | TACGTTGATTTTCCAAGTTGAACTAGATAGACAAATGGATCTCGTAACCGAACT | |
| | | TGAGAACAACCAGATAAAAATGAATGGTGACAAAATACCAACAACCATTACATC | |
| | | AGATTCCTACCTACATAACGGACTAAGAAAAACACTACACGATGCTTTAACTGC | |
| | | AAAAATTCAGCTCACCAGTTTTGAGGCAAAATTTTTGAGTGACATGCAAAGTAA | |
| | | GTATGATCTCAATGGTTCGTTCTCATGGCTCACGCAAAAACAACGAACCACACT | |
| | | AGAGAACATACTGGCTAAATACGGAAGGATCTGAGGTTCTTATGGCTCTTGTAT | |
| | | CTATCAGTGAAGCATCAAGACTAACAAACAAAAGTAGAACAACTGTTCACCGTT | |
| | | ACATATCAAAGGGAAAACTGTCCATATGCACAGATGAAAACGGTGTAAAAAAGA | |
| | | TAGATACATCAGAGCTTTTACGAGTTTTTGGTGCATTCAAAGCTGTTCACCATG | |
| | | AACAGATCGACAATGTAACAGATGAACAGCATGTAACACCTAATAGAACAGGTG | |
| | | AAACCAGTAAAACAAAGCAACTAGAACATGAAATTGAACACCTGAGACAACTTG | |
| | | TTACAGCTCAACAGTCACACATAGACAGCCTGAAACAGGCGATGCTGCTTATCG | |
| | | AATCAAAGCTGCCGACAACACGGGAGCCAGTGACGCCTCCCGTGGGGAAAAAAT | |
| | | CATGGCAATTCTGGAAGAAATAGCGCTTTCAGCCGGCAAACCGGCTGAAGCCGG | |
| | | ATCTGCGATTCTGATAACAAACTAGCAACACCAGAACAGCCCGTTTGCGGGCAG | |
| | | CAAAACCCGTACCGATTATCAAAAAGGATCTTCACCTAGATCCTTTTAAATTAA | |
| | | AAATGAAGTTTTAAATCAATCTAAAGTATATATGAGTAAACTTGGTCTGACAGT | |
| | | TACCAATGCTTAATCAGTGAGGCACCTATCTCAGCGATCTGTCTATTTCGTTCA | |
| | | TCCATAGTTGCCTGACTCCCCGTCGTGTAGATAACTACGATACGGGAGGGCTTA | |
| | | CCATCTGGCCCCAGTGCTGCAATGATACCGCGAGAACCACGCTCACCGGCTCCA | |
| | | GATTTATCAGCAATAAACCAGCCAGCCGGAAGGGCCGAGCGCAGAAGTGGTCCT | |
| | | GCAACTTTATCCGCCTCCATCCAGTCTATTAATTGTTGCCGGGAAGCTAGAGTA | |
| | | AGTAGTTCGCCAGTTAATAGTTTGCGCAACGTTGTTGCCATTGCTACAGGCATC | |
| | | GTGGTGTCACGCTCGTCGTTTGGTATGGCTTCATTCAGCTCCGGTTCCCAACGA | |
| | | TCAAGGCGAGTTACATGATCCCCCATGTTGTGCAAAAAAGCGGTTAGCTCCTTC | |
| | | GGTCCTCCGATCGTTGTCAGAAGTAAGTTGGCCGCAGTGTTATCACTCATGGTT | |
| | | ATGGCAGCACTGCATAATTCTCTTACTGTCATGCCATCCGTAAGATGCTTTTCT | |
| | | GTGACTGGTGAGTACTCAACCAAGTCATTCTGAGAATAGTGTATGCGGCGACCG | |
| | | AGTTGCTCTTGCCCGGCGTCAATACGGGATAATACCGCGCCACATAGCAGAACT | |
| | | TTAAAAGTGCTCATCATTGGAAAACGTTCTTCGGGGCGAAAACTCTCAAGGATC | |
| | | TTACCGCTGTTGAGATCCAGTTCGATATAACCCACTCGTGCACCCAACTGATCT | |
| | | TCAGCATCTTTTACTTTCACCAGCGTTTCTGGGTGAGCAAAAACAGGAAGGCAA | |
| | | AATGCCGCAAAAAAGGGAATAAGGGCGACACGGAAATGTTGAATACTCATACTC | |
| | | TTCCTTTTTCAATATTATTGAAGCATTTATCAGGGTTATTGTCTCATGAGCGGA | |
| | | TACATATTTGAATGTATTTAGAAAAATAAACAAATAGGGGTTCCGCG | |
| pEG3212 | CapA | ATGGTGCGTTTCCTGGCTAAGCTGCTGCGTTCAACGATCCATGGCTCTAATGGC | 650 |
| | | GTGAGCCTCGACGCCGTCAGTTCCACGCATGGTACTCCGGGGTTTCAGACACCT | |
| | | GATGCACGTGTTATTTCACGCTTTGGCTTTAAT | |
| pEG3213 | LasA | ATGGACAAACGTGTGCGTTACGAAAAACCGAGCCTGGTGAAAGAGGGTACGTTT | 651 |
| | | CGCAAAACTACCGCTGGCCTGCGGCGTCTGTTCGCTGACCAGCTGGTTGGCCGC | |
| | | CGTAACATT | |
| pEG3214 | AlbsA | ATGGATTCACTGCTGTCAACAGAAACCGTCATTAGTGATGACGAACTGCTTCCG | 652 |
| | | ATTGAAGTTGGTGGTACCGCGGAATTGACAGAGGGGCAGGGCGGCGGTCAGTCC | |
| | | GAGGATAAACGTCGCGCTTATAACTGC | |
| pEG3215 | AtxAl | CCGATCATTAGCGAAACGGTCCAGCCTAAAACGGCTGGCCTGATTGTTCTGGGC | 653 |
| | | AAGGCAAGCGCGGAAACGCGCGGATTGAGCCAAGGCGTGGAACCGGACATTGGT | |
| | | CAGACGTACTTCGAAGAAAGCCGTATTAATCAGGAT | |
| pEG3553 | ClnlAl | ACTCCCATTCAATCCAAATTCTGCCTCCTGCGCGTGGGCAGTGCCAAACGGCTG | 654 |
| | | ACGCAGTCATTCGACGTGGGAACTATTAAGGAAGGTTTAGTCAGCCAGTATTAT | |
| | | TTTGCG | |
| pEG3554 | ClnlA2 | ACCCAGGTGAGCCCATCACCGCTGCGCCTGATTCGCGTCGGGAGAGCCTTGGAC | 655 |
| | | CTGACCCGCTCTATCGGGGATAGTGGGCTGCGTGAGTCCATGTCAAGCCAGACG | |
| | | TACTGGCCC | |
| pEG3555 | Cln2Al | AACACTTTAAAAACGCGTCTTATTCGCTTTGGGTCGGCTAAACGTCTGACGCGC | 656 |
| | | GCAGGTACGGGCGTGCTGTTACCTGAAACCAACCAGATTAAGCGCTACGATCCA | |
| | | GCA | |
| pEG3556 | Cln2A2 | ACCACACCCAAATTTCGACTGATTCGGTTAGGTTCAGCTAAGCGATTGACCCGG | 657 |
| | | TCGGGAATCGGGGATGTGTTTCCGGAGCCAAACATGGTTCGCCGCTGGGAT | |
| pEG3557 | Cln3Al | CAGCGTATAATAGATGAAACCACCGATGGTCTGATTGAACTGGGGGCGGCCAGC | 658 |
| | | GTACAGACACAGGGCGATGTTTTGTTTGCTCCGGAGCCTGGCGTGGGCCGACCT | |
| | | CCAATGGGCCTTTCCGAAGAT | |
| pEG3558 | Cln3A2 | GAACGCATTGAAGATCATATTGATGATGAACTGATTGACCTGGGAGCTGCTTCG | 659 |
| | | GTTGAAACCCAGGGAGATGTGCTGAATGCACCGGAGCCTGGTATCGGTCGTGAA | |
| | | CCGACAGGCTTGAGCCGCGAT | |
| pEG3559 | Cln3A3 | GAATTTGAAGGTATCCCATCACCGGATGCGCGTATTGATTTGGGTCTGGCGTCG | 660 |
| | | GAAGAAACCTGTGGTCAGATTTATGATCACCCGGAAGTAGGCATCGGTGCGTAC | |
| | | GGGTGCGAGGGCCTGCAGCGT | |
| pEG3560 | CsegAl | ACCAAGAAAAACGCAACACAGGCCCCACGTTTAGTACGTGTAGGCGATGCTCAT | 661 |
| | | CGTTTGACCCAAGGTGCTTTCGTTGGACAGCCGGAAGCCGTAAATCCACTTGGA | |
| | | CGTGAAATTCAAGGA | |
| pEG3561 | CsegA2 | ACCAAAACACACAGACTGATCAGATTGGGCGACGCGCAACGCTTGACCCAGGGC | 662 |
| | | ACATTGACTCCGGGCTTACCGGAGGACTTTCTGCCGGGCCATTACATGCCGGGG | |
| pEG3562 | CsegA3 | ACTTCACGTTTCCAACTCCTGCGCCTGGGAAAAGCCGATCGTTTGACGCGTGGC | 663 |
| | | GCGCTGGTCGGGCTCCTGATCGAAGATATTACTGTCGCTCGCTACGACCCTATG | |
| bEG_S7 | Lux Mod | pEG1128 below contains the full sequence of this | |
| | Backbone | backbone. | |
| pEG1128 | TruD | AACACCCCTTGTATTACTGTTTATGTAAGCAGACAGTTTTATTGTTCATGATGA | 664 |
| | | TATATTTTTATCTTGTGCAATGTACATCAGAGATTTTGAGACACAACCAATTAT | |
| | | TGAAGGCCTCCCTAACGGGGGGCCTTTTTTTGTTTCTGGTCTCCACGACGCTTA | |
| | | ACGATCGTTGGCTGacctgtaggatcgtacaggtttacgcaagaaaatggtttg | |
| | | ttacagtcgaataaaCAGCCCCATAGGGTGGTGTGTACCACCCCTGATGAGTCC | |
| | | 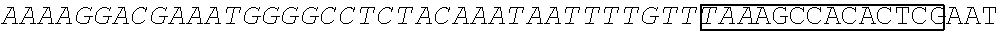 | |
| | | 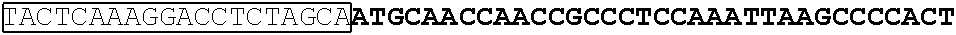 | |
| | | TCCACGTTGAGATAATTGAGCCGAAGCAAGTGTATCTCCTGGGCGAACAGGGCA | |
| | | ACCACGCTCTCACCGGGCAGCTCTACTGCCAAATTCTGCCTTTCTTAAACGGCG | |
| | | AATACACCCGAGAACAAATTGTGGAAAAGCTCGATGGGCAGGTCCCGGAGGAAT | |
| | | ATATCGACTTCGTACTCAGTCGTCTGGTGGAGAAGGGCTATCTAACTGAGGTGG | |
| | | CTCCAGAACTATCCCTGGAAGTGGCAGCATTTTGGAGCGAATTGGGAATTGCCC | |
| | | CTTCTGTAGTGGCAGAAGGGCTAAAGCAGCCAGTGACAGTGACAACGGCGGGCA | |
| | | AGGGCATTAGGGAAGGGATAGTGGCTAACCTGGCAGCAGCGCTGGAGGAAGCTG | |
| | | GCATTCAGGTGTCAGACCCAAGGGACCCAAAGGCCCCAAAGGCAGGGGATTCTA | |
| | | CTGCCCAGCTTCAGGTGGTGCTGACCGATGACTATTTACAGCCGGAACTTGCAG | |
| | | CGATCAACAAGGAAGCCTTAGAGCGCCAACAACCCTGGTTGCTGGTTAAGCCTG | |
| | | TGGGCAGTATCCTCTGGTTGGGACCGTTGTTCGTTCCTGGGGAAACCGGATGTT | |
| | | GGCACTGTCTTGCTCAACGATTGCAAGGCAACCGGGAAGTTGAAGCATCGGTAT | |
| | | TGCAACAAAAGCGAGCGCTGCAGGAGCGCAACGGTCAAAATAAAAATGGTGCAG | |
| | | TGAGTTGCTTGCCCACAGCACGGGCAACCCTACCTTCTACTCTACAAACAGGTT | |
| | | TACAGTGGGCTGCCACTGAGATTGCTAAGTGGATGGTCAAGCGGCACCTCAATG | |
| | | CCATAGCACCGGGAACGGCTCGTTTTCCCACTCTAGCTGGCAAGATATTTACAT | |
| | | TCAACCAGACGACTCTGGAGTTGAAAGCTCATCCTCTGAGCCGACGACCGCAAT | |
| | | GTCCCACCTGTGGCGATCGGGAAACTCTCCAACGGCGCGGGTTTGAACCACTGA | |
| | | AGCTAGAGTCGCGCCCCAAACACTTCACCTCCGATGGCGGTCATCGCGCCATGA | |
| | | CCCCAGAACAAACGGTGCAGAAGTACCAACACCTCATCGGGCCCATAACGGGGG | |
| | | TAGTGACGGAACTGGTGCGAATTTCTGACCCTGCCAATCCCTTGGTGCATACCT | |
| | | ACCGGGCTGGGCATAGCTTTGGCAGTGCTACGTCTCTGCGGGGGCTGCGCAATG | |
| | | TCCTACGCCACAAGAGTTCTGGTAAAGGCAAGACCGATAGCCAATCTCGGGCCA | |
| | | GCGGACTTTGCGAGGCGATCGAGCGCTATTCGGGCATTTTTCAGGGAGACGAAC | |
| | | CCCGCAAGCGGGCAACTTTGGCTGAGTTGGGAGATTTGGCGATTCATCCAGAAC | |
| | | AGTGTTTGCACTTTAGCGACAGGCAGTATGACAACCGGGAAAGCTCGAACGAGC | |
| | | GAGCAACAGTGACTCACGACTGGATTCCCCAACGGTTCGATGCAAGTAAGGCTC | |
| | | ACGACTGGACTCCCGTGTGGTCCCTAACGGAGCAAACCCATAAGTATCTGCCTA | |
| | | CAGCCCTGTGCTATTACCGATACCCCTTCCCCCCAGAACACCGTTTCTGCCGTA | |
| | | GTGACTCCAACGGAAACGCGGCGGGAAATACCCTGGAAGAGGCGATTTTGCAAG | |
| | | GATTTATGGAACTGGTGGAACGGGATAGCGTGTGCCTGTGGTGGTACAATCGCG | |
| | | TTAGCCGTCCGGCTGTGGATTTGAGTAGCTTTGACGAGCCTTATTTTTTGCAGT | |
| | | TGCAGCAGTTCTATCAAACTCAAAATCGCGATCTGTGGGTACTGGATTTAACAG | |
| | | CAGATTTGGGCATTCCGGCTTTTGTAGGGGTATCGAATCGGAAAGCCGGCAGCT | |
| | | CGGAAAGAATAATTCTCGGCTTTGGAGCGCACCTGGACCCGACAGTTGCCATCC | |
| | | TTCGCGCTCTTACGGAGGTCAACCAAATAGGCTTGGAATTGGATAAAGTTTCTG | |
| | | ATGAGAGCCTCAAGAACGATGCCACGGATTGGTTAGTGAATGCTACATTGGCAG | |
| | | CTAGTCCCTATCTCGTTGCCGATGCTAGCCAACCCCTCAAGACTGCGAAGGATT | |
| | | ATCCCCGGCGTTGGAGTGACGATATTTACACCGATGTGATGACTTGTGTAGAAA | |
| | | TAGCCAAGCAAGCAGGTCTAGAGACTTTGGTACTGGATCAGACCAGACCCGACA | |
| | | TAGGTTTAAATGTGGTTAAAGTCATTGTGCCAGGAATGCGTTTTTGGTCGCGAT | |
| | | TTGGCTCCGGTCGGCTCTATGACGTGCCAGTGAAGTTGGGATGGCGAGAGCAAC | |
| | | CACTTGCTGAGGCACAAATGAACCCTACACCGATGCCATTTTAATAAGATACGA | |
| | | ATTTATGTATAGACTCGGTACCAAAAAAAAAAAAAAAGACGCTGAAAAGCGTCT | |
| | | TTTTTTTTTTTGGTCCTACTATCCTTAAACGCATATCGTGGTACAGGAGACCGT | |
| | | CCAATGgcggcgcgccatcgaatggcgcaaaacctttcgcggtatggcatgata | |
| | | gcgcccggaagagagtcaattcagggtggtgaatATGAAAAACATAAATGCCGA | |
| | | CGACACATACAGAATAATTAATAAAATTAAAGCTTGTAGAAGCAATAATGATAT | |
| | | TAATCAATGCTTATCTGATATGACTAAAATGGTACATTGTGAATATTATTTACT | |
| | | CGCGATCATTTATCCTCATTCTATGGTTAAATCTGATATTTCAATCCTAGATAA | |
| | | TTACCCTAAAAAATGGAGGCAATATTATGATGACGCTAATTTAATAAAATATGA | |
| | | TCCTATAGTAGATTATTCTAACTCCAATCATTCACCAATTAATTGGAATATATT | |
| | | TGAAAACAATGCTGTAAATAAAAAATCTCCAAATGTAATTAAAGAAGCGAAAAC | |
| | | ATCAGGTCTTATCACTGGGTTTAGTTTCCCTATTCATACGGCTAACAATGGCTT | |
| | | CGGAATGCTTAGTTTTGCACATTCAGAAAAAGACAACTATATAGATAGTTTATT | |
| | | TTTACATGCGTGTATGAACATACCATTAATTGTTCCTTCTCTAGTTGATAATTA | |
| | | TCGAAAAATAAATATAGCAAATAATAAATCAAACAACGATTTAACCAAAAGAGA | |
| | | AAAAGAATGTTTAGCGTGGGCATGCGAAGGAAAAAGCTCTTGGGATATTTCAAA | |
| | | AATATTAGGTTGCAGTGAGCGTACTGTCACTTTCCATTTAACCAATGCGCAAAT | |
| | | GAAACTCAATACAACAAACCGCTGCCAAAGTATTTCTAAAGCAATTTTAACAGG | |
| | | AGCAATTGATTGCCCATACTTTAAAAATTGATAAGGATCCTAATTGGTAACGAA | |
| | | TCAGACAATTGACGGCTCGAGGGAGTAGCATAGGGTTTGCAGAATCCCTGCTTC | |
| | | GTCCATTTGACAGGCACATTATGCATCGATGATAAGCTGTCAAACATGAGCAGA | |
| | | TCCTCTACGCCGGACGCATCGTGGCCGGCATCACCGGCGCCACAGGTGCGGTTG | |
| | | CTGGCGCCTATATCGCCGACATCACCGATGGGGAAGATCGGGCTCGCCACTTCG | |
| | | GGCTCATGAGCAAATATTTTATCTGAGGTGCTTCCTCGCTCACTGACTCGCTGC | |
| | | ACGAGGCAGACCTCAGCGCTAGCGGAGTGTATACTGGCTTACTATGTTGGCACT | |
| | | GATGAGGGTGTCAGTGAAGTGCTTCATGTGGCAGGAGAAAAAAGGCTGCACCGG | |
| | | TGCGTCAGCAGAATATGTGATACAGGATATATTCCGCTTCCTCGCTCACTGACT | |
| | | CGCTACGCTCGGTCGTTCGACTGCGGCGAGCGGAAATGGCTTACGAACGGGGCG | |
| | | GAGATTTCCTGGAAGATGCCAGGAAGATACTTAACAGGGAAGTGAGAGGGCCGC | |
| | | GGCAAAGCCGTTTTTCCATAGGCTCCGCCCCCCTGACAAGCATCACGAAATCTG | |
| | | ACGCTCAAATCAGTGGTGGCGAAACCCGACAGGACTATAAAGATACCAGGCGTT | |
| | | TCCCCTGGCGGCTCCCTCGTGCGCTCTCCTGTTCCTGCCTTTCGGTTTACCGGT | |
| | | GTCATTCCGCTGTTATGGCCGCGTTTGTCTCATTCCACGCCTGACACTCAGTTC | |
| | | CGGGTAGGCAGTTCGCTCCAAGCTGGACTGTATGCACGAACCCCCCGTTCAGTC | |
| | | CGACCGCTGCGCCTTATCCGGTAACTATCGTCTTGAGTCCAACCCGGAAAGACA | |
| | | TGCAAAAGCACCACTGGCAGCAGCCACTGGTAATTGATTTAGAGGAGTTAGTCT | |
| | | TGAAGTCATGCGCCGGTTAAGGCTAAACTGAAAGGACAAGTTTTGGTGACTGCG | |
| | | CTCCTCCAAGCCAGTTACCTCGGTTCAAAGAGTTGGTAGCTCAGAGAACCTTCG | |
| | | AAAAACCGCCCTGCAAGGCGGTTTTTTCGTTTTCAGAGCAAGAGATTACGCGCA | |
| | | GACCAAAACGATCTCAAGAAGATCATCTTATTAAGGGGTCTGACGCTCAGTGGA | |
| | | ACGAAAAATCAATCTAAAGTATATATGAGTAAACTTGGTCTGACAGTTACCTTA | |
| | | GAAAAACTCATCGAGCATCAAATGAAACTGCAATTTATTCATATCAGGATTATC | |
| | | AATACCATATTTTTGAAAAAGCCGTTTCTGTAATGAAGGAGAAAACTCACCGAG | |
| | | GCAGTTCCATAGGATGGCAAGATCCTGGTATCGGTCTGCGATTCCGACTCGTCC | |
| | | AACATCAATACAACCTATTAATTTCCCCTCGTCAAAAATAAGGTTATCAAGTGA | |
| | | GAAATCACCATGAGTGACGACTGAATCCGGTGAGAATGGCAAAAGCTTATGCAT | |
| | | TTCTTTCCAGACTTGTTCAACAGGCCAGCCATTACGCTCGTCATCAAAATCACT | |
| | | CGCATCAACCAAACCGTTATTCATTCGTGATTGCGCCTGAGCGAGACGAAATAC | |
| | | GCGATCGCTGTTAAAAGGACAATTACAAACAGGAATCGAATGCAACCGGCGCAG | |
| | | GAACACTGCCAGCGCATCAACAATATTTTCACCTGAATCAGGATATTCTTCTAA | |
| | | TACCTGGAATGCTGTTTTCCCGGGGATCGCAGTGGTGAGTAACCATGCATCATC | |
| | | AGGAGTACGGATAAAATGCTTGATGGTCGGAAGAGGCATAAATTCCGTCAGCCA | |
| | | GTTTAGTCTGACCATCTCATCTGTAACATCATTGGCAACGCTACCTTTGCCATG | |
| | | TTTCAGAAACAACTCTGGCGCATCGGGCTTCCCATACAATCGATAGATTGTCGC | |
| | | ACCTGATTGCCCGACATTATCGCGAGCCCATTTATACCCATATAAATCAGCATC | |
| | | CATGTTGGAATTTAATCGCGGCCTCGAGCAAGACGTTTCCCGTTGAATATGGCT | |
| | | CAT | |
| bEG_S8 | Cym Mod | AACACCCCTTGTATTACTGTTTATGTAAGCAGACAGTTTTATTGTTCATGATGA | 665 |
| | Backbone | TATATTTTTATCTTGTGCAATGTACATCAGAGATTTTGAGACACAACCAATTAT | |
| | without | TGAAGGCCTCCCTAACGGGGGGCCTTTTTTTGTTTCTGGTCTCCCAAGCGCTTA | |
| | SapI sites | ACGATCGTTGGCTGaacaaacagacaatctggtctgtttgtattatggaaaatt | |
| | around | tttctgtataatagattcaacaaacagacaatctggtctgtttgtattatCAGC | |
| | CDS | GGTCAACGCATGTGCTTTGCGTTCTGATGAGACAGTGATGTCGAAACCGCCTCT | |
| | | 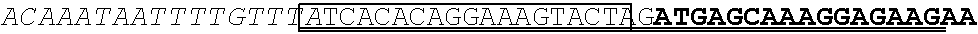 | |
| | | CTTTTCACTGGAGTTGTCCCAATTCTTGTTGAATTAGATGGTGATGTTAATGGG | |
| | | CACAAATTTTCTGTCCGTGGAGAGGGTGAAGGTGATGCTACAAACGGAAAACTC | |
| | | ACCCTTAAATTTATTTGCACTACTGGAAAACTACCTGTTCCGTGGCCAACACTT | |
| | | GTCACTACTCTGACCTATGGTGTTCAATGCTTTTCCCGTTATCCGGATCACATG | |
| | | AAACGGCATGACTTTTTCAAGAGTGCCATGCCCGAAGGTTATGTACAGGAACGC | |
| | | ACTATATCTTTCAAAGATGACGGGACCTACAAGACGCGTGCTGAAGTCAAGTTT | |
| | | GAAGGTGATACCCTTGTTAATCGTATCGAGTTAAAGGGTATTGATTTTAAAGAA | |
| | | GATGGAAACATTCTTGGACACAAACTCGAGTACAACTTTAACTCACACAATGTA | |
| | | TACATCACGGCAGACAAACAAAAGAATGGAATCAAAGCTAACTTCAAAATTCGC | |
| | | CACAACGTTGAAGATGGTTCCGTTCAACTAGCAGACCATTATCAACAAAATACT | |
| | | CCAATTGGCGATGGCCCTGTCCTTTTACCAGACAACCATTACCTGTCGACACAA | |
| | | TCTGTCCTTTCGAAAGATCCCAACGAAAAGCGTGACCACATGGTCCTTCTTGAG | |
| | | TTTGTAACTGCTGCTGGGATTACACATGGCATGGATGAGCTCTACAAATAAGGA | |
| | | TGAGCTCTACAAATAAGCAGAGGTGGTTGTGTTGCGAAAAAAAAAAAAAAACAC | |
| | | CCTAACGGGTGTTTTTTTTTTTTTGGTGTCCCCACGTGTGGCGCTGGAGACCGT | |
| | | CCAATGgcggcgcgccatcgaatggtgcaaaacctttcgcggtatggcatgata | |
| | | 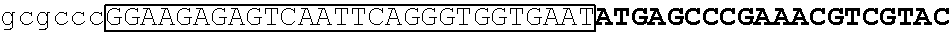 | |
| | | CCAGGCAGAACGTGCAATGGAAACCCAGGGTAAACTGATTGCAGCAGCACTGGG | |
| | | TGTTCTGCGTGAAAAAGGTTATGCAGGTTTTCGTATTGCAGATGTTCCGGGTGC | |
| | | AGCCGGTGTTAGCCGTGGTGCACAGAGCCATCATTTTCCGACCAAACTGGAACT | |
| | | GCTGCTGGCAACCTTTGAATGGCTGTATGAGCAGATTACCGAACGTAGCCGTGC | |
| | | ACGTCTGGCAAAACTGAAACCGGAAGATGATGTTATTCAGCAGATGCTGGATGA | |
| | | TGCAGCAGATTTTTTTCTGGATGATGATTTTAGCATCGGCCTGGATCTGATTGT | |
| | | TGCAGCAGATCGTGATCCGGCACTGCGTGAAGGTATTCTGCGTACCGTTGAACG | |
| | | TAATCGTTTTGTTGTTGAAGATATGTGGCTGGGTGTGCTGGTGAGCCGTGGTCT | |
| | | GAGCCGTGATGATGCCGAAGATATTCTGTGGCTGATTTTTAACAGCGTTCGTGG | |
| | | TCTGACAGTTCGTAGCCTGTGGCAGAAAGATAAAGAACGTTTTGAACGTGTGCG | |
| | | TAATAGCACCCTGGAAATTGCACGTGAACGTTATGCAAAATTCAAACGTTGATA | |
| | | AGGATCCTAATTGGTAACGAATCAGACAATTGACGGCTCGAGGGAGTAGCATAG | |
| | | GGTTTGCAGAATCCCTGCTTCGTCCATTTGACAGGCACATTATGCATCGATGAT | |
| | | AAGCTGTCAAACATGAGCAGATCCTCTACGCCGGACGCATCGTGGCCGGCATCA | |
| | | CCGGCGCCACAGGTGCGGTTGCTGGCGCCTATATCGCCGACATCACCGATGGGG | |
| | | AAGATCGGGCTCGCCACTTCGGGCTCATGAGCAAATATTTTATCTGAGGTGCTT | |
| | | CCTCGCTCACTGACTCGCTGCACGAGGCAGACCTCAGCGCTAGCGGAGTGTATA | |
| | | CTGGCTTACTATGTTGGCACTGATGAGGGTGTCAGTGAAGTGCTTCATGTGGCA | |
| | | GGAGAAAAAAGGCTGCACCGGTGCGTCAGCAGAATATGTGATACAGGATATATT | |
| | | CCGCTTCCTCGCTCACTGACTCGCTACGCTCGGTCGTTCGACTGCGGCGAGCGG | |
| | | AAATGGCTTACGAACGGGGCGGAGATTTCCTGGAAGATGCCAGGAAGATACTTA | |
| | | ACAGGGAAGTGAGAGGGCCGCGGCAAAGCCGTTTTTCCATAGGCTCCGCCCCCC | |
| | | TGACAAGCATCACGAAATCTGACGCTCAAATCAGTGGTGGCGAAACCCGACAGG | |
| | | ACTATAAAGATACCAGGCGTTTCCCCTGGCGGCTCCCTCGTGCGCTCTCCTGTT | |
| | | CCTGCCTTTCGGTTTACCGGTGTCATTCCGCTGTTATGGCCGCGTTTGTCTCAT | |
| | | TCCACGCCTGACACTCAGTTCCGGGTAGGCAGTTCGCTCCAAGCTGGACTGTAT | |
| | | GCACGAACCCCCCGTTCAGTCCGACCGCTGCGCCTTATCCGGTAACTATCGTCT | |
| | | TGAGTCCAACCCGGAAAGACATGCAAAAGCACCACTGGCAGCAGCCACTGGTAA | |
| | | TTGATTTAGAGGAGTTAGTCTTGAAGTCATGCGCCGGTTAAGGCTAAACTGAAA | |
| | | GGACAAGTTTTGGTGACTGCGCTCCTCCAAGCCAGTTACCTCGGTTCAAAGAGT | |
| | | TGGTAGCTCAGAGAACCTTCGAAAAACCGCCCTGCAAGGCGGTTTTTTCGTTTT | |
| | | CAGAGCAAGAGATTACGCGCAGACCAAAACGATCTCAAGAAGATCATCTTATTA | |
| | | AGGGGTCTGACGCTCAGTGGAACGAAAAATCAATCTAAAGTATATATGAGTAAA | |
| | | CTTGGTCTGACAGTTACCTTAGAAAAACTCATCGAGCATCAAATGAAACTGCAA | |
| | | TTTATTCATATCAGGATTATCAATACCATATTTTTGAAAAAGCCGTTTCTGTAA | |
| | | TGAAGGAGAAAACTCACCGAGGCAGTTCCATAGGATGGCAAGATCCTGGTATCG | |
| | | GTCTGCGATTCCGACTCGTCCAACATCAATACAACCTATTAATTTCCCCTCGTC | |
| | | AAAAATAAGGTTATCAAGTGAGAAATCACCATGAGTGACGACTGAATCCGGTGA | |
| | | GAATGGCAAAAGCTTATGCATTTCTTTCCAGACTTGTTCAACAGGCCAGCCATT | |
| | | ACGCTCGTCATCAAAATCACTCGCATCAACCAAACCGTTATTCATTCGTGATTG | |
| | | CGCCTGAGCGAGACGAAATACGCGATCGCTGTTAAAAGGACAATTACAAACAGG | |
| | | AATCGAATGCAACCGGCGCAGGAACACTGCCAGCGCATCAACAATATTTTCACC | |
| | | TGAATCAGGATATTCTTCTAATACCTGGAATGCTGTTTTCCCGGGGATCGCAGT | |
| | | GGTGAGTAACCATGCATCATCAGGAGTACGGATAAAATGCTTGATGGTCGGAAG | |
| | | AGGCATAAATTCCGTCAGCCAGTTTAGTCTGACCATCTCATCTGTAACATCATT | |
| | | GGCAACGCTACCTTTGCCATGTTTCAGAAACAACTCTGGCGCATCGGGCTTCCC | |
| | | ATACAATCGATAGATTGTCGCACCTGATTGCCCGACATTATCGCGAGCCCATTT | |
| | | ATACCCATATAAATCAGCATCCATGTTGGAATTTAATCGCGGCCTCGAGCAAGA | |
| | | CGTTTCCCGTTGAATATGGCTCAT | |
| pEG7172 | HalM1 | 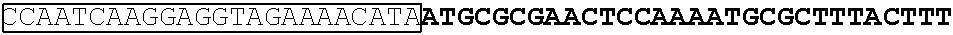 | 666 |
| | | AGCGAAGTGGTTTTTGGACCGAATCTTGAGAAGATTGTAGGAGAAAAGCGCCTC | |
| | | AATTTTTGGCTCAAACTTATAGGTGAGGACCCGGAAAACCTGAAGGAGTTTCTC | |
| | | TCGAGAAAGGGCAATTCTTTCGAAGAACAAACCTTACCGGAAAAGGAAGCTATC | |
| | | GTTCCGAACCGCTTAGGTGAAGAGGCGCTGGAAAAAGTCCGCGAAGAACTTGAG | |
| | | TTCCTCAATACTTACAGCACTAAACATGTGCGTCGCGTTAAAGAGTTGGGAGTG | |
| | | CAGATCCCTTTCGAAGGGATTCTGCTGCCATTCATTAGCATGTATATCGAAAAA | |
| | | TTTCAGCAGCAGCAACTTCGCAAAAAGATAGGGCCGATTCACGAAGAGATCTGG | |
| | | ACGCAGATTGTTCAAGATATCACCTCCAAATTAAATGCGATTCTGCACCGTACC | |
| | | CTGATCCTGGAACTGAATGTAGCTCGTGTTACCTCCCAACTTAAAGGTGATACT | |
| | | CCGGAAGAAAGATTCGCCTACTACTCGAAAACCTATTTAGGCAAACGTGAAGTA | |
| | | ACTCACCGTCTGTATAGCGAATATCCGGTGGTTCTGCGGTTGCTGTTCACCACC | |
| | | ATTTCACACCACATTTCGTTCATTACGGAAATCCTTGAACGCGTTGCAAATGAC | |
| | | CGTGAAGCCATTGAAACCGAATTTTCACCGTGTTCCCCGATTGGTACCCTCGCC | |
| | | TCTCTCCACTTAAACTCGGGAGATGCTCACCATAAACAGCGTACTGTGACGATT | |
| | | TTGGAATTCTCCTCCTCGCTGAAACTTGTCTACAAACCTCGCTCCCTCAAAGTT | |
| | | GATGGGGTGTTCAACGGTTTACTCGCTTTCCTGAACGATAGAACGGGGGAAGTC | |
| | | ATTAAGGACCAGTATTGCCCTAAGGTGTTACAGCGCGATGGCTACGGCTATGTG | |
| | | GAATTTGTCACTCACCAGTCTTGTCAATCCCTTGAGGAAGTGTCAGACTTCTAC | |
| | | GAGAGACTCGGCTCTCTGATGAGTCTGTCCTACGTACTGAATAGTTCTGACTTT | |
| | | CATTTCGAGAACATTATAGCTCATGGTCCCTATCCTGTCCTGATCGATCTTGAA | |
| | | ACCATCATTCATAATACAGCGGATAGCAGCGAGGAAACGTCTACCGCTATGGAT | |
| | | CGCGCGTTCCGTATGTTGAACGATTCGGTGCTGTCCACTGGTATGCTTCCCTCC | |
| | | TCTATTTATTATCGCGATCAGCCGAATATGAAGGGTCTGAACGTCGGAGGTGTG | |
| | | AGCAAATCAGAAGGTCAGAAAACACCGTTCAAAGTTAATCAAATCGCCAATCGC | |
| | | AACACCGATGAGATGCGTATCGAAAAAGATCACGTTACCCTGAGCAGCCAGAAA | |
| | | AATCTGCCCATTTTTCAGTCTGCCGCAATGGAGAGCGTACATTTCTTAGATCAG | |
| | | ATCCAGAAAGGCTTTACCTCCATGTATCAGTGGATCGAGAAGAACAAACAAGAA | |
| | | TTTAAAGAACAGGTGCGTAAGTTTGAAGGTGTGCCGGTTCGTGCTGTTCTTCGG | |
| | | AGCACGACTCGCTATACCGAACTGCTGAAATCTTCCTACCACCCTGACCTGCTC | |
| | | CGCAGCGCGTTGGACCGTGAAGTACTGCTGAACCGTTTGACTGTTGACTCGGTA | |
| | | ATGACCCCGTATCTCAAAGAGATTATTCCACTCGAGGTGGAAGATCTGCTGAAC | |
| | | GGTGACGTGCCATACTTCTACACCCTGCCGGAAGAACGCGCCCTGTATCAGGAA | |
| | | GCGTCTGCGATCAATAGTACGTTCTTTACCACTTCGATTTTCCATAAGATTGAC | |
| | | CAGAAAATCGATAAGCTGGGTATCGAGGACCATACCCAGCAAATGAAGATCTTA | |
| | | CACATGAGTATGCTTGCCTCTAACGCTAACCATTACGCCGATGTTGCCGACTTG | |
| | | GATATTCAGAAAGGACACACCATTAAAAACGAACAGTACGTTGAGATGGCCAAA | |
| | | GACATCGGTGATTACCTGATGGAGTTATCGGTCGAGGGTGAAAATCAAGGGGAA | |
| | | CCAGATCTGTGTTGGATTTCGACCGTCCTGGAAGGGAGCTCTGAAATCATTTGG | |
| | | GACATCAGCCCAGTGGGCGAAGATTTATACAACGGCAGCGCTGGCGTCGCTCTC | |
| | | TTTTATGCGTACCTGTTCAAAATTACAGGTGAAAAGCGTTACCAAGAGATCGCA | |
| | | TACAAAGCCCTGGTTCCGGTTCGCCGCAGTGTGGCCCAATTCCAGCACCATCCG | |
| | | AATTGGAGCATTGGTGCGTTTAACGGAGCGTCAGGCTATCTGTACGCGATGGGT | |
| | | ACGATAGCGGCCCTGTTTAATGATGAACGTTTGAAGCATGAAGTAACCCGCAGC | |
| | | ATTCCGCACATTGAACCGATGATCCACGAGGATAAGATCTATGATTTCATTGGC | |
| | | GGTTCCGCAGGGGCGCTGAAGGTGTTCCTGAGCCTGTCGGGGCTGTTTGACGAG | |
| | | CCGAAGTTTTTGGAACTTGCCATTGCATGCAGCGAACATCTGATGAAAAACGCC | |
| | | ATTAAAACGGATCAAGGTATCGGCTGGAAACCACCGTGGGAGGTCACCCCACTG | |
| | | ACCGGTTTCAGCCATGGGGTTAGCGGCGTCATGGCATCCTTCATCGAACTGTAC | |
| | | CAGCAAACCGGTGATGAGCGCTTGCTCAGTTACATTGATCAGAGTTTAGCCTAT | |
| | | GAACGTTCCTTCTTCAGCGAACAAGAGGAGAACTGGCTGACTCCGAACAAAGAA | |
| | | ACACCCGTGGTAGCTTGGTGCCACGGCGCGCCGGGAATTTTGGTATCACGACTG | |
| | | CTTCTGAAGAAATGCGGCTATTTGGATGAAAAAGTCGAAAAAGAAATTGAGGTG | |
| | | GCATTATCCACAACTATCCGTAAAGGCCTTGGTAACAATCGCAGTCTTTGCCAT | |
| | | GGTGATTTCGGCCAGCTGGAAATTCTTCGCTTTGCGGCGGAAGTGTTAGGCGAT | |
| | | AGCTATCTCCAGGAAGTTGTCAACAATCTGTCCGGCGAGTTGTATAATCTTTTC | |
| | | AAAACGGAGGGATATCAGAGCGGAACCAGCCGCGGTACTGAATCCGTGGGCCTG | |
| | | ATGGTAGGTCTGTCCGGGTTTGGGTATGGTTTACTTTCAGCGGCATATCCATCT | |
| | | GCTGTCCCCTCAATCTTAACATTGGATGGTGAGATCCAGAAGTACCGGGAGCCT | |
| | | CATGAAGCCTGA | |
| pEG7173 | HalM2 | 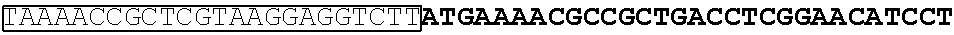 | 667 |
| | | TCAGTGCCGACGACGCTGCCGCATACTAACGACACCGATTGGCTCGAGCAATTA | |
| | | CATGACATTTTGTCCATTCCTGTTACGGAAGAAATCCAGAAATATTTCCACGCC | |
| | | GAAAATGATCTGTTCTCGTTTTTCTATACACCGTTCCTGCAGTTTACGTACCAG | |
| | | AGCATGTCGGACTACTTTATGACCTTCAAGACCGATATGGCCCTGATCGAAAGA | |
| | | CAGAGCCTCCTGCAAAGCACGCTGACCGCGGTACATCACCGACTCTTCCACTTA | |
| | | ACGCATCGCACCCTTATTAGTGAAATGCATATTGATAAACTTACCGTTGGCCTG | |
| | | AATGGCTCTACGCCGCACGAGCGCTACATGGATTTCAACCACAAATTCAACAAA | |
| | | ACCTCGAAGTCGAAGAACCTGTTTAACATCTACCCAATTTTGGGAAAATTGGTC | |
| | | GTTAACGAAACTCTGCGCACTATTAACTTCGTCAAGAAAATCATTCAGCACTAC | |
| | | ATGAAGGACTACCTGCTCCTGTCGGACTTCTTCAAAGAGAAGGACTTGCGTCTT | |
| | | ACCAACCTGCAATTAGGCGTGGGGGATACACACGTTAATGGGCAATGCGTCACC | |
| | | ATTCTGACGTTTGCATCAGGCCAAAAAGTGGTATACAAACCTAGATCATTGTCG | |
| | | ATAGATAAACAGTTCGGAGAATTCATCGAGTGGGTAAACTCGAAAGGTTTTCAG | |
| | | CCTTCCTTGCGTATCCCTATTGCGATTGATCGTCAAACCTATGGTTGGTATGAA | |
| | | TTCATCCCTCATCAAGAGGCCACCAGCGAAGATGAAATAGAACGCTACTATTCT | |
| | | CGCATCGGTGGTTATCTGGCGATCGCCTACTTGTTCGGGGCAACCGACCTGCAC | |
| | | CTGGATAACCTGATCGCCTGCGGCGAACATCCGATGCTTATTGATTTGGAAACA | |
| | | CTCTTTACCAACGATCTCGACTGCTATGACAGTGCGTTTCCGTTCCCGGCGCTG | |
| | | GCCCGCGAATTAACCCAATCCGTTTTTGGCACCCTTATGCTTCCCATCACCATC | |
| | | GCGTCGGGGAAACTGCTGGATATAGACCTGTCAGCAGTAGGAGGCGGTAAAGGT | |
| | | GTGCAGTCCGAAAAGATCAAAACCTGGGTCATCGTGAATCAGAAAACTGATGAG | |
| | | ATGAAGCTGGTCGAGCAGCCGTATGTTACCGAGAGTTCCCAGAATAAACCAACA | |
| | | GTTAATGGGAAAGAGGCGAACATTGGCAATTATATTCCTCATGTCACAGATGGC | |
| | | TTTCGTAAAATGTACCGCCTGTTTCTGAATGAAATTGATGAGTTAATGGATCAT | |
| | | AACGGGCCAATCTTTGCGTTTGAGAGTTGTCAGATTCGTCATGTTTTTCGAGCT | |
| | | ACCCACGTGTATGCGAAATTTTTGGAGGCAAGTACCCACCCAGATTACTTGCAA | |
| | | GAACCTACCAGACGTAATAAACTGTTCGAGTCCTTTTGGAACATCACGTCGCTG | |
| | | ATGGCACCGTTCAAGAAAATTGTACCGCACGAAATCGCGGAGTTGGAGAACCAT | |
| | | GATATTCCGTACTTCGTCCTGACTTGTGGCGGCACCATTGTTAAAGATGGATAC | |
| | | GGCCGGGATATCGCAGACCTGTTTCAAAGTAGCTGCATCGAACGTGTAACTCAT | |
| | | CGTCTGCAGCAGCTGGGAAGCGAGGATGAGGCGCGTCAAATTCGCTACATTAAA | |
| | | AGCAGCCTGGCGACGTTGACCAACGGTGATTGGACCCCATCCCATGAGAAAACC | |
| | | CCGATGTCTCCGGCCTCGGCCGACCGTGAAGATGGTTACTTCCTGCGCGAGGCT | |
| | | CAGGCCATCGGCGACGACATTTTGGCGCAGCTGATTTGGGAGGATGACCGTCAC | |
| | | GCCGCTTACCTTATTGGCGTAAGCGTGGGCATGAACGAAGCCGTCACTGTGTCA | |
| | | CCCCTGACGCCTGGCATCTACGACGGCACACTTGGCATAGTGCTGTTCTTCGAT | |
| | | CAGCTGGCCCAGCAGACCGGCGAAACCCATTATCGCCACGCCGCCGACGCTTTA | |
| | | CTGGAAGGAATGTTCAAACAGCTGAAACCTGAACTGATGCCGTCTAGCGCTTAC | |
| | | TTCGGACTGGGTAGCCTGTTCTATGGCCTGATGGTGTTGGGCCTCCAGCGTTCC | |
| | | GACTCGCATATCATTCAGAAAGCGTATGAGTATCTGAAACATTTGGAAGAGTGT | |
| | | GTGCAGCATGAGGAAACGCCAGATTTTGTCTCGGGTTTGTCTGGTGTACTGTAT | |
| | | ATGCTCACGAAAATTTATCAGCTCACGAATGAACCGAGAGTTTTCGAAGTGGCC | |
| | | AAAACCACAGCTTCGCGTCTGTCTGTGCTGCTTGACAGCAAGCAGCCCGACACT | |
| | | GTGCTCACCGGGTTATCCCATGGCGCCGCAGGATTCGCCCTTGCATTACTGACC | |
| | | TACGGAACCGCTGCAAATGATGAACAGTTGCTGAAACAGGGCCACTCCTATCTG | |
| | | GTGTACGAACGTAATCGGTTTAACAAACAGGAAAACAACTGGGTTGATTTACGT | |
| | | AAAGGCAACGCGTATCAAACATTTTGGTGCCATGGCGCCCCGGGTATTGGCATC | |
| | | TCACGCCTCCTGTTAGCGCAATTTTACGATGACGAACTGCTGCATGAAGAGTTA | |
| | | AACGCAGCACTGAACAAGACTATTTCGGACGGCTTCGGCCACAATCACTCACTG | |
| | | TGTCATGGCGATTTCGGCAACCTCGATCTGTTATTGCTTTATGCCCAATATACG | |
| | | AATAACCCAGAACCAAAGGAACTCGCTCGCAAACTGGCCATAAGCAGTATCGAT | |
| | | CAAGCGCACACGTATGGCTGGAAACTCGGGCTCAATCATAGCGATCAACTGCAG | |
| | | GGTATGATGTTAGGGGTGACTGGTATCGGCTATCAGCTCCTTCGTCATATAAAT | |
| | | CCGACAGTCCCCAGCATTTTGGCACTGGAACTGCCCAGCTCCACGTTAACTGAA | |
| | | AAAGAGCTGAGAATCCATGATCGTTGATAA | |
| bEG_S9 | Cym Mod | AACACCCCTTGTATTACTGTTTATGTAAGCAGACAGTTTTATTGTTCATGATGA | 668 |
| | Backbone | TATATTTTTATCTTGTGCAATGTACATCAGAGATTTTGAGACACAACCAATTAT | |
| | | TGAAGGCCTCCCTAACGGGGGGCCTTTTTTTGTTTCTGGTCTCCCAAGCGCTTA | |
| | | ACGATCGTTGGCTGaacaaacagacaatctggtctgtttgtattatggaaaatt | |
| | | tttctgtataatagattcaacaaacagacaatctggtctgtttgtattatCAGC | |
| | | GGTCAACGCATGTGCTTTGCGTTCTGATGAGACAGTGATGTCGAAACCGCCTCT | |
| | | 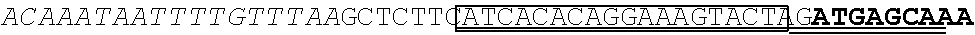 | |
| | | GGAGAAGAACTTTTCACTGGAGTTGTCCCAATTCTTGTTGAATTAGATGGTGAT | |
| | | GTTAATGGGCACAAATTTTCTGTCCGTGGAGAGGGTGAAGGTGATGCTACAAAC | |
| | | GGAAAACTCACCCTTAAATTTATTTGCACTACTGGAAAACTACCTGTTCCGTGG | |
| | | CCAACACTTGTCACTACTCTGACCTATGGTGTTCAATGCTTTTCCCGTTATCCG | |
| | | GATCACATGAAACGGCATGACTTTTTCAAGAGTGCCATGCCCGAAGGTTATGTA | |
| | | CAGGAACGCACTATATCTTTCAAAGATGACGGGACCTACAAGACGCGTGCTGAA | |
| | | GTCAAGTTTGAAGGTGATACCCTTGTTAATCGTATCGAGTTAAAGGGTATTGAT | |
| | | TTTAAAGAAGATGGAAACATTCTTGGACACAAACTCGAGTACAACTTTAACTCA | |
| | | CACAATGTATACATCACGGCAGACAAACAAAAGAATGGAATCAAAGCTAACTTC | |
| | | AAAATTCGCCACAACGTTGAAGATGGTTCCGTTCAACTAGCAGACCATTATCAA | |
| | | CAAAATACTCCAATTGGCGATGGCCCTGTCCTTTTACCAGACAACCATTACCTG | |
| | | TCGACACAATCTGTCCTTTCGAAAGATCCCAACGAAAAGCGTGACCACATGGTC | |
| | | CTTCTTGAGTTTGTAACTGCTGCTGGGATTACACATGGCATGGATGAGCTCTAC | |
| | | AAATAATGAAGAGCGCAGAGGTGGTTGTGTTGCGAAAAAAAAAAAAAACACCCT | |
| | | AACGGGTGTTTTTTTTTTTTTGGTGTCCCCACGTGTGGCGCTGGAGACCGTCCA | |
| | | ATGgcggcgcgccatcgaatggtgcaaaacctttcgcggtatggcatgatagcg | |
| | | 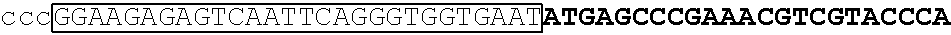 | |
| | | GGCAGAACGTGCAATGGAAACCCAGGGTAAACTGATTGCAGCAGCACTGGGTGT | |
| | | TCTGCGTGAAAAAGGTTATGCAGGTTTTCGTATTGCAGATGTTCCGGGTGCAGC | |
| | | CGGTGTTAGCCGTGGTGCACAGAGCCATCATTTTCCGACCAAACTGGAACTGCT | |
| | | GCTGGCAACCTTTGAATGGCTGTATGAGCAGATTACCGAACGTAGCCGTGCACG | |
| | | TCTGGCAAAACTGAAACCGGAAGATGATGTTATTCAGCAGATGCTGGATGATGC | |
| | | AGCAGATTTTTTTCTGGATGATGATTTTAGCATCGGCCTGGATCTGATTGTTGC | |
| | | AGCAGATCGTGATCCGGCACTGCGTGAAGGTATTCTGCGTACCGTTGAACGTAA | |
| | | TCGTTTTGTTGTTGAAGATATGTGGCTGGGTGTGCTGGTGAGCCGTGGTCTGAG | |
| | | CCGTGATGATGCCGAAGATATTCTGTGGCTGATTTTTAACAGCGTTCGTGGTCT | |
| | | GACAGTTCGTAGCCTGTGGCAGAAAGATAAAGAACGTTTTGAACGTGTGCGTAA | |
| | | TAGCACCCTGGAAATTGCACGTGAACGTTATGCAAAATTCAAACGTTGATAAGG | |
| | | ATCCTAATTGGTAACGAATCAGACAATTGACGGCTCGAGGGAGTAGCATAGGGT | |
| | | TTGCAGAATCCCTGCTTCGTCCATTTGACAGGCACATTATGCATCGATGATAAG | |
| | | CTGTCAAACATGAGCAGATCCTCTACGCCGGACGCATCGTGGCCGGCATCACCG | |
| | | GCGCCACAGGTGCGGTTGCTGGCGCCTATATCGCCGACATCACCGATGGGGAAG | |
| | | ATCGGGCTCGCCACTTCGGGCTCATGAGCAAATATTTTATCTGAGGTGCTTCCT | |
| | | CGCTCACTGACTCGCTGCACGAGGCAGACCTCAGCGCTAGCGGAGTGTATACTG | |
| | | GCTTACTATGTTGGCACTGATGAGGGTGTCAGTGAAGTGCTTCATGTGGCAGGA | |
| | | GAAAAAAGGCTGCACCGGTGCGTCAGCAGAATATGTGATACAGGATATATTCCG | |
| | | CTTCCTCGCTCACTGACTCGCTACGCTCGGTCGTTCGACTGCGGCGAGCGGAAA | |
| | | TGGCTTACGAACGGGGCGGAGATTTCCTGGAAGATGCCAGGAAGATACTTAACA | |
| | | GGGAAGTGAGAGGGCCGCGGCAAAGCCGTTTTTCCATAGGCTCCGCCCCCCTGA | |
| | | CAAGCATCACGAAATCTGACGCTCAAATCAGTGGTGGCGAAACCCGACAGGACT | |
| | | ATAAAGATACCAGGCGTTTCCCCTGGCGGCTCCCTCGTGCGCTCTCCTGTTCCT | |
| | | GCCTTTCGGTTTACCGGTGTCATTCCGCTGTTATGGCCGCGTTTGTCTCATTCC | |
| | | ACGCCTGACACTCAGTTCCGGGTAGGCAGTTCGCTCCAAGCTGGACTGTATGCA | |
| | | CGAACCCCCCGTTCAGTCCGACCGCTGCGCCTTATCCGGTAACTATCGTCTTGA | |
| | | GTCCAACCCGGAAAGACATGCAAAAGCACCACTGGCAGCAGCCACTGGTAATTG | |
| | | ATTTAGAGGAGTTAGTCTTGAAGTCATGCGCCGGTTAAGGCTAAACTGAAAGGA | |
| | | CAAGTTTTGGTGACTGCGCTCCTCCAAGCCAGTTACCTCGGTTCAAAGAGTTGG | |
| | | TAGCTCAGAGAACCTTCGAAAAACCGCCCTGCAAGGCGGTTTTTTCGTTTTCAG | |
| | | AGCAAGAGATTACGCGCAGACCAAAACGATCTCAAGAAGATCATCTTATTAAGG | |
| | | GGTCTGACGCTCAGTGGAACGAAAAATCAATCTAAAGTATATATGAGTAAACTT | |
| | | GGTCTGACAGTTACCTTAGAAAAACTCATCGAGCATCAAATGAAACTGCAATTT | |
| | | ATTCATATCAGGATTATCAATACCATATTTTTGAAAAAGCCGTTTCTGTAATGA | |
| | | AGGAGAAAACTCACCGAGGCAGTTCCATAGGATGGCAAGATCCTGGTATCGGTC | |
| | | TGCGATTCCGACTCGTCCAACATCAATACAACCTATTAATTTCCCCTCGTCAAA | |
| | | AATAAGGTTATCAAGTGAGAAATCACCATGAGTGACGACTGAATCCGGTGAGAA | |
| | | TGGCAAAAGCTTATGCATTTCTTTCCAGACTTGTTCAACAGGCCAGCCATTACG | |
| | | CTCGTCATCAAAATCACTCGCATCAACCAAACCGTTATTCATTCGTGATTGCGC | |
| | | CTGAGCGAGACGAAATACGCGATCGCTGTTAAAAGGACAATTACAAACAGGAAT | |
| | | CGAATGCAACCGGCGCAGGAACACTGCCAGCGCATCAACAATATTTTCACCTGA | |
| | | ATCAGGATATTCTTCTAATACCTGGAATGCTGTTTTCCCGGGGATCGCAGTGGT | |
| | | GAGTAACCATGCATCATCAGGAGTACGGATAAAATGCTTGATGGTCGGAAGAGG | |
| | | CATAAATTCCGTCAGCCAGTTTAGTCTGACCATCTCATCTGTAACATCATTGGC | |
| | | AACGCTACCTTTGCCATGTTTCAGAAACAACTCTGGCGCATCGGGCTTCCCATA | |
| | | CAATCGATAGATTGTCGCACCTGATTGCCCGACATTATCGCGAGCCCATTTATA | |
| | | CCCATATAAATCAGCATCCATGTTGGAATTTAATCGCGGCCTCGAGCAAGACGT | |
| | | TTCCCGTTGAATATGGCTCAT | |
| pEG7034 | TruD | 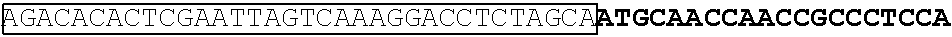 | 669 |
| | | AATTAAGCCCCACTTCCACGTTGAGATAATTGAGCCGAAGCAAGTGTATCTCCT | |
| | | GGGCGAACAGGGCAACCACGCTCTCACCGGGCAGCTCTACTGCCAAATTCTGCC | |
| | | TTTCTTAAACGGCGAATACACCCGAGAACAAATTGTGGAAAAGCTCGATGGGCA | |
| | | GGTCCCGGAGGAATATATCGACTTCGTACTCAGTCGTCTGGTGGAGAAGGGCTA | |
| | | TCTAACTGAGGTGGCTCCAGAACTATCCCTGGAAGTGGCAGCATTTTGGAGCGA | |
| | | ATTGGGAATTGCCCCTTCTGTAGTGGCAGAAGGGCTAAAGCAGCCAGTGACAGT | |
| | | GACAACGGCGGGCAAGGGCATTAGGGAAGGGATAGTGGCTAACCTGGCAGCAGC | |
| | | GCTGGAGGAAGCTGGCATTCAGGTGTCAGACCCAAGGGACCCAAAGGCCCCAAA | |
| | | GGCAGGGGATTCTACTGCCCAGCTTCAGGTGGTGCTGACCGATGACTATTTACA | |
| | | GCCGGAACTTGCAGCGATCAACAAGGAAGCCTTAGAGCGCCAACAACCCTGGTT | |
| | | GCTGGTTAAGCCTGTGGGCAGTATCCTCTGGTTGGGACCGTTGTTCGTTCCTGG | |
| | | GGAAACCGGATGTTGGCACTGTCTTGCTCAACGATTGCAAGGCAACCGGGAAGT | |
| | | TGAAGCATCGGTATTGCAACAAAAGCGAGCGCTGCAGGAGCGCAACGGTCAAAA | |
| | | TAAAAATGGTGCAGTGAGTTGCTTGCCCACAGCACGGGCAACCCTACCTTCTAC | |
| | | TCTACAAACAGGTTTACAGTGGGCTGCCACTGAGATTGCTAAGTGGATGGTCAA | |
| | | GCGGCACCTCAATGCCATAGCACCGGGAACGGCTCGTTTTCCCACTCTAGCTGG | |
| | | CAAGATATTTACATTCAACCAGACGACTCTGGAGTTGAAAGCTCATCCTCTGAG | |
| | | CCGACGACCGCAATGTCCCACCTGTGGCGATCGGGAAACTCTCCAACGGCGCGG | |
| | | GTTTGAACCACTGAAGCTAGAGTCGCGCCCCAAACACTTCACCTCCGATGGCGG | |
| | | TCATCGCGCCATGACCCCAGAACAAACGGTGCAGAAGTACCAACACCTCATCGG | |
| | | GCCCATAACGGGGGTAGTGACGGAACTGGTGCGAATTTCTGACCCTGCCAATCC | |
| | | CTTGGTGCATACCTACCGGGCTGGGCATAGCTTTGGCAGTGCTACGTCTCTGCG | |
| | | GGGGCTGCGCAATGTCCTACGCCACAAGAGTTCTGGTAAAGGCAAGACCGATAG | |
| | | CCAATCTCGGGCCAGCGGACTTTGCGAGGCGATCGAGCGCTATTCGGGCATTTT | |
| | | TCAGGGAGACGAACCCCGCAAGCGGGCAACTTTGGCTGAGTTGGGAGATTTGGC | |
| | | GATTCATCCAGAACAGTGTTTGCACTTTAGCGACAGGCAGTATGACAACCGGGA | |
| | | AAGCTCGAACGAGCGAGCAACAGTGACTCACGACTGGATTCCCCAACGGTTCGA | |
| | | TGCAAGTAAGGCTCACGACTGGACTCCCGTGTGGTCCCTAACGGAGCAAACCCA | |
| | | TAAGTATCTGCCTACAGCCCTGTGCTATTACCGATACCCCTTCCCCCCAGAACA | |
| | | CCGTTTCTGCCGTAGTGACTCCAACGGAAACGCGGCGGGAAATACCCTGGAAGA | |
| | | GGCGATTTTGCAAGGATTTATGGAACTGGTGGAACGGGATAGCGTGTGCCTGTG | |
| | | GTGGTACAATCGCGTTAGCCGTCCGGCTGTGGATTTGAGTAGCTTTGACGAGCC | |
| | | TTATTTTTTGCAGTTGCAGCAGTTCTATCAAACTCAAAATCGCGATCTGTGGGT | |
| | | ACTGGATTTAACAGCAGATTTGGGCATTCCGGCTTTTGTAGGGGTATCGAATCG | |
| | | GAAAGCCGGCAGCTCGGAAAGAATAATTCTCGGCTTTGGAGCGCACCTGGACCC | |
| | | GACAGTTGCCATCCTTCGCGCTCTTACGGAGGTCAACCAAATAGGCTTGGAATT | |
| | | GGATAAAGTTTCTGATGAGAGCCTCAAGAACGATGCCACGGATTGGTTAGTGAA | |
| | | TGCTACATTGGCAGCTAGTCCCTATCTCGTTGCCGATGCTAGCCAACCCCTCAA | |
| | | GACTGCGAAGGATTATCCCCGGCGTTGGAGTGACGATATTTACACCGATGTGAT | |
| | | GACTTGTGTAGAAATAGCCAAGCAAGCAGGTCTAGAGACTTTGGTACTGGATCA | |
| | | GACCAGACCCGACATAGGTTTAAATGTGGTTAAAGTCATTGTGCCAGGAATGCG | |
| | | TTTTTGGTCGCGATTTGGCTCCGGTCGGCTCTATGACGTGCCAGTGAAGTTGGG | |
| | | ATGGCGAGAGCAACCACTTGCTGAGGCACAAATGAACCCTACACCGATGCCATT | |
| | | TTAATAA | |
| pEG7035 | AlbA | 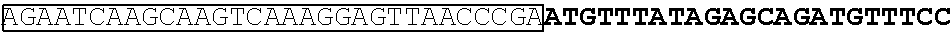 | 670 |
| | | ATTTATTAATGAAAGTGTAAGAGTTCACCAGCTTCCTGAGGGCGGCGTGTTAGA | |
| | | AATCGACTACTTGCGCGATAATGTCTCCATTTCTGACTTTGAGTATTTGGATCT | |
| | | CAACAAAACGGCTTACGAGCTCTGCATGCGCATGGATGGCCAAAAAACAGCTGA | |
| | | GCAGATTTTAGCTGAGCAATGTGCAGTGTATGATGAATCACCGGAAGATCATAA | |
| | | AGATTGGTATTACGACATGCTCAACATGCTCCAGAACAAGCAGGTTATTCAGCT | |
| | | TGGAAACCGGGCCAGCCGCCATACAATCACCACGAGCGGAAGCAATGAATTTCC | |
| | | GATGCCCCTGCACGCCACCTTTGAACTGACGCACCGCTGTAATTTGAAATGCGC | |
| | | CCACTGTTATTTGGAAAGCTCACCTGAAGCGCTCGGCACCGTGTCGATTGAGCA | |
| | | ATTCAAAAAAACGGCTGATATGCTGTTTGATAACGGTGTATTGACATGCGAAAT | |
| | | CACAGGTGGAGAAATTTTTGTCCATCCAAACGCCAATGAGATTCTTGACTATGT | |
| | | GTGTAAAAAGTTCAAAAAAGTCGCTGTCTTAACAAACGGAACACTCATGCGAAA | |
| | | AGAGAGCCTGGAGCTTTTGAAAACTTACAAGCAAAAAATCATCGTCGGCATTTC | |
| | | TCTAGATAGTGTCAATTCCGAGGTCCATGACTCCTTTAGAGGGAGAAAAGGCTC | |
| | | TTTTGCCCAAACTTGTAAAACGATAAAATTGTTGAGTGACCACGGTATATTTGT | |
| | | CAGAGTCGCTATGTCTGTATTCGAAAAAAACATGTGGGAAATCCACGATATGGC | |
| | | CCAAAAGGTTCGGGATCTCGGGGCGAAGGCGTTTTCTTACAATTGGGTTGACGA | |
| | | TTTCGGAAGAGGCAGGGATATTGTCCATCCAACGAAAGACGCCGAGCAGCACCG | |
| | | CAAGTTTATGGAATACGAGCAACATGTGATTGATGAGTTTAAAGATCTGATTCC | |
| | | GATTATTCCCTATGAGAGAAAACGCGCGGCAAATTGCGGCGCTGGCTGGAAGTC | |
| | | CATTGTGATCAGTCCGTTCGGCGAAGTACGTCCTTGCGCCCTCTTTCCAAAGGA | |
| | | ATTTTCATTGGGAAATATTTTTCATGATTCCTATGAAAGCATCTTTAACTCCCC | |
| | | TCTCGTCCATAAACTGTGGCAAGCGCAAGCGCCGCGGTTCAGCGAACATTGCAT | |
| | | GAAAGACAAATGCCCGTTCAGCGGCTATTGCGGAGGCTGTTACTTAAAAGGGCT | |
| | | GAACTCTAACAAATATCACCGGAAAAACATTTGCTCTTGGGCGAAAAATGAACA | |
| | | ATTAGAAGATGTGGTCCAGCTTATTTAGTAA | |
| pEG7037 | MdnC | 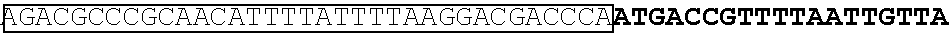 | 671 |
| | | CTTTTAGCCACGATAATGAAAGTATTCCTCTGGTAATCAAAGCCATAGAAGCCA | |
| | | TGGGTAAAAAAGCCTTCCGTTTTGATACTGATCGCTTCCCTACAGAGGTGAAAG | |
| | | TTGATCTTTACTCAGGCGGTCAAAAAGGCGGAATTATTACCGATGGAGAACAAA | |
| | | AATTAGAGCTAAAAGAAGTTTCTTCTGTCTGGTATCGACGCATGAGATACGGAC | |
| | | TAAAATTACCCGATGGGATGGATAGTCAATTTCGCGAAGCTTCTCTTAAGGAAT | |
| | | GTCGGTTAAGTATTCGAGGAATGATTGCTAGTTTATCTGGCTTTCATCTTGATC | |
| | | CAATTGCTAAGGTAGATCATGCTAATCATAAACAATTGCAGTTACAAGTGGCGC | |
| | | AACAATTAGGTTTATTAATTCCGGGGACTTTAACTTCTAATAATCCTGAAGCTG | |
| | | TCAAGCAATTTGCTCGGGAGTTTGAAGCGACGGGAATTGTGACTAAAATGCTTT | |
| | | CTCAATTTGCTATTTATGGAGACAAGCAAGAGGAAATGGTTGTTTTTACCAGTC | |
| | | CTGTTACAAAGGAAGATCTAGATAATTTGGAAGGTTTGCAATTTTGTCCAATGA | |
| | | CTTTTCAGGAAAACATTCCTAAAGCTTTGGAATTACGCATCACTATCGTCGGTG | |
| | | AACAAATATTTACGGCGGCGATTAATTCCCAACAATTAGACGGTGCTATCTACG | |
| | | ATTGGCGAAAAGAGGGACGCGCGCTCCATCAACAATGGCAACCCTACGATTTAC | |
| | | CGAAAACTATTGAAAAACAACTACTAGAATTAGTGAAATATTTCGGTCTTAATT | |
| | | ATGGTGCAATTGATATGATTGTCACACCAGATGAACGTTATATCTTTTTAGAAA | |
| | | TTAATCCCGTTGGCGAGTTTTTCTGGCTAGAACTTTATCCTCCTTATTTTCCTA | |
| | | TCTCCCAGGCGATCGCTGAAATCCTAGTTAACTCATAATAA | |
| pEG7043 | ProcM | 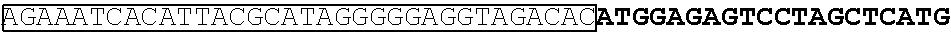 | 672 |
| | | GAAAACATCGTGGCTGGCCGCCATCGCTCCGGATGAACCCCACAAATTCGACCG | |
| | | CCGCTTAGAATGGGACGAGCTTTCAGAGGAGAACTTCTTCGCAGCACTGAACTC | |
| | | AGAACCTGCATCGTTGGAAGAGGATGATCCATGTTTTGAAGAAGCACTGCAAGA | |
| | | CGCCCTGGAGGCCTTGAAGGCAGCATGGGATTTACCCCTTCTTCCCGTCGATAA | |
| | | TAATCTTAATCGTCCCTTCGTAGATGTCTGGTGGCCCATTCGCTGTCACTCTGC | |
| | | GGAGAGCTTGCGTCAAAGCTTCGTCAGTGATAGTGCTGGACTTGCGGACGAGAT | |
| | | TTTTGATCAGCTGGCCGATTCGTTACTGGACCGTCTGTGCGCCCTGGGAGATCA | |
| | | GGTGTTGTGGGAGGCGTTTAACAAGGAGCGTACACCAGGAACGATGTTGTTAGC | |
| | | CCACTTAGGAGCCGCAGGCGACGGCTCCGGACCCCCTGTACGTGAGCATTACGA | |
| | | ACGTTTTATTCAGTCTCACCGCCGTAATGGATTAGCGCCTTTGCTTAAGGAATT | |
| | | CCCTGTACTGGGCCGCCTTATTGGAACAGTTTTGTCCCTTTGGTTCCAAGGGAG | |
| | | CGTGGAAATGCTGCAACGTATCTGCGCTGACCGCACCGTTCTGCAACAGTGTTT | |
| | | CGCTATCCCTTGCGGGCATCACCTGAAAACTGTAAAGCAGGGACTTTCTGATCC | |
| | | ACACCGCGGCGGTCGCGCTGTGGCAGTTTTGGAATTTGCGGACCCAAATTCCAC | |
| | | CGCTAATTCAAGTATGCACGTAGTGTATAAACCGAAGGATATGGCTGTGGATGC | |
| | | AGCTTACCAGGCCACCTTAGCAGATCTTAATACTCATAGCGACCTTTCCCCGTT | |
| | | GCGCACGCTTGCCATTCATAACGGCAACGGATATGGTTACATGGAACATGTGGT | |
| | | TCACCATCTTTGCGCTAACGACAAAGAGCTGACAAATTTCTATTTCAACGCTGG | |
| | | GCGTTTAACCGCGCTTCTGCATCTTCTTGGATGTACTGACTGTCACCATGAAAA | |
| | | TTTGATTGCATGTGGTGATCAATTACTGTTGATCGATACAGAAACATTATTGGA | |
| | | GGCGGATTTACCCGATCACATTTCGGATGCTTCGAGCACCACGGCGCAACCAAA | |
| | | GCCTAGTAGCCTTCAAAAGCAATTTCAGCGTTCTGTTTTGCGTAGCGGGTTACT | |
| | | TCCTCAATGGATGTTCCTGGGGGAGTCGAAGTTGGCCATCGACATCTCGGCTCT | |
| | | GGGAATGTCCCCACCCAATAAGCCTGAGCGTATTGCACTTGGCTGGTTAGGATT | |
| | | CAATTCTGACGGGATGATGCCTGGGCGTGTATCCCAACCAGTTGAGATTCCTAC | |
| | | ATCCTTGCCCGTTGGGATTGGTGAGGTTAATCCCTTTGATCGTTTTTTAGAGGA | |
| | | TTTTTGTGATGGCTTTTCCATGCAATCAGAGGCCCTTATTAAGCTTCGCAACCG | |
| | | TTGGCTGGACGTTAATGGGGTTCTTGCTCATTTCGCGGGTCTGCCCCGCCGTAT | |
| | | CGTTCTTCGCGCGACTCGCGTATACTTCACTATCCAGCGTCAGCAGTTAGAGCC | |
| | | TACGGCACTGCGCTCTCCACTTGCACAGGCCTTGAAACTTGAGCAGCTTACTCG | |
| | | TTCTTTCTTGTTGGCAGAGTCAAAGCCTCTTCACTGGCCCATTTTCGCAGCTGA | |
| | | AGTAAAGCAGATGCAGCACCTTGACATTCCTTTCTTCACACACTTAATCGACGC | |
| | | TGACGCTCTGCAGCTGGGCGGCCTGGAACAAGAATTACCAGGCTTCATCCAGAC | |
| | | TAGTGGCTTGGCAGCTGCTTACGAGCGTTTGCGTAATTTAGATACGGACGAGAT | |
| | | TGCTTTCCAACTTCGTCTGATCCGCGGTGCAGTAGAGGCTCGCGAGTTGCATAC | |
| | | TACGCCGGAGTCGAGCCCGACGTTGCCGCCGCCTGCCACCCCCGAGGCTCTTAT | |
| | | GTCCTCTTCAGCCGAGACTAGTTTAGAAGCTGCTAAGCGCATCGCTCACCGCTT | |
| | | ACTGGAGTTGGCAATTCGTGATTCTCAAGGGCAAGTAGAATGGCTGGGCATGGA | |
| | | TCTGGGGGCAGATGGAGAGAGCTTCTCCTTTGGCCCAGTTGGCTTGAGCCTTTA | |
| | | TGGGGGCTCAATCGGTATCGCTCACCTTCTGCAACGTTTGCAGGCGCAGCAAGT | |
| | | TTCCTTGATGGACGCAGACGCTATCCAAACGGCAATTTTACAGCCCCTTGTGGG | |
| | | ACTGGTTGATCAACCTAGCGACGACGGACGTCGCCGTTGGTGGCGTGATCAGCC | |
| | | GCTGGGCTTAAGTGGATGTGGCGGTACCTTGCTTGCACTTACACTTCAAGGTGA | |
| | | ACAAGCGATGGCTAATTCCCTGCTGGCCGCTGCTTTGCCCCGTTTTATCGAGGC | |
| | | TGATCAGCAACTTGACCTGATTGGTGGCTGCGCTGGACTGATCGGTTCGTTGGT | |
| | | ACAATTAGGTACTGAAAGTGCCTTACAATTAGCTTTGCGTGCGGGCGACCATCT | |
| | | TATTGCGCAACAGAATGAAGAGGGGGCGTGGTCTAGCTCGTCATCACAGCCCGG | |
| | | TTTGTTGGGCTTTAGTCATGGTACTGCAGGTTACGCAGCAGCCTTAGCACACTT | |
| | | ACATGCATTTTCCGCTGATGAGCGTTACCGCACCGCAGCCGCTGCCGCTTTAGC | |
| | | ATACGAACGCGCACGTTTTAATAAAGATGCCGGCAACTGGCCAGACTACCGCTC | |
| | | GATCGGACGTGACTCTGATTCAGATGAACCGTCCTTTATGGCTTCCTGGTGTCA | |
| | | CGGCGCACCCGGCATTGCCCTGGGCCGCGCCTGTTTGTGGGGTACGGCGCTTTG | |
| | | GGACGAAGAATGCACCAAGGAGATCGGAATTGGGTTACAGACCACAGCTGCTGT | |
| | | TTCGTCTGTTAGTACTGACCACCTGTGTTGTGGTTCACTTGGCCTTATGGTATT | |
| | | ATTAGAGATGCTGTCAGCAGGACCCTGGCCCATCGACAATCAATTACGTTCCCA | |
| | | TTGCCAGGACGTAGCATTCCAGTACCGCCTGCAGGCTTTGCAGCGCTGTTCAGC | |
| | | CGAGCCGATTAAGCTTCGTTGCTTCGGTACAAAAGAGGGCCTTTTAGTCCTGCC | |
| | | TGGATTTTTCACTGGCTTATCAGGAATGGGTTTAGCACTGCTTGAGGATGATCC | |
| | | ATCTCGCGCCGTGGTTTCTCAACTGATCAGTGCGGGCTTATGGCCGACAGAGTG | |
| | | ATAA | |
| pEG7047 | MibHS | 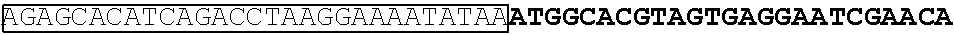 | 673 |
| | | CTCTGGCACGTCTGTTTGACGTGTTGGGTGACGATGCCGCTGCCGCACGTGAAT | |
| | | GGGTAACGGAACCCCATCGTCTGATCGCTAGCAATGAGCGCCTGGGCACAGCTC | |
| | | CGGAAGCCCCGGCGGATGACGATCCGGAGGCCATTCGGACGGTTGGAGTGATCG | |
| | | GAGGGGGCACAGCCGGGTATTTAACGGCGTTGGCTCTGAAGGCTAAACGCCCTT | |
| | | GGTTGGATGTGGCGCTCGTCGAAAGTGCGGATATCCCGATCATTGGGGTAGGAG | |
| | | AGGCGACGGTGTCTTATATGGTGATGTTTCTGCACCATTATCTGGGCATTGATC | |
| | | CGGCGGAGTTTTACCAACATGTGCGCCCTACTTGGAAACTGGGCATCCGTTTTG | |
| | | AATGGGGGTCACGTCCGGAGGGCTTTGTTGCGCCATTCGATTGGGGGACCGGAT | |
| | | CTGTTGGCCTGGTTGGGAGCCTGCGTGAAACGGGCAATGTCAACGAAGCTACGT | |
| | | TACAGGCGATGCTCATGACGGAGGATCGCGTTCCGGTATATCGTGGCGAAGGTG | |
| | | GGCATGTTAGTCTGATGAAATATCTGCCATTCGCATATCATATGGATAACGCTC | |
| | | GCCTGGTTCGCTACCTGACGGAACTCGCCACTCGTCGTGGCGTGCATCATGTCG | |
| | | ATGCGACTGTAGCTGAAGTTCGCCTGGATGGTCCTGACCACGTTGGGGACCTGA | |
| | | TTACTACGGACGGTCGTCGCCTGCACTATGACTTTTACGTCGATTGTACTGGAT | |
| | | TTCGTTCCCTGCTGCTGGAAAAAGCCCTGGGTATCCCGTTCGAATCTTATGCGT | |
| | | CAAGCCTGTTTACCGACGCGGCAATTACCGGTACCCTTGCACATGGGGGTCATC | |
| | | TTAAACCTTACACTACGGCAACTACCATGAATGCGGGCTGGTGTTGGACGATCC | |
| | | CTACTCCTGAGTCCGATCACCTGGGGTACGTTTTCAGTAGTGCCGCGATCGATC | |
| | | CAGACGATGCAGCAGCAGAAATGGCCCGCCGTTTCCCGGGCGTTACCCGCGAAG | |
| | | CATTAGTTCGCTTTCGCTCCGGCCGTCACCGTGAAGCTTGGCGCGGCAATGTCA | |
| | | TCGCGGTAGGAAACAGCTATGCTTTCGTGGAACCTCTGGAGAGTTCGGGACTCC | |
| | | TGATGATTGCTACCGCAGTCCAGATCCTGGTGAGTTTGCTGCCGAGTAGTCGTC | |
| | | GTGACCCGCTGCCTAGCAATGTGGCGAATCAGGCGTTAGCTCACCGGTGGGACG | |
| | | CGATTCGTTGGTTTCTGAGTATTCATTACCGTTTCAACGGCCGCCTCGATACTC | |
| | | CGTTCTGGAAGGAAGCCCGTGCCGAAACAGATATTAGCGGTATTGAACCGTTGC | |
| | | TTCGTCTGTTCAGTGCCGGTGCCCCTCTGACCGGTCGCGATAGCTTTGCGCGCT | |
| | | ATTTGGCCGACGGAGCAGCCCCGTTGTTCTATGGCCTGGAGGGTGTTGATACCT | |
| | | TACTGCTGGGACAGGAAGTGCCTGCGCGTCTGTTACCACCGCGTGAATCTCCTG | |
| | | AGCAGTGGCGTGCCCGTGCTGCAGCAGCCCGCTCATTAGCCTCGCGTGGCTTAC | |
| | | GTCAGAGCGAAGCTCTGGATGCTTACGCTGCGGACCCCTGTCTCAATGCGGAAC | |
| | | TGCTGTCTGATAGCGACTCATGGGCGGGTGAACGCGTCGCGGTACGTGCAGGTC | |
| | | 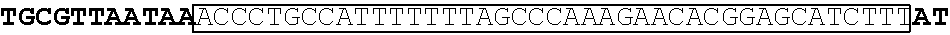 | |
| | | GACGACTGGCACCACGGTAGCGCATGCTGTAGAACCAGACGGTTTCCGCGCCGT | |
| | | GATGGCCACACTGCCGGCCGCTGTGGCGATCGTTACGGCAGCTGCGGCAGATGG | |
| | | GCGCCCGTGGGGTATGACCTGCAGTTCGGTTTGCTCAGTGACCTTGACCCCGCC | |
| | | GACCCTTCTGGTCTGCCTTCGGACGGCGTCCCCGACTCTGGCCGCAGTCGTGTC | |
| | | AGGTCGTGCATTTAGCGTGAACCTTCTGTGTGCGCGGGCCTATCCTGTGGCGGA | |
| | | ATTGTTTGCATCTGCGGCAGCAGACCGGTTTGATCGCGTTCGTTGGCGTCGCCC | |
| | | GCCGGGTACAGGCGGTCCACATCTTGCCGATGATGCACGTGCAGTGTTAGACTG | |
| | | TCGCCTGAGCGAAAGCGCAGAAGTAGGCGACCATGTGGTCGTATTTGGCCAAGT | |
| | | CCGGGCGATTCGTCGCCTGAGTGATGAACCACCACTGATGTATGGTTATCGTCG | |
| | | TTACGCACCTTGGCCGGCAGATCGTGGTCCGGGTGCGGCAGGCGGCTAATAA | |
| pEG7048 | MibD | 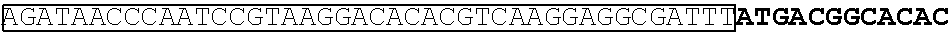 | 674 |
| | | AGCGACGCAGGAGGTGACCCACGCCCGCCTGAACGCTTACTGTTGGGGGTGTCA | |
| | | GGAAGTGTCGCTGCACTGAACTTACCGGCGTACATTTATGCCTTTCGGGCAGCC | |
| | | GGTGTGGCACGTCTTGCGGTCGTGCTGACACCAGCGGCTGAAGGGTTCCTTCCA | |
| | | GCGGGTGCGTTACGCCCGATTGTGGATGCCGTTCATACGGAACATGACCAAGGC | |
| | | AAAGGTCACGTAGCGCTGTCACGCTGGGCGCAACACTTACTCGTGCTGCCGGCA | |
| | | ACAGCGAATTTGCTTGGCTGTGCAGCGTCAGGACTTGCGCCGAACTTTTTAGCG | |
| | | ACCGTTCTGCTCGCGGCAGATTGCCCAATCACATTCGTCCCGGCGATGAATCCG | |
| | | GTCATGTGGCGTAAACCAGCCGTACGCCGGAACGTTGCAACCTTACGCGCAGAT | |
| | | GGTCATCACGTGGTGGATCCTCTGCCGGGCGCTGTGTACGAAGCTGCCTCACGT | |
| | | TCTATCGTGGAAGGTCTTGCTATGCCGCGCCCTGAAGCGTTAGTCCGTTTACTG | |
| | | GGTGGCGGTGATGACGGTTCTCCAGCAGGACCGGCAGGTCCGGTTGGACGCGCA | |
| | | GAGCATGTTGGGGCTGTTGAGGCTGTTGAAGCCGTGGAAGCAGTTGAGGCCGTT | |
| | | GAGGCTGCGGAAGCACTTGCGTAATAA | |
| pEG7056 | PlpXY | 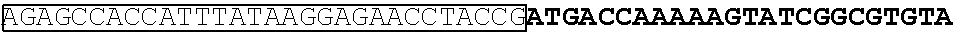 | 675 |
| | | TCCTACGCAGTGTGGGAAATCACCCTGAAATGCAATCTGGCATGCTCTCATTGT | |
| | | GGCAGCCGCGCCGGCCAAGCCCGTACGAAAGAGCTGAGTACCGAAGAAGCGTTC | |
| | | AACCTGGTCCGCCAGCTGGCCGACGTGGGCATTAAGGAAGTCACCCTGATCGGT | |
| | | GGTGAAGCCTTTATGCGTTCGGATTGGCTGGAAATCGCGAAAGCCGTCACTGAA | |
| | | GCCGGCATGATCTGTGGCATGACCACAGGGGGCTTCGGGGTCAGTCTGGAAACG | |
| | | GCGCGTAAAATGAAAGAAGCGGGCATTAAAACGGTGAGCGTTAGCATTGACGGT | |
| | | GGTATTCCTGAAACCCACGACCGCCAGCGCGGTAAAAAGGGTGCGTGGCATAGT | |
| | | GCATTCCGGACTATGAGCCATCTGAAAGAAGTCGGGATCTACTTCGGTTGCAAC | |
| | | ACTCAAATCAATCGTTTATCGGCGTCAGAATTCCCGATTATCTATGAACGTATT | |
| | | CGCGATGCTGGGGCACGTGCGTGGCAAATTCAGCTGACGGTTCCGATGGGCAAC | |
| | | GCCGCGGATAACGCAGATATGCTGCTGCAACCGTATGAATTGCTCGACATCTAT | |
| | | CCGATGTTAGCCCGCGTTGCCAAACGTGCGAAACAGGAAGGCGTGCGTATTCAG | |
| | | GCAGGTAACAACATCGGGTACTATGGACCGTATGAGCGTCTGCTGCGTGGCAGC | |
| | | GACGAATGGACGTTTTGGCAAGGATGTGGTGCGGGCCTTAACACCCTCGGCATC | |
| | | GAAGCCGACGGCAAAATCAAAGGCTGTCCATCCCTGCCGACCGCCGCGTACACC | |
| | | GGCGGTAACATTCGCGATCGCCCGCTGCGGGAAATCGTCGAACAGACCGAAGAA | |
| | | CTGAAATTTAACTTAAAAGCTGGTACAGAACAAGGTACGGACCATATGTGGGGC | |
| | | TTTTGTAAAACCTGCGAATTCGCGGAACTCTGTCGCGGCGGATGCAGCTGGACT | |
| | | GCGCATGTGTTCTTTGACCGGCGCGGCAATAATCCGTACTGCCACCATCGGGCT | |
| | | CTGAAACAAGCCCAAAAAGACATTCGCGAACGCTTTTATTTAAAAGTGAAAGCA | |
| | | AAGGGCAACCCGTTCGACAATGGTGAATTTGTTATCATTGAAGAACCTTTTAAC | |
| | | GCTCCGTTACCCGAGAATGACCTGCTGCACTTTAACAGTGATCACATTCAATGG | |
| | | 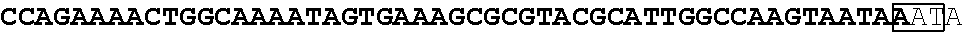 | |
| | | 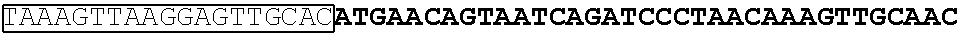 | |
| | | CGCGGCACAGAAATCTGACGACAGCAGCAGCGTATTACCGCGCCAGGGGTGGCA | |
| | | AGACAAACAAGCCTTTATTAAGGCACTCATTAAAGCCAAACAGTCTCTCGAAAT | |
| | | TGCCGAAATTAGCAACTTTTTAACC | |
| pEG7058 | PbtO | 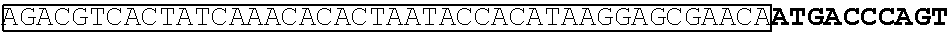 | 676 |
| | | ATCCCCTGTCGCGTCCAGAACCGCTGGGCGTGCACCCAGATTATCGTCGCCTGC | |
| | | GTGAGACTTGCCCGGTTGCACGTGTGGGTAGCCCGTATGGCCCAGCGTGGCTTG | |
| | | TCACCCGTTACGCCGATGTGGCCGCAGTTCTGACCGATGCCCGTTTTAGTCGTG | |
| | | CAGCCGCTCCGGAAGATGATGGTGGCATCCTGCTGAACACCGATCCGCCGGAAC | |
| | | ATGATCGTCTGCGTAAACTGATTGTAGCACACACAGGCACCGCTCGCGTGGAAC | |
| | | GGCTGCGTCCGCGTGCTGAAGAGATCGCTGTTGCGTTAGCGCGCCGTATCCCGG | |
| | | GCGAAGGCGAATTCATTAGTGCATTTGCCGAGCCCTTCAGCCATCGCGTTTTGT | |
| | | CTTTATTTGTTGGCCATCTTGTTGGGTTACCAGCGCAGGACCTGGGCCCCTTAG | |
| | | CGACCGTAGTGACTCTGGCACCCGTTCCCGACCGCGAACGTGGCGCGGCATTTG | |
| | | CAGAGCTGTGTCGTCGGCTGGGTCGTCAGGTGGATCGCGAAACGCTTGCAGTAG | |
| | | TTTTAAACGTGGTCTTTGGCGGACATGCGGCTGTAGTGGCCGCGCTGGGTTATT | |
| | | GCCTGTTAGCTGCATTAGATGCGCCACTGCCACGTCTGGCCGGTGACCCAGAGG | |
| | | GCATTGCCGAACTGGTGGAAGAAACCCTTCGTTTGGCTCCACCGGGAGATCGTA | |
| | | CACTGTTGCGTCGTACTACAGAACCTGTGGAACTTGGCGGTCGCACATTACCAG | |
| | | CGGGTGCGCTTGTAATCCCGTCCATTGCAGCCGCAAACCGTGATCCGGATCGCC | |
| | | CTGTGGGCCGTCGTATGCCACGTCATCTTGCATTTGGACGTGGAGCGCATGCCT | |
| | | GTTTAGGCATGGCGCTGGCGCGCATGGAACTCCAGGCAGCACTGAAAGCGTTAG | |
| | | CGGAACACGCGCCAGACGTACGGTTGCCGGCTGGTACAGGCGCGCTGGTCCGCA | |
| | | CACACGAAGAACTCTCGGTGAGCCCGCTCGCAGGAATCCCAATTCAACGCTAAT | |
| | | AA | |
| pEG7059 | PbtM1 | 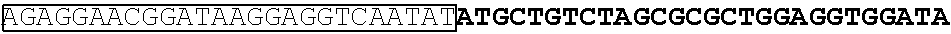 | 677 |
| | | TCGATGAAGCTGCGGTGGCGGCGGACTTACGCGAATTGGCCGCAGCTCTGGATC | |
| | | GCAGTGGTTATGGTGAAATCCTCACCTGTTTTCTGCCTCAGAAGGCACAGGCGC | |
| | | ATATCTGGGCTCAGACCGCTGCAAAAATTGATGGGCCGTTGCGTACCCTGATGG | |
| | | AATTATTCCTTCTGGGTCGGGCGGTTCCCCAGGATGATCTCCCGCCTCGCATCG | |
| | | CGGCCGTGATTCCCGGTTTAGTTAGCGCAGGTCTGGTTAAGACTGGACAGGGCG | |
| | | CGGTTTGGCTGCCGAACTTGATTCTGCTGCGTCCTATGGGCCAGTGGTTATGGT | |
| | | GTCAGCGGCCTCACCCCTCACCGACCATGTACTTTGGTGACGATAGCCTGGCGC | |
| | | TGGTTCACCGGATGGTAACATATCGTGGCGGCCGTGCCCTGGATTTATGTGCAG | |
| | | GTCCGGGTGTTCAGGCCCTTACCGCAGCCCTCCGCTCAGAGCACGTTACCGCGG | |
| | | TTGAGATCAATCCGGTCGCGGCAGCCCTTTGCCGCACCAACATTGCCATGAACG | |
| | | GTCTGTCCGACCGCATGGAGGTTCGCCTGGGCTCACTGTACGACGTCGTGCGCG | |
| | | GTGAGGTTTTTGATGATATTGTATCAAACCCGCCGCTGCTGCCTGTTCCGGAGG | |
| | | ATGTGCAATTCGCCTTTGTGGGAGATGGCGGACGCGATGGTTTCGATATTTCTT | |
| | | GGACGATTCTGGATGGCCTGCCTGAACATCTGTCCGACCGTGGTGCGTGTCGCA | |
| | | TCGTTGGTTGTGTTCTGTCCGATGGCTATGTGCCTGTTGTGATGGAAGGCTTGG | |
| | | GAGAATGGGCCGCTAAACACGATTTCGACGTGCTTCTTACAGTGACCGCACATG | |
| | | TCGAGGCGCATAAAGATAGTAGTTTTCTGCGTTCAATGAGCCTGATGAGTTCGG | |
| | | CGATCTCAGGCCGCCCAGCGGAGGAGCTGCAAGAACGGTACGCAGCTGATTATG | |
| | | CCGAACTGGGCGGTTCCCACGTTGCGTTCTATGAACTGTGTGCCCGCCGTGGTG | |
| | | GGGGTTCTGCACGTCTGGCCGACGTGAGCGCTACAAAACGCAGTGCGGAAGTGT | |
| | | GGTTTGTTTAATAA | |
| pEG7060 | PaaA | 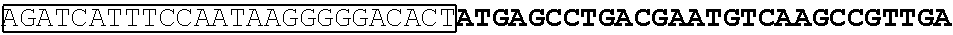 | 678 |
| | | TTAAAGAATCCCACCACATCATTTTAGCTGACGATGGTGACATTTGCATTGGGG | |
| | | AAATTCCGGGGGTGTCTCAGGTAATCAATGACCCGCCGTCGTGGGTTCGTCCTG | |
| | | CCCTGGCAAAGATGGATGGCAAGCGTACTGTCCCCCGTATTTTCAAAGAACTGG | |
| | | TCAGTGAAGGCGTACAGATCGAATCCGAACATCTGGAAGGCCTGGTAGCCGGGC | |
| | | TTGCCGAACGCAAACTTCTCCAGGATAACAGTTTCTTTTCCAAGGTGTTAAGCG | |
| | | GTGAAGAAGTGGAGCGCTATAACCGCCAGATTCTGCAGTTCAGCCTTATCGATG | |
| | | CGGATAACCAGCACCCTTTCGTTTACCAAGAGCGGCTGAAACAGTCTAAAGTCG | |
| | | CTATCTTCGGTATGGGTGGCTGGGGCACGTGGTGTGCATTGCAGCTGGCCATGT | |
| | | CAGGCATTGGTACACTGCGGCTGATCGACGGCGATGATGTGGAACTGTCGAACA | |
| | | TTAACCGCCAAGTTCTGTATCGCACGGATGATGTAGGTAAAAACAAAGTTGATG | |
| | | CCGCCAAAGACACTATCCTGGCATACAACGAAAACGTGCATGTTGAAACCTTCT | |
| | | TTGAATTCGCCAGCCCGGACCGTGCCCGGCTTGAAGAACTTGTGGGTGATTCTA | |
| | | CCTTTATTATCCTGGCTTGGGCCGCGTTGGGTTACTACCGTAAAGATACGGCAG | |
| | | AGGAAATTATCCATTCGATTGCGAAAGATAAAGCGATCCCTGTAATTGAACTCG | |
| | | GCGGTGATCCTTTGGAAATCTCTGTCGGTCCTATTTACCTGAATGATGGCGTAC | |
| | | ACAGCGGCTTCGACGAGGTGAAAAATTCCGTTAAAGATAAATACTACGACAGCA | |
| | | ACAGCGATATCCGCAAATTTCAAGAGGCGCGGTTGAAACACAGCTTCATCGATG | |
| | | GCGATCGTAAAGTGAACGCGTGGCAATCAGCGCCCAGCCTGAGTATTATGGCTG | |
| | | GTATCGTAACGGATCAGGTTGTGAAAACCATTACCGGGTACGACAAGCCACATC | |
| | | TCGTTGGCAAGAAATTTATCTTGAGTCTGCAAGATTTCCGCAGCCGCGAGGAGG | |
| | | AGATCTTTAAATAATAA | |
| pEG7066 | CinX | 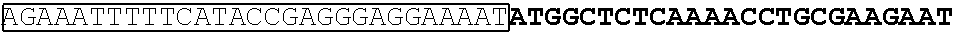 | 679 |
| | | TTCTGCGCGATGCGTTAGATCCGGATCGCTTCGGCCGCGAGATGAAGGCAGTAA | |
| | | CAGAAATTCCCGAGATCGTTAAACTCGGCCATCGTCATGGTTATGGATTTACTG | |
| | | CCGAAGAATTTCTGACCAAAGCTATGAGTTTTGGTGCTCCGCCGGCAGGAGCAG | |
| | | CAGCACCTGGCGAATCAGCCAGCGTTCCTGGCCAGAACGGTTCCTCCCCCGGAC | |
| | | ACGCTGCGCGTGCAGCTATGGCTGGTCCAGAAGCAGGGGCCACCAGCTTTGCCC | |
| | | ACTATGAATACCGTCTGGATGAGCTGCCGGAATTCGCCCCCGTTGTGGCCGAGC | |
| | | TTCCGAAACTGAAAGTCATGCCGCCTTCCGTGGGACCTGATCGGTTTGCAGCAC | |
| | | GCTACCGTGATGAAGATATGCGCACAATTTCAATGAGTCCGGCGGATCCGGCTT | |
| | | ACCAGGCTTGGCACCAGGAACTGGCGGGTCGTGGTTGGCGCGATGCAGAAGATA | |
| | | CGGCTGCTGCTCCAGATGCCCCACGGCGCGATTTTCATCTGCTGAACCTCGATG | |
| | | AGCATGTAGATTACCCAGGTTATGAAGAATATTTTGCGGCCAAGACCCGTGTCG | |
| | | TCGCGGCACTCGAAAACCTGTTTGGTGGTGACGTGCGTTGCTCAGGCTCTATGT | |
| | | GGTATCCGCCGTCGAGCTATCGCTTATGGCATACAAATGCCGATCAACCGGGGT | |
| | | GGCGTATGTACCTGGTAGATGTAGATCGCCCATTCGCGGACCCCGACCGTACCT | |
| | | CCTTCTTTCGCTACCTGCATCCACGTACCCGTGAAATCGTCACGCTGCGCGAAA | |
| | | GCCCTCGTATTGTCCGTTTCTTTAAAGTCGAACAGGATCCCGAGAAGCTGTTCT | |
| | | GGCACTGTATCGCGAACCCCACCGATCGCCATCGCTGGTCGTTTGGTTACGTTG | |
| | | TTCCGGAAAACTGGATGGACGCCCTCCGTCACCATGGCTAATAA | |
| pEG7067 | CapBC | 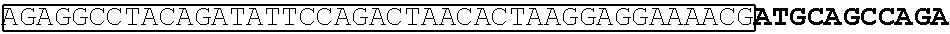 | 680 |
| | | CCTGGAGGTTGTTGATGTTCGTCGCGGCGAGTCGTTCAAGGCATGGTCGCATGG | |
| | | GTACCCATATCGCACTGTTCGCTGGCACTTCCATCCTGAGTTTGAAGTACATCT | |
| | | GATCGTGGAAACCACCGGCCAGATGTTTGTGGGTGATTATGTCGGAGGCTTTGG | |
| | | TCCGGGTAATCTGGTCCTGATGGGTCCCAATCTGCCTCATAATTGGGTGTCTGA | |
| | | CGTTCCTGAGGGTAAAACCGTTGCAGAGCGTAACCTTGTTGTTCAATTTGGGCA | |
| | | AGCGTTCGTTTCCCGTTGCGAGGATTCCTTAACGGAGTGGCGTCACGTGGAAAC | |
| | | GTTACTGGCGGATGCGCGGCGTGGCGTGCAATTTGGGCCGCGCACCTCTGAGGC | |
| | | CATTAAACCTCTGTTCGCGGAACTGATTCACGCGCGCGGCCTGCGTCGCATTGT | |
| | | GCTGTTTCTGTCTATGCTGCAAATCCTCGTCGATGCAACGGATCGCGAACTGCT | |
| | | GGCATCTCCAGCTTATCAGGCGGATCCTTCGACATTTGCAAGCACGCGCATTAA | |
| | | TCATGCGCTGGCCTACATTGGAAAGAATCTGGCGAACGAGCTTCGTGAAACAGA | |
| | | TTTAGCACGGCTGGCCGGACAGTCTGTTTCCGCCTTCTCTCATTATTTTCGTCG | |
| | | TCATACCGGCCTGCCTTTCGTGCAGTACGTTAATCGCATGCGTATCAACCTGGC | |
| | | CTGTCAGCTTCTGATGGACGGGGACGCATCGGTGACAGATATTTGTTTCCGTAG | |
| | | CGGTTTTAACAACCTGTCCAATTTTAACCGTCAGTTTCTGGCAGTGAAAGGTAT | |
| | | GTCACCCAGTCGGTTCCGTCGCTACCAGGCTCTCAACGACGCGTCACGTGATGC | |
| | | GAGTGAAGCGGCTGCAAAACGCGGCGCAGGTATTGCAGGTGCACCGGCAATCGT | |
| | | TCCAGCGGCTCAAGCACGTGGCGAGGCACGCCCAATTCCTGAAGTGCTGCTTAG | |
| | | 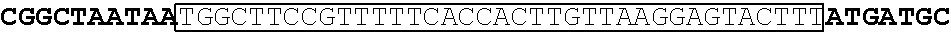 | |
| | | TGACGGCGAGCTCCACACCGGCATCCGGTAATCCAGCTGCCCGTGCATTGCGCG | |
| | | CCGCTGCCTTTGCACTGGCCTTAGGCGGAGCATGCGTTGCGCATGCCGCACCTC | |
| | | TGCGGATTGGCATGACATTCCAAGAATTGAATAACCCGTATTTTGTGACCATGC | |
| | | AGAAAGCACTGAACGAAGCCGCGGCGAGCATTGGCGCGCAAGTGATTGTAACAG | |
| | | ACGCACATCACGACGTGTCAAAACAGGTATCAGACGTTGAGGATATGCTGCAGA | |
| | | AGAAAATTGATATTTTACTGGTGAATCCAACCGACTCCACGGGCATCCAGAGTG | |
| | | CGATTGTTTCCGCAAAGAAGGCTGGCGCCGTGGTCGTGGCGGTCGATGCCAATG | |
| | | CCAATGGCCCGGTGGATTCCTTCGTAGGGTCCAAGAATTTTGATGCCGGCGCTA | |
| | | TGTCATGCGAGTACCTTGCGAAAGCGATCAACGGCGGCGGCGAAGTGGCCATTC | |
| | | TGGATGGCATCCCGGTCGTCCCAATCCTGGAACGTGTCCGCGGCTGCCGCGCGG | |
| | | CACTGGCCAAATTCCCGAATGTGAAAATTGTCGACGTTCAGAATGGAAAACAGG | |
| | | AACGTGCGACAGCGTTAACGGTAACCGAGAATATGATCCAGGCGCACCCGAAAC | |
| | | TGAAAGGTGTGTTTAGTGTAAACGACGGCGGGTCAATGGGCGCTTTGAGCGCCA | |
| | | TTGAAGCGAGCGGCAAAGATATCCGCCTCACGTCCGTAGATGGTGCCCCAGAGG | |
| | | CGGTGGCGGCGATTCAAAAGCCGAACTCCAAATTTATTGAAACAAGCGCTCAAT | |
| | | TTCCGCGCGACCAGATCCGTTTAGCGATTGGTATTGGCCTGGCCAAGAAATGGG | |
| | | GCGCGAACGTGCCAAAAGCGATTCCAGTCGACGTGAAACTGATTGACAAAGGGA | |
| | | ACGCGAAAACCTTTAGTTGGTAATAA | |
| pEG7068 | LasBCD | 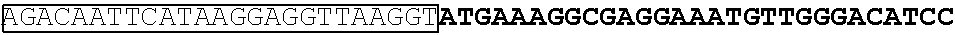 | 681 |
| | | ACAGACCGGTTTTGTTGTACTGCCAGACAACGATGCCACCGGCGACGTGACGGG | |
| | | CCGCCTGTTACCTTGGGGTGATGTAGTTACAGTGTATCCGTCTGGCCGTCCATG | |
| | | GATCATCGGCAACTGCTGGGATCGCCCAGTCCTCGTCCATGATGGCGTGATCGT | |
| | | CTTGGGTCATACCAGCGTCACGCGTGATCAAATTGCCCGTCATGGGAACGATCC | |
| | | GCATCGCTTACTGGACGAGGCCGACGGCGCATTTCATGCGGCGGTCCTGATCGG | |
| | | ACACGAAGTTCATGTTCGCGGCTCCGCCTACGGTGTCTGTCGTCTGTATACATG | |
| | | CGTTGTTGACGGTGTGACCTTAGTGAGTGATCGTACAGACGTCCTGCAGCGTCT | |
| | | GGCAGGTACTGATGTGGACGTCGACGTGCTGGCTGGCCACTTGTTAGAGCCGAT | |
| | | CCCGCACTGGTTAGGCGAACAACCGTTATTGACGTCCGTGGAGCCCGTGCCACC | |
| | | GACACATCACGTTATTTTAACTCCGGACGCACGTAGTCGTTTACGGCCATCACG | |
| | | TCGTCGTCGGCCTGAACCGTCGCTGGGTTTGCGGGACGGTGCGGAACTTGTCCG | |
| | | GGAGCGTCTGGCCGCAGCTGTGGCTACCCGTGTGGACAGTCCAGCGTTAATTAC | |
| | | CAGTGAACTGAGTGGCGGCTATGATTCCACTAGTGTGTCATACTTGGCAGCGCG | |
| | | CGGTAAAGCCGAGGTGGTGCTGGTCACGGCCGCGGGACGTGACAGCACAAGCGA | |
| | | GGATCTGTGGTGGGCTGAACGCGCAGCCGCAGGGCTCCCGGAACTCGATCACGT | |
| | | AGTGTTACCTGCGGATGAATTACCGTTTACGTACGCCGGCCTGACGGAGCCTGG | |
| | | TGCACTTTTGGATGAACCGTGTACGGCTGTTGCCGGCCGTGAGCGTGTACTGGC | |
| | | GCTGGTACGTAAAGCCGCGGCCCGCGGCTCTACACTTCATCTGACTGGCCATGG | |
| | | TGGCGATCACCTGTTTACTTCACTGCCGACACCGTTTCATGACCTGTTTCGTAC | |
| | | GCGTCCAGTCGCCGCGCTCCGCCAGTTGCGTGCATTTGGCGCGTTGGCTGCGTG | |
| | | GCCGACCCGTAAGCTGATGCGCGAACTCGCGGACCGCCGCGATCATAGCACCTG | |
| | | GTGGCGCGCGCACGCACGTCCTCAGAATGGCCAGCCGGATCCGCACAGCCCCAT | |
| | | GTTAGGCTGGGCAATTCCCCCGACTGTCCCGGCGTGGGTTACTGCTGACGGCGT | |
| | | GCGCGCGATCGAACTTGGGATTTTAGAAATGGCAGAACGCGCGGAGCCCCTTGG | |
| | | TCATGCGCGCGGAGAACACGCTGAGCTGGATTCAATCTTTGAAGGGGCGCGTAT | |
| | | GGCCCGTGGCCTCAATCGTATGGCTACGCATGCCGGAGTCCCGCTTGCAGCCCC | |
| | | GTTCCATGACGATCGGGTCGTGGAAGCGTGTCTGTCGATCCGGCCGGAGGAACG | |
| | | catttctgcatggcagtacaaacccttactgaacgccgcaatgcagggtgtggt | |
| | | GCCGAGCACCGTTCTTGATCGTAGCGCTAAAGATGACGGGAGTATTGATGTGGC | |
| | | CTATGGGCTGCAGGAACACCGTGATGAACTGGTAGCGCTGTGGGAATCATCACG | |
| | | TCTGGCGGAAACCGGTCTGATTGATGCGGGTATGCTGCGGCGTTTATGCGCGCA | |
| | | GCCGTCCTCCCACGAGCTCGAGCATGGATCCTTGTACGCTACTATCGCTTGTGA | |
| | | 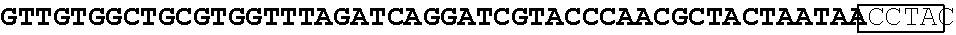 | |
| | | 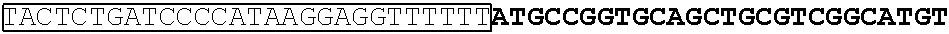 | |
| | | GTCTTTTACGGCTACGGAATACGGCGGCGTGCTGCTGGATGAAACCAAAGGCGC | |
| | | ATACTGGCGTCTGAACACCACAGGCGCCGAAGTTGTTCGCGCCATGGGGGAAGC | |
| | | CGAGCGGGATGAGATTGTACGGCATGTGGTGGCGACCTTCGATGTTGATGCGCA | |
| | | AACCGCAGCCCAGGATGTCGATGTCCTGCTGGCAGAACTTCGTGATGCCGGCCT | |
| | | 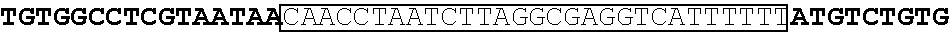 | |
| | | AATATGGCTCTCCGTGGCCATGGTATGTCCGGTCGCCGTCGTCGCTTAGATGCC | |
| | | ACGCGTGCTCGCCTGGCCGTTGTGGTTGCCCGTGTCCTGAATCTCTTACCGCCG | |
| | | CGCTTAATCCGTCGTTGTTTGCGTGTACTGAGTCGCGGAGCCCGCCCTGCCTCG | |
| | | ATTGAGGCAGCAGAAGCTGCTCGTCGTACTGTGGTTGCGGTGAGTCCAGCTGCC | |
| | | GCCGGTGCGTACGGCTGTTTAATCCGCAGCATTGCCACCACCCTGGTTCTTCGT | |
| | | TCACGCGGGCAATGGCCAACCTGGTGTGTTGGTGTACGTGCGGAGCCTCCTTTT | |
| | | GGTGCCCATGCCTGGATTGAAGCAGAGGAGCGGCTGGTGGATGAACCTGGTACT | |
| | | ATGCATACTTACCGTCGTCTTATCACCGTTGGTCCACTGTCTCGCAAAGTTCGT | |
| | | TAATAA | |
| pEG7069 | LasF | 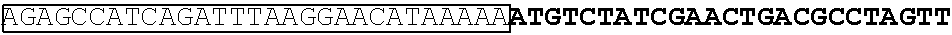 | 682 |
| | | TGGCCGATCTGGTCGATCCACTTCCAGGTCACGCACTGCGCGCTGCGGCGACAT | |
| | | TACGTCTGGCAGATCTGATTGCGGCTGGTGCAGATACTGCACCGGCATTAGCAG | |
| | | CGGCGGCACGCATTGATGCTGACGCGATCGCGCGTCTTATGCGGTATCTGTGCA | |
| | | GTCGCGGGATTTTTCAAGCACATGAAGGCCGGTACGCGTTGACTGAATTTAGCG | |
| | | AATTGCTGCTGGATGAAGATCCATCTGGCCTGCGTAAAACCTTAGATCAGGATA | |
| | | GCTATGGGGATCGTTTCGACCGCGCGGTTGCGGAACTGGTGGACGTTGTACGGT | |
| | | CCGGTGAACCTTCTTATCCTCGCCTTTACGGCTCGACGGTTTATGATGACCTGG | |
| | | CAGCCGATCCTGCCCTCGGCGAGGTGTTCGCGGATGTTCGTGGCTTGCACTCCG | |
| | | CAGGGTATGGGGAAGATGTCGCGGCAGTGGCGGGTTGGTCCTCATGCCTGCGCG | |
| | | TTGTCGATCTGGGTGGAGGGACTGGCTCCGTCCTGCTTGCTGTGTTAGAGCGTC | |
| | | ACCCGTCCCTGTCAGGCGCAGTACTGGATCTGCCATACGTCGCCCCGCAGGCAA | |
| | | AGAAAGCTCTGCAGGCCTCAGCGTTTGCCCAACGTTGTGAATTTATCAAAGGGA | |
| | | GCTTCTTCGATCCGTTACCTCCGGCAGACCGTTACCTGTTGTGTAACGTGCTGT | |
| | | TCAACTGGGATGACGCGCAAGCAGGCGCTATTTTGGCACGCTGTGCGCAGGCGG | |
| | | GCCCTGTGGCCGGAGTAGTGGTAGCCGAACGTTTGATCGATCCGGATGCGGAAG | |
| | | TGGAACTCGTAGCAGCTCAAGATCTGCGTCTGTTGGCTGTTTGCGGCGGTCGGC | |
| | | AGCGTGGCACCGCTGAATTCGAAGCGCTTGGGGCAGCCCATGGCCTGGCGTTAA | |
| | | CCAGCGTTACCCTCACGGCATCTGGTATGAGCCTGCTCCGTTTCGATGTGTGTC | |
| | | GTGCCGGGAGTGCTGGCGGGGAAGTTGTGGAAAAATCTTAATAA | |
| pEG7070 | AlbsBC | 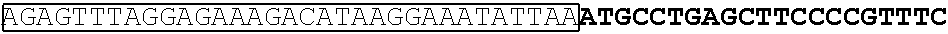 | 683 |
| | | GCGACGGCCCCTCGTCACGTGCGTGCCCTGGATTTCGGTCATGTTCTGGTCCTG | |
| | | ATCGATTACCGTTCCAATCACGTCCAGTGCCTGCTTCCGGCAGCCGCAGCCCAT | |
| | | TGGACAGCCACAGCGCGTACCGGCCGCTTGGACACCATGCCGGCAGCGCTGGCC | |
| | | ACCCAGTTACTGACATCGGCGTTATTAGTACCGCGGCCGACCGCAACACCGTGG | |
| | | ACGGCACCTGTAGCGGCACCACCTGCTCCACCGTCATGGGGTGGATCCGAGCAT | |
| | | CCTGCCGGGACATCACGCCCTCGGGCACGTCATCGGCACTCAACCACGGCTGCG | |
| | | GCGGCGCTGGCATGTGTGCTGGCGATTAAGGCAGCAGGCCCAACCCGCTATGCT | |
| | | ATGCAGCGCTTGACCACGGTCGTGAAGGCAGCCGCTTCTACGTGCCGTCGCCCG | |
| | | GCAACGCCAGCACAAGCGACGGCTGCTGCGCTTGCGGTCCGTCAGGCATGCTGG | |
| | | TACTCGCCAGCGCGTACAGCCTGTCTGGAAGAATCCGCCGCGACTGTCATTTTA | |
| | | CTCGCTACCCGGCGTTTGAGTTCGACATGGTGCCATGGAGTAGCTCCCGATCCG | |
| | | ATTCGCCTCCATGCCTGGGTGGAAACTGAGGATGGGACACCTGTAGCAGAGCCA | |
| | | GCCTCGACCCTTGCGTACACCCCGGCCTTAACCATTGGAGGCCACCATCAACAC | |
| | | 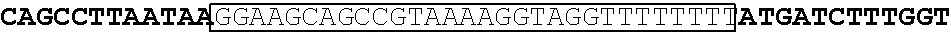 | |
| | | GGATTTTCGACGACCCGTGAAGTTCGTCAACGCCCTGGTAATGCCGAGTTTATT | |
| | | GCTACGGACTCGCCTATTTGGCGCCTCGGTCGTAGTCCAGCTCGTTGCGTGGCT | |
| | | GCGGACCATGGACAGCGTCGCCTGGTAGTGTTGGGAGAATGCGGGGCAACGGAT | |
| | | GGCGAATTATCTCGCCTGGCGACCGCGGGGCTGCCCACGGATATTACCTGGCGC | |
| | | TGGCCAGGCGTGTACGTGGTGGTCGAAGAACAACCGGAACGTACGGTGCTGCAC | |
| | | ACTGATCCAGCAGCTGCACTCCCGGTATACGCAACCCCTTGGCAAGGCGGCTGG | |
| | | GCATGGTCAACCAGCGCGCGCATCCTGGCACGTTTAACAGAAGCTCCAATTGAT | |
| | | GGTCAACGCCTGGCATGTTCAGTGCTGGCCCCGTCTGTTCCGGCTCTGAGCGGT | |
| | | ACCCGCACATTCTTTGCGGGTATCGAACAATTGGCCCTGGGTTCGCGTATTGAA | |
| | | CTGCCGGTGGATGGGTCCCGTCTGCGTGTTACGGTACGTTGGCGCCCGGATCCA | |
| | | GTCCCGGGAGAACCATATCATCGCTTGCGCACAGCGTTGACCGAGGCGGTCGCC | |
| | | CTGCGTGTCAACCGCGCACCAGACCTGTCATGCGACCTCTCGGGCGGCCTCGAT | |
| | | TCCACGTCACTGGCAGTCCTGGCGGCTGTGTGCTTACCGGAGTCCCACCATCTG | |
| | | AATGCTATCACGATTCATCCGGAGGGCGATGAAAGTGGCGCGGACTTACGGTAT | |
| | | GCGCGCTTGGCAGCTGCGCACCACGGGCGTATTCGCCACCACCTTCTCCCCCTT | |
| | | GCGGCAGAACACCTGCCGTATACTGAAATTACGGCGGTGCCCCCTACCACCGAA | |
| | | CCGGCACCTTCAACATTAACGCGTGCACGCCTCGCGTGGCAGTTAGATTGGATG | |
| | | CGCCAGCACTTAGGCAGCCGCACCCATATGACTGGCGATGGAGGCGACAGCGTA | |
| | | CTGTTCCAACCGCCGGCACATCTGGCGGATCTCCTGCGGCATCGGCAGTGGCGT | |
| | | CGGACTTTGTCGGAAAGTTTGGGATGGGCACGCCTTCGCCATACGTCTGTTTTA | |
| | | CCCTTACTGCGTGGAGCAGCAACTCTTGCACGTACATCACGTCGGTCGGGCCTC | |
| | | CAGGATCTCGCACGCGCATTGGCGGGTGCAGGTCAGCAGGGCGATGGTCGTGGC | |
| | | AATGTGAGCTGGTTCGCACCATTACCGCTGCCTGGCTGGGCGACCCCAACCGCT | |
| | | CGTCGCTTACTGCTTGATGCAGCCGATGAAGCTATCTCGACCGCGGATCCGTTA | |
| | | CCGGGACTGGATACGTCGCTGCGCGTACTGATCGATGAAATTCGCGAAGTCGCC | |
| | | CGCACGGCAGCGGCAGATGCCGAACTGGCGGATGCTCACGGAACGACTCTGCAT | |
| | | AACCCATTTCTCGATCCGCGCACTATTGATGCAGTCCTGCGCACGCCAATCGCA | |
| | | CATCGCCCGGCGGTCCACTCGTATAAGCCAGCGCTGGGGCATGCAATGCAGGAT | |
| | | TTGCTCCCGGGTGCAGTCGCTCGGCGCTCAACTAAAGGCTCTTTTAACGCCGAT | |
| | | CATTATGCGGGGATGCGTGCAAATCTGCCAGCATTGACAGCGCTGGCAGATGGC | |
| | | CACCTGGCCGACCTGGGTTTGTTGGAGCCGACGCGCTTCCGCAGTCATCTTCGC | |
| | | CAAGCCGCCGCGGGCATTCCGATGCCGCTTGCGGCGATCGAACAGGCGCTGTCT | |
| | | GCCGAAGCATGGTGTCATGCACATCACGCCACCCCAAGCCCTGCCTGGACAACG | |
| | | CAGCCACCGGAACACCCGCATGCCTAATAA | |
| pEG7071 | AlbsT | 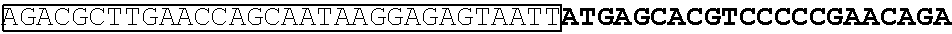 | 684 |
| | | CCCTCTGGATCTCAACTGATACCTGTGGTCTGGGGCCGTATCGCGCTGACTTGG | |
| | | TGGATACCTATTGGCAGTGGGAACAAGACCCAACATTGCTTGTAGGCTACGGTC | |
| | | GTCAGTCACCGCAGTCACTGGAGGCCCGCACGGAAGGTATGGCCCACCAATTGC | |
| | | GTGGCGATAACATCCGTTTCACTATCTATGATCTGTGCAGCAGTACACCTACCC | |
| | | CGGCGGGCGTGGCAACGCTGCTGCCCGATCATAGCGTCCGTACTGCCGAGTATG | |
| | | TTATTATGCTTGCGCCTGAAGCACGTGGGCGTGGCTTAGGAACCACCGCCACGC | |
| | | AGCTGACGTTAGATTATGCGTTTCACATCACCAATCTGCGGATGGTCTGGTTGA | |
| | | AAGTACTGGCGCCGAACACCGCGGGCATCCGTGCGTATGAGAAAGCTGGCTTTC | |
| | | GTACAGTTGGAGCGCTTCGCGAAGCCGGCTATTGGCTGGGGAAGGTCTGCGATG | |
| | | AGGTACTGATGGATGCCTTAGCGAAAGACTTCACGGGTCCAAGTGCAGTCCACG | |
| | | CAGCATTAACTGGCGCCAGCGGTCGCCAGCTGCGCCGTGCACCTTAATAA | |
| pEG7073 | McbCD | 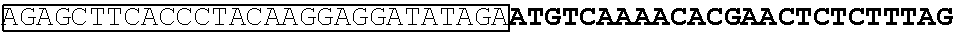 | 685 |
| | | TGGAAGTAACGCATTACACAGATCCTGAAGTTCTGGCCATTGTTAAAGATTTTC | |
| | | ATGTCAGAGGTAACTTTGCTTCCCTCCCCGAATTTGCTGAACGAACTTTCGTGT | |
| | | CCGCGGTACCTCTTGCCCATCTGGAGAAATTTGAAAATAAAGAAGTTCTCTTCA | |
| | | GGCCAGGTTTCAGCTCCGTAATAAACATATCCTCATCACATAATTTTAGTCGTG | |
| | | AAAGGCTCCCATCAGGAATAAACTTTTGCGACAAAAATAAACTTTCCATTCGTA | |
| | | CTATTGAAAAGTTATTAGTCAATGCATTCAGCTCACCTGATCCTGGCTCTGTAA | |
| | | GGCGGCCTTATCCTTCTGGGGGGGCATTGTACCCGATTGAAGTTTTTTTATGCA | |
| | | GATTATCTGAAAATACAGAAAACTGGCAGGCAGGAACTAATGTTTATCACTACC | |
| | | TGCCGCTAAGTCAGGCACTAGAACCTGTTGCTACATGTAATACTCAGTCACTCT | |
| | | ACCGAAGCCTGTCCGGTGGGGATTCGGAACGTCTTGGTAAACCCCATTTTGCTC | |
| | | TCGTCTATTGCATTATTTTTGAAAAAGCTTTGTTCAAATATCGCTACAGAGGAT | |
| | | ACCGGATGGCCTTAATGGAAACAGGTTCGATGTATCAGAACGCAGTATTGGTTG | |
| | | CAGATCAAATAGGACTGAAAAACCGGGTATGGGCGGGATATACCGATTCATACG | |
| | | TAGCAAAAACAATGAATCTGGATCAGAGGACTGTAGCGCCACTGATCGTTCAGT | |
| | | TTTTTGGAGATGTAAACGATGATAAATGTCTACAGTAACCTTATGTCCGCATGG | |
| | | CCGGCCACAATGGCCATGAGTCCAAAACTGAACAGAAATATGCCAACGTTTTCT | |
| | | CAGATATGGGACTATGAGCGTATTACACCAGCCAGCGCGGCCGGTGAAACTCTG | |
| | | AAGTCAATTCAGGGGGCAATAGGTGAATATTTTGAACGCCGTCATTTTTTTAAT | |
| | | GAGATAGTCACCGGTGGTCAGAAAACATTATATGAGATGATGCCTCCATCTGCT | |
| | | GCAAAGGCTTTTACCGAAGCATTTTTTCAGATCTCATCACTGACCCGCGATGAA | |
| | | ATCATAACCCATAAATTTAAAACGGTCAGAGCCTTTAATCTGTTTAGCCTTGAA | |
| | | CAACAAGAAATACCTGCAGTCATAATTGCACTCGACAATATAACCGCTGCAGAT | |
| | | GATCTGAAATTTTATCCTGACAGAGATACATGCGGATGTAGCTTTCATGGTAGT | |
| | | TTGAACGATGCCATAGAAGGTTCCTTGTGTGAATTTATGGAGAGACAGTCCCTC | |
| | | CTTCTTTACTGGTTACAGGGAAAAGCCAATACTGAAATATCCAGTGAAATAGTA | |
| | | ACAGGCATAAATCATATAGATGAGATTTTACTGGCTCTCAGGTCAGAAGGAGAT | |
| | | ATCAGGATTTTCGATATCACCCTGCCCGGAGCTCCTGGACACGCAGTACTAACC | |
| | | CTGTATGGCACAAAAAACAAAATCAGTCGAATAAAATACAGTACCGGATTATCC | |
| | | TATGCTAATAGTCTGAAAAAAGCACTTTGTAAATCCGTAGTGGAATTGTGGCAA | |
| | | TCGTATATATGCCTGCACAACTTTCTTATTGGCGGTTATACTGATGATGACATT | |
| | | ATTGATAGTTACCAGCGTCACTTTATGTCATGCAACAAGTACGAGTCGTTTACG | |
| | | GATTTGTGTGAAAATACGGTACTACTGTCTGATGATGTCAAGTTAACGTTTGAG | |
| | | GAAAATATTACGTCAGACACAAATTTATTAAACTATCTTCAACAAATTTCTGAT | |
| | | AATATTTTTGTTTACTATGCCAGGGAAAGAGTAAGTAACAGCCTTGTCTGGTAC | |
| | | ACAAAAATAGTAAGCCCTGATTTTTTCCTTCATATGAATAACTCAGGTGCAATA | |
| | | AACATTAATAATAAAATTTACCATACCGGGGACGGTATTAAAGTCAGAGAATCA | |
| | | AAGATGGTACCATTCCCATAATAA | |
| pEG7074 | MibO | 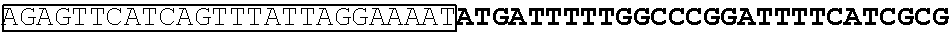 | 686 |
| | | ATCCGTATCCAGTGTATCGTCGTCTGCGTGATGAGGCTCCGTGCCACCATGAAC | |
| | | CAGCGTTAGGTCTGTATGCGTTGAGCCGCTACGAGGACGTTCTGGCTGCCCTTC | |
| | | GTCAGCCCACCGTGTTCAGCTCAGCAGCGCGTGCGGTAGCCTCCAGTGCAGCGG | |
| | | GAGCAGGTCCATACCGCGGTGCCGACACCGTTAGTCCGGAGCGGGAAACTGCGG | |
| | | CTGAAGGGCCCGCCCGTAGCCTGTTGTTCCTGGATCCGCCAGAGCACCAGGTGC | |
| | | TGCGTCAGGCGGTGTCCCGTGGCTTTACGCCGCAGGCAGTATTGCGCCTTGAGC | |
| | | CGGCCGTCCGCGACATTGCGGCGGGTCTTGCTGATCGTATCCCCGATCGCGGTG | |
| | | GTGGCGAGTTCGTTACCGAATTTGCGGCTCCGCTGGCAATCGCAGTGATTCTGC | |
| | | GGTTACTTGGTGTACCGGAAGCAGATCGTGCCCGCGTAAGCGAACTTTTATCGG | |
| | | CATCAGCCCTGTCGGGGGCGGAAGCAGAACTGCGCTCCTATTGGCTGGGCCTTT | |
| | | CGGCACTCCTCCGCGATCGTGAAGATGCAGGCGAAGGTGACGGAGAGGATCGTG | |
| | | GTGTGGTGGCGGCTCTGGTCCGTCCTGATGCTGGACTGCGCGACGCGGATGTTG | |
| | | CCGCAGGACCTGCCGTGCGTGCACCGCTGACGGATGAGCAGGTTGCAGCATTCT | |
| | | GCGCCTTAGTGGGGCAAGCCGGCACTGAAAGTGTGGCAATGGCGCTCTCCAACG | |
| | | CATTGGTCCTGTTCGGGCGTCACCATGACCAGTGGCGCACACTGTGTGCGCGTC | |
| | | CGGATGCGATTCCAGCAGCATTCGAAGAGGTCCTCCGCTATTGGGCACCTACGC | |
| | | AGCATCAAGGTCGGACGTTAACCGCGGCGGTACGTTTACATGGCCGTCTGCTGC | |
| | | CGGCCGGTGCGCATGTACTGCTGCTGACCGGTTCAGCCGGCCGGGATGAACGTG | |
| | | CGTACCCAGACCCCGATGTATTTGACATCGGTCGCTTCCACCCGGATCGTCGTC | |
| | | CGTCGACCGCGCTGGGTTTTGGTCTGGGCGCACACTTTTGTTTAGGCGCTGCTC | |
| | | TCGCTCGTCTGCAGGCACGCGTAGCGCTGCGCGAACTGACACGCCGGTTCCCGC | |
| | | GTTATCGTACGGACGAGGAACGCACTGTGCGTTCGGAAGTGATGAACGGGTTCG | |
| | | GCCACAGCCGTGTACCATTTTCCACGTAATAA | |
| pEG7076 | LtM1 | 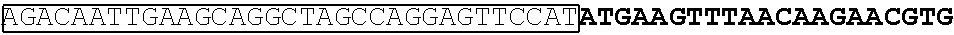 | 687 |
| | | TTCCCAGAGATCAATGAAACGGATTTCGATAACAATATCAAGCCCCTGCTGGAT | |
| | | GAACTGGAATCTCGTATTACCATTCCGCAGGAGGAACTGAGCTTTTCAAGCATT | |
| | | AACGATGATTTATTTCGCGAGTTAACCCGCAACGAGGAGTACCCTTACCAGAGC | |
| | | ATTTGTACGATCGTTGCAAACATCGTGATGGATGACGGCAGTGAGATTTGGCGC | |
| | | AAAGATATTTTTGTTGATTCCAATAGTGTGCGCGAAGCCGTATGCGACATTCTG | |
| | | AGCCAAACGTTATTCCTCTATTTCATCCGCTGCTTCTCCGAACAAATTAAAGAC | |
| | | ATTCGCAAAACTGATGAGGATAAAGAGTCCACCTACAACCGCTACATTAACCTC | |
| | | CTGTTCAGCTCCAACTTCAAAATCTTCTCCGACGAATACCCTGTCCTGTGGTAT | |
| | | CGGACCATTCGCATCATCAAAAATCGCTGGTATTCTATCAAGAAATCGTTACTG | |
| | | CTGACTCAAAAACACCGTGTGGAGATCGATAAGCAGTTGGACATCCCGCACAAG | |
| | | ATGAAGATTAAAGGCCTGAAAATCGGGGGAGACACGCATAACGGCGGTGCCACA | |
| | | GTGACCACGATCTTCTTTGAGAAAGGGTATAAACTGATTTATAAGCCGCGGAGC | |
| | | ACATCCGGCGAATTCTCGTACAAGAAATTTATCGAAAAGATTAACCCGTACCTG | |
| | | AAGAAAGACATGGGAGCGATTAAAGCGATCGATTTCGGTGAATACGGCTTTTCT | |
| | | GAGTATATTGAGTGTAACACGGATGAAGAGGACATGAAACAGGTCGGTCAGCTT | |
| | | GCATTTTTCATGTACCTGTTGAATGCATCAGATATGCATTATAGCAATGTCATT | |
| | | TGGACCAAACAGGGCCCTGTGCCGATTGATTTAGAAACCTTGTTCCAGCCGGAT | |
| | | CGTATTCGCAAAGGCCTGAAGCAGTCGGAAACTAACGCGTACCACAAAATGGAG | |
| | | AAAAGTGTATACGGAACGGGAATTATTCCAATTTCCCTGAGCGTTAAAGGCAAA | |
| | | AAGGGTGAGGTCGACGTCGGCTTTAGTGGAATCCGTGATGAGCGCTCTAGTTCG | |
| | | CCGTTTCGCGTTCTGGAAATTTTGGATGGGTTTTCGAGCGACATCAAAATCGTG | |
| | | TGGAAAAAGCAGCAGAAGTCTAGCTCCAGCAAAAACAATCTGATTGTCGATCAC | |
| | | AAAAAGGAGCGCGAAATCCTTCAGCGTGCCCAGTCCGTCGTAGAAGGTTTCCAG | |
| | | GAAACCTCTAAAATCTTCATGAAACATCGTGAGGAATTCATCTCCATTATCTTA | |
| | | GACTCATTCGAGAACATCAAAATTCGCTACATCCATAACATGACGTTTCGCTAC | |
| | | GAACAGTTGCTGCGCACTCTGACGGATGCCGAGCCGGCCCAGAAGATTGAGTTA | |
| | | GACCGTCTGCTGCTGAGTCGTACCGGAATTCTGTCCATCTCGTCTAGTCCCTAC | |
| | | ATCTCGCTCTCCGAATGTCAACAGATGTGGCAGGGTGACGTGCCGTACTTCTAC | |
| | | TCGAAGTTTTCGAGCAAAAGTATCTTTGATACCAATGGCTTCGTTGATGAAATC | |
| | | GAGCTGACGCCCCGCCAGGCATTTATCATCAAAGCCGAAAGTATCACCAACGAT | |
| | | GAAGTCGATTTTCAGTCCAAGATCATTAAACTGGCGTTCATGGCACGCTTAAGT | |
| | | GACCCGCACACAACCAACGACAACAAACTGAATAAAAAGGTGATTATCGAAAGC | |
| | | AACCAGCAGAGCAACAGCAGTGAATCAGGTAACAAAGCCATTTTGTTCCTGAGC | |
| | | GATCTGCTGAAAAATAACGTACTGGAAGATCGTTATAGTCATCTGCCGAAAACT | |
| | | TGGATTGGCCCTGTAGCACGTGATGGCGGTTTGGGTTGGGCGCCGGGCGTGCTG | |
| | | GGATACGATCTGTACTCGGGCCGTACAGGACCTGCGTTAGCATTGGCTGCGGCC | |
| | | GGGCGCGTTTTGAAAGATAAAGACAGTATCGAACTTAGCGCCGACATTTTTAAT | |
| | | AAATCGTCCCAGATTCTGCAGGAAAAGACTTACGACTTTCGTAACCTGTTCGCA | |
| | | TCAGGTATCGGCGGTTTTAGCGGGATTACCGGTCTGTTTTGGGCGCTGAACGCG | |
| | | GCAGGGAATATTCTGAACAATGATGACTGGATTAAAACCTCGAATCAGAGTATG | |
| | | CTGCTGCTGAATGAGAACATGCTGAAAGTGGACAAAAATTTCTTTGACCTGATT | |
| | | AGCGGCAACTCGGGAGCGATCGGTATGATGTACCTGACCAATCCAAATTTCTAT | |
| | | TTGTCTCGCTCGAAAATTAACGACATTCTGCTGACCACGGACTGCTTGATTACT | |
| | | GAAATGGAAAAAGACGAAACGAGCGGACTGGCCCATGGCGTGTCTCAGATCCTG | |
| | | TGGTTCCTTAGCATTATGATGCAACGTCAGCCCTCAAGTGAAATCAAAATCCGC | |
| | | GCGACGATTGTCGACAACATCATCAAGAAGAAGTATACGAATTCCTATGGCGAA | |
| | | ATCGAATGCTACTATCCGACTGATGGGCACTCCAAATCCACCTCGTGGTGCAAC | |
| | | GGGACAAGTGGGATTCTGGTCGCCTATATTGAGGGGTATAAAGCTAATATCGTG | |
| | | GACAAATCCTCGGTGTATCATATTATTAATCAGATCAACGTCGAACAACTTCAG | |
| | | CATGATAACATTCCGATCATGTGCCATGGTAGCCTTGGTGTGTATGAATCGCTT | |
| | | AAATATGCGTCAAAGTACTTTGAAATCGAAACCAAGTACCTTCTGGATGTGATG | |
| | | CGCAATGGCGGCTGCTCCTCCCAAGAAGTATTAAAGTACTATGGCAAGGGTAAC | |
| | | GGCCGTTACCCGCTGTCACCAGGTTTAATGGCGGGTCAGTCGGGCGCGTTGCTG | |
| | | CACTGTTGCAAACTGGAGGATAACGATATCAGCGTGAGCCCCATTTCACTGATG | |
| | | ACGTAATAA | |
| pEG7077 | LtnM2 | 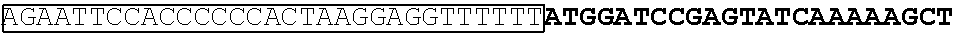 | 688 |
| | | CGTGGATTCTATCATCGAATTCTACAAAAAGGACATCTACCTGGCATACAAAGA | |
| | | GCTGGAACGCGAAATCAAAAACATCGATAAGACCATCTACAACACTTCAAATGA | |
| | | CGAGATCTTGCGGATTTTTAAAGAGAGCCTGATCAGCATCATCACCGATGATAT | |
| | | TTACCGCCTCTCGATTAAAACCTTCATCTATGAGTTTCACAAGTTTCGTATCGA | |
| | | TAACGGGTTTCCGGCTGTCAAAGATAGCGAAAGCGCCTTCAATTATTACATCAG | |
| | | TACCTTTGACGTGAAAACGATCGCTCGCTGGTTTGAGAAATTCCCAATGCTGGA | |
| | | ATCCATCATCTCCAGTAGCATCAAAAACGATTGCACATTTATGGTGGATGTATG | |
| | | TGTCAATTTCATCTTAGACCTGTCGGAATGCGAGAAGATTAATCTGATCTCAGA | |
| | | GGATAGCCGGCTCATCACGATCTCATCCAGCAACTCTGACCCGCACAACGGTGG | |
| | | CACGCGTGTCTTGTTCTTTCGTTTCCACAACGGTGATACCATTCTTTACAAACC | |
| | | CCGCAGCCTGACCGTGGACAAGCTGATCTCTAATATTTTCGAAGAGGTATTCGA | |
| | | ATTCGATGCGACGAACTCGAAAAATCCTATTCCCAAGGTGCTGGATCGGGGTAC | |
| | | CTATGGCTGGCAGGAATTCATTGAGAAGAAATCGATCTCTTCCTCAGAGATTAA | |
| | | GCAGGCCTACTATAACCTGGGTATCTTTAGCAGTATCTTTACAGTGTTAGGGTC | |
| | | TACTGATATCCACGATGAAAACTTGATTTTTAAAGGTACGACCCCGTATTTCAT | |
| | | CGATCTGGAAACAGCCCTCTCTCCGCGTATCCGGTATGAAGGTAATGAGGAAAA | |
| | | CCTGTTCTATCGGATGAGCTCATCGTTGTTCACTTCTATCGTGGGGACGACTAT | |
| | | TATTCCTGCAAAACTTGCTGTCCATTCCCAGGAAATTATGATCGGCGCAATTAA | |
| | | CACCCCTGCGAAACAGAAAACCAAGAAGGATGGCTTTAACATCATCAACTTCGG | |
| | | CACGGATGCCGTCGATATCGCAAAACAGAATATTGAGGTGGAGCGTATTGCTAA | |
| | | CCCTATGCGCATTAAAAATAACATCGTGAACGATCCGCTGCCGTACCAGAACAT | |
| | | CTTTACGCGCGGCTTCAAAGAGGGGATCAAATCCATCATCCTGAAGAAAGGCTC | |
| | | GATCATTTCCATTCTGAACAACTTCAACAGCCCGATTCGTTACATCATGCGGCC | |
| | | GACGGCAAAATATTATTTGATTCTGGATGCCGCGGTATTTCCCGAAAACCTGTA | |
| | | TTCGGAACAGACACTGAACAAAACCCTGAATTACTTAAAGCCGCCAAAAATCGT | |
| | | GGAAAATTCCCTGATTTCTAAACAGCTCTTTCTTGCCGAAAAACGCATTCTGTC | |
| | | CGAAGGCGATATTCCGAGCTTCTATGTGCTGGGCAAAGAGAAAAATATCCGTGC | |
| | | GCAGAACTTCATTAGCGAACAGATCTTCGAGGAAACCGCGGTCGATAACGCGAT | |
| | | TCAAATTCTGGAATCCATTTCGCAAGACTGGGTGAATTTTAATGAGCGCCTGAT | |
| | | TGCGGAGGGCTTCTCCTATATTCGTGAACAGAGTCGTGGCTATCTGTCCAGTGA | |
| | | TTTTGAGAACTCTGATATTTTCAAAAGCTCACTGACCGAAACAAAGAAGTCCGG | |
| | | TTATACCGCAATGCTGAAAACAATTATCTCCATGTCGGTCAAGACCTCGGAAAA | |
| | | CAAAAAGATCGGTTGGCTGCCAGGCATTTATGATGATTATCCGATCAGCTATAT | |
| | | GAGTGCCGCGTTTTGTTCGTTCCATGATTCCGGCGGTATCATCACTTTGCTTGA | |
| | | ACACCACTTTGGGCACTGCTCCCCCGAATATAACGAGATGAAGCGCGGGCTGCT | |
| | | GGAACTGGGCAAAATGTTGAAAATTAACAATAGTAACCTGAGCATCATCTCCGG | |
| | | CTCAGAGTCTCTGGAATTTCTGTATACGCACCGCGAAGTCGAATGCCTGGAACT | |
| | | GGAATACATTTTAAACAATTCAGCGGAAATCATGGGCGACGTGTTCCTGGGGAA | |
| | | ATTAGGCCTTTATCTTATCCTGGCGAGCTACCTGAAAACAGACCTGAAAATTTT | |
| | | CCAAGATTTCAGTATCATCTGCCAGAAAAACCTCGAGTTTAAAAAGTTCGGGAT | |
| | | CGCGCACGGTGAATTAGGGTATCTGTGGACCATCTTCCGTATTCAAAACAAACT | |
| | | GAAGAACAAAAATGCGTGTCTGAGCATCTATCATGAAGTGTTGAACATTTATAA | |
| | | AGGTAAGCGCATTGAATCCGTGGGATGGTGCAACGGTTTATCGGGTATTCTGAT | |
| | | GATTTTGTCAGAAATGAGCACCGTATTAGAGAAAAATCAAGACTATCTGTTCAA | |
| | | GCTGGCAAATCTGAGCACTAAACTGAATGAGGAATCCGTTGACCTGAGTGTGTG | |
| | | CCACGGCGCCAGCGGGGTGCTTCAAACACTGCTTTTCGTCTATAGCAACACGAA | |
| | | CGATAAACGTTATCTCAGCCTGGCCAATAAGTATTGGAAGAAAGTGCTGGATAA | |
| | | CAGCATTAAGTACGGTTTCTACAATGGAGAACGCGATAAGGATTATCTGTTGGG | |
| | | ATATTTCCAGGGTTGGTCAGGCTTCACGGACAGCGCACTCCTGCTGGATAAATA | |
| | | CAATAACAATGAGCAAGTGTGGATTCCGATCAACCTGAGCTCCGATATCTATCA | |
| | | GCATAATCTGAACAACTGCAAAGAGAAGAATTATGAGGGCGATGGCTGCCATAA | |
| | | ATCTTAATAA | |
| pEG7078 | CrnM | 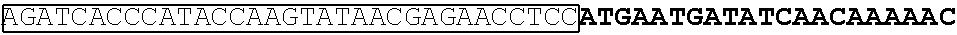 | 689 |
| | | AAAACTAAAACCATTAACGAAAAGATTAAAATTTTCACCAAAGAAGAGGTGATT | |
| | | GATATCAGTTACTTTGAAGAATGGCGCAGCGTTCGTACTCTGCTTAACGAAAAC | |
| | | TACTTTAAAATTATGCTCGAGGAAATGAATATTTCCAAAAACCAATTTTCGTAT | |
| | | GCGCTGCAACCGTTAAACGACGAGTTCAAACTGCATACTAACGTTAAAAATGAA | |
| | | GAATGGATCAAATGCTTTAATCGCGTCATTAACAATTTTAACTATAAAAATATT | |
| | | AACTATAAAGTTGGTTTGTACCTGCCTATTCAGCCTTTCTCCGTTTATTTACAG | |
| | | GAGAAACTGAAAGAGATCCTGAAGAAGCTGAACAACATTAAGATTAATGATAAA | |
| | | ATTATCGACGCCTTTATCGAAGCTCACCTGATCGAAATGTTCGACCTCGTCGGT | |
| | | AAAGTAATCGCCCTTAAATTTGAAGATTATAAACAGATCAACTTCCTGAAAAAC | |
| | | ACAAATAATGGCACCCGCTTGGAGGAATTCTTGCGTAGCACCTTTTATTCTCGG | |
| | | AAGTCATTTCTGAAACTGTTTAACGAGTTTCCGGTACTCGCGCGGGTTTGCACC | |
| | | GTACGTACGATCTATTTGATCAATAACTTTAGTGCTATCATCCAGAACATCAAT | |
| | | AGCGACTACCTGGAAATCCAGGAATTTCTGAACGTCGATTTCCTGAACTTGACA | |
| | | AACATCACTCTTTCGACGGGTGATTCCCACGAACAGGGTAAAAGTGTGTCCATC | |
| | | CTCTATTTTGATGAAAAAAAGCTGATTTATAAACCGAAAAATCTGAAGATTTCA | |
| | | GAAATTTTCGAGAGCTTCATCGACTGGTACACCAACGTCTCTAACCATAAGCTG | |
| | | CTCGACCTGAAAATCCCGAAAGGAATTTTTAAAGACGATTACACTTATAACGAA | |
| | | TTTATTGAGCCAAACTACTGCGAGAATAAGCGCGAAATTGAAAATTACTATAAC | |
| | | CGTTATGGGTACCTGATCGCAATCTGTTATCTGTTCAACCTGAATGACCTGCAT | |
| | | GTAGAAAATGTGATCGCCCATGGCGAGTACCCGGTTATTGTTGATATTGAAACG | |
| | | AGCTTTCAAGTCCCTGTGCAAATGGAGGACGATACTTTATATGTGAAGCTGTTG | |
| | | CGCGAGCTGGAATTGGAAAGCGTTTCATCGTCGTTTCTGTTACCTACCAATCTG | |
| | | TCGTTTGGTATGGACGATAAAGTGGACCTGTCCGCGCTGAGCGGAACCATGGTC | |
| | | GAGCTGAATCAGCAAATTCTGGCGCCTGTCAACATTAATATGGACAACTTTCAT | |
| | | TACGAGAAATCACCGAGCTATTTTCCAGGCGGAAACAATATCCCTAAAAACAAC | |
| | | AAATCAGTGACTGTTGATTATAAAAAATACTTGCTCAATATTGTGACTGGTTTC | |
| | | GACGAATTTATGAAGTATACCCAAGAAAATCAGCTGGAATTTATTGAGTTCCTG | |
| | | AAAAAATTCTCAGATAAAAAAATCCGGGTGCTGGTGAAGGGTACGGAAAAATAT | |
| | | GCGTCCATGATTCGCTACAGCAACCATCCGAACTACAACAAAGAAATGAAATAT | |
| | | CGCGAGCGTCTCATGATGAACTTGTGGGCGTACCCTTACAAAGACAAGCGTATT | |
| | | GTTAATAGCGAAGTACAGGACCTGTTATTTAACGATATCCCGATCTTTTACTCC | |
| | | TTTCCAAATAGCCGTGACCTCATTGATAGTCGCGGCTTGGTGTATAAAGATTAC | |
| | | CTTCCTGTGACAGGACTGCAGAAAGCAATTGATCGCGTGAAAGATACCTCGGTA | |
| | | AAAAGCTTGTTCGACCAGAAGCTGATTCTTCAGAGTAGCTTAGGTCTGTGGGAT | |
| | | GAGATTCTCAACAAGCCGGTCCAGAAAAAGGAACTGCTCTTTGAAAAGCAGAAC | |
| | | TTTAACTATGTGAAAGAGGCGATCAATATTGCGGAATTGCTGATTGGCTATTTA | |
| | | ATCGAAACGGACGACCAGAGCACCATGCTGAGCATTGATTGTTCTGAAGATAAA | |
| | | CACTGGAAGATTGTTCCTTTAGACGAATCCCTGTATGGTGGGCTGTCCGGCATT | |
| | | GCATTATTTTTTCTCGATATTTATAAAATTACCAAAGATGAAAAATATTTTAAT | |
| | | TACTATGATAAAATCATTTCCACGGCCATTAAACAATGTAAAGCGACCATCTTC | |
| | | TCGTCAAGCTTCACGGGTTGGCTGAGTCCCATTTATCCGTTGATTCTGGAAAAG | |
| | | AAATACTTTGGTACCATGAAAGATAAGAAATTCTTTGACTACACGATGGAAAAG | |
| | | CTGTCGAATATGACTGAAGAACAAATTAACAACATGGATGGTATGGACTATATC | |
| | | AGTGGCAAGGCGGGTATTGTCAAACTGCTGATTAGCGCGTACCGGGAATCGAAG | |
| | | AACAATGAAAACATCGGACTGGCCCTGAGTAAATTCAGCAACGATCTGATTCAA | |
| | | AATATTGGCACCGGCAAAGTCAGTGAATTACAAAACGTGGGCCTGGCGCACGGC | |
| | | ATTTCTGGTATTATGGTCGTAGTAGCCTCACTGGACACGTTTAAAAGTGAATAT | |
| | | ATTCGCGAGCAGCTGGCAATTGAATATGAGATGTTCTGTTTGCGTGAAGATTCA | |
| | | TACAAATGGTGTTGGGGCATCTCTGGAATGATTCAAGCCCGTCTCGAAATTCTG | |
| | | AAACTGAGCCCGGAGTGTGTGGATAAAAAAGAGCTGAACTTGCTTATTAAGCGT | |
| | | TTTAAAAACATCTTGAATCAGATGATTAACGAAGATTCCCTTTGTCACGGCAAC | |
| | | GGTTCGATCATTACTACGATGAAGATGATCTATATGTACACCCAAGACACCGAG | |
| | | TGGAACTCTCTGATTAATCTGTGGTTATCAAATGTAAGTATCTATTCGACCTTA | |
| | | CAAGGCTATAGCATTCCAAAGCTGGGCGATGTAACAATTAAGGGGTTGTTTGAT | |
| | | GGCATTTGTGGTATTGGCTGGTTATACCTGTATTCGAACTTTAGCATTGAAAAC | |
| | | GTGCTGCTCCTCGAGGTCTAATAA | |
| pEG7079 | BsjM | 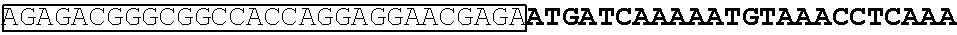 | 690 |
| | | GAGGCCATTAAAGGTTTGACCGTATCAGAACGTTATGACACTCTGAAAAATTCG | |
| | | GGAGTCAACCTGAATCTGAACATTTCGGCTTTGGAAGAGTGGCGCAACCGTAAG | |
| | | AATCTTTTAGCCGATGAGGACTTTACGGAGATGCTGACGGTGCTGGAATATGAC | |
| | | CCGGTGTATTTTAGCCACGCGATTAACGAGAACATCGAAGAACATATCGATATC | |
| | | TACAAGAGCAAAATTCTGGGGGAAAACTGGTTTATCGTGCTGAACGATATTCTG | |
| | | GACGAGCTCGATAATCCCATCGAATACAAGAAAGAGATGAATCACAGCTACCTC | |
| | | CTGCGTCCGTTCTTGCTCTACGCCGAAAAGGAGATGAACAAATACATTGTCAAT | |
| | | CGTAAGGAGTTACTTCCGGTGGAACCCCAGGTCATCCAACAGATCATGGAAAAT | |
| | | TTGGCCTCCAAACTGTTCGCCGTTTCTGTGAAAAGCTTTGTCCTGGAGCTGAAT | |
| | | ATTTCGAAATTGAAGGACGAACTGGCCGGCGAAACACCGGACGAACGCTTTCAC | |
| | | TCATTTATTCGTTTGATGGGTGAGAAAACGCGCCTGGTGGACTTTTACAACGAA | |
| | | TATATCGTTCTGAGTCGTATTCTGGTGAACATCACGATCTTATTCGTCAACAAC | |
| | | ATTATTGAGCTGTTTGAGCGCCTGCAGGAATCCAAGCTGGATATTGTTAAGAAA | |
| | | CTTGGCGTGCAGGAGGAGTTCAAAATCAGTAATATTAGCATTGGCGAAGGTGAT | |
| | | ACACATCAGCAAGGACGCTCGGTTATCGTTCTTACGTTCGTGAGTGGAAAGAAA | |
| | | GTGGTGTATAAACCAAAAAATCTGAAAGTTGTTTCTGCTTATAATTCTTTAATT | |
| | | GACTGGATCAACAATAAAAATAATATTCTGAAAATGCCTTCGTATAACACATTG | |
| | | ATTTATGATGATTTCGTGATCGAGGAGTTTGTCGAGAAACGTGACTGCAAAAGT | |
| | | ATCGAGGAGGTCAAAAAATATTATATTCGTTATGGGCAAATTTTGGGGATTATG | |
| | | TATATCTTAAATGGGAACGATTTTCATATGGAAAACCTGATTGCCTCGGGTGAA | |
| | | TATCCGATCATTGTTGACTTGGAAACGCTGCTTCAGAACATTATCAATTTTAAA | |
| | | AACAAACCATCAGCGGACTTGATCACCACCAAAAAGATGCTTAACCTGGTAAAC | |
| | | AGTACTCTGCTGCTCCCTGAAAAACTTCTGAAGGGCGACATCACGGACGAAGGA | |
| | | ATCGACATGTCAGCCTTGGCAGGGAAAGAACAACACTTGGAACGCCGCGAATAC | |
| | | CAGTTGAAAAACCTGTTCACCGACAACATGGTTTTTGATCTCGAAAAAGTGAAA | |
| | | ATCGAAGGTGCGAACAACATCCCGAAATTAAACGGTGAAAACGTTGACTACAGC | |
| | | ACCTATATTGATGAGATTGTGGTTGGGTTCGAAAATATCTGTAACCTGTTCATT | |
| | | CAATATCGCGACGAGTTACTGCATTCCGGCATCCTGGAGGAGTTTAAAGATGTG | |
| | | AAGGTTCGTCATGTGCTTCGCAATACGGTTGTTTATGCTAAGATGCTGGCGAAT | |
| | | ACATATCATCCAGATTACCTGCGTGATTCGTTGAATCGCGAACAGGTTCTTGAA | |
| | | AACATTTGGGTGCATCCGTTTGAGCGCAAAGAATTCATTAAGAGCGAGATGGAA | |
| | | GATATCCTCAACAACGACATCCCGATCTTTTTCTCATACGCGTCGTCTAAGGAT | |
| | | ATTATCGATTCGAATGGCAAACTGCACAAAAACGTTATGGAAATTTCGGGTTAC | |
| | | GAACGTTTTACCACCAAACTGAAGGAACTGAATCCCTTTCTGATTGAACAGCAG | |
| | | GTGAGCGTTATTAATATTAAAACCGGCCGCTATGGGGATAAGAAATTCGAAAAA | |
| | | AATTATAGCGTGCGCGACGTTGCAACGGAGAAAAAAGATAATCCGATTGATTTC | |
| | | CTGCAGGAGGCAATGAATATCGGCGATAAAATTTTGGAACATGCTATCATCTGT | |
| | | GATGAGACCAAAACGATTTCGTGGCTTACCATTAACAACCATCATGATAAAAAT | |
| | | TGGGAAATTGGGCCTATTTCCGGTGAATTTTATGATGGTCTGGCGGGAATTTCA | |
| | | CTCTTCTACCACTACCTCTATAAAAAATCCCACAATGTCGAGTATAAAAAAATT | |
| | | CGTGATTACGCGTTCAACATGGCGAAAGTCAAAGCCCTGTCACTGAAATACGAT | |
| | | AGTGGCTTGACCGGTTACGCTTCCTTGCTGTATACGGCACACAAGATTGTTCAG | |
| | | GATGAACCGCGGAAGCAATACAAAGACGTGATCAACGAAGTGTTCAAGTACATT | |
| | | GATGAGAGCAAAGTCGTGACCGCTAAGTATAACTGGTTGCATGGCACTGCCTCT | |
| | | ATTATTCATGTGTTATTGAACCTCTACGAGGACTCTCGTGATATGGCGTACCTG | |
| | | ACTAAATGTATTCAGTACGGCAAATATTTGGTCAAGCAAATCAAAGAACACAAG | |
| | | GATATGCTTGCGCCTGGCTTTAGCCAGGGCATCTCTTCGGTCATTATGGTTCTG | |
| | | GTGCGCTTAAGTAAAAAGTGTGAAGTCGAAGAATTTCTCGAATTAGCTCTGGAA | |
| | | TTAATGGAAATGGAACGCAACAAACTGGGAAACCTTTCTGAATCAAACTGGCTG | |
| | | AACGGCTTGGTGGGCATTGGCTTATCACGTATCAAACTGAAAGGACTGGATTCC | |
| | | AACTTACAGGTCGACAACGACATCGAACTCGTCCTGGATGGCGTCATGAACAGC | |
| | | TTGTACTCAAAAGATGATACTTTGAGCTGTGGTAACTCTGGCACAGTGGAATTG | |
| | | TTCCTGAGTCTGTTTGAACAGACGAAAAAGAAAGAGTATCTGGATATGGCGAAA | |
| | | GCAATCTGCGGGAAAATGATCGAAGAGAGTCGCATCTCCTTTGAGTATCAGACA | |
| | | AAGAGTCTGCCGGGTTTAGAACTGGTGGGCCTCTACTCTGGCTTAGCCGGAATT | |
| | | GGTTATCAATTCTTACGTATCTCGGACGTTGAGGATATTGCGAGCATTGCTACC | |
| | | TTAGATTAATAA | |
| pEG7127 | PsnB | 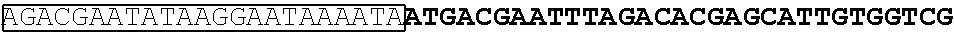 | 691 |
| | | TAGGAAGTCCGGATGATCTTCACGTCCAGTCAGTGACGGAGGGTCTGCGTGCAC | |
| | | GCGGTCACGAGCCTTACGTGTTTGACACCCAACGTTTTCCGGAAGAGATGACAG | |
| | | TGTCACTTGGTGAACAGGGTGCCTCTATTTTTGTCGATGGCCAGCAAATTGCAC | |
| | | GTCCGGCGGCGGTGTACCTCCGTTCACTGTACCAGAGCCCCGGCGCGTATGGGG | |
| | | TGGATGCCGACAAAGCGATGCAGGATAACTGGCGCCGCACATTGCTCGCTTTTC | |
| | | GCGAGCGTAGTACCCTGATGAGCGCTGTGCTTCTGCGTTGGGAAGAAGCGGGGA | |
| | | CTGCAGTGTATAATTCGCCACGCGCGTCGGCGAATATCACTAAACCGTTTCAGC | |
| | | TGGCGCTGCTGCGCGACGCTGGTCTGCCGGTACCACGTAGCTTGTGGACAAACG | |
| | | ACCCTGAAGCAGTGCGGCGGTTTCATGCGGAAGTGGGTGACTGTATTTACAAAC | |
| | | CGGTCGCCGGGGGAGCGCGTACACGCAAACTGGAAGCGAAAGATCTCGAAGCGG | |
| | | ACCGCATCGAACGCCTGAGTGCAGCGCCGGTGTGTTTTCAAGAACTGCTCACAG | |
| | | GAGATGATGTGCGTGTTTACGTGATAGATGACCAGGTAATATGCGCCCTGCGCA | |
| | | TCGTAACTGATGAGATCGATTTCCGCCAAGCAGAGGAACGTATCGAGGCCATCG | |
| | | AAATTTCAGATGAAGTAAAAGACCAATGTGTACGTGCCGCCAAACTTGTTGGCC | |
| | | TGCGCTACACCGGTATGGATATCAAAGCCGGCGCCGATGGTAACTATCGTGTTC | |
| | | TCGAACTGAACGCGAGTGCGATGTTTCGCGGTTTCGAAGGCCGTGCGAATGTGG | |
| | | ATATCTGTGGACCGCTGTGTGATGCATTGATCGCTCAGACCAAACGTTAATAA | |
| pEG7130 | AMdnC | 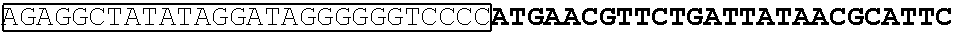 | 692 |
| | | CCACGATAACGAGAGCATTTCATTGGTAACCCAAGCCATTGAATCCCAGGGTGG | |
| | | TAAAGCATTTCGCTTCGATACCGATCGTTTTCCGACGGAAGTCCAGCTGGACAT | |
| | | CTATTACTCAAATACAGAGAAATGCGTGCTGGTGGCTGACGATCAAAAACTGGA | |
| | | TTTAAATGAAGTAACCGCGGTCTGGTATCGCCGCATTGCGATCGGTGGCAAAAT | |
| | | CCCGCCCACGATGGATAAGCAACTTCGTCAGGCCTCGATTCAGGAGAGTCGTGC | |
| | | TACAATTCAAGGCATGATAGCGAGCATTCGCGGCTTTCACCTTGACCCAGTGCC | |
| | | GAACATTCGTCGCGCTGAAAATAAGCAACTGCAGCTGCAGGTTGCCCGCAAAAT | |
| | | CGGACTGGATACCCCACGCACTCTCACCACTAATAATCCGCAGGCCGTGAAGGA | |
| | | ATTTGCGGCAGAATGCCAGCAGGACGTAATCACCAAAATGCTGAGTAGTTTTGC | |
| | | GATTTATGATGAGAAAGGCGGAGAACAGGTGGTTTTCACCAATCCCGTGAAATC | |
| | | TGAGGATCTGGAAAATTTAGAAGGTCTGCGCTTTTGCCCTATGACGTTTCAAGA | |
| | | GAAAATCGCAAAGGTTCTGGAGCTCCGGATCACCATCGTGGGTAAGTCAATTTT | |
| | | AACGGCTGCGGTGAATTCACAGGCCCTGGACAAATCCCGTTATGATTGGCGCAA | |
| | | GCAGGGCGTAGCATTACTGGATGCATGGCAGACCCATACGTTACCCCAGGACGT | |
| | | GGCTGATAAATTGCTTCAACTGATGGCCCATTTCGGGTTAAACTATGGAGCCAT | |
| | | TGACGTGATTCTGACCCCGGATAATCGCTATGTGTTCTTGGAGGTCAATCCGGT | |
| | | GGGCGAATTCTTTTGGCTTGAGCGTTGCCCAGGTCTGCCGATTAGTCAAGCTAT | |
| | | TGCTAAAGTGCTGCTTTCTCATATATAATAA | |
| pEG7132 | AtxBC | 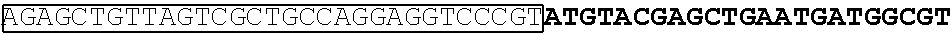 | 693 |
| | | AGGTTTGGCCCTCGTGGATCAGCATCCGATTTTTCTGGACCTGAAAACAGACCG | |
| | | TTACCTGTCGTTGAGTCCAGATGGGGCAGCAGTCCTGCTGGGAGCAGCGCCAGC | |
| | | CACCAAAGAGAGTCCACTGTTTCTCGGATTAGAATCCATTGGCTTGGTCAAAAA | |
| | | CGGTCCGTCAGGCCTTAAGCCTTGCCAAATTGCCGTAGCCACTGGGTCTGCACC | |
| | | GCCCCGTAAGGTGCAATTCGAGTCGTTGTCACTCCTGCTTTTGCGCTTAATTCG | |
| | | TGCACGTCTGGATCAACGTGCTCTTTTGAAGCGTGTGACCGACTTAAAGAAGGC | |
| | | CGGCACCATTGCCCAGACGAAGAACCGTGACTGCGCCTTGTCATTATTAGGTAG | |
| | | CGTGGAGACTGAGGCAAAGGCTTGTCGTACCCTTTTAAGTAGTACAGACAAATG | |
| | | CCTGCCCGACGCATTCGCAATTGCAACGCACCTGCGCCGTCGCGGAGTAGACGC | |
| | | CAAGTTAGTTTTCGGTGTGCGCCTGCCATTCGCGGCACATGCCTGGGTCCAGGT | |
| | | AGATGATATTGTAGTGGGTGATCGTCCCGACCGTATCCTTGCGTTCACCCCCAT | |
| | | 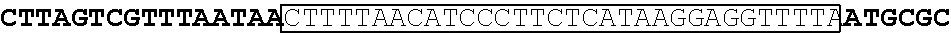 | |
| | | TATGTCGCGTCTTTCTTTGTTCGCGGACATGTCAGCACACCAGCACTGCGTCAC | |
| | | CCAGAGCCAAAGGGTTTCGCTTATGCAAAAGTCAGTGGCGGACTGAGCGTATGG | |
| | | AGCGATGCGCCGATTCGTCACCGTGCGCCCCTTATTACAGTGGGCGCGGTGTTC | |
| | | GATCGCGCGTCTTTTAAAGGGCTGGATTGCGACTTATCAGGTCTGCGTCAGGAT | |
| | | GGTCTTAATACATTGAAAGCGGAAACGTTCGGACCCTACCTGGCGTTAGAGGTT | |
| | | GCCGATAACGGCACCCTTCGCGTTTATCGCGATCCGTCAGGCGGCGCGCCTTGC | |
| | | TATTACCTGCAGACCGAGGACGGCTTCTGGCTTGCAAGCGATGCTGATTTGTTA | |
| | | TTCACTCATTCGGGCGTACATCCATCAGTAAGCTTACCGGGACTGATTGAACAC | |
| | | TTGCGTCGTCCAGAGTTCCAAAATGAGGGCACATGCTTAAACGTCAAGCAAGTA | |
| | | CGCCCTGGGGAGCAGGTTGATTTATCGCTCTCGGGCGAGGTCCGTGCCTGTTTG | |
| | | TTCCCGCCTGCATCATCCCTGCGCCCGCCTGAGTTGCACCGCGCATACGATGAC | |
| | | ATTAAGGCTGAGCTGCGCGCTCTGATTTTACGCAGCATTAAGGCCTATGCCAGT | |
| | | GATTTCCCTCACGTTGTTGTTAGCTTCAGCGGTGGTCTGGATAGCAGTGTTGTT | |
| | | GCGGCCGGCTTAGCGCAAACTTCCACTAAGGTCCTGCTTCACACCTTTAAGGGC | |
| | | CCAGATGCCAAAGGGGACGAGACTGCCTTCGCCGCAGAATGCGCGGCATATCTG | |
| | | GGTTTAAGCTTAGAGATTGATACTCTCAGTATCGATGACGTTGATCTGTCGGCA | |
| | | ACTATTTCCCCGCACCTGCCGCGCCCCAGCACATCATTCTTCTTGCCATCACTG | |
| | | CTGCGCGGTTTCTCTACCTCGAGCCAAACGCGCACAGGCGGGGCAATCTTTTCG | |
| | | GGAAACGGCGGTGACTCGGTCTTTTGTTTCATGCATAGCGCGACCCCGCTGGCC | |
| | | GATTTGATGTGTCGTCCGTCAGGTCTTACGCCGTTCATGCAAACATGGGCCGAC | |
| | | GTGCAAAAGCTTACCCGTGCCTCAGCGACCGAAGTGCTGCGTCGCGCGTTAAAG | |
| | | ACAGCCATGGCGCGTGGCTACATCTGGCCTGAATCCAATCTCCTCTTGTCCCGC | |
| | | GACACAAGCTCGAGCCGTTTAACACCTGACTCCGTTCTGTCGAGCCTTGAGGGG | |
| | | ATTCTGCCCGGTCGCTTGCGTCACCTCGCCCTGATTCGTCGTGCTCACAACACC | |
| | | TTCGAGCCATTCGCCCCTTGGCGTACGCCGCCAGTCGTTCACCCTCTCATGGCC | |
| | | AAGCCGATTCAAGCCTTCTGCCTTTCTCTTCCTTCATGGATGTGGGTCAGCGGT | |
| | | GGTAAAGACCGCTCGCTCGTGCGTGACGCGTTCGAAGGATTACTTCCAGATTCA | |
| | | GTGCGCCTTCGTAAATCAAAGGGAAGTCCTGCAGGCTTTCTGCATGCGCTGTAC | |
| | | CGCGCCAAGGGTCGTCAAATGATTGAGCGTATCCGTCACGGTTACCTGCGTCGT | |
| | | GAGGGGATCATCGATATCTCTACTGGCCCGGACGCATTGTTCTCGGAAGGGTTC | |
| | | CGCAATCCGCGTGTAATGCACCGTTTCTTTGAGCTCGCCGCAACTGAGGTGTGG | |
| | | ATCGATCACTGGCGCAACTGGCGCCGCCCCCGCACATAATAA | |
| pEG7133 | Cln1BC | 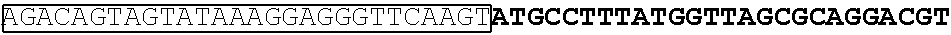 | 694 |
| | | CCACGCGGTCGCTCTGGACGAAGATATCGTGGTGCTGGATGCGGTGAGCGACGC | |
| | | ATACCTGTGTTTAGTTGGTGCCAGCGCTCTGATCAGCTTGGGCAGCGAGCGTTC | |
| | | CGTCAGTGCAGATCCGGTGGCCGCTGAGACACTTCGTGAGGCTGGTCTGGTGGG | |
| | | TCCACATCCTAGCGGCGCCACCCGACCAATACCTCCGAAGCCGACGATTGACTT | |
| | | ACCTGATGCAGCCCGTCAGGCGCAAGGTCGTGAATTACGTGCCGCCGCGTGGGC | |
| | | TGGCGCGGCAACCGCAATCGATTTCCGCCGGCGTTCATTTAGACAACTCCTCGC | |
| | | GAGAGCAGGGCAACGCCCGCCGGGTCAAGCAGCTGCTCCGGCTGATGAGGTATT | |
| | | GGCAGCAGCCGCAGTGTTCATGCGGTTACGTCCATGGTCACCCGTTGGAGGCGC | |
| | | GTGCCTTATGCGTTCGTATTACTTATTACGGCATTTGCGCATCCTCGGTTTCGA | |
| | | TGCCGATTGGATCATTGGTGTGCGTACGTGGCCATTTATGGCCCATTGCTGGCT | |
| | | GCAGGTCGGTGCCGTCGCACTCGACGATGACGTCGAGAGATTAACAGCATACAC | |
| | | 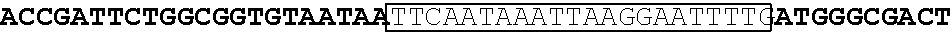 | |
| | | ACCTGGCTCTGTACTGGCCGCGCGGCATGCCCGGTGTAGCTGCAGACGCAATGC | |
| | | GGGCCGCCATCGAAGCTGAGGGCGCCTGGACCCTGGCGTTCGAGGCCTACCAGC | |
| | | TGGTAGTGTATGTCAAAGGGCCCCGAGCACCTAAAGTGCGTGCCCTGCCGGATC | |
| | | AGGGCGGGGTGGTCATTGGGGAACTGTTTGATACTGCAGCAACCCGCGAAGGAC | |
| | | GCGTGCAGGACTTTCCTATAGCGCTGATCAAAGACGTCGCAGCTCAGGATGCCG | |
| | | CACGTATTCTTGCTACCCATGCGTGGGGTCGTTATGTGGCTGTATTAAAAGCCG | |
| | | GTGATCGTCCGCCATGGATCTTTCGCGATCCAAGCGGGGCGGTGGAATGTCTGG | |
| | | CGTGGGTCCGCGATGAAGTGACCATCATTAGCAGCGATGTTGCAGCGCAACGAG | |
| | | CTTGGTCCCCTGATCGGCTGGCGATTGACTGGTCGGGACTGGGACGTGTACTGG | |
| | | CACGCGGAAACTTATGGGGAGAAATTTGCCCGCTGGCTGGCGTCACGGCGATTG | |
| | | CGCCAGGTACCGCACGGTGTGATCTCGGTGATGCAGCTCTGAGCCTGTGGCGCC | |
| | | CAGGAGATCATGCACGTCGTAGTCGTCATGATGTTTCCCCACGTGATTTGGCAA | |
| | | GAGTGGTGGATGCTAGCGTTGCAGCCCTGGCTAGAGATCGCAGCGCTATTCTGG | |
| | | TCGAAATCAGCGGGGGACTGGATTCCGCTATCGTTGCCACGTCGCTGGCTCGTT | |
| | | GTGGAGCCCCAGTTGTTGCTGGAATTAACCATTACTGGCCCGAACCGGAGGGTG | |
| | | ATGAACGTCGCTGGGCCCAGGACATCGCAGATCGGTGCGGTTTTCGCCTGATCG | |
| | | CGGGCCAACGTCAGCGGCTGTTGCTGGACGAGGCAAAGCTGCTGAGACATGCAC | |
| | | AGGGCCCGCGACCTGGTCTGAATGCGCAGGACCCGGACCTCGATCACGATCTGG | |
| | | CGGAACAGGCTAAAGCGTTGGGTGCCGATGCACTGTTCTCAGGGCAAGGTGGCG | |
| | | ATGGTGTGTTCTATCAAATGGCAAATGCTGCACTGGCAGCCGATATCCTCATGG | |
| | | GGAAACCTGCTCCTATGGGTAGAGCCGCGTCTTTAGCCGCTGTGGCTCGTCGGG | |
| | | CACGAGCCACGGTCTGGAGTTTGTGCGGCCAGGCTATGTTTCCGTCGCGCGCAT | |
| | | TTGCCGCTGGTATGCCGCCGCCAAGTTTCTTGAGCGCCGGTTTGGCGCCGCCAC | |
| | | CCGTGCACCCGTGGATTGCAGACCAGCGCGGTGTTTCACCGGCGAAACGTATTC | |
| | | AAATTCGGGGGCTGACCAATATTCAATGTGCTTTCGGCGATAGCTTACGGGGCC | |
| | | GAGCAGCAGATCTTTTATATCCGCTTATGGCCCAACCGGTCATGGAACTGTGTC | |
| | | TGTCTATCCCTGCACCGCTGTTGGCAGTAGGCGCATTGGATCGCCCTTTCGCAC | |
| | | GTGCGGCGTTCGCAGATCGATTACCTCCTCGTTCACTCGTTCGACGCTCAAAAG | |
| | | GTGATGTTACCGTGTTTTTCAGCAAAAGCCTTGCAGCAAGCCTGCCGGCCCTTC | |
| | | GTCCTTTCCTGCTGGACGGGCGCCTTGCAGAACAGGGTCTGATCGATCGAGCAA | |
| | | AACTGGAACCTCTGCTGCACCCCGAACCGATGATTTGGCGCGACTCAGTCGGCG | |
| | | AGGTAATGCTGGCAGCGTATCTTGAAGCCTGGGTGCGCGCATGGGAAGCCAAGT | |
| | | TGCGTGTTAGCTAATAA | |
| pEG7134 | Cln2BC | 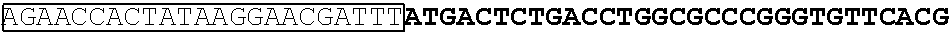 | 695 |
| | | CGGTAATGGTCGAAGATGATCTGGTTCTGCTGGATGAAGCAGCGGACGCTTATG | |
| | | TCTGTTTGTTGGATGGCGCCAAAGTGGTTAGCGTCCGGGCTGACGGTGCTCTGA | |
| | | GCTTCAATCCCCCACATGCAGCAGAAGATATGATCGCGGGTGGCCTCGTCGAAC | |
| | | CTTCATCAAGTGCCGCGGCGTCAGCAAACCCGCCGGCAAAACTCCCATGTACTC | |
| | | CGCTGGCGCGCTTATCGCGCCCGCGGCATGTAAAAGTGCGTCCGGCTGAAGCGG | |
| | | CCTTGTTCCTGATCCAAGCCTGGGGTGTTGCGCGTGCGGTACGTCGTTGGCCAA | |
| | | TGGCTAGATTATTAGAAGCATTACGTGGAGATCGTGCCGCAGAACCGGCGAAAG | |
| | | GCCGCCGATCGATGGCGGAGGCGTGCGCTGTTTTTGATGCGCTTCTGGCCTGGA | |
| | | GCCCTTTTGACGGTGAATGTTTGTTTCGCTCAGTATTACGACGTAGATTTTTAA | |
| | | TGGCACTGGGCCATTCGCCGGACTTGGTGATAGGCGTGCGTACCTGGCCGTTCC | |
| | | GCGCACATTGCTGGCTGCAGAGCGGAGTGGATGCCCTGGATGATTGGCCGGAAC | |
| | | GGCTCTGCGCATATCGCCCGATTCTGGCAGCTTCTGCAAGCCAGGGTAGATAAT | |
| | | 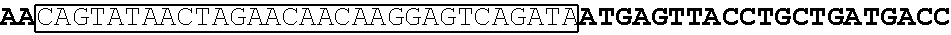 | |
| | | TGGCCGCCGGGGCAGCCGAGCGTAGAAGCTGATGCACTTCACGCAGCCTTTAAC | |
| | | GGGCAGGGTGGATGGAGCCTGGTTTTGGAACGATTCTGCCTGCGCGTATACGTG | |
| | | CGTGGCGCGGCAGCCCCTGCAGTTACCCTTACCCCGAAAGGAGGCGTGCTCATT | |
| | | GGTGAGATGTTTGATCGGGCTGCCACAGAAACGGGCGCCGTTGCCGCTTATGAT | |
| | | CTGAGCCGCCTGGGAGATGACGACGGTATGGCCGTAGCCCGGCGTGTGGTGGAC | |
| | | GAAGCGTGGGGGAGATATGTGTTGGTGCTGCCAGTTAAAGAACGCCGTCCAGTG | |
| | | GTTTTGCGAGAACCACTGGGCGCGCTGGATGCGCTGATCTGGCGCAAAGGCGAT | |
| | | GTCTGGTGCGTGGGGGCAGACGTACCCCCGGGTCTTGAACCAAAAGATCTGGGT | |
| | | GTGGAAGAGACTAGACTGACGCACCTGATCGCGGAACCGGATCTGGCATCTGCG | |
| | | AGCCTGCCCTTAACCGGCGTCGCGGCAGTGATGCCAGGTACTGCGGTCGATGAA | |
| | | ACCGGCCAGGTGCACCGTCTGTGGACCCCCGCGCGTTTTGCTCGCTCCCCTCGC | |
| | | ACTGACGCGTGGACTGCAGCCGAACGTATTCCGCTGGTTACCCGTGCGTGCATC | |
| | | GCGGCGCTGTCTGCGAATCGAAGTGGTATTCTGTGCGAGATTTCGGGCGGCCTG | |
| | | GATAGCGCTATTGTTGCGACCTCTCTGAAAGCGGAAGGTGCGAAGATTAGTAGC | |
| | | GGGATCAACTTCCATTGGCCCCAGGCTGAAGCAGATGAGCGCCCGTACGCACGC | |
| | | GCTGTTGCGAAAAGCGTGCGAACCCGGTTACAGGTGGTAGCGAGTCGTGTAGCG | |
| | | CCCGTTGACCCGGAAACGTTTGATGAGATCGTGGTCGCGCGACCAAGTTTTAAT | |
| | | GCCATTGATCCAGTCTATGATACCGTACTGGCCCAACGTCTGATTCAGGGCGGT | |
| | | GAAGGAGCCCTGTTTACCGGACAAGGTGGTGACGCAGTTTTCTATCAGATGCCA | |
| | | GCACCACAACTTTCGTTGGATTTGTTGGCTCGTGGCCCCCGCCGCCGCGGTCTT | |
| | | ATGGGATTATCACGCCGCACCAACCGCAGTGTCTGGTCGTTGCTGCGCATGGGC | |
| | | TTACGTGCACCCGTACGAGCAACCTTTCCCTACGGTGCGAGAGGTGCCGATCGT | |
| | | CCTCCGATGCACCCGTGGCTGGAGGACGCGCGTGGTGTTGGGGCCGCGAAACGG | |
| | | ATTCAGATCGAAGCGCTGGTTGCTAACCAGGCCGTGTTTGAAGCATCTCGTCGC | |
| | | GGTGCGGCGGCTCATTTGGTGCACCCACTGCTGTCGCAACCGCTTGTGGAGCTG | |
| | | TGCCTTTCAACCCCAGCGGCCGTGCTGGCGGGTGCCGAACAAGATAGAGCATTC | |
| | | GTGCGTAGCGCTTTTCGTGCGCAACTGCCACGCCTGGTCTTAGATCGTCAAAGC | |
| | | AAAGGAGATCTGAGCGTTTTCTTTGCTAAAGGTGTGGCGCGGAGCTTGCCGGGC | |
| | | TTGCGTCCGCGTCTGCTCGAAGGACGCTTAGCGGCACGTGGCCTGATCGACGTG | |
| | | GAAGCGTTATCACAAGCGATGCAGCCAGAAGCGATGATTTGGCGTGACGGTTCG | |
| | | GCCGAAATCCTGTGCCTTGCTGTTCTGGAATCATGGCTCCGCTCTTGGGAGGCT | |
| | | CGTGGTGCATAATAA | |
| pEG7135 | Cln3BC | 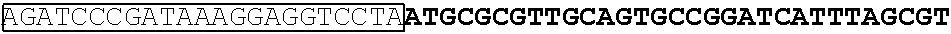 | 696 |
| | | ATTGCGTAAAACAAGGTGGAGTTACGTTTCTGGACGTCCGCGGGGATCGTTACT | |
| | | TCGGCCTGCCGCCGGTGCTGGAACACGCGTTCGTTGCCATTGCCGAGGCGGATT | |
| | | TTCTGCTGAAAGAACCAAATTCACTTCTGGAGCCACTCGAAGCACTGGGTGTCT | |
| | | TAGTGCGAGGCCAAGCCCGCCGTGCCGATCTGACAATTCCGTCTGCAAATCTGT | |
| | | CATGGGTGGATGAGGTCAGCCCGACCCCACCACGTCTTGACCCTGCGTCACTCG | |
| | | TCGCAACCGTCACGTCTGTTATTCGAACGCGTCTGAGCCAAAAGAGTAAGTCCT | |
| | | TGCAGGCTCTCTTGGAAGAGGTCCGTACCCGCCGTCCGGGATCGCCGGCCCATA | |
| | | ATTGGCAGCTGATGCGTCGTCTGACGGCTGGATTCCGTGCATCGCGTGCTTGGG | |
| | | CGCCGATAGAACCCATCTGCCTCCTGGACAGCTTGGCGTTACTGGATTTTCTGC | |
| | | ATCGCCGTGGCCTGTATCCGCATATTGTTTTCGGTGTGATCCGCCAACCGTTTG | |
| | | CCGCTCATTGTTGGGTGCAAGCTGATGATGTAGTCCTGAATGACCGGCTGGATC | |
| | | 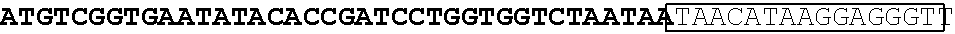 | |
| | | 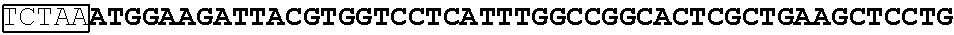 | |
| | | CACGCGACTTGATTCGTCGCCTGCCGAAACTCAAAACCGTCATTGAAACTAGCG | |
| | | GATTGGTGGTACTGCGCCCCGAAAATGGTGCGGGTCTGCGGGTAGGCGGGAACG | |
| | | GTGTGGTCCTGGGTAGCGTCTTTCGCACCGGCGGTGATCGCGAAACTGTTGCGG | |
| | | AATTTTCGGAATCGGAAGCATCCGCGATCGCCACGAGTCGTGGTCAGCAGTTAG | |
| | | TGACAGAGTTCTGGGGTGGCTACCTGGCTGTTCTTGGAGATGCTTCGCGTTCCG | |
| | | AAGTGATGGTCCTGCGAGATCCTTCAGGTGCAATGCCGGCTTATTGTTTAGTTC | |
| | | ATGGCGAAGTCCAGATCATCTGCTCTCGCTTGGAGGTCCTGGAGGACGCAGGAC | |
| | | TGGGGCAGCAGGCGCTGAACTGGGACGTGGTGGCGCAATTACTGGCCTTCCCAA | |
| | | ACCTTCGAGGTCGCTCAACGGGTCTTAAAGGCGTGGAAGAATTACTTCCCGGTT | |
| | | GCCGTCTGACATTTACGGGAGGACTGAAAACCGAAACGCTGACCTGGAACCCGT | |
| | | GGCTTTTTGCCCGCCCATCTGCGCAAGCGCCTGAACGTGGAGTTGCGGCGACCG | |
| | | CCGTGCGTCAGGCGGTGGAAGTAAGCGTTCGAAAATGGGCTGATCAGAGTTCAC | |
| | | CGGTACTTTTGGAATTGTCAGGCGGGCTGGATAGTAGTATCATCGCCTGCTGTC | |
| | | TGGACGAACCGCGCACCGCGGCCACCTTCGTGAACTTTGTCACACCGACGGCCG | |
| | | AAGGCGATGAACGAGGATATGCACGTCTGGTTGCCAAGGCAGCAGATAAACAAC | |
| | | TGATCGAGCAGGACATCCGGGCTGACGAAGTAGATGTTACCCGTCCAAGACCTG | |
| | | GCCGCCATCCTCGTCCGGCCAGTCAGGCGCTGTTACAGCCGCTGGAACAGGCTT | |
| | | GCGCTGAACTGGCACCTCAGTTGGGTGCGAGAAGTTTCTTCTCCGGTCTGGGAG | |
| | | GAGACAACGTGTTTTGTAGCATTGCAACCGCAAGCCCGGCTGCGGATGCACTTT | |
| | | TGACTAGCGGTCTGGGCCGACAGTTCTGGGCCGCAATCGGGGACCTGTGTGCAC | |
| | | GTCATAACTGCACCGTATGGGCAGCCTTAAGCGCCACGCTGAAGAAACTGCTCC | |
| | | GCTCAGATCGTCGTCTGGTGATCAAACCAAACCTGGATTTTCTGTCCTTTCGGG | |
| | | AGGACGCCATAGACCGTCCGGATCACCCATGGCTTGAAGTGGCCGCCGATCGTC | |
| | | TGCCGGGGAAACGCGAACATGTCGCAAGCATTCTGTTGGCGCAAGGCTTCCTGG | |
| | | ATCGTTATGAGCACGCTCAGGTTGCTGCCGTCCGCTTTCCCTTGTTAACGCAAC | |
| | | CGGTTATGGAGGCTTGTCTGCGCGTGCCGACCTGGATGGCAAACCACCAGGGTC | |
| | | GCAATCGGGCGGTCGCACGCGATGCCTTCTTTGATCGCTTGCCCCCGAGAGTAC | |
| | | GTGATCGGCAGACAAAAGGAGGTTTGAACGCGTTTATGGGTGTTGCGTTCGAAC | |
| | | GCAACCGTCAGGCCTTAGCTCGTCATCTGTTAGACGGGCGCCTGGTACAGCGTG | |
| | | GCCTGATAGATGCAGTGGCAATAAAATCGGCGCTGGCCTCACCAGTCCTGGAAG | |
| | | GAGGAGCCATGAACCGCTTACTGTACCTGGCCGATGTCGAATCCTGGGTACGCT | |
| | | CATGGGAAGATGTGTAATAA | |
| pEG7136 | CsegBC | 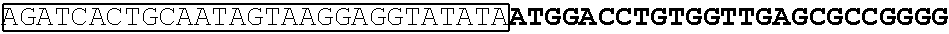 | 697 |
| | | TCTATGCTGTCATGATCGATGATGATGTAGTTTTCCTGGACGTCGCCACCAATG | |
| | | CATACTTCTGCCTCCCAGCCGTTGGGAGCGTGTTGGCACTCGAAGGTCGTTCGC | |
| | | TGCGTGTGGCGGCTCGCGAACTGGCAGAAGATCTTATTCAGGCAGGCTTAGCAT | |
| | | CCGCGGCTGCGGCAATCGAACCCCCACCGAGCACACCAGCCCCAGTTCGCACTG | |
| | | CGCGTGCGGTATTGGAAGCTCTGCCGGCGCGTGAAAGACCACGTCCACGTCTTG | |
| | | CCCACTGGCGTCAGGCGATTATGGCTGGCTTGGCGTCCCGTGCCGCTGAACGTC | |
| | | GACCATTCGCGCAGAGACTGCCGCCGCCTTCAACGGGGGTTTCACCTCCGGCAT | |
| | | CAGAAGGCCTGCTTGCCGATCTGGATGCGTTCCGTCGACTTCAGCCATGGTTGC | |
| | | CGTTCGACGGTGCTTGTCTGTTCCGTAGCCAAATGCTGCGCGATTATCTCCTTG | |
| | | CGCTGGGTCACCGCGTTGACTGGATTTTCGGTGTACGTACGTGGCCGTTTGGTG | |
| | | CCCACTGTTGGTTGCAGGCCGGCGACCTGGTGCTGGATGATGAGGCCGAACGTC | |
| | | 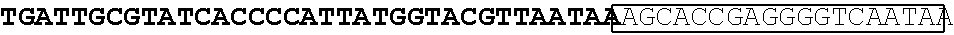 | |
| | | 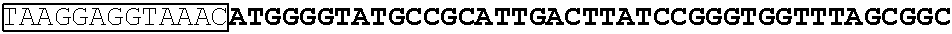 | |
| | | AGCAGCGTTTGATGAGATGGTAGAAGCACTGATCGATGCTGGATGGACCTTGGC | |
| | | GTTGCGTGCGTTCAGACTCGCCGTTCTCACCGATGGTCAGGCTCCAGCCGTGTC | |
| | | GCCGCTGATGGGCAGAGGCGGCGTAGCAGGCGTTCTCATCGGCGAAGCGTTTGA | |
| | | TCGTCGCGCCACATTAGGTGGCGCGGTCGCACGTGCCGCGCTGGATGGTTTGGC | |
| | | TGACATCGATCCGCTGGAAGCAGGTCGCCATCTGATTGAAACCGCGTGGGGCGG | |
| | | CTACGTGGGTATGTGGATTGGTCGGGCCGAAGCTGGTCCGACACTGCTGCGCGA | |
| | | TCCTAGTGGCGCGCTCGAAGCCTTAGCGTGGCGCCGTGACGGTGTAACCGTTAT | |
| | | GTCAGCGCGCCCGTTGACGGGGCGCGCAGGCCCAGCTGATTTAGCAATCGATTG | |
| | | GCCACGTATCGTGCAGATTCTGGCCGATCCCATTTCCGCGGCTCTCGGCCCGCC | |
| | | CCCTCTGACTGGCTTAGCGACCATAGACCCGGGCGCGGCGGTTCATGGCGCGGA | |
| | | TGGCCAAGAACGCTCAGTGCTGTGGACCCCAGCTGCAGTTGTCCGTGGTGCTCG | |
| | | TCACCGTCCTTGGCCAAGCCGTCAGGATCTGCGTCGCACCATCGATGCGACTGT | |
| | | CGCGGCACTGGCCTCGGATGCGGGCCCGATTGTCTGCGAAATTTCAGGAGGTCT | |
| | | GGACTCGGCCATAGTTGCGACTAGCCTTGCGGCGTCCGGTCTGGGTCCGCAGCT | |
| | | GACAGTGAATTTTTACGGTGACCAGCCTGAAGCTGATGAACGCGGATACGCTCA | |
| | | AGCCGTCGCCGAACGTATCGGTGCGCCTCTGCGGACCCTTCGTCGAGAGCCGTT | |
| | | CGCGTTCGATGAAACCGTGCTGGCAGCCGCTGGACAGGCCGCACGTCCGAATTT | |
| | | TAACGCCCTCGATCCTGGATACGATGCCGGGCTCGTGGGTGCCCTGGAAGCTAT | |
| | | CGATGCTCGTGCATTATTTACGGGCCATGGCGGTGATACCGTGTTTTATCAAGT | |
| | | GGCGGCCAGTGCCTTGGCCGCAGACTTACTGGGCGGCGCACCATGTGAAGGTAG | |
| | | CCGCCGTGCACGTTTAGAGGAAGTAGCTCGGCGGACCCGACGCTCGATTTGGAG | |
| | | TCTTGCATGGGAAGCGTTTTCTGGTCGACCCAGCACTGTAAGCATTGAAGGTCA | |
| | | GTTGCTTCGACAGGAAGCAGAGAGAATTCGGCGCGTCGGCCTGACCCATCCGTG | |
| | | GGTTGGAGGCCTGTCGTCTGTGACCCCTGCGAAACGCCAGCAAATCCGCGCGCT | |
| | | GGTCAGTAACCTGAACGCGCATGGCGCCACTGGTCGCGCCGAACGCGCTAGAAT | |
| | | CGTGCACCCGCTTTTAGCTCAGCCGGTGGTTGAAGCCTGCCTGGCGATTCCTGC | |
| | | CCCTATCCTCAGTGCGGGCGAAGGAGAACGCTCATTTGCGAGAGAAGCCTTTGC | |
| | | AGACCGTTTGCCACCGAGCATTGTGGGCCGCCGAAGCAAAGGGGAAATTAGTGT | |
| | | GTTTCTTAACAGATCTTTAGCAGCCAGCGCCCCCTTTCTGCGTGGCTTTTTACT | |
| | | TGAAGGACGGCTGGCGGCTCGCGGGCTGATTGATCGTGACGAACTTGCAGCCGC | |
| | | GCTGGAACCGGAAGCAATCGTCTGGAAGGATGCGTCACGCGACCTGCTTACTGC | |
| | | GGCGGCCCTGGAGGCGTGGGTCAGACATTGGGAAGCACGTATTGGCGAGGGGGA | |
| | | AGCAGCGGAAGGTGAGCGTGCTGCCGGTCGTGGTACCGCAGCGACGGGACCGCG | |
| | | TACAAGCGCGCGGAAGGCGAACACCCGTTAATAA | |
| pEG7137 | PadeK | 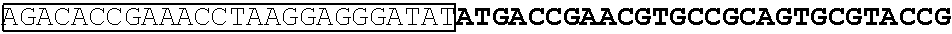 | 698 |
| | | ACCATTATAAAGCCTTTGGGTTTAGAATTGAAAGCGATTTCGTGCTCCCGGAAC | |
| | | TTCCGCCCGCAGGCGAACGCGAACCGCTCGATAATATTACGGTTCGTCGTACCG | |
| | | ACCTGCAGCCGCTCTGGAATTCTAGTATCCATTTTTACGGAAACTTTGCCATTC | |
| | | TGGATCACGGACGCACGGTTATGTTTCGAGTTCCGGGTGCTGCTATCTATGCGG | |
| | | TACAGGATGCTAGCAGCATATTAGTGTCCCCATTCGATCAGGCAGAAGAAAACT | |
| | | GGGTACGTCTTTTTATTCTGGGTACCTGTATTGGGATCATCCTGCTGCAGCGTA | |
| | | AGATTATGCCGCTGCACGGTAGCGCCGTTGCCATTGATGGCAAAGCCTACGCGA | |
| | | TTATCGGCGAATCTGGTGCCGGCAAAAGCACTCTTGCACTGCATCTTGTCAGTA | |
| | | AGGGTTATCCATTGCTTTCGGATGATGTGATTCCGGTCGTTATGACCCAGGGCT | |
| | | CCCCCTGGGTGGTGCCGTCGTACCCGCAACAAAAACTTTGGGTGGACACTCTGA | |
| | | AGCACATGGGAATGGATAATGCAAACTATACGCCGCTGTACGAACGTAAAACGA | |
| | | AGTTCGCGGTGCCCGTGGGCAGTAATTTCCACGAAGAACCGCTGCCGTTAGCTA | |
| | | GCATTTTCGAGCTTGTCCCGTGGGATGCGGCAACGCACATTGCCCCGATCCAAG | |
| | | GGATGGAACGCTTTCGTGTCCTGTTCCACCACACTTATCGGAACTTTCTGGTTC | |
| | | AGCCGCTGGGTCTTATGGAATGGCATTTTAAAACTCTGAGCTCGTTCGTTCACC | |
| | | AAATTGGAATGTATCGTCTGCATAGACCTATGGTCGGATTCAGTACCTTAGATT | |
| | | TAACGTCGCACATTCTGAATATAACGCGTCAGGGAGAGAACGATCAATAATAA | |
| pEG7138 | ThcoK | 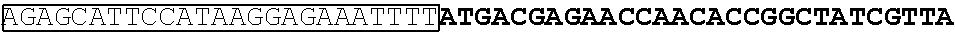 | 699 |
| | | TCGCGCGTTCGGCCTGCGCATAGACTCAGATATTCCGCTGCCAGAATTAGGGGA | |
| | | CGGTACGCGCCCTGATGGTGACGCGGATCTGACGGTCGTCCGGTGTGGGGAAGC | |
| | | GGAGCCGGAATGGGCTGAAGGTGGTGGCGGGGGTCGTCTGTATGCCGCTGAAGG | |
| | | CATTGTATCTTTTCGCGTGCCGCAGACGGCAGCGTTCCGTATTACTAATGGAAA | |
| | | TCGCATCGAGGTGCATGCCTACTCGGGGGCTGATGAGGATCGAATACGCCTGTA | |
| | | CGTGTTAGGGACCTGTATGGGAGCGCTGTTACTGCAACGTAGAATCTTACCGCT | |
| | | TCATGGTTCGGTCGTCGCCCGTGATGGTCGTGCGTATGCCATAGTTGGCGAAAG | |
| | | CGGAGCGGGCAAATCCACGATGAGTGCAGCACTTCTCGAACGTGGATTCCGCCT | |
| | | CGTTACGGATGACGTGGCCGCCATCGTGTTCGATGAGCGTGGGACCCCACTGGT | |
| | | TATGCCGGCTTATCCACAGCAAAAACTGTGGCAGGATTCCCTGGACCGTCTGCA | |
| | | AATTGCGGGCTCGGGCCTTCGTCCGCTGTTCGAACGCGAAACGAAATACGCTGT | |
| | | ACCCGCGGATGGGGCATTCTGGCCCGAACCGGTTCCATTGGTGCACATTTACGA | |
| | | ACTGGTTCATAGCGATGGTCAAACGCCTGAACTGCAGCCGATTGCCAAATTAGA | |
| | | GCGTTGCTATACCTTGTATCGCCACACATTTCGTAGAAGCCTGATCGTCCCCAG | |
| | | CGGCTTAAGCGCCTGGCATTTTGAAACGGCAGTGAAACTTGCGGAGAAAACGGG | |
| | | GATGTACCGTCTTATGCGCCCGGCCAAAGTTTTCGCGGCTCGCGAATCTGCTCG | |
| | | GCTGATTGAAACTCACGCCGATGGTGAAGTGTCACGTTAATAA | |
| pEG7139 | StspM | 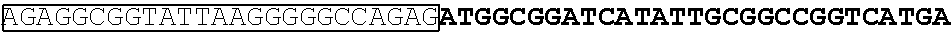 | 700 |
| | | CACCGTCCTGAGCCTGGCCGAACGGACAGGTACCGATCCAGATCTGCTGGGCCG | |
| | | TGTGTTGCGCTTCCTCGCTTGTCGTGGTGTTTTCGCCGAGCCTCGCCCAGGTAC | |
| | | TTATGCCTTGACCCCTCTGAGCTTAACTTTACTGGAAGGCCATCCGTCCGGTTT | |
| | | AAGAGAATGGTTGGATGCGTCGGGTGCGGGAGCGCGCATGGACGCGGCAGTTGG | |
| | | AGATCTGCTTGGCGCCCTCCGCTCGGGTGAACCGAGCTATCCACGTCTGCATGG | |
| | | TCGTCCGTTTTATGAAGATCTGGCGCTGCACAGCCGAGGCCCTGCTTTTGATGG | |
| | | ACTGCGTCATACGCACGCCGAATCGTATGTTGCCGACCTGCTGGCAGCCTACCC | |
| | | GTGGGAACGCGTTCGTCGCGTGGTTGATGTAGGCGGTGGGACCGGCGTATTGGT | |
| | | CGAGGCGCTTATGAGAACTCATGCGACCCTCCGTACAGTACTGGTCGATCTTCC | |
| | | AGGCGCGGTGGCTACCGCTACCGCTCGAATTGCGGCTGCGGGTTTTGGCAATAG | |
| | | ATATACACCGGTCACGGGTTCCTTCTTTGATCCGCTGCCTGCGGGGGCGGATGT | |
| | | TTACACCCTGGTTAACGTGGTTCACAACTGGAACGATGAGCGTGCCTCAGCTCT | |
| | | GCTGCGTCGGTGTGCGGATGCGGGTCGCCGCGACAGTACGTTTGTTATCGTGGA | |
| | | ACGCTTAGCGGACGATGCAGACCCTCGTGCCATCACCGCCATGGACCTCCGTAT | |
| | | GTTCCTTTTTCTGGGCGGTAAAGAGCGCACGGCCGCACAGATTCGCGAAGTAGC | |
| | | TAGTGCGGCTGGCATGGCCCACCAAAGCACCATTAAAACACCGTCTGGCCTCCA | |
| | | CTTACTTGTTTTCCGTAAGAAACGTTTCGCTGCTCGCGGTCACGGTCGTCGCAT | |
| | | GGTGACCTAATAA | |
| pEG7141 | LenG | 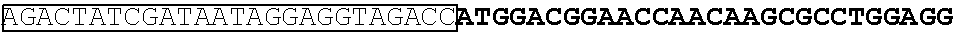 | 701 |
| | | ACAAGTGGTTTGATATTAACTTCCTGGAAATGTATACACGCAGCTGCCTGAAAA | |
| | | CTTTTGGCTACTTCGACGAAATTCTGATCGTGAAGAAACGCATCGAGGTCCTGA | |
| | | AGAACGTGCTTGAAAAACAGTACTTGTCTACCAATGATTATGCTGAGGAGTTTT | |
| | | TCGAGCTGAATACCACCTTGGAGAGCATAAAAGAATACATCAAACTGAATCTGG | |
| | | TCATCGAGAAAGAACCGATCTCAATTTGCATTATGGTCAAAAACGAAGAACGTT | |
| | | GCATCAAGCGCTGCATTGATAGCGTTGAAATCCTCGCCGAGGAGATAATCATTA | |
| | | TCGATACCGGCTCTACGGATAATACCATTAACATTATTGAGGAATGCGCAAACG | |
| | | ACAAAATTAAAGTGTTCTCAAAAGAATGGCGTAACGATTTTTCCGAAATTCGGA | |
| | | ACTATGCCATCGAGAAAGCGAGTAGCGAATGGCTGGTGTTTATAGATGCCGATG | |
| | | AATATCTGGACGAAGCCTCGGTGCTCAACCTGCTCAGTACGCTCAACATCTTTA | |
| | | ACAATCATAAGCTCAAAGACTCTATTGTCCTGTGCCCCATGATCAACGAAGCCA | |
| | | ATAACACCATCCATTTCCGTACCGGGAAATTTTTCAGAAAAGACTCCGGGATTA | |
| | | AATTCTTTGGTACCTGCCATGAGGAGCCCCGCATTAAAGGCATGCCGAATTCTA | |
| | | CCCTGCTGATTCCGATCAAGGTTGATTATCTGCATGACGGCTACCTGGCAAAAG | |
| | | TACAATCAAATAAAGACAAGAAAACCCGTAACATCGAACTGTTAGAAGGTATGG | |
| | | TGGAACTGGAACCGGATAATCCTCGTTGGGCGTATATGTTTGTGCGCGACGGAT | |
| | | TTGCAATCCTCGATAACGAATACATTGAGAAAACTTGTTTGCGGTTTTTACTGC | |
| | | TGGACAAAAACGTACGCATCTGCGTCAACAACCTGCAAGACCATAAATTCACTT | |
| | | TGTCACTCCTGACGATCCTGGGCCGCCTCTATCTGCGCGAGTGCGAATTCGAGA | |
| | | AAAGCAATCTGATAATTCGCATTCTTGACGAACTCATCCCTAATAGTCTGGATG | |
| | | GTAAATTTCTGGCATTCATGGAGCGATTCAGCAAACTGAAAATTGAGATTAATA | |
| | | CGCTGTTAACGGAGGTCATCGAATATCGTCGTAACCACGAAGTAGATGAAACCA | |
| | | GTTTAATCAACACACAAGGCTACCATATCGACTATGTTCTGTCGATTTTGCTGT | |
| | | TCGAAACGGGTAATTACGCGCAAAGTAAGAAATACTTCGATTTCCTGCAGGAGA | |
| | | ACCATTTTCTGGAAGAACTGTTTCAAGACAGCTCTTATTCTATCATACTGAAAA | |
| | | TGCTCGAGTCAGTAGAAGATTAATAA | |
| pEG7142 | PalS | 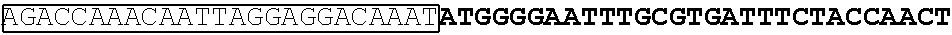 | 702 |
| | | GATGAAAGATAACTATGCGGACTCTAATCTGTTCAAGGATTTGAATCTGATCCA | |
| | | CAATATCTCCAACGACATCCAAATTGGAATTAATTGCGATTTCTCTGAAATGCT | |
| | | GGGAGAACTGGTAGGTAATTACGATTCCCTGAACTATCCGTCAATCACCTGTGG | |
| | | TATTCTGACGTATAATGAAGAACGCTGCATTAAACGTTGTCTGGAAAGTGTGGT | |
| | | GAACGAATTCGATGAGATTATTGTCTTGGATAGTGTATCCGAGGACAATACCGT | |
| | | GAAAATTATCAAGGAGAATTTCAACGATGTCAAAGTCTACGTCGAGCCATGGAA | |
| | | GAACGATTTTTCATTTCACCGCAACAAGATCATTAATCTCGCAACGTGCGACTG | |
| | | GATCTACTTTATCGACGCGGATAATTATTATGATTCGAAGAACAAGGGTAAAGC | |
| | | CATGCGCATCGCTAAGGTTATGGATTTCTTGAAAATCGAAGGCGTTGTGAGCCC | |
| | | AACGGTCATTGAGCATGACAATAGCATGAGCCGTGATACCCGTAAGATGTTTCG | |
| | | TCTGAAAGATAACATTCTGTTTAGCGGTAAAGTTCATGAAGAACCGGTGTATGC | |
| | | CAATGGTGAGATCCCCCGGAACATCATAGTAGACATCAACGTGTTTCACGACGG | |
| | | CTATAACCCAAAGATTATCAACATGATGGAAAAGAACGAGCGCAATATCACCCT | |
| | | GACTAAAGAGATGATGAAGATCGAACCGAACAATCCGAAATGGCTGTACTTCTA | |
| | | TAGCCGCGAACTCTATCAGACGCAACGTGACATTGCCCTTGTGCAAAGTGTACT | |
| | | GTTCAAGGCACTGGAACTGTATGAAAACAGTTCATATACGCGTTATTATGTTGA | |
| | | CACCATTGCCTTACTGTGCCGAGTGCTGTTCGAATCTAAAAACTACCAGAAACT | |
| | | TACGGAATGTCTGAACATCCTGGAGAACAATACGCTTAACTGTTCCGATATCGA | |
| | | TTACTATAATTCAGCGCTGCTGTTCTACAACCTGTTACTGCGCATCAAGAAAAT | |
| | | TAGCTCCACCCTGAAGGAGAACATTGATATGTACGAACGTGACTATCATAGCTT | |
| | | TATCAACCCCTCGCATGATCACATTAAGATTCTGATATTAAATATGCTCCTGCT | |
| | | GCTCGGCGATTACCAGGATGCCTTTAAGGTTTACAAGGAGATCAAGTCCATTGA | |
| | | GATTAAAGATGAGTTTCTGGTGAACGTGAACAAATTCAAAGACAATCTTCTGAG | |
| | | CTTCATTGACTCCATTAACAAAATTTAATAA | |
| pEG7143 | SgbL | 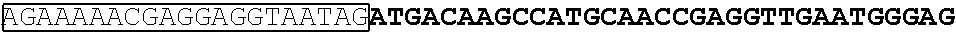 | 703 |
| | | GACCTTCTGCGCCAAGCATTACACGCAACTGGTACAGGTGCTCGTTGGGCTGTA | |
| | | GAGGCGGACGAGATGTGGTGCCGTGTCGCCCCGGTGCCTGGAACTCGCCGCGAG | |
| | | CAAGGATGGAAGCTTCATGTAAGCGCGACGACCGCGAGTGCGCCCGAAGTCTTA | |
| | | ACTCGTGCATTAGGCGTACTTCTGCGTGAAAAGTCCGGGTTCAAATTTGCCCGC | |
| | | TCACTTGAACAAGTCTCGGCCTTGAATAGTCGTGCTACGCCCCGTGGTAGTTCG | |
| | | GGTAAATTTATCACAGTATACCCCCGCTCAGACGCCGAAGCCGTCGCACTGGCT | |
| | | CGCGACCTGCATGCGGCAACGGCCGGCTTGGCTGGGCCCCGTATTCTTTCCGAT | |
| | | CAACCATACGCCGCGCACAGCCTGGTGCATTATCGTTATGGGGCTTTCGTGGGA | |
| | | CGTCGTCGCCTTTCAGATGACGGGCTTTTAGTTTGGTTTATTGAGGACCCAGAT | |
| | | GGCAATCCCGTGGAGGATAAACGCACCGGACGTTATGCGCCGCCTCCCTGGGCT | |
| | | GTATGTCCGTTTCCTGCGAGCGTCCCCGTTGCGCCCCATGACGGCGAAGCTACG | |
| | | AGTCGTCCTGTTGTCTTAGGTGGTCGCTTCGCGGTTCGTGAAGCCATCCGTCAA | |
| | | ACGAATAAAGGGGGCGTCTATCGCGGGTCGGACACACGCACTGGCACCGGCGTG | |
| | | GTTATCAAAGAGGCGCGCCCACATGTTGAAGGAGACGCCAGTGGGGGCGATGTT | |
| | | CGTGACTGGCTTCGCGCAGAGGCGCGTACGCTTGAAAAATTAAAAGGTACCGGC | |
| | | TTGGCACCAGAAGCGGTGGCGTTGTTTGAGCACGCTGGCCACTTGTTCTTAGCC | |
| | | CAAGACGAGGTCCCGGGGGTTACGTTACGCACCTGGGTAGCGGAACACTTCCGT | |
| | | GACGTTGGAGGAGAGCGCTATCGTGCCGACGCCCTGGCTCAGGTGGCTCGTTTA | |
| | | GTTGATTTAGTCGCGGCTGCTCATGCACGTGGCTTGGTCCTGCGCGATTTTACA | |
| | | CCAGGGAACGTGATGGTCCGTCCAGACGGCGAATTGCGCCTTATTGATTTAGAG | |
| | | CTGGCGGTTCTTGAGGATGAGGCCGCATTGCCTACCCACGTCGGTACCCCGGGG | |
| | | TTTTCGGCACCCGAACGCCTTGCAGACGCTCCAGTGCGTCCTACTGCTGACTAC | |
| | | TATTCTCTGGGAGCCACAGCTTGTTTTGTCTTGGCCGGTAAAGTCCCTAATTTA | |
| | | CTTCCTGAAGAACCCGTGGGTCGCCCATCGGAGGAGCGTCTTGCTGCCTGGTTG | |
| | | ACTGCATGTACACGTCCGCTGCGCCTGCCAGATGGAGTCGTTGACATGATCTTG | |
| | | GGGTTAATGCGCGATGATCCTGCAGAGCGCTGGGACCCATCCCGCGCGCGTGAA | |
| | | GCACTGCGCAAAGCTGACCCGACAGCACGCCCCGGGGATGCTGATCGCACTGCA | |
| | | GTACGTCGTACGGGTTCGTCGGCAGTGGCCGGGCCAGTTCCTGACTCACGTACA | |
| | | GCAGATGGTCGTACAGCGGACGGCCGTTCCGCGGATGAAGTTGTGGCAGGTCTT | |
| | | GTCGATCACTTAGTCGATAGTATGACCCCGGCAGATGATCGTCTGTGGCCGGTA | |
| | | AGCACTCTTACGGGAGAATCGGATCCATGTACAGTCCAGCAAGGCGCTGCTGGG | |
| | | GTGCTTGCGGTGTTGACCCGCTACTTCGAATTGACGGGCGATCCGCGCTTACCA | |
| | | GGCTTATTGTCGACAGCCGGACGTTGGATCGCAGACCGCACGGATGTTCGTTCA | |
| | | CCTCGTCCGGGATTACATTTCGGGGGACGCGGAACAGCCTGGGCCTTATACGAC | |
| | | GCGGGGCGTGCAGTCGACGATCGTCGCTTGGTGGAACATGCTCTGGACTTAGCA | |
| | | TTAGCCCCGCCCCAAGCGACTCCTCATCACGATGTCACGCATGGGACTGCGGGC | |
| | | TCAGGCTTAGCCGCCTTGCACCTGTGGCAGCGTACTGGAGATACTCGTTTCGCG | |
| | | GATTTAGCAGTAGAGGCAGCTGATCGCTTAACAGCTGCAGCTCGTCGCGAGCCT | |
| | | TCGGGTGTTGGATGGGCAGTACCTGCAGAGGCCGACTCCCCAGAAGGAGGCAAG | |
| | | CGTTACCTGGGCTTCGCTCATGGCGCAGCTGGGATTGGGTGCTTCTTATTGGCT | |
| | | GCGGCGGAACTTAGTCGTCAACCCGATCATCGTGCAACTGCTTTGGAAGTTGGC | |
| | | GAAGGCCTGGTTGCTGATGCTGTTCGCATCGGAGAGGCGGCACAGTGGCCTGCG | |
| | | CAATCCGGGGACTTGCCGACAGCGCCTTACTGGTGCCATGGGGCGGCAGGTATC | |
| | | GGGACATTTCTTGTACGCTTATGGCAGGCGACCGGGGACGATCGCTTCGGTGAT | |
| | | CTGGCCCGCGGGAGTGCTCACGCTGTGGCCGAACGTGCTAGTCGCGCCCCATTG | |
| | | GCGCAATGTCACGGTTTGGCTGGAAACGGAGATTTCTTGTTGGATTTGGCAGAC | |
| | | GCGACAGGCGATCCTGTGCATCGCGACACCGCGGAAGAGTTAGCAGGGTTGATC | |
| | | TTGGCCGAAGGAACCCGTCGTCAGGGACATGTCGTTTTCCCTAATGAGTATGGG | |
| | | GAAGTATCATCTTCATGGTCCGACGGTAGTGCGGGGATTCTTGCGTTCCTTCTG | |
| | | CGTACGCGTCATACGGGCCCTCGCCATTGGATGGTAGAACAACGTGGGTAATAA | |
| pEG7144 | RaxST | 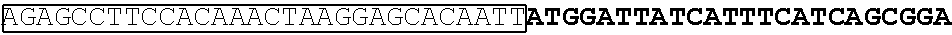 | 704 |
| | | CTGCCTCGTGCGGGGAGTTCATTACTGGCTGCGTTACTGCGTCAAAATCCGCAG | |
| | | CTGCATGCCGATGTTACATCTCCGGTGGCGCGCCTTTACGCGGCCATGCTGATG | |
| | | GGTATGAGTGAAGAACACCCGAGCAACGTGCAGATTGACGATGCCCAACGTGTC | |
| | | CGTCTGTTACGTGCAGTATTTGATGCGTATTATCAGAACCGTCAGGAACTGGGG | |
| | | ACAGTGTTCGATACTAACCGCGCATGGTGCTCTCGCCTCACGGGCCTGGCGCGT | |
| | | CTGTTTCCGCGTAGTCGCATGATCTGCTGTGTACGCGATGTGGGCTGGATTGTT | |
| | | GATTCTTTTGAACGCCTGGCGCAGTCGCAGCCGTTACGCCTTTCGGCCCTGTTC | |
| | | GGTTACGACCCCGAGGATTCGGTTAGCATGCACGCTGACTTACTCACTGCGCCT | |
| | | CGCGGGGTAGTGGGCTACGCCCTGGATGGTTTACGTCAAGCGTTTTATGGAGAT | |
| | | CACGCGGATCGTCTGCTGTTGTTACGTTATGATACGCTGGCACAGCGTCCTGCA | |
| | | CAAGCCATGGAACAGGTATATGCATTCCTGCAGCTCCCTGCCTTTGCACATGAT | |
| | | TATGCCGGTGTTCAGGCCGAAGCGGAACGCTTTGATGCCGCCCTGCAAATGCCT | |
| | | GGTTTGCACCGCGTGCGTCGTGGTGTTCACTATGTTCCGCGACGTTCGGTTTTA | |
| | | CCGCCTGCCCTGTTTGACCAGCTGCAGGAACTTGCATTCTGGGAAAGTGCACCC | |
| | | AGCCATGGAGCGCTGCTCGTGTAATAA | |
| pEG7145 | ComQ | 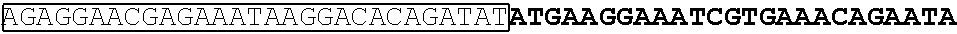 | 705 |
| | | TCAGTAACAAAGACCTGTCGCAACTCCTGTGTTCCTTCATTGATTCAAAGGAAA | |
| | | CTTTCAGTTTTGCCGAGAGCGCTATACTGCATTATGTAGTATTCGGCGGTGAGA | |
| | | ACCTGGACGTAGCTACCTGGCTGGGCGCCGGAATTGAAATTCTGATCCTGAGCA | |
| | | GCGATATCATGGACGACCTGGAGGACGAGGATAACCATCATGCGTTGTGGATGA | |
| | | AAATTAACCGCAGCGAGAGCTTGAATGCGGCCCTGTCCTTATACACCGTCGGCT | |
| | | TAACGAGCATCTATTCCCTGAACACAAATCCGTTGATATTTAAGTATGTGCTGC | |
| | | GCTACGTCAATGAGGCCATGCAGGGTCAGCATGATGATATAACCAATAAAAGCA | |
| | | AAACCGAAGATGAATCGCTTGAAGTGATTCGCCTTAAATGCGGCAGCCTGATCG | |
| | | CCCTGGCAAATGTCGCGGGCGTGCTGTTAGCCACGGGCGAGTACAATGAAACAG | |
| | | TTGAACGTTACTCTTATTACAAAGGCATCGTGGCGCAAATTTCCGGCGACTATC | |
| | | ACGTGCTGCTGTCAGGAAACCGGAGCGATATCGAGAAAAACAAACAGACACTGA | |
| | | TTTACCTGTATCTGAAACGCCTGTTTAACAACGCGAGCGAGGAATTGCTGTATC | |
| | | TGTTCTCCCATAAAGATTTGTACTATAAAGCCCTGCTCGACCGTGAAAAGTTTG | |
| | | AAGAAAAACTGATCCAGGCCGGGGTGACGCAGTACATCAGCGTTCTGCTCGAAA | |
| | | TATATAAGCAGAAGTGCTTCTCCACCATAGAACAGCTGAACTTAGATAAAGAAA | |
| | | AGAAAGAGCTGATCAAGGAGAGCCTGCTGTCATATAAGAAAGGCGACACCCGTT | |
| | | GCAAGACCTAATAA | |
| pEG7146 | KgpF | 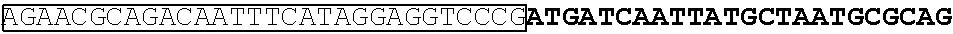 | 706 |
| | | CTCCATAAGAGTAAAAACTTGATGTATATGAAAGCCCACGAAAACATCTTCGAA | |
| | | ATCGAGGCGCTGTACCCGCTGGAATTGTTCGAGCGTTTTATGCAGTCCCAAACC | |
| | | GATTGCTCCATCGATTGTGCCTGTAAAATTGATGGTGACGAATTGTATCCCGCC | |
| | | CGTTTTAGTCTGGCCCTGTATAACAACCAGTATGCCGAAAAGCAAATTCGCGAA | |
| | | ACCATCGACTTCTTCCATCAGGTAGAGGGTCGGACCGAGGTGAAACTGAACTAT | |
| | | CAGCAACTGCAGCACTTCCTGGGTGCTGACTTCGATTTTAGCAAAGTGATTCGA | |
| | | AACCTGGTGGGTGTGGATGCACGCCGCGAACTGGCTGATTCCCGGGTTAAACTG | |
| | | TATATTTGGATGAACGATTACCCAGAGAAAATGGCGACCGCCATGGCATGGTGC | |
| | | GATGATAAGAAGGAATTGTCGACGTTGATAGTAAATCAGGAGTTTCTGGTCGGG | |
| | | TTCGATTTTTATTTCGATGGTCGCACGGCAATAGAATTATACATTAGTCTGTCA | |
| | | TCCGAAGAATTTCAGCAGACACAAGTTTGGGAACGCCTCGCAAAGGTAGTGTGC | |
| | | GCCCCAGCGCTGCGCCTTGTTAATGATTGCCAGGCGATCCAGATTGGCGTGAGC | |
| | | CGTGCCAATGATAGTAAGATCATGTATTACCATACCCTTAATCCGAACTCGTTT | |
| | | ATCGACAATCTGGGCAATGAAATGGCAAGCAGAGTTCACGCGTATTACCGACAT | |
| | | CAACCGGTTCGCTCTCTGGTAGTATGCATACCAGAACAGGAGTTGACCGCCCGG | |
| | | TCCATACAGCGCTTAAACATGTATTACTGTATGAACTAATAA | |
| pEG7147 | TgnB | 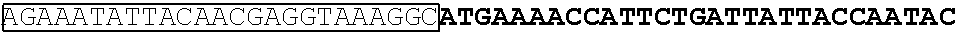 | 707 |
| | | CCTGGATCTGACCGTGGATTATATTATTAATCGCTATAATCATACCGCTAAATT | |
| | | TTTTCGTCTGAATACCGATCGTTTTTTTGATTATGATATTAATATTACCAATAG | |
| | | CGGTACCAGCATTCGTAATCGTAAATCTAATCTGATTATTAATATTCAGGAAAT | |
| | | TCATAGCCTGTATTATCGCAAAATTACCCTGCCGAATCTGGATGGCTATGAAAG | |
| | | TAAATATTGGACCCTGATGCAGCGCGAAATGATGAGTATTGTTGAAGGCATTGC | |
| | | AGAAACCGCTGGCAATTTTGCACTGACCCGTCCGTCTGTGCTGCGCAAAGCTGA | |
| | | TAATAAAATTGTGCAGATGAAACTGGCAGAAGAAATTGGTTTTATTCTGCCGCA | |
| | | GAGTCTGATTACCAATTCAAATCAGGCGGCAGCCTCATTTTGCAATAAAAATAA | |
| | | TACCAGCATTGTGAAACCGCTGAGTACCGGCCGCATTCTGGGTAAAAATAAAAT | |
| | | TGGCATTATTCAGACCAATCTGGTTGAAACCCATGAAAATATTCAGGGCCTGGA | |
| | | ACTGTCTCCGGCTTATTTTCAGGATTATATTCCGAAAGATACCGAAATTCGTCT | |
| | | GACCATTGTTGGTAATAAACTGTTTGGCGCCAATATTAAATCAACCAATCAGGT | |
| | | TGATTGGCGCAAAAATGATGCACTGCTGGAATATAAACCGGCCAATATTCCGGA | |
| | | TAAAATTGCCAAAATGTGTCTGGAAATGATGGAAAAACTGGAAATTAATTTTGC | |
| | | GGCGTTTGATTTTATTATTCGTAATGGTGATTATATTTTTCTGGAACTGAATGC | |
| | | CAATGGTCAGTGGCTGTGGCTGGAAGATATTCTGAAATTTGATATTTCAAATAC | |
| | | CATTATTAATTATCTGCTGGGTGAACCGATTTAATAATAA | |
| pEG7149 | PapB | 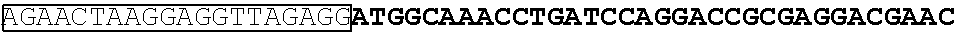 | 708 |
| | | TGATTCATTTCCATCCGTACAAACTGTTCGAGGTGGATTCAAAAACCTTCTTCT | |
| | | ATAACGTAGTCACCAACGCGATTTTTGAAATTGATAGCCTGATAATCGACATTC | |
| | | TTCACTCAAAAGGTAAAAATGAGGAGCACGTTGTGAAAGATTTGGCTGAACGCT | |
| | | ATGAGCTGTCTCAGGTTCGCGAAGCGATCCAGAACATGAAAGAGGCATACATTA | |
| | | TAGCAACCGATGCTAACATCTCCGACGTAGAGAAGATGGGTATCTTAGATAACT | |
| | | CGCAGCGCGTTTTTAAACTGTCTAGCCTGACGCTCTTTATGGTGCAGGAATGCA | |
| | | ACCTGCGGTGTACGTATTGTTACGGCGAAGAAGGAGAATACAACCAGAAAGGTA | |
| | | AAATGACGTCCGAAATCGCCCGGAGCGCAGTGGATTTTCTGATTCAACAGAGTG | |
| | | GTGAAATCGAACAGTTGAACATCACATTCTTTGGAGGCGAACCGCTGCTCAACT | |
| | | TTCCATTAATACAAGAAACCGTGCAGTATGTGCACGAACAGAGCGAGATCCATA | |
| | | ACAAGAAATTTAGCTTTTCCATCACCACCAATGGCACGCTCATTACCCCCAAAA | |
| | | TCAAAAACTTCTTCTATAAACACCACTTTGCAGTCCAGACTTCTATCGATGGTG | |
| | | ATGAAAAGACGCACAATTTCAATCGCTTCTTCAAAGGAGGCCAGGGCTCTTATG | |
| | | ATCTGCTGTTAAAGCGGACGGAAGAAATGCGCAATGACCGTAAAATTGGTGCAC | |
| | | GTGGAACCGTGACCCCTGCCGAGCTGGACCTCTCAAAATCATTTGACCACTTAG | |
| | | TTAAACTCGGCTTTCGCAAAATCTACTTATCACCCGCTTTATATAGTCTCTCTG | |
| | | ACGATCACTACGACACCCTGAGCAAAGAGATGGTCAAACTTGTTGAACAATTCC | |
| | | GTGAGCTGCTGGAGCGTGAAGATTACGTCACCGCGAAGAAAATGTCTAATGTTC | |
| | | TGGGTATGTTATCGAAGATTCACTCCGGTGGCCCGCGCATTCATTTTTGCGGTG | |
| | | CCGGCACTAATGCTGCCGCTGTCGATGTCCGCGGCAACCTTTTCCCGTGTCATC | |
| | | GTTTCGTGGGTGAAGATGAATGTTCAATCGGTAACCTGTTCGACGAGGACCCGC | |
| | | TGTCAAAACAGTACAACTTTATAGAGAATTCTACAGTACGCAACCGTACTACGT | |
| | | GTTCGAAATGCTGGGCGAAGAATCTGTGCGGCGGTGGTTGTCACCAAGAAAATT | |
| | | TCGCCGAGAATGGTAATGTGAACCAGCCAGTGGGCAAATTATGCAAAGTGACCA | |
| | | AAAACTTCATCAACGCGACCATCAATCTGTACTTGCAACTTACTCAAGAACAAC | |
| | | GCAGCATTCTGTTCGGCTAATAA | |
| pEG7152 | PcpXY | 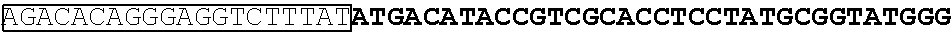 | 709 |
| | | AGATCACGCTGAAATGCAATCTGGCATGTTCGCACTGTGGAAGTCGTGCCGGGC | |
| | | ACACGCGAGCAAAAGAACTGTCCACACAGGAAGCGCTGGATCTGGTCCGTCAGA | |
| | | TGGCTGATGTCGGCATTATCGAAGTTACTCTGATTGGGGGTGAAGCGTTCCTGC | |
| | | GTCCAGACTGGCTGCAGATTGCCGAGGCGATAACGAAAGCCGGGATGCTGTGCA | |
| | | GCATGACTACGGGCGGTTATGGCATATCGCTGGAAACCGCCCGCAAAATGAAAG | |
| | | CGGCAGGAATCGCGAGCGTGAGCGTTAGCATCGATGGCTTGGAGGAAACCCATG | |
| | | ATCGCTTACGCGGTCGCAAAGGCTCTTGGCAGGCTGCGTTTAAAACAATGAGCC | |
| | | ATTTGAGAGAAGTGGGCATCTTCTTTGGCTGTAACACCCAGATTAACCGTCTGT | |
| | | CGGCCCCTGAATTTCCGCTGATATATGAACGCATCCGTGACGCCGGGGCACGTG | |
| | | CCTGGCAGATCCAGCTTACGGTGCCGATGGGCCGCGCTGCCGATAACGCAAATA | |
| | | TCCTTCTGCAACCGTACGAACTGCTTGATCTGTATCCGATGATTGCTCGAGTGG | |
| | | CCCGCCGGGCCCGTCAAGAGGGCGTGCAAATCCAGCCAGGTAATAATATTGGGT | |
| | | ATTACGGCCCTTACGAACGTCTTTTACGTGGCCGGGGGAGCGATAGTGAGTGGG | |
| | | cattttggcagggctgtgccgcgggcttaagtaccctgggtattgaagcggatg | |
| | | GTGCTATAAAAGGTTGTCCCTCACTGCCAACGAGCGCGTATACCGGCGGTAACA | |
| | | TTCGCGAACATAGTCTGCGAGAAATAGTGGAAGAATCGGAACAGCTGCGTTTTA | |
| | | ACCTCGGTGCAGGGACGAGCCAAGGGACCGCCCACTTGTGGGGCTTTTGCCAGA | |
| | | CGTGTGAATTTAGTGAATTGTGCAGAGGTGGTTGTACGTGGACAGCTCACGTGT | |
| | | TCTTTAACCGCCGTGGGAATAACCCGTATTGTCATCATCGGGCGCTTTTCCAAG | |
| | | CGGAGCAGGGTATCAGAGAACGTGTCGTGCCAAAGGTCGAAGCTCAGGGCCTGC | |
| | | CGTTTGACAACGGTGAATTTGAACTTATCGAAGAACCTATTGACGCGCCTCTGC | |
| | | CCGAAAATGATCCACTGCACTTTACCAGCGACTTAGTGCAGTGGTCAGCGAGTT | |
| | | 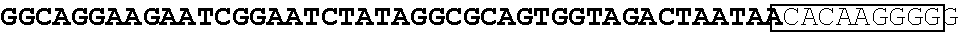 | |
| | | 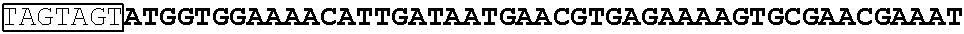 | |
| | | TGAACCGGAAAGCCTGCTTCTGCCGCGCCAGGCTTGGCAGTCGCAGATCGCCTA | |
| | | TCTTAAAGCGATTCTGAAAGCCAAACAGGCGCTTGACCGGATCGAAAAACGTTA | |
| | | TCTGCGGTAATAA | |
| pEG7160 | LynD | 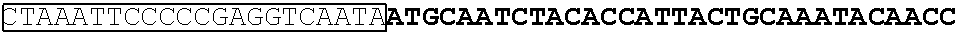 | 710 |
| | | ACATTTCCATGTAGAGGTCATTGAACCAAAGCAAGTCTACTTGTTGGGTGAACA | |
| | | AGCTAATCATGCATTGACAGGCCAATTATACTGCCAAATTTTGCCATTGTTAAA | |
| | | CGGACAATACACATTGGAACAAATCGTTGAAAAACTAGACGGAGAAGTACCACC | |
| | | TGAATACATTGATTATGTGCTGGAGAGACTAGCTGAGAAGGGCTATCTGACTGA | |
| | | AGCAGCACCTGAATTATCTAGTGAAGTGGCCGCTTTCTGGTCTGAGCTGGGGAT | |
| | | TGCACCTCCTGTCGCGGCCGAAGCATTACGTCAACCTGTGACTTTAACACCTGT | |
| | | TGGAAACATCAGCGAAGTAACAGTAGCAGCCTTAACCACAGCCCTACGTGATAT | |
| | | CGGTATTTCCGTTCAAACACCTACAGAAGCTGGATCGCCAACTGCATTGAACGT | |
| | | TGTACTTACCGATGATTATCTCCAACCAGAACTCGCTAAGATCAATAAGCAAGC | |
| | | CTTAGAAAGTCAACAAACTTGGCTACTTGTCAAACCAGTTGGCTCCGTGTTATG | |
| | | GTTGGGTCCGGTATTCGTGCCAGGAAAAACAGGTTGCTGGGATTGTTTGGCTCA | |
| | | CAGATTAAGGGGGAATAGAGAGGTAGAGGCCTCTGTATTGAGACAAAAACAAGC | |
| | | TCAACAACAACGTAATGGACAAAGCGGGTCTGTAATAGGATGCCTTCCCACGGC | |
| | | TAGAGCGACACTGCCCTCAACACTCCAAACTGGGCTGCAGTTCGCTGCTACCGA | |
| | | AATTGCTAAATGGATAGTTAAGTATCATGTTAATGCCACAGCGCCTGGCACCGT | |
| | | ATTCTTCCCTACATTGGATGGTAAGATAATTACGCTAAATCACTCCATACTGGA | |
| | | TTTGAAGTCACATATTCTGATCAAGCGTTCTCAATGTCCCACCTGTGGTGACCC | |
| | | AAAAATCTTACAGCACCGTGGTTTCGAACCTTTAAAACTTGAGTCAAGGCCTAA | |
| | | ACAGTTCACCTCAGACGGCGGACATCGTGGTACTACCCCTGAACAAACTGTCCA | |
| | | GAAATATCAACATTTAATCTCGCCTGTTACCGGTGTAGTTACTGAATTGGTCAG | |
| | | GATAACTGATCCGGCCAATCCACTAGTTCACACATATAGAGCTGGTCATAGCTT | |
| | | CGGGAGCGCTACATCGCTGAGAGGGCTGCGTAATACCTTAAAGCATAAGAGTTC | |
| | | AGGTAAGGGTAAGACTGATTCTCAAAGTAAAGCCTCGGGCCTGTGTGAGGCGGT | |
| | | AGAACGTTACTCAGGAATCTTTCAAGGTGACGAACCGAGAAAACGCGCCACATT | |
| | | GGCTGAATTGGGAGATTTGGCAATTCACCCTGAGCAATGCTTGTGTTTTTCCGA | |
| | | CGGTCAGTACGCTAATAGAGAAACTTTAAACGAACAGGCAACGGTGGCACATGA | |
| | | TTGGATACCTCAACGTTTTGATGCATCACAAGCTATTGAATGGACTCCAGTCTG | |
| | | GTCCCTAACTGAACAGACCCATAAATATTTGCCCACCGCATTGTGTTACTACCA | |
| | | TTATCCTCTACCCCCAGAACACAGATTCGCACGTGGAGATTCGAATGGTAATGC | |
| | | TGCCGGAAATACGTTGGAAGAGGCTATACTCCAAGGCTTCATGGAATTAGTCGA | |
| | | GAGAGATGGTGTGGCTTTATGGTGGTATAACAGGCTACGCAGACCCGCTGTAGA | |
| | | CTTAGGCTCATTTAACGAGCCATACTTCGTTCAGTTGCAACAATTCTACAGAGA | |
| | | AAACGATAGAGATTTGTGGGTTTTGGACTTGACAGCTGATTTAGGTATCCCGGC | |
| | | TTTCGCGGGCGTTTCTAATAGAAAAACTGGTAGTTCGGAGAGGTTGATATTAGG | |
| | | ATTCGGTGCACACCTCGATCCTACTATTGCAATTCTGAGAGCAGTTACAGAAGT | |
| | | TAACCAGATTGGCCTTGAATTAGATAAAGTTCCAGACGAGAACCTTAAGAGCGA | |
| | | CGCAACAGATTGGCTAATTACTGAAAAATTAGCTGACCACCCTTATTTGTTACC | |
| | | AGATACAACTCAACCTCTAAAAACTGCTCAAGATTATCCTAAAAGGTGGTCTGA | |
| | | CGATATATACACGGACGTAATGACTTGCGTTAATATTGCTCAACAAGCAGGACT | |
| | | TGAAACTCTAGTTATTGATCAAACACGTCCGGACATTGGTTTGAATGTTGTTAA | |
| | | GGTGACAGTCCCGGGGATGAGGCACTTTTGGTCAAGATTTGGAGAGGGGAGGCT | |
| | | TTATGACGTGCCCGTCAAATTAGGTTGGCTTGACGAACCTTTGACCGAAGCGCA | |
| | | AATGAACCCCACGCCGATGCCTTTTTAATAA | |
| pEG7166 | PapoK | 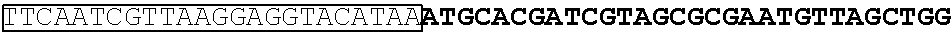 | 711 |
| | | ACCAAATACATCGCGTTTGGTCTGCGCATTGCCAGCGAACTCAACTTACCGGAA | |
| | | CTGATATTGGCGGCTCCCGAAGCCGTTGAGGATGTTGTCATACGCCAGGCAGAT | |
| | | CTCACGGCCTGGTCTGGCCAACTTGAACAGGCAAATTTTGTCATGTTGGACGAA | |
| | | CGTTTCATGTTTCAGATCCCGGGGACCGCCATTTATGCGGTACGCGAAGGCAAA | |
| | | GAGATTGAAGTGAGCATCTTCTCTGGGGCCGACCCGGACACCGTGCGCCTTTTC | |
| | | GTGCTGGGGACGTGCATGGGCGTGCTCTTGATGCAGCGCCGCATTCTGCCTATC | |
| | | CACGGCTCCGCCGTCGTTATCGGTGGCCGCGCGTATGCCTTTGTTGGTGAATCA | |
| | | GGCACAGGTAAATCGACCTTAGCTGCAGCATTTCGGCAGGCCGGTTACCAAATG | |
| | | GTTAGCGATGATGTCATTGCCGTCAAAGCGACCGCATCTAGCGCTATTGTTTAC | |
| | | CCTGCGTATCCACAGCAAAAACTGGGTTTAGATTCGCTGTTGCAGCTTGAAGCG | |
| | | CTCCGTGAGAATAAGCACGCCCGCAAGCGTAACAACATCCGTTCTCTGACGGAT | |
| | | GGCAATAGTGTGATGCCGCAGTACAGCGATCTGCGCATGCTGGCGGGGGAACTG | |
| | | AATAAATATGCAGTTCCAGCCGTCGATGAATTCTTTAATGACCCGCTGCCGTTG | |
| | | GGCGGTGTTTTCGAACTGGTAGCAGACAGTCCGATTCGAGCATTAATGCGCGAA | |
| | | GGCGAACTCGTCGCTGTGACCGAGCAACCGCTGAACGTTCTGGAATGTTTACAT | |
| | | ACTCTTCTGCAACACACGTACCGTCGGGTAATCATCCCTCGAATGGGACTGAGC | |
| | | GAGTGGAGCTTCGATACTGCGGCCCGAATGGCACGCAAGGTCGAGGGCTGGCGA | |
| | | CTCCTCCGTGATAGCTCCGTGTTCACGGCTAGTGAAGTCGTCCAGCGCGTCCTC | |
| | | GACATCATCCGTAAGGAGGAAAAGAGCTACGGATCACACTAATAA | |
| pEG7169 | EpiD | 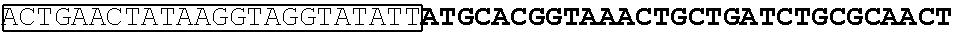 | 712 |
| | | GCTTCGATCAACGTCATCAATATCAACCATTATATTGTGGAGCTGAAACAGCAC | |
| | | TTCGATGAGGTGAATATCCTGTTTTCACCTTCCTCGAAGAACTTTATCAACACC | |
| | | GATGTCCTGAAGCTGTTTTGCGATAATCTGTATGACGAGATCAAAGATCCGCTG | |
| | | CTGAACCACATCAACATAGTGGAGAACCACGAGTATATCTTGGTGCTGCCTGCC | |
| | | AGTGCCAATACGATCAACAAAATCGCGAACGGTATATGCGATAACCTCTTGACG | |
| | | ACCGTATGCTTAACCGGGTACCAGAAACTGTTTATCTTTCCGAATATGAACATC | |
| | | CGCATGTGGGGAAATCCGTTCTTACAGAAAAATATTGACCTGCTTAAAAGCAAC | |
| | | GACGTGAAGGTGTATTCCCCCGACATGAACAAATCTTTTGAGATAAGCTCAGGC | |
| | | CGCTACAAAAATAACATCACGATGCCGAATATCGAAAACGTGCTGAATTTTGTC | |
| | | CTGAACAATGAGAAACGCCCGCTGGATTAATAA | |
| pEG7171 | BamB | 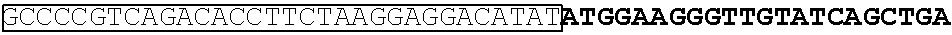 | 713 |
| | | AAGTGCATAGTCGTATACACAAACTGCAAAATAATATCGCAATAGGTAGCATGC | |
| | | CGCCTCACGCGCTGATCATCGAGGATGCCCCCGAATATTTGTCAAACGTTCTGC | |
| | | GCTTCTTTAGTAGCAAAAAGACTATAAAAGAAGCTGAAGTGTACCTGTCGGATA | |
| | | ATACGAATCTGAGCTCCAATGAGATCAACCTGTTGTTAGGTGATCTGATTGAGA | |
| | | ACGAGATTATCGTAAAGCAAAACTACGACTCGAATAATCGGTACAGTCGACACA | |
| | | GTCTGTATTACGAGATGATTGATGCCAACGCTGAAAACGCGCAGAAAATTCTGG | |
| | | CAGAGAAAACAGTGGGCCTCGTTGGGATGGGCGGGATTGGTTCCAATGTAGCCA | |
| | | TGAATCTCGCAGCCGCCGGTGTTGGCAAACTGATCTTTAGTGATGGCGATACCA | |
| | | TAGAACTGTCTAATTTAACGCGACAGTATCTTTACAAAGAGGATCAGGTGGGCT | |
| | | TGAGCAAAGTAGAGAGCGCCAAAGAACAACTGCAATTACTGAATAGCGAAGTCG | |
| | | AGCTTATCCCGGTTTGCGAAAGTATCTCTGGTGAGGAACTGTTCGACAACCATT | |
| | | TCTCCGAATGCGATTTCGTCGTACTGTCCGCCGACTCTCCGTTCTTTGTTCACG | |
| | | AATGGATTAACAATGCCGCGTTGAAATATGGCTTCTCCTACTCTAACGCAGGAT | |
| | | ATATCGAAACCTATGGCGCGATCGGTCCACTGGTGATACCTGGGGAAACTGCCT | |
| | | GCTACGAATGCTATAAAGACAAGGGCGATCTTTACTTGTACTCCGACAACAAGG | |
| | | AAGAATTTTCTGTGAACCTGAATGAATCATTCCAAGCACCGAGCTATGGACCGC | |
| | | TTAATGCGATGGTTAGTTCCATTCAGGCGAATGAAGTGATACGCCACCTCCTCG | |
| | | GACTTAAAACCAAAACGTCCGGCAAACGGCTGCTGATCAACAGTGAAATCTACA | |
| | | AAATCCACGAAGAGAACTTCGAGAAGAAGAACAACTGCCTGTGCTCGGATATTA | |
| | | AGGGCGAGAAGCTGTCGAAGAACACCCTTAACTCCGATAAAGAGCTGCACGAAG | |
| | | TGTATATCGAAGAACGCGAATCGGATTCTTTCAACTCCATTCTCTTGGATAAAA | |
| | | CCATGAGCAAGCTGGTAAAAATTAACAAAGAGGAGACAAAAATCCTCGACATTG | |
| | | GTTGCGCTACCGGCGAACAGGCTCTGTATTTCGCGAATAAAGGTGCTAAGGTGA | |
| | | CCGCTGTCGACATTTCAGACGATATGTTGAAGGTGCTGGACAAGAAAGCAAGCA | |
| | | ACATTAACGCGGGGAGTATCAAAACCATGCGTGGTAATATCGAATCCATCGAGG | |
| | | TGAATGACACTTTTAATTACATCGTCTGTAACAACATCCTTGATTACCTGCCGG | |
| | | AGATCGACCGCACGCTGAGAAAACTTAACATGTTTTTGAAAAATGACGGGACGC | |
| | | TGATTGTGACGATTCCCCACCCCGTGAAGGATGGTGGAGGGTGGCGGAAAGATT | |
| | | ATTATAACGGCAAATGGAACTACGAAGAGTTTATCCTGAAGGATTACTTCAACG | |
| | | AGGGTCTGATCGAAAAGAGCCGCGAGGACAAAAATGGGGAAACGGTGATCAAAA | |
| | | GCATTAAAACGTACCACAGAACCACCGAAACCTATTTCAATAGCTTTACTGACG | |
| | | CTGGCTTCAAGGTAGTATCTCTGCTGGAACCGCAACCGCTTTCAACTGTTTCAG | |
| | | AGACTCATCCAATTCTGTTCGAAAAGTGTTCGCGCATTCCGTACTTTCAAGTTT | |
| | | TTGTGCTCAAGAAAGAGGATCGCCACGCCATTTAATAA | |

^{a}In each backbone sequence (labeled “bEG_SX”, where X is a number), the relevant part (encoding a peptide or RBS + enzyme) has GFP as a placeholder (RBS + GFP for enzyme plasmid) and is double underlined. This region can be replaced with an insert DNA (such as a peptide or RBS + enzyme, including those listed below each plasmid backbone sequence) to get a plasmid sequence. The full plasmid sequences used herein (labeled “pEG####”) for peptides and enzymes can be identified by replacing the double underlined portion of the backbone sequence found above a given peptide/RBS + enzyme sequence with the respective peptide/RBS + enzyme sequence (for example, the full pEG3045 plasmid sequence is provided by replacing the double underlined portion of the bEG_S2 backbone sequence with the HIS₆-MdnA provided next to the pEG3045 label).
^{b}Text is formatted according to sequence components: promoters (lowercase), ribozyme 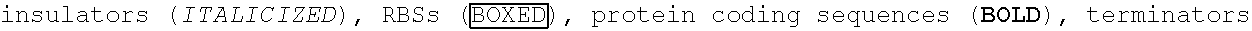
(UNDERLINED), and plasmid backbone and spacers (REGULAR ALL CAPS).

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

What is claimed is:

1. A non-naturally occurring peptide comprising:
(A) AACX₁X₂X₃X₄X₅X₆MPPX₇X₈X₉X₁₀X₁₁X₁₂C (SEQ ID NO: 1) (scaffold L1), wherein:
(i) X₆ and X₇ are each the amino acid S or T;
(ii) X₁-X₅ and X₈-X₁₂ are each any amino acid; and
(iii) the peptide comprises a thioether bridge that links C at position 3 in to S or T at position 9 in SEQ ID NO: 1 and a thioether bridge that links S or T at position 13 to C at position 19 in SEQ ID NO: 1;
(B) X₁PX₂TTX₃X₄TX₅X₆X₇EX₈X₉DX₁₀DEX₁₁X₁₂X₁₃ (SEQ ID NO: 2) (scaffold L2), wherein:
(i) X₂ is the amino acid H, Q, N, K, D, or E;
(ii) X₆ is the amino acid F, L, S, I, M, T, V, or A;
(iii) X₇ is the amino acid F, L, I, or V;
(iv) X₁, X₃-X₅ and X₈-X₁₃ are each any amino acid; and
(v) the peptide comprises an ester bridge that links T at position 5 of SEQ ID NO: 2 to D at position 15 of SEQ ID NO: 2 and an ester bridge that links T at position 8 of SEQ ID NO: 2 to E at position 12 of SEQ ID NO: 2;
(C) X₁CX₂X₃X₄X₅X₆CX₇X₈X₉X₁₀X₁₁ (SEQ ID NO: 3) (scaffold L3), wherein:
(i) X₅ and X₁₀ are each the amino acid D or E;
(ii) X₁-X₄, X₆-X₉, and X₁₁ are each any amino acid; and
(iii) the peptide comprises a thioether bridge that links C at position 2 to D or E at position 6 of SEQ ID NO: 3 and a thioether bridge that links C at position 8 to D or E at position 12 of SEQ ID NO: 3;
(D) X₁CX₂X₃CX₄X₅X₆X₇X₈X₉ (SEQ ID NO: 4) (scaffold L4), wherein:
(i) X₄ and X₇ are each the amino acid D or E;
(ii) X₁-X₃, X₅-X₆, and X₈-X₉ are each any amino acid; and
(iii) the peptide comprises a thioether bridge that links C at position 2 to D or E at position 6 of SEQ ID NO: 4 and a thioether bridge that links C at position 5 to D or E at position 9 of SEQ ID NO: 4; and/or
(E) X₁CX₂X₃X₄X₅X₆CX₇X₈CX₉X₁₀X₁₁X₁₂X₁₃ (SEQ ID NO: 5), wherein:
(i) X₅, X₉, and X₁₂ are each the amino acid D or E;
(ii) X₁-X₄, X₆-X₈, X₁₀-X₁₁, and X₁₃ are each any amino acid; and
(iii) the peptide comprises a thioether bridge that links the C at position 2 to D or E at position 6 of SEQ ID NO: 5, a thioether bridge that links C at position 8 of SEQ ID NO: 5 with D or E at position 12 of SEQ ID NO: 5, and a thioether bridge that links C at position 11 with D or E at position 15 of SEQ ID NO: 5.

2. The non-naturally occurring peptide of claim 1, comprising scaffold L5 and a sequence selected from SEQ ID NOS: 6-16; and/or scaffold L3 and a sequence selected from SEQ ID NOs: 17-25.

3. The non-naturally occurring peptide of claim 1 or 2, wherein the non-naturally occurring peptide comprises scaffold L3 and SEQ ID NO: 24.

4. A host cell comprising a heterologous nucleic acid encoding the non-naturally occurring peptide of any one of claims 1-3.

5. The host cell of claim 4, wherein the heterologous nucleic acid further encodes SEQ ID NO: 46.

6. The host cell of claim 4 or 5, wherein the heterologous nucleic acid comprises any one of SEQ ID NOs: 47-66.

7. A host cell comprising:
(a) a first fusion protein comprising (i) a first fragment of a transcription factor, (ii) a first split intein, and (iii) a target protein;
(b) a second fusion protein comprising (i) a candidate peptide, (ii) a second split intein, and (iii) a second fragment of the transcription factor; wherein the first split intein and second split intein are complementary fragments; and
(c) an inducible promoter operably linked to at least one reporter gene, wherein the transcription factor induces transcription of the at least one reporter gene when the transcription factor is present as a full-length transcription factor.

8. The host cell of claim 7, wherein:
(A) in (a), the first fusion protein comprises (i)-(iii) linked sequentially from the N-terminus to the C-terminus, the first fragment is an N-terminal fragment of the transcription factor and the first split intein is an N-terminal split intein; and
(B) in (b), (i)-(iii) are linked sequentially from the N-terminus to the C-terminus, wherein the second split intein is a C-terminal split intein, and the second fragment is a C-terminal fragment of the transcription factor; or
(C) in (a), from the N-terminus to the C-terminus, the first fusion protein comprises (iii) linked to (ii) linked to (i), wherein the first fragment is a C-terminal fragment of the transcription factor and the first split intein is a C-terminal split intein; and
(D) in (b), from the N-terminus to the C-terminus, the second fusion protein comprises (iii) linked to (ii) linked to (i), wherein the second split intein is an N-terminal split intein and the second fragment is an N-terminal fragment of the transcription factor.

9. The host cell of claim 7 or 8, wherein the cell is a eukaryotic or prokaryotic cell, optionally wherein the prokaryotic cell is a bacterial cell.

10. The host cell of any one of claims 7-9, wherein the transcription factor is a sigma factor (a factor).

11. The host cell of any one of claims 7-10, wherein the first fusion protein is encoded by a first heterologous nucleic acid and the second fusion is encoded by a second heterologous nucleic acid.

12. The host cell of any one of claims 7-11, wherein the candidate peptide comprises a sequence selected from SEQ ID NOs: 6-25 or comprises the non-naturally occurring peptide of any one of claims 1 or 2, optionally wherein the candidate peptide further comprises SEQ ID NO: 46.

13. The host cell of any one of claims 7-12, wherein the at least one reporter gene encodes a positive selection marker, a negative selection marker, and/or a fluorescent protein, optionally wherein the positive selection marker is an antibiotic resistance gene, optionally wherein the antibiotic resistance gene is chloramphenicol acetyltransferase (cat), optionally wherein the negative selection marker is the herpes simplex virus-thymidine kinase (hsvtk) gene.

14. The host cell of any one of claims 7-13, wherein the inducible promoter is an ECF promoter.

15. The host cell of any one of claims 7-14, wherein the target protein comprises a viral receptor binding domain (RBD) of the SARS-CoV-2 spike protein.

16. The host cell of claim 15, wherein the RBD comprises SEQ ID NO: 71.

17. The host cell of any one of claims 4-16, further comprising one or more enzymes selected from ProcM, LynD, TgnB, or PapB, optionally wherein the host cell comprises a heterologous nucleic acid encoding the enzyme, optionally wherein the heterologous nucleic acid encoding the enzyme comprises an inducible promoter.

18. A method of identifying a peptide that binds a target protein comprising culturing the host cell of any one of claims 7-17 and detecting transcription of the at least one reporter gene, thereby identifying the candidate peptide as being capable of binding to the target protein.

19. A method of identifying a peptide that binds a target protein comprising incubating in a reaction vessel:
(a) a first fusion protein comprising (i) a first fragment of a transcription factor, (ii) a first split intein, and (iii) a target protein;
(b) a second fusion protein comprising (i) a candidate peptide, (ii) a second split intein, and (iii) a second fragment of the transcription factor; wherein the first and second split inteins belong to the same intein; and
(c) an inducible promoter operably linked to at least one reporter gene, wherein the transcription factor induces transcription of the at least one reporter gene when the transcription factor is present as a full-length transcription factor, and detecting transcription of the reporter gene, thereby identifying the candidate peptide as being capable of binding to the target protein.

20. A method of treating a subject having or suspected of having a SARS-CoV-2 infection comprising administering an effective amount of the non-naturally occurring peptide of any one of claims 1-3.

21. The method of claim 18 or 19, wherein the method comprises: repeating the method with a plurality of candidate peptides.

22. The method of claim 18, or 21, wherein culturing comprises positive and/or negative selection of the host cell.

23. The method of claim 21 or 22, wherein the method further comprises sequencing.

24. A library comprising a plurality of peptides, wherein each peptide of the plurality of peptides has a length of n amino acids and has an amino acid sequence defined by a motif X₁X₂X₃X₄ . . . Xₙ, wherein n is 5-100, wherein each of X₁-Xₙ is independently selected from a group consisting of up to 20 amino acids and at least one of X₁-Xₙ within each peptide is an amino acid selected from a group consisting of 19 or fewer amino acids, and wherein the motif X₁X₂X₃X₄ . . . Xₙ is determined to be susceptible to post-translational modification by at least 2 distinct protein modification enzymes.

25. The library of claim 24, wherein less than 80% of the plurality of peptides are capable of being fully modified by the at least 2 distinct protein modification enzymes.

26. The library of claim 24 or 25, wherein at least one of X₁-Xₙ is defined to be a single amino acid.

27. A composition comprising a plurality of host cells, each host cell of the plurality comprising a peptide of the library of any one of claims 24-26, wherein the peptide comprised by each host cell is independent of the peptide comprised by each other host cell.

28. The composition of claim 27, wherein the composition comprises each peptide of the plurality of peptides.

29. The composition of claim 27 or claim 28, wherein the host cells are bacterial cells.

30. The composition of any one of claims 27-29, wherein the peptide is encoded by a first nucleic acid sequence in the host cell.

31. The composition of any one of claims 27-30, wherein each host cell further comprises at least one protein modifying enzyme.

32. The composition of claim 31, wherein the at least one protein modifying enzyme is encoded by a second nucleic acid sequence in the host cell.

32. A method of designing an amino acid motif, the method comprising:
(i) selecting one or more protein modifying enzymes;
(ii) identifying a recognition site (RS) sequence for each of the one or more protein modifying enzymes;
(iii) constructing a plurality of nucleic acid molecules, each nucleic acid molecule encoding a leader amino acid sequence comprising the RS sequence for each of the one or more protein modifying enzymes;
(iv) assigning a score to each of the plurality of nucleic acid molecules; and
(v) selecting one of the plurality of nucleic acid molecules based on step (iv),
to design the amino acid motif, wherein each RS sequence facilitates interaction of the corresponding protein modifying enzyme to a peptide defined by the amino acid motif, and wherein the leader amino acid sequence encoded by the nucleic acid molecule selected in step (v) is comprised within each peptide defined by the amino acid motif.

33. The method of claim 32, wherein each peptide defined by the amino acid motif further comprises a core sequence.

34. The method of claim 33, wherein the core sequence comprises one or more amino acids susceptible to modification by the one or more protein modifying enzymes.
